(19) United States
(12) Patent Application Publication
(10) Pub. No.: US 2026/0295049 A1
(43) Pub. Date: Oct. 1, 2026

(54) BENZOPORPHYRIN ANALOGS, CONJUGATES, AND METHODS OF USE THEREOF

(71) Applicant: Nira Biosciences, Inc., Burlington, MA (US)

(72) Inventors: Amissi Sadiki, Cambridge, MA (US); Zhaohui Sunny Zhou, Wellesley, MA (US); Shanshan Liu, Medford, MA (US)

(21) Appl. No.: 19/474,270

(22) PCT Filed: Apr. 12, 2024
(86) PCT No.: PCT/US2024/024423

§ 371 Date: Oct. 10, 2025

Related U.S. Application Data
(60) Provisional application No. 63/459,338, filed on Apr. 14, 2023.

(51) Int. Cl. A61K 41/00; A61K 47/68; C07D 487/22; C09K 11/06

(52) U.S. Cl. CPC A61K 41/0071; A61K 47/6849; A61K 47/6889; C07D 487/22; C09K 11/06; C09K 2211/1466

(57) ABSTRACT

The present disclosure provides photosensitizer-targeting agent conjugates (e.g., photosensitizer-antibody conjugates (PACs)) comprising a photosensitizer and a targeting agent (e.g., an antibody or antigen-binding fragment thereof) that binds a target molecule, e.g., an antigen expressed on cancer cells or immune cells. Also included in the present disclosure is photosensitizer compounds, photosensitizer-linker compounds, and modified-targeting agents that can be used for preparing the photosensitizer-targeting agent conjugates. Metabolites of the photosensitizer-targeting agent conjugates are also disclosed. The disclosure further relates to methods and compositions useful in the treatment and/or imaging of diseases or conditions that express a target molecule, as well as methods of making those compositions.

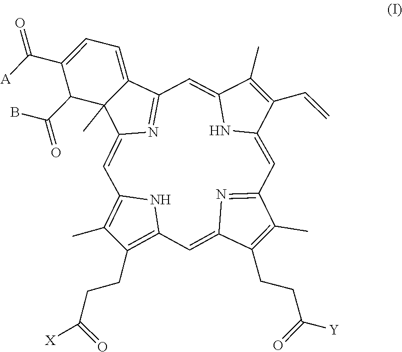

### RELATED APPLICATIONS

This application claims priority to U.S. Provisional Application No. 63/459,338, filed on Apr. 14, 2023. The entire contents of the foregoing application are expressly incorporated herein by reference.

### FIELD OF THE INVENTION

The present disclosure relates to photosensitizer-targeting agent conjugates (e.g., photosensitizer-antibody conjugates (PACs)) comprising a photosensitizer and a targeting agent (e.g., an antibody or antigen-binding fragment thereof) that binds a target molecule, e.g., an antigen expressed on cancer cells or immune cells. The disclosure further relates to methods and compositions useful in the treatment and/or imaging of diseases or conditions that express a target molecule, as well as methods of making those compositions.

### BACKGROUND OF THE INVENTION

Photodynamic therapy (PDT) is a therapeutic modality that combines a photosensitizer (PS) with non-ionizing light to kill cells and treat disease. The photosensitizer is administered and accumulates in the desired cells or tissues. Light-induced activation of the photosensitizer in the presence of oxygen generates reactive oxygen species (ROS) such as singlet oxygen (¹O₂) or free radicals that are toxic to the illuminated area but not to the rest of the body. One family of PS contains the core structure of benzoporphyrin (BP). For example, benzoporphyrin derivative (BPD, also verteporfin, marketed as Visudyne®), which is an FDA-approved photosensitizer to treat macular degeneration.

Non-targeted photosensitizers are poorly selective and often accumulates in healthy cells and results in unwanted off-target toxicity and photo-sensitivity. To solve this problem, photosensitizer-antibody conjugates (PACs, also referred to as photo-immunoconjugates or photo-immunotherapy) are molecularly targeted therapies that have been developed to impart cellular selectivity, improve pharmacokinetics, and reduce off-target toxicity. Furthermore, PACs provide two layers of selectivity to the treatment: (i) the targeting agent (such as an antibody) targets a desired cell and delivers the photosensitizer and (ii) light-induced activation of the photosensitizer kills the desired cell in a spatial and temporal manner.

A major impediment in the field is in the pharmaceutical development of PACs (e.g., synthesis, characterization, purification and storage). These shortcomings are underscored by the undesired physico-chemical properties of the photosensitizer BPD, which include: (i) BPD is hydrophobic and highly adsorptive, (ii) BPD tends to aggregate, and (iii) BPD forms non-covalent interactions such as pi-pi stacking. These physico-chemical properties make it difficult to conjugate BPD to antibodies or other proteins and/or to obtain well behaved conjugates. Previously, to overcome some of these shortcomings, large polymers were added to the PACs to improve the hydrophilicity of the PAC such as 10 kDa polyvinyl alcohol (PVA) or 10 kDa polyethylene glycol. Although these polymers succeeded in improving PAC solubility, the design and synthesis results in a heterogenous mixture, which ultimately renders the conjugates not developable for clinical use.

Accordingly, there is a need for new analogs of benzoporphyrin that has improved physico-chemical properties (e.g., hydrophilicity) for facile conjugation to the targeting agents, better properties of the conjugates, and better biological properties for clinical applications.

### SUMMARY OF THE INVENTION

To overcome challenges in the pharmaceutical development (e.g., synthesis, characterization, purification and storage) of PACs with BPD as the photosensitizer, the benzoporphyrin analogs of the present disclosure having improved physico-chemical properties are developed, which allows for facile conjugation to targeting agents (e.g., antibody or antigen-binding fragement thereof). Structural changes were made to BPD which include: (a) addition of short PEG units and/or introduction of the carboxylate and/or amide groups, and (b) the introduction of a bioconjugation handle to enable facile conjugation to targeting agents such as small molecules, peptides and proteins (see FIGS. 3, 4, 11, 17B, and 18B for structures of benzoporphyrin analogs and their corresponding conjugates). Non-specific conjugation of 2 antibodies—Panitumumab (Vectibix®) and anti-CD2 (clone: RPA-2.10)—have been successfully demonstrated. Non-specific methods involved acylation of lysine residues using N-hydroxysuccinimide (NHS) ester chemistry. The PACs exhibited cytotoxicity in an EGFR-positive and CD2-positive cancer cell lines (see FIGS. 7, 8, 10, 17, and 18). The data described herein demonstrated the applicability not only in cancer cells but also other aberrant cells, such as immune cells, for the PACs described herein. Furthermore, structure-activity relationship (SAR) of benzoporphyrin (BP) analogs conjugated to Panitumumab were evaluated in a cell-based assay and included: (a) a monoacid derivative (VertMA, i.e., least hydrophilic or most hydrophobic PAC design), (b) a diacid derivative (VertDA, i.e., moderately hydrophilic PAC design) and (c) a triacid derivative (VertTA, i.e., highly hydrophilic PAC design). Surprisingly, for both cleavable and non-cleavable linkers (see FIGS. 17 and 18), we observe that the most hydrophilic PAC design (VertTA) resulted in the least potent molecule, which also is the easiest BP analog to conjugate to an antibody. In other words, the least hydrophilic or most hydrophobic construct (VertMA) resulted in the highest potency observed. In the system presented herein, facile conjugation to an antibody and desired photoactivity can be achieved by selecting BP analogs with varying degree of hydrophilicity and hydrophobicity.

In one aspect, the present disclosure provides photosensitizer compounds that can be used for preparing photosensitizer-antibody conjugates (PACs) described herein. In some embodiments, the photosensitizer compound of the present disclosure is a benzoporphyrin analog.

In some embodiments, a photosensitizer compound is represented by Formula (I):

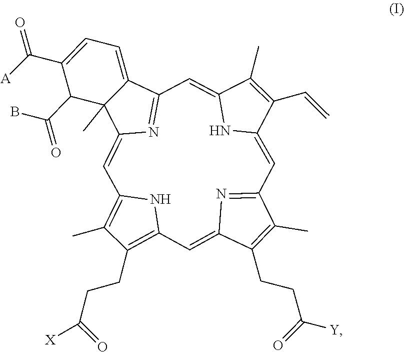

or a pharmaceutically acceptable salt thereof, wherein:
A, B, X and Y are each independently —OH, —OC₁₋₄alkyl, —N(R¹⁰⁰)(R¹⁰¹), or —Z-L^{P}-ker, provided at least one of A, B, X and Y is —N(R¹⁰⁰)(R¹⁰¹), —Z-L^{P}-NH₂, or —Z-L^{P}-ker, and provided at most one of A, B, X and Y is —Z-L^{P}-NH₂, —Z-L^{P}-ker;
R¹⁰⁰ is H or C₁₋₃alkyl;
R¹⁰¹ is H, C₁₋₃alkyl or —(CH₂—CH₂—O)ₙᵣR¹⁰²;
R¹⁰² is H or Me;
nr is an integer from 1 to 16; Z is —O— or —NR¹—;
R¹ is H or C₁₋₃alkyl;
L^{P} is a spacer;
ker is a reactive group.

In another aspect, the present disclosure provides a linker-photosensitizer compound of formula (II):

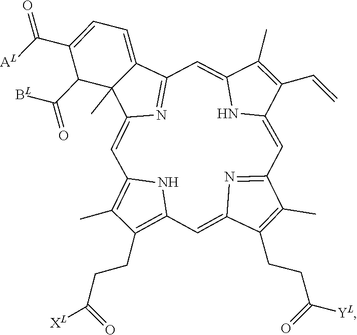

or a pharmaceutically acceptable salt thereof, wherein:
A^{L}, B^{L}, X^{L} and Y^{L} are each independently —OH, —OC₁₋₄alkyl, —N(R¹⁰⁰)(R¹⁰¹) or —Z-L^{P}-linker-L^{A}-R^{A}, provided one of A^{L}, B^{L}, X^{L} and Y^{L} is —Z-L^{P}-linker-L^{A}-R^{A};
R¹⁰⁰ is H or C₁₋₃alkyl;
R¹⁰¹ is H, C₁₋₃alkyl or —(CH₂—CH₂—O)ₙᵣR¹⁰²;
R¹⁰² is H or Me;
nr is an integer from 1 to 16;
Z is —O— or —NR¹—;
R¹ is H or C₁₋₃alkyl;
L^{P} and L^{A} are each independently a spacer;
Linker is connection moiety formed by a first reactive group attached to L^{P} and a second reactive group attached to L^{A}; and
R^{A} is a reactive group that can form a covalent bond with a targeting agent.

In yet another aspect, the present disclosure provides a conjuguate comprising a a benzoporphyrin analog covalently linked to a targeting agent, wherein the conjugate is represented by the following formula:

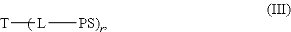

or a pharmaceutically acceptable salt thereof, wherein:
T is targeting agent;
L is a spacer connecting the targeting agent and PS;
r is an integer from 1 to 20;
PS is a photosensitizer that is a benzoporphyrin analog represented by the following formula:

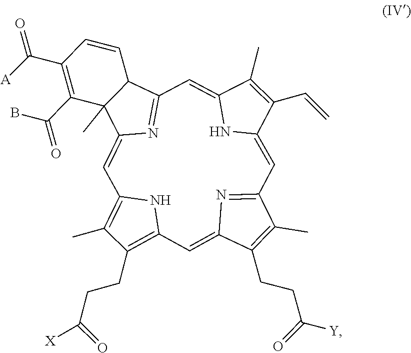

or a pharmaceutically acceptable salt thereof, wherein:
A, B, X and Y are each independently —OH, —OC₁₋₄alkyl, —N(R¹⁰⁰)(R¹⁰¹) or a bond covalently linked to L, provided one of A, B, X and Y is a bond covalently linked to L;
R¹⁰⁰ is H or C₁₋₃alkyl;
R¹⁰¹ is H, C₁₋₃alkyl or —(CH₂—CH₂—O)ₙᵣR¹⁰²;
R¹⁰² is H or Me;
nr is an integer from 1 to 16.

In yet another aspect, the present disclosure provides modified antibodies that be used to covalently link to a photosensitizer compound (e.g., compound of formula (I)) or a linker-photosensitizer compound (e.g., compound of formula (II)) described herein. In some embodiments the modified antibody is represented by the following formula:

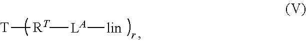

or a pharmaceutically acceptable salt thereof, wherein:
T is targeting agent;
R^{T} is a reactive group covalently linked to the targeting agent;
L^{A} is a spacer;
lin is a reactive group; and
r is an integer from 1 to 20.

In yet another aspect, the present disclosure provided a compound of formula (VI):

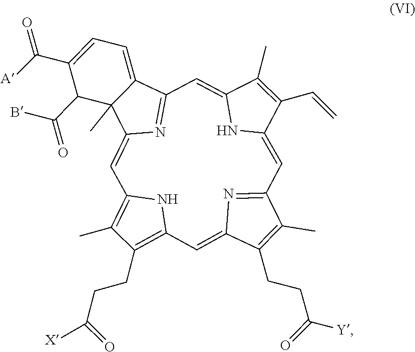

or a pharmaceutically acceptable salt thereof, wherein:
A′, B′, X′ and Y′ are each independently —OH, —OC₁₋₄alkyl, —N(R¹⁰⁰)(R¹⁰¹), or —Z—C₁₋₆allkylene-NH₂, provided one of A′, B′, X′ and Y′ is —Z—C₁₋₆allkylene-NH₂;
R¹⁰⁰ is H or C₁₋₃alkyl;
R¹⁰¹ is H, C₁₋₃alkyl or —(CH₂—CH₂—O)ₙᵣR¹⁰²;
R¹⁰² is H or Me;
nr is an integer from 1 to 16;
Z is —O— or —NR¹—; and
R¹ is H or C₁₋₃alkyl

In another aspect, the present disclosure provides a method of treating a subject having a disease or condition comprising:
a) administering to the subject an effective amount of the conjugate described herein, or a pharmaceutically acceptable salt thereof, or a pharmaceutical composition comprising the conjugate or a pharmaceutically acceptable salt thereof; and
b) after administering the conjugate or a pharmaceutically acceptable salt thereof or a pharmaceutical composition comprising the conjugate or a pharmaceutically acceptable salt thereof, illuminating a target region of the subject with a near infrared (NIR) light sufficient to activate the photosensitizer of the conjugate.

Also included in the present disclosure is a conjugate described herein, or a pharmaceutically acceptable salt thereof, or a pharmaceutical composition comprising the conjugate or a pharmaceutically acceptable salt thereof for use in a method for treating a subject having a disease or condition. The method comprises a) administering to the subject an effective amount of the conjugate described herein, or a pharmaceutically acceptable salt thereof, or a pharmaceutical composition comprising the conjugate or a pharmaceutically acceptable salt thereof; and b) after administering the conjugate or a pharmaceutically acceptable salt thereof or a pharmaceutical composition comprising the conjugate or a pharmaceutically acceptable salt thereof, illuminating a target region of the subject with a near infrared (NIR) light sufficient to activate the photosensitizer of the conjugate. The present disclosure also include the use of a conjugate described herein, or a pharmaceutically acceptable salt thereof, or a pharmaceutical composition comprising the conjugate or a pharmaceutically acceptable salt thereof for the manufacture of a medicament for treating a disease or condition. In some embodiments, the disease or condition to be treated by the present methods comprise cells or tissues expressing a target molecule that recognized by and/or bind to the targeting agent (e.g., an antibody or an antigen-binding fragment thereof) of the conjugates described herein.

The present also provides a method of imaging a cell or tissue having a target molecule in a subject comprising: a) administering to the subject a conjugate described herein, or a pharmaceutically acceptable salt thereof, or a pharmaceutical composition comprising the conjugate or a pharmaceutically acceptable salt thereof, and after administering the conjugate or a pharmaceutically acceptable salt thereof or a pharmaceutical composition comprising the conjugate or a pharmaceutically acceptable salt thereof, illuminating a target region of the subject with a near infrared (NIR) light sufficient to activate the photosensitizer of the conjugate, thereby providing an image of the presence of the target molecule associate with the cell or tissue. In some embodiments, the target region comprises the cell or tissue to be imaged or in the vicinity of the cell or tissue to be imaged.

In some embodiment, the target molecule is recognized by and/or bind to the targeting agent (e.g., an antibody or an antigen-binding fragment thereof) of the conjugates described herein.

### BRIEF DESCRIPTION OF THE DRAWINGS

FIG. 1A shows normalized UV-Vis spectra of benzoporphyrin analogs in Tris pH 8 and 50% DMSO.

FIG. 1B shows normalized UV-Vis spectra of benzoporphyrin analogs in Tris pH 8 and 50% DMSO.

FIG. 1C shows normalized UV-Vis spectra of benzoporphyrin analogs in Tris pH 8 and 50% DMSO.

FIG. 2A shows reaction oxygen species (ROS) generation of benzoporphyrin analogs at 10 J/cm² using Singlet Oxygen Sensor Green (SOSG) assay. The PBS-only baseline is indicated by a dotted line.

FIG. 2B shows reaction oxygen species (ROS) generation of benzoporphyrin analogs at 50 J/cm² using Singlet Oxygen Sensor Green (SOSG) assay. The PBS-only baseline is indicated by a dotted line.

FIG. 3A shows an assembly of non-specific, photosensitizer-antibody conjugates (PAC) using a non-cleavable linker. Specifically, the non-specific chemical acylation of amines (N-terminal or lysine) to construct photosensitizer antibody conjugates (PACs) via two-steps.

FIG. 3B shows the structures of clickable handles for conjugation of PACs via strain-promoted azide-alkyne click chemistry (SPAAC). The antibody is conjugated through amine acylation using NHS ester (1) to introduce an azide (denoted as “lin”). Each modified antibody is conjugated to photosensitizers (PS) with a complementary bicyclononyne clickable handle (compounds 2, 3 and 4; denoted as “ker”). The azide and bicyclononyne are click together to form a triazole (5, 6 and 7) in the final linker structure (denoted as “linker”). For the linker-photosensitizers and photosensitizers, only a single regioisomer is shown.

FIG. 4A shows assembly of non-specific, photosensitizer-antibody conjugates (PAC) using a cleavable linker. Specifically, the non-specific chemical acylation of amines (N-terminal or lysine) to construct photosensitizer antibody conjugates (PACs) via two-steps.

FIG. 4B shows the structures of clickable handles for conjugation of PACs via SPAAC. The antibody is conjugated through amine acylation using NHS ester to introduce a bicyclononyne clickable handle (denoted as “lin”). Each modified antibody is conjugated to photosensitizers (PS) with a complementary azide (denoted as “ker”). The bicyclononyne and azide are click together to form a triazole in the final linker structure (denoted as “linker”). For the linker-photosensitizers and photosensitizers, only a single regioisomer is shown.

FIGS. 5A-5C show characterization of photosensitizer-antibody conjugates (PACs) by non-specific conjugation using panitumumab and a non-cleavable linker.

FIG. 5A shows the reducing SDS-PAGE of panitumumab conjugates visualized by fluorescence imaging (excitation 455-485 nm and emission 675-720 nm). Lanes: 1) PS-0002 (a benzoporphyrin analog), 2) molecular weight standards, 3) panitumumab, 4) panitumumab incubated with NHS-PEG₆-azide (or NHS-azide) and PS-0002.

FIG. 5B shows the reducing SDS-PAGE of panitumumab conjugates visualized by Commassie staining. Lanes: 1) PS-0002 (a benzoporphyrin analog), 2) molecular weight standards, 3) panitumumab, 4) panitumumab incubated with NHS-PEG₆-azide (or NHS-azide) and PS-0002.

FIG. 5C shows normalized UV-vis spectra of panitumumab-benzoporphyrin analog conjugates. See FIG. 3B for structural details of the PACs.

FIGS. 6A-6C shows characterization of photosensitizer-antibody conjugates (PACs) by non-specific conjugation using anti-CD2 (clone: RPA-2.10) and cleavable linker.

FIG. 6A shows the reducing SDS-PAGE of anti-CD2 conjugates (PACs) visualized by fluorescence imaging (excitation 455-485 nm and emission 675-720 nm). Lanes: 1) PS-0025 (a benzoporphyrin analog), 2) molecular weight standards, 3) anti-CD2, 4) anti-CD2 incubated with NHS-PEG₅-BCN (or NHS-BCN), 5) anti-CD2 incubated with NHS-PEG₅-BCN (or NHS-BCN) and PS-0025.

FIG. 6B shows the reducing SDS-PAGE of anti-CD2 conjugates (PACs) visualized by Commassie staining. Lanes: 1) PS-0025 (a benzoporphyrin analog), 2) molecular weight standards, 3) anti-CD2, 4) anti-CD2 incubated with NHS-PEG₅-BCN (or NHS-BCN), 5) anti-CD2 incubated with NHS-PEG₅-BCN (or NHS-BCN) and PS-0025.

FIG. 6C shows normalized UV-vis spectra of RPA2.10-benzoporphyrin analog conjugates. See FIG. 4B for structural details of the PACs.

FIG. 7A shows phototoxicity of panitumumab conjugates constructed via non-specific conjugation. The conjugates were incubated in EGFR-expressing A-431 cells at 37° C. for 24 h at 50 nM. Data is normalized to a no treatment control. Light doses given were 0, 3, 10, 20, 30, 40 and 50 J/cm². FIGS. 5A-5C illustrate characterization of this panitumumab conjugate.

FIG. 7B shows phototoxicity of panitumumab conjugates constructed via non-specific conjugation. The conjugates were incubated in EGFR-expressing A-431 cells at 37° C. for 24 h at 25 nM. Data is normalized to a no treatment control. Light doses given were 0, 3, 10, 20, 30, 40 and 50 J/cm². FIGS. 5A-5C illustrate characterization of this panitumumab conjugate.

FIG. 8A shows phototoxicity of anti-CD2 conjugates constructed via non-specific conjugation. The conjugates were incubated in CD2-expressing Jurkat cells at 37° C. for 48 h at 50 nM. Data is normalized to a no treatment control. Light doses given were 0, 3, 10, 20, 30, 40 and 50 J/cm². FIGS. 6A-6C illustrates characterization of this anti-CD2 conjugate.

FIG. 8B shows phototoxicity of anti-CD2 conjugates constructed via non-specific conjugation. The conjugates were incubated in CD2-expressing Jurkat cells at 37° C. for 48 h at 25 nM. Data is normalized to a no treatment control. Light doses given were 0, 3, 10, 20, 30, 40 and 50 J/cm². FIGS. 6A-6C illustrates characterization of this anti-CD2 conjugate.

FIG. 9 shows reaction oxygen species (ROS) generation of benzoporphyrin analogs at 10 J/cm² and 50 J/cm² using Singlet Oxygen Sensor Green (SOSG) assay.

FIG. 10 shows phototoxicity of anti-CD2 conjugate constructed via non-specific conjugation. The conjugate was incubated at various concentrations with CD2-expressing HH cells at 37° C. for 24 h, and then given 100 J/cm² of NIR light. FIG. 6A-C illustrates characterization of this anti-CD2 conjugate.

FIG. 11A shows the assembly of non-specific, photosensitizer-antibody conjugates (PAC) using a cleavable linker. Specifically, the non-specific chemical acylation of amines (N-terminal or lysine) to construct photosensitizer antibody conjugates (PACs) via two-steps.

FIG. 11B shows the structures of clickable handles for conjugation of PACs via SPAAC. The antibody is conjugated through amine acylation using NHS ester to introduce an azide (clickable handle). Each modified antibody is conjugated to photosensitizers (PS) with a complementary bicyclononyne (a complimentary clickable handle). The bicyclononyne and azide are click together to form a triazole in the final linker structure. For the linker-photosensitizers and photosensitizers, only a single regioisomer is shown.

FIGS. 12A-12C show characterization of photosensitizer-antibody conjugates (PACs) by non-specific conjugation using panitumumab and a cleavable linker.

FIG. 12A shows the reducing SDS-PAGE of panitumumab conjugates visualized by fluorescence imaging (excitation 455-485 nm and emission 675-720 nm). Lanes: 1) molecular weight standards, 2) panitumumab, 3) PS-0011 (a benzoporphyrin analog), 4) panitumumab incubated with NHS-PEG6-azide (or NHS-azide), 5) panitumumab incubated with NHS-PEG6-azide (or NHS-azide) and PS-0011 (cleavable VertMA).

FIG. 12B shows the reducing SDS-PAGE of panitumumab conjugates visualized by Commassie staining. Lanes: 1) molecular weight standards, 2) panitumumab, 3) PS-0011 (a benzoporphyrin analog), 4) panitumumab incubated with NHS-PEG6-azide (or NHS-azide), 5) panitumumab incubated with NHS-PEG6-azide (or NHS-azide) and PS-0011 (cleavable VertMA).

FIG. 12C shows normalized UV-vis spectra of panitumumab-benzoporphyrin analog conjugates. See FIG. 11B for structural details of the PACs.

FIGS. 13A-13C show characterization of photosensitizer-antibody conjugates (PACs) by non-specific conjugation using panitumumab and a cleavable linker.

FIG. 13A shows the reducing SDS-PAGE of panitumumab conjugates visualized by fluorescence imaging (excitation 455-485 nm and emission 675-720 nm). Lanes: 1) PS-0020 (a benzoporphyrin analog), 2) molecular weight standards, 3) panitumumab, 4) panitumumab incubated with NHS-PEG₆-azide (or NHS-azide), 5) panitumumab incubated with NHS-PEG₆-azide (or NHS-azide) and PS-0020 (cleavable VertDA).

FIG. 13B shows the reducing SDS-PAGE of panitumumab conjugates visualized by Commassie staining. Lanes: 1) PS-0020 (a benzoporphyrin analog), 2) molecular weight standards, 3) panitumumab, 4) panitumumab incubated with NHS-PEG₆-azide (or NHS-azide), 5) panitumumab incubated with NHS-PEG₆-azide (or NHS-azide) and PS-0020 (cleavable VertDA).

FIG. 13C shows normalized UV-vis spectra of panitumumab-benzoporphyrin analog conjugates. See FIG. 11B for structural details of the PACs.

FIGS. 14A-14C show characterization of photosensitizer-antibody conjugates (PACs) by non-specific conjugation using panitumumab and a cleavable linker.

FIG. 14A shows the reducing SDS-PAGE of panitumumab conjugates visualized by fluorescence imaging (excitation 455-485 nm and emission 675-720 nm). Lanes: 1) molecular weight standards, 2) panitumumab, 3) PS-0019 (a benzoporphyrin analog), 4) panitumumab incubated with NHS-PEG6-azide (or NHS-azide), 5) panitumumab incubated with NHS-PEG6-azide (or NHS-azide) and PS-0019 (cleavable VertTA).

FIG. 14B shows the reducing SDS-PAGE of panitumumab conjugates visualized by Commassie staining. Lanes: 1) molecular weight standards, 2) panitumumab, 3) PS-0019 (a benzoporphyrin analog), 4) panitumumab incubated with NHS-PEG6-azide (or NHS-azide), 5) panitumumab incubated with NHS-PEG6-azide (or NHS-azide) and PS-0019 (cleavable VertTA).

FIG. 14C shows normalized UV-vis spectra of panitumumab-benzoporphyrin analog conjugates. See FIG. 11B for structural details of the PACs.

FIGS. 15A-15D show characterization of photosensitizer-antibody conjugates (PACs) by non-specific conjugation using panitumumab and a non-cleavable linker.

FIG. 15A shows the reducing SDS-PAGE of panitumumab conjugates visualized by fluorescence imaging (excitation 455-485 nm and emission 675-720 nm). Lanes: 1) molecular weight standards, 2) panitumumab, 3) PS-0002 (a benzoporphyrin analog), 4) panitumumab incubated with NHS-PEG₆-azide (or NHS-azide), 5) panitumumab incubated with NHS-PEG6-azide (or NHS-azide) and PS-0003 (non-cleavable VertDA), 6) panitumumab incubated with NHS-PEG6-azide (or NHS-azide) and PS-0002 (non-cleavable VertMA).

FIG. 15B shows the reducing SDS-PAGE of panitumumab conjugates visualized by Commassie staining. Lanes: 1) molecular weight standards, 2) panitumumab, 3) PS-0002 (a benzoporphyrin analog), 4) panitumumab incubated with NHS-PEG₆-azide (or NHS-azide), 5) panitumumab incubated with NHS-PEG6-azide (or NHS-azide) and PS-0003 (non-cleavable VertDA), 6) panitumumab incubated with NHS-PEG6-azide (or NHS-azide) and PS-0002 (non-cleavable VertMA).

FIG. 15C shows normalized UV-vis spectra of panitumumab-benzoporphyrin analog conjugates. See FIG. 3B for structural details of the PACs.

FIG. 15D shows normalized UV-vis spectra of panitumumab-benzoporphyrin analog conjugates. See FIG. 3B for structural details of the PACs.

FIGS. 16A-16C show characterization of photosensitizer-antibody conjugates (PACs) by non-specific conjugation using panitumumab and a non-cleavable linker.

FIG. 16A shows reducing SDS-PAGE of panitumumab conjugates visualized by fluorescence imaging (excitation 455-485 nm and emission 675-720 nm). Lanes: 1) molecular weight standards, 2) panitumumab, 3) PS-0004 (a benzoporphyrin analog), 4) panitumumab incubated with NHS-PEG6-azide (or NHS-azide), 5) panitumumab incubated with NHS-PEG6-azide (or NHS-azide) and PS-0004 (non-cleavable VertTA).

FIG. 16B shows educing SDS-PAGE of panitumumab conjugates visualized by Commassie staining. Lanes: 1) molecular weight standards, 2) panitumumab, 3) PS-0004 (a benzoporphyrin analog), 4) panitumumab incubated with NHS-PEG6-azide (or NHS-azide), 5) panitumumab incubated with NHS-PEG6-azide (or NHS-azide) and PS-0004 (non-cleavable VertTA).

FIG. 16C shows normalized UV-vis spectra of panitumumab-benzoporphyrin analog conjugates. See FIG. 3B for structural details of the PACs.

FIG. 17A shows structure-activity relationship of cleavable panitumumab conjugates constructed via non-specific conjugation. The conjugates were incubated at various concentrations with EGFR-expressing A-431 cells at 37° C. for 24 h, and then given 100 J/cm² of NIR light. Data is normalized to an untreated control. A-431 cells that received the maximum 50 nM PAC without light irradiation are indicated by “No Light”.

FIG. 17B shows the chemical structures of the different PAC constructs. FIGS. 15A-15C and 16A-16C illustrate characterization of these panitumumab conjugates.

FIG. 18A shows structure-activity relationship of non-cleavable panitumumab conjugates constructed via non-specific conjugation. The conjugates were incubated at various concentrations with EGFR-expressing A-431 cells at 37° C. for 24 h, and then given 100 J/cm² of NIR light. Data is normalized to an untreated control. A-431 cells that received the maximum 50 nM PAC without light irradiation are indicated by “No Light”.

FIG. 18B shows the chemical structures of the different PAC constructs. FIGS. 12A-12C, 13A-13C and 14A-14C illustrate characterization of these panitumumab conjugates.

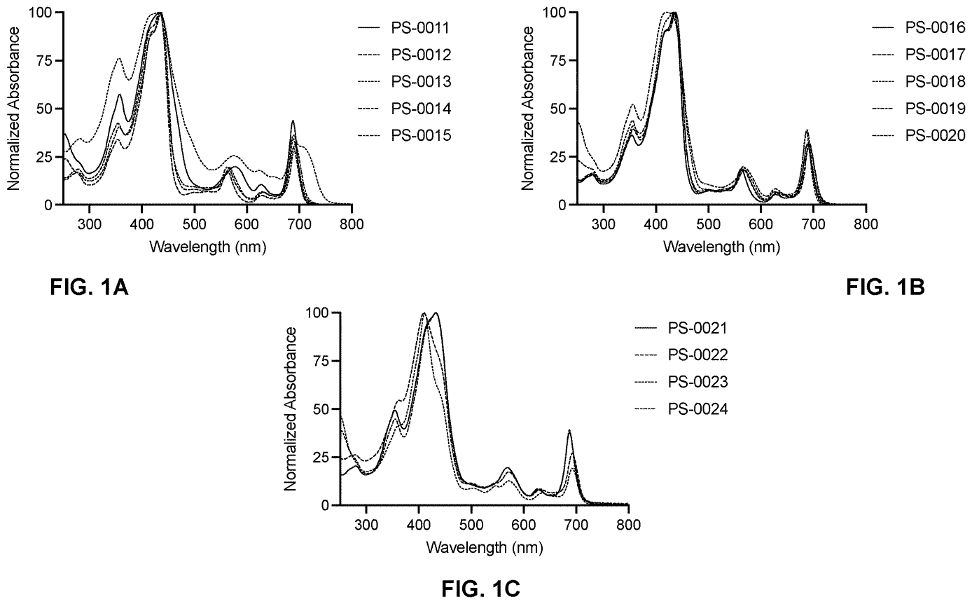

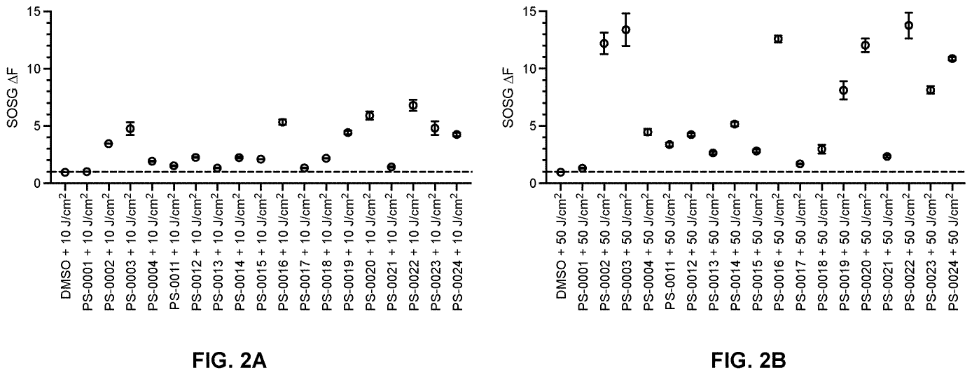

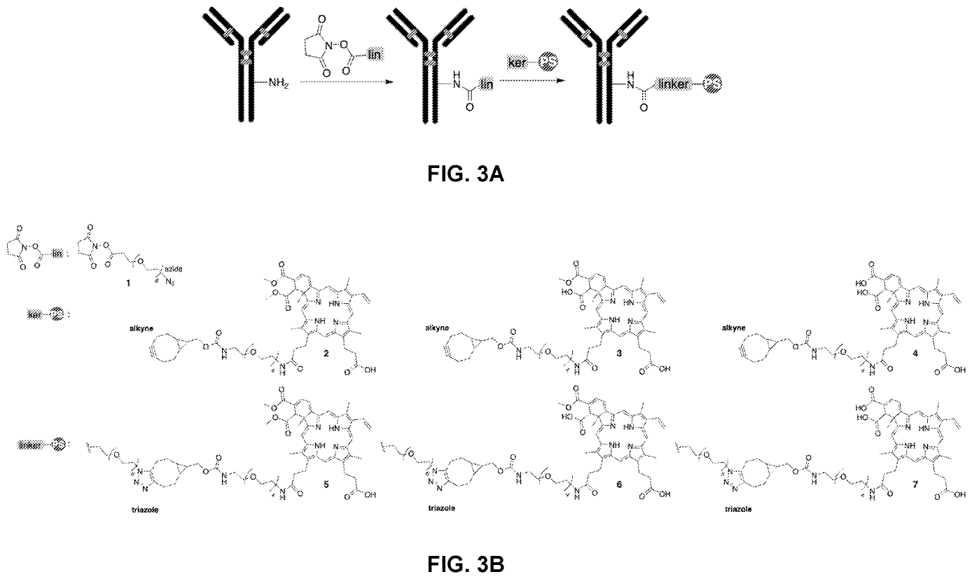

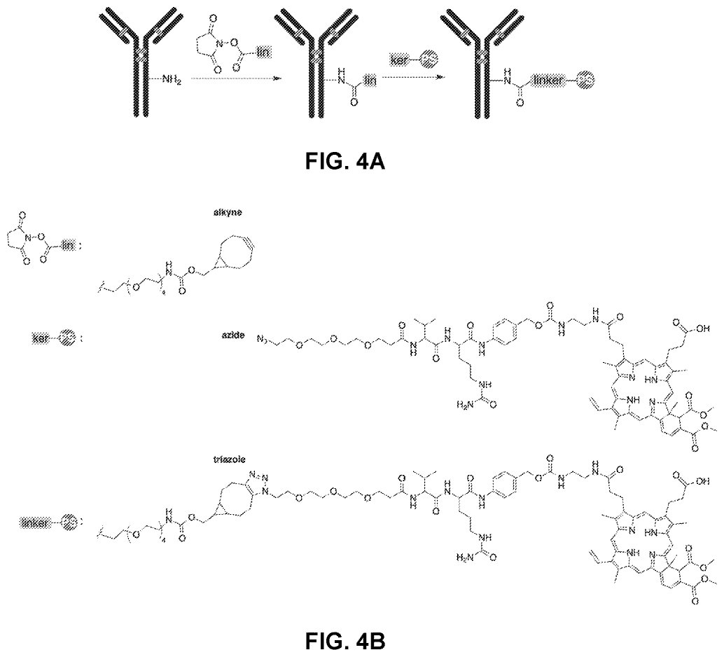

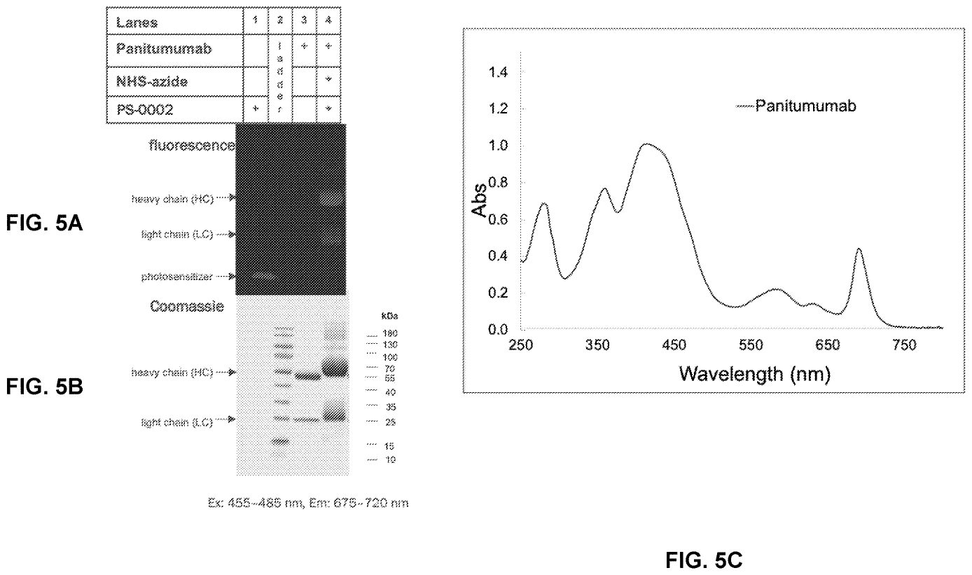

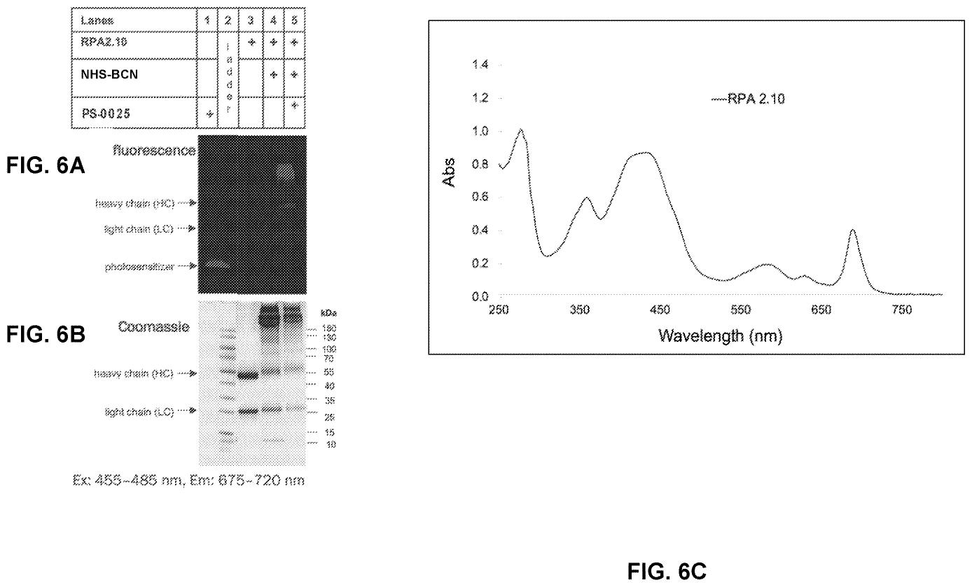

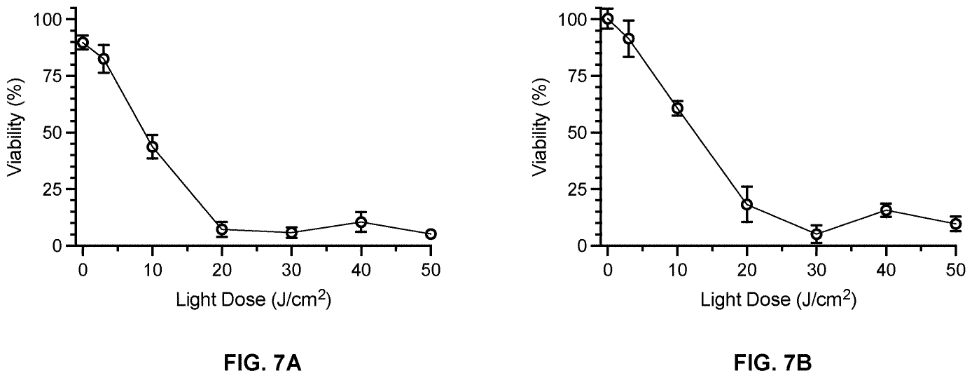

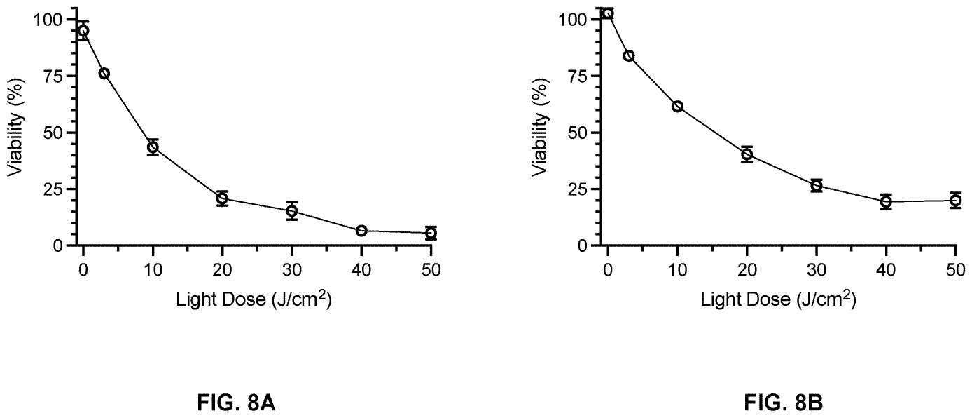

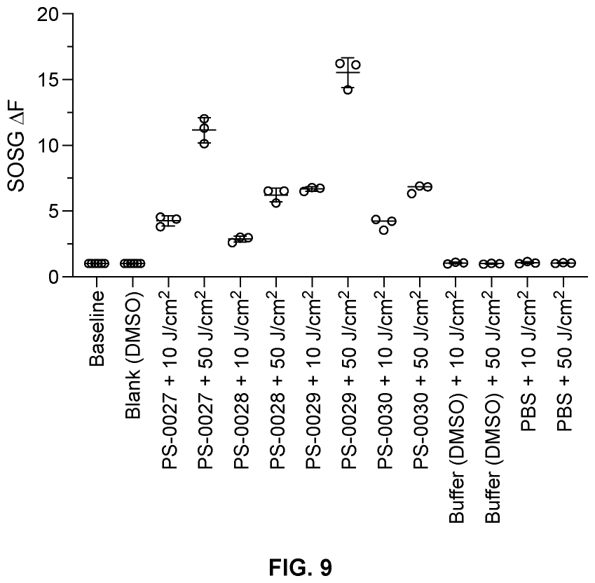

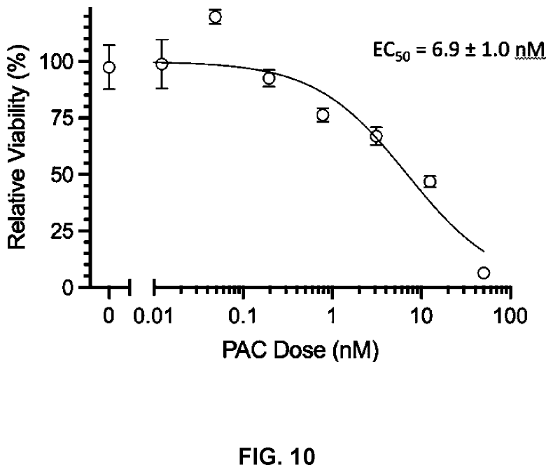

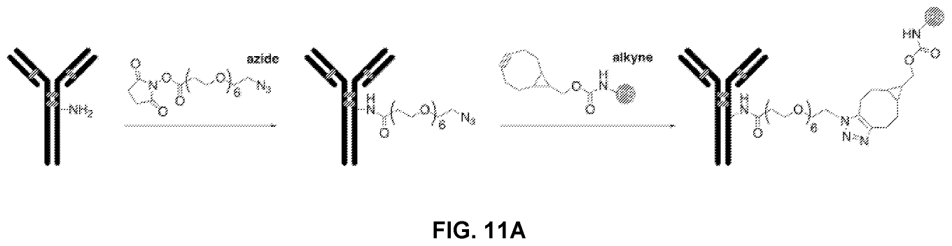

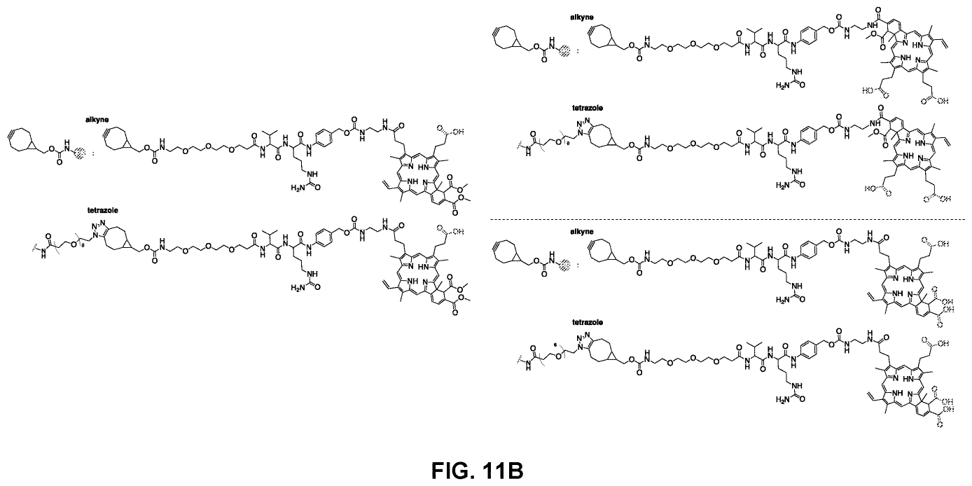

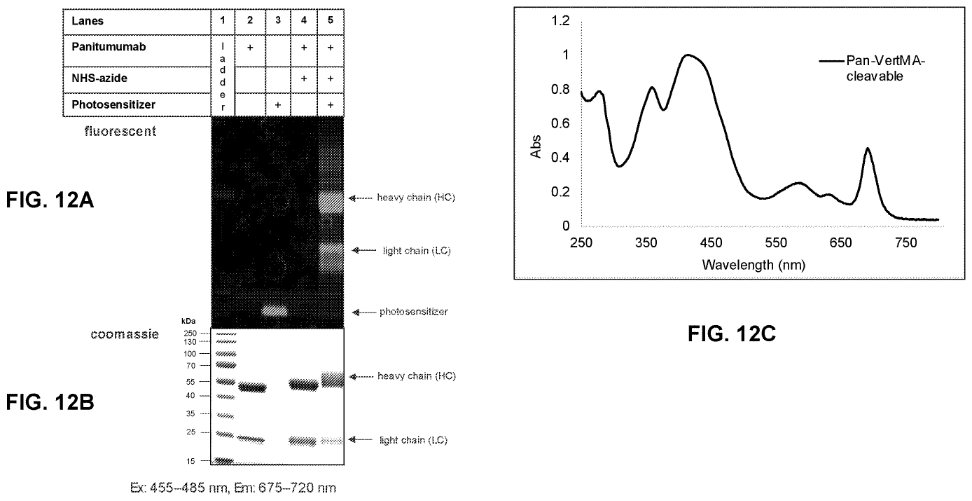

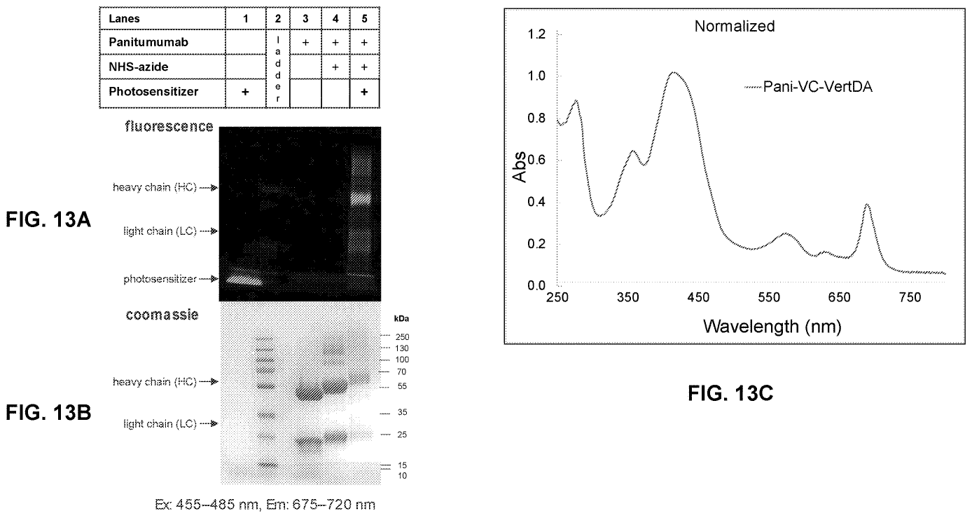

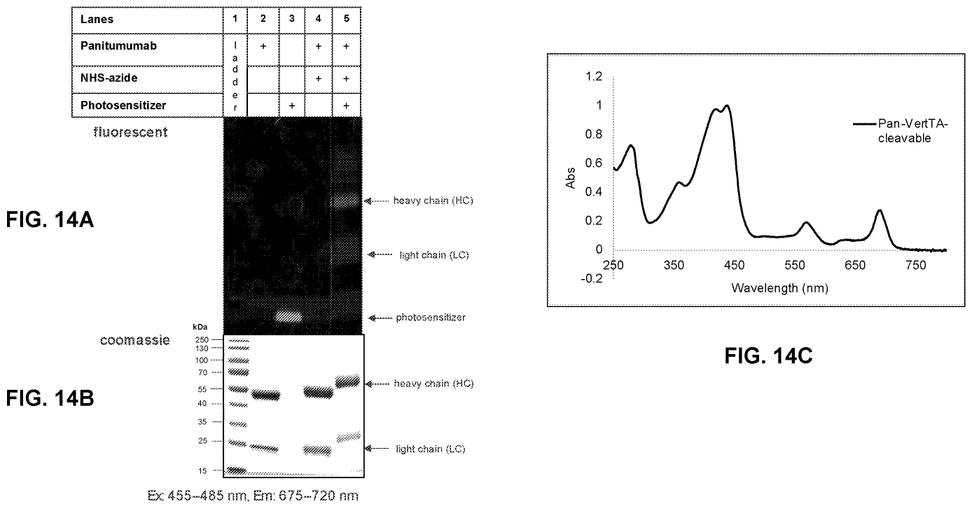

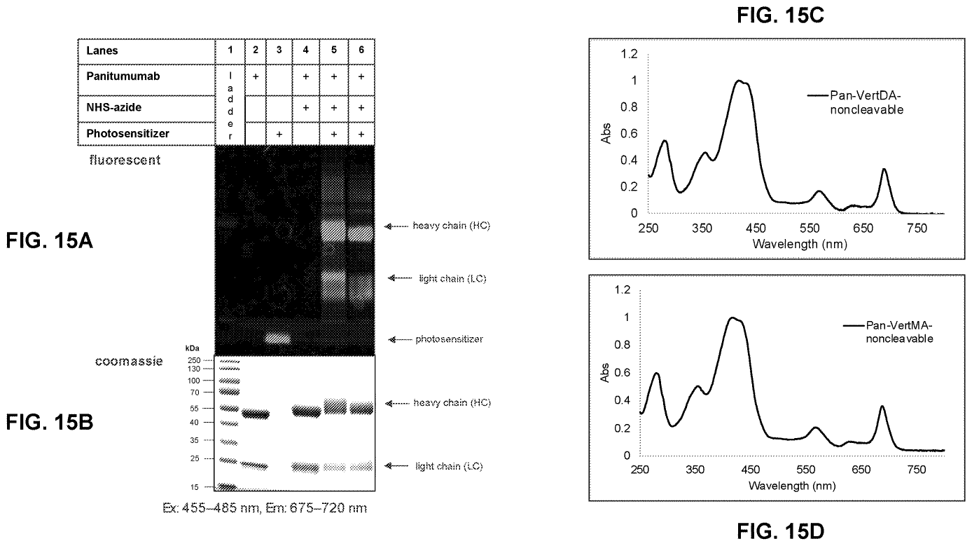

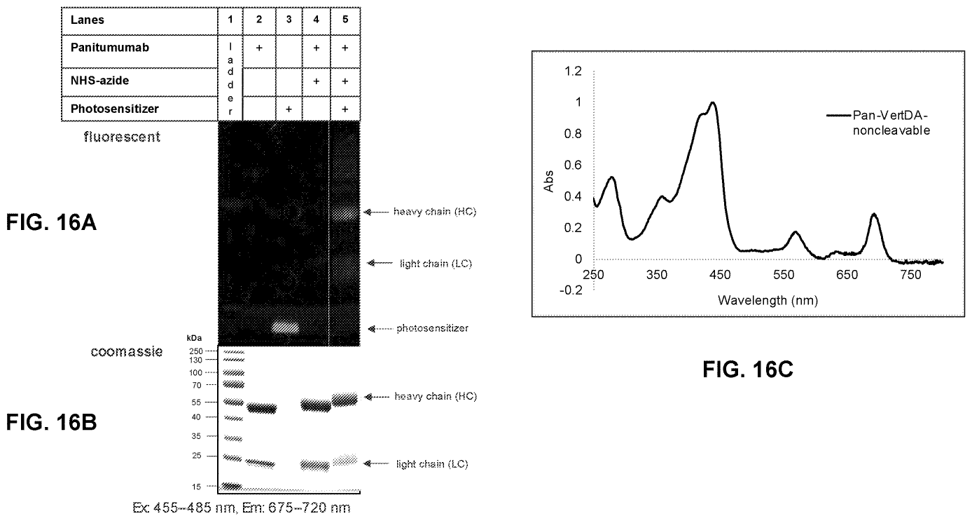

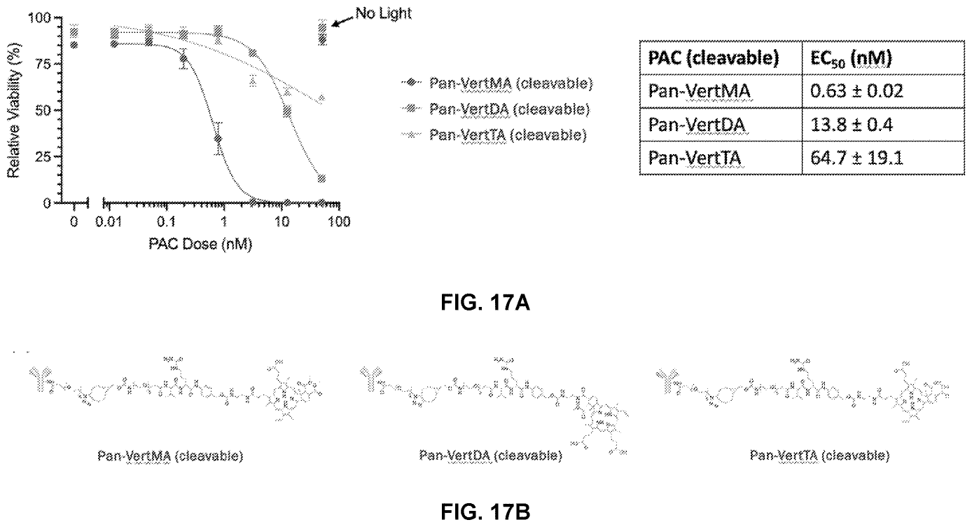

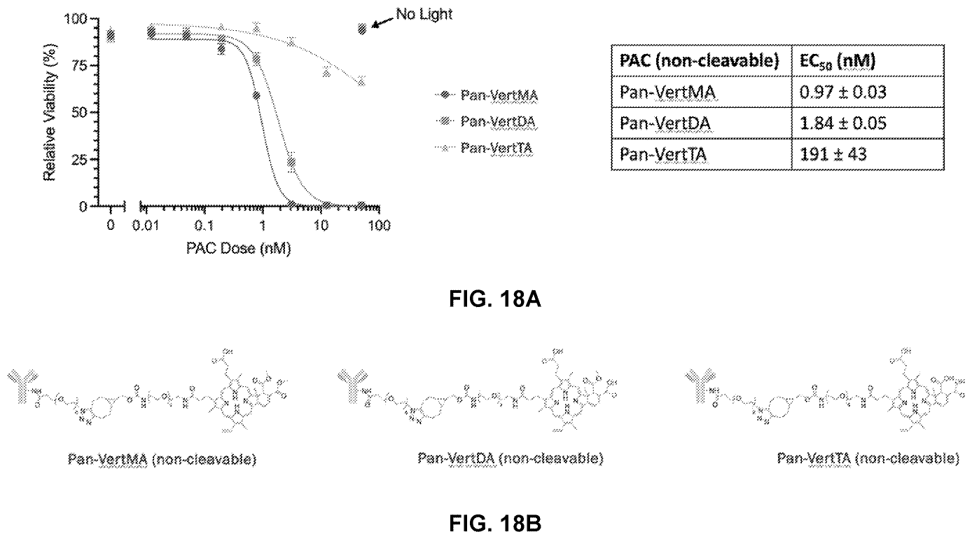

### DETAILED DESCRIPTION

The disclosed compositions and methods may be understood more readily by reference to the following detailed description taken in connection with the accompanying figures, which form a part of this disclosure.

Throughout this text, the descriptions refer to compositions and methods of using the compositions. Where the disclosure describes or claims a feature or embodiment associated with a composition, such a feature or embodiment is equally applicable to the methods of using the composition. Likewise, where the disclosure describes or claims a feature or embodiment associated with a method of using a composition, such a feature or embodiment is equally applicable to the composition.

When a range of values is expressed, it is inclusive of their endpoints. When values are expressed as approximations, by use of the antecedent “about,” it will be understood that the particular value forms another embodiment. Reference to a particular numerical value includes at least that particular value, unless the context clearly dictates otherwise. All references cited herein are incorporated by reference for any purpose. Where a reference and the specification conflict, the specification will control.

Unless the context of a description indicates otherwise, e.g., in the absence of symbols indicating specific point(s) of connectivity, when a structure or fragment of a structure is drawn, it may be used on its own or attached to other components of a PAC, and it may do so with any orientation, e.g., with the antibody attached at any suitable attachment point to a chemical moiety such as a linker-drug. Where indicated, however, components of a PAC are attached in the orientation shown in a given formula.

It is to be appreciated that certain features of the disclosed compositions and methods, which are, for clarity, described herein in the context of separate embodiments, may also be provided in combination in a single embodiment. Conversely, various features of the disclosed compositions and methods that are, for brevity, described in the context of a single embodiment, may also be provided separately or in any sub-combination.

### Definitions

The term “herein” means the entire application.

Unless otherwise defined herein, scientific and technical terms used in this application shall have the meanings that are commonly understood by those of ordinary skill in the art to which this present disclosure belongs. Generally, nomenclature used in connection with the compounds, composition and methods described herein, are those well-known and commonly used in the art.

It should be understood that any of the embodiments described herein, including those described under different aspects of the present disclosure and different parts of the specification (including embodiments described only in the Examples) can be combined with one or more other embodiments of the present disclosure, unless explicitly disclaimed or improper. Combination of embodiments are not limited to those specific combinations claimed via the multiple dependent claims.

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

As used herein, the singular forms “a,” “an,” and “the” include plural forms unless the context clearly dictates otherwise. The terms “comprising”, “having”, “including”, and “containing” are to be construed as open terms (i.e., meaning “including but not limited to”) unless otherwise noted. Additionally whenever “comprising” or another open-ended term is used in an embodiment, it is to be understood that the same embodiment can be more narrowly claimed using the intermediate term “consisting essentially of” or the closed term “consisting of”.

The term “about” or “approximately,” when used in the context of numerical values and ranges, refers to values or ranges that approximate or are close to the recited values or ranges such that the embodiment may perform as intended, as is apparent to the skilled person from the teachings contained herein. In some embodiments, about means plus or minus 20%, 15%, 10%, 5%, 1%, 0.5%, or 0.1% of a numerical amount.

As used herein, the term “benzoporphyrin analogs” or “BP analogs” refers to compounds that have the benzoporphyrin core structure:

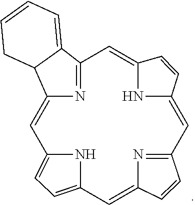 [SMILES: C1=CCC2C(=C1)c1cc3ccc(cc4nc(cc5ccc(cc2n1)[nH]5)C=C4)[nH]3]

The benzoporphyrin core can be substituted or unsubstituted, and/or fused with one or more ring structures.

The term “alkyl” or “linear or branched alkyl” as used herein refers to a saturated linear or branched monovalent hydrocarbon radical. In preferred embodiments, a straight chain or branched chain alkyl has thirty or fewer carbon atoms (e.g., C₁-C₃₀ for straight chain alkyl group and C₃-C₃₀ for branched alkyl), and more preferably twenty or fewer carbon atoms. Even more preferably, the straight chain or branched chain alkyl has ten or fewer carbon atoms (i.e., C₁-C₁₀ for straight chain alkyl group and C₃-C₁₀ for branched alkyl). In other embodiments, the straight chain or branched chain alkyl has six or fewer carbon atoms (i.e., C₁-C₆ for straight chain alkyl group or C₃-C₆ for branched chain alkyl). Examples of alkyl include, but are not limited to, methyl, ethyl, 1-propyl, 2-propyl, 1-butyl, 2-methyl-1-propyl, —CH₂CH(CH₃)₂), 2-butyl, 2-methyl-2-propyl, 1-pentyl, 2-pentyl 3-pentyl, 2-methyl-2-butyl, 3-methyl-2-butyl, 3-methyl-1-butyl, 2-methyl-1-butyl, 1-hexyl), 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, 1-heptyl, 1-octyl, and the like. Moreover, the term “alkyl” as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. As used herein, (Cₓ-Cₓₓ)alkyl or Cₓ₋ₓₓalkyl means a linear or branched alkyl having x-xx number of carbon atoms.

The term “alkylene” as used herein refers to a saturated linear or branched divalent hydrocarbon radical. In preferred embodiments, a straight chain or branched chain alkylene has thirty or fewer carbon atoms (e.g., C₁-C₃₀ for straight chain alkylene group and C₃-C₃₀ for branched alkylene), and more preferably twenty or fewer carbon atoms. Even more preferably, the straight chain or branched chain alkylene has ten or fewer carbon atoms (i.e., C₁-C₁₀ for straight chain alkylene group and C₃-C₁₀ for branched alkylene). In other embodiments, the straight chain or branched chain alkylene has six or fewer carbon atoms (i.e., C₁-C₆ for straight chain alkylene group or C₃-C₆ for branched chain alkylene). As used herein, (Cₓ-Cₓₓ)alkylene or Cₓ₋ₓₓalkylene means a linear or branched alkylene having x-xx number of carbon atoms.

The term “compound” is intended to include compounds for which a structure or formula or any analog thereof has been disclosed in the present disclosure or a structure or formula or any analog thereof that has been incorporated by reference. Also included in the present disclosure is stereoisomers, regioisomers (or position isomers), tautomers, solvates, metabolites, salts (e.g., pharmaceutically acceptable salts) of a compound of all the formulae disclosed in the present disclosure.

The term “conjugate” as used herein refers to a compound described herein or a analog thereof that is linked to a target binding agent.

The term “chiral” refers to molecules that have the property of non-superimposability of the mirror image partner, while the term “achiral” refers to molecules that are superimposable on their mirror image partner.

The term “stereoisomer” refers to compounds that have identical chemical constitution and connectivity, but different orientations of their atoms in space that cannot be interconverted by rotation about single bonds.

The term “diastereomer” refers to a stereoisomer with two or more centers of chirality and whose molecules are not mirror images of one another. Diastereomers have different physical properties, e.g. melting points, boiling points, spectral properties, and reactivities.

Mixtures of diastereomers can separate under high resolution analytical procedures such as crystallization, electrophoresis and chromatography.

The term “enantiomers” refer to two stereoisomers of a compound that are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds,” John Wiley & Sons, Inc., New York, 1994. The compounds of the present disclosure can contain asymmetric or chiral centers, and therefore exist in different stereoisomeric forms. It is intended that all stereoisomeric forms of the compounds of the present disclosure, including but not limited to, diastereomers, enantiomers and atropisomers, as well as mixtures thereof such as racemic mixtures, form part of the present disclosure. Many organic compounds exist in optically active forms, i.e., they have the ability to rotate the plane of plane-polarized light.

In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

The term “tautomer” or “tautomeric form” refers to structural isomers of different energies that are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

The term “regioisomer” refers to structural isomers that differ only on the position of a functional group or substituent. When a compound is depicted by a structure, unless otherwise specied, it also includes possible regioisomers of the compound. For example, for the photosensitizer compound of formula (I):

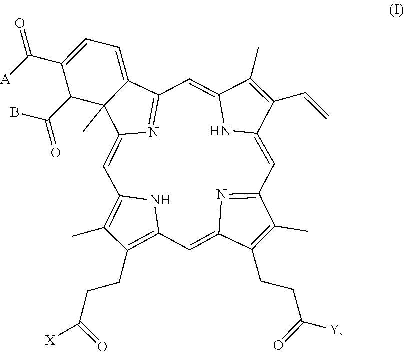

the groups A, B, X and Y can be switched to form a regioisomer. In some embodiments, the groups A and B can be switched to form a regioisomer. In some embodiments, the groups X and Y can be switched to form a regioisomer. In some embodiments, the groups A and X or the groups A and Y can be switched to form a regioisomer. In some embodiments, the groups B and X or the groups B and Y can be switched to form a regioisomer. For example, for compound of formula (IA), it includes the following two regioisomers:

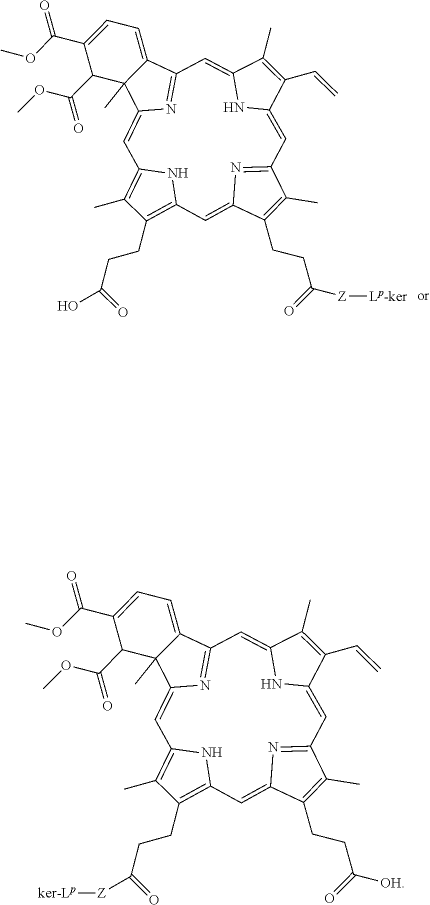

In another example, for the compound represented by the following formula:

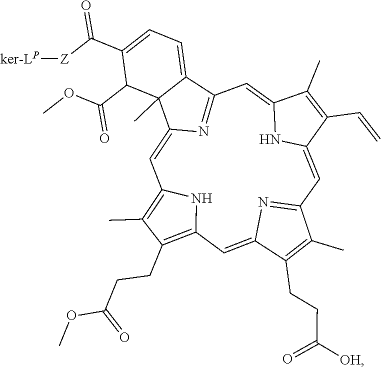

it also includes the following regioisomer:

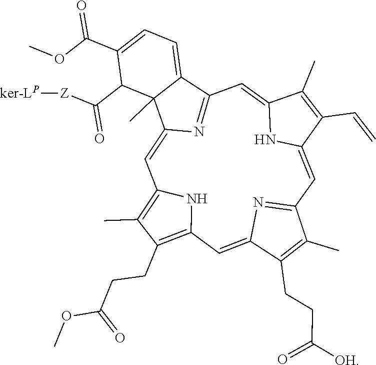

In another example, for the compound represented by the following formula:

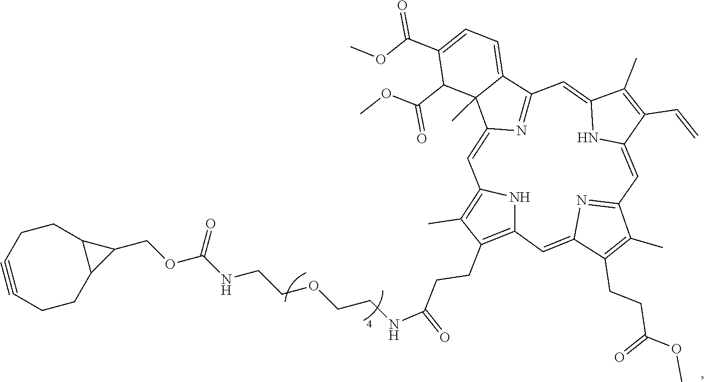

it also includes the following regioisomers:

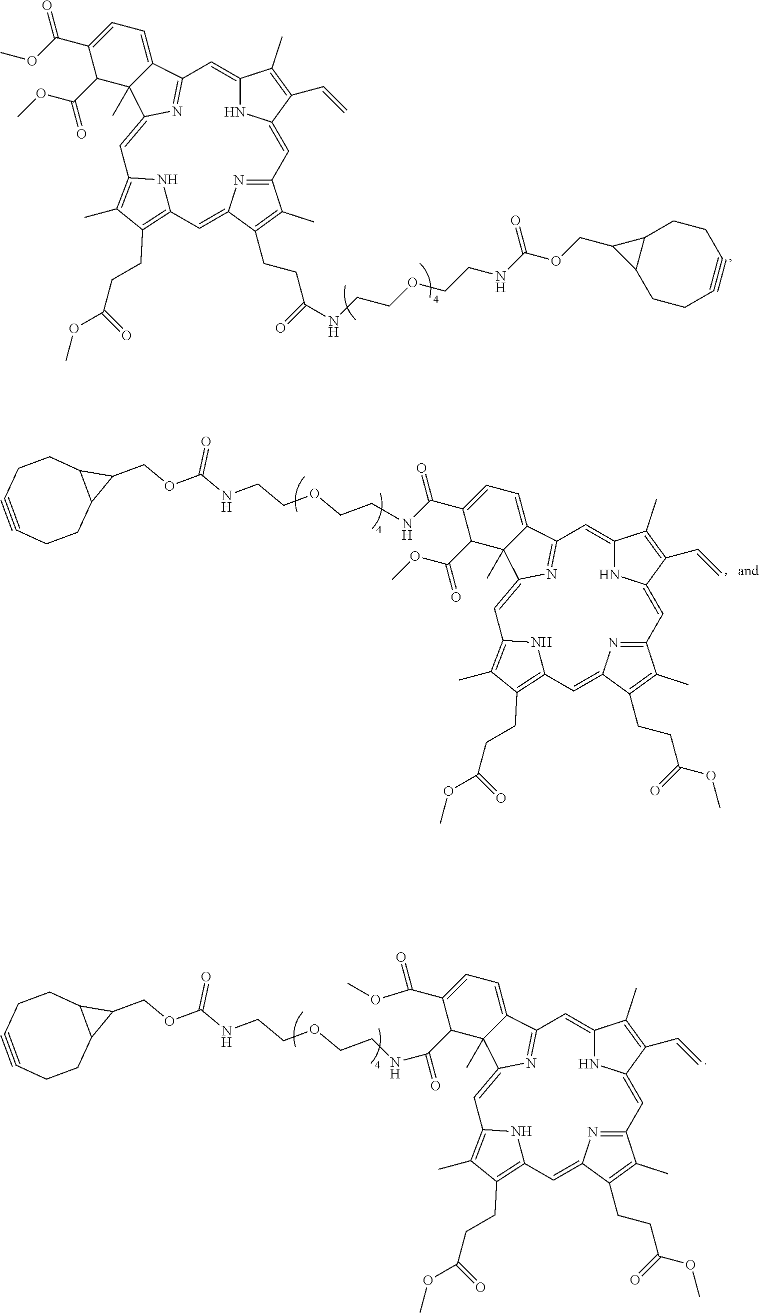

The term “pharmaceutically acceptable salt” as used herein, refers to pharmaceutically acceptable organic or inorganic salts of a compound or conjugate of the present disclosure. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt can involve the inclusion of another molecule such as an acetate ion, a succinate ion or other counter ion. The counter ion can be any organic or inorganic moiety that stabilizes the charge on the parent compound. Furthermore, a pharmaceutically acceptable salt can have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

If the compound of the present disclosure is a base, the desired pharmaceutically acceptable salt can be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of the present disclosure is an acid, the desired pharmaceutically acceptable salt can be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

As used herein, the term “solvate” means a compound that further includes a stoichiometric or non-stoichiometric amount of solvent such as water, isopropanol, acetone, ethanol, methanol, DMSO, ethyl acetate, acetic acid, and ethanolamine dichloromethane, 2-propanol, or the like, bound by non-covalent intermolecular forces. Solvates or hydrates of the compounds are readily prepared by addition of at least one molar equivalent of a hydroxylic solvent such as methanol, ethanol, 1-propanol, 2-propanol or water to the compound to result in solvation or hydration of the imine moiety.

The phrase “pharmaceutically acceptable” indicates that the substance or composition must be compatible chemically and/or toxicologically, with the other ingredients comprising a formulation, and/or the mammal being treated therewith.

The term “reactive ester” refers to an ester having an easily displaceable leaving group that can readily react with an amine group to form an amide bond. Examples of reactive esters include, but are not limited to, N-hydroxysuccinimide ester, N-hydroxy sulfosuccinimide ester, nitrophenyl (e.g., 2 or 4-nitrophenyl) ester, dinitrophenyl (e.g., 2,4-dinitrophenyl) ester, sulfo-tetraflurophenyl (e.g., 4 sulfo-2,3,5,6-tetrafluorophenyl) ester, or pentafluorophenyl ester.

The term “reactive group” refers to a group that can react with a moiety located on another molecule, such as the targeting agent or the photosensitizer compound, to form a covalent bond. The reactive group includes, but is not limited to an amine reactive group, a thiol reactive group, an azide and an alkyne.

The term “amine reactive group” refers to a group that can react with an amine group to form a covalent bond. Exemplary amine reactive groups include, but are not limited to, reactive ester groups, acyl halides, sulfonyl halide, imidoester, or a reactive thioester groups. In certain embodiments, the amine reactive group is a reactive ester group. In one embodiment, the amine reactive group is a N-hydroxysuccinimide ester or a N-hydroxy sulfo-succinimide ester.

The term “thiol-reactive group” refers to a group that can react with a thiol (—SH) group to form a covalent bond. Exemplary thiol-reactive groups include, but are not limited to, maleimide, haloacetyl, aloacetamide, vinyl sulfone, vinyl sulfonamide or vinyl pyridine. In one embodiment, the thiol-reactive group is maleimide.

The term “spacer” as defined herein refers to a moiety that connects two groups, such as a targeting agent and a photosensitizer compound or two components of the conjugates described herein, together. Typically, the spacer is substantially inert under conditions for which the two groups it is connecting are linked. A spacer can comprise two reactive groups, one at each ends of a linker moiety, such that one reactive group can be first reacted with the photosensitizerc compound to provide a compound bearing the spacer moiety and a second reactive group, which can then react with a targeting agent. Alternatively, one end of the spacer can be first reacted with the targeting agent to provide a targeting agent bearing a spacer moiety and a second reactive group, which can then react with a photosensitizer compound. The spacer can be “cleavable spacer”, which contains a chemical bond that allows for the release of the photosensitizer at a particular site. Suitable chemical bonds are well known in the art and include disulfide bonds, thioether bonds, pH labile bonds, ROS cleavable bond, photolabile bonds, enzyme-cleavable bonda, including peptidase labile bonds and esterase labile bonds, (see, for example, Sun et al. Bioconjugate Chem. 2020, 31, 1012-1024; Tong et al. Molecules 2021, 26, 5847; Alfaro et al., Anal. Chem. 2008, 80, 3882-3889; Chari et al., Angew. Chem. Int. Ed. 2014, 53, 3796-3827; Liang et al. Bioengineering & Translational Medicine 2016; 1: 239-251; Subramaniyan et al Photochemistry and Photobiology, 2020, 96: 668-679; Peterson et al J. Am. Chem. Soc. 2018, 140, 7343-7346; Vickerman et al Nature Review Chemistry, 2021, 5, 816-834; Nani et al Angew. Chem. 2015, 127, 13839-13842; Weinstein et al Chem. Rev. 2020, 120, 13135-13272; Sun et al. Bioconjugate Chem. 2020, 31, 1012-1024; all of these references are incorporated herein by reference). Preferred are disulfide bonds, thioether and peptidase labile bonds. Other spacers that can be used in the present disclosure include non-cleavable spacers, such as those described in Chari et al., Angew. Chem. Int. Ed. 2014, 53, 3796-3827.

The term “connection moiety” refers to a moiety formed by reaction of a first reactive group and a second reactive group. For example, the first reactive group and the second reactive group can be an an amine and a reactive ester and the connection moiety is an amide formed by reacting an amine and a reactive ester group.

The term “hydrophilic moiety” refers to a moiety that has hydrophilic properties, which increase the aqueous solubility of the photosensitizer compound or linker-photosensitizer compound. Examples of hydrophilic moiety include, but are not limited, to polyethylene glycols, polyalkylene glycols, sugars, or oligosaccharides. In some embodiments, the hydrophiclic moiety is polyethylene glycol (PEG).

The term “self-immolative group” refers to a moiety that will allow for release of the photosensitizer compound when a remote site is activated. In certain embodiments, the group comprises a p-aminobenzyl unit. In some such embodiments, a p-aminobenzyl alcohol is attached to an amino acid unit via an amide bond, and a carbamate, methylcarbamate, or carbonate is made between the benzyl alcohol and the drug (Hamann et al. (2005) Expert Opin. Ther. Patents (2005) 15:1087-1103). In some embodiments, the group comprises p-aminobenzyloxycarbonyl (PAB). Other examples of self-immolative groups include, but are not limited to, aromatic moieties that are electronically similar to the PAB group, such as 2-aminoimidazol-5-methanol derivatives (U.S. Pat. No. 7,375,078; Hay et al. (1999) Bioorg.

Med. Chem. Lett. 9:2237) and ortho- or para-aminobenzylacetals. In some embodiments, spacers can be used that undergo cyclization upon amide bond hydrolysis, such as substituted and unsubstituted 4-aminobutyric acid amides (Rodrigues et al (1995) Chemistry Biology 2:223), appropriately substituted bicyclo[2.2.1] and bicyclo[2.2.2] ring systems (Storm et al (1972) J. Amer. Chem. Soc. 94:5815) and 2-aminophenylpropionic acid amides (Amsberry, et al (1990) J. Org. Chem. 55:5867). Linkage of a photosensitizer compound to the α-carbon of a glycine residue is another example of a self-immolative group that may be useful in PAC (Kingsbury et al (1984) J. Med. Chem. 27:1447).

The term “amino acid” refers to naturally occurring amino acids or non-naturally occurring amino acid. In some embodiments, the amino acid is represented by NH₂—C(R^{aa′}R^{aa})—C(═O)OH, wherein R^{aa} and R^{aa′} are each independently H, an optionally substituted linear, branched or cyclic alkyl, alkenyl or alkynyl having 1 to 10 carbon atoms, aryl, heteroaryl or heterocyclyl or R^{aa} and the N-terminal nitrogen atom can together form a heterocyclic ring (e.g., as in proline). The term “amino acid residue” refers to the corresponding residue when one hydrogen atom is removed from the amine and/or carboxy end of the amino acid, such as —NH—C(R^{aa′}R^{aa})—C(═O)—.

The term “peptide” refers to short chains of amino acid monomers linked by peptide (amide) bonds. In some embodiments, the peptides contain 2 to 20 amino acid residues. In other embodiments, the peptides contain 2 to 10 or 2 to 8 amino acid residues. In yet other embodiments, the peptides contain 2 to 5 amino acid residues. As used herein, when a peptide is a portion of a cytotoxic agent or a linker described herein represented by a specific sequence of amino acids, the peptide can be connected to the rest of the cytotoxic agent or the linker in both directions.

The term “antibody” is used refer to an immunoglobulin molecule that recognizes and specifically binds to a target molecule, such as a protein, polypeptide, carbohydrate, polynucleotide, lipid, or combinations of the foregoing through at least one antigen recognition site within the variable region of the immunoglobulin molecule. An antibody can be polyclonal or monoclonal, multiple or single chain, or an intact immunoglobulin, and may be derived from natural sources or from recombinant sources. An antibody can be a monoclonal antibody, human antibody, humanized antibody, camelised antibody, or chimeric antibody. The antibodies can be of any isotype (e.g., IgG, IgE, IgM, IgD, IgA and IgY), class (e.g., IgG1, IgG2, IgG3, IgG4, IgAQ1 and IgA2), or subclass.

In some embodiments, an antibody is a non-naturally occurring antibody. In some embodiments, an antibody is purified from natural components. In some embodiments, an antibody is recombinantly produced. In some embodiments, an antibody is produced by a hybridoma.

The term “antibody fragment” or “antigen-binding fragment” refers to a portion of an intact antibody and refers to the antigenic determining variable regions of an intact antibody. Examples of antibody fragments include, but are not limited to, Fab, Fab′, F(ab′)₂, and Fᵥ fragments, linear antibodies, single chain antibodies, and multispecific antibodies (e.g., bispecific, biparatopic) formed from antibody fragments. The term “antigen-binding fragment” of an antibody includes one or more fragments of an antibody that retain the ability to specifically bind to an antigen. It has been shown that the antigen-binding function of an antibody can be performed by certain fragments of a full-length antibody. Examples of binding fragments encompassed within the term “antigen-binding fragment” of an antibody include (without limitation): (i) an Fab fragment, a monovalent fragment consisting of the V_{L}, V_{H}, C_{L}, and C_{H1} domains (e.g., an antibody digested by papain yields three fragments: two antigen-binding Fab fragments, and one Fc fragment that does not bind antigen); (ii) a single chain Fab (scFab), a fragment consisting of the V_{L}, V_{H}, C_{L}, and C_{H}1 domains, wherein the C_{L} and V_{H} domains are linked via a linker peptide; (iii) a F(ab′)₂ fragment, a bivalent fragment comprising two Fab fragments linked by a disulfide bridge at the hinge region (e.g., an antibody digested by pepsin yields two fragments: a bivalent antigen-binding F(ab′)₂ fragment, and a pFc′ fragment that does not bind antigen) and its related F(ab′) monovalent unit; (iv) a F_{d} fragment consisting of the V_{H} and C_{H1} domains (i.e., that portion of the heavy chain which is included in the Fab); (v) a Fᵥ fragment consisting of the V_{L} and V_{H} domains of a single arm of an antibody, and the related disulfide linked Fᵥ; (vi) a dAb (domain antibody) or sdAb (single domain antibody) fragment (Ward et al., Nature 341:544-546, 1989), which consists of a V_{H} domain; (vii) an isolated complementarity determining region (CDR); (viii) a single chain variable fragment (scFv), a fragment consisting of a V_{H} and V_{L} domain, connected via a linker peptide; and (ix) a tetravalent antibody, which may include various formats (structures) whereby the antibody comprises 4 antigen binding sites.

The term “monoclonal antibody,” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256:495, or may be made by recombinant DNA methods (see, e.g., U.S. Pat. No. 4,816,567). Monoclonal antibodies may also be isolated from phage antibody libraries using the techniques described in Clackson et al. (1991) Nature 352:624-8, and Marks et al. (1991) J Mol Biol. 222:581-97, for example. The term also includes preparations of antibody molecules of single molecular composition. A monoclonal antibody composition displays a single binding specificity and affinity for a particular epitope.

The monoclonal antibodies described herein can be non-human, human, or humanized. The term specifically includes “chimeric” antibodies, in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity.

The term “human antibody,” as used herein, refers an antibody produced by a human or an antibody having an amino acid sequence of an antibody produced by a human. The term includes antibodies having variable regions in which both the framework and CDR regions are derived from sequences of human origin. Furthermore, if the antibody contains a constant region, the constant region is also derived from such human sequences, e.g., human germline sequences, or mutated versions of human germline sequences or antibody containing consensus framework sequences derived from human framework sequences analysis, for example, as described in Knappik et al. ((2000) J Mol Biol. 296(1):57-86). The human antibodies of the present disclosure may include amino acid residues not encoded by human sequences (e.g., mutations introduced by random or site-specific mutagenesis in vitro or by somatic mutation in vivo, or a conservative substitution to promote stability or manufacturing). However, the term “human antibody,” as used herein, is not intended to include antibodies in which CDR sequences derived from the germline of another mammalian species, such as a mouse, have been grafted onto human framework sequences.

The term “recombinant human antibody,” as used herein, refers to a human antibody that is prepared, expressed, created, or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In some embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “chimeric antibody,” as used herein, refers to antibodies wherein the amino acid sequence of the immunoglobulin molecule is derived from two or more species. In some instances, the variable regions of both heavy and light chains correspond to the variable regions of antibodies derived from one species with the desired specificity, affinity, and activity while the constant regions are homologous to antibodies derived from another species (e.g., human) to minimize an immune response in the latter species.

As used herein, the term “humanized antibody” refers to forms of antibodies that contain sequences from non-human (e.g., murine) antibodies as well as human antibodies. Such antibodies are a type of chimeric antibody which contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the framework (FR) regions are those of a human immunoglobulin sequence. The humanized antibody optionally also will comprise at least a portion of an immunoglobulin constant region (Fc), typically that of a human immunoglobulin. The humanized antibody can be further modified by the substitution of residues, either in the Fv framework region and/or within the replaced non-human residues to refine and optimize antibody specificity, affinity, and/or activity.

As used herein, the terms “polypeptide,” and “protein” are used interchangeably to refer to a polymer of amino acid residues. The terms encompass amino acid polymers comprising two or more amino acids joined to each other by peptide bonds, amino acid polymers in which one or more amino acid residues is an artificial chemical mimetic of a corresponding naturally-occurring amino acid, as well as naturally-occurring amino acid polymers and non-naturally-occurring amino acid polymers. The terms include, for example, biologically active fragments, substantially homologous polypeptides, oligopeptides, homodimers, heterodimers, variants of polypeptides, modified polypeptides, derivatives, analogs, fusion proteins, among others. The terms also include natural peptides, recombinant peptides, synthetic peptides, or a combination thereof. Unless otherwise indicated, a particular polypeptide sequence also implicitly encompasses conservatively modified variants thereof.

The terms “tumor cell” and “cancer cell” may be used interchangeably herein and refer to individual cells or the total population of cells derived from a tumor or cancer, including both non-tumorigenic cells and cancer stem cells.

The terms “subject” and “patient” are used interchangeably herein to refer to any human or non-human animal in need of treatment. Non-human animals include all vertebrates (e.g., mammals and non-mammals) such as any mammal. Non-limiting examples of mammals include humans, chimpanzees, apes, monkeys, cattle, horses, sheep, goats, swine, rabbits, dogs, cats, rats, mice, and guinea pigs. Non-limiting examples of non-mammals include birds and fish. In some embodiments, the subject is a human.

As used herein, the term “treat,” “treating,” or “treatment” refersreversing, reducing, or arresting the symptoms, clinical signs, and underlying pathology of a condition in manner to improve or stabilize a subject's condition. As used herein, and as well understood in the art “treatment” is an approach for obtaining beneficial or desired results, including clinical results. Beneficial or desired clinical results can include, but are not limited to, alleviation, amelioration, or slowing the progression, of one or more symptoms or conditions associated with a condition, e.g., cancer, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment.

As used herein, the term “therapeutically effective amount” or “effective dose,” refers to an amount of a compound or a conjugate described herein, that elicits the desired biological response in a subject. Such response includes alleviation of the symptoms of the disease or disorder being treated, prevention, inhibition or a delay in the recurrence of symptom of the disease or of the disease itself, an increase in the longevity of the subject compared with the absence of the treatment, or prevention, inhibition or delay in the progression of symptom of the disease or of the disease itself. Determination of the effective amount is well within the capability of those skilled in the art, especially in light of the detailed disclosure provided herein. Toxicity and therapeutic efficacy of compound I can be determined by standard pharmaceutical procedures in cell cultures and in experimental animals. The effective amount of compound or conjugate of the present disclosure or other therapeutic agent to be administered to a subject will depend on the stage, category and status of the multiple myeloma and characteristics of the subject, such as general health, age, sex, body weight and drug tolerance. The effective amount of compound or conjugate of the present disclosure or other therapeutic agent to be administered will also depend on administration route and dosage form. Dosage amount and interval can be adjusted individually to provide plasma levels of the active compound that are sufficient to maintain desired therapeutic effects.

The term “r” or “drug loading” or “drug:antibody ratio” or “drug-to-antibody ratio” or “DAR” refers to the number of photosensitizer moieties per targeting agent (e.g., antibody or antigen-binding fragment), or the number of -L-PS moieties per targeting agent (e.g., antibody or antigen-binding fragment) (T) in conjugate of formula (III). In compositions comprising multiple copies of conjugates of formula (III), “average r” refers to the average number of -L-PS moietiesper targeting agent (e.g., antibody or antigen-binding fragment), also referred to as “average drug loading.”

### Photosensitizer Compound

In a first aspect, the present disclosure provides a photosensitizer compound that is benzoporphyrin analogs. In a 1^{st} embodiment of the first aspect, the photosensitizer compound is represented by the following formula:

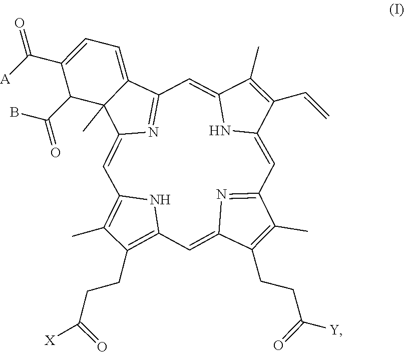

or a pharmaceutically acceptable salt thereof, wherein:
A, B, X and Y are each independently —OH, —OC₁₋₄alkyl, —N(R¹⁰⁰)(R¹⁰¹), or —Z-L^{P}-ker, provided at least one of A, B, X and Y is —N(R¹⁰⁰)(R¹⁰¹), —Z-L^{P}-NH₂, or —Z-L^{P}-ker, and provided at most one of A, B, X and Y is —Z-L^{P}-NH₂ or —Z-L^{P}-ker;
R¹⁰⁰ is H or C₁₋₃alkyl;
R¹⁰¹ is H, C₁₋₃alkyl or —(CH₂—CH₂—O)ₙᵣR¹⁰²;
R¹⁰² is H or Me;
nr is an integer from 1 to 16;
Z is —O— or —NR¹—;
R¹ is H or C₁₋₃alkyl;
L^{P} is a spacer;
ker is a reactive group.

In an alternative 1^{st} embodiment of the first aspect, the photosensitizer compound is represented by the following formula:

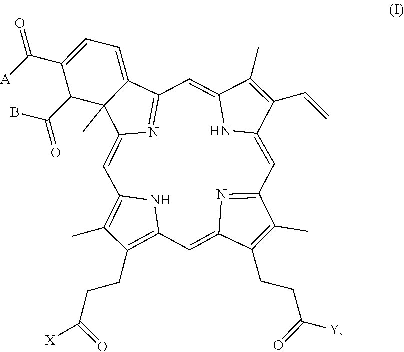

or a pharmaceutically acceptable salt thereof, wherein:
A, B, X and Y are each independently —OH, —OC₁₋₄alkyl, —N(R¹⁰⁰)(R¹⁰¹), or —Z-L^{P}-ker, provided at least one of A, B, X and Y is —N(R¹⁰⁰)(R¹⁰¹) or —Z-L^{P}-ker, and provided at most one of A, B, X and Y is —Z-L^{P}-ker;
R¹⁰⁰ is H or C₁₋₃alkyl;
R¹⁰¹ is H, C₁₋₃alkyl or —(CH₂—CH₂—O)ₙᵣR¹⁰²;
R¹⁰² is H or Me;
nr is an integer from 1 to 16;
Z is —O— or —NR¹—;
R¹ is H or C₁₋₃alkyl;
L^{P} is a spacer;
ker is a reactive group.

In a 2^{nd} embodiment of the first aspect, for the photosensitizer compound of formula (I) or a pharmaceutically acceptable salt thereof, the variables A, B, X and Y are as defined below:
a) A and B are —OCH₃; and one of X and Y is —OCH₃ and the other one of X and Y is —Z-L^{P}-ker;
b) A and B are —OCH₃; and one of X and Y is —OH and the other one of X and Y is —Z-L^{P}-ker;
c) one of A and B is —OCH₃, and the other is —OH; and one of X and Y is —OH and the other one of X and Y is —Z-L^{P}-ker;
d) A and B are —OH; and one of X and Y is —OH and the other one of X and Y is —Z-L^{P}-ker;
e) one of A and B is —OH and the other one of A and B is —OH or —OCH₃; and X and Y are both —NH(CH₂—CH₂—O)ₙᵣR¹⁰², wherein R¹⁰² is H or Me; and nr is an integer from 2 to 8; or
f) one of A and B is —Z-L^{P}-ker and the other one of A and B is —OH or —OCH₃; and X and Y are both —NH(CH₂—CH₂—O)ₙᵣR¹⁰², wherein R¹⁰² is H or Me; and nr is an integer from 2 to 8; and the remaining variables are as defined in the 1^{st} embodiment of the first aspect.

In some embodiments, for the photosensitizer compound of formula (I) or a pharmaceutically acceptable salt thereof, A and B are —OCH₃; and one of X and Y is —OCH₃ and the other one of X and Y is —Z-L^{P}-ker; and the remaining variables are as defined in the 1^{st} embodiment of the first aspect.

In some embodiments, for the photosensitizer compound of formula (I) or a pharmaceutically acceptable salt thereof, A and B are —OCH₃; and one of X and Y is —OH and the other one of X and Y is —Z-L^{P}-ker; and the remaining variables are as defined the 1^{st}embodiment of the first aspect.

In some embodiments, for the photosensitizer compound of formula (I) or a pharmaceutically acceptable salt thereof, one of A and B is —OCH₃, and the other is —OH; and one of X and Y is —OH and the other one of X and Y is —Z-L^{P}-ker; and the remaining variables are as defined in the 1^{st} embodiment of the first aspect.

In some embodiments, for the photosensitizer compound of formula (I) or a pharmaceutically acceptable salt thereof, A and B are —OH; and one of X and Y is —OH and the other one of X and Y is —Z-L^{P}-ker; and the remaining variables are as defined in the 1^{st}embodiment of the first aspect.

In some embodiments, for the photosensitizer compound of formula (I) or a pharmaceutically acceptable salt thereof, one of A and B is —OH and the other one of A and B is —OH or —OCH₃; and X and Y are both —NH(CH₂—CH₂—O)ₙᵣR¹⁰², wherein R¹⁰² is H or Me; and nr is an integer from 2 to 8; and the remaining variables are as defined in the 1^{st} embodiment of the first aspect.

In some embodiments, for the photosensitizer compound of formula (I) or a pharmaceutically acceptable salt thereof, one of A and B is —Z-L^{P}-ker and the other one of A and B is —OH or —OCH₃; and X and Y are both —NH(CH₂—CH₂—O)ₙᵣR¹⁰², wherein R¹⁰² is H or Me; and nr is an integer from 2 to 8; and the remaining variables are as defined in the 1^{st} embodiment of the first aspect.

In a 3^{rd} embodiment of the first aspect, the photosensitizer compound is represented by formula (IA):

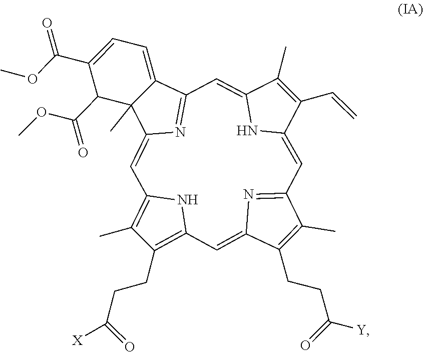

or a pharmaceutically acceptable salt thereof, wherein one of X and Y is —Z-L^{P}-ker and other is —OH; and the remaining variables are as defined in the 1^{st} embodiment of the first aspect.

In a 4^{th} embodiment of the first aspect, the photosensitizer compound is represented by formula (IB):

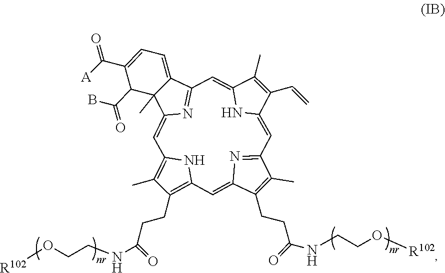

or a pharmaceutically acceptable salt thereof, wherein one of A and B is —OH or —Z-L^{P}-ker and the other is —OH or —OCH₃; R¹⁰² is H or Me; and nr is an integer from 2 to 8; and the remaining variables are as defined in the 1^{st} embodiment of the first aspect.

In a 5^{th} embodiment of the first aspect, for the photosensitizer compound of formula (I), (IA) or (IB), or a pharmaceutically acceptable salt thereof, ker is a reactive group selected from: a maleimide group, a thiol group, a cyclooctyne group, an azido group, a hydrazide group, a tetrazine group, cyclooctene group, a ketone group, and an aldehyde group; and the remaining variables are as described in the 1^{st}, 2^{nd}, 3^{rd} or 4^{th} embodiment of the first aspect. In some embodiment, ker is

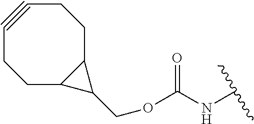 [SMILES: *NC(=O)OCC1C2CCC#CCCC21]

or —N₃; and the remaining variables are as described in the 1^{st}, 2^{nd}, 3^{rd} or 4^{th} embodiment of the first aspect or any embodiments described therein.

In a 6^{th} embodiment of the fist aspect, for the photosensitizer compound of formula (I), (IA) or (IB), or a pharmaceutically acceptable salt thereof, Z is —NH—; and the remaining variables are as described in the 1^{st}, 2^{nd}, 3^{rd} 4^{th} or 5^{th} embodiment of the first aspect or any embodiments described therein.

In a 7^{th} embodiment of the fist aspect, for the photosensitizer compound of formula (I), (IA) or (IB), or a pharmaceutically acceptable salt thereof, Z is —O—; and the remaining variables are as described in the 1^{st}, 2^{nd}, 3^{rd} 4^{th} or 5^{th} embodiment of the first aspect or any embodiments described therein.

In an 8^{th} embodiment of the fist aspect, for the photosensitizer compound of formula (I), (IA) or (IB), or a pharmaceutically acceptable salt thereof, L^{P} is a non-cleavable spacer; and the remaining variables are as described in the 1^{st}, 2^{nd}, 3^{rd}, 4^{th}, 5^{th}, 6^{th} or 7^{th} embodiment of the first aspect or any embodiments described therein. In some embodiments, L^{P} comprises a hydrophilic moiety. In some embodiments, L^{P} comprises a PEG moiety.

In a 9^{th} embodiment of the fist aspect, for the photosensitizer compound of formula (I), (IA) or (IB), or a pharmaceutically acceptable salt thereof, L^{P} is *—CH₂CH₂—(OCH₂CH₂)ₘ—, wherein * is the site connected to ker; m is an integer from 0 to 30; and the remaining variables are as described in the 1^{st}, 2^{nd}, 3^{rd}, 4^{th}, 5^{th}, 6^{th} or 7^{th} embodiment of the first aspect or any embodiments described therein. In some embodiments, m is an integer from 2 to 16. In some embodiments, m is an integer from 2 to 10. In some embodiments, m is an integer from 2 to 8. In some embodiments, m is an integer from 3 to 5. In some embodiments, m is 2, 3, 4, 5, 6, 7 or 8.

In a 10^{th} embodiment of the fist aspect, for the photosensitizer compound of formula (I), (IA) or (IB), or a pharmaceutically acceptable salt thereof, L^{P} is *—CH₂CH₂—(OCH₂CH₂)₄—; and the remaining variables are as described in the 1^{st}, 2^{nd}, 3^{rd}, 4^{th}, 5^{th}, 6^{th} or 7^{th} embodiment of the first aspect or any embodiments described therein.

In a 11^{th} embodiment of the first aspect, for the photosensitizer compound of formula (I), (IA) or (IB), or a pharmaceutically acceptable salt thereof, L^{P} is a cleavable spacer; and the remaining variables are as described in the 1^{st}, 2^{nd}, 3^{rd} 4^{th}, 5^{th}, 6^{th} or 7^{th} embodiment of the first aspect or any embodiments described therein. In some embodiments, the cleavable space comprises an enzyme labile group, a pH labile group, a disulfide group, a ROS cleavable group, a photocleavable group. In some embodiments, the enzyme labile group is a group cleavable by an esterase, a peptidase, an aminopeptidase, a β-galactosidase, a β-glucuronidase, a carboxylesterase, a caspase, a diaphorase, a histone deacetylase, a legumain, or matrix metalloproteinase. In some embodiments, the enzyme labile group is a group cleavable by cathepsin B.

In a 12^{th} embodiment of the first aspect, for the photosensitizer compound of formula (I), (IA) or (IB), or a pharmaceutically acceptable salt thereof, the spacer represented by L^{P} comprises a peptide and a self-immolative group; and remaining variables are as described in the 1^{st}, 2^{nd}, 3^{rd}, 4^{th}5^{th}, 6^{th} or 7^{th} embodiment of the first aspect or any embodiments described therein.

In a 13^{th} embodiment of the first aspect, for the photosensitizer compound of formula (I), (IA) or (IB), or a pharmaceutically acceptable salt thereof, the spacer L^{P} is presented by the following formula:

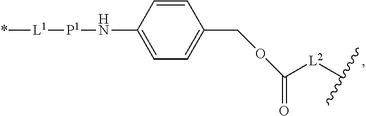

wherein:
*- represents a bond to the reactive group represented by ker;

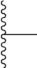

represents a bond to the group Z;
L¹ and L² are each independently a connecting spacer;
P¹ is a peptide comprising 2 to 5 amino acid residues; and the remaining variables are as described in the 1^{st}, 2^{nd}, 3^{rd} 4^{th}, 5^{th} or 7^{th} embodiment of the first aspect or any embodiments described therein.

In a 14^{th} embodiment of the first aspect, for the photosensitizer compound of formula (I), (IA) or (IB), or a pharmaceutically acceptable salt thereof, P¹ is a peptide selected from Phe-Arg-Arg-Gly, Glu-Val-Cit, Val-Cit, Cit-Val, Gly-Gly-Phe, Val-Ala, or Ala-Val; and the remaining variables are as described in the 13^{th} embodiment of the first aspect or any embodiments described therein.

In a 15^{th} embodiment of the first aspect, for the photosensitizer compound of formula (I), (IA) or (IB), or a pharmaceutically acceptable salt thereof, P¹ is:

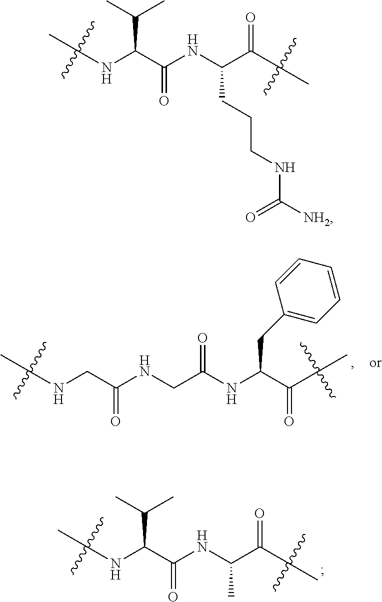

and the remaining variables are as described in the 13^{th} embodiment of the first aspect or any embodiments described therein.

In a 16^{th} embodiment of the first aspect, for the photosensitizer compound of formula (I), (IA) or (IB), or a pharmaceutically acceptable salt thereof, L¹ comprises a hydrophilic moiety; and the remaining variables are as described in the 13^{th}, 14^{th} or 15^{th} embodiment or any embodiments described therein. In some embodiment, L¹ comprises a PEG moiety.

In a 17^{th} embodiment of the first aspect, for the photosensitizer compound of formula (I), (IA) or (IB), or a pharmaceutically acceptable salt thereof, L¹ is *—CH₂—CH₂—(OCH₂CH₂)ₙ—C(═O)—**; n is an integer from 0 to 30; *- represents a bond to the reactive group represented by ker; and **- represents a bond to P¹; and the remaining variables are as described in the 13^{th}, 14^{th} or 15^{th} embodiment or any embodiments described therein. In some embodiments, n is an integer from 2 to 16. In some embodiments, n is an integer from 2 to 10. In some embodiment, n is an integer from 2 to 8. In some embodiments, n is an integer from 3 to 5. In some embodiments, n is 2, 3, 4, 5, 6, 7 or 8.

In a 18^{th} embodiment of the first aspect, for the photosensitizer compound of formula (I), (IA) or (IB), or a pharmaceutically acceptable salt thereof, L¹ is *—CH₂—CH₂—(OCH₂CH₂)₃—C(═O)—**; the remaining variables are as described in the 13^{th}, 14^{th} or 15^{th} embodiment or any embodiments described therein.

In a 19^{th} embodiment of the first aspect, for the photosensitizer compound of formula (I), (IA) or (IB), or a pharmaceutically acceptable salt thereof, L² is —NH—C₁₋₆alkyl-**, wherein ** is the site connected to Z; and the remaining variables are as described in the 13^{th}, 14^{th}, 15^{th}, 16^{th}, 17^{th} or 18^{th} embodiment or any embodiments described therein.

In a 20^{th} embodiment of the first aspect, for the photosensitizer compound of formula (I), (IA) or (IB), or a pharmaceutically acceptable salt thereof, L² is —NH—CH₂CH₂—**, wherein ** is the site connected to Z; and the remaining variables are as described in the 13^{th}14^{th}, 15^{th}, 16^{th}, 17^{th} or 18^{th} embodiment or any embodiments described therein.

In a 21^{st} embodiment of the first aspect, the photosensitizer compound of the present disclosure is selected from:

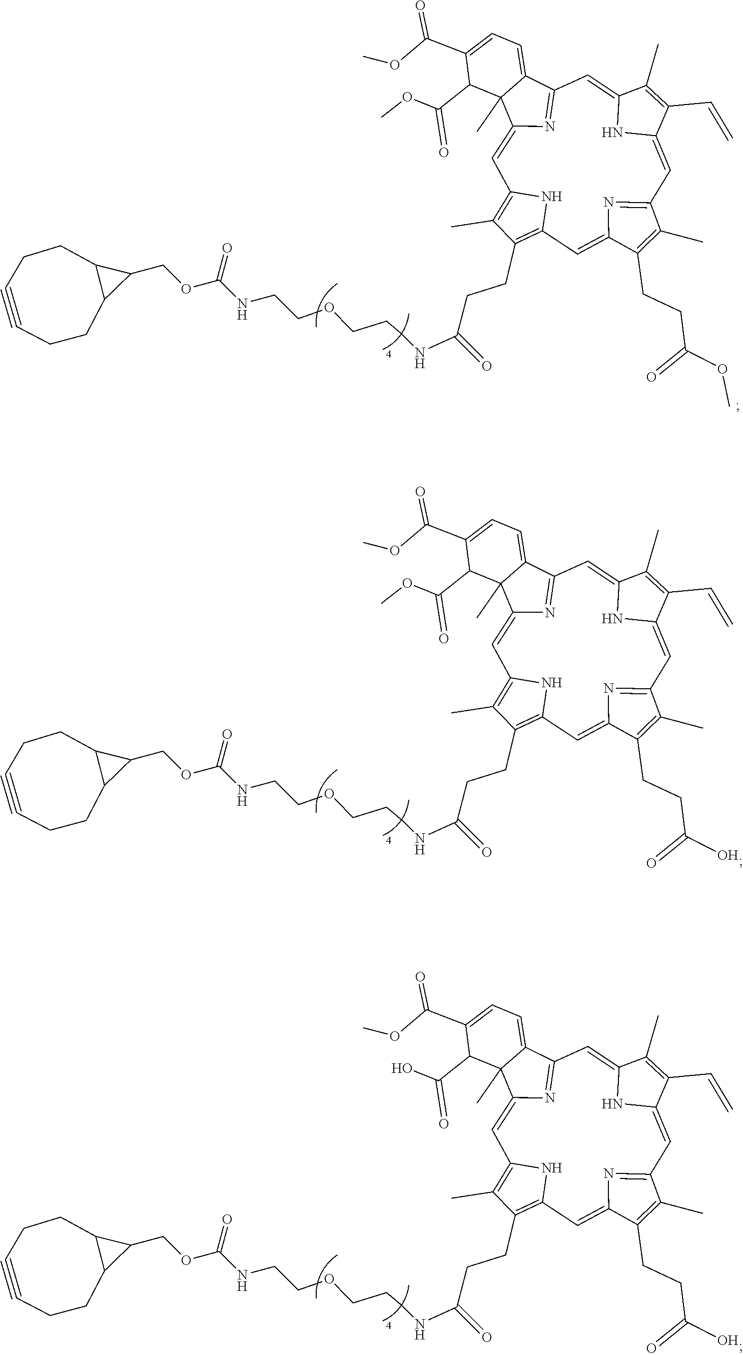

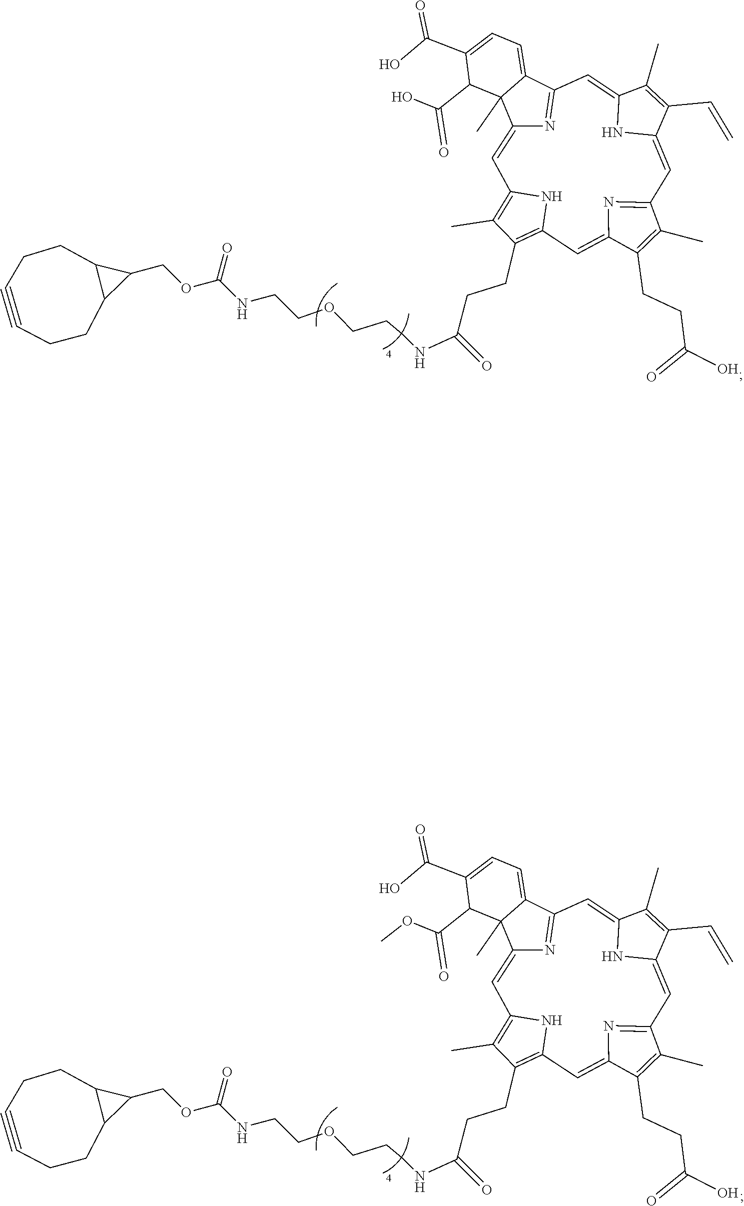

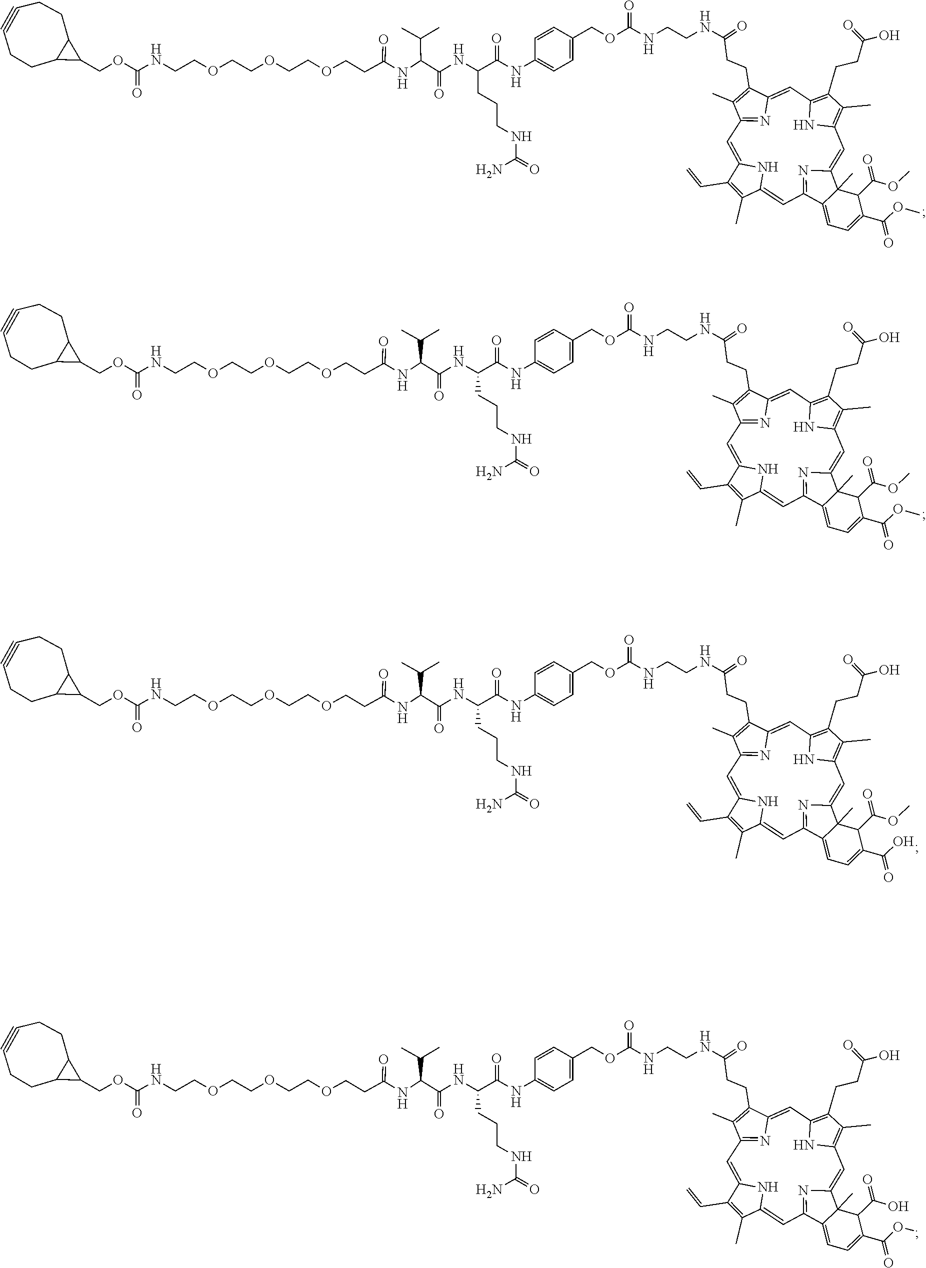

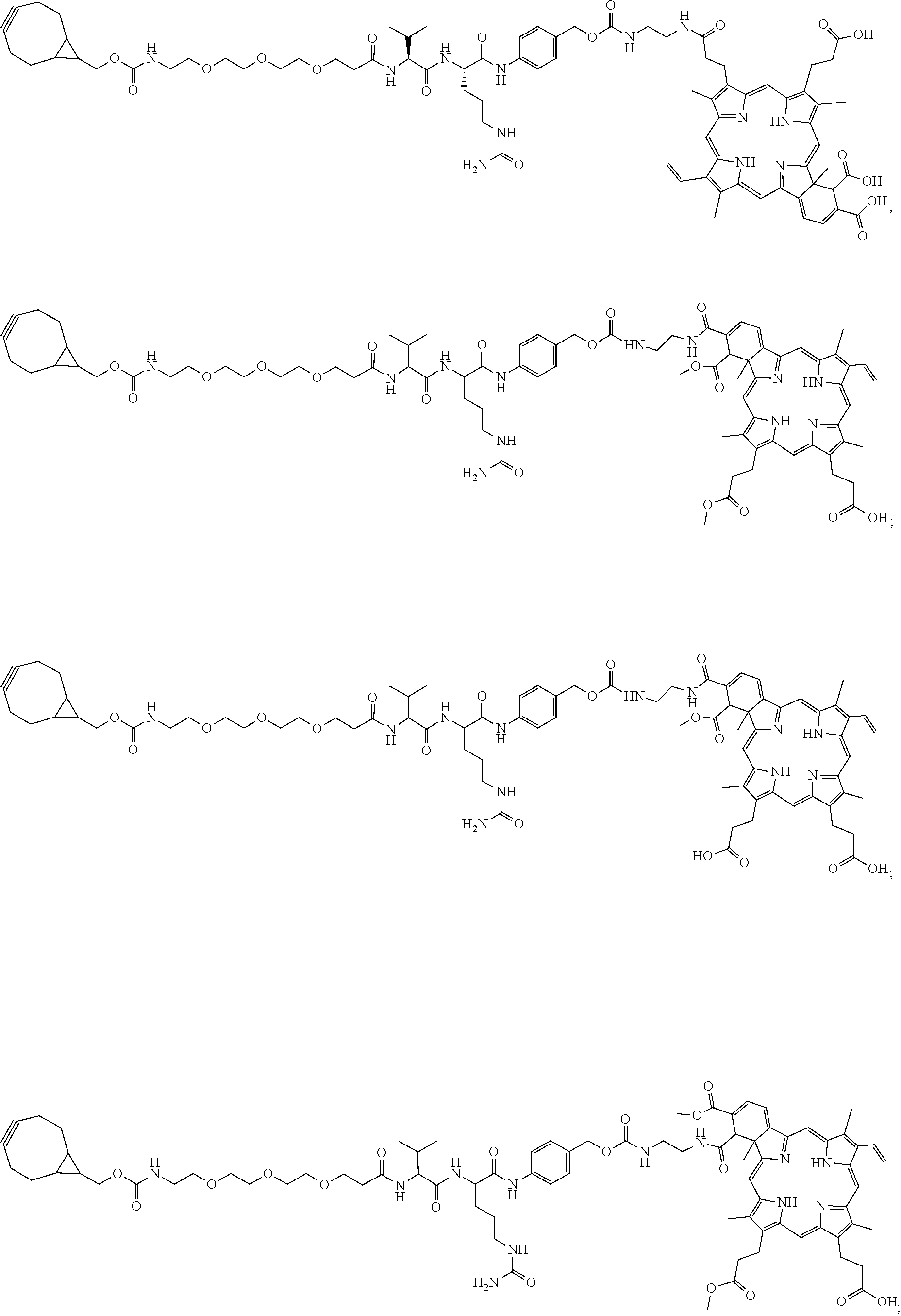

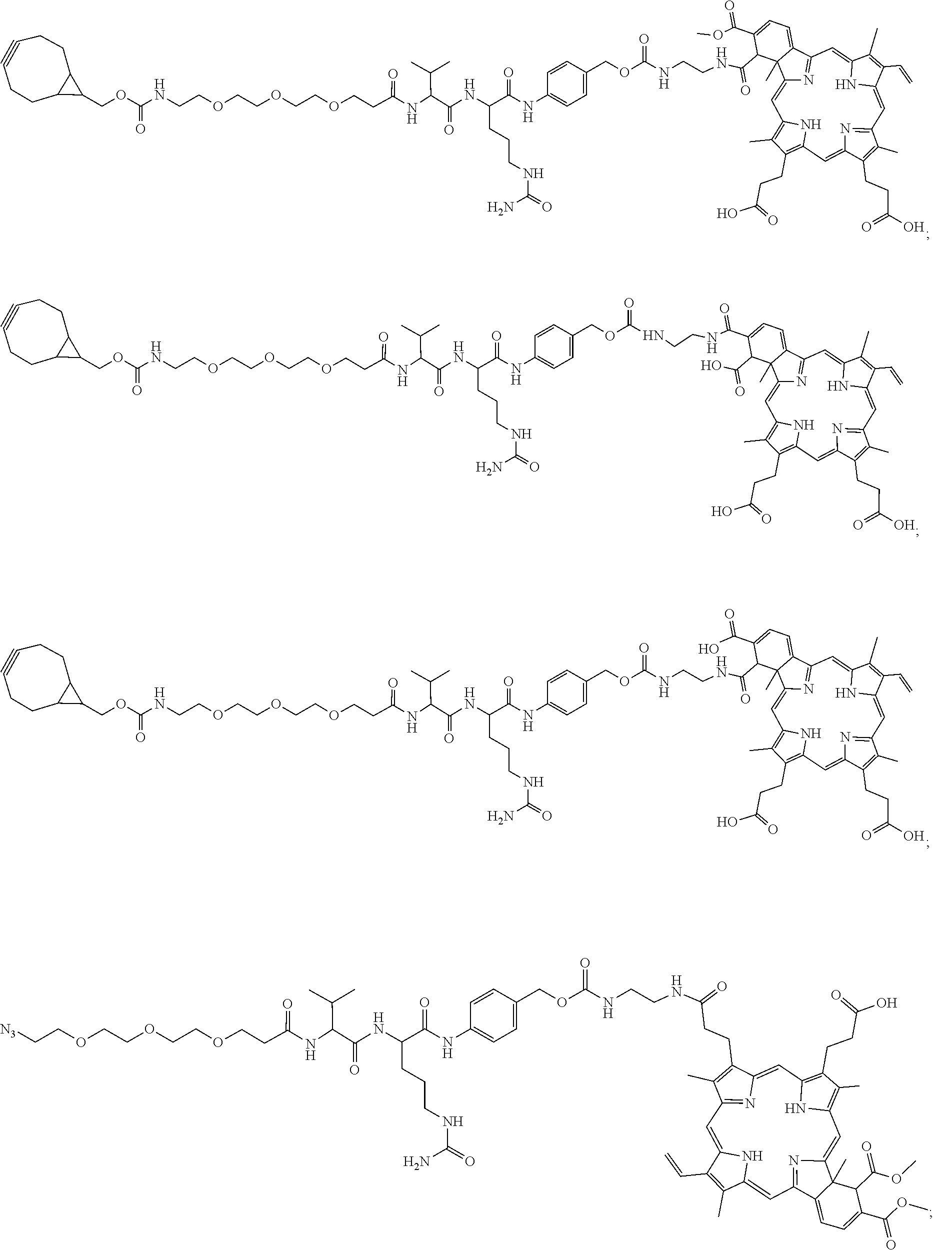

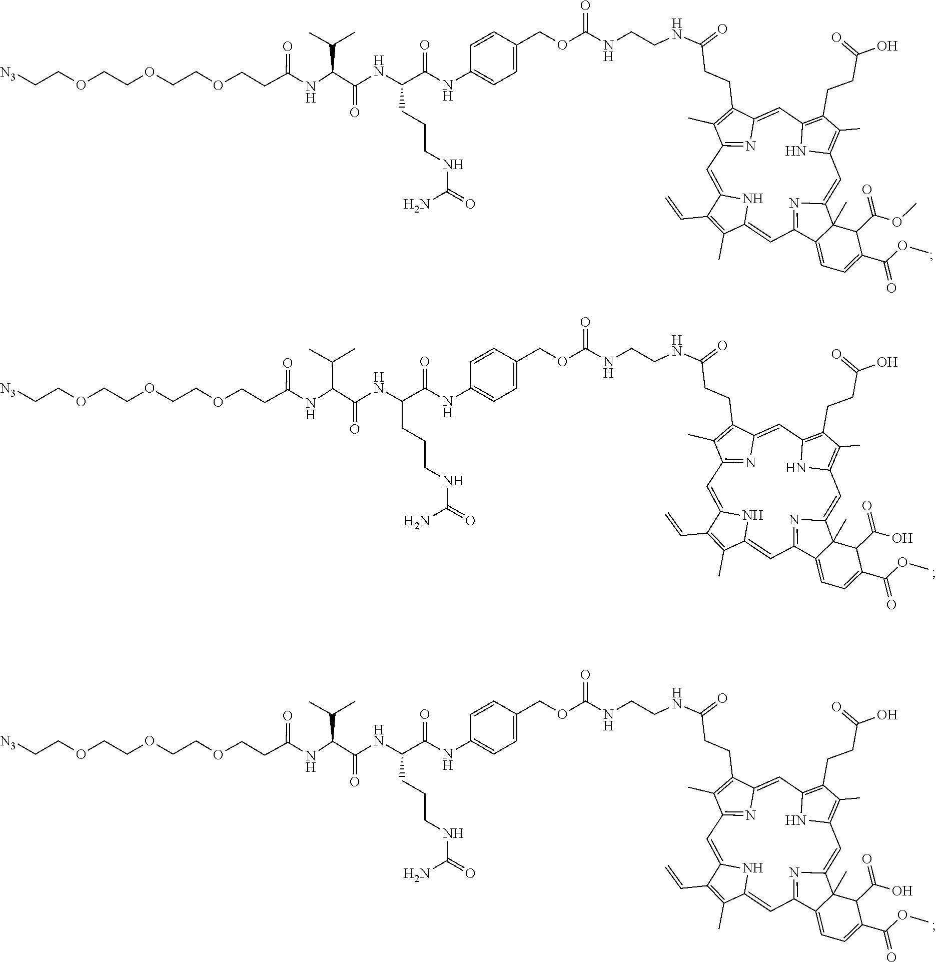

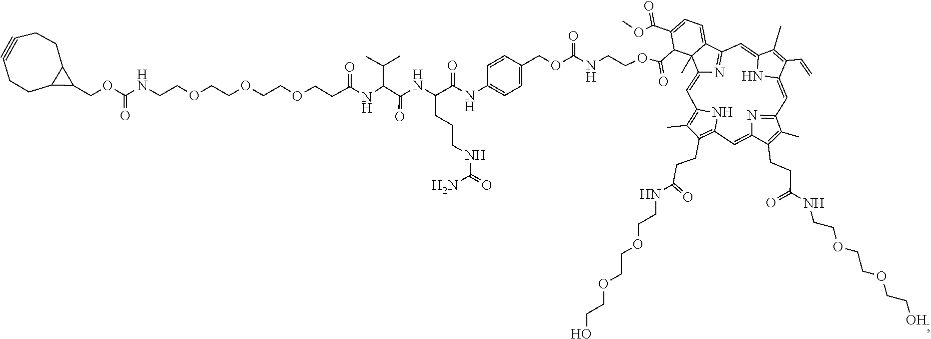 [SMILES: C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)OC)C1C(=O)OCCNC(=O)OCc1ccc(NC(=O)C(CCCNC(N)=O)NC(=O)C(NC(=O)CCOCCOCCOCCNC(=O)OCC2C3CCC#CCCC32)C(C)C)cc1]

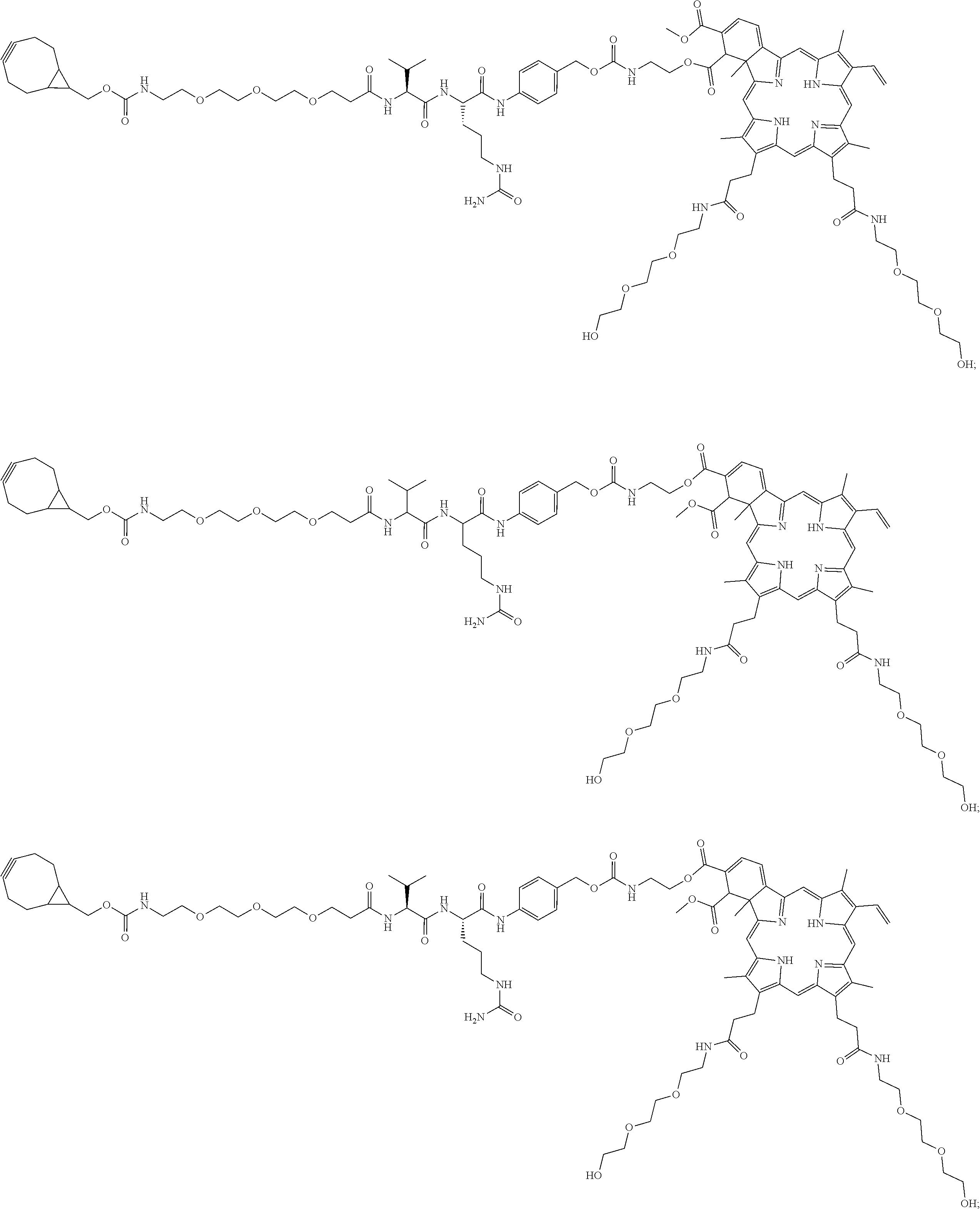

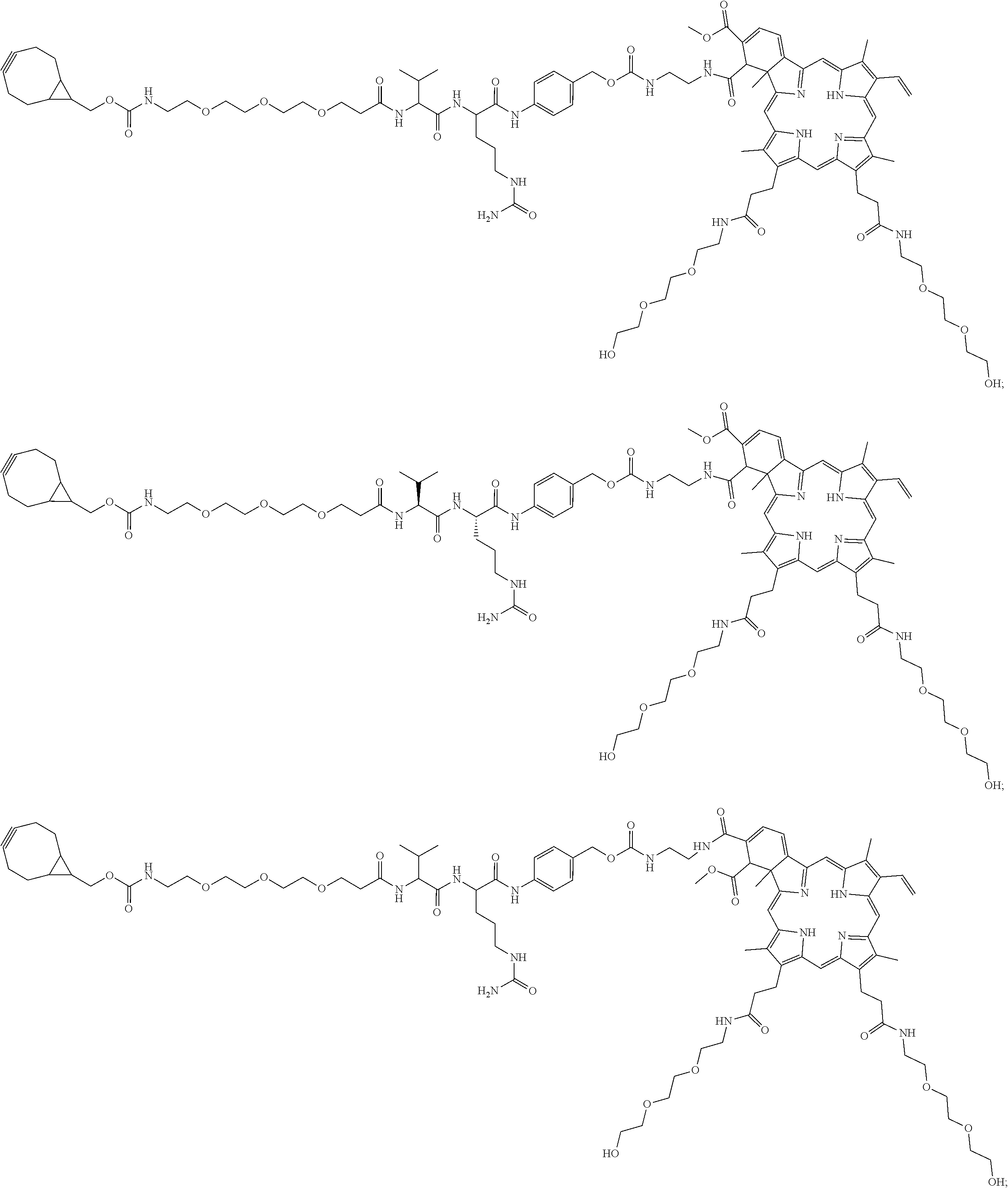

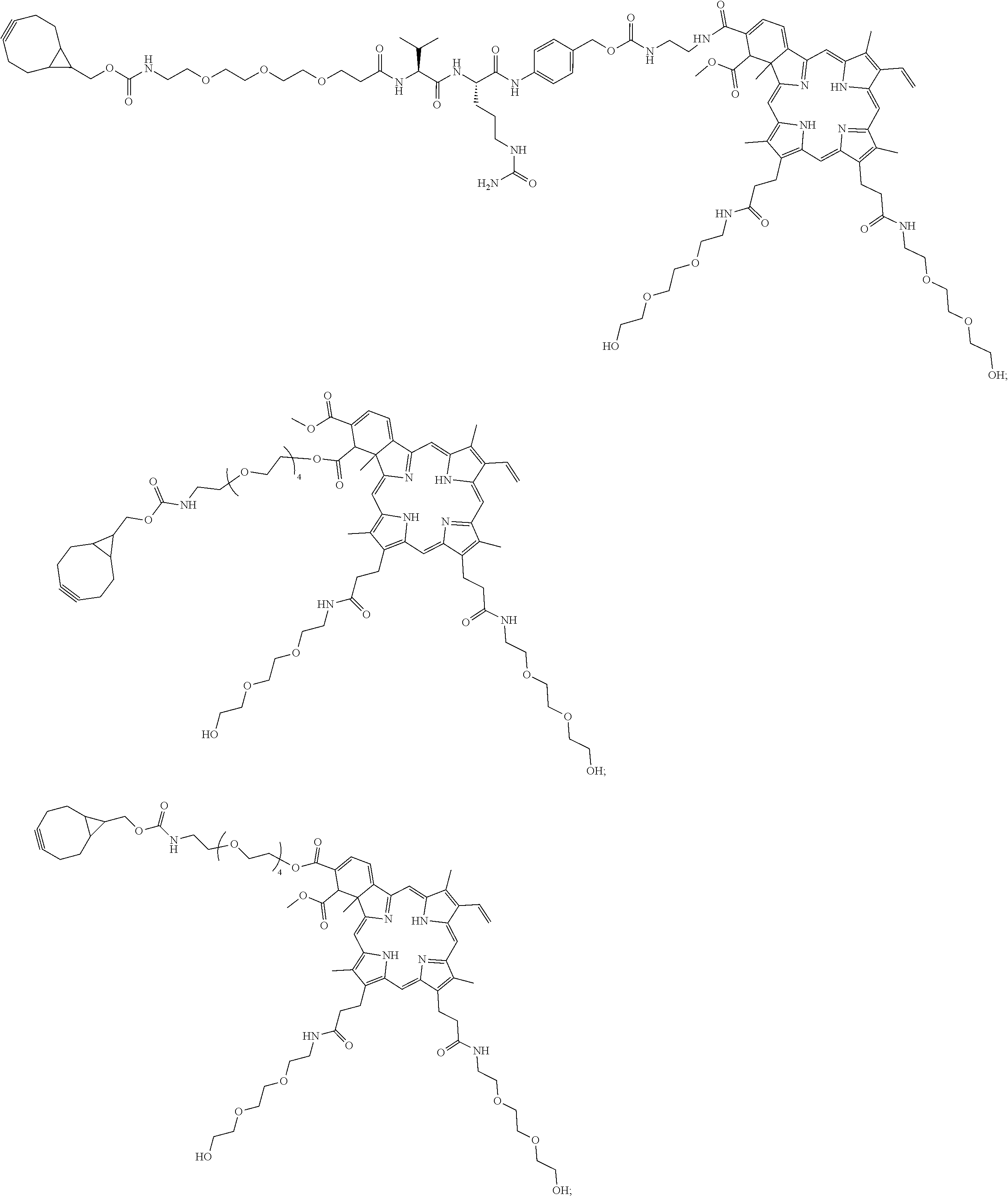

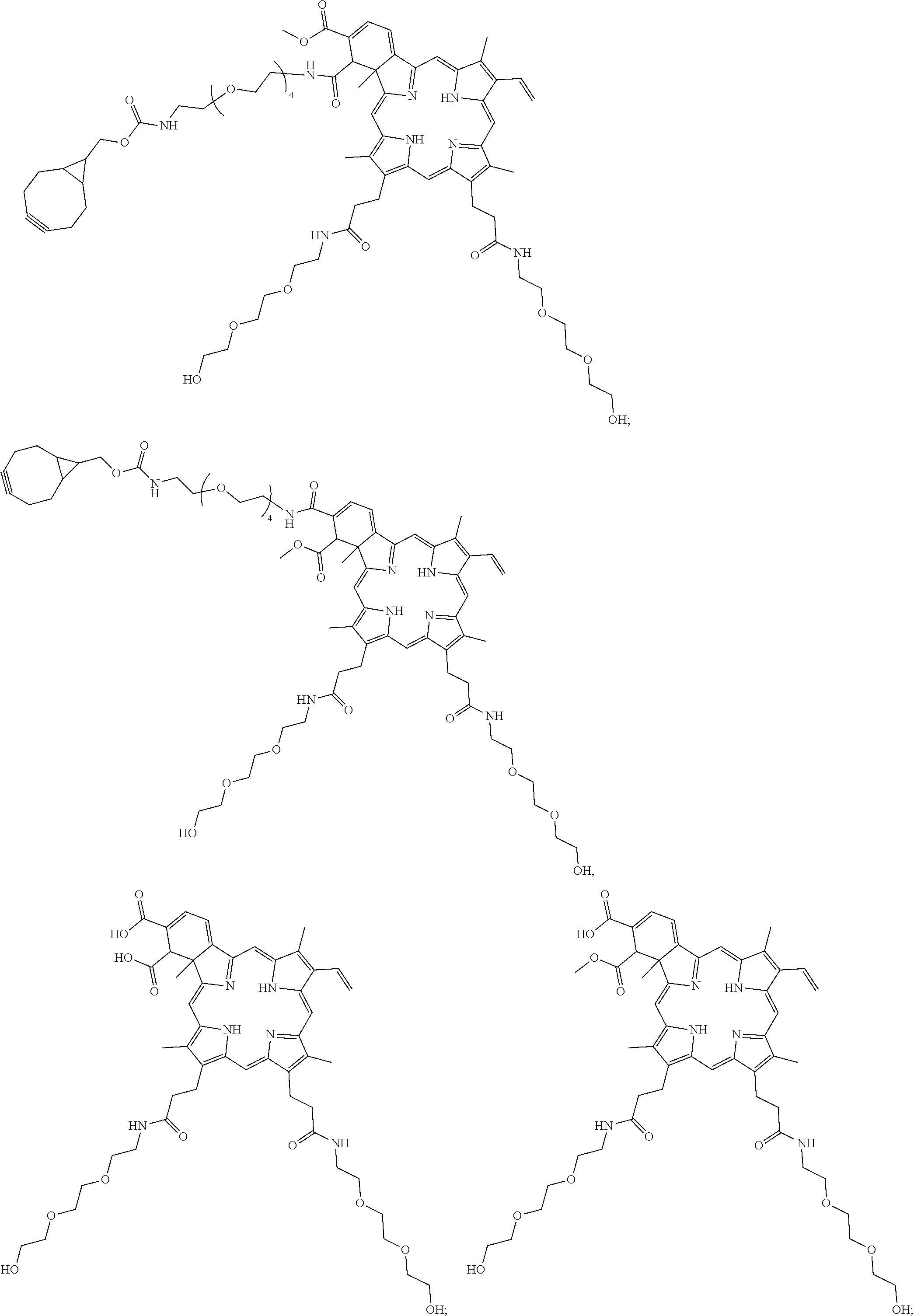

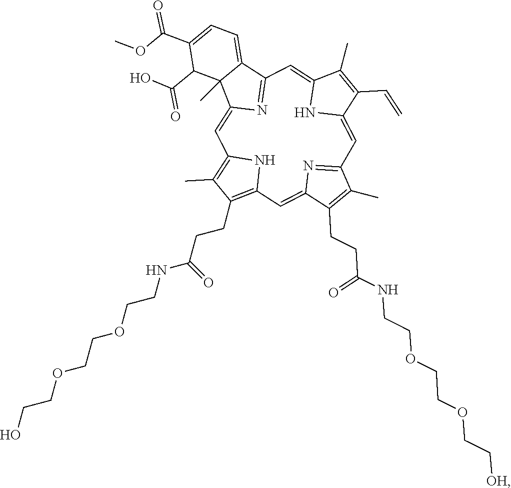 [SMILES: C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)OC)C1C(=O)O]

or a pharmaceutically acceptable salt thereof, a regioisomer, or a pharmaceutically acceptable salt of the regioisomer. Alternatively, as part of the 21^{st} embodiment of the first aspect, the photosensitizer compound of the present disclosure is selected from:

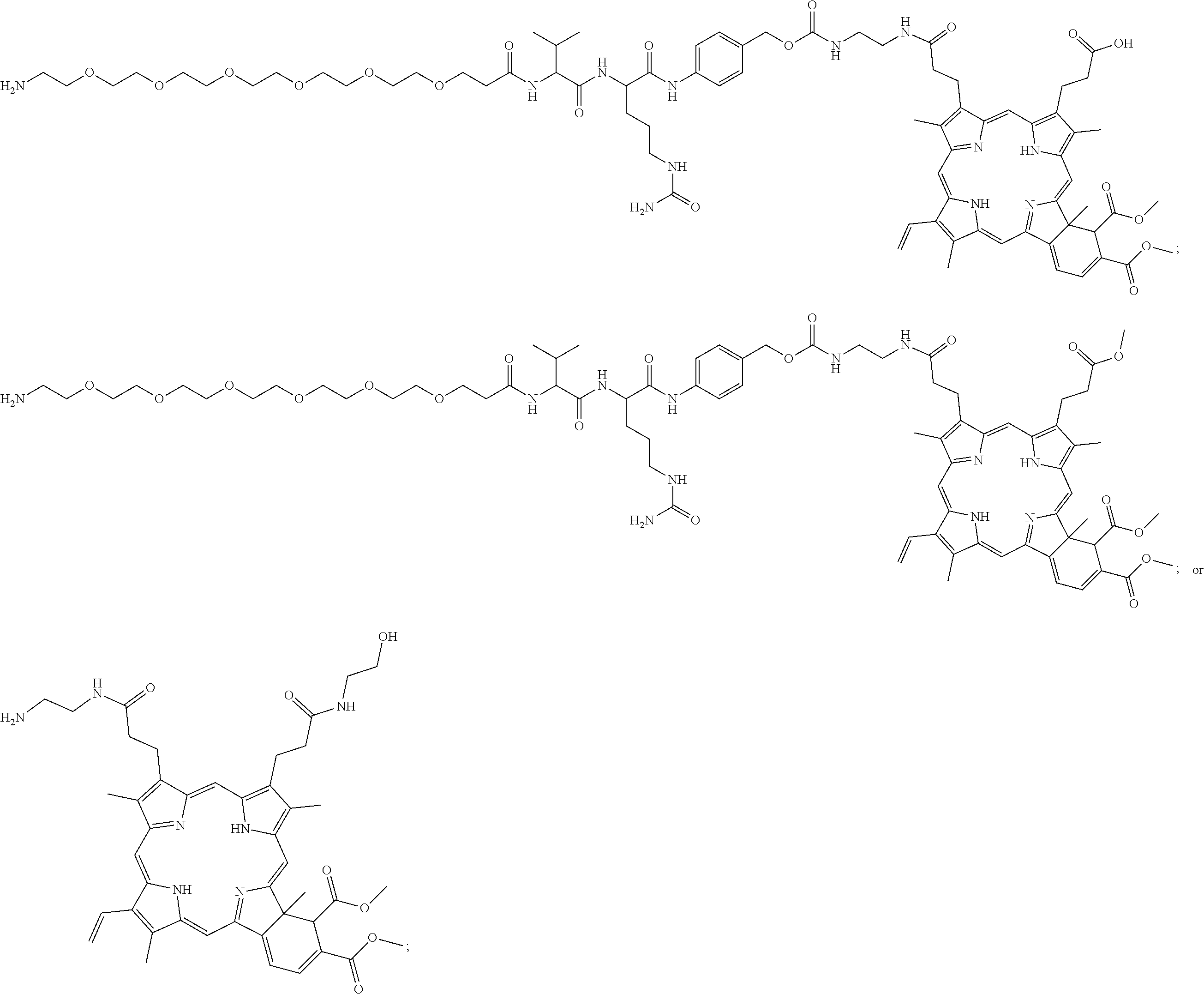 [SMILES: C=Cc1c(C)c2cc3nc(cc4[nH]c(cc5nc(cc1[nH]2)C(C)=C5CCC(=O)NCCNC(=O)OCc1ccc(NC(=O)C(CCCNC(N)=O)NC(=O)C(NC(=O)CCOCCOCCOCCOCCOCCOCCN)C(C)C)cc1)c(CCC(=O)O)c4C)C1(C)C3=CC=C(C(=O)OC)C1C(=O)OC ; C=Cc1c(C)c2cc3nc(cc4[nH]c(cc5nc(cc1[nH]2)C(C)=C5CCC(=O)NCCNC(=O)OCc1ccc(NC(=O)C(CCCNC(N)=O)NC(=O)C(NC(=O)CCOCCOCCOCCOCCOCCOCCN)C(C)C)cc1)c(CCC(=O)OC)c4C)C1(C)C3=CC=C(C(=O)OC)C1C(=O)OC ; C=Cc1c(C)c2cc3nc(cc4[nH]c(cc5nc(cc1[nH]2)C(C)=C5CCC(=O)NCCN)c(CCC(=O)NCCO)c4C)C1(C)C3=CC=C(C(=O)OC)C1C(=O)OC]

or a pharmaceutically acceptable salt thereof, a regioisomer, or a pharmaceutically acceptable salt of the regioisomer.

### Linker-Photosensitizer Compound

In a second aspect, the present disclosure provides a linker-photosensitizer compound, wherein the photosensitizer compound of the first aspect is modified with a linker having a reactive group that can form a covalent bond with the targeting agent (e.g., antibodies or antigen-binding fragment thereof) described below. In a 1^{st} embodiment of the second aspect, the linker-photosensitizer compound is represented by formula (II):

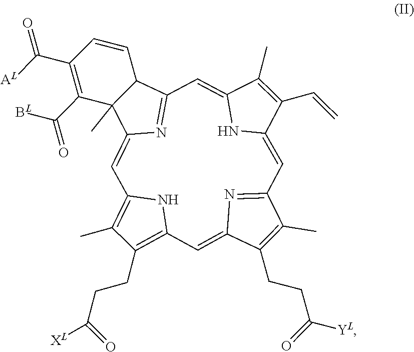

or a pharmaceutically acceptable salt thereof, wherein:
A^{L}, B^{L}, X^{L} and Y^{L} are each independently —OH, —OC₁₋₄alkyl, —N(R¹⁰⁰)(R¹⁰¹) or —Z-L^{P}-linker-L^{A}-R^{A}, provided one of A^{L}, B^{L}, X^{L} and Y^{L} is —Z-L^{P}-linker-L^{A}-R^{A};
R¹⁰⁰ is H or C₁₋₃alkyl;
R¹⁰¹ is H, C₁₋₃alkyl or —(CH₂—CH₂—O)ₙᵣR¹⁰²;
R¹⁰² is H or Me;
nr is an integer from 1 to 16;
Z is —O— or —NR¹—;
R¹ is H or C₁₋₃alkyl;
L^{P} and L^{A} are each independently a spacer;
Linker is connection moiety formed by a first reactive group attached to L^{P} and a second reactive group attached to L^{A}; and
R^{A} is a reactive group that can form a covalent bond with a targeting agent.

In a 2^{nd} embodiment of the second aspect, for the linker-photosensitizer compound of formula (II) or a pharmaceutically acceptable salt thereof, R^{A} is a reactive ester, —NH₂, or a maleimide group; and the remaining variables are as defined in the 1^{st} embodiment of the second aspect.

In a 3^{rd} embodiment of the second aspect, for the linker-photosensitizer compound of formula (II) or a pharmaceutically acceptable salt thereof, R^{A} is

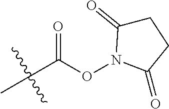 [SMILES: *C(=O)ON1C(=O)CCC1=O]

or —NH₂; and the remaining variables are as defined in the 1^{st} embodiment of the second aspect.

In a 4^{th} embodiment of the second aspect, for the linker-photosensitizer compound of formula (II) or a pharmaceutically acceptable salt thereof, L^{A} is a spacer comprising a hydrophilic moiety; and the remaining variables are as defined in the 1^{st}, 2^{nd} or 3^{rd}embodiment of the second aspect. In some embodiments, L^{A} is a spacer comprising a PEG moiety.

In a 5^{th} embodiment of the second aspect, for the linker-photosensitizer compound of formula (II) or a pharmaceutically acceptable salt thereof, L^{A} is *—CH₂CH₂(OCH₂CH₂)ₚ—, wherein * is the site connected to R^{A}; and p is an integer from 0 to 30; and the remaining variables are as defined in the 1^{st}, 2^{nd} or 3^{rd} embodiment of the second aspect or any embodiments described therein. In some embodiments, p is an integer from 2 to 16. In some embodiments, p is an integer from 2 to 10. In some embodiment, p is an integer from 2 to 8. In some embodiments, p is an integer from 3 to 5. In some embodiments, p is 2, 3, 4, 5, 6, 7 or 8. In some embodiments, p is 6.

In a 6^{th} embodiment of the second aspect, for the linker-photosensitizer compound of formula (II) or a pharmaceutically acceptable salt thereof, the first reactive group and the second reactive group are each selected from a maleimide group, a thiol group, a cyclooctyne group, and an azido group; and the remaining variables are as defined in the 1^{st}, 2^{nd}, 3^{rd}, 4^{th} or 5^{th} embodiment of the second aspect or any embodiments described therein.

In a 7^{th} embodiment of the second aspect, for the linker-photosensitizer compound of formula (II) or a pharmaceutically acceptable salt thereof, Linker in formula (II) is is represented by the following formula:

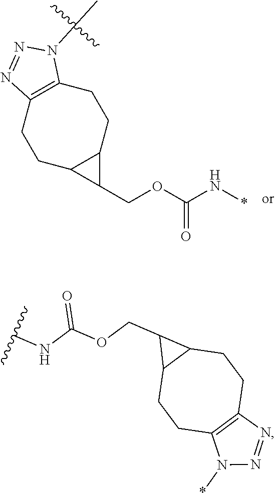 [SMILES: *NC(=O)OCC1C2CCc3nnn(*)c3CCC21 ; *NC(=O)OCC1C2CCc3nnn(*)c3CCC21]

wherein *- represents a bond to L^{P} and

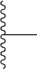

represents a bond to L^{A}; and the remaining variables are as defined in the 1^{st}, 2^{nd}, 3^{rd}, 4^{th} or 5^{th} embodiment of the second aspect or any embodiments described therein.

In a 8^{th} embodiment of the second aspect, for the linker-photosensitizer compound of formula (II) or a pharmaceutically acceptable salt thereof, A^{L}, B^{L}, X^{L} and Y^{L} are defined as below:
a) A^{L} and B^{L} are —OCH₃; and one of X^{L} and Y^{L} is —OCH₃ and the other one of X^{L} and Y^{L} is —Z-L^{P}-linker-L^{A}-R^{A}.
b) A^{L} and B^{L} are —OCH₃; and one of X^{L} and Y^{L} is —OH and the other one of X^{L} and Y^{L} is —Z-L^{P}-linker-L^{A}-R^{A}.
c) one of A^{L} and B^{L} is —OCH₃, and the other one of A^{L} and B^{L} is —OH; and one of X^{L} and Y^{L} is —OH and the other one of X^{L} and Y^{L} is —Z-L^{P}-linker-L^{A}-R^{A};
d) A^{L} and B^{L} are —OH; and one of X^{L} and Y^{L} is —OH and the other one of X^{L} and Y^{L} is —Z-L^{P}-linker-L^{A}-R^{A}; or
e) one of A^{L} and B^{L} is —Z-L^{P}-linker-L^{A}-R^{A} and the other one of A^{L} and B^{L} is —OH or —OCH₃; and X^{L} and Y^{L} are both —NH(CH₂—CH₂—O)ₙᵣR¹⁰², wherein R¹⁰² is H or Me; and nr is an integer from 2 to 8;
and the remaining variables are as defined in the 1^{st}, 2^{nd}, 3^{rd}, 4^{th}, 5^{th}, 6^{th} or 7^{th} embodiment of the second aspect or any embodiments described therein.

In some embodiments, for the linker-photosensitizer compound of formula (II) or a pharmaceutically acceptable salt thereof, A^{L} and B^{L} are —OCH₃; and one of X^{L} and Y^{L} is —OCH₃ and the other one of X^{L} and Y^{L} is —Z-L^{P}-linker-L^{A}-R^{A}.

In some embodiments, for the linker-photosensitizer compound of formula (II) or a pharmaceutically acceptable salt thereof, A^{L} and B^{L} are —OCH₃; and one of X^{L} and Y^{L} is —OH and the other one of X^{L} and Y^{L} is —Z-L^{P}-linker-L^{A}-R^{A}.

In some embodiments, for the linker-photosensitizer compound of formula (II) or a pharmaceutically acceptable salt thereof, one of A^{L} and B^{L} is —OCH₃, and the other one of A^{L} and B^{L} is —OH; and one of X^{L} and Y^{L} is —OH and the other one of X^{L} and Y^{L} is —Z-L^{P}-linker-L^{A}-R^{A}.

In some embodiments, for the linker-photosensitizer compound of formula (II) or a pharmaceutically acceptable salt thereof, A^{L} and B^{L} are —OH; and one of X^{L} and Y^{L} is —OH and the other one of X^{L} and Y^{L} is —Z-L^{P}-linker-L^{A}-R^{A}.

In some embodiments, for the linker-photosensitizer compound of formula (II) or a pharmaceutically acceptable salt thereof, one of A^{L} and B^{L} is —Z-L^{P}-linker-L^{A}-R^{A} and the other one of A^{L} and B^{L} is —OH or —OCH₃; and X^{L} and Y^{L} are both —NH(CH₂—CH₂—O)ₙᵣR¹⁰² wherein R¹⁰² is H or Me; and nr is an integer from 2 to 8.

In a 9^{th} embodiment of the second aspect, the linker-photosensitizer compound of the present disclosure is represented by formula (IIA):

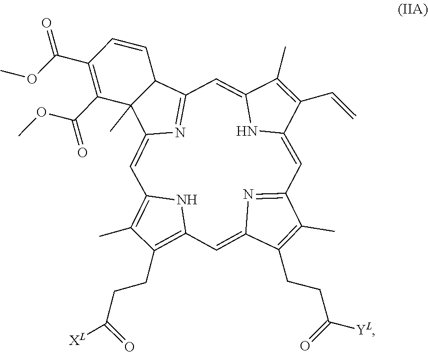

or a pharmaceutically acceptable salt thereof, wherein one of X^{L} and Y^{L} is —Z-L^{P}-linker-L^{A}-R^{A} and the other is —OH; and the remaining variables are as defined in the 1^{st}, 2^{nd}, 3^{rd}, 4^{th}, 5^{th}, 6^{th} or 7^{th} embodiment of the second aspect or any embodiments described therein.

In a 10^{th} embodiment of the second aspect, the linker-photosensitizer compound of the present disclosure is represented by formula (IIB):

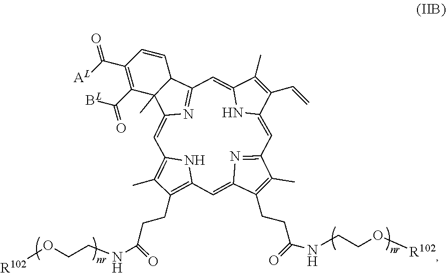

or a pharmaceutically acceptable salt thereof, wherein one of A^{L} and B^{L} is —Z-L^{P}-linker-L^{A}-R^{A} and the other is —OH or —OCH₃; R¹⁰² is H or Me; nr is an integer from 2 to 8; and the remaining variables are as defined in the 1^{st}, 2^{nd}, 3^{rd}, 4^{th}, 5^{th}, 6^{th} or 7^{th} embodiment of the second aspect or any embodiments described therein.

In a 11^{th} embodiment of the second aspect, for the linker-photosensitizer compound of formula (II), (IIA), (IIB) or a pharmaceutically acceptable salt thereof, Z is —NH—; and the remaining variables are as defined in the 1^{st}, 2^{nd}, 3^{rd}, 4^{th}, 5^{th}, 6^{th}, 7^{th}, 8^{th}, 9^{th} or 10^{th} embodiment of the second aspect or any embodiments described therein.

In a 12^{th} embodiment of the second aspect, for the linker-photosensitizer compound of formula (II), (IIA), (IIB) or a pharmaceutically acceptable salt thereof, Z is —O—; and the remaining variables are as defined in the 1^{st}, 2^{nd}, 3^{rd}, 4^{th}, 5^{th}, 6^{th}, 7^{th}, 8^{th}, 9^{th} or 10^{th} embodiment of the second aspect or any embodiments described therein.

In a 13^{th} embodiment of the second aspect, for the linker-photosensitizer compound of formula (II), (IIA), (IIB) or a pharmaceutically acceptable salt thereof, L^{P} is a non-cleavable spacer; and the remaining variables are as defined in the 1^{st}, 2^{nd}, 3^{rd}, 4^{th}, 5^{th}, 6^{th}, 7th, 8^{th}, 9^{th}, 10^{th}, 11^{th} or 12^{th} embodiment of the second aspect or any embodiments described therein. In some embodiments, L^{P} comprises a hydrophilic moiety. In some embodiments, L^{P} comprises a PEG moiety.

In a 14^{th} embodiment of the second aspect, for the linker-photosensitizer compound of formula (II), (IIA), (IIB) or a pharmaceutically acceptable salt thereof, L^{P} is *—CH₂CH₂—(OCH₂CH₂)ₘ—, wherein * is the site connected to the Linker, and m is an integer from 0 to 30; and the remaining variables are as defined in the 1^{st}, 2^{nd}, 3^{rd}, 4^{th}, 5^{th}, 6^{th}, 7^{th}, 8^{th}, 9^{th}, 10^{th}, 11^{th} or 12^{th} embodiment of the second aspect or any embodiments described therein. In some embodiments, m is an integer from 2 to 16. In some embodiments, m is an integer from 2 to 10. In some embodiments, m is an integer from 2 to 8. In some embodiments, m is an integer from 3 to 5. In some embodiments, m is 2, 3, 4, 5, 6, 7 or 8.

In a 15^{th} embodiment of the second aspect, for the linker-photosensitizer compound of formula (II), (IIA), (IIB) or a pharmaceutically acceptable salt thereof, L^{P} is *—CH₂CH₂—(OCH₂CH₂)₄—, wherein * is the site connected to the Linker, and the remaining variables are as defined in the 14^{th} embodiment of the second aspect or any embodiments described therein.

In a 16^{th} embodiment of the second aspect, for the linker-photosensitizer compound of formula (II), (IIA), (IIB) or a pharmaceutically acceptable salt thereof, L^{P} is a cleavable spacer; and the remaining variables are as defined in the 1^{st}, 2^{nd}, 3^{rd}, 4^{th}, 5^{th}, 6^{th}, 7th, 8^{th}, 9^{th}, 10^{th}, 11^{th} or 12^{th} embodiment of the second aspect or any embodiments described therein. In some embodiments, the cleavable spacer comprises an enzyme labile group, a pH labile group, a disulfide group, a ROS cleavable group, a photocleavable group. In some embodiments, the enzyme labile group is a group cleavable by an esterase, a peptidase, an aminopeptidase, a β-galactosidase, a β-glucuronidase, a carboxylesterase, a caspase, a diaphorase, a histone deacetylase, a legumain, or matrix metalloproteinase. In some embodiments, the enzyme labile group is a group cleavable by cathepsin B.

In a 17^{th} embodiment of the second aspect, for the linker-photosensitizer compound of formula (II), (IIA), (IIB) or a pharmaceutically acceptable salt thereof, the spacer represented by L^{P} comprises a peptide and a self-immolative group; and the remaining variables are as defined in the 1^{st}, 2^{nd}, 3^{rd}, 4^{th}, 5^{th}, 6^{th}, 7^{th}, 8^{th}, 9^{th}, 10^{th}, 11^{th} or 12^{th} embodiment of the second aspect or any embodiments described therein.

In a 18^{th} embodiment of the second aspect, for the linker-photosensitizer compound of formula (II), (IIA), (IIB) or a pharmaceutically acceptable salt thereof, the spacer L^{P} is presented by the following formula:

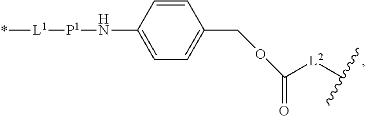

wherein:
*- represents a bond to the Linker group;

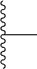

represents a bond to the group Z;
L¹ and L² are each independently a connecting spacer;
P¹ is a peptide comprising 2 to 5 amino acid residues; and the remaining variables are as defined in the 17^{th} embodiment of the second aspect or any embodiments described therein.

In a 19^{th} embodiment of the second aspect, for the linker-photosensitizer compound of formula (II), (IIA), (IIB) or a pharmaceutically acceptable salt thereof, P¹ is a peptide selected from Phe-Arg-Arg-Gly, Glu-Val-Cit, Val-Cit, Cit-Val, Gly-Gly-Phe, Val-Ala, or Ala-Val; and the remaining variables are as defined in the 18^{th} embodiment of the second aspect.

In a 20^{th} embodiment of the second aspect, for the linker-photosensitizer compound of formula (II), (IIA), (IIB) or a pharmaceutically acceptable salt thereof, P¹ is:

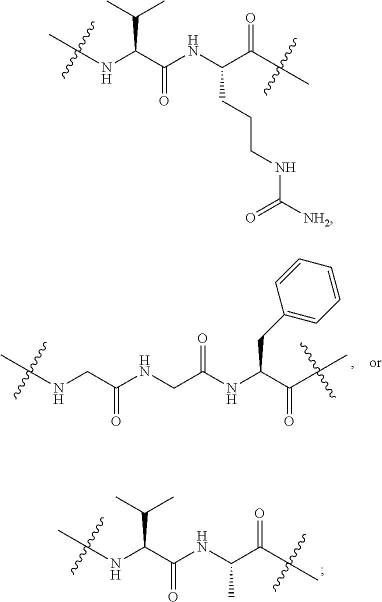

and the remaining variables are as defined in the 18^{th} embodiment of the second aspect.

In a 21^{st} embodiment of the second aspect, for the linker-photosensitizer compound of formula (II), (IIA), (IIB) or a pharmaceutically acceptable salt thereof, L¹ comprises a hydrophilic moiety; and the remainding variables are as defined in the 18^{th}, 19^{th} or 20^{th} embodiment of the second aspect. In some embodiment, L¹ comprises a PEG moiety.

In a 22^{nd} embodiment of the second aspect, for the linker-photosensitizer compound of formula (II), (IIA), (IIB) or a pharmaceutically acceptable salt thereof, L¹ is *—CH₂—CH₂—(OCH₂CH₂)ₙ—C(═O)—**; n is an integer from 0 to 30; *- represents a bond to the Linker group; **- represents a bond to P¹; and the remainding variables are as defined in the 18^{th}19^{th} or 20^{th} embodiment of the second aspect. In some embodiments, n is an integer from 2 to 16. In some embodiments, n is an integer from 2 to 10. In some embodiment, n is an integer from 2 to 8. In some embodiments, n is an integer from 3 to 5. In some embodiments, n is 2, 3, 4, 5, 6, 7 or 8.

In a 23^{rd} embodiment of the second aspect, for the linker-photosensitizer compound of formula (II), (IIA), (IIB) or a pharmaceutically acceptable salt thereof, L¹ is *—CH₂—CH₂—(OCH₂CH₂)₃—C(═O)—**, wherein *- represents a bond to the Linker group; and **- represents a bond to P¹; and the remainding variables are as defined in the 18^{th}, 19^{th} or 20^{th} embodiment of the second aspect.

In a 24^{th} embodiment of the second aspect, for the linker-photosensitizer compound of formula (II), (IIA), (IIB) or a pharmaceutically acceptable salt thereof, L² is —NH—C₁₋₆alkyl-**, wherein ** is the site connected to Z; and the remainding variables are as defined in the 18^{th}, 19^{th}, 20^{th}, 21^{st}, 22^{nd} or 23^{rd} embodiment of the second aspect or any embodiments described therein.

In a 25^{th} embodiment of the second aspect, for the linker-photosensitizer compound of formula (II), (IIA), (IIB) or a pharmaceutically acceptable salt thereof, L² is —NH—CH₂CH₂—**, wherein ** is the site connected to Z; and the remainding variables are as defined in the 18^{th}, 19^{th}, 20^{th}, 21^{st}, 22^{nd} or 23^{rd} embodiment of the second aspect or any embodiments described therein.

In a 26^{th} embodiment of the second aspect, the linker-photosensitizer compound of the present disclosure is represented by the following formula:

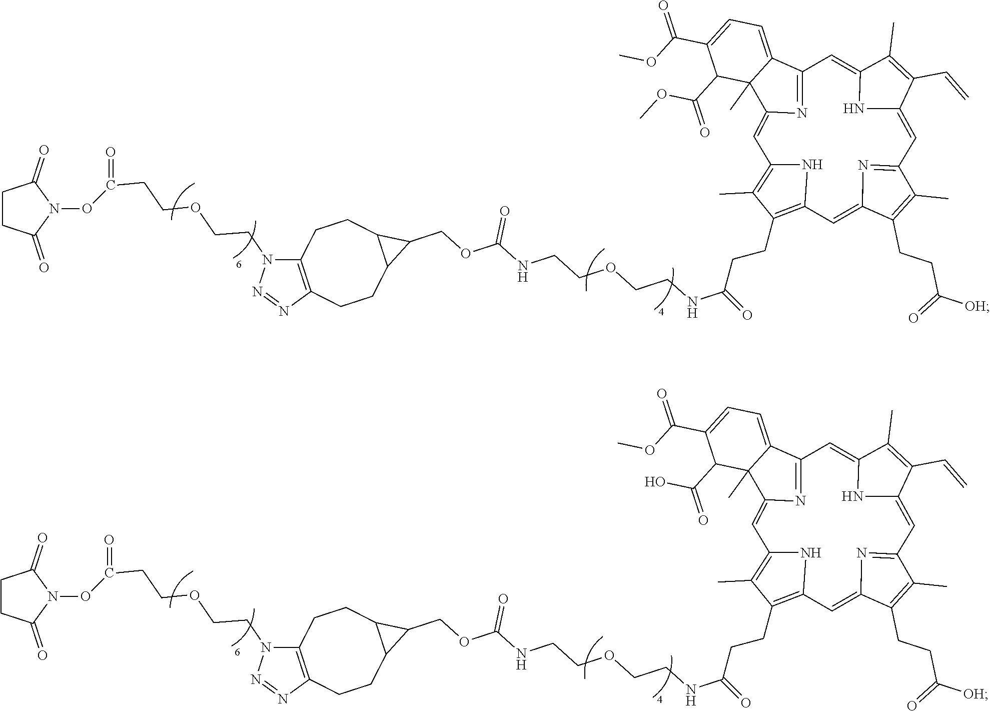

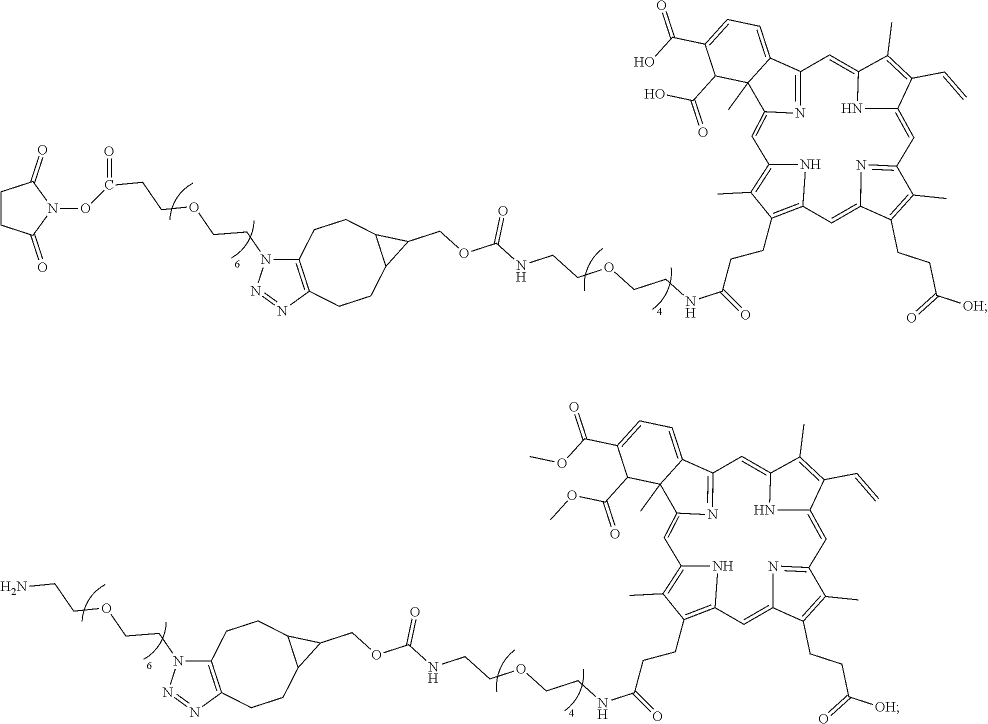

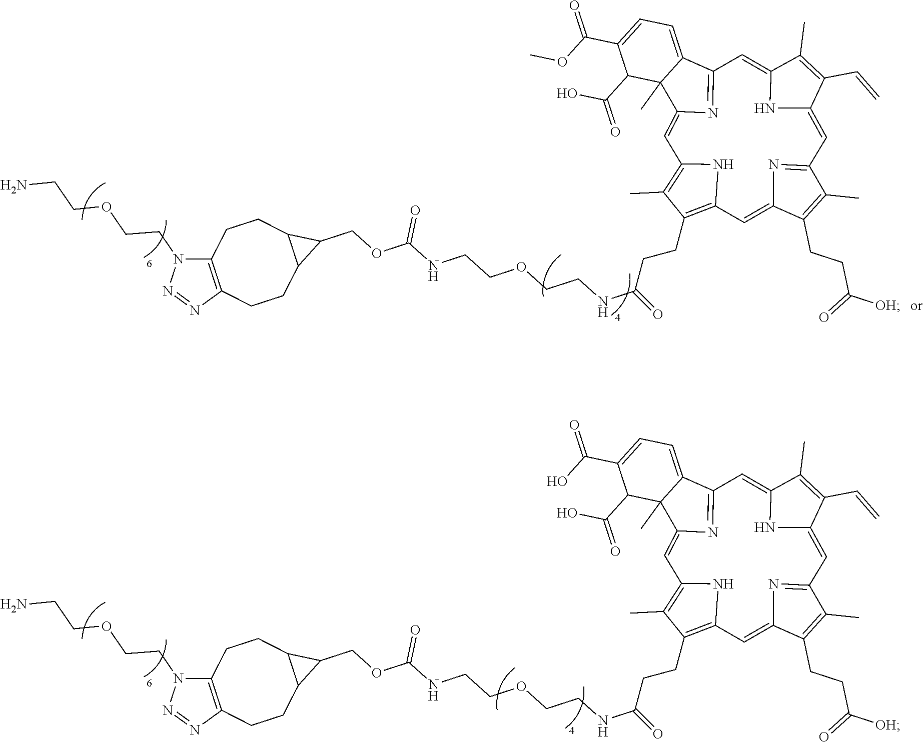

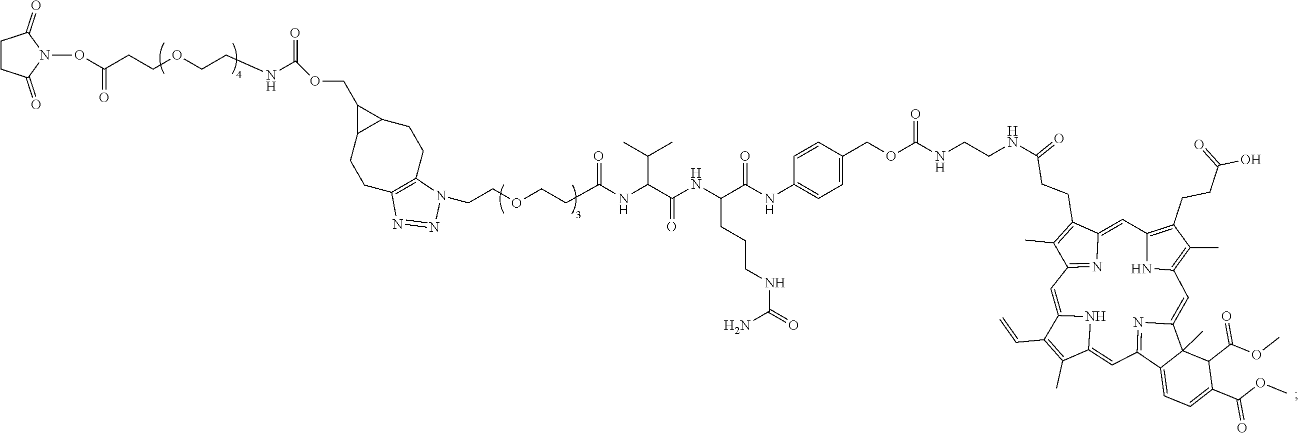

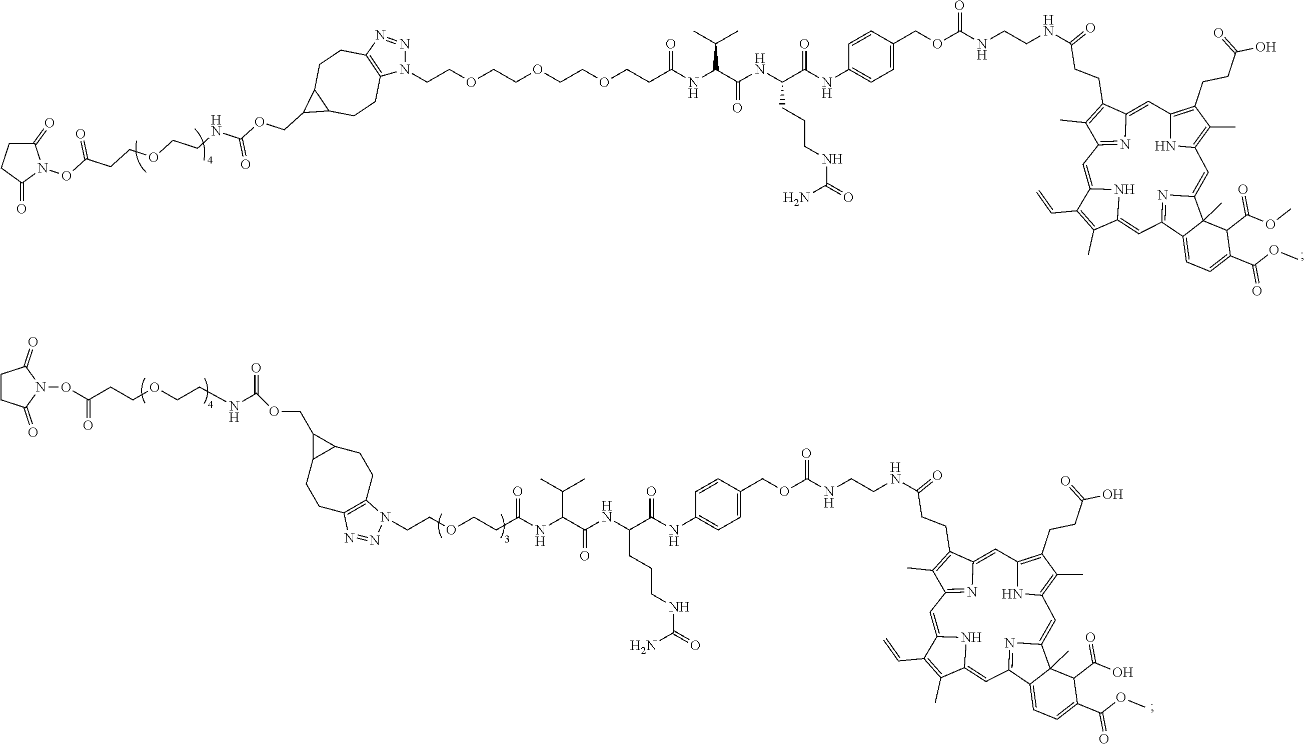

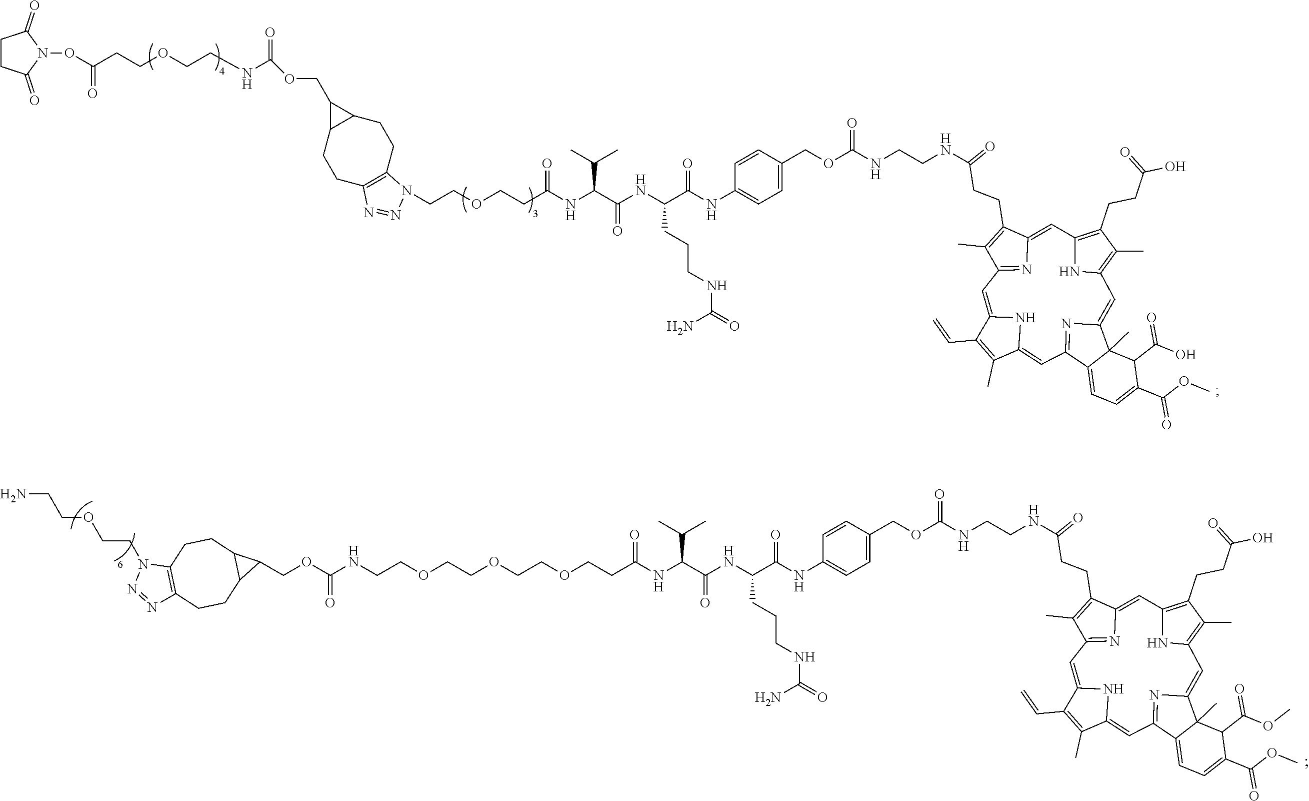

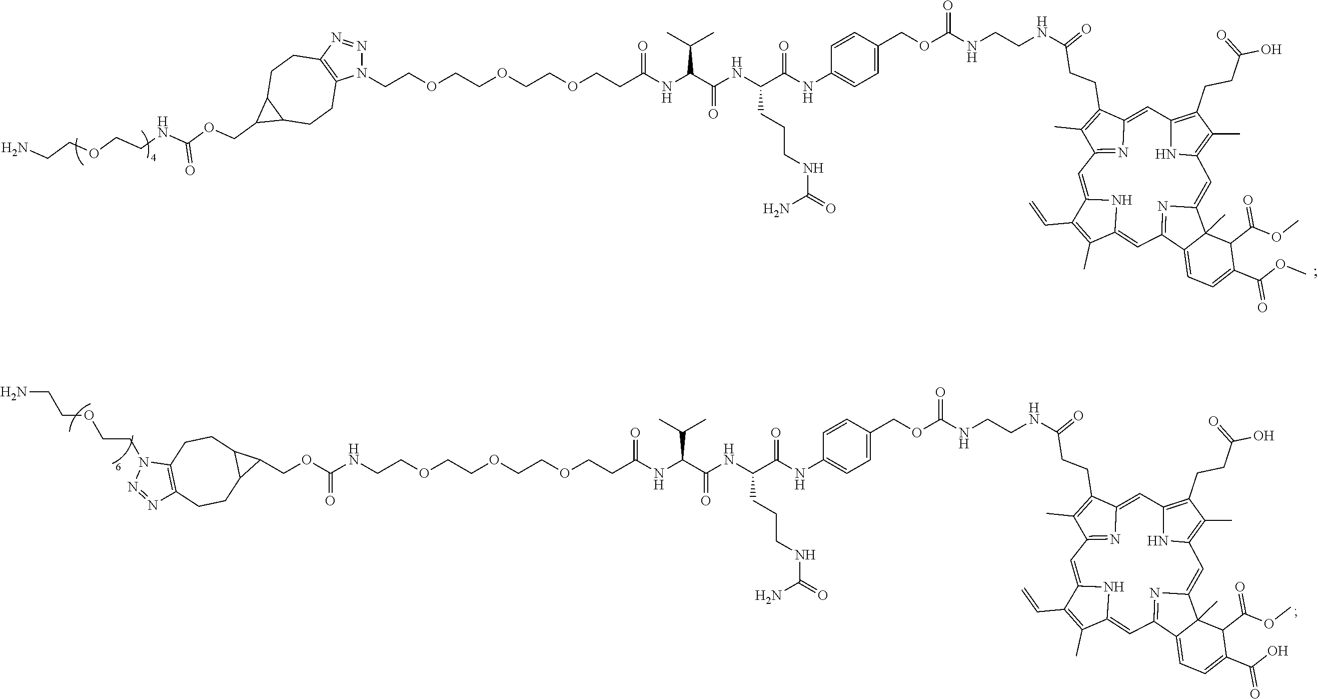

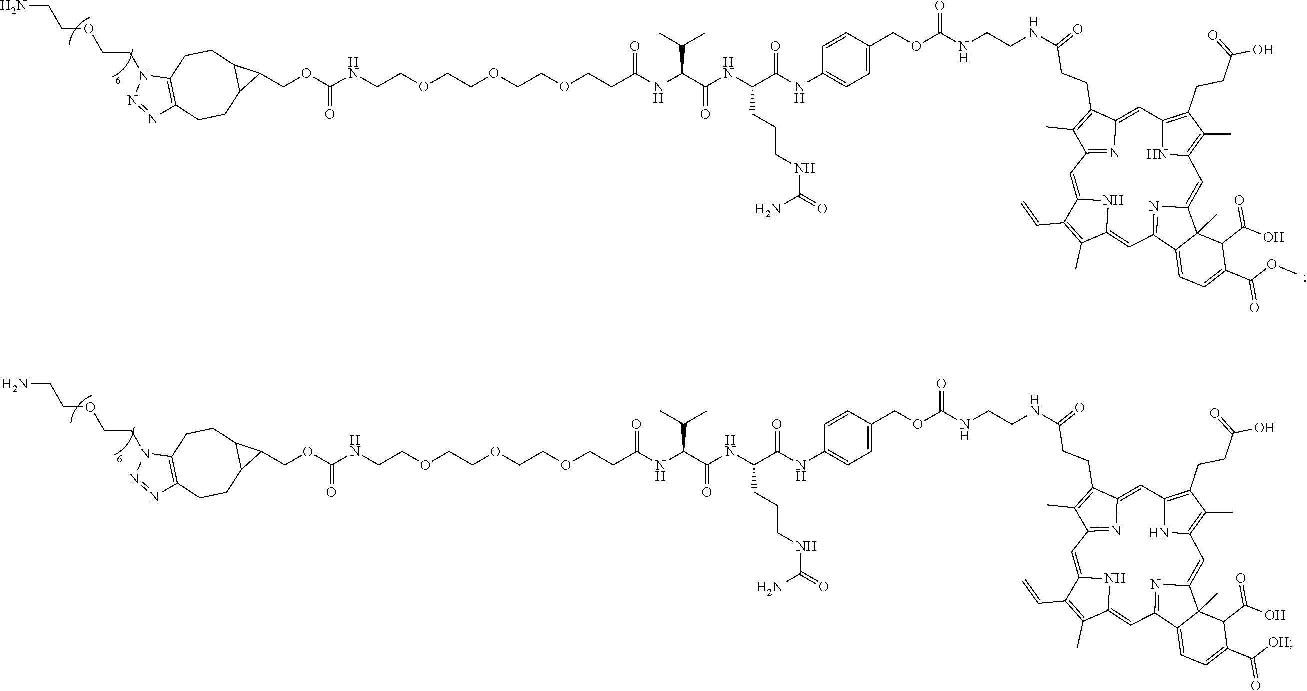

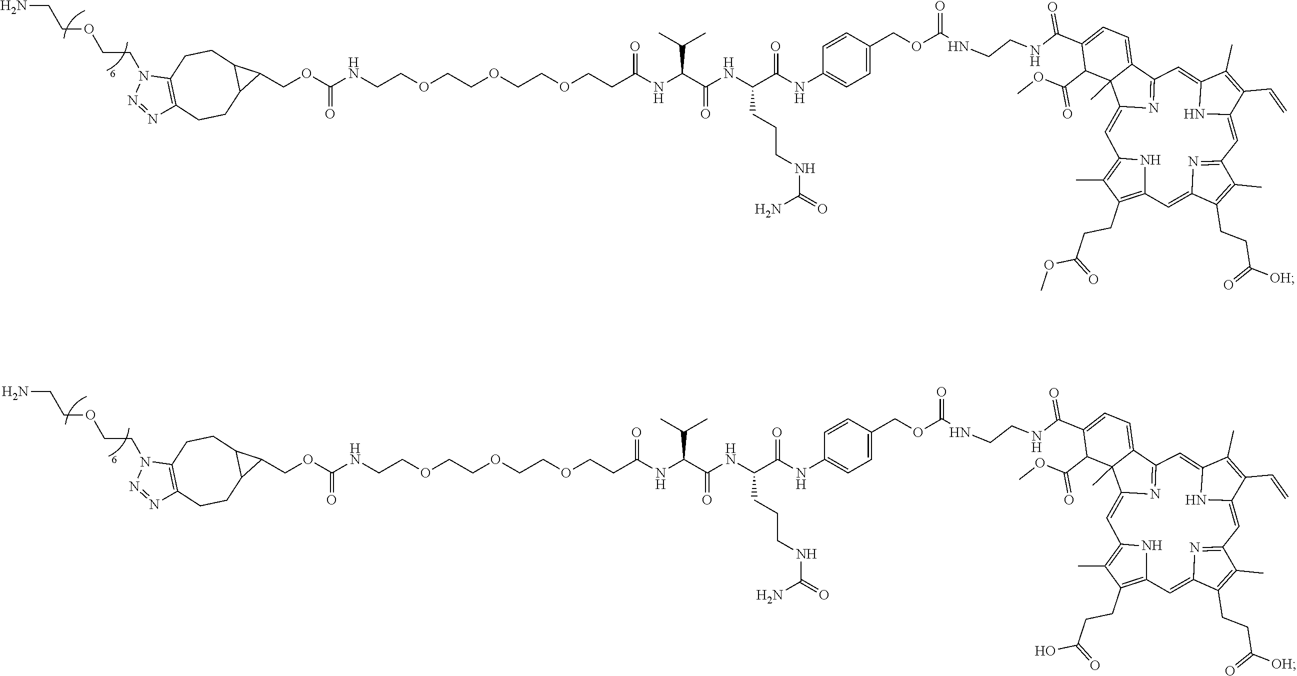

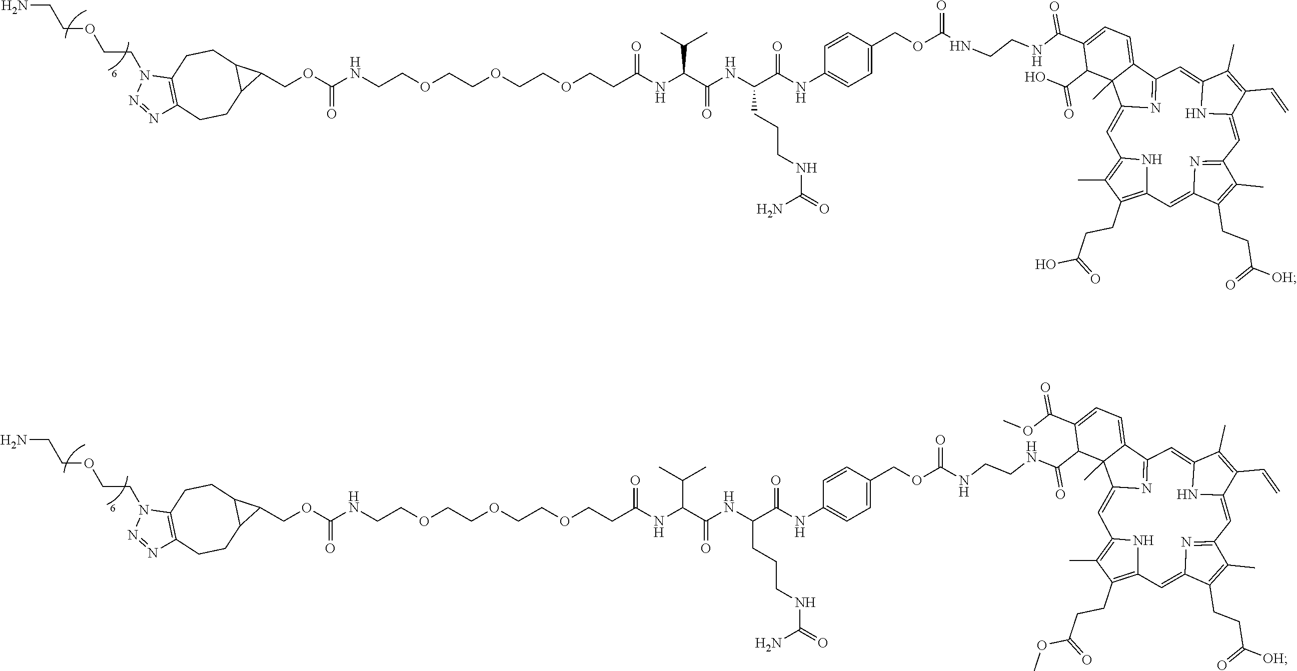

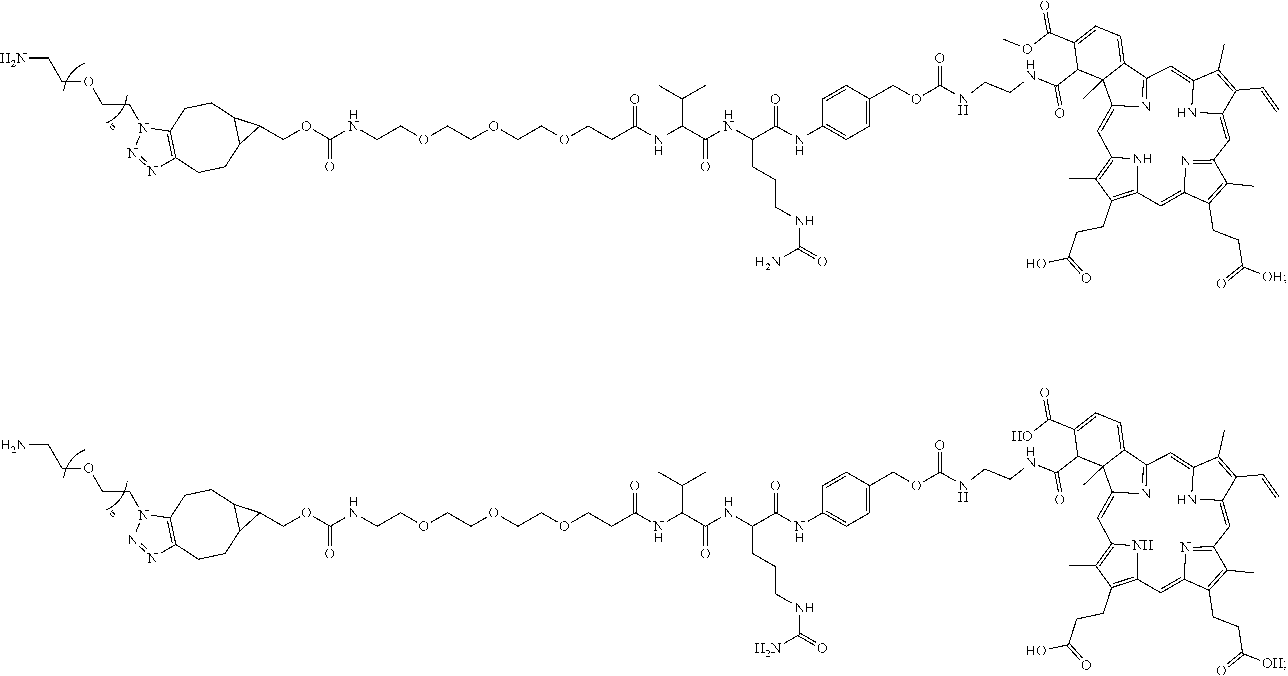

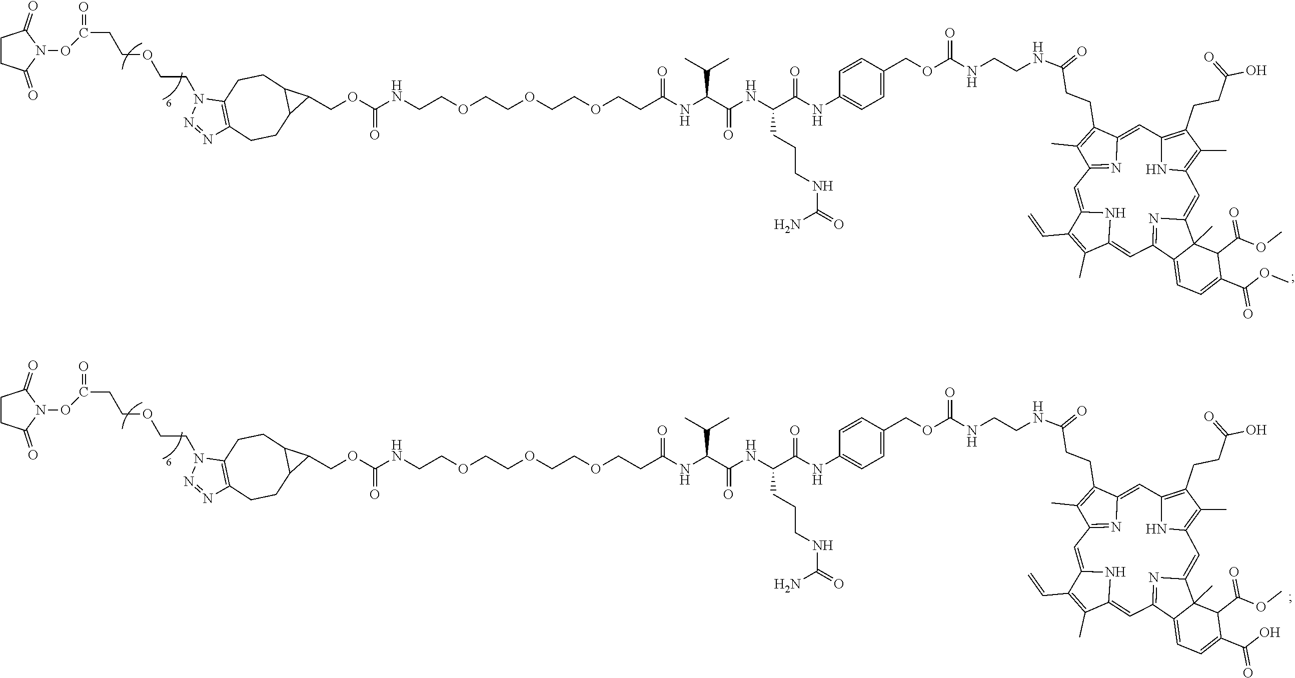

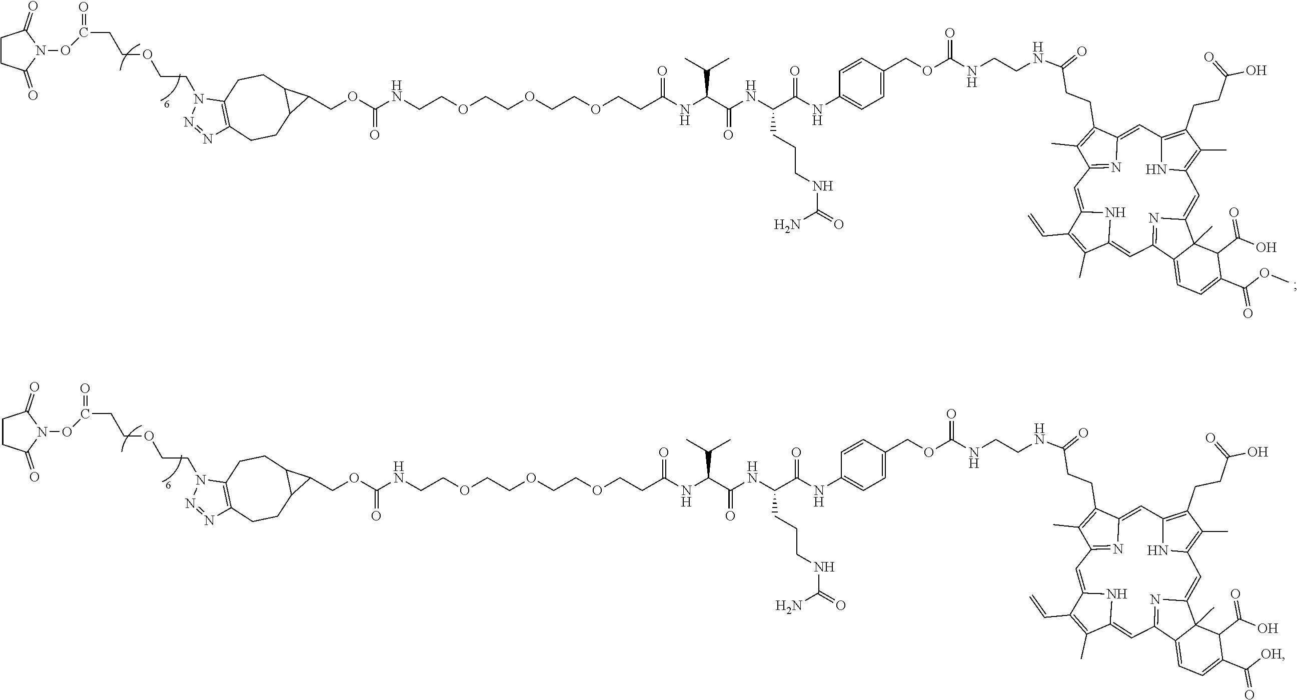

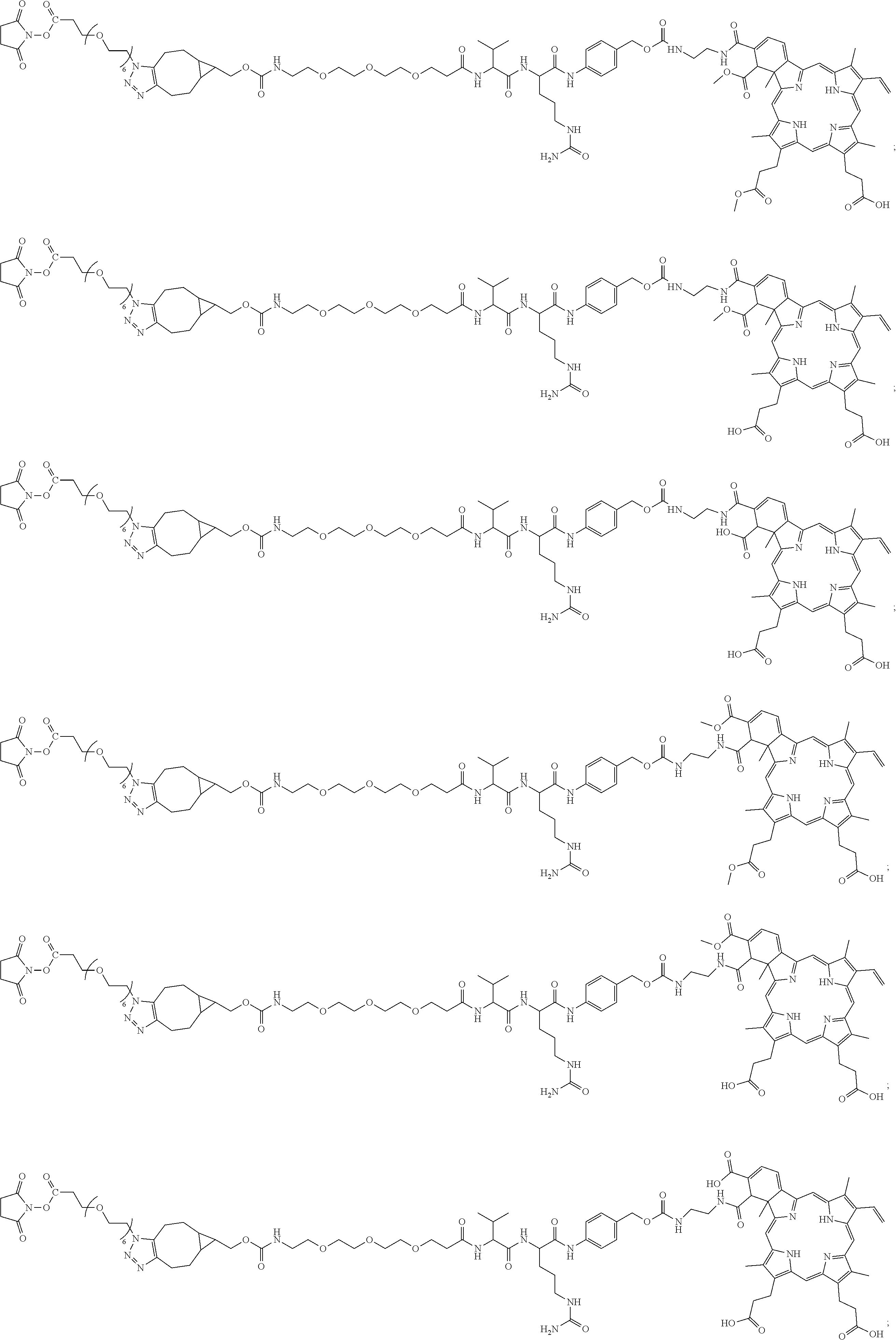

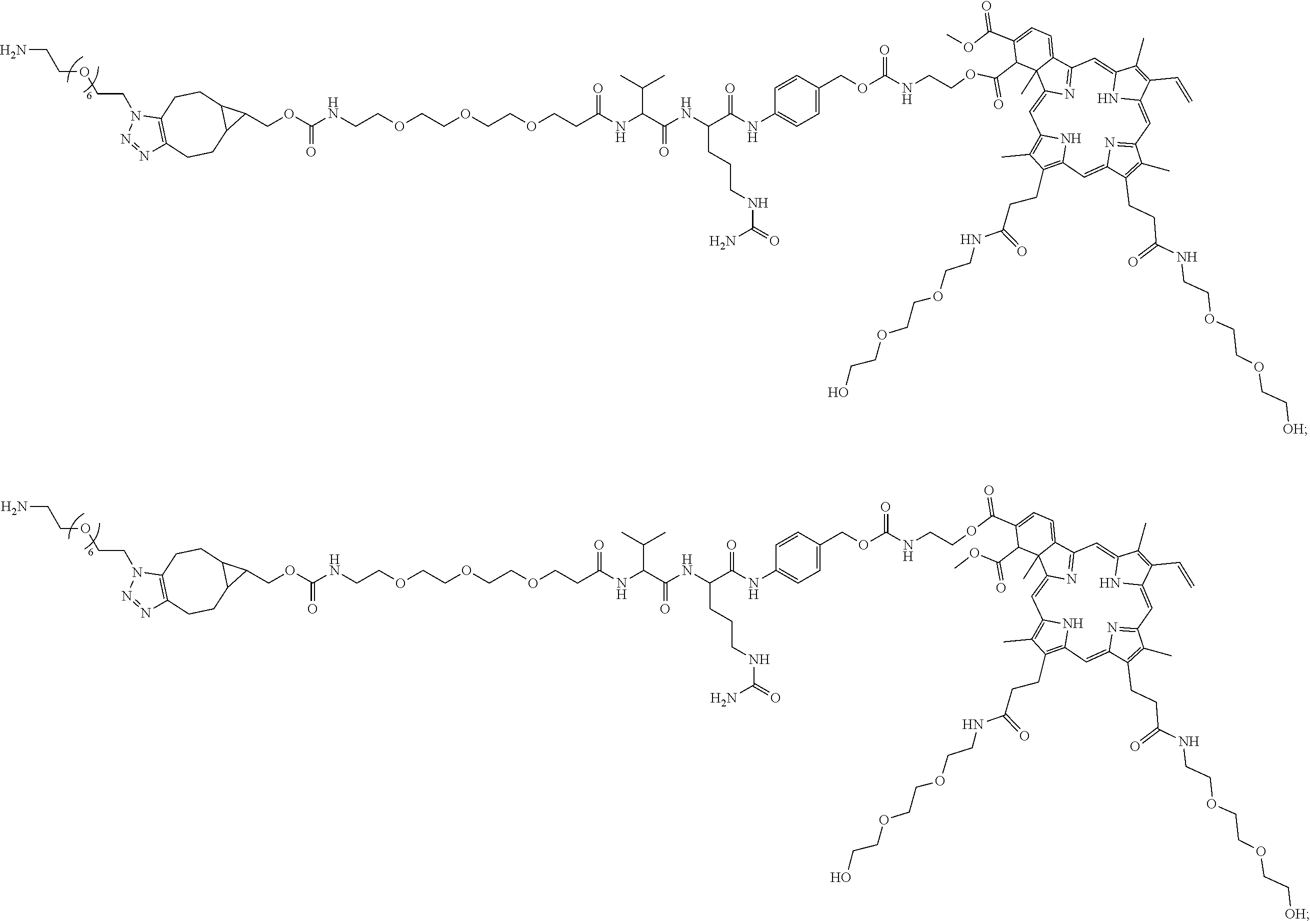

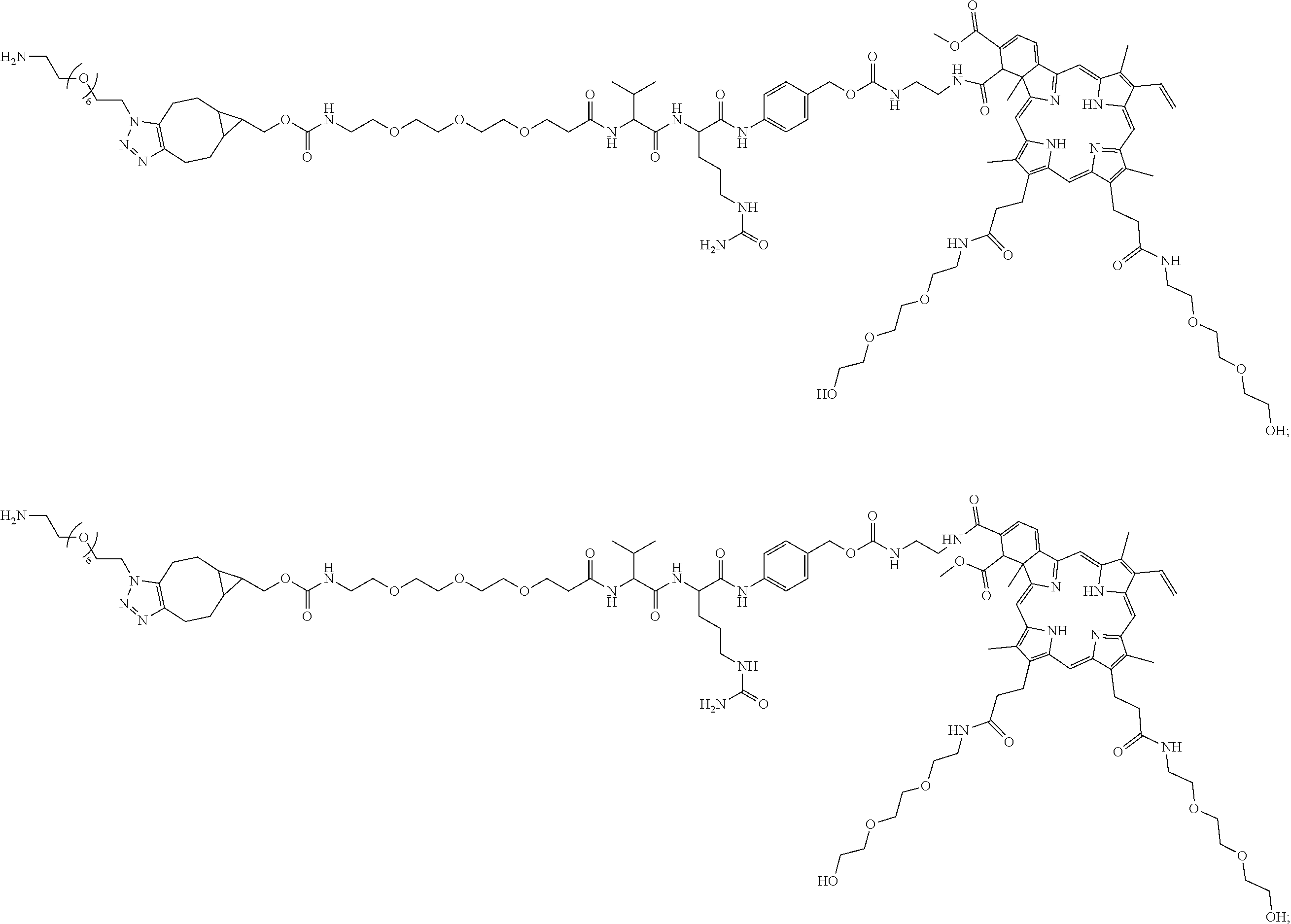

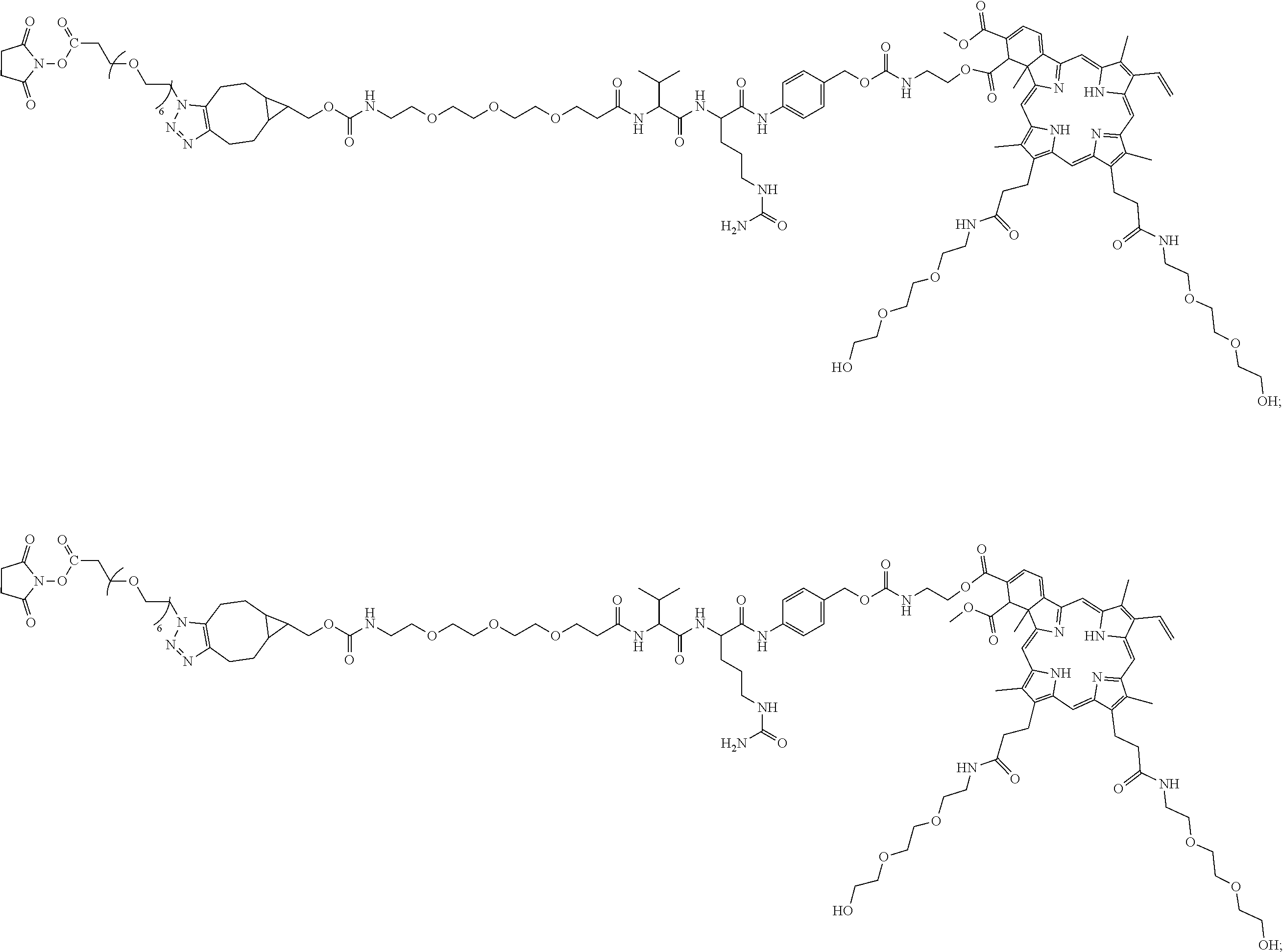

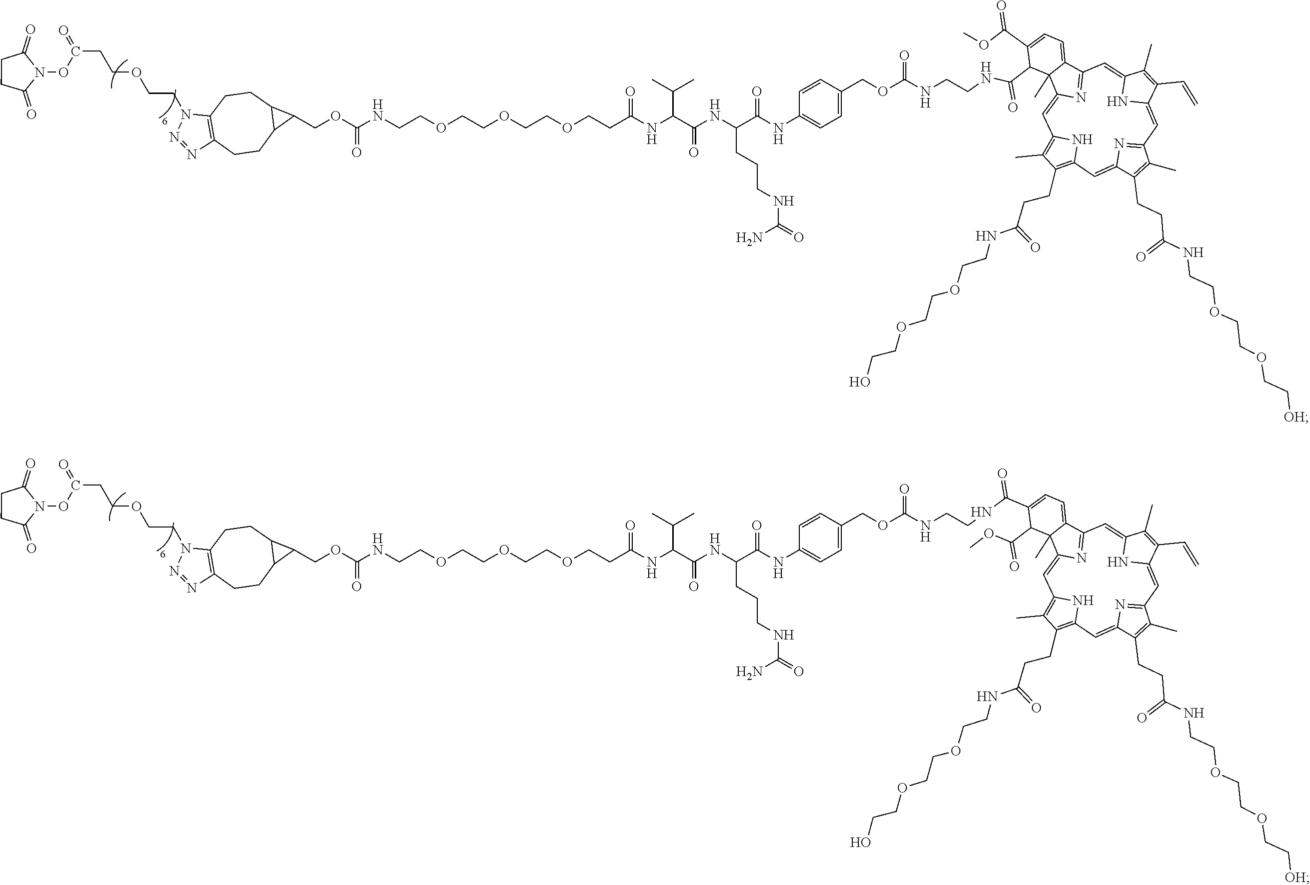

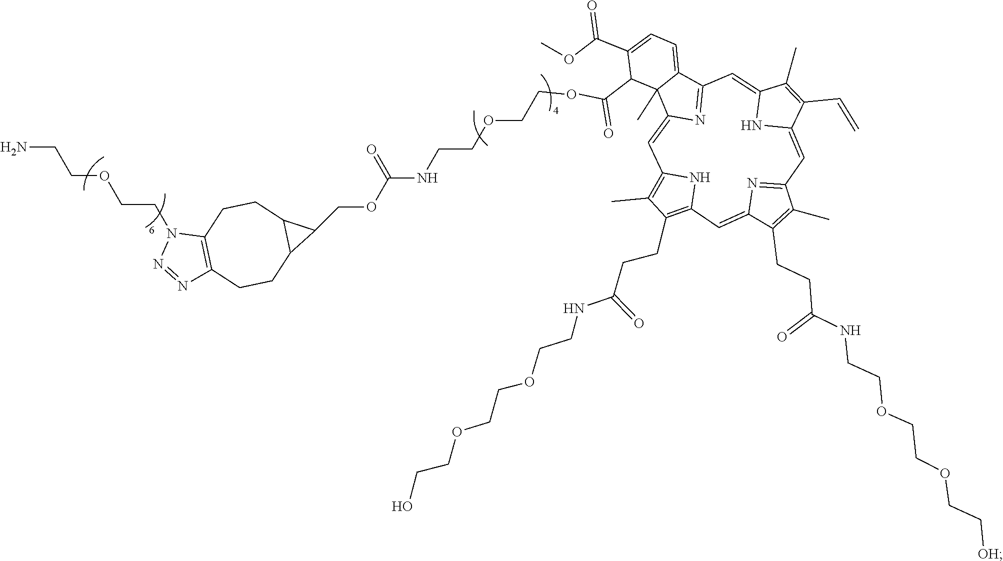

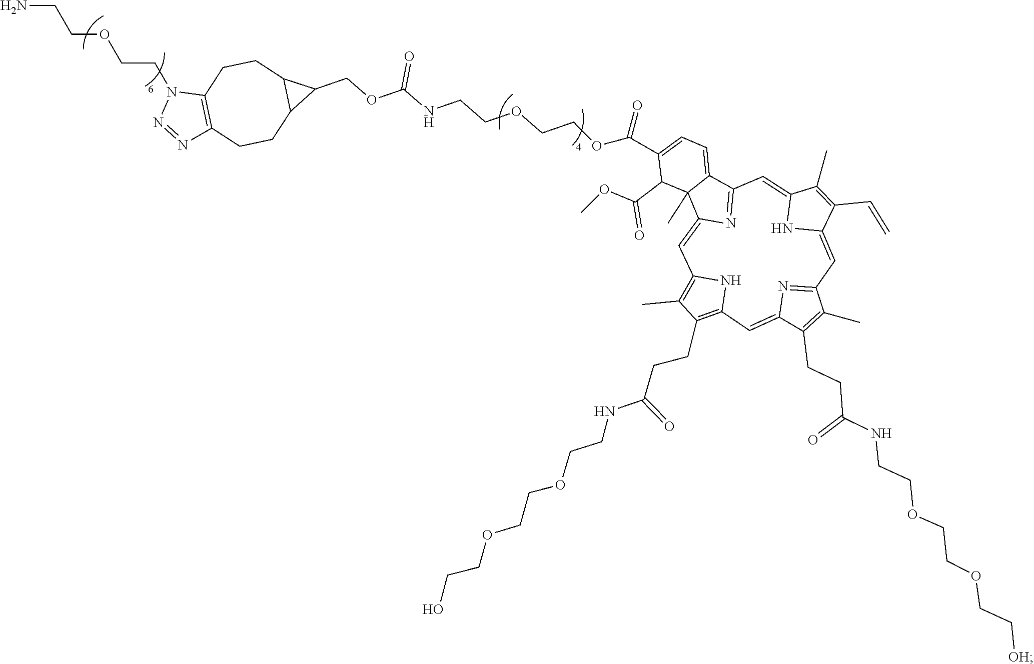

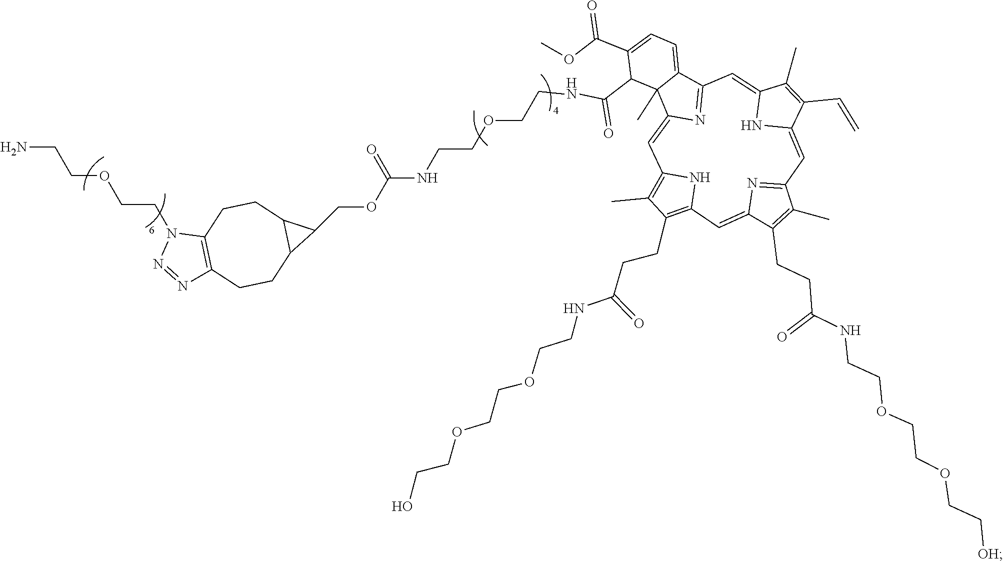

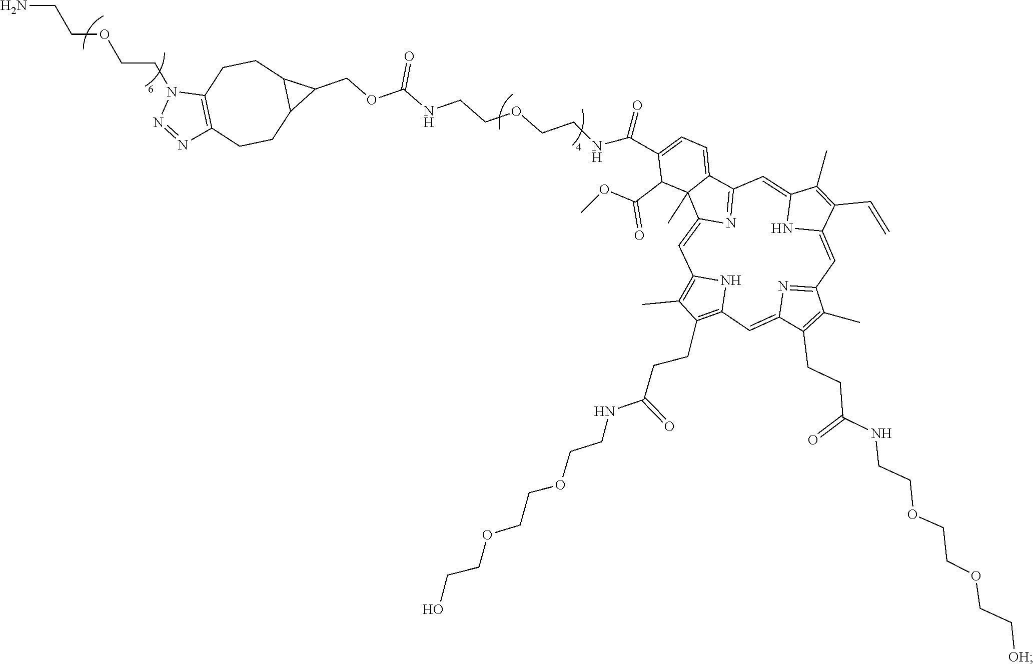

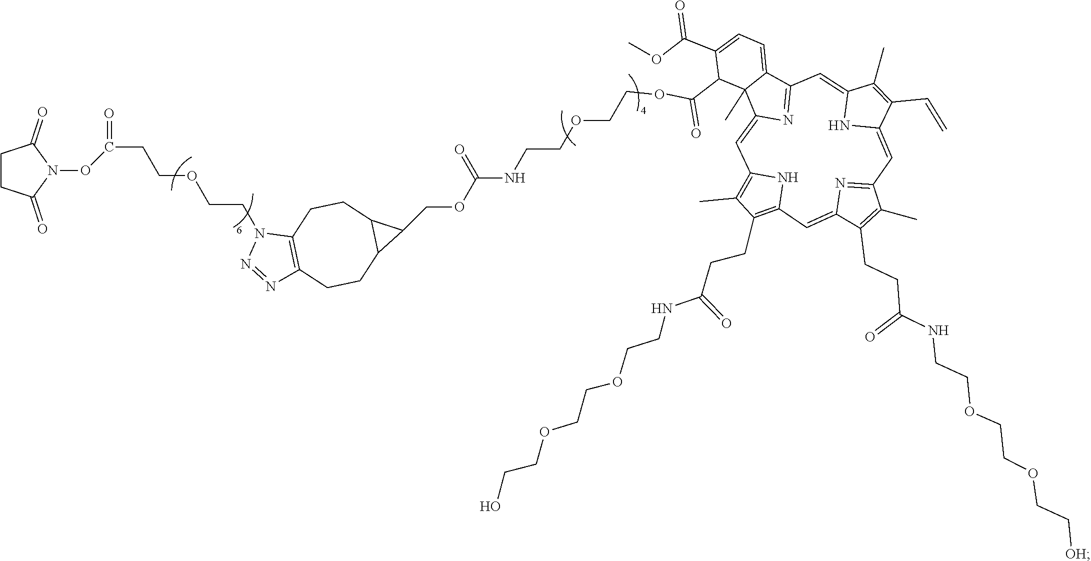

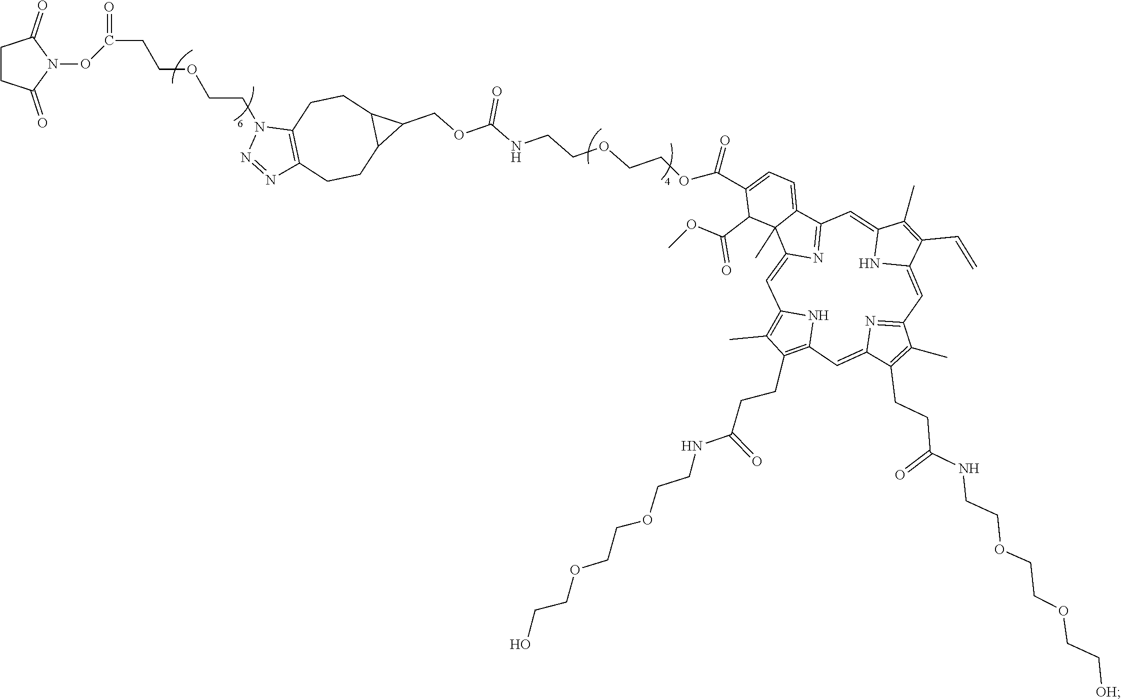

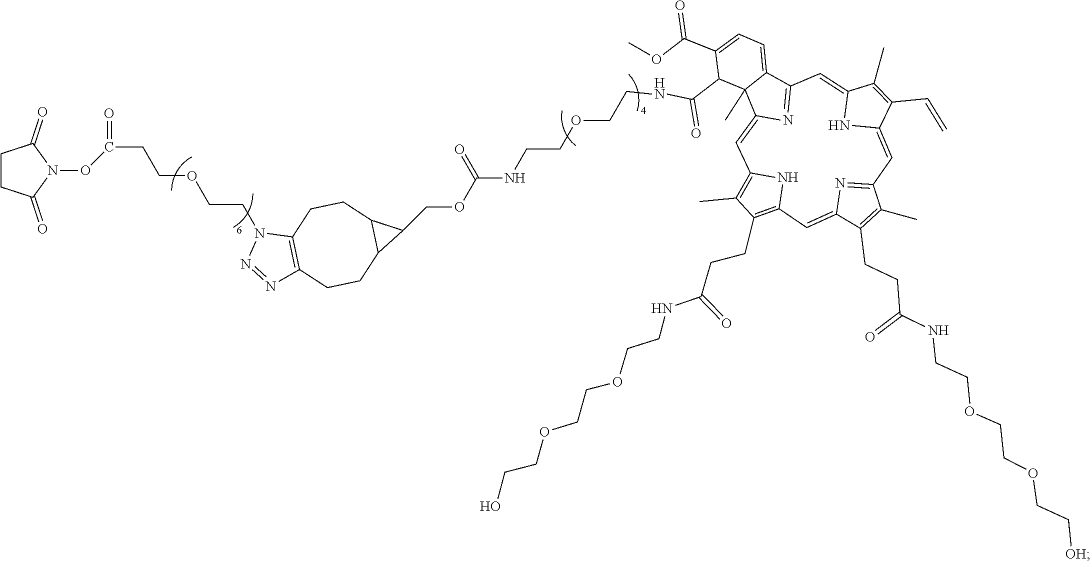

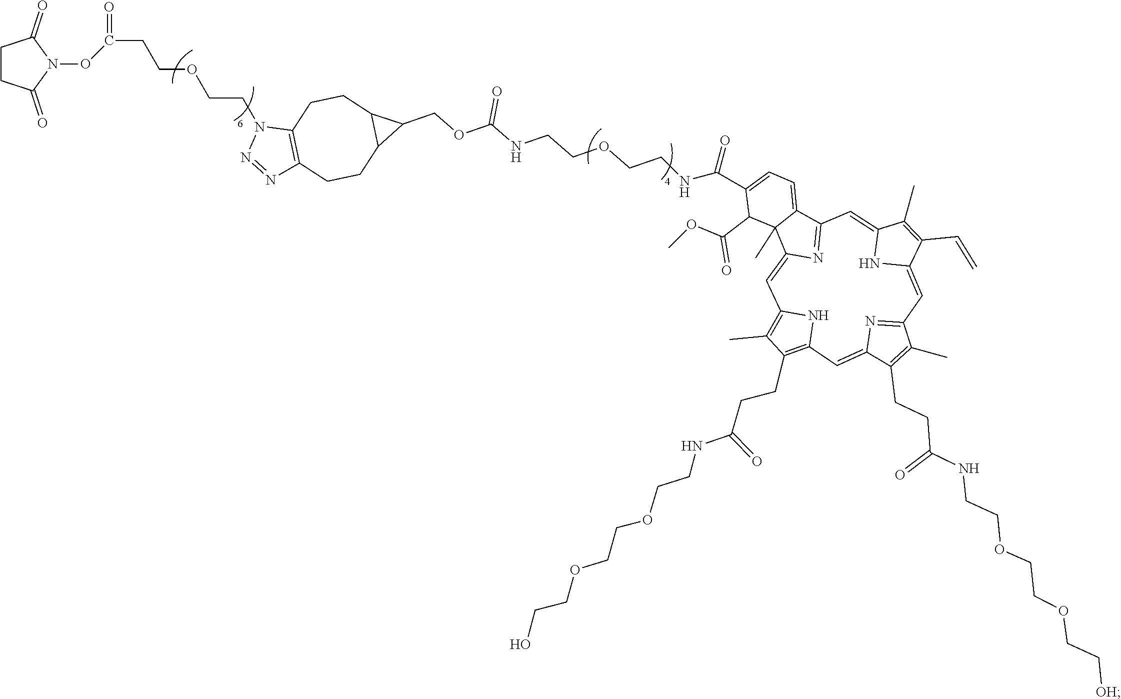

or a pharmaceutically acceptable salt thereof, a regioisomer, or a pharmaceutically acceptable salt of the regioisomer.
Modified Targeting Agents with Reactive Groups

In a third aspect, the present disclosure provides modified targeting agents having reactive groups that can be covalently attached to the photosentizer compounds described in the first aspect or the linker-photosensitizer compounds described in the second aspect. In a 1st embodiment of the third aspect, the modified targeting agent is represented by formula (V):

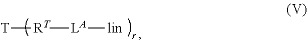

or a pharmaceutically acceptable salt thereof, wherein:
T is a targeting agent;
R^{T} is a reactive group covalently linked to the targeting agent;
L^{A} is a spacer;
lin is a reactive group; and
r is an integer from 1 to 20.

In a 2^{nd} embodiment, for the modified targeting agent of formula (V), R^{T} is —NH—, —C(═O)—, or

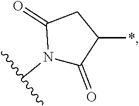

wherein * is the site connected to the targeting agent; and the remaining variables are as described in the 1^{st} embodiment of the third aspect.

In a 3^{rd} embodiment, for the modified targeting agent of formula (V), R^{T} is —NH— or —C(═O)—, wherein * is the site connected to the targeting agent; and the remaining variables are as described in the 1st embodiment of the third aspect.

In a 4^{th} embodiment, for the modified targeting agent of formula (V), L^{A} is a spacer comprising a hydrophilic moiety; and the remaining variables are as described in the 1^{st}, 2^{nd} or 3^{rd} embodiment of the third aspect. In some embodiments, L^{A} is a spacer comprising a PEG moiety.

In a 5^{th} embodiment, for the modified targeting agent of formula (V), L^{A} is * CH₂CH₂(OCH₂CH₂)ₚ—, wherein * is the site connected to R^{T}; n is an integer from 1 to 10; and p is an integer from 0 to 30; and the remaining variables are as described in the 1^{st}, 2^{nd} or 3rd embodiment of the third aspect. In some embodiments, p is an integer from 2 to 8. In some embodiments, p is an integer from 3 to 5. In some embodiments, p is 2, 3, 4, 5, 6, 7 or 8. In some embodiments, p is 6.

In a5^{th} embodiment, for the modified targeting agent of formula (V), lin in formula (V) is

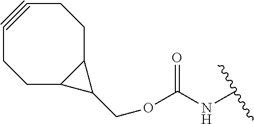 [SMILES: *NC(=O)OCC1C2CCC#CCCC21]

or N₃; and the remaining variables are as described in the 1^{st}2^{nd}, 3^{rd} or 4^{th} embodiment of the third aspect.

In a 6^{th} embodiment, the modified targeting agent is represented by the following formula:

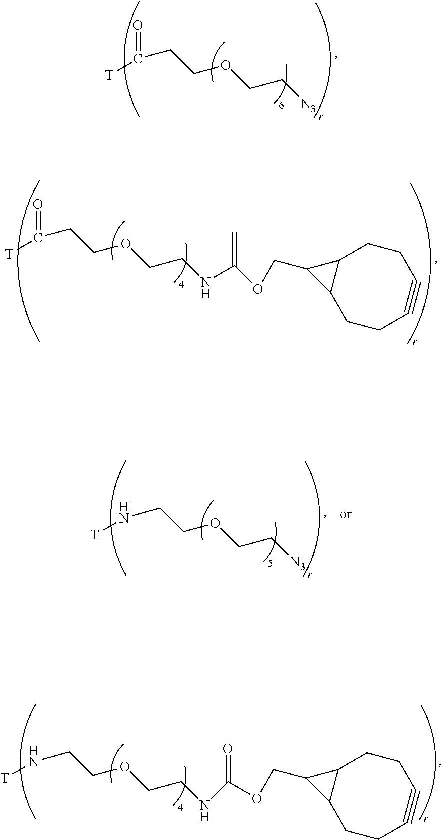

or a pharmaceutically acceptable salt thereof.

### Photosensitizer-Targeting Agent Conjugates

In a fourth aspect, the present disclosure provides a conjugate comprising a targeting agent covalently linked to a photosensitizer, wherein the photosensitizer is a benzoporphyrin analog. In a 1^{st} embodiment of the fourth aspect, the conjugate of the present disclosure is represented by formula (III):

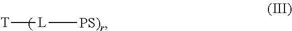

or a pharmaceutically acceptable salt thereof, wherein:
T is targeting agent;
L is a spacer connecting the targeting agent and PS;
r is an integer from 1 to 20;
PS is a photosensitizer that is a benzoporphyrin analog represented by the following formula:

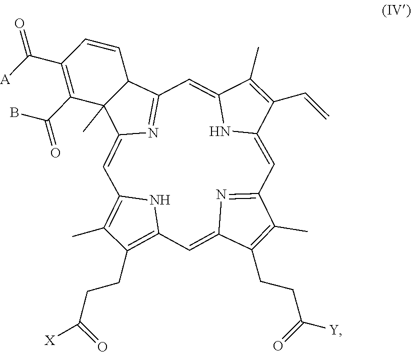

or a pharmaceutically acceptable salt thereof, wherein:
A, B, X and Y are each independently —OH, —OC₁₋₄alkyl, —N(R¹⁰⁰)(R¹⁰¹) or a bond covalently linked to L, provided one of A, B, X and Y is a bond covalently linked to L;
R¹⁰⁰ is H or C₁₋₃alkyl;
R¹⁰¹ is H, C₁₋₃alkyl or —(CH₂—CH₂—O)ₙᵣR¹⁰²;
R¹⁰² is H or Me;
nr is an integer from 1 to 16.

In a 2^{nd} embodiment of the fourth aspect, for the conjugate of formula (III) or a pharmaceutical acceptable salt thereof, -L-PS in formula (III) is represented by the following formula:

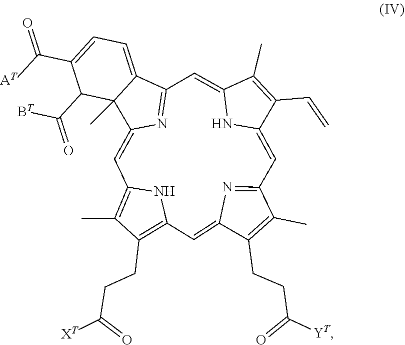

or a pharmaceutically acceptable salt thereof, wherein:
A^{T}, B^{T}, X^{T} and Y^{T} are each independently —OH, —OC₁₋₄alkyl, —N(R¹⁰⁰)(R¹⁰¹) or —Z-L^{P}-linker-L^{A}-R^{T}, provided one of A^{T}, B^{T}, X^{T} and Y^{T} is —Z-L^{P}-linker-L^{A}-R^{T}; Z is —O— or —NR¹—;
R¹ is H or C₁₋₃alkyl;
L^{P} and L^{A} are each independently a spacer;
Linker is connection moiety formed by a first reactive group attached to L and a second reactive group attached to L^{A};
R^{T} is a reactive group covalently linked to the targeting agent; and the remaining variables are as defined in the 1^{st} embodiment of the fourth aspect.

In a 3^{rd} embodiment of the fourth aspect, for the conjugate of formula (III) or a pharmaceutically acceptable salt thereof, -L-PS is represented by formula (IV), R^{T} is —C(O)—, —NH—, or

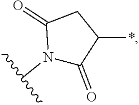

wherein *- represents a bond to the targeting agent and

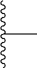

represents a bond to L^{A}; and the remaining variables are as defined in the 2^{nd} embodiment of the fourth aspect.

In a 4^{th} embodiment of the fourth aspect, for the conjugate of formula (III) or a pharmaceutically acceptable salt thereof, -L-PS is represented by formula (IV), R^{T} is —C(O)— or —NH—, wherein *- represents a bond to the targeting agent and

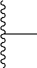

represents a bond to L^{A}; and the remaining variables are as defined in the 2^{nd} embodiment of the fourth aspect.

In a 5^{th} embodiment of the fourth aspect, for the conjugate of formula (III) or a pharmaceutically acceptable salt thereof, -L-PS is represented by formula (IV), L^{A} is a spacer comprising a hydrophilic moiety; and the remaining variables are as defined in the 2^{nd}, 3^{rd} or 4^{th} embodiment of the fourth aspect. In some embodiments, L^{A} comprises a PEG moiety.

In a 6^{th} embodiment of the fourth aspect, for the conjugate of formula (III) or a pharmaceutically acceptable salt thereof, -L-PS is represented by formula (IV), L^{A} is *- CH₂CH₂(OCH₂CH₂)ₚ—, wherein * is the site connected to R^{T}; and p is an integer from 0 to 30; and the remaining variables are as defined in the 2^{nd}, 3^{rd} or 4^{th} embodiment of the fourth aspect or any embodiments described therein. In some embodiments, p is an integer from 2 to 10. In some embodiments, p is an integer from 2 to 8. In some embodiments, p is an integer from 3 to 5. In some embodiments, p is 2, 3, 4, 5, 6, 7 or 8. In some embodiments, p is 6.

In a 7^{th} embodiment of the fourth aspect, for the conjugate of formula (III) or a pharmaceutically acceptable salt thereof, -L-PS is represented by formula (IV), Linker in formula (IV) is formed by a first reactive group attached to L^{P} and a second reactive group attached to L^{A}, wherein the first reactive group and the second reactive group are each selected from a maleimide group, a thiol group, a cyclooctyne group, and an azido group; and the remaining variables are as defined in the 2^{nd}, 3^{rd} 4^{th}, 5^{th} or 6^{th} embodiment of the fourth aspect or any embodiments described therein or any embodiments described therein.

In a 8^{th} embodiment of the fourth aspect, for the conjugate of formula (III) or a pharmaceutically acceptable salt thereof, -L-PS is represented by formula (IV), Linker in formula (IV) is represented by the following formula:

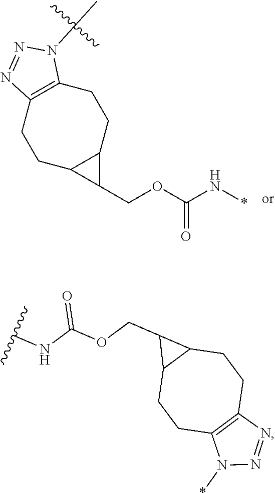 [SMILES: *NC(=O)OCC1C2CCc3nnn(*)c3CCC21 ; *NC(=O)OCC1C2CCc3nnn(*)c3CCC21]

wherein *- represents a bond to L^{P} and

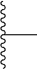

represents a bond to L^{A}; and the remaining variables are as defined in the 2^{nd}, 3^{rd}, 4^{th}, 5^{th} or 6^{th} embodiment of the fourth aspect or any embodiments described therein.

In a 9^{th} embodiment of the fourth aspect, for the conjugate of formula (III) or a pharmaceutically acceptable salt thereof, -L-PS is represented by formula (IV) and A^{T}, B^{T}X^{T} and Y^{T} are defined as follows:
a) A^{T} and B^{T} are —OCH₃; and one of X^{T} and Y^{T} is —OCH₃ and the other one of X^{T} and YT is —Z-L^{P}-linker-L^{A}-R^{T}.
b) A^{T} and B^{T} are —OCH₃; and one of X^{T} and Y^{T} is —OH and the other one of X^{T} and YT is —Z-L^{P}-linker-L^{A}-R^{T}.
c) one of A^{T} and B^{T} is —OCH₃, and the other one of A^{T} and B^{T} is —OH; and one of X^{T} and Y^{T} is —OH and the other one of X^{T} and Y^{T} is —Z-L^{P}-linker-L^{A}-R^{T};
d) A^{T} and B^{T} are —OH; and one of X and Y is —OH and the other one of X^{T} and Y^{T} is —Z-L^{P}-linker-L^{A}-R^{T}; or
e) one of A^{T} and B^{T} is —Z-L^{P}-linker-L^{A}-R^{T} and the other one of A^{T} and B^{T} is —OH or —OCH₃; and X^{T} and Y^{T} are both —NH(CH₂—CH₂—O)ₙᵣR¹⁰², wherein R¹⁰² is H or Me; and nr is an integer from 2 to 8; and the remaining variables are as defined in the 2^{nd}, 3^{rd}, 4^{th}, 5^{th}, 6^{th} 7^{th} or 8^{th} embodiment of the fourth aspect or any embodiments described therein.

In some embodiments, A^{T} and B^{T} are —OCH₃; and one of X^{T} and Y^{T} is —OCH₃ and the other one of X^{T} and Y^{T} is —Z-L^{P}-linker-L^{A}-R^{T}.

In some embodiments, A^{T} and B^{T} are —OCH₃; and one of X^{T} and Y^{T} is —OH and the other one of X^{T} and Y^{T} is —Z-L^{P}-linker-L^{A}-R^{T}.

In some embodiments, one of A^{T} and B^{T} is —OCH₃, and the other one of A^{T} and B^{T} is —OH; and one of X^{T} and Y^{T} is —OH and the other one of X^{T} and Y^{T} is —Z-L^{P}-linker-L^{A}-R^{T}.

In some embodiments, A^{T} and B^{T} are —OH; and one of X and Y is —OH and the other one of X^{T} and Y^{T} is —Z-L^{P}-linker-L^{A}-R^{T}.

In some embodiments, one of A^{T} and B^{T} is —Z-L^{P}-linker-L^{A}-R^{T} and the other one of A^{T} and B^{T} is —OH or —OCH₃; and X^{T} and Y^{T} are both —NH(CH₂—CH₂—O)ₙᵣR¹⁰², wherein R¹⁰² is H or Me; and nr is an integer from 2 to 8.

In a 10^{th} embodiment of the fourth aspect, for the conjugate of formula (III) or a pharmaceutically acceptable salt thereof, -L-PS is represented by formula (IVA):

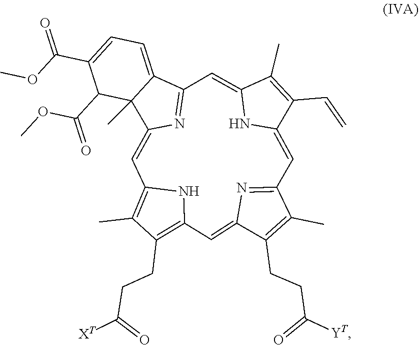

or a pharmaceutically acceptable salt thereof, wherein one of X^{T} and Y^{T} is —Z-L^{P}-linker-L^{A}-R^{T} and the other is —OH; and the remaining variables are as defined in the 2^{nd}, 3^{rd}, 4^{th}, 5^{th}, 6^{th}, 7^{th}, 8^{th} or 9^{th} embodiment of the fourth aspect or any embodiments described therein.

In a 11^{th} embodiment of the fourth aspect, for the conjugate of formula (III) or a pharmaceutically acceptable salt thereof, -L-PS is represented by formula (IVB):

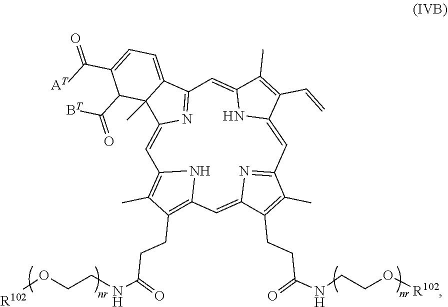

or a pharmaceutically acceptable salt thereof, wherein one of A^{T} and B^{T} is —Z-L^{P}-linker-L^{A}-R^{T} and the other is —OH or —OCH₃; R¹⁰² is H or Me; and nr is an integer from 2 to 8; and the remaining variables are as defined in the 2^{nd}, 3^{rd}, 4^{th}, 5^{th}, 6th, 7^{th}, 8^{th} or 9^{th} embodiment of the fourth aspect or any embodiments described therein.

In a 12^{th} embodiment of the fourth aspect, for the conjugate of formula (III) or a pharmaceutically acceptable salt thereof, -L-PS is represented by formula (IV), (IVA) or (IVB), Z in formula (IV), (IVA) or (IVB) is —NH—; and the remaining variables are as defined in the 2^{nd}, 3^{rd} 4^{th}, 5^{th}, 6^{th}, 7th, 8^{th}, 9^{th}, 10^{th} or 11^{th} embodiment of the fourth aspect or any embodiments described therein.

In a 13^{th} embodiment of the fourth aspect, for the conjugate of formula (III) or a pharmaceutically acceptable salt thereof, -L-PS is represented by formula (IV), (IVA) or (IVB), Z in formula (IV), (IVA) or (IVB) is —O—; and the remaining variables are as defined in the 2^{nd}, 3^{rd} 4^{th}, 5^{th}, 6^{th}, 7th, 8^{th}, 9^{th}, 10^{th} or 11^{th} embodiment of the fourth aspect or any embodiments described therein.

In a 14^{th} embodiment of the fourth aspect, for the conjugate of formula (III) or a pharmaceutically acceptable salt thereof, -L-PS is represented by formula (IV), (IVA) or (IVB), L^{P} in formula (IV), (IVA) or (IVB) is a non-cleavable spacer; and the remaining variables are as defined in the 2^{nd}, 3^{rd}, 4^{th}, 5^{th}, 6^{th}, 7^{th}, 8^{th}, 9^{th}, 10^{th}, 11^{th}, 12^{th} or 13^{th} embodiment of the fourth aspect or any embodiments described therein. In some embodiments, L^{P} comprises a hydrophilic moiety. In some embodiments, L^{P} comprises a PEG moiety.

In a 15^{th} embodiment of the fourth aspect, for the conjugate of formula (III) or a pharmaceutically acceptable salt thereof, -L-PS is represented by formula (IV), (IVA) or (IVB), L^{P} in formula (IV), (IVA) or (IVB) is *—CH₂CH₂—(OCH₂CH₂)ₘ—, wherein * is the sited connected to the Linker group, and m is an integer from 0 to 30; and the remaining variables are as defined in the 14^{th} embodiment. In some embodiments, m is an integer from 2 to 10. In some embodiments, m is an integer from 2 to 8. In some embodiments, m is an integer from 3 to 5. In some embodiments, m is 2, 3, 4, 5, 6, 7 or 8. In some embodiments, m is 6.

In a 16^{th} embodiment of the fourth aspect, for the conjugate of formula (III) or a pharmaceutically acceptable salt thereof, -L-PS is represented by formula (IV), (IVA) or (IVB), L^{P} in formula (IV), (IVA) or (IVB) is *—CH₂CH₂—(OCH₂CH₂)₄—, wherein * is the sited connected to the Linker group; and the remaining variables are as defined in the 14^{th} embodiment.

In a 17^{th} embodiment of the fourth aspect, for the conjugate of formula (III) or a pharmaceutically acceptable salt thereof, -L-PS is represented by formula (IV), (IVA) or (IVB), L^{P} in formula (IV), (IVA) or (IVB) is a cleavable group; and the remaining variables are as defined in the 2^{nd}, 3^{rd}, 4^{th}, 5^{th}, 6^{th}, 7^{th}, 8^{th}, 9^{th}, 10^{th}, 11^{th}, 12^{th} or 13^{th} embodiment of the fourth aspect or any embodiments described therein. In some embodiments, the cleavable space comprises an enzyme labile group, a pH labile group, a disulfide group, a ROS cleavable group, a photocleavable group. In some embodiments, the enzyme labile group is a group cleavable by an esterase, a peptidase, an aminopeptidase, a β-galactosidase, a β-glucuronidase, a carboxylesterase, a caspase, a diaphorase, a histone deacetylase, a legumain, or matrix metalloproteinase. In some embodiments, the enzyme labile group is a group cleavable by cathepsin B.

In a 18^{th} embodiment of the fourth aspect, for the conjugate of formula (III) or a pharmaceutically acceptable salt thereof, -L-PS is represented by formula (IV), (IVA) or (IVB), L^{P} in formula (IV), (IVA) or (IVB) is comprises a peptide and a self-immolative group; and the remaining variables are as defined in the 2^{nd}, 3^{rd} 4^{th}, 5^{th}, 6^{th}, 7^{th}, 8^{th}, 9^{th}, 10^{th}, 11^{th}, 12^{th} or 13^{th} embodiment of the fourth aspect or any embodiments described therein.

In a 19^{th} embodiment of the fourth aspect, for the conjugate of formula (III) or a pharmaceutically acceptable salt thereof, -L-PS is represented by formula (IV), (IVA) or (IVB), L^{P} in formula (IV), (IVA) or (IVB) is represented by the following formula:

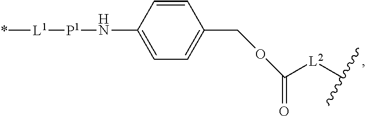

wherein:
*- represents a bond to the group represented by Linker;

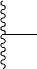

represents a bond to the group Z;
L¹ and L² are each independently a connecting spacer;
P¹ is a peptide comprising 2 to 5 amino acid residues; and the remaining variables are as defined in the 2^{nd}, 3^{rd}, 4^{th}, 5th, 6^{th}, 7^{th}, 8^{th}, 9^{th}, 10^{th}, 11^{th}, 12^{th} or 13^{th} embodiment of the fourth aspect or any embodiments described therein.

In a 20^{th} embodiment of the fourth aspect, for the conjugate or a pharmaceutically acceptable salt thereof described in the 18^{th} embodiment, P¹ is a peptide selected from Phe-Arg-Arg-Gly, Glu-Val-Cit, Val-Cit, Cit-Val, Gly-Gly-Phe, Val-Ala, or Ala-Val; and the remaining variables are as defined in the 19^{th} embodiment of the fourth aspect or any embodiments described therein.

In a 21^{st} embodiment of the fourth aspect, for the conjugate or a pharmaceutically acceptable salt thereof described in the 19^{th} embodiment, P¹ is:

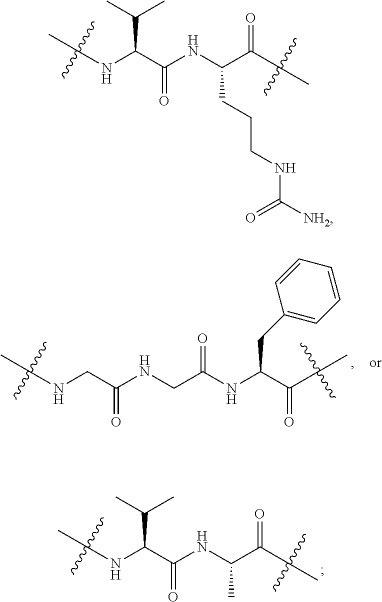

and the remaining variables are as defined in the 18^{th} embodiment of the fourth aspect or any embodiments described therein.

In a 22^{nd} embodiment of the fourth aspect, for the conjugate or a pharmaceutically acceptable salt thereof described in the 19^{th} embodiment, L¹ comprises a hydrophilic moiety; and the remanding variables are as defined in the 19^{th}, 20^{th} or 21^{st} embodiment of the fourth aspect. In some embodiment, L¹ comprises a PEG moiety.

In a 23^{rd} embodiment of the fourth aspect, for the conjugate or a pharmaceutically acceptable salt thereof described in the 19^{th} embodiment, L¹ is *—CH₂—CH₂—(OCH₂CH₂)—C(═O)—**; n is an integer from 0 to 30; *- represents a bond to the Linker group; **-represents a bond to P¹; and the remainding variables are as defined in the 19^{th}, 20^{th} or 21^{st} embodiment of the fourth aspect or any embodiments described therein. In some embodiments, n is an integer from 2 to 16. In some embodiments, n is an integer from 2 to 10. In some embodiment, n is an integer from 2 to 8. In some embodiments, n is an integer from 3 to 5. In some embodiments, n is 2, 3, 4, 5, 6, 7 or 8.

In a 24^{th} embodiment of the fourth aspect, for the conjugate or a pharmaceutically acceptable salt thereof described in the 19^{th} embodiment, L¹ is *—CH₂—CH₂—(OCH₂CH₂)₃—C(═O)—**, wherein *- represents a bond to the Linker group; and **- represents a bond to P¹; and the remainding variables are as defined in the 19^{th}, 20^{th} or 21^{st} embodiment of the fourth aspect or any embodiments described therein.

In a 25^{th} embodiment of the fourth aspect, for the conjugate or a pharmaceutically acceptable salt thereof described in the 19^{th} embodiment, L² is —NH—C₁₋₆alkyl-**, wherein ** is the site connected to Z; and the remainding variables areas defined in the 19^{th}, 20^{th}, 21^{st}, 22^{nd}, 23^{rd} or 24^{th} embodiment of the fourth aspect or any embodiments described therein.

In a 26^{th} embodiment of the fourth aspect, for the conjugate or a pharmaceutically acceptable salt thereof described in the 18^{th} embodiment, L² is —NH—CH₂CH₂—**, wherein ** is the site connected to Z; and the remainding variables areas defined in the 19^{th}, 20^{th}, 21^{st}, 22^{nd}, 23^{rd} or 24^{th} embodiment of the fourth aspect or any embodiments described therein.

In a 27^{th} embodiment of the fourth aspect, the conjugate of the present disclosure is represented by the following formula:

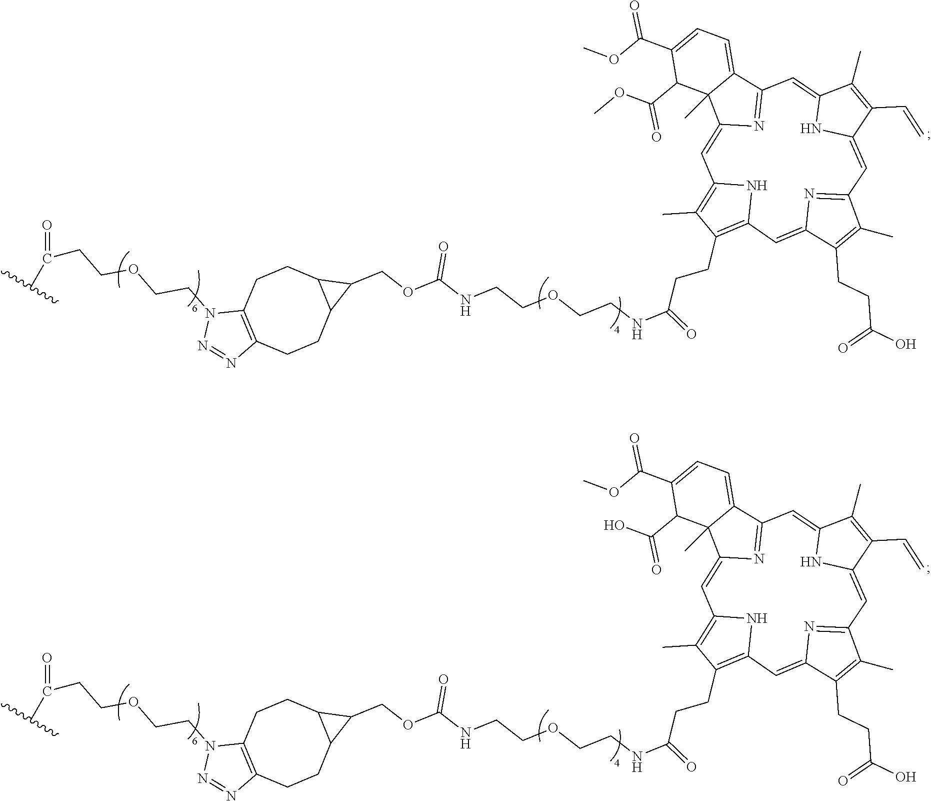

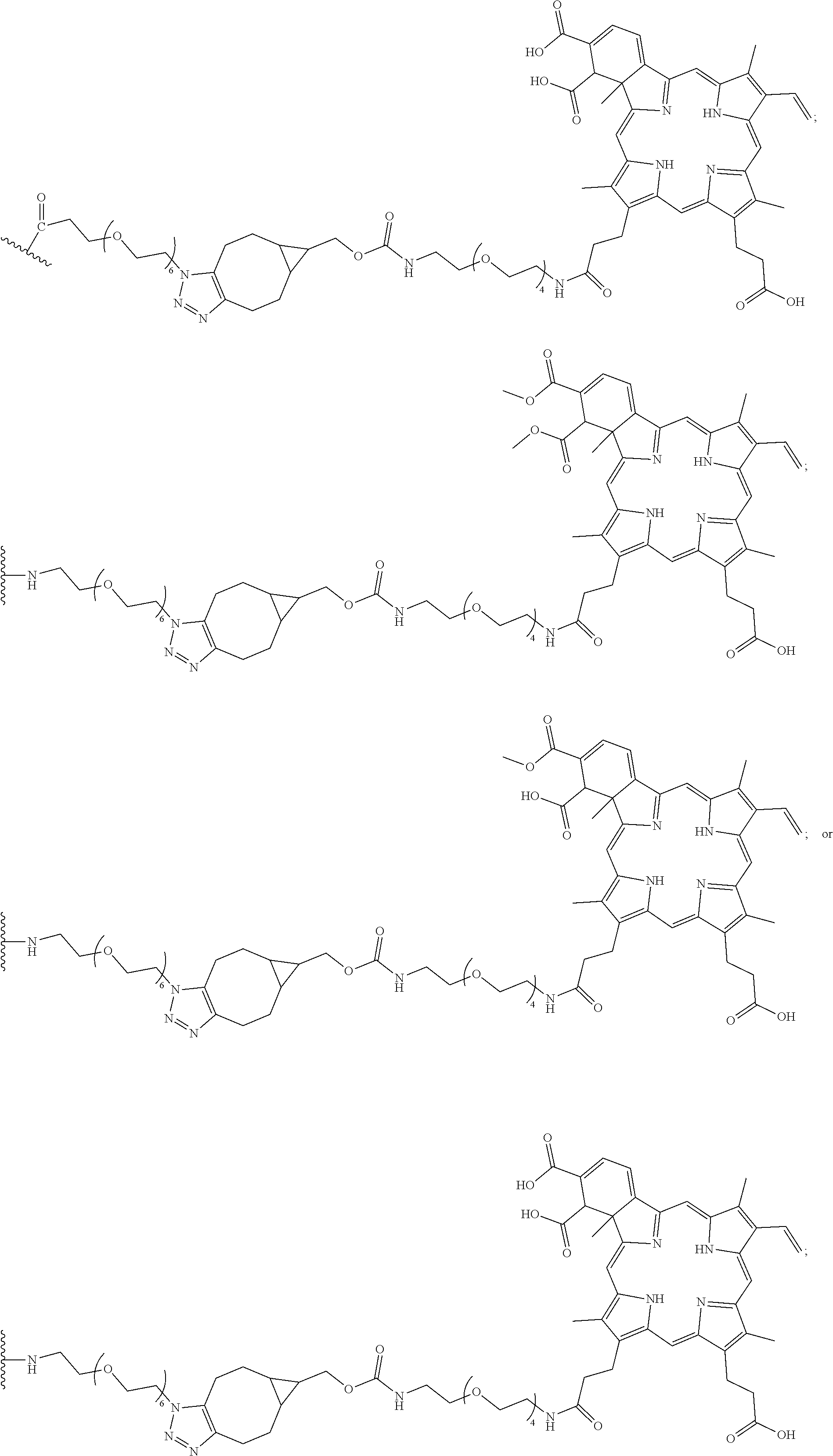

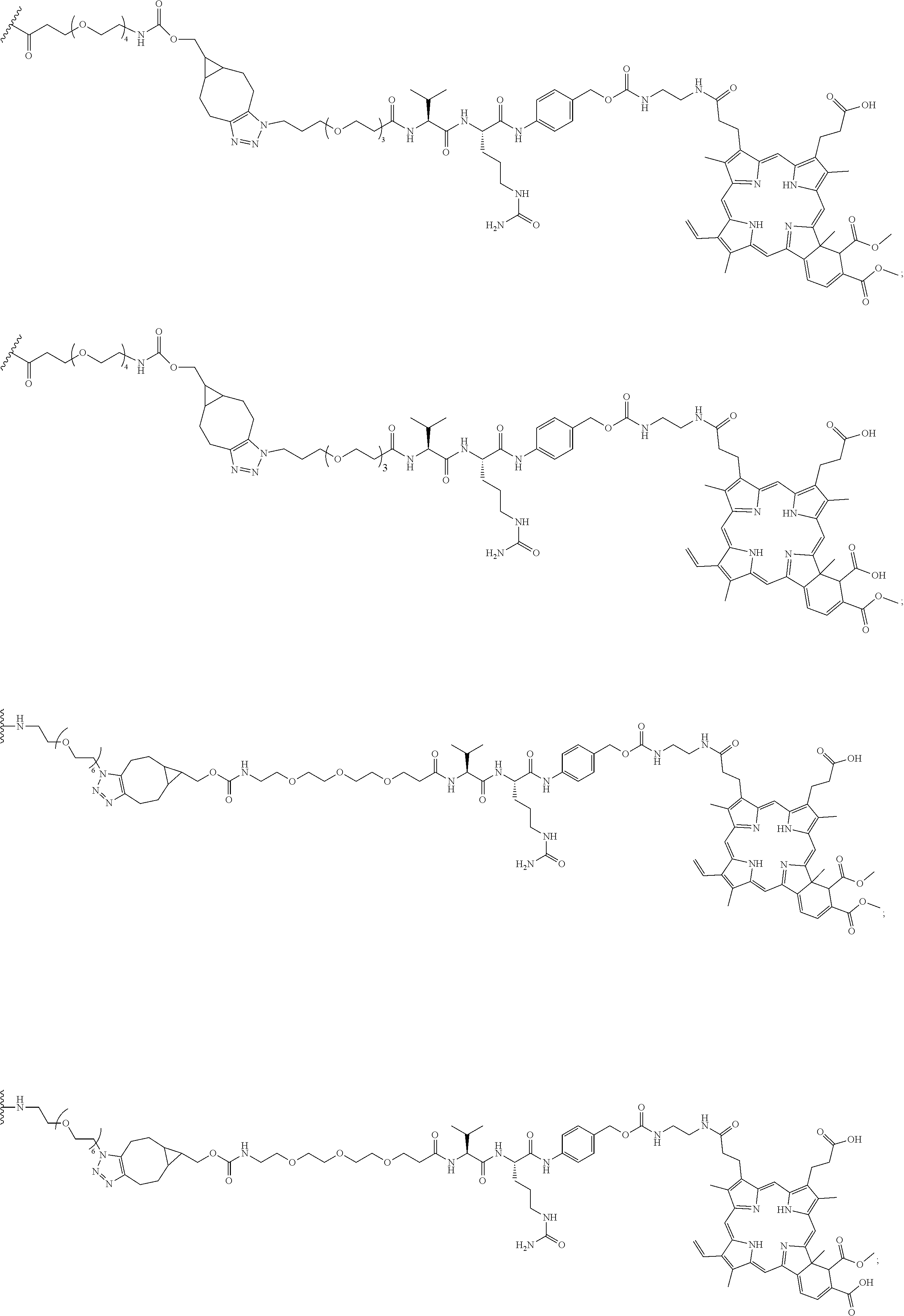

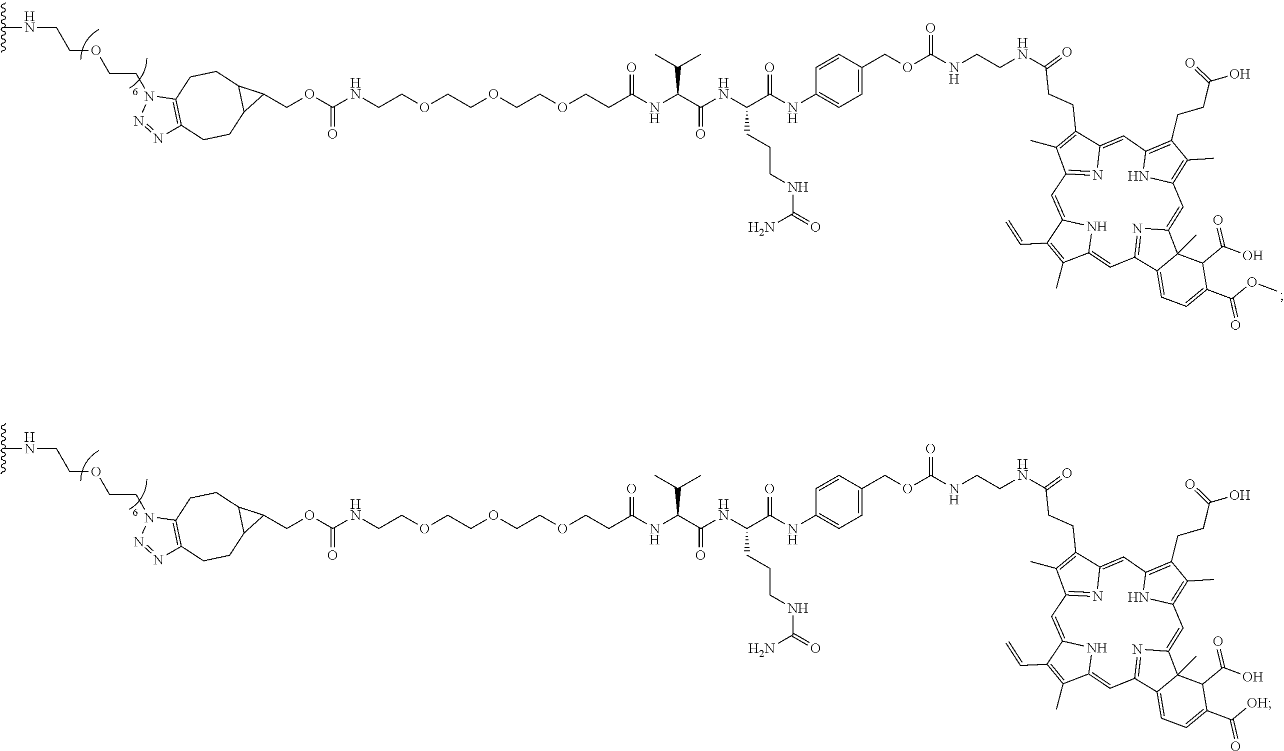

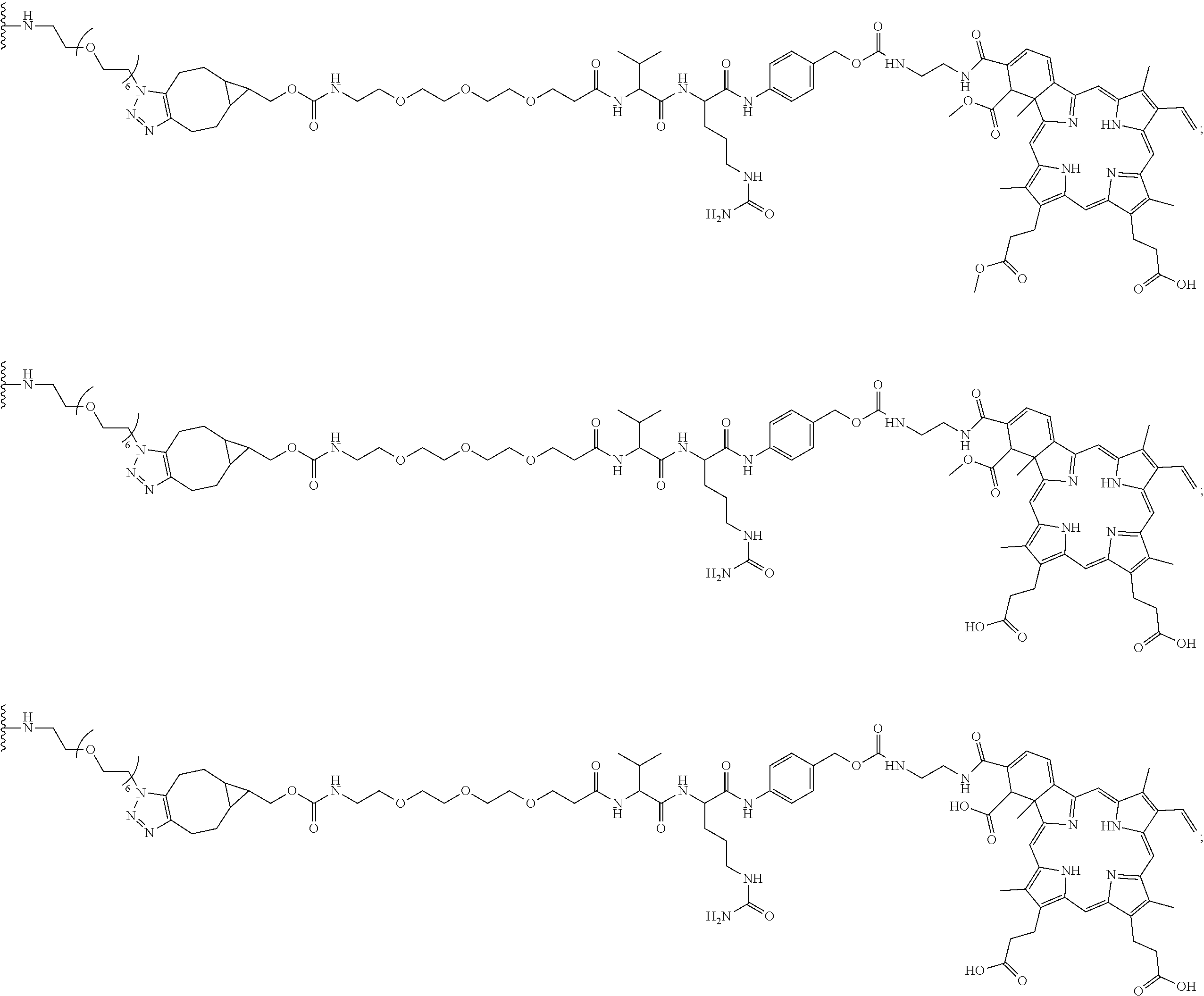

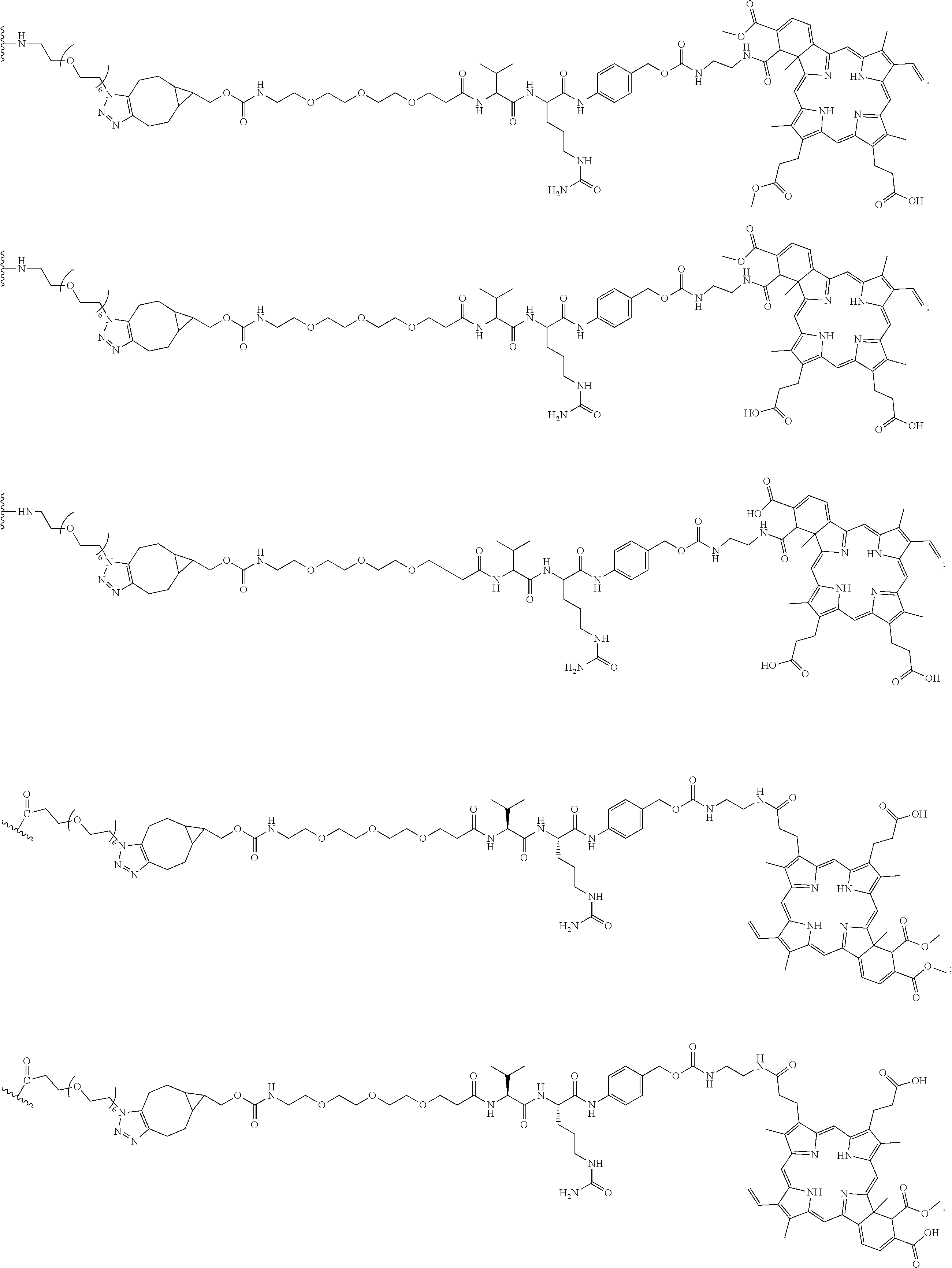

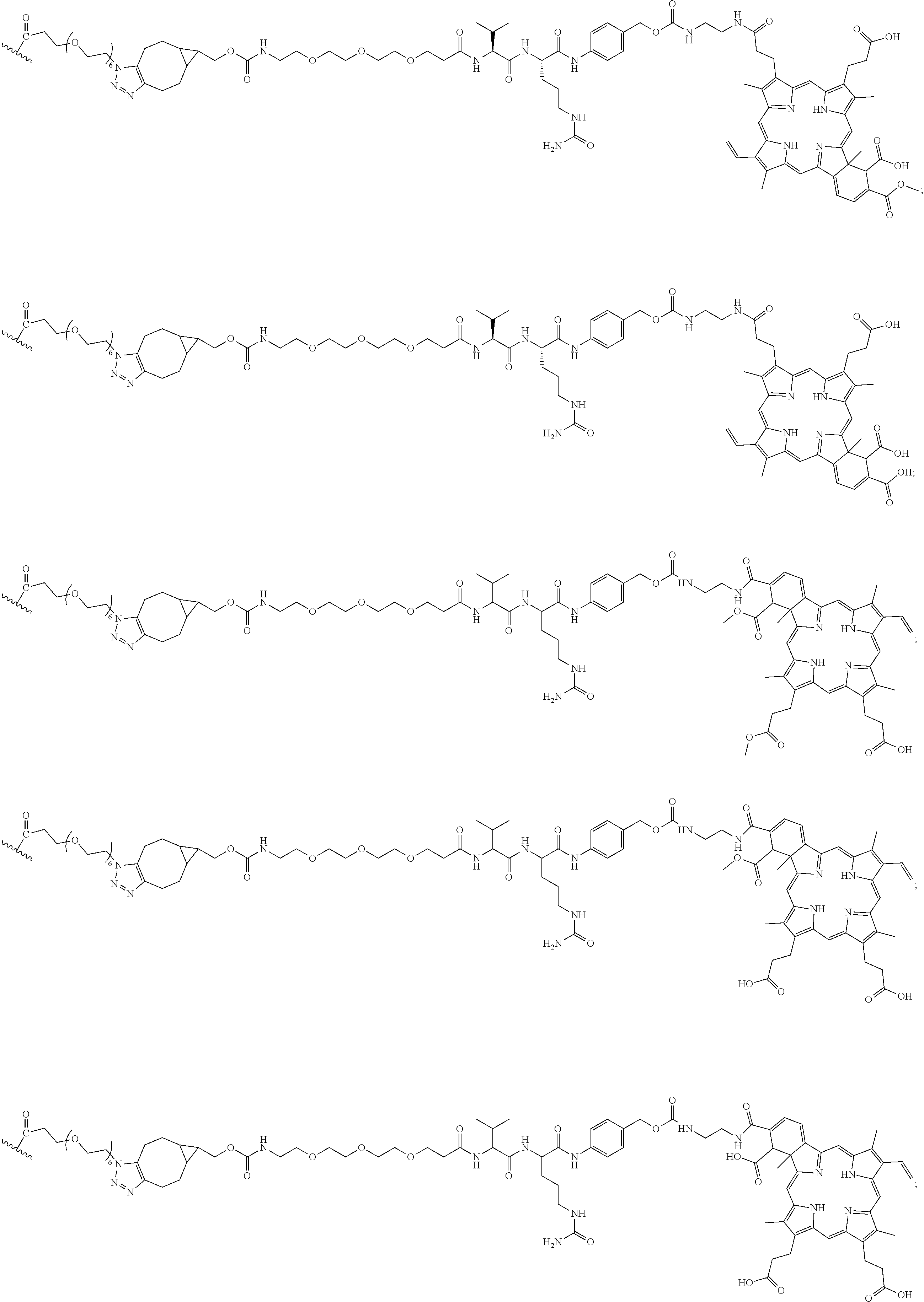

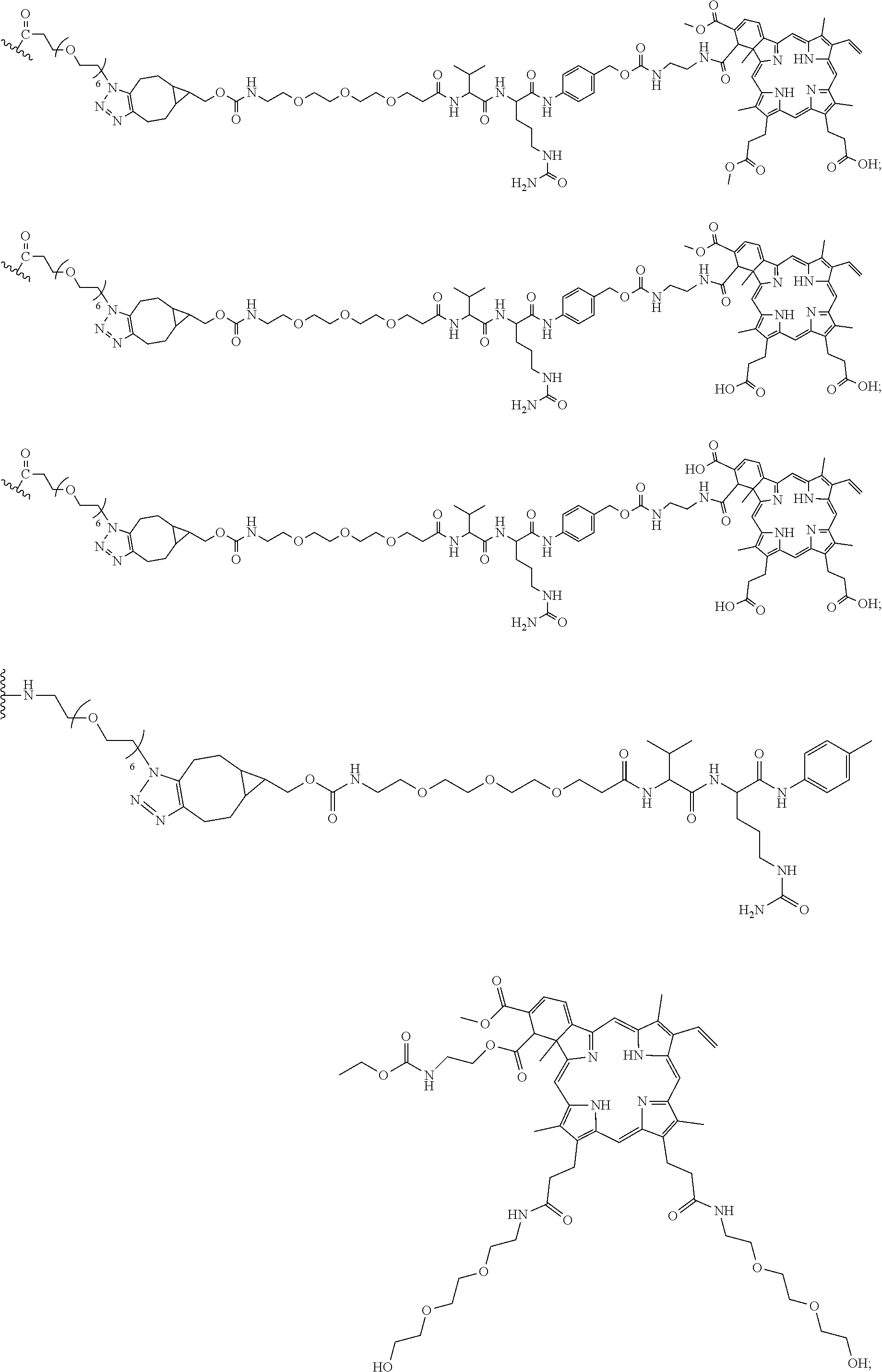

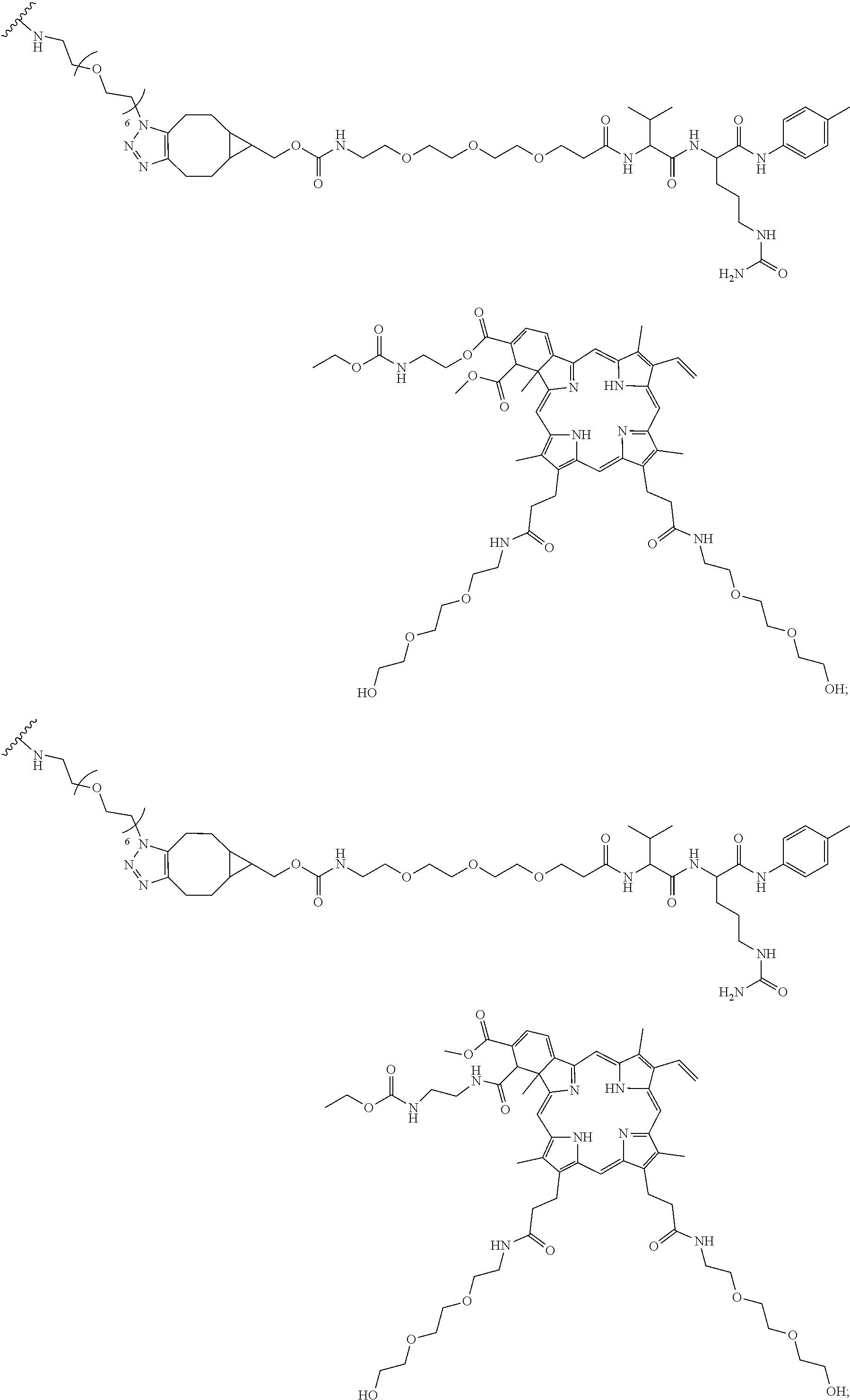

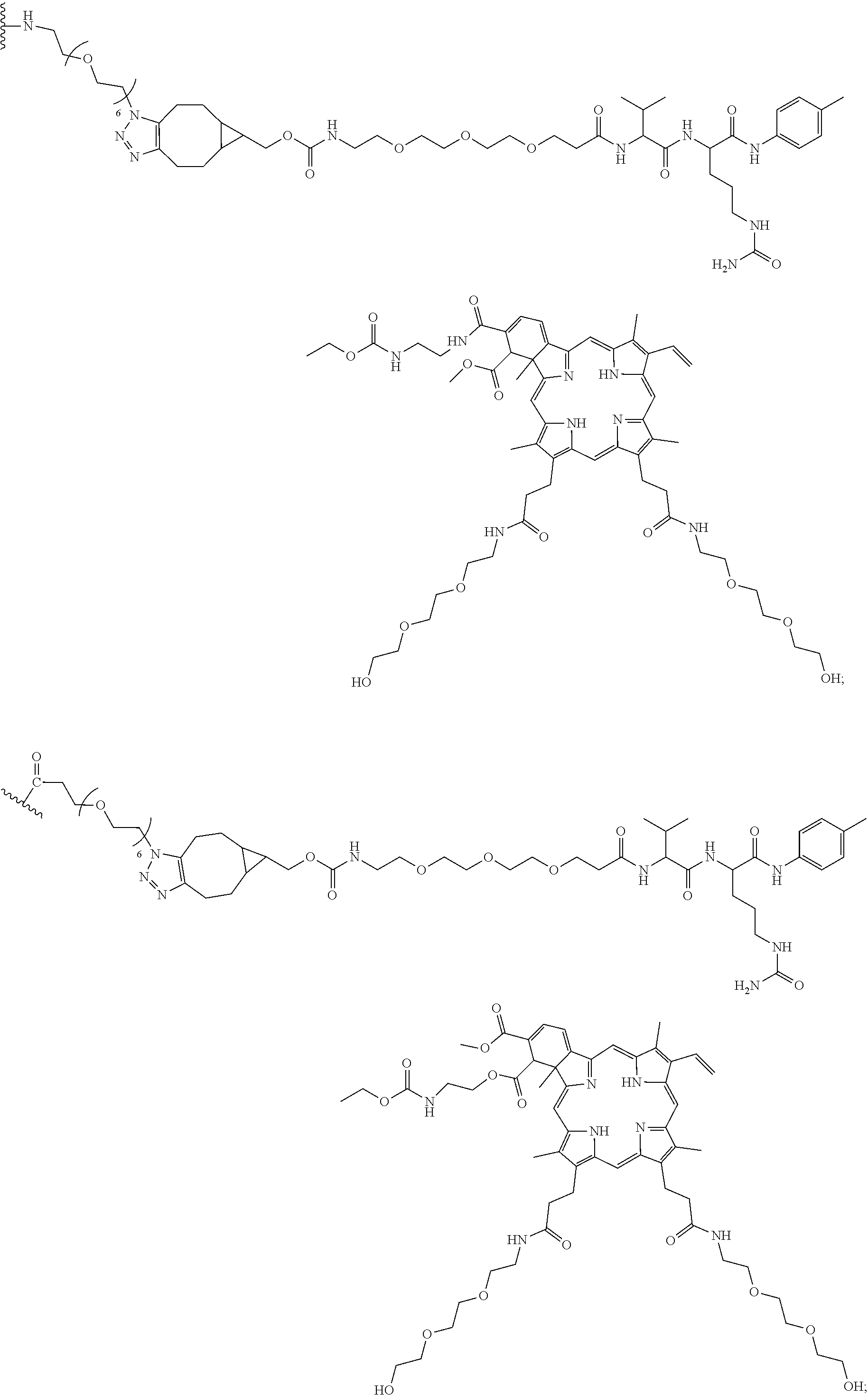

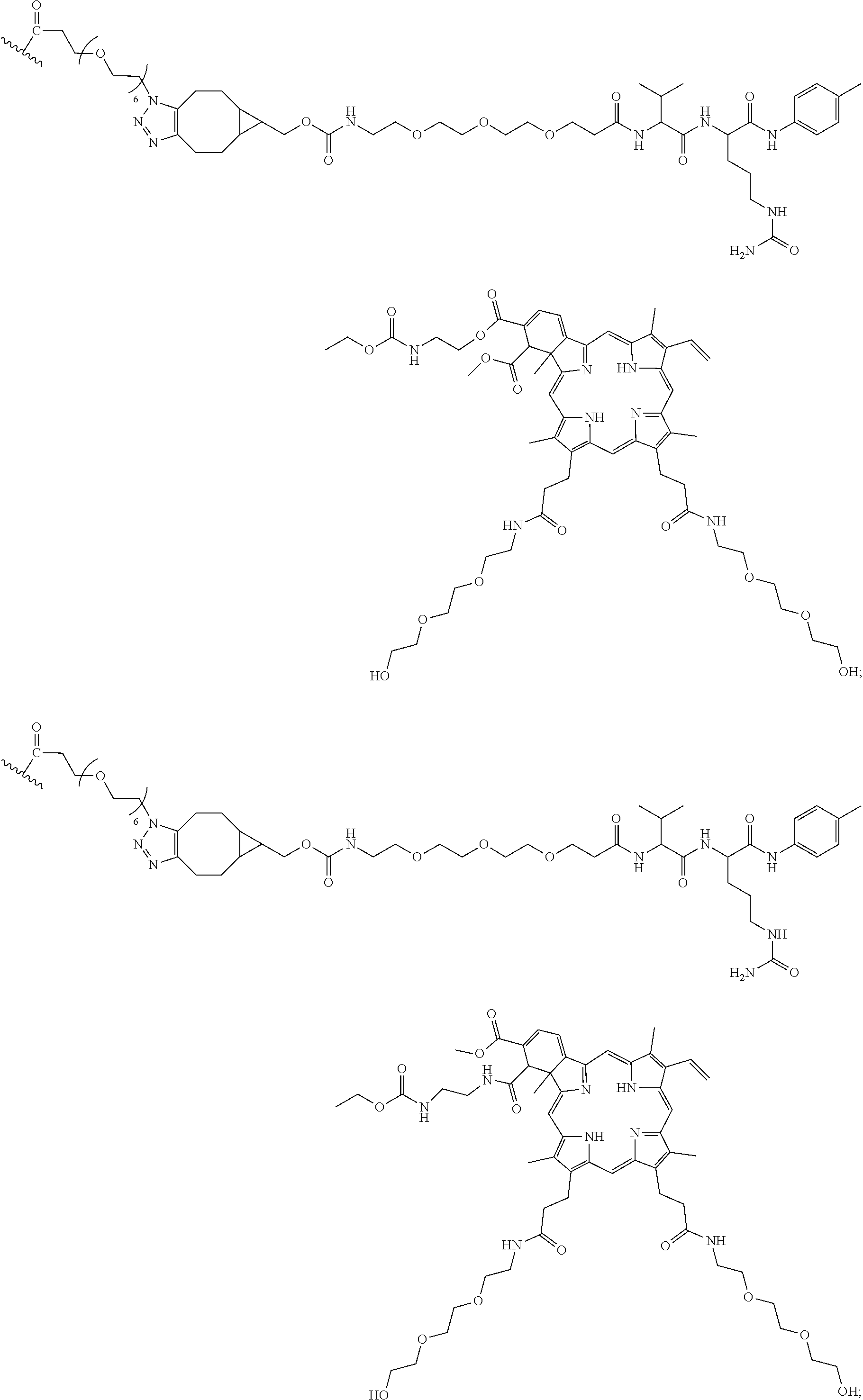

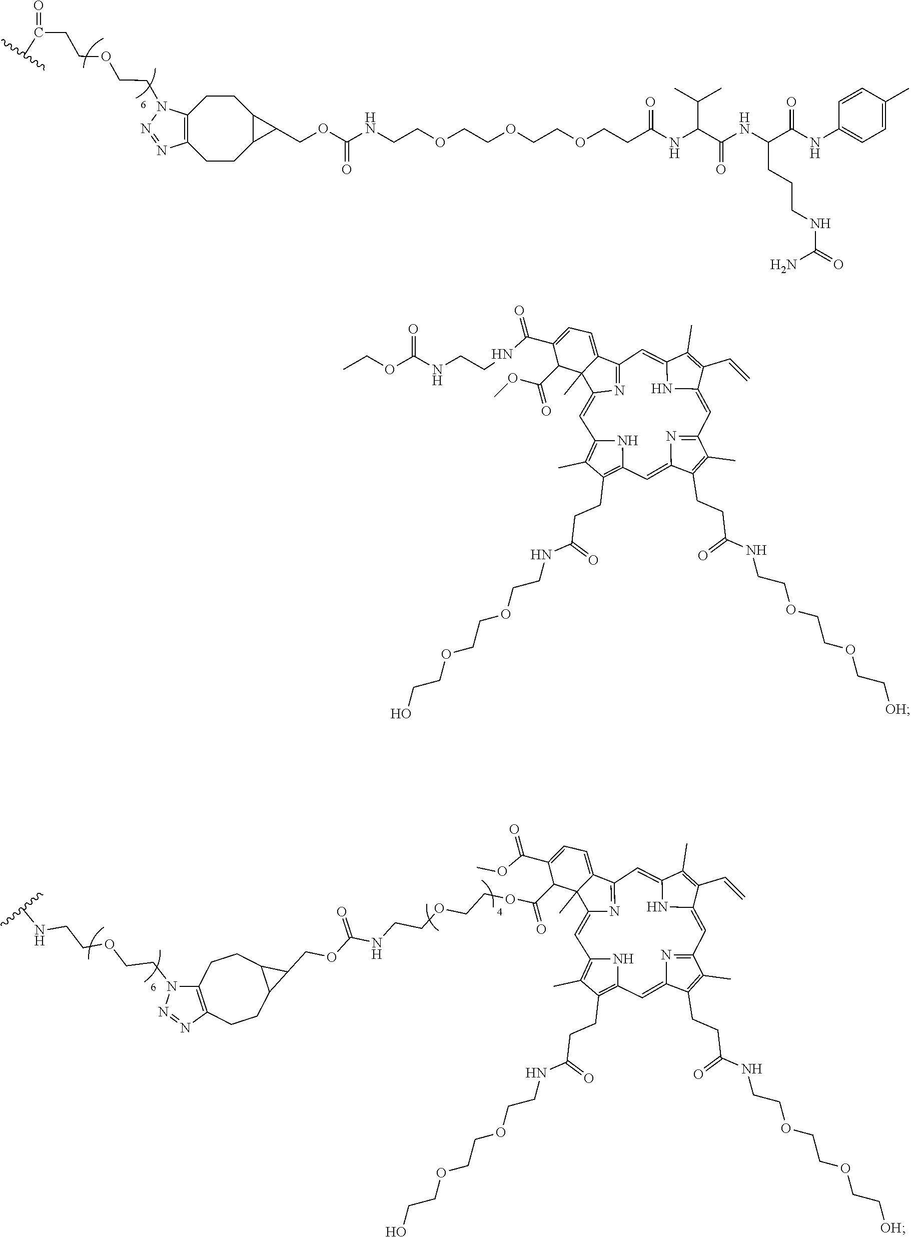

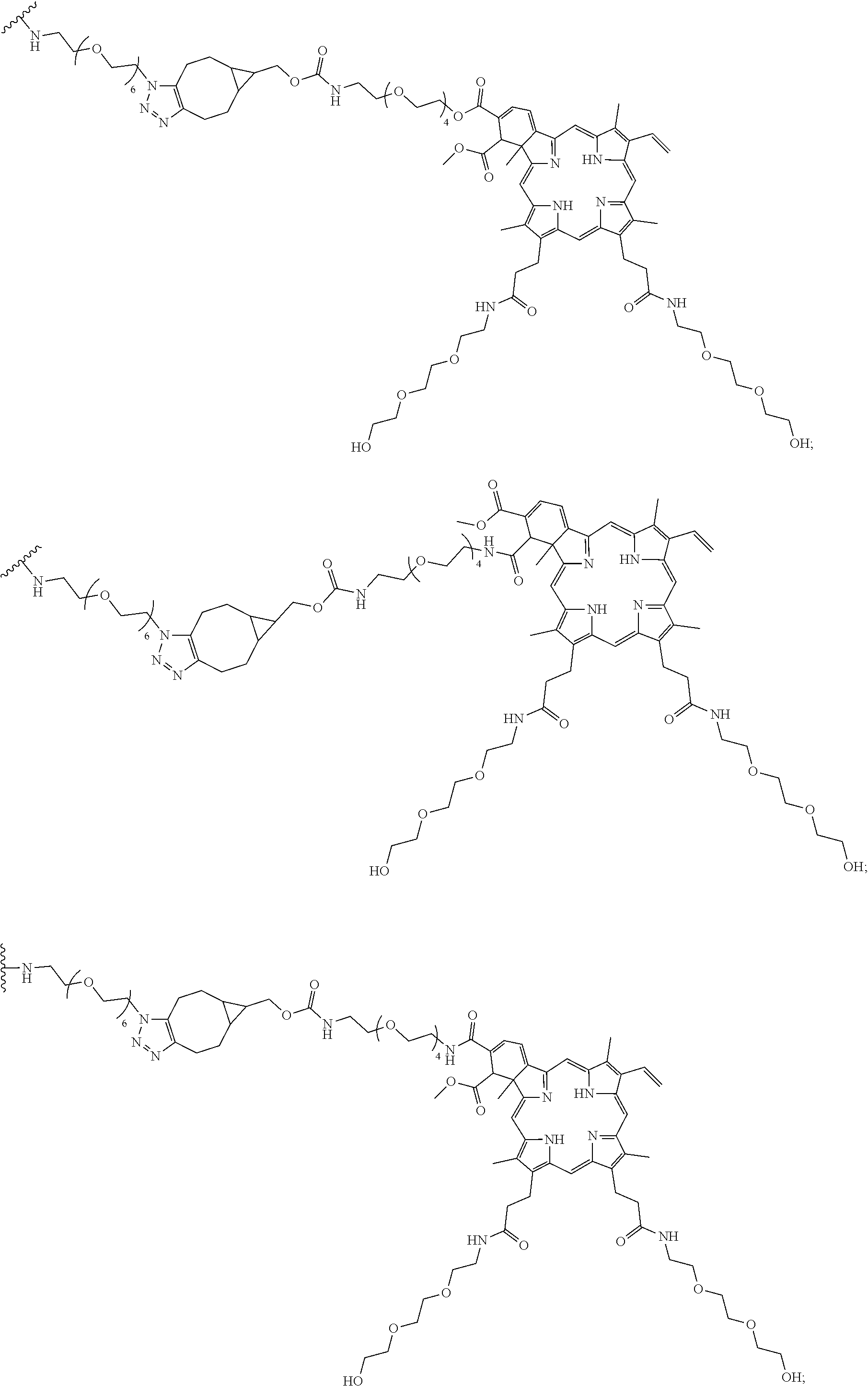

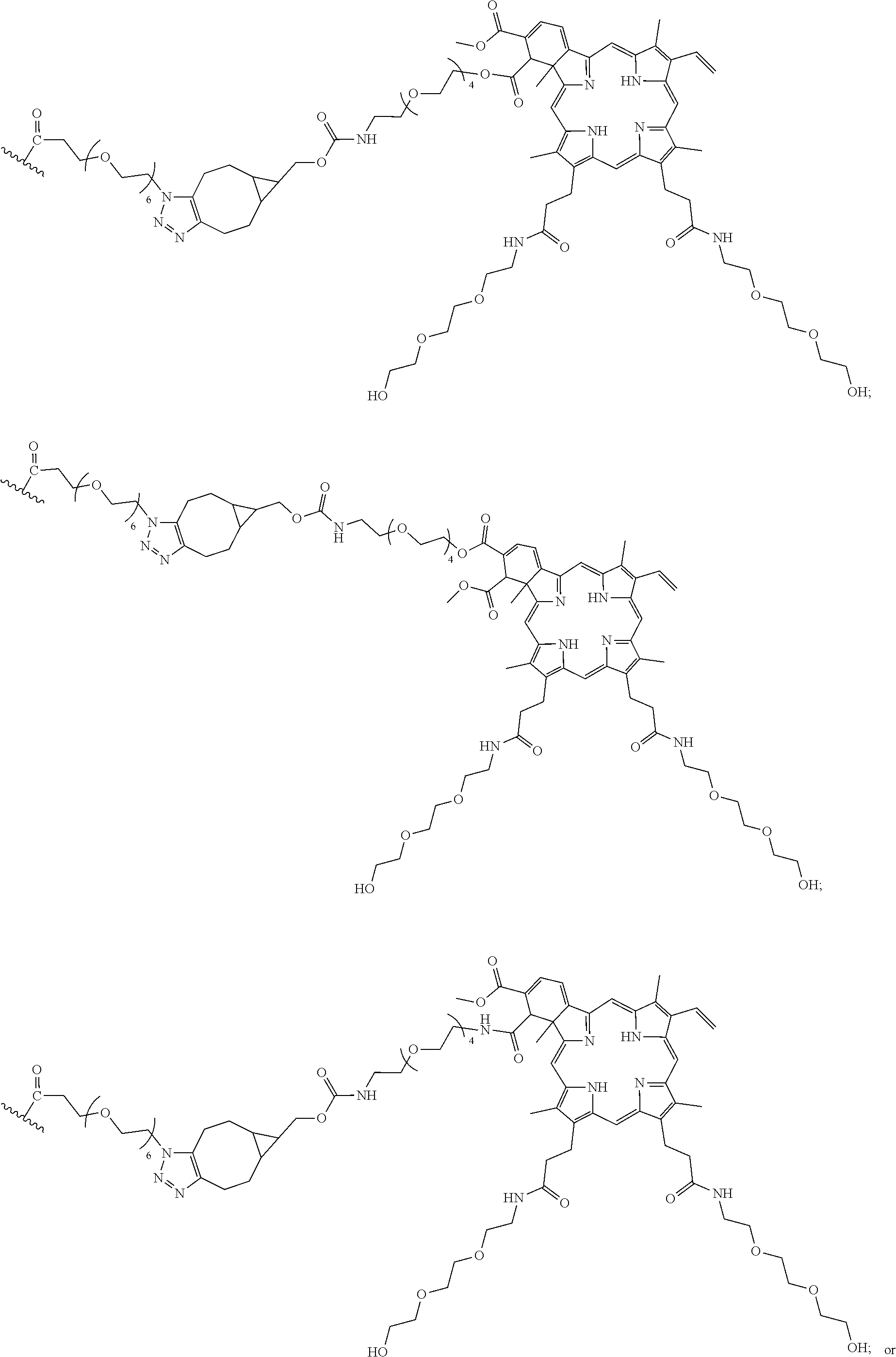

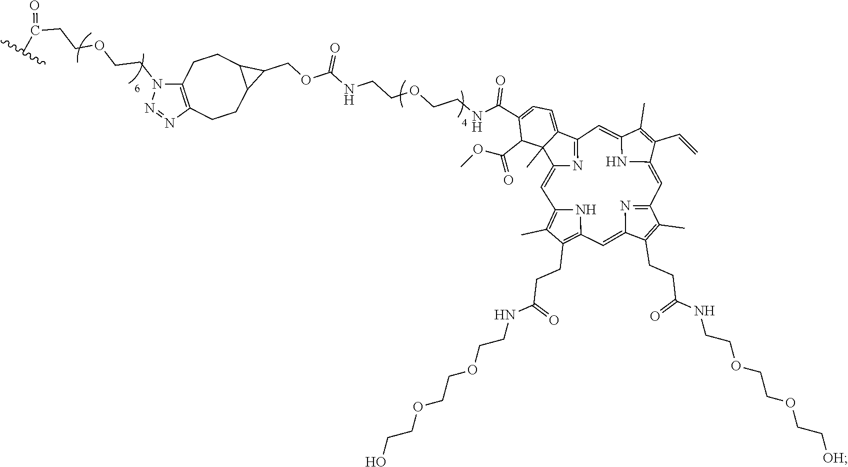

or a pharmaceutically acceptable salt thereof, wherein

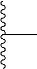

indicates a bond to the targeting agent. Alternatively, as part of the 27^{th} embodiment of the fourth aspect, the conjugate of the present disclosure is represented by the following formula:

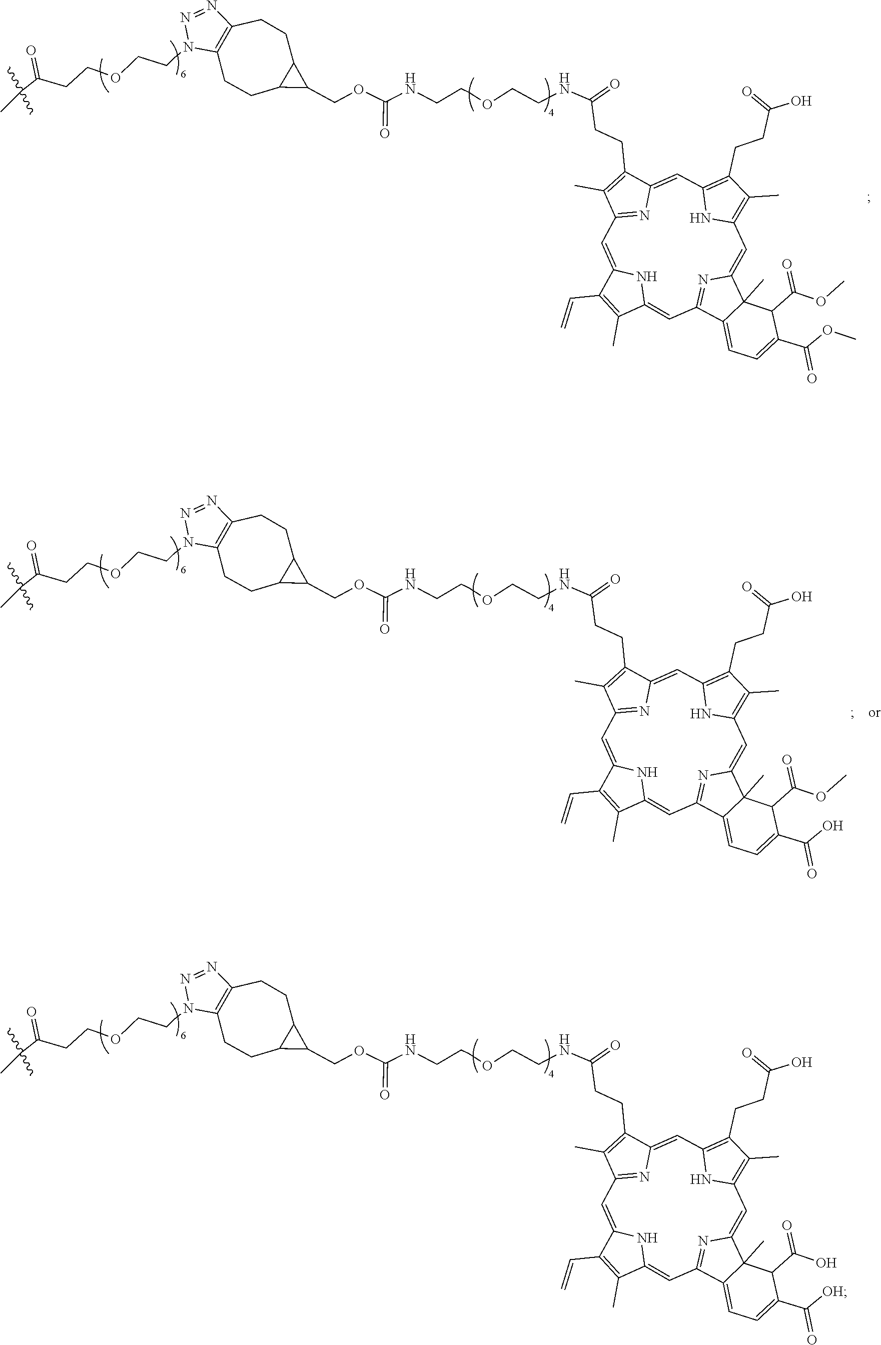

or a pharmaceutically acceptable salt thereof, wherein

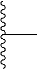

indicates a bond to the targeting agent.

### Targeting Agents

In some embodiment, for the modified targeting agent described in the third aspect (e.g., the 1^{st} to 6^{th} embodiments of the third aspect or any embodiments described therein) or the conjugate described in the fourth aspect (e.g., the 1^{st} to 27^{th} embodiments of the fourth aspect or any embodiments described therein), the targeting agent can be a small molecule (e.g., folic acid), a protein, a peptide (e.g., cell penetrating peptides), hormone, hapten, avidin, strepavidin, biotin, carbohydrate, oligosaccharide, polysaccharide, nucleic acid, fragment of DNA, fragment of RNA, aptamer, or nanocarrier (e.g., liposome, gold nanoparticle, polymeric nanoparticle, virus-like particles) that binds to the surface of a target cell.

In some embodiments, the targeting agent is an antibody or an antigen-binding fragment thereof.

In some embodiments, the targeting agent is an antibody or an antigen-binding fragment thereof that binds to an adipose cell, blood cell, cancer cell, endothelial cell, epithelial cell, immune cell, neuron, skin cell, stem cell, tumor cell, myeloid derived suppressor cell. In some embodiments, the antibody or an antigen-binding fragment thereof binds to a cancer-associated fibroblast, a tumor-associated macrophage, a T cell, or a regulatory T cell.

In some embodiments, the targeting agent is an antibody or an antigen-binding fragment thereof that binds to EGFR/HER1, HER2, HER3, HER4, VEGF, VEGFR, VEGFR2, EpCAM, E-Cad, Folate Receptor alpha, Fibroblast activation protein (FAP), CD1c, CD2, CD3, CD4, CD5, CD6, CD7, CD8, CD11a, CD11b, CD11c, CD14, CD16, CD18, CD19, CD20, CD21, CD23, CD25, CD26, CD27, CD28, CD30, CD31, CD32, CD33, CD44, CD45, CD52, CD56, CD62L, CD64, CD66b, CD69, CD80, CD86, CD90, CD103, CD122, CD123, CD127, CD163, CD206, CD235a, CXCR6, MHC-II, CCR4, CCR5, CCR7, CLA, PD-1, PD-L1 or CTLA-4, CEA, MUC-1, PSMA, Cancer antigen 125 (CA125), Alpha-fetoprotein (AFP), Lewis Y antigen, TAG-72, IL-13R, melanoma-associated antigen (MAGE)₁, MAGE2, MAGE3, MAGE4, tumor-associated glycoprotein 72 (TAG-72), gp100, p97 melanoma antigen, human milk fat globule (HMFG), melanoma antigen recognized by T cells 1 (MART1), B melanoma antigen (BAGE) 1, BAGE2, G antigen (GAGE) 1, GAGE2, GAGE3, GAGE4, GAGE5, GAGE6, breast cancer-associated DF3 antigen, New York esophageal squamous cell carcinoma 1 (NY-ESO-1), mesothelin, GITR, OX40, FR4, CXCR4, CCL4, Gr-1, IL-4Ra, IL-1Ra, CXCR2, or LAG-3.

In some embodiments, the targeting agent is an antibody or an antigen-binding fragment thereof that binds to CD2 or EGFR.

In some embodiments, the targeting agent (e.g., an antibody or an antigen-binding fragment thereof) described above comprises a reactive group that can form a covalent bond with R^{T}. In some embodiments, the reactive group is selected from an amine group, a thiol group or an amide group. In some embodiments, the targeting agent (e.g., an antibody or an antigen-binding fragment thereof) described above comprises a lysine, a cysteine or a glutamine residue that can form a covalent bond with the R^{T} group. In some embodiments, the lysine, cysteine or glutamine residue is connected to the R^{T} group through the side chain, i.e., amine group of the lysine residue, amide group of the glutamine residue or thiol group of the cysteine residue. In some embodiments, the targeting agent (e.g., an antibody or an antigen-binding fragment thereof) can be covalently bonded to the R^{T} group through an amine group of the terminal amino acid.

In some embodiments, the targeting agent the targeting agent (e.g., an antibody or an antigen-binding fragment thereof) described above comprises the following group:

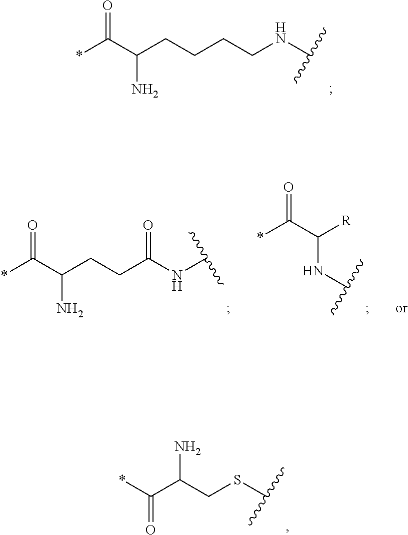

wherein R is side chain of an amino acid residue; *- represents a bond to the remaining targeting agent and

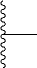

represents a bond to R^{T}.

### Methods of Making Photosensitizer-Targeting Agent Conjugates

The conjugates of the present disclosure (e.g., the conjugate described in the fourth aspect, the 1^{st} to 27^{th} embodiments of the fourth aspect or any embodiments described therein) can be prepared by any suitable methods known in the art.

In some embodiments, the targeting agent (e.g., an antibody or an antigen-binding fragment thereof) can be conjugated with a linker-photosensitizer compound described herein (e.g., those described in the second aspect, the 1^{st} to 26^{th} embodiments of the second aspect or any embodiments described therein) via a reactive group located on the targeting agent. In some embodiments, the reactive group is amine group of a lysine residue, amide group of a glutamine residue, thiol group of a cysteine residue or an amine group of a terminal amino acid residue. Either chemical conjugation or enzymatic conjugation reaction can be used.

In some embodiments, the conjugates of the present disclosure can be prepared by reacting a modified targeting agent (e.g., those described in the third aspect, the 1^{st} to 6^{th} embodiments of the third aspect or any embodiments described therein) having a first reactive moiety with a photosensitizer compound (e.g., those described in the first aspect, the 1^{st} to 21^{st} embodiments of the first aspect or any embodiments described therein) having a second reactive moiety.

### Other Compounds of the Present Disclosure

In a fifth aspect, the present disclosure provide compounds that can be used for preparing a photosensitizer compound described in the first aspect. Also provided in the fifth aspect of the present disclosure is metabolite compound of the conjugates described in the fourth aspect. In a 1^{st} embodiment of the fifth aspect, the compound is represented by formula (VI):

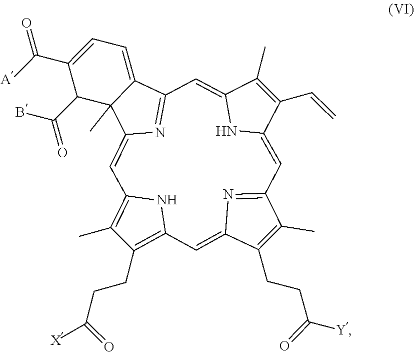

or a pharmaceutically acceptable salt thereof, wherein:
A′, B′, X′ and Y′ are each independently —OH, —OC₁₋₄alkyl, —N(R¹⁰⁰)(R¹⁰¹), or —Z—C₁₋₆allkylene-NH₂, provided one of A′, B′, X′ and Y′ is —Z—C₁₋₆allkylene-NH₂;
R¹⁰⁰ is H or C₁₋₃alkyl;
R¹⁰¹ is H, C₁₋₃alkyl or —(CH₂—CH₂—O)ₙᵣR¹⁰²;
R¹⁰² is H or Me;
nr is an integer from 1 to 16;
Z is —O— or —NR¹—; and
R¹ is H or C₁₋₃alkyl.

In a 2^{nd} embodiment of the fifth aspect, for the compound of formula (VI) or a pharmaceutically acceptable salt thereof, A′, B′, X′ and Y′ are defined as follows:
a) A′ and B′ are —OCH₃; and one of X′ and Y′ is —OCH₃ and the other one of X′ and Y′ is —Z—C₁₋₆allkylene-NH₂;
b) A′ and B′ are —OCH₃; and one of X′ and Y′ is —OH and the other one of X′ and Y′ is —Z—C₁₋₆allkylene-NH₂
c) one of A′ and B′ is —OCH₃, and the other one of A′ and B′ is —OH; and one of X′ and Y′ is —OH and the other one of X′ and Y′ is —Z—C₁₋₆allkylene-NH₂;
d) A′ and B′ are —OH; and one of X′ and Y′ is —OH and the other one of X′ and Y′ is —Z—C₁₋₆allkylene-NH₂; or
e) one of A′ and B′ is —OCH₃ and the other one of A′ and B′ is —Z—C₁₋₆allkylene-NH₂; and X′ and Y′ are both —NH(CH₂—CH₂—O)ₙᵣR¹⁰², wherein R¹⁰² is H or Me; and nr is an integer from 2 to 8; and the remaining variables are as defined in the 1st embodiment of the fifth aspect.

In a 2^{nd} embodiment of the fifth aspect, for the compound of formula (VI) or a pharmaceutically acceptable salt thereof, the compound is represented by formula (VIA):

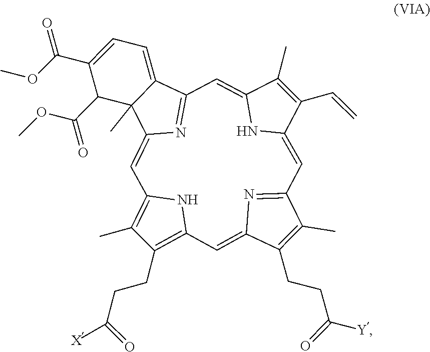

or a pharmaceutically acceptable salt thereof, wherein one of X′ and Y′ is —Z—C₁₋₆allkylene-NH₂ and the other is —OH; and the remaining variables are as defined in the 1^{st} embodiment of the fifth aspect.

In a 3^{rd} embodiment of the fifth aspect, for the compound of formula (VI) or a pharmaceutically acceptable salt thereof, the compound is represented by formula (VIB):

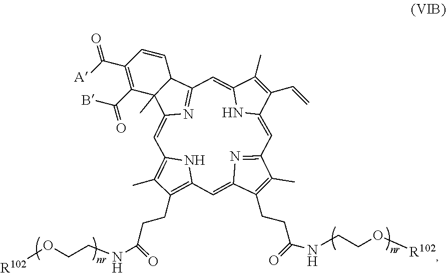

or a pharmaceutically acceptable salt thereof, wherein one of A′ and B′ is —Z—C₁₋₆allkylene-NH₂ and the other is —OH or —OCH₃; and the remaining variables are as defined in the 1st embodiment of the fifth aspect.

In a 4^{th} embodiment of the fifth aspect, for the compound of formula (VI), (VIA), or (VIB) or a pharmaceutically acceptable salt thereof, Z is —NH—; and the remaining variables are as defined in the 1^{st}, 2^{nd} or 3^{rd} embodiment of the fifth aspect.

In a 5^{th} embodiment of the fifth aspect, for the compound of formula (VI), (VIA), or (VIB) or a pharmaceutically acceptable salt thereof, Z is —O—; and the remaining variables are as defined in the 1^{st}, 2^{nd} or 3^{rd} embodiment of the fifth aspect.

In a 6^{th} embodiment of the fifth aspect, the compound is represented by the following formula:

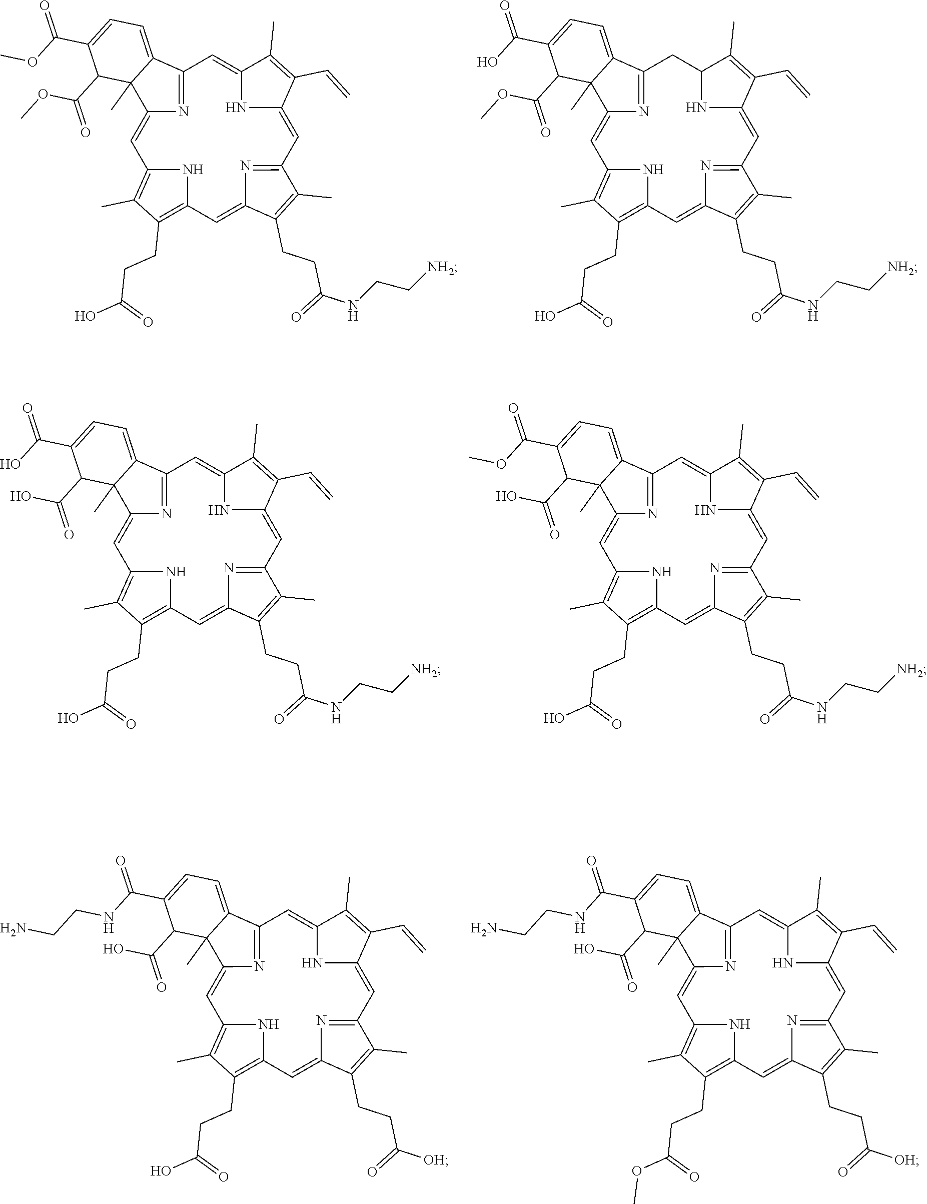

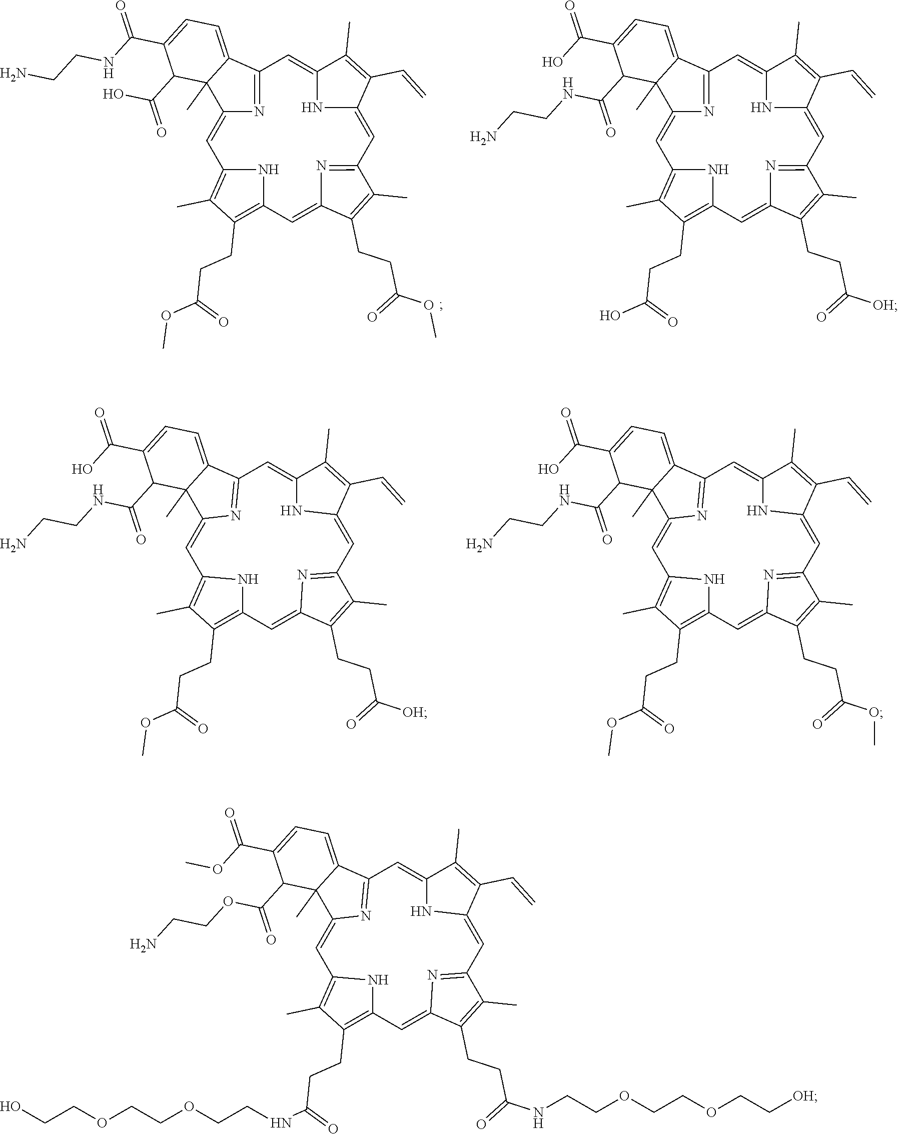

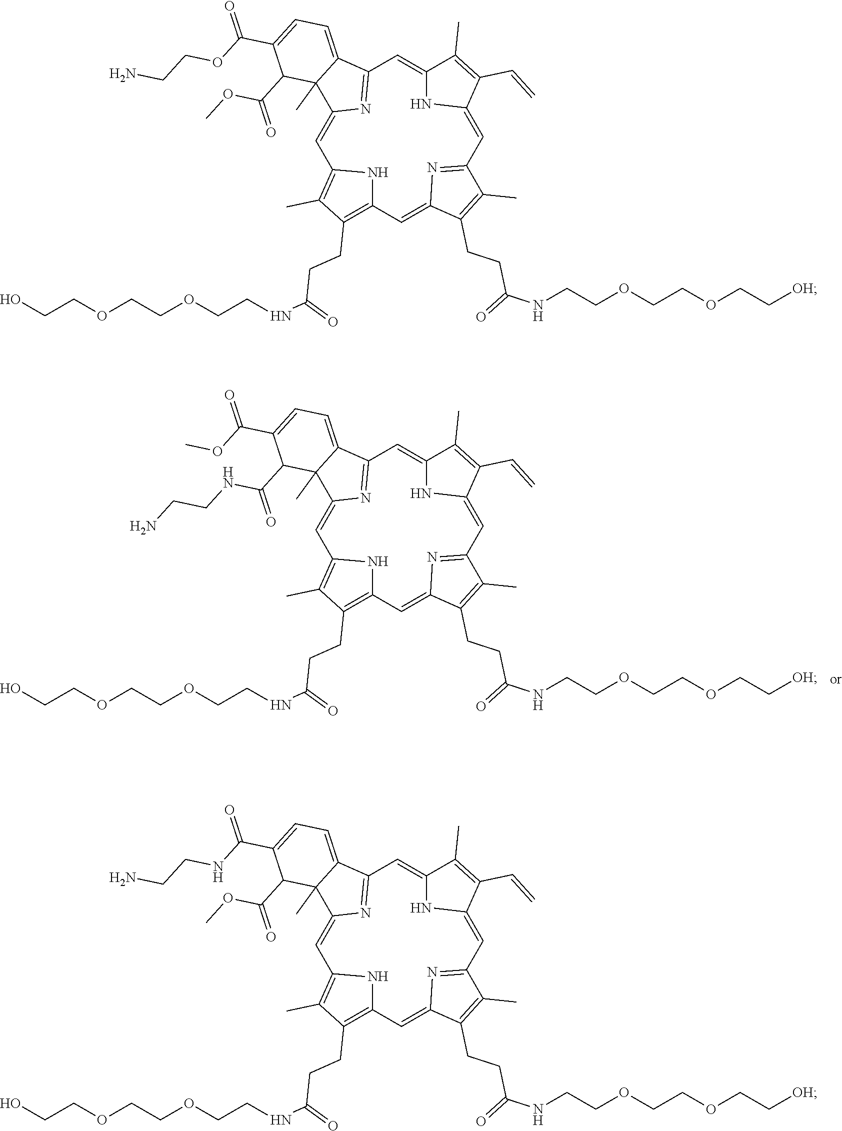 [SMILES: C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)OCCN)C1C(=O)OC ; C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)OC)C1C(=O)NCCN ; C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)NCCN)C1C(=O)OC]

or a pharmaceutically acceptable salt thereof, a regioisomer, or a pharmaceutically acceptable salt of the regioisomer. Alternatively, as part of the 6^{th} embodiment of the fifth aspect, the compound is represented by the following formula:

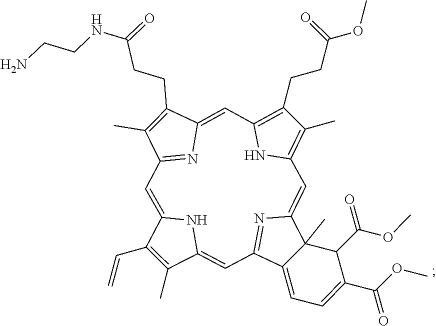 [SMILES: C=Cc1c(C)c2cc3nc(cc4[nH]c(cc5nc(cc1[nH]2)C(C)=C5CCC(=O)NCCN)c(CCC(=O)OC)c4C)C1(C)C3=CC=C(C(=O)OC)C1C(=O)OC]

or a pharmaceutically acceptable salt thereof, a regioisomer, or a pharmaceutically acceptable salt of the regioisomer.

### Method of Uses and Compositions

The present disclosure also provides compositions (e.g., pharmaceutical compositions) comprising any photosensitizer-targeting agent conjugates described herein, such as those described in the fourth aspect or any embodiments described therein. In some embodiments, the pharmaceutical composition of the present disclosure comprises a photosensitizer-targeting agent conjugates described herein, such as those described in the fourth aspect or any embodiments described therein, and a pharmaceutically acceptable excipient (e.g., carriers, diluents, etc.).

The pharmaceutical compositions described herein can be administered in any number of ways for either local or systemic treatment. Administration can be topical (such as to mucous membranes including vaginal and rectal delivery) such as transdermal patches, ointments, lotions, creams, gels, drops, suppositories, sprays, liquids and powders; pulmonary (e.g., by inhalation or insufflation of powders or aerosols, including by nebulizer; intratracheal, intranasal, epidermal and transdermal); oral; or parenteral including intravenous, intraarterial, subcutaneous, intraperitoneal or intramuscular injection or infusion; or intracranial (e.g., intrathecal or intraventricular) administration. In some particular embodiments, the administration is intravenous. The pharmaceutical compositions described herein can also be used in vitro or in ex vivo.

Suitable pharmaceutically acceptable carriers, diluents, and excipients are well known in the art (see, e.g., Remington: The Science and Practice of Pharmacy, 21 st ed., 2005; and the books in the series Drugs and the Pharmaceutical Sciences: a Series of Textbooks and Monographs (Dekker, NY)) and can be determined by those of ordinary skill in the art as the clinical situation warrants. Examples of suitable carriers, diluents and/or excipients include: (1) Dulbecco's phosphate buffered saline, pH about 7.4, containing or not containing about 1 mg/mL to 25 mg/mL human serum albumin, (2) 0.9% saline (0.9% w/v NaCl), and (3) 5% (w/v) dextrose; and may also contain an antioxidant such as tryptamine and a stabilizing agent such as Tween 20.

The photosensitizer-targeting agent conjugates or compositions (e.g., pharmaceutical compositions) comprising the conjugates can be used for treating various diseases or conditions.

In some embodiments, the present disclosure provides a method of treating a subject having a disease or condition comprising: a) administering to the subject an effective amount of the conjugate described herein (e.g., those described in the fourth aspect or any embodiments described therein), or a pharmaceutically acceptable salt thereof, or a pharmaceutical composition comprising the conjugate or a pharmaceutically acceptable salt thereof; and b) after administering the conjugate or a pharmaceutically acceptable salt thereof or a pharmaceutical comprising the conjugate or a pharmaceutically acceptable salt thereof, illuminating a target region of the subject with a near infrared (NIR) light sufficient to activate the photosensitizer of the conjugate. In some embodiments, the targeting agent (e.g., an antibody or an antigen binding fragment thereof) in the conjugate binds to a target molecule of one or more cells located in the targeting region. In some embodiments, the targeting agent (e.g., an antibody or an antigen binding fragment thereof) in the conjugate binds to a target molecule of one or more cells located in the vicinity of the targeting region.

In some embodiments, the target region is a tumor cell, a mass of tumor cells, a solid tumor, in the vicinity of a solid tumor, a metastasis, a metastasized tumor cell, in the vicinity of a metastasis or a pre-cancerous lesion. In some embodiments, the disease or condition that can be treated by the present methods is a cancer. In some embodiments, the cancer is selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, esophogeal, gastrointestinal cancer, stomach (gastric) cancer, cancer of the small intestine, colon cancer, spindle cell neoplasm, hepatic carcinoma, liver cancer, cholangiocarcinoma, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

As used herein, the term “in the vicinity” refers to a distance where a near infrared (NIR) light can deliver a dose of illumination sufficient to activate the photosensitizer of the conjugate. The distance may depend on the light source and/or wavelength of the light. In some embodiments, the distance is 30 cm, 25 cm, 20 cm, 15 cm, 10 cm, 8 cm or 5 cm from the target cell or tissue.

In some embodiments, the target region is a skin lesion. In some embodiments, the disease or condition that can be treated by the present methods is skin lesions associated with psoriasis, atopic dermatitis, lupus, vitiligo, graft-versus-host disease, cutaneous T-cell lymphoma, contact dermatitis, cutaneous hypersensitivity response, lichen planus, lichen planopilaris, rejection of vascularized composite allografts, alopecia areata, scarring alopecia or sarcoid.

In some embodiments, the target region is kidney of the subject. In some embodiments, the disease or condition that can be treated by the present methods is T cell mediated kidney or renal pathology/disease associated with lupus nephritis, autoimmune nephritis or kidney graft rejection.

In some embodiments, the target region is gut or bowel of the subject. In some embodiments, the disease or condition that can be treated by the present methods is gastrointestinal inflammation as a result of an autoimmune and/or an inflammatory condition, e.g., inflammatory bowel disease (Crohn's disease, ulcerative colitis).

In some embodiments, the target region is inflamed joints of the subject. In some embodiments, the disease or condition that can be treated by the present methods is joint inflammation from rheumatoid arthritis or spondyloarthritides.

In some embodiments, the present disclosure provides a method for imaging a cell or tissue having a target molecule in a subject, the method comprising: a) administering to the subject an effective amount of the conjugate described herein (e.g., those described in the fourth aspect or any embodiments described therein), or a pharmaceutically acceptable salt thereof, or a pharmaceutical composition comprising the conjugate or a pharmaceutically acceptable salt thereof; and b) after administering the conjugate or a pharmaceutically acceptable salt thereof or a pharmaceutical comprising the conjugate or a pharmaceutically acceptable salt thereof, illuminating a target region of the subject with a near infrared (NIR) light sufficient to activate the photosensitizer of the conjugate, thereby providing an image of the presence of the target molecule associated with the cell or tissue. In some embodiments, the targeting agent (e.g., an antibody or an antigen binding fragment thereof) in the conjugate binds to the target molecule of the cell or tissue to be imaged. In some embodiments, the cell or tissue is located within the targeting region. In some embodiments, the cell or tissue is located in the vicinity the targeting region.

In some embodiments, the target region is a tumor cell, a mass of tumor cells, a solid tumor, in the vicinity of a solid tumor, a metastasis, a metastasized tumor cell, in the vicinity of a metastasis or a pre-cancerous lesion. In some embodiments, the target region is skin legion. In some embodiments, the target region is kidney of the subject. In some embodiments, the target region is gut or bowel of the subject. In some embodiments, the target region is inflamed joints of the subject.

Any suitable NIR light can be used in the methods described above. In some embodiments, NIR light is at a wavelength between 600 nm to 850 nm. In some embodiments, the NIR light is at a wavelength between 660 nm to 740 nm, e.g, 660 nm, 670 nm, 680 nm, 690 nm, 700 nm, 710 nm, 720 nm, 730 nm, or 740 nm. The NIR light can be adminstered using any methods known in the art, e.g, therapeutic laser.

In some embodiments, the target region can be illuminated at a dose sufficient to activate the photosensitizer of the conjugate. In some embodiments, the dose of illumination is from about 1 J/cm² to about 400 J/cm². In some embodiments, the dose of illumination is from about 2 J/cm² to about 400 J/cm². In some embodiments, the dose of illumination is from about 1 J/cm² to about 300 J/cm². In some embodiments, the dose of illumination is from about 10 J/cm² to about 100 J/cm². In some embodiments, the dose of illumination is from about 10 J/cm² to about 50 J/cm².

In some embodiments, for the methods described above, the illumination is performed at least 5 minutes after administration of the conjugate. In some embodiments, the illumination is performed between 1 hour and 48 hours after administration of the conjugate. In some embodiments, the illumination is performed between 4 hours and 30 hours after administration of the conjugate. In some embodiments, the illumination is performed between 1 hours and 24 hour after administration of the conjugate. In some embodiments, the illumination is performed between 8 hours and 24 hour after administration of the conjugate. In some embodiments, the illumination is performed 1 hour, 2 hours, 3 hours, 4 hours, 5 hours, 6 hours, 7 hours, 8 hours, 12 hours, 15 hours, 18 hours or 24 hours after administration of the conjugate.

### Numbered Embodiments

Embodiment 1. A compound represented by the following formula:

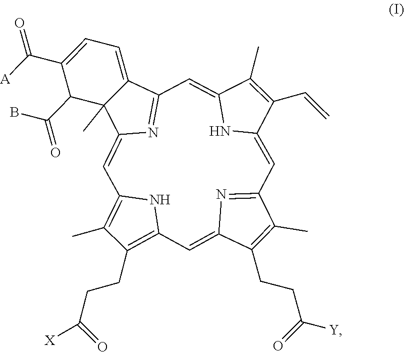

or a pharmaceutically acceptable salt thereof, wherein:
A, B, X and Y are each independently —OH, —OC₁₋₄alkyl, —N(R¹⁰⁰)(R¹⁰¹), or —Z-L^{P}-ker, provided at least one of A, B, X and Y is —N(R¹⁰⁰)(R¹⁰¹), —Z-L^{P}-NH₂, or —Z-L^{P}-ker, and provided at most one of A, B, X and Y is —Z-L^{P}-NH₂ or —Z-L^{P}-ker;
R¹⁰⁰ is H or C₁₋₃alkyl;
R¹⁰¹ is H, C₁₋₃alkyl or —(CH₂—CH₂—O)ₙᵣR¹⁰²;
R¹⁰² is H or Me;
nr is an integer from 1 to 16;
Z is —O— or —NR¹—;
R¹ is H or C₁₋₃alkyl;
L^{P} is a spacer;
ker is a reactive group.

Embodiment 2. The compound of embodiment 1, or a pharmaceutically acceptable salt thereof, wherein:
A, B, X and Y are each independently —OH, —OC₁₋₄alkyl, —N(R¹⁰⁰)(R¹⁰¹), or —Z-L^{P}-ker, provided at least one of A, B, X and Y is —N(R¹⁰⁰)(R¹⁰¹) or —Z-L^{P}-ker, and provided at most one of A, B, X and Y is —Z-L^{P}-ker;
R¹⁰⁰ is H or C₁₋₃alkyl;
R¹⁰¹ is H, C₁₋₃alkyl or —(CH₂—CH₂—O)ₙᵣR¹⁰²;
R¹⁰² is H or Me;
nr is an integer from 1 to 16;
Z is —O— or —NR¹—;
R¹ is H or C₁₋₃alkyl;
L^{P} is a spacer;
ker is a reactive group.

Embodiment 3. The compound of embodiment 1, or a pharmaceutically acceptable salt thereof, wherein:
a) A and B are —OCH₃; and one of X and Y is —OCH₃ and the other one of X and Y is —Z-L^{P}-ker;
b) A and B are —OCH₃; and one of X and Y is —OH and the other one of X and Y is —Z-L^{P}-ker;
c) one of A and B is —OCH₃, and the other is —OH; and one of X and Y is —OH and the other one of X and Y is —Z-L^{P}-ker;
d) A and B are —OH; and one of X and Y is —OH and the other one of X and Y is —Z-L^{P}-ker;
e) one of A and B is —OH and the other one of A and B is —OH or —OCH₃; and X and Y are both —NH(CH₂—CH₂—O)ₙᵣR¹⁰², wherein R¹⁰² is H or Me; and nr is an integer from 2 to 8; or
f) one of A and B is —Z-L^{P}-ker and the other one of A and B is —OH or —OCH₃; and X and Y are both —NH(CH₂—CH₂—O)ₙᵣR¹⁰², wherein R¹⁰² is H or Me; and nr is an integer from 2 to 8.

Embodiment 4. The compound of embodiment 1, or a pharmaceutically acceptable salt thereof, wherein the compound is represented by the following formula:

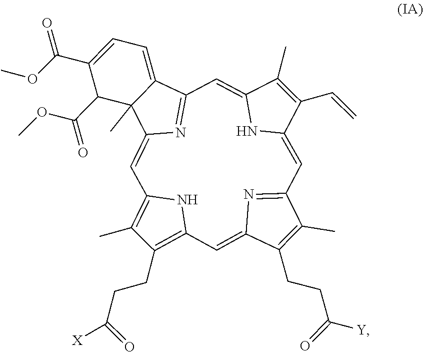

or a pharmaceutically acceptable salt thereof, wherein one of X and Y is —Z-L^{P}-ker and other is —OH.

Embodiment 5. The compound of embodiment 1, or a pharmaceutically acceptable salt thereof, wherein the compound is represented by the following formula:

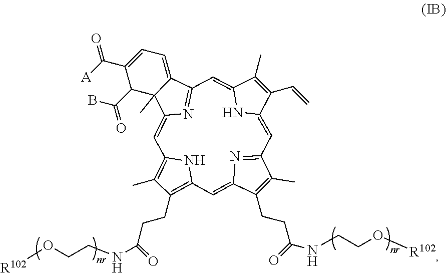

or a pharmaceutically acceptable salt thereof, wherein one of A and B is —OH or —Z-L^{P}-ker and the other is —OH or —OCH₃; R¹⁰² is H or Me; and nr is an integer from 2 to 8.

Embodiment 6. The compound of any one of embodiments 1-5, or a pharmaceutically acceptable salt thereof, wherein ker is a reactive group selected from: a maleimide group, a thiol group, a cyclooctyne group, an azido group, a hydrazide group, a tetrazine group, cyclooctene group, a ketone group, and an aldehyde group.

Embodiment 7. The compound of any one of embodiments 1-5, or a pharmaceutically acceptable salt thereof, wherein ker is

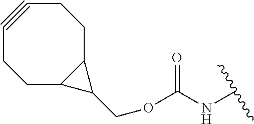 [SMILES: *NC(=O)OCC1C2CCC#CCCC21]

### or —N₃.

Embodiment 8. The compound of any one of embodiments 1-7, or a pharmaceutically acceptable salt thereof, wherein Z is —NH—.

Embodiment 9. The compound of any one of embodiments 1-7, or a pharmaceutically acceptable salt thereof, wherein Z is —O—.

Embodiment 10. The compound of any one of embodiments 1-9, or a pharmaceutically acceptable salt thereof, wherein L^{P} is a non-cleavable spacer.

Embodiment 11. The compound of embodiment 10, or a pharmaceutically acceptable salt thereof, wherein L^{P} comprises a hydrophilic moiety.

Embodiment 12. The compound of embodiment 10, or a pharmaceutically acceptable salt thereof, wherein L^{P} comprises a PEG moiety.

Embodiment 13. The compound of embodiment 10, or a pharmaceutically acceptable salt thereof, wherein L^{P} is *—CH₂CH₂—(OCH₂CH₂)ₘ—, wherein * is the site connected to ker, and m is an integer from 0 to 30.

Embodiment 14. The compound of embodiment 13, or a pharmaceutically acceptable salt thereof, m is an integer from 2 to 16, from 2 to 10, from 2 to 8, or from 3 to 5; or m is 2, 3, 4, 5, 6, 7 or 8.

Embodiment 15. The compound of embodiment 10, or a pharmaceutically acceptable salt thereof, wherein L^{P} is *—CH₂CH₂—(OCH₂CH₂)₄—.

Embodiment 16. The compound of any one of embodiments 1-9, or a pharmaceutically acceptable salt thereof, wherein L^{P} is a cleavable spacer.

Embodiment 17. The compound of embodiment 16, or a pharmaceutically acceptable salt thereof, wherein the cleavable spacer comprises an enzyme labile group, a pH labile group, a disulfide group, a ROS cleavable group, a photocleavable group.

Embodiment 18. The compound of embodiment 17, or a pharmaceutically acceptable salt thereof, wherein the enzyme labile group is a group cleavable by an esterase, a peptidase, an aminopeptidase, a β-galactosidase, a β-glucuronidase, a carboxylesterase, a caspase, a diaphorase, a histone deacetylase, a legumain, or matrix metalloproteinase.

Embodiment 19. The compound of embodiment 18, or a pharmaceutically acceptable salt thereof, wherein the enzyme labile group is a group cleavable by cathepsin B.

Embodiment 20. The compound of embodiment 16, or a pharmaceutically acceptable salt thereof, wherein the spacer represented by L^{P} comprises a peptide and a self-immolative group.

Embodiment 21. The compound of embodiment 20, or a pharmaceutically acceptable salt thereof, wherein the spacer L^{P} is presented by the following formula:

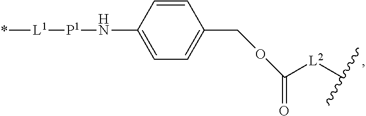

wherein:
*- represents a bond to the reactive group represented by ker;

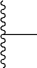

represents a bond to the group Z;
L¹ and L² are each independently a connecting spacer;
P¹ is a peptide comprising 2 to 5 amino acid residues.

Embodiment 22. The compound of embodiment 21, or a pharmaceutically acceptable salt thereof, wherein P¹ is a peptide selected from Phe-Arg-Arg-Gly, Glu-Val-Cit, Val-Cit, Cit-Val, Gly-Gly-Phe, Val-Ala, and Ala-Val.

Embodiment 23. The compound of embodiment 22, or a pharmaceutically acceptable salt thereof, wherein P¹ is:

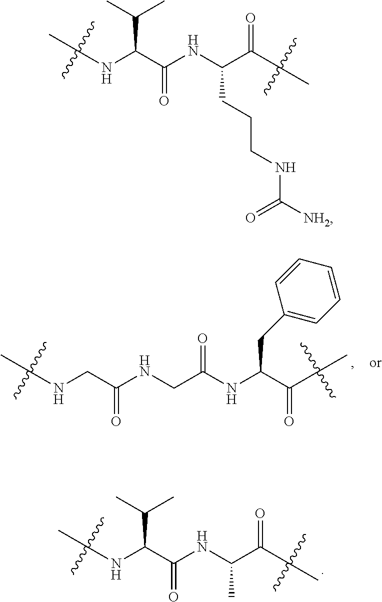

Embodiment 24. The compound of any one of embodiments 21-23, or a pharmaceutically acceptable salt thereof, wherein L¹ comprises a hydrophilic moiety.

Embodiment 25. The compound of embodiment 24, or a pharmaceutically acceptable salt thereof, wherein L¹ comprises a PEG moiety.

Embodiment 26. The compound of any one of embodiments 21-23, or a pharmaceutically acceptable salt thereof, wherein L¹ is *—CH₂—CH₂—(OCH₂CH₂)ₙ—C(═O)—**; n is an integer from 0 to 30; *- represents a bond to the reactive group represented by ker; and **- represents a bond to P¹.

Embodiment 27. The compound of embodiment 26, or a pharmaceutically acceptable salt thereof, wherein n is an integer from 2 to 16, from 2 to 10, from 2 to 8, or from 3 to 5; or n is 2, 3, 4, 5, 6, 7 or 8.

Embodiment 28. The compound of embodiment 26, or a pharmaceutically acceptable salt thereof, wherein L¹ is *—CH₂—CH₂—(OCH₂CH₂)₃—C(═O)—**.

Embodiment 29. The compound of any one of embodiments 21-28, or a pharmaceutically acceptable salt thereof, wherein L² is —NH—C₁₋₆alkyl-**, wherein ** is the site connected to Z.

Embodiment 30. The compound of embodiment 29, or a pharmaceutically acceptable salt thereof, wherein L² is —NH—CH₂CH₂—**; wherein ** is the site connected to Z.

Embodiment 31. The compound of embodiment 1, or a pharmaceutically acceptable salt thereof, wherein the compound is:

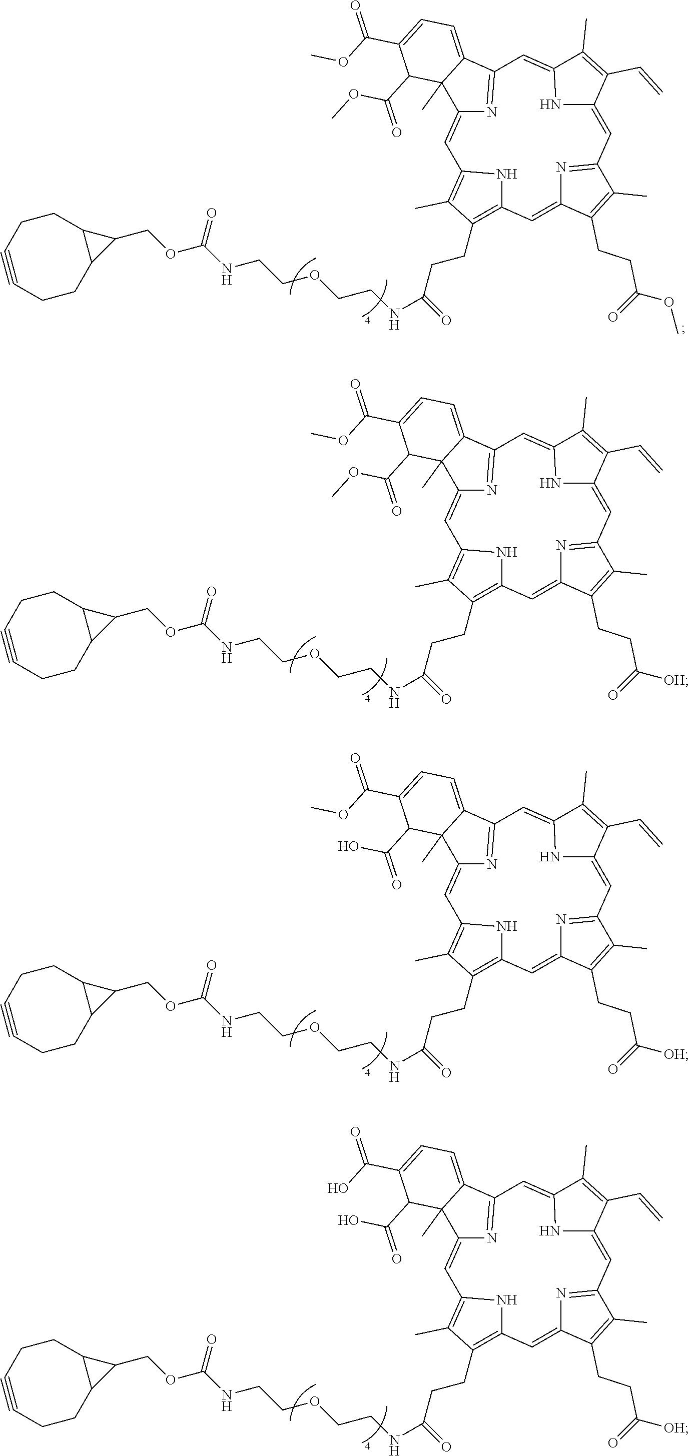

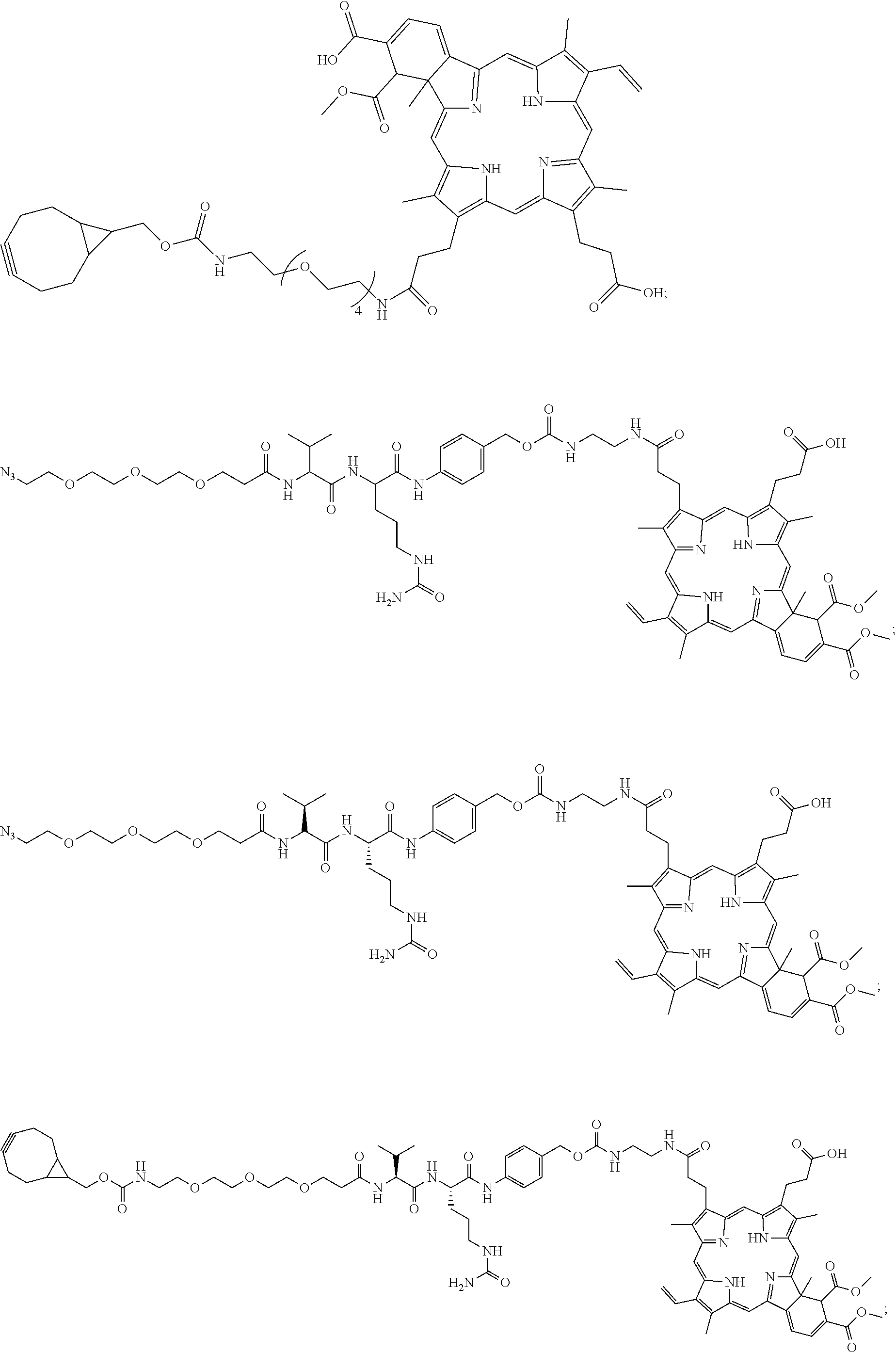

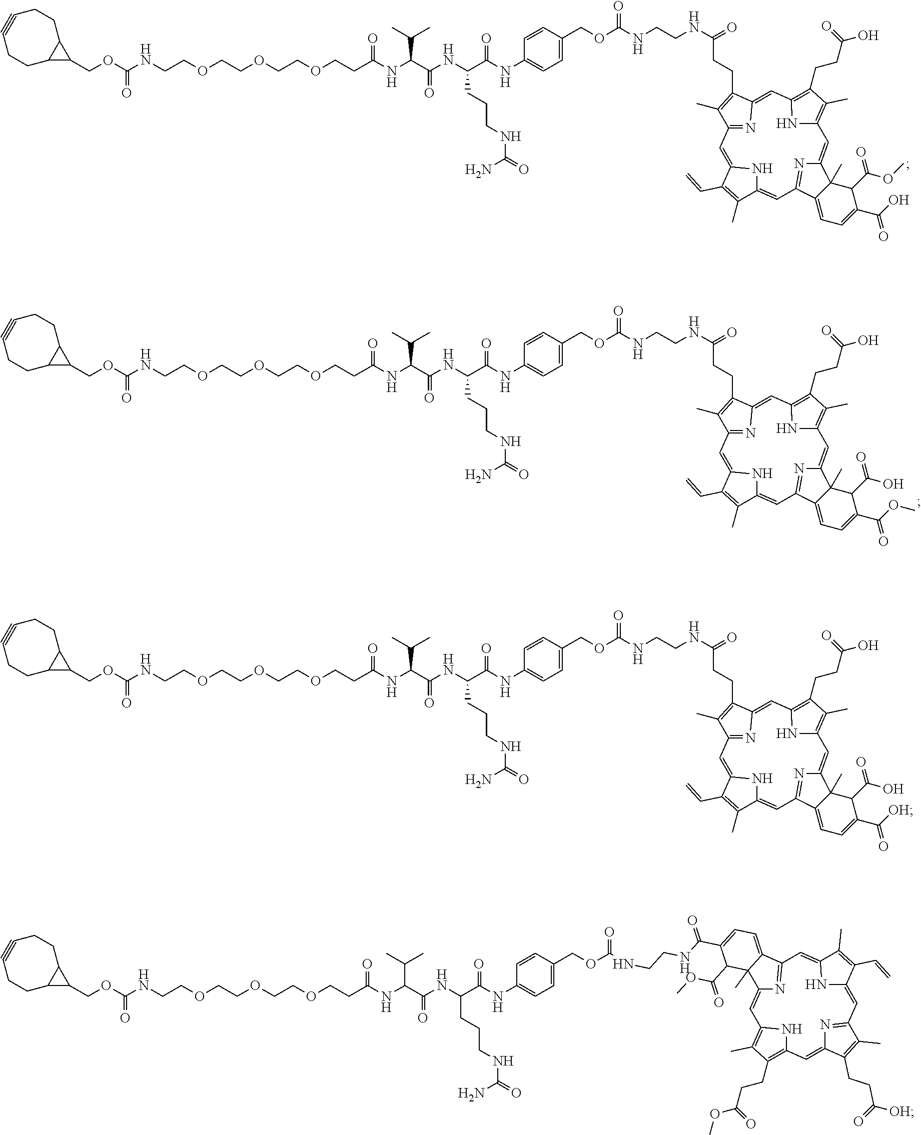

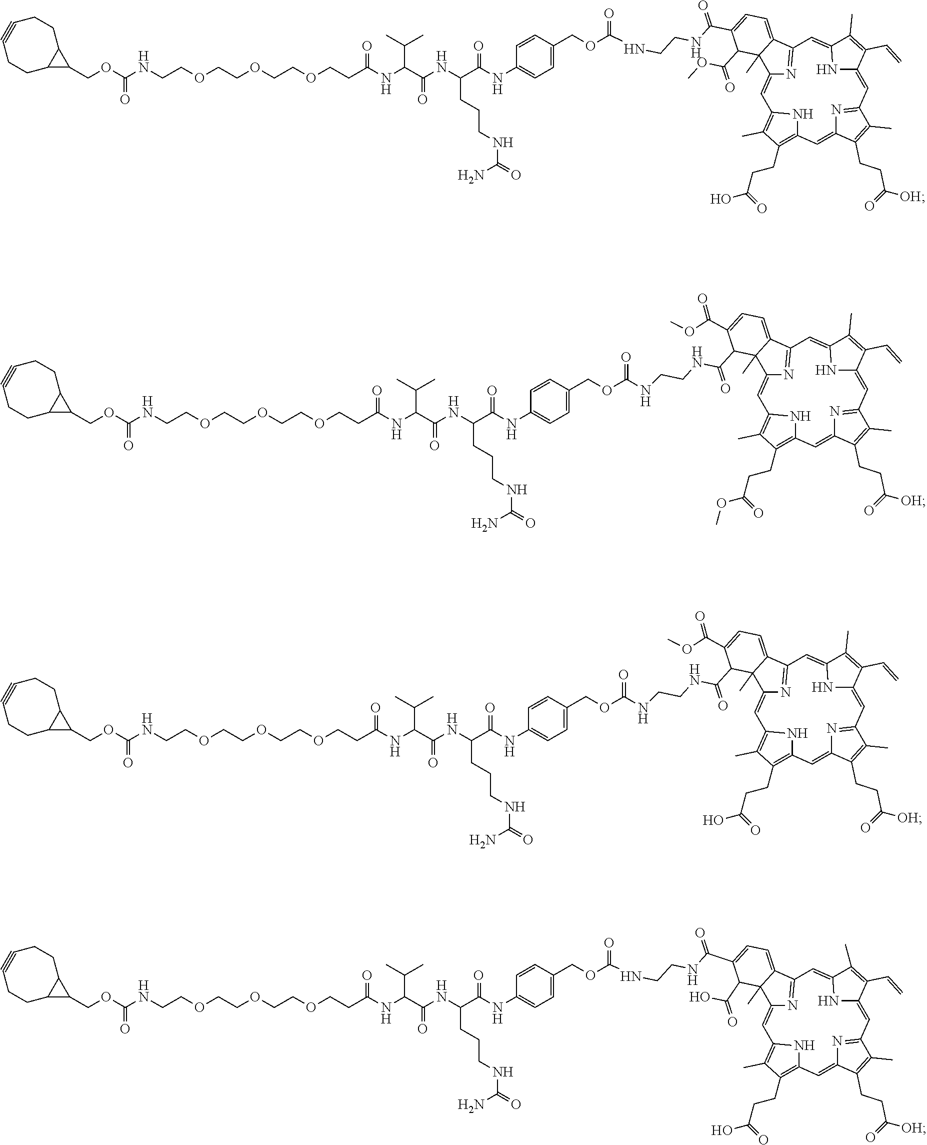

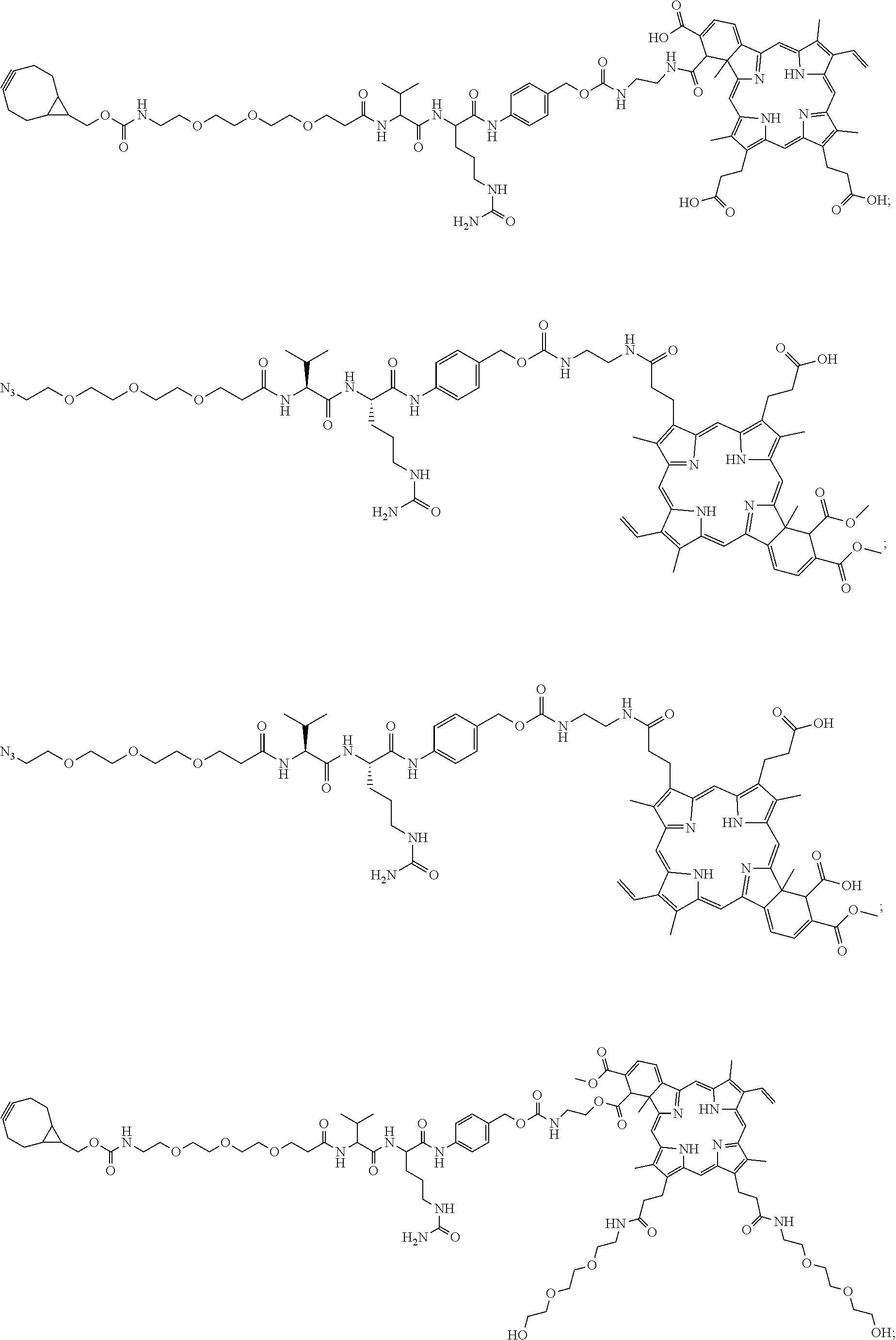

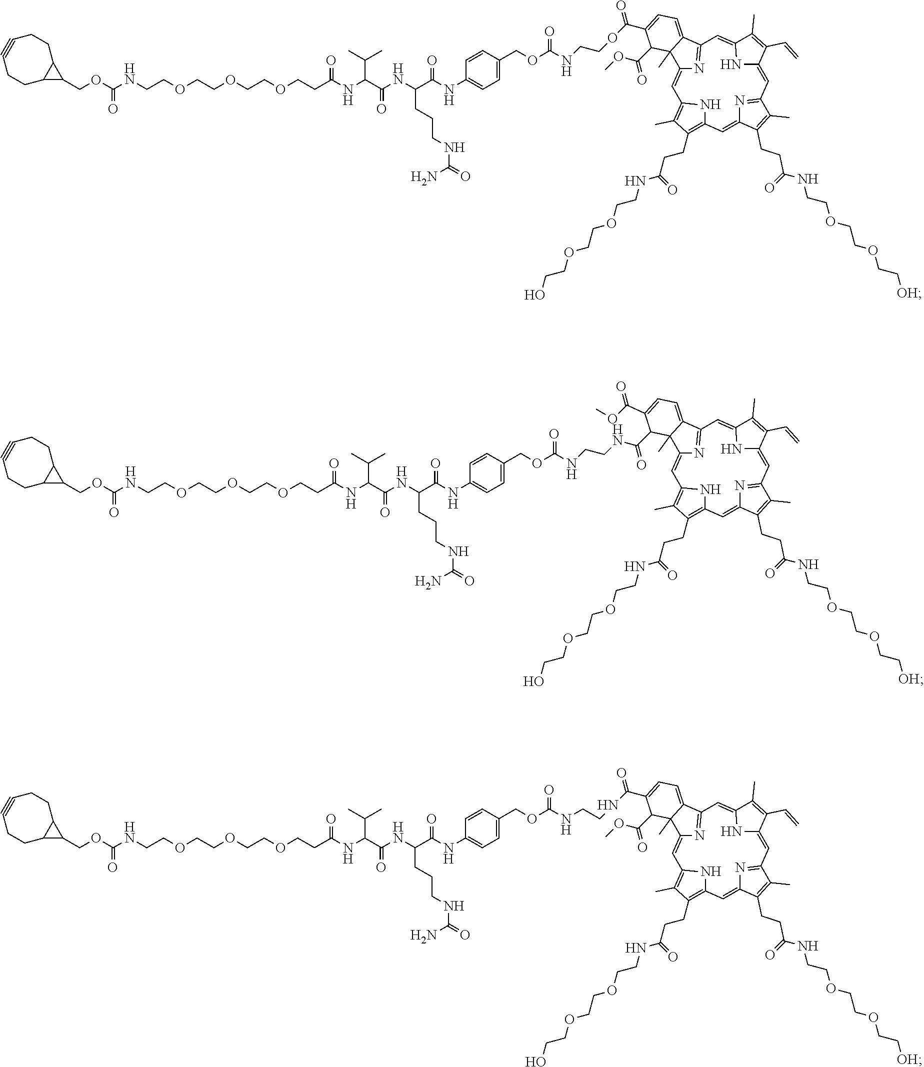

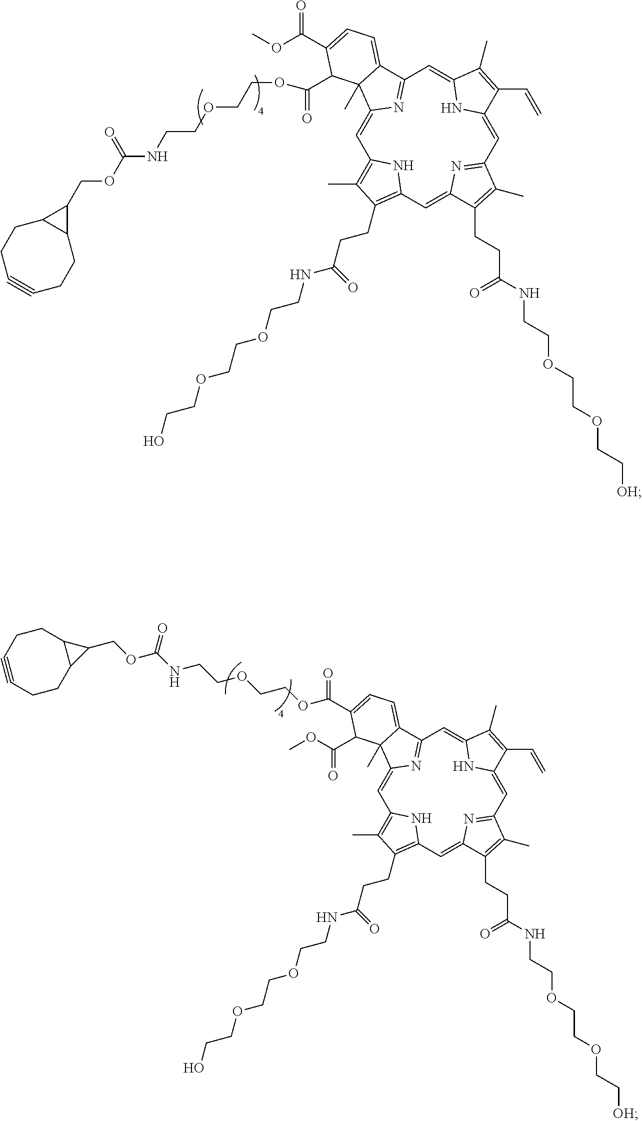

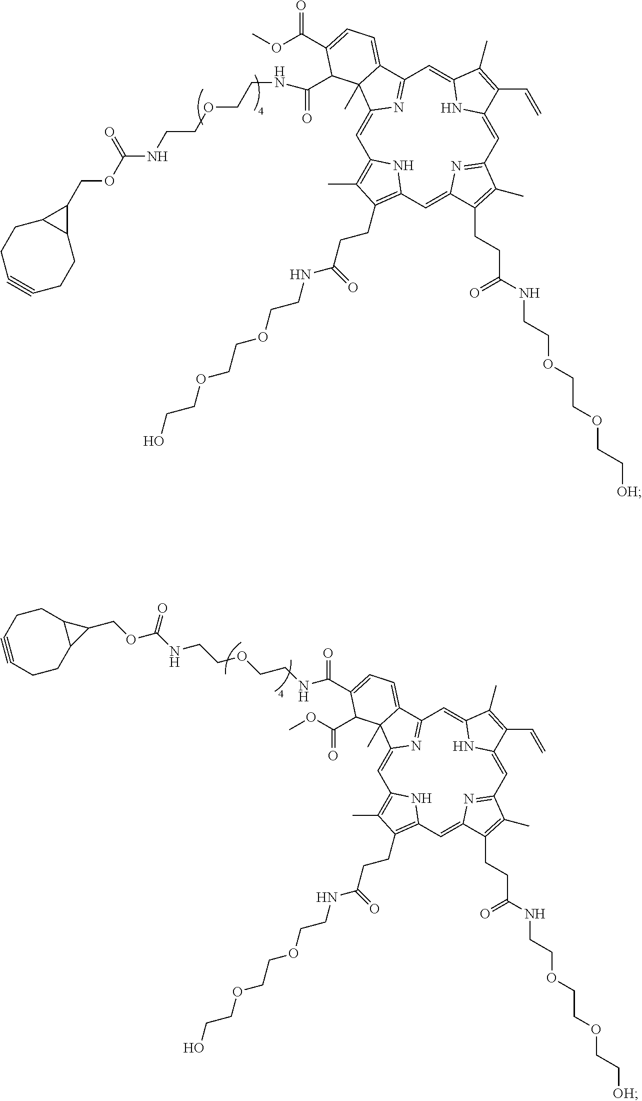

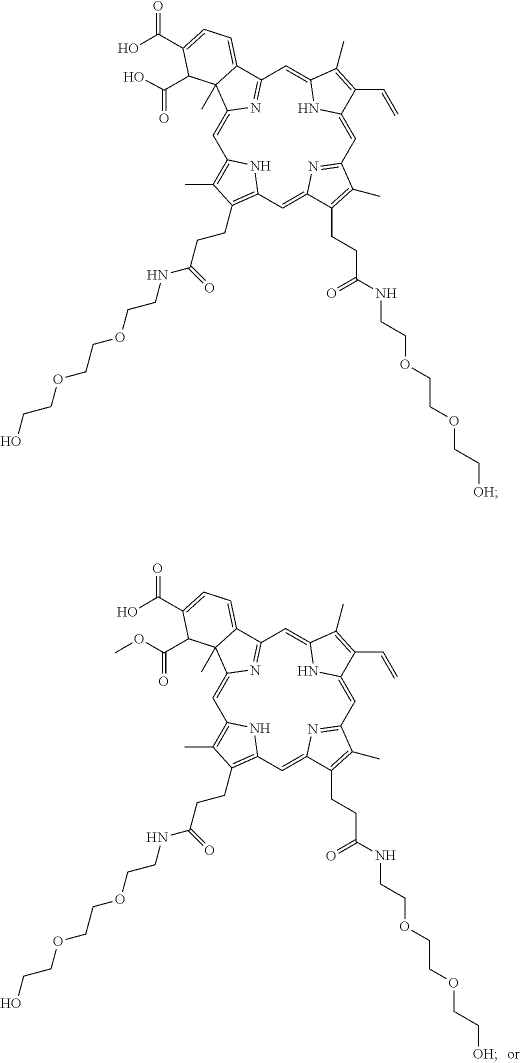 [SMILES: C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)O)C1C(=O)O ; C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)O)C1C(=O)OC]

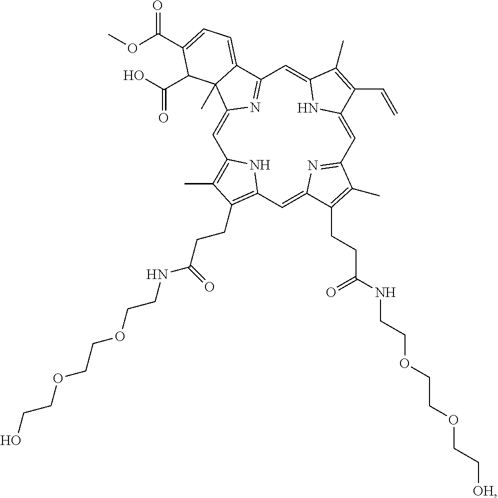 [SMILES: C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)OC)C1C(=O)O]

or a pharmaceutically acceptable salt thereof, a regioisomer, or a pharmaceutically acceptable salt of the regioisomer.

Embodiment 32. The compound of embodiment 1, or a pharmaceutically acceptable salt thereof, wherein the compound is:

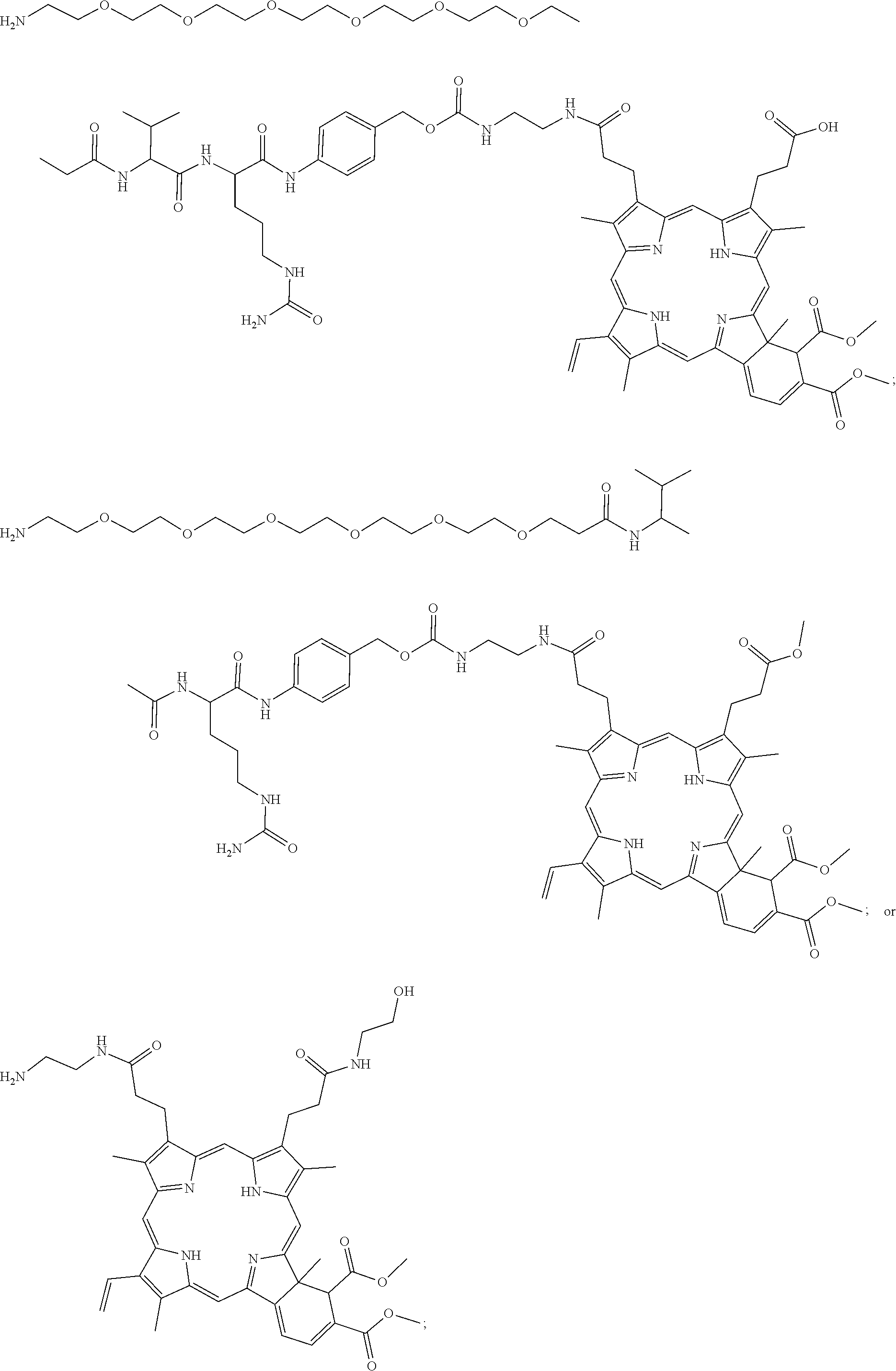

or a pharmaceutically acceptable salt thereof, a regioisomer, or a pharmaceutically acceptable salt of the regioisomer.

Embodiment 33. A linker-photosensitizer compound of formula (II):

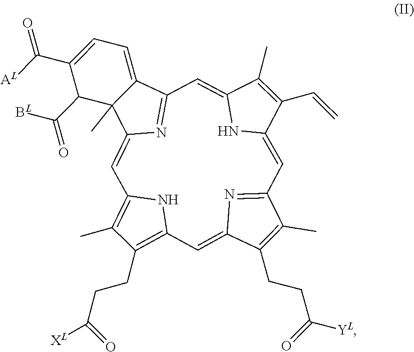

or a pharmaceutically acceptable salt thereof, wherein:
A^{L}, B^{L}, X^{L} and Y^{L} are each independently —OH, —OC₁₋₄alkyl, —N(R¹⁰⁰)(R¹⁰¹) or —Z-L^{P}-linker-L^{A}-R^{A}, provided one of A^{L}, B^{L}, X^{L} and Y^{L} is —Z-L^{P}-linker-L^{A}-R^{A};
R¹⁰⁰ is H or C₁₋₃alkyl;
R¹⁰¹ is H, C₁₋₃alkyl or —(CH₂—CH₂—O)ₙᵣR¹⁰²;
R¹⁰² is H or Me;
nr is an integer from 1 to 16;
Z is —O— or —NR¹—;
R¹ is H or C₁₋₃alkyl;
L^{P} and L^{A} are each independently a spacer;
Linker is connection moiety formed by a first reactive group attached to L^{P} and a second reactive group attached to L^{A}; and
R^{A} is a reactive group that can form a covalent bond with a targeting agent.

Embodiment 34. The compound of embodiment 33, or a pharmaceutically acceptable salt thereof, wherein R^{A} is a reactive ester, —NH₂, or a maleimide group.

Embodiment 35. The compound of embodiment 33, wherein R^{A} is

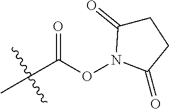 [SMILES: *C(=O)ON1C(=O)CCC1=O]

### or —NH₂.

Embodiment 36. The compound of any one of embodiments 33-35, or a pharmaceutically acceptable salt thereof, wherein L^{A} is a spacer comprising a hydrophilic moiety.

Embodiment 37. The compound of embodiment 36, or a pharmaceutically acceptable salt thereof, wherein L^{A} is a spacer comprising a PEG moiety.

Embodiment 38. The compound of embodiment 36, or a pharmaceutically acceptable salt thereof, wherein L^{A} is *—CH₂CH₂(OCH₂CH₂)ₚ—, wherein * is the site connected to R^{A}; and p is an integer from 0 to 30.

Embodiment 39. The compound of embodiment 37, or a pharmaceutically acceptable salt thereof, wherein p is an integer from 2 to 16, from 2 to 10, from 2 to 8, or from 3 to 5; or p is 2, 3, 4, 5, 6, 7 or 8.

Embodiment 40. The compound of any one of embodiments 33-39, wherein the first reactive group and the second reactive group are each selected from a maleimide group, a thiol group, a cyclooctyne group, and an azido group.

Embodiment 41. The compound of embodiment 40, wherein Linker is represented by the following formula:

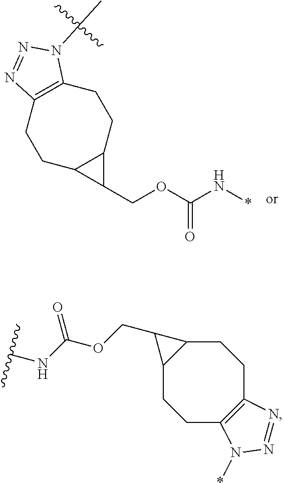 [SMILES: *NC(=O)OCC1C2CCc3nnn(*)c3CCC21 ; *NC(=O)OCC1C2CCc3nnn(*)c3CCC21]

wherein *- represents a bond to L^{P} and

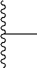

represents a bond to L^{A}.

Embodiment 42. The compound of any one of embodiments 33-41, or a pharmaceutically acceptable salt thereof, wherein:
a) A^{L} and B^{L} are —OCH₃; and one of X^{L} and Y^{L} is —OCH₃ and the other one of X^{L} and Y^{L} is —Z-L^{P}-linker-L^{A}-R^{A}.
b) A^{L} and B^{L} are —OCH₃; and one of X^{L} and Y^{L} is —OH and the other one of X^{L} and Y^{L} is —Z-L^{P}-linker-L^{A}-R^{A}.
c) one of A^{L} and B^{L} is —OCH₃, and the other one of A^{L} and B^{L} is —OH; and one of X^{L} and Y^{L} is —OH and the other one of X^{L} and Y^{L} is —Z-L^{P}-linker-L^{A}-R^{A};
d) A^{L} and B^{L} are —OH; and one of X^{L} and Y^{L} is —OH and the other one of X^{L} and Y^{L} is —Z-L^{P}-linker-L^{A}-R^{A}; or
e) one of A^{L} and B^{L} is —Z-L^{P}-linker-L^{A}-R^{A} and the other one of A^{L} and B^{L} is —OH or —OCH₃; and X^{L} and Y^{L} are both —NH(CH₂—CH₂—O)ₙᵣR¹⁰², wherein R¹⁰² is H or Me; and nr is an integer from 2 to 8.

Embodiment 43. The compound of embodiment 42, or a pharmaceutically acceptable salt thereof, wherein the compound is represented by the following formula:

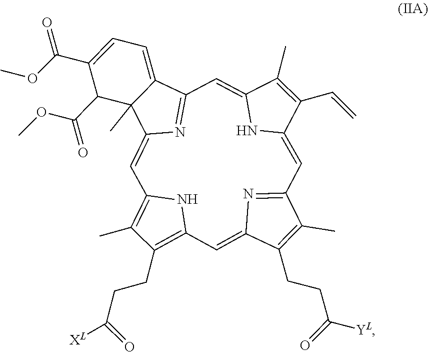

or a pharmaceutically acceptable salt thereof, wherein one of X^{L} and Y^{L} is —Z-L^{P}-linker-L^{A}-R^{A} and the other is —OH.

Embodiment 44. The compound of embodiment 42, or a pharmaceutically acceptable salt thereof, wherein the compound is represented by the following formula:

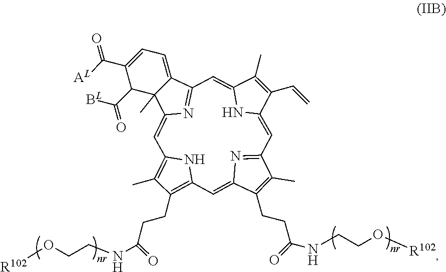

or a pharmaceutically acceptable salt thereof, wherein one of A^{L} and B^{L} is —Z-L^{P}-linker-L^{A}-R^{A} and the other is —OH or —OCH₃; R¹⁰² is H or Me; and nr is an integer from 2 to 8.

Embodiment 45. The compound of any one of embodiments 33-44, or a pharmaceutically acceptable salt thereof, wherein Z is —NH—.

Embodiment 46. The compound of any one of embodiments 33-44, or a pharmaceutically acceptable salt thereof, wherein Z is —O—.

Embodiment 47. The compound of any one of embodiments 33-46, or a pharmaceutically acceptable salt thereof, wherein L^{P} is a non-cleavable spacer.

Embodiment 48. The compound of embodiment 47, or a pharmaceutically acceptable salt thereof, wherein L^{P} comprises a hydrophilic moiety.

Embodiment 49. The compound of embodiment 48, or a pharmaceutically acceptable salt thereof, wherein L^{P} comprises a PEG moiety.

Embodiment 50. The compound of embodiment 49, or a pharmaceutically acceptable salt thereof, wherein L^{P} is *—CH₂CH₂—(OCH₂CH₂)ₘ—, wherein * is the site connected to the Linker, and m is an integer from 0 to 30.

Embodiment 51. The compound of embodiment 50, or a pharmaceutically acceptable salt thereof, m is an integer from 2 to 16, from 2 to 10, from 2 to 8, or from 3 to 5; or m is 2, 3, 4, 5, 6, 7 or 8.

Embodiment 52. The compound of embodiment 50, or a pharmaceutically acceptable salt thereof, wherein L^{P} is *—CH₂CH₂—(OCH₂CH₂)₄—, wherein * is the site connected to the Linker.

Embodiment 53. The compound of any one of embodiments 33-46, or a pharmaceutically acceptable salt thereof, wherein L^{P} is a cleavable spacer.

Embodiment 54. The compound of embodiment 53, or a pharmaceutically acceptable salt thereof, wherein the cleavable spacer comprises an enzyme labile group, a pH labile group, a disulfide group, a ROS cleavable group, a photocleavable group.

Embodiment 55. The compound of embodiment 54, or a pharmaceutically acceptable salt thereof, wherein the enzyme labile group is a group cleavable by an esterase, a peptidase, an aminopeptidase, a β-galactosidase, a β-glucuronidase, a carboxylesterase, a caspase, a diaphorase, a histone deacetylase, a legumain, or matrix metalloproteinase.

Embodiment 56. The compound of embodiment 55, or a pharmaceutically acceptable salt thereof, wherein the enzyme labile group is a group cleavable by cathepsin B.

Embodiment 57. The compound of embodiment 53, or a pharmaceutically acceptable salt thereof, wherein the spacer represented by L^{P} comprises a peptide and a self-immolative group.

Embodiment 58. The compound of embodiment 57, or a pharmaceutically acceptable salt thereof, wherein the spacer L^{P} is presented by the following formula:

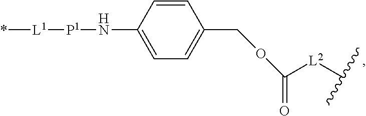

wherein:
*- represents a bond to the Linker group;

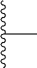

represents a bond to the group Z;
L¹ and L² are each independently a connecting spacer;
P¹ is a peptide comprising 2 to 5 amino acid residues.

Embodiment 59. The compound of embodiment 58, or a pharmaceutically acceptable salt thereof, wherein P¹ is a peptide selected from Phe-Arg-Arg-Gly, Glu-Val-Cit, Val-Cit, Cit-Val, Gly-Gly-Phe, Val-Ala, and Ala-Val.

Embodiment 60. The compound of embodiment 59, or a pharmaceutically acceptable salt thereof, wherein P¹ is:

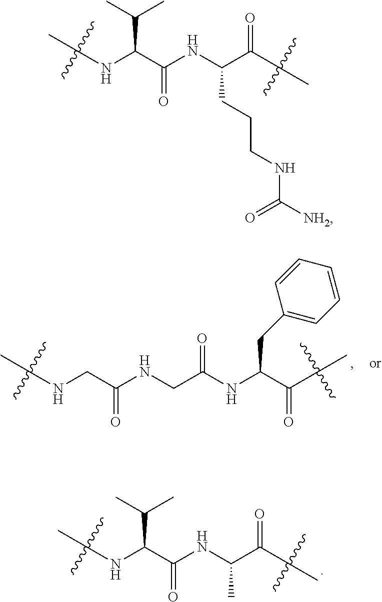

Embodiment 61. The compound of any one of embodiments 58-60, or a pharmaceutically acceptable salt thereof, wherein L¹ comprises a hydrophilic moiety.

Embodiment 62. The compound of embodiment 61, or a pharmaceutically acceptable salt thereof, wherein L¹ comprises a PEG moiety.

Embodiment 63. The compound of embodiment 62, or a pharmaceutically acceptable salt thereof, wherein L¹ is *—CH₂—CH₂—(OCH₂CH₂)ₙ—C(═O)—**; n is an integer from 0 to 30; *-represents a bond to the Linker group; and **- represents a bond to P¹.

Embodiment 64. The compound of embodiment 63, or a pharmaceutically acceptable salt thereof, wherein n is an integer from 2 to 16, from 2 to 10, from 2 to 8, or from 3 to 5; or n is 2, 3, 4, 5, 6, 7 or 8.

Embodiment 65. The compound of embodiment 63, or a pharmaceutically acceptable salt thereof, wherein L¹ is *—CH₂—CH₂—(OCH₂CH₂)₃—C(═O)—**, wherein *- represents a bond to the Linker group; and **- represents a bond to P¹.

Embodiment 66. The compound of any one of embodiments 58-68, or a pharmaceutically acceptable salt thereof, wherein L² is —NH—C₁₋₆alkyl-**, wherein ** is the site connected to Z.

Embodiment 67. The compound of embodiment 66, or a pharmaceutically acceptable salt thereof, wherein L² is —NH—CH₂CH₂—**, wherein ** is the site connected to Z.

Embodiment 68. The compound of embodiment 33, wherein the compound is represented by the following formula:

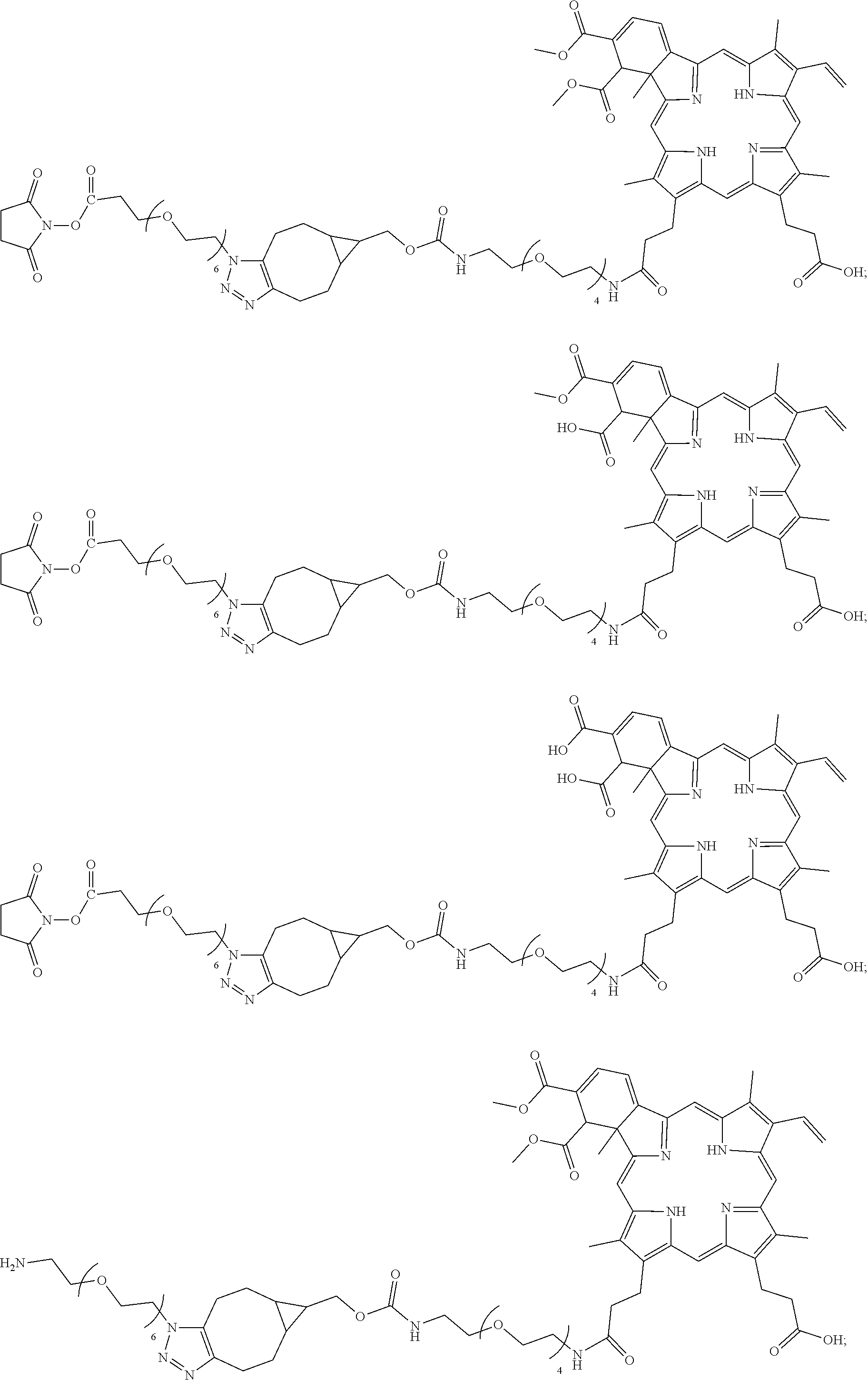

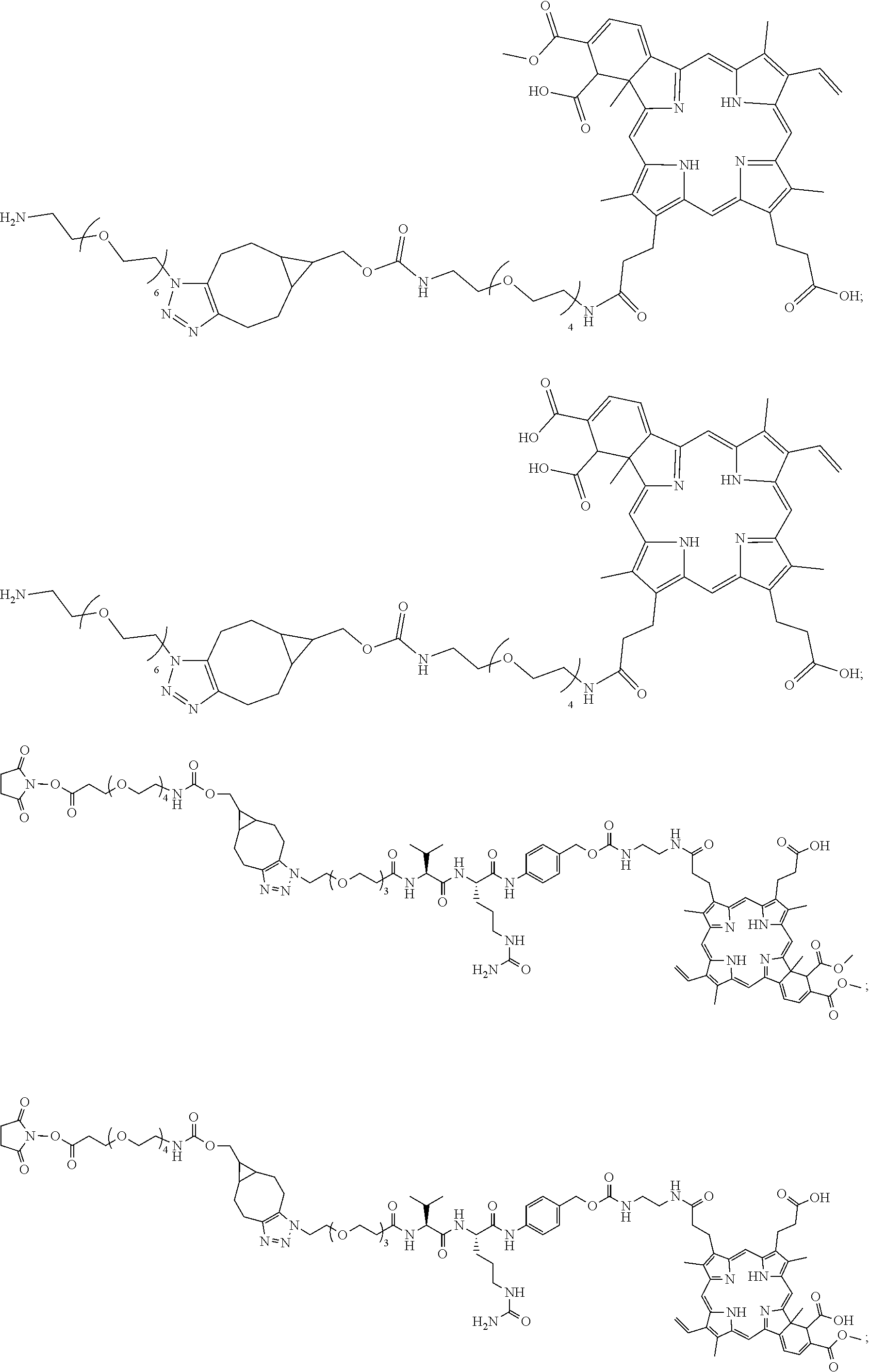

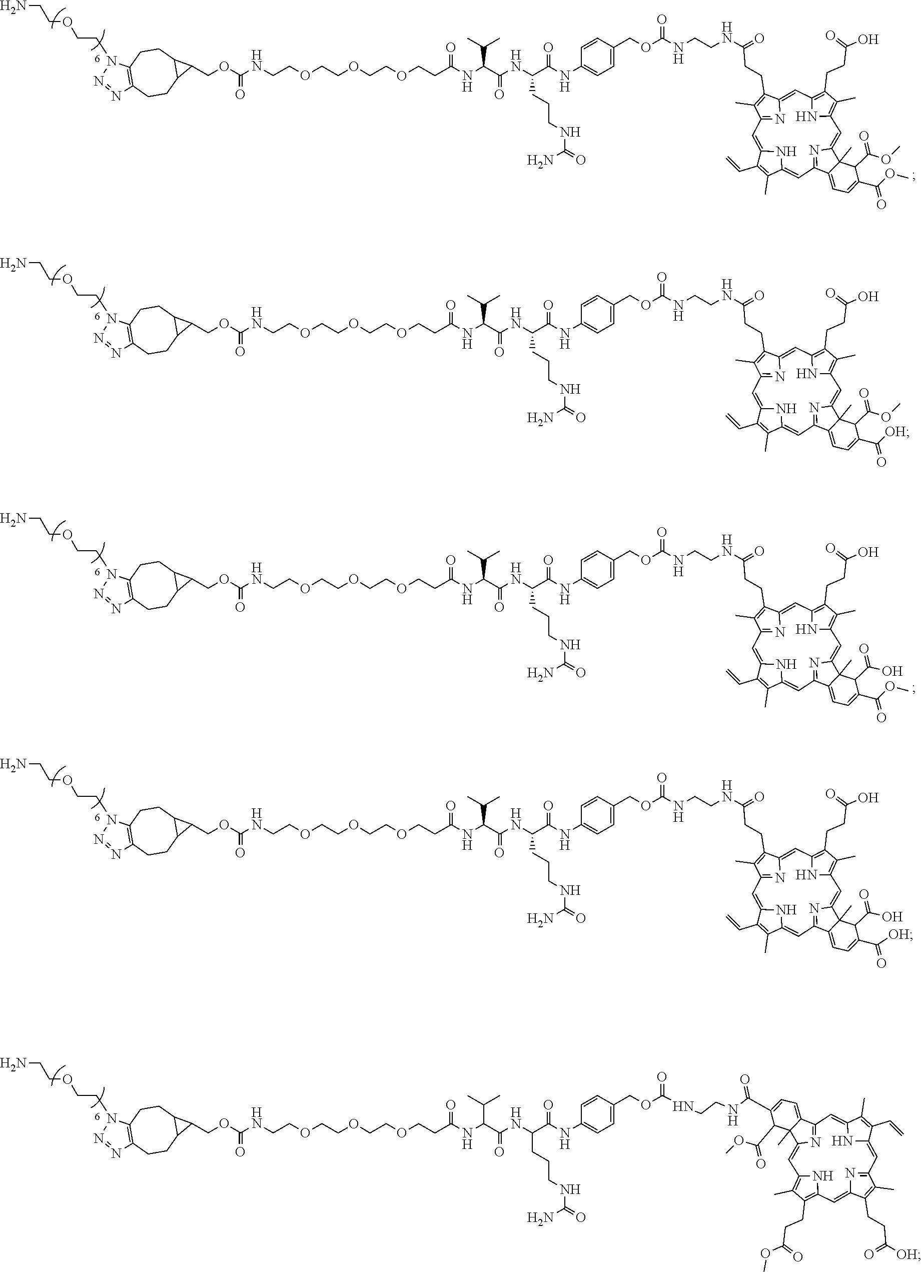

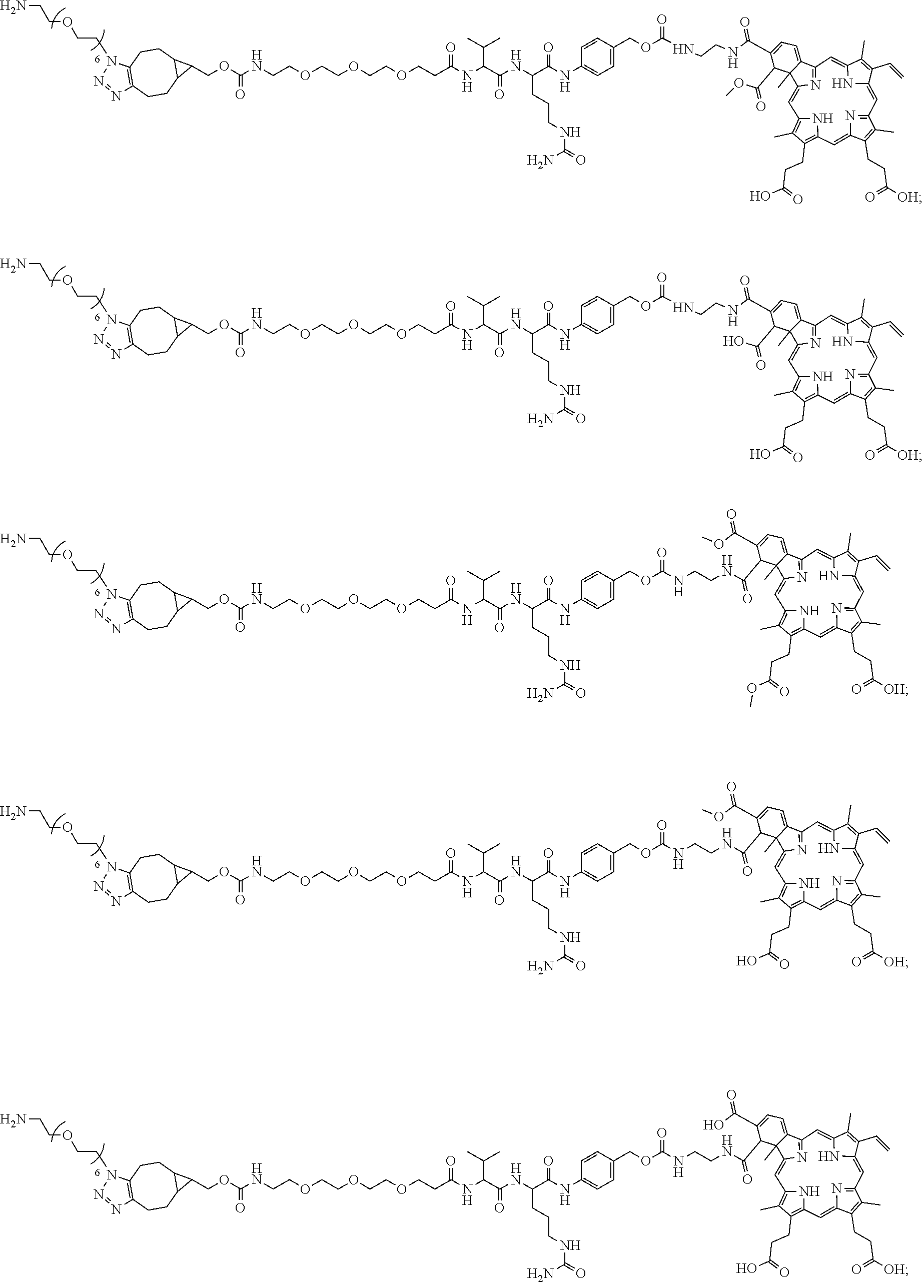

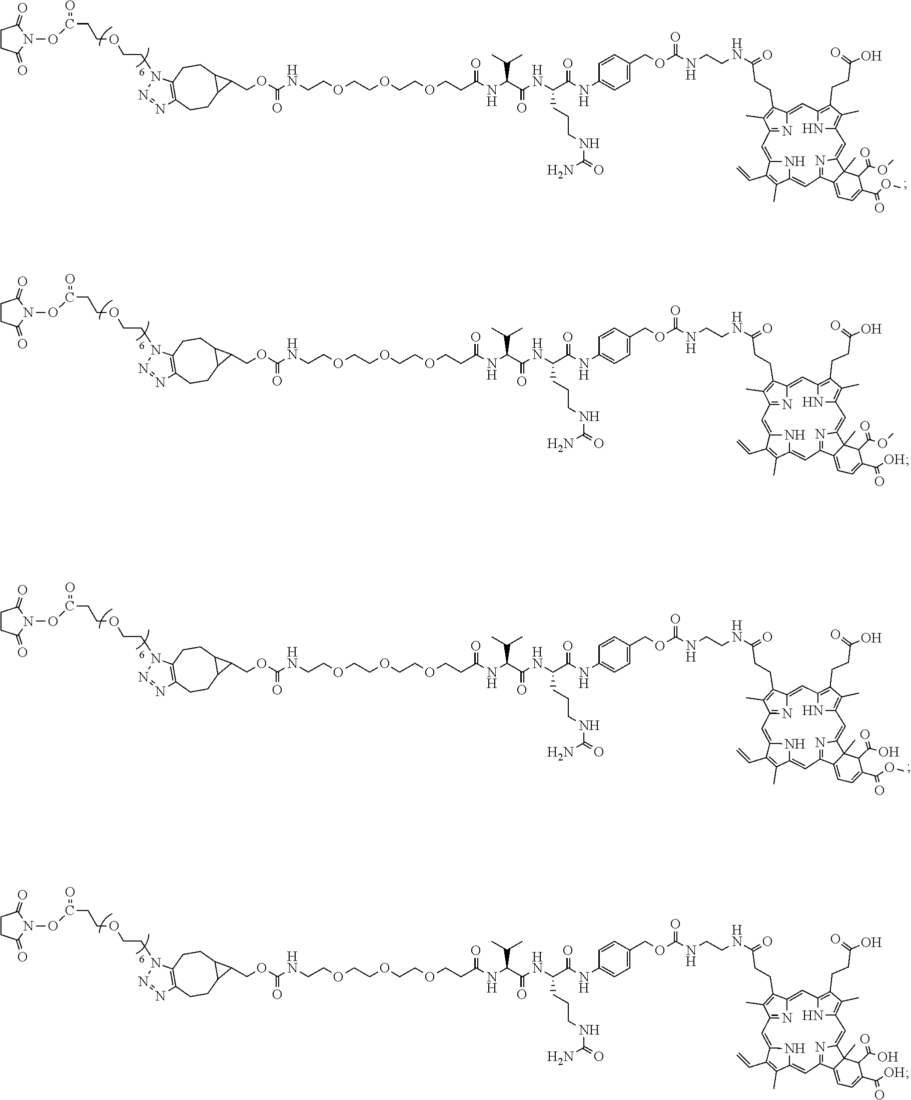

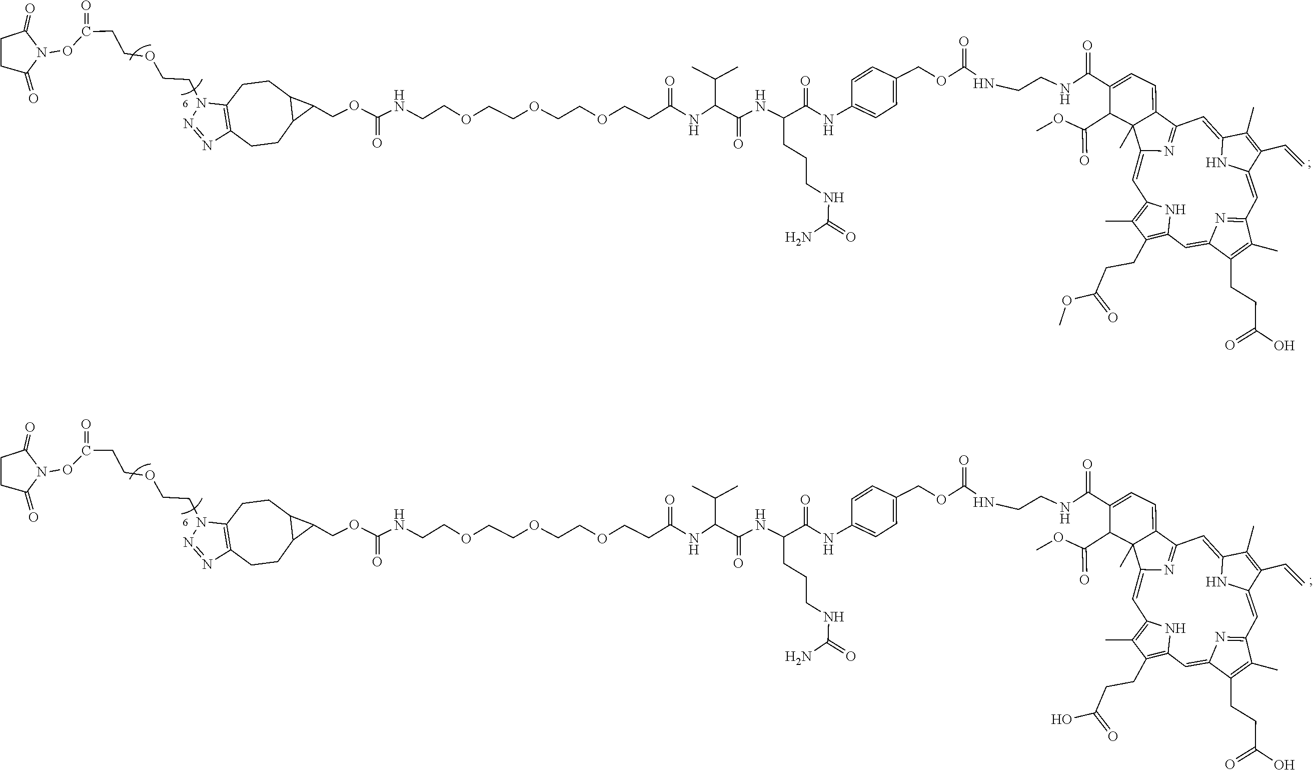

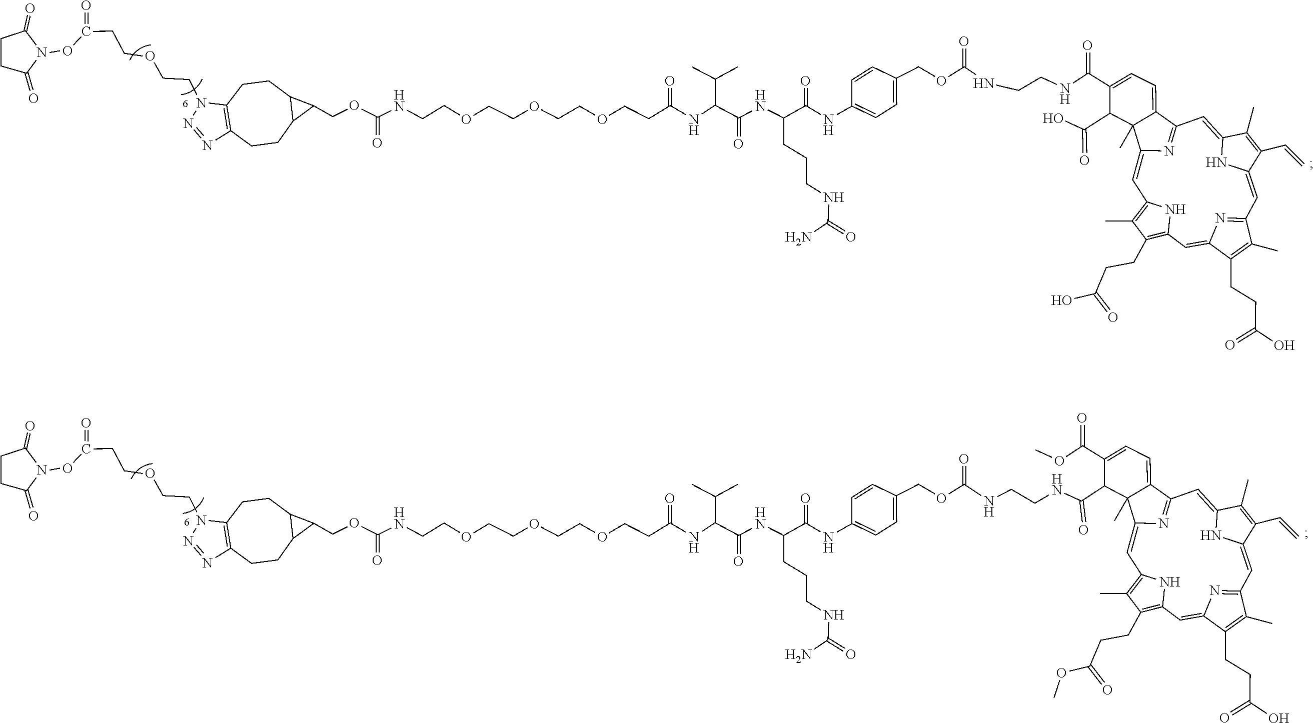

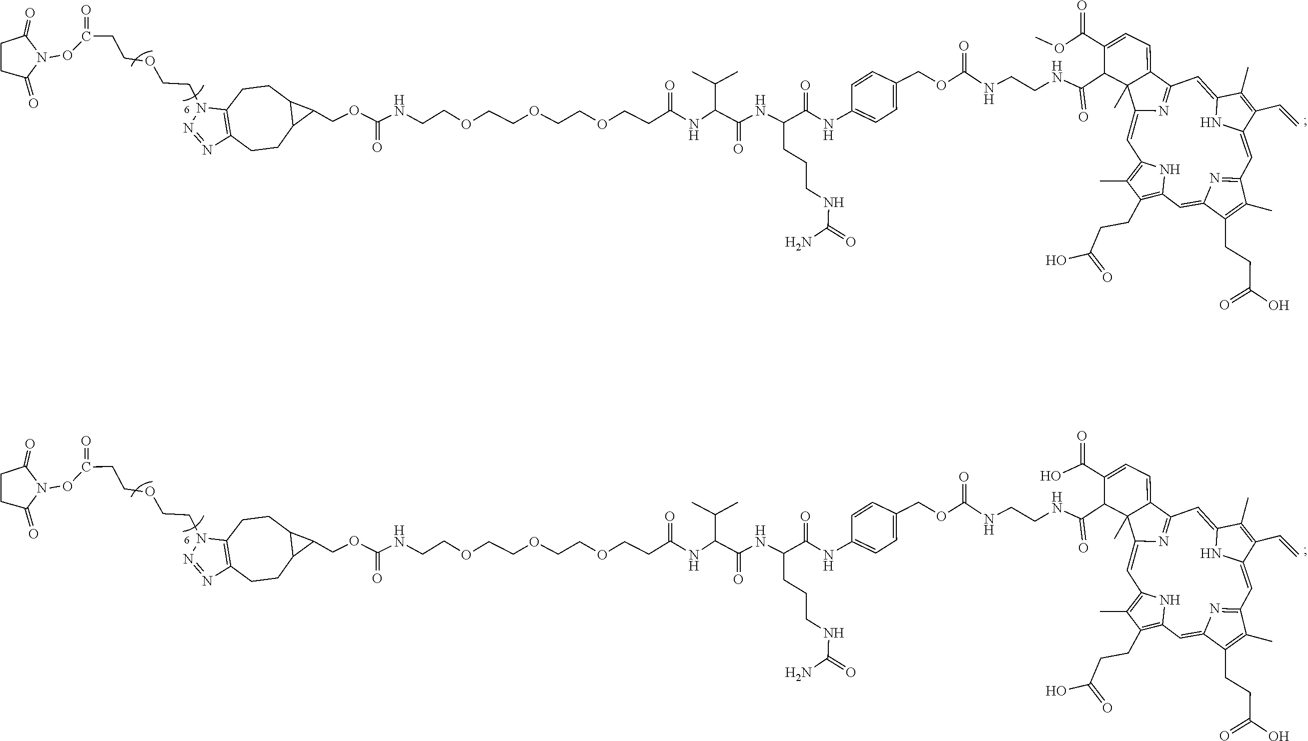

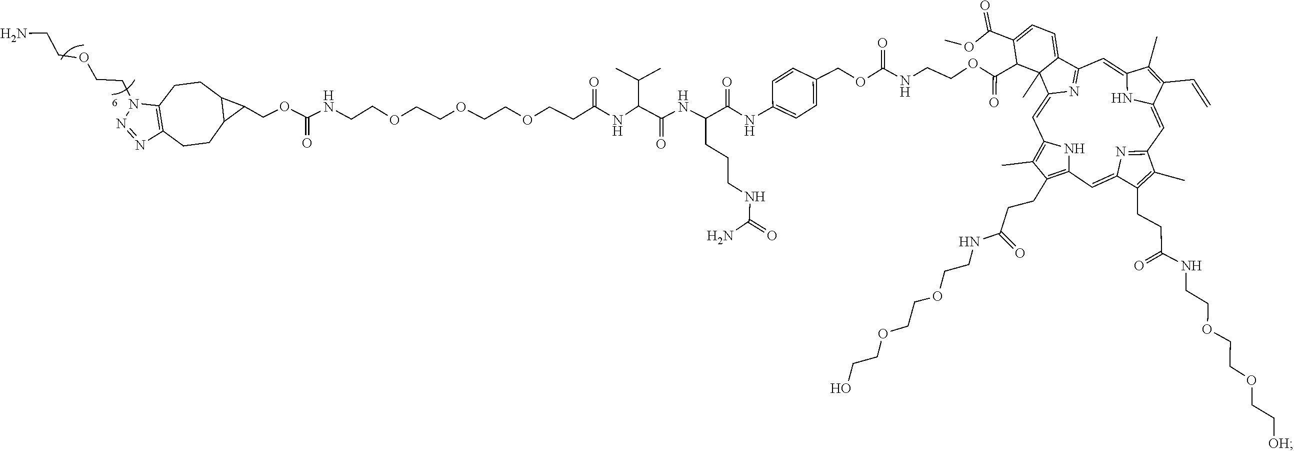

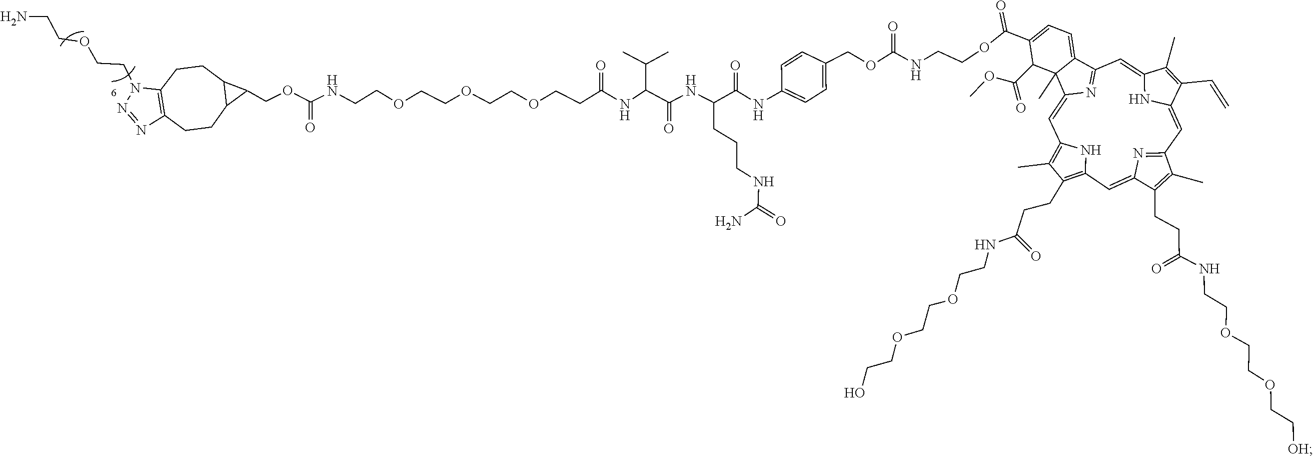

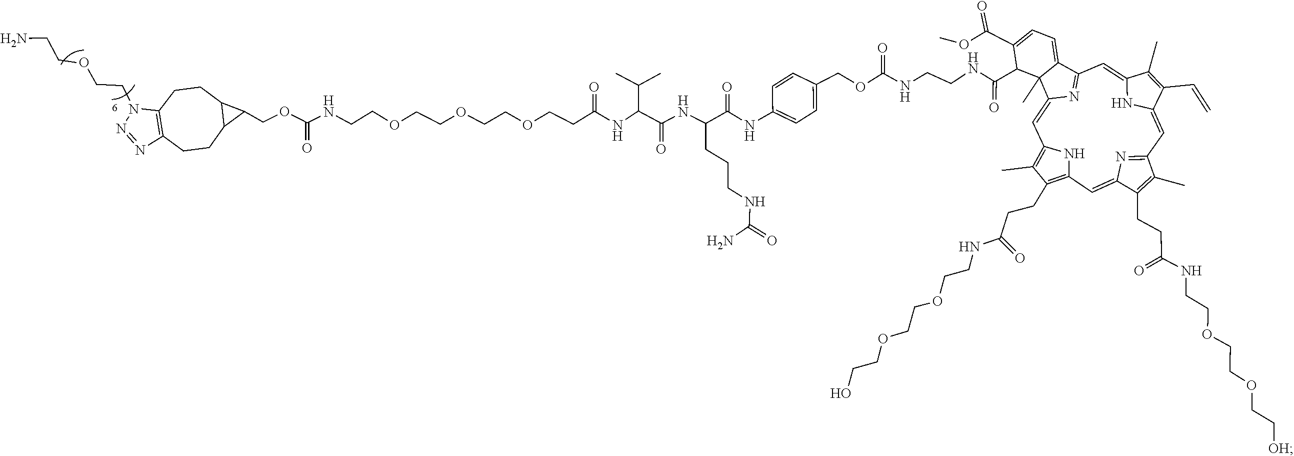

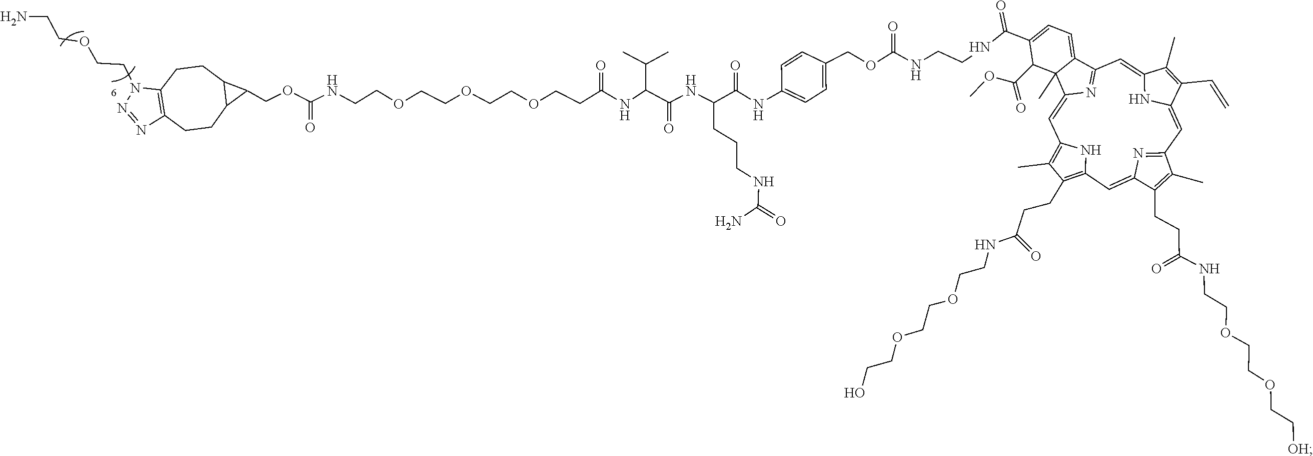

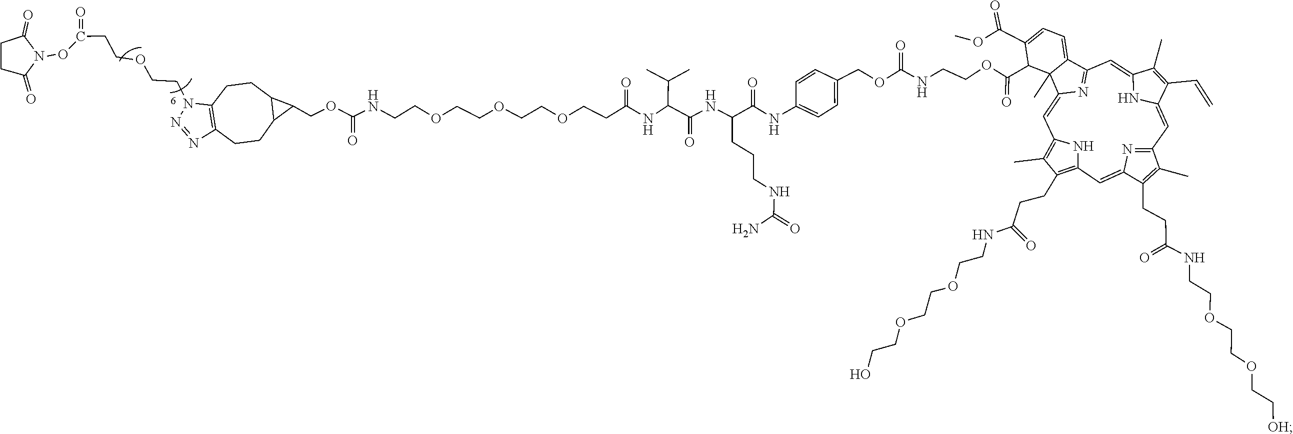

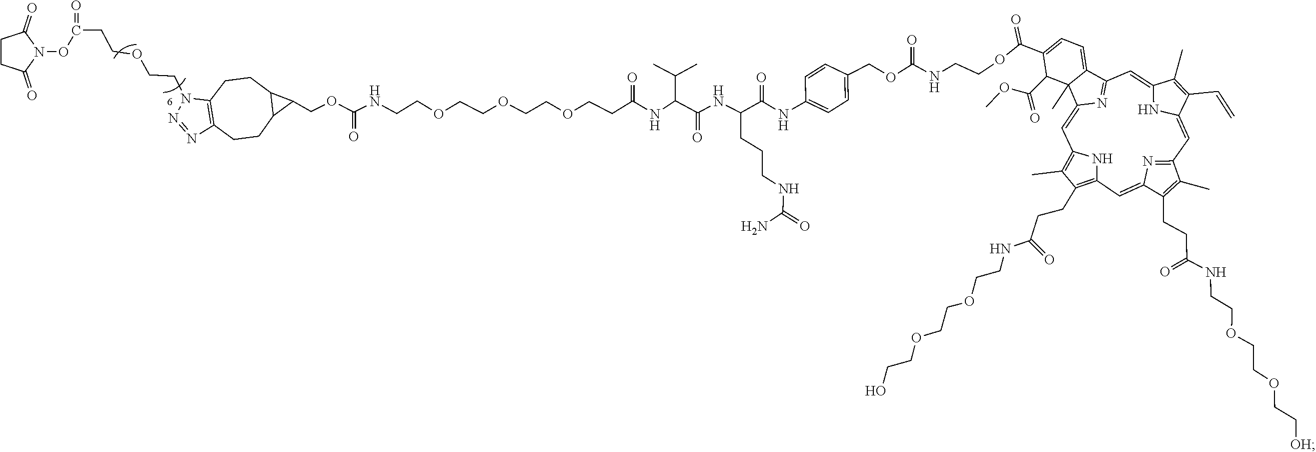

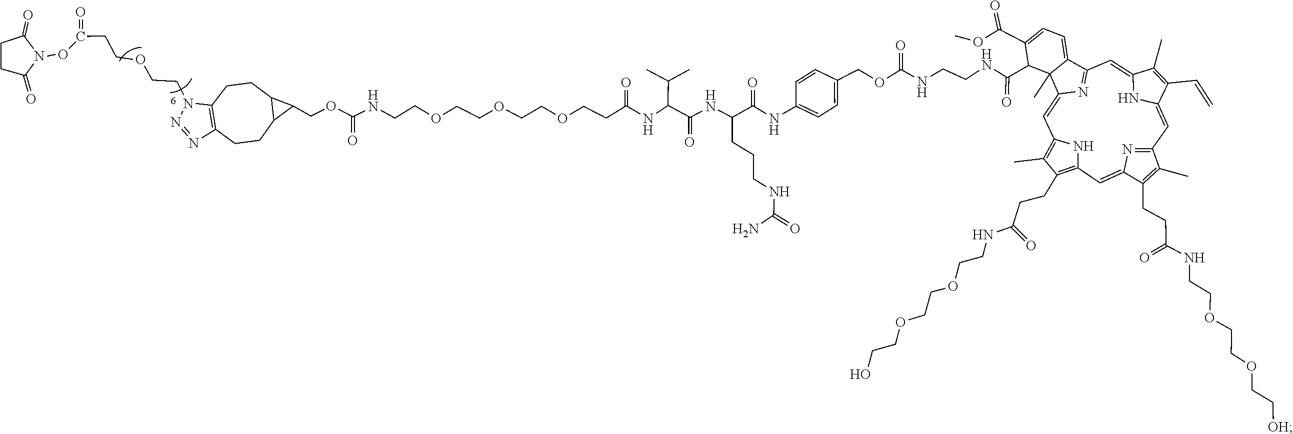

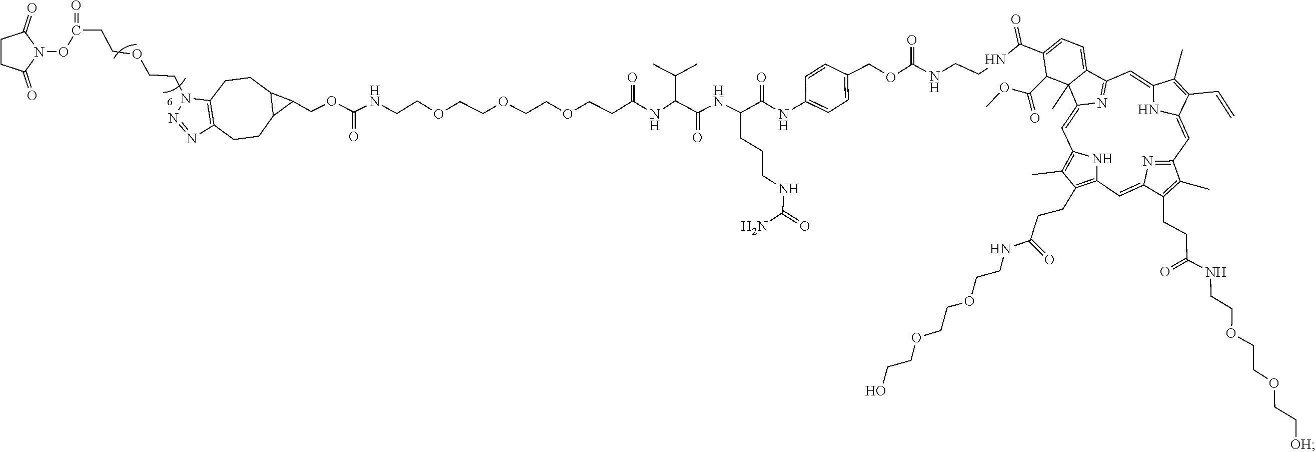

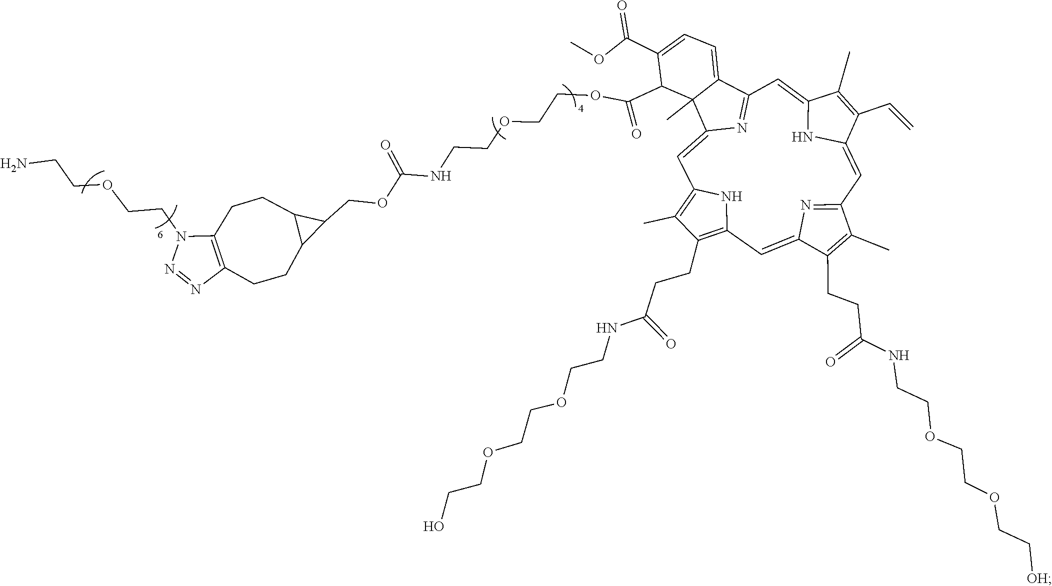

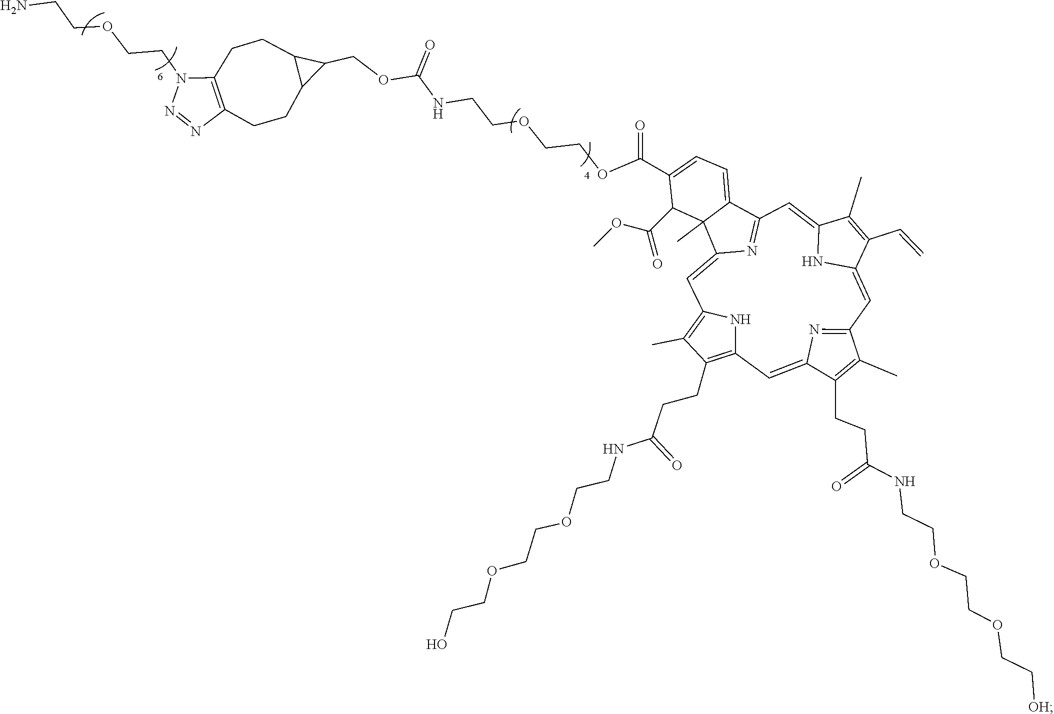

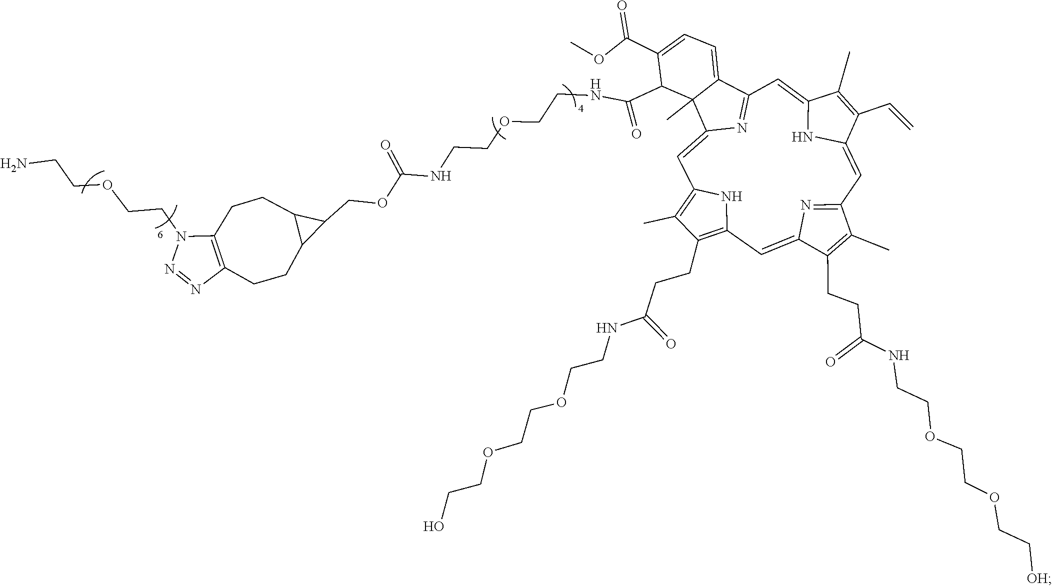

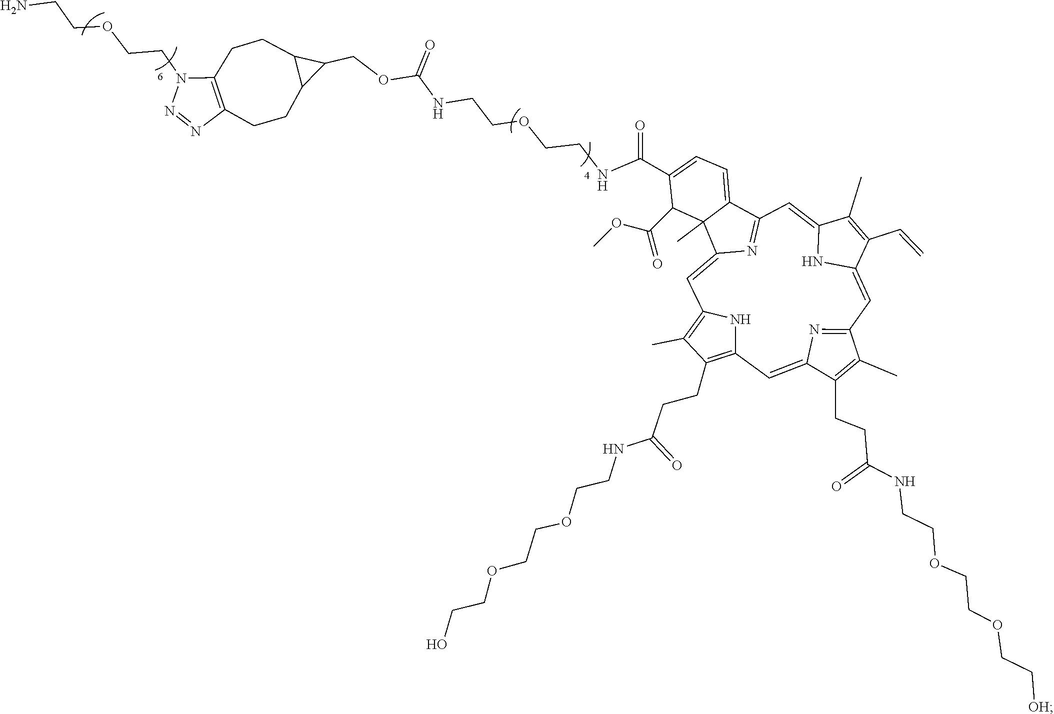

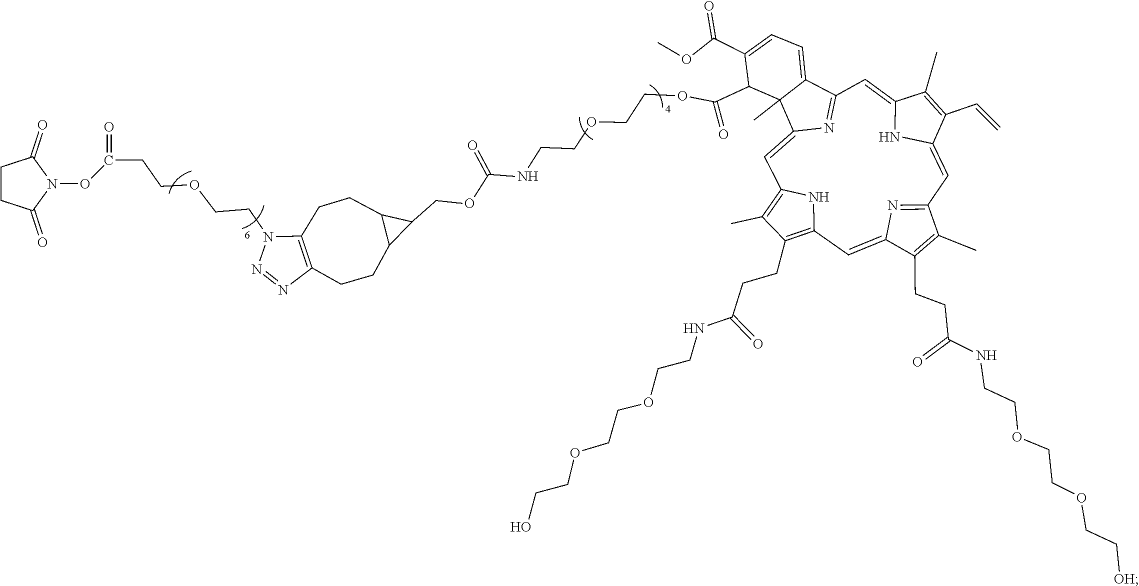

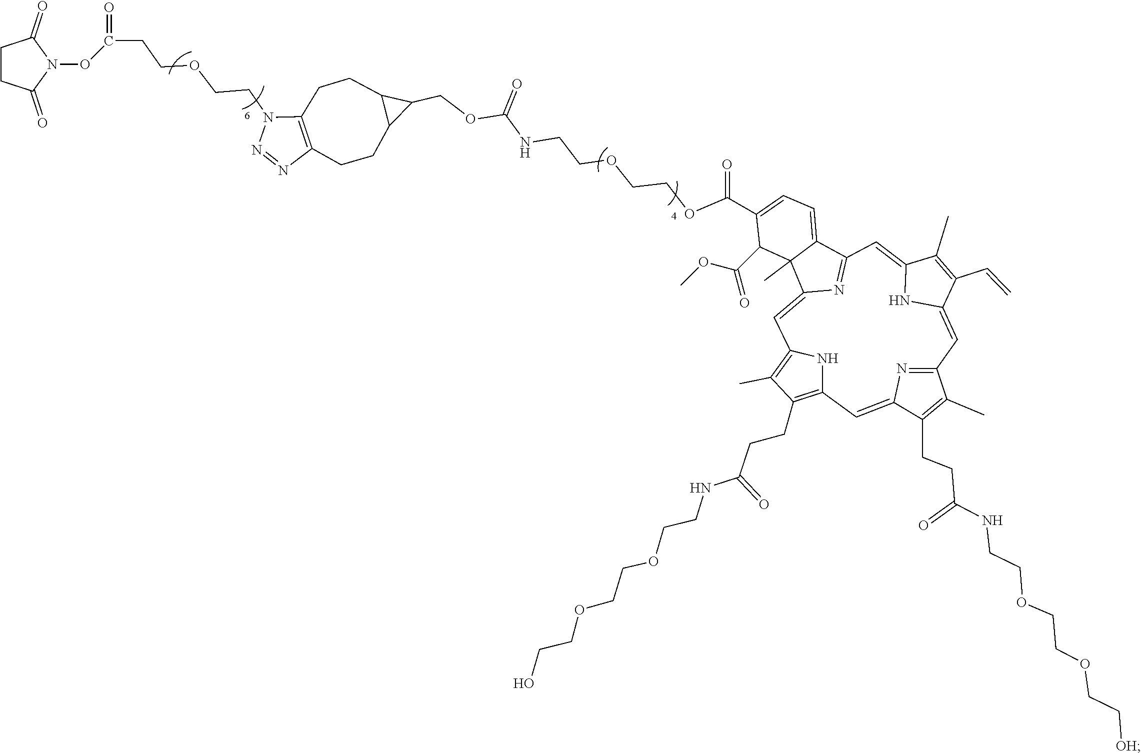

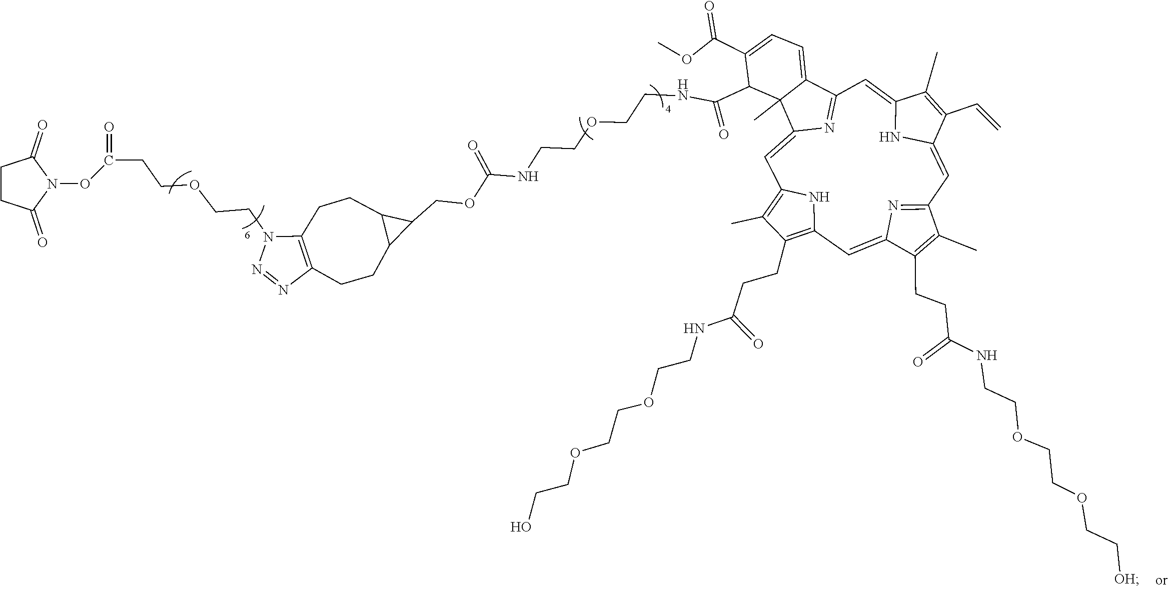

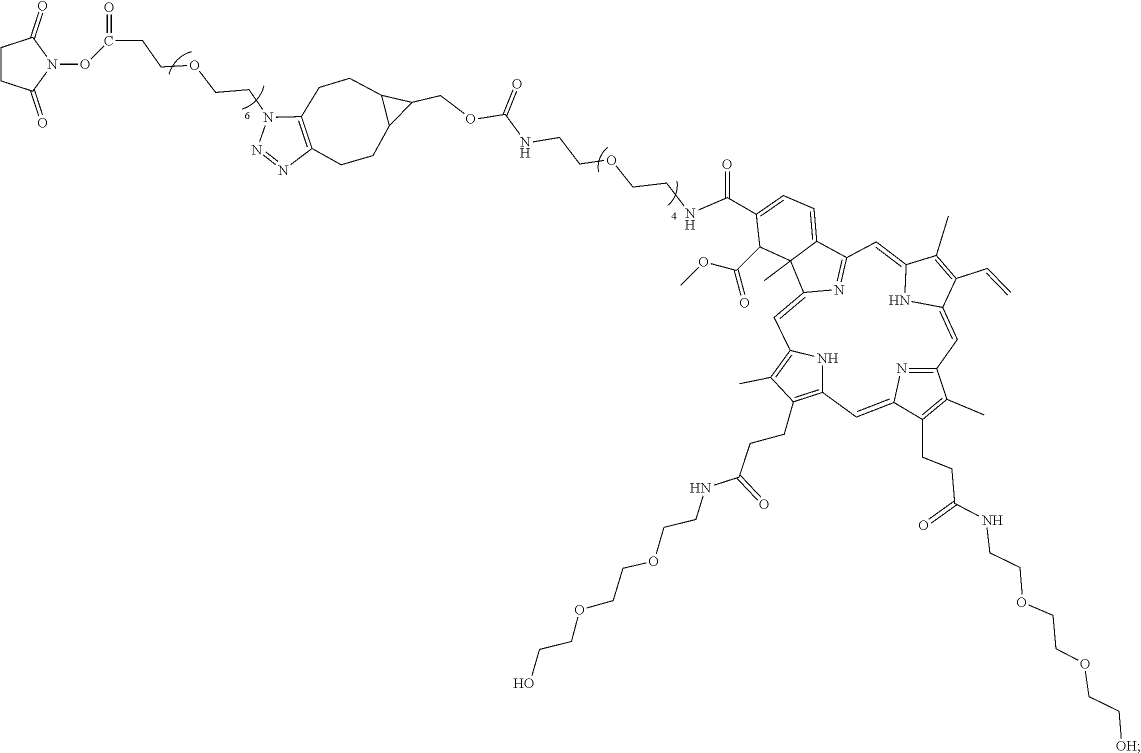

or a pharmaceutically acceptable salt thereof, a regioisomer, or a pharmaceutically acceptable salt of the regioisomer.

Embodiment 69. A conjugate comprising a benzoporphyrin analog covalently linked to a targeting agent, wherein the conjugate is represented by the following formula:

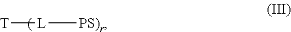

or a pharmaceutically acceptable salt thereof, wherein:
T is targeting agent;
L is a spacer connecting the targeting agent and PS;
r is an integer from 1 to 20;
PS is a photosensitizer that is a benzoporphyrin analog represented by the following formula:

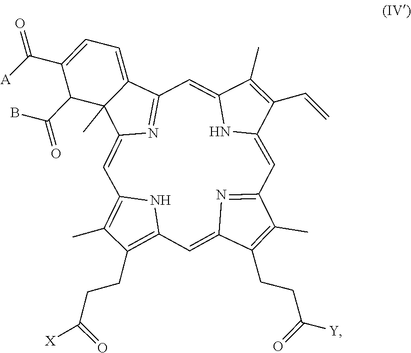

or a pharmaceutically acceptable salt thereof, wherein:
A, B, X and Y are each independently —OH, —OC₁₋₄alkyl, —N(R¹⁰⁰)(R¹⁰¹) or a bond covalently linked to L, provided one of A, B, X and Y is a bond covalently linked to L;
R¹⁰⁰ is H or C₁₋₃alkyl;
R¹⁰¹ is H, C₁₋₃alkyl or —(CH₂—CH₂—O)ₙᵣR¹⁰²;
R¹⁰² is H or Me;
nr is an integer from 1 to 16.

Embodiment 70. The conjugate of embodiment 69, or a pharmaceutically acceptable salt thereof, wherein -L-PS is represented by the following formula:

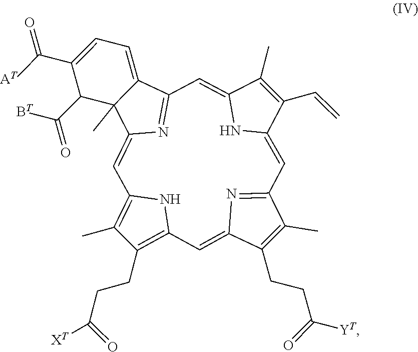

or a pharmaceutically acceptable salt thereof, wherein:
A^{T}, B^{T}, X^{T} and Y^{T} are each independently —OH, —OC₁₋₄alkyl, —N(R¹⁰⁰)(R¹⁰¹) or —Z-L^{P}-linker-L^{A}-R^{T}, provided one of A^{T}, B^{T}, X^{T} and Y^{T} is —Z-L^{P}-linker-L^{A}-R^{T}.

Z is —O— or —NR¹—;
R¹ is H or C₁₋₃alkyl;
L^{P} and L^{A} are each independently a spacer;
Linker is connection moiety formed by a first reactive group attached to L and a second reactive group attached to L^{A}; and
R^{T} is a reactive group covalently linked to the targeting agent.

Embodiment 71. The conjugate of embodiment 69 or 70, or a pharmaceutically acceptable salt thereof, wherein the targeting agent comprises a polypeptide that binds to the surface of a target cell.

Embodiment 72. The conjugate of embodiment 71, or a pharmaceutically acceptable salt thereof, wherein the targeting agent is an antibody or an antigen-binding fragment thereof.

Embodiment 73. The conjugate of embodiment 72, or a pharmaceutically acceptable salt thereof, wherein the antibody or antigen-binding fragment thereof binds to an adipose cell, blood cell, cancer cell, endothelial cell, epithelial cell, immune cell, neuron, skin cell, stem cell, tumor cell, myeloid derived suppressor cell.

Embodiment 74. The conjugate of embodiment 72, or a pharmaceutically acceptable salt thereof, wherein the antibody or antigen-binding fragment thereof binds to a cancer-associated fibroblast, a tumor-associated macrophage, a T cell, or a regulatory T cell.

Embodiment 75. The conjugate of embodiment 72, or a pharmaceutically acceptable salt thereof, wherein the antibody or antigen-binding fragment thereof binds to EGFR/HER1, HER2, HER3, HER4, VEGF, VEGFR, VEGFR2, EpCAM, E-Cad, Folate Receptor alpha, Fibroblast activation protein (FAP), CD1c, CD2, CD3, CD4, CD5, CD6, CD7, CD8, CD11a, CD11b, CD11c, CD14, CD16, CD18, CD19, CD20, CD21, CD25, CD26, CD27, CD28, CD30, CD31, CD32, CD33, CD44, CD45, CD52, CD56, CD62L, CD64, CD66b, CD69, CD80, CD86, CD90, CD103, CD122, CD123, CD127, CD163, CD206, CD235a, CXCR6, MHC-II, CCR4, CCR5, CCR7, CLA, PD-1, PD-L1, CTLA-4, , CEA, MUC-1, PSMA, Cancer antigen 125 (CA125), Alpha-fetoprotein (AFP), Lewis Y antigen, TAG-72, IL-13R, melanoma-associated antigen (MAGE)₁, MAGE2, MAGE3, MAGE4, tumor-associated glycoprotein 72 (TAG-72), gp100, p97 melanoma antigen, human milk fat globule (HMFG), melanoma antigen recognized by T cells 1 (MART1), B melanoma antigen (BAGE) 1, BAGE2, G antigen (GAGE) 1, GAGE2, GAGE3, GAGE4, GAGE5, GAGE6, breast cancer-associated DF3 antigen, New York esophageal squamous cell carcinoma 1 (NY-ESO-1), mesothelin, GITR, OX40, FR4, CXCR4, CCL4, Gr-1, IL-4Ra, IL-1Ra, CXCR2, or LAG-3.

Embodiment 76. The conjugate of embodiment 75, or a pharmaceutically acceptable salt thereof, wherein the antibody or antigen-binding fragment thereof binds to CD2 or EGFR.

Embodiment 77. The conjugate of any one of embodiments 70-76, or a pharmaceutically acceptable salt thereof, wherein the targeting agent comprises a reactive group that can form a covalent bond with R^{T}.

Embodiment 78. The conjugate of embodiment 77, or a pharmaceutically acceptable salt thereof, wherein the targeting agent comprises a reactive group selected from an amine group, a thiol group or an amide group.

Embodiment 79. The conjugate of embodiment 77, or a pharmaceutically acceptable salt thereof, wherein the targeting agent comprises the following group:

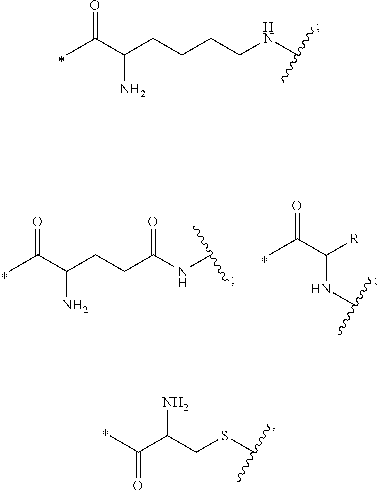

wherein R is side chain of an amino acid residue; *- represents a bond to the remaining targeting agent and

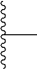

represents a bond to R^{T}.

Embodiment 80. The conjugate of any one of embodiments 70-79, or a pharmaceutically acceptable salt thereof, wherein R^{T} is —C(═O)—, —NH—, or

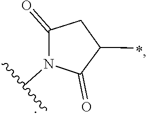 [SMILES: *N1C(=O)CC(C)C1=O]

wherein *- represents a bond to the targeting agent and

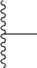

represents a bond to L^{A}.

Embodiment 81. The conjugate of embodiment 78, or a pharmaceutically acceptable salt thereof, wherein R^{T} is —C(═O)— or —NH—.

Embodiment 82. The conjugate of any one of embodiments 70-81, or a pharmaceutically acceptable salt thereof, wherein L^{A} is a spacer comprising a hydrophilic moiety.

Embodiment 83. The conjugate of embodiment 82, or a pharmaceutically acceptable salt thereof, wherein L^{A} is a spacer comprising a PEG moiety.

Embodiment 84. The conjugate of embodiment 82, or a pharmaceutically acceptable salt thereof, wherein L^{A} is *—CH₂CH₂(OCH₂CH₂)ₚ—, wherein * is the site connected to R^{T}; and p is an integer from 0 to 30.

Embodiment 85. The conjugate of embodiment 84, or a pharmaceutically acceptable salt thereof, wherein p is an integer from 2 to 16, from 2 to 10, from 2 to 8, or from 3 to 5; or p is 2, 3, 4, 5, 6, 7 or 8.

Embodiment 86. The conjugate of any one of embodiments 70-85, or a pharmaceutically acceptable salt thereof, wherein the first reactive group and the second reactive group are each selected from a maleimide group, a thiol group, a cyclooctyne group, and an azido group.

Embodiment 87. The conjugate of embodiment 86, or a pharmaceutically acceptable salt thereof, wherein Linker is represented by the following formula:

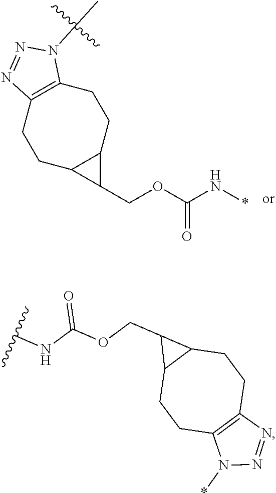 [SMILES: *NC(=O)OCC1C2CCc3nnn(*)c3CCC21 ; *NC(=O)OCC1C2CCc3nnn(*)c3CCC21]

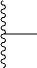

wherein *- represents a bond to L^{P} and represents a bond to L^{A}.

Embodiment 88. The conjugate of any one of embodiments 70-87, or a pharmaceutically acceptable salt thereof, wherein:
a) A^{T} and B^{T} are —OCH₃; and one of X^{T} and Y^{T} is —OCH₃ and the other one of X^{T} and Y^{T} is —Z-L^{P}-linker-L^{A}-R^{T};
b) A^{T} and B^{T} are —OCH₃; and one of X^{T} and Y^{T} is —OH and the other one of X^{T} and YT is —Z-L^{P}-linker-L^{A}-R^{T}.
c) one of A^{T} and B^{T} is —OCH₃, and the other one of A^{T} and B^{T} is —OH; and one of X^{T} and Y^{T} is —OH and the other one of X^{T} and Y^{T} is —Z-L^{P}-linker-L^{A}-R^{T};
d) A^{T} and B^{T} are —OH; and one of X and Y is —OH and the other one of X^{T} and Y^{T} is —Z-L^{P}-linker-L^{A}-R^{T}; or
e) one of A^{T} and B^{T} is —Z-L^{P}-linker-L^{A}-R^{T} and the other one of A^{T} and B^{T} is —OH or —OCH₃; and X^{T} and Y^{T} are both —NH(CH₂—CH₂—O)ₙᵣR¹⁰², wherein R¹⁰² is H or Me; and nr is an integer from 2 to 8.

Embodiment 89. The conjugate of embodiment 88, or a pharmaceutically acceptable salt thereof, wherein -L-PS is represented by the following formula:

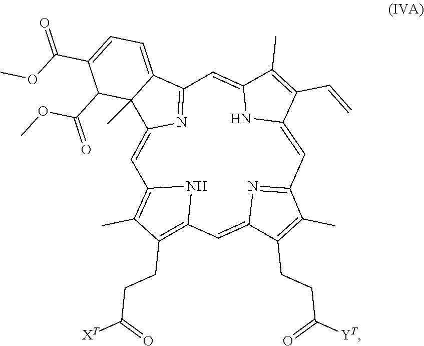

or a pharmaceutically acceptable salt thereof, wherein one of X^{T} and Y^{T} is —Z-L^{P}-linker-L^{A}-R^{T} and the other is —OH.

Embodiment 90. The conjugate of embodiment 88, or a pharmaceutically acceptable salt thereof, wherein -L-PS is represented by the following formula:

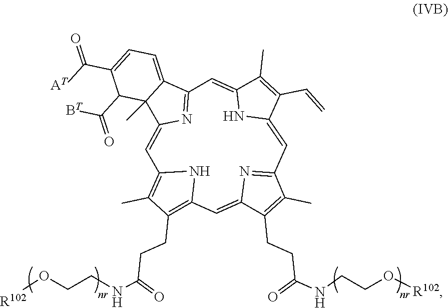

or a pharmaceutically acceptable salt thereof, wherein one of A^{T} and B^{T} is —Z-L^{P}-linker-L^{A}-R^{T} and the other is —OH or —OCH₃; R¹⁰² is H or Me; and nr is an integer from 2 to 8.

Embodiment 91. The conjugate of any one of embodiments 70-90, or a pharmaceutically acceptable salt thereof, wherein Z is —NH—.

Embodiment 92. The conjugate of any one of embodiments 70-90, or a pharmaceutically acceptable salt thereof, wherein Z is —O—.

Embodiment 93. The conjugate of any one of embodiments 70-92, or a pharmaceutically acceptable salt thereof, wherein L^{P} is a non-cleavable spacer.

Embodiment 94. The conjugate of embodiment 93, or a pharmaceutically acceptable salt thereof, wherein L^{P} comprises a hydrophilic moiety.

Embodiment 95. The conjugate of embodiment 94, or a pharmaceutically acceptable salt thereof, wherein L^{P} comprises a PEG moiety.

Embodiment 96. The conjugate of embodiment 95, or a pharmaceutically acceptable salt thereof, wherein L^{P} is *—CH₂CH₂—(OCH₂CH₂)ₘ—, wherein * is the sited connected to the Linker group, and m is an integer from 0 to 30.

Embodiment 97. The conjugate of embodiment 96, or a pharmaceutically acceptable salt thereof, m is an integer from 2 to 16, from 2 to 10, from 2 to 8, or from 3 to 5; or m is 2, 3, 4, 5, 6, 7 or 8.

Embodiment 98. The conjugate of embodiment 96, or a pharmaceutically acceptable salt thereof, or a pharmaceutically acceptable salt thereof, wherein L^{P} is*—CH₂CH₂—(OCH₂CH₂)₄—, wherein * is the sited connected to the Linker group.

Embodiment 99. The conjugate of any one of embodiments 70-98, or a pharmaceutically acceptable salt thereof, wherein L^{P} is a cleavable spacer.

Embodiment 100. The conjugate of embodiment 99, or a pharmaceutically acceptable salt thereof, wherein the cleavable spacer comprises an enzyme labile group, a pH labile group, a disulfide group, a ROS cleavable group, a photocleavable group.

Embodiment 101. The conjugate of embodiment 100, or a pharmaceutically acceptable salt thereof, wherein the enzyme labile group is a group cleavable by an esterase, a peptidase, an aminopeptidase, a β-galactosidase, a β-glucuronidase, a carboxylesterase, a caspase, a diaphorase, a histone deacetylase, a legumain, or matrix metalloproteinase.

Embodiment 102. The conjugate of embodiment 100, or a pharmaceutically acceptable salt thereof, wherein the enzyme labile group is a group cleavable by cathepsin B.

Embodiment 103. The conjugate of embodiment 99, or a pharmaceutically acceptable salt thereof, wherein the spacer represented by L^{P} comprises a peptide and a self-immolative group.

Embodiment 104. The conjugate of embodiment 103, or a pharmaceutically acceptable salt thereof, wherein the spacer L^{P} is represented by the following formula:

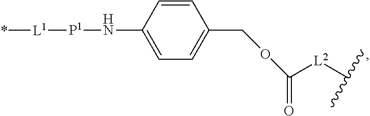

wherein:
*- represents a bond to the group represented by Linker;

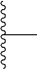

represents a bond to the group Z;
L¹ and L² are each independently a connecting spacer;
P¹ is a peptide comprising 2 to 5 amino acid residues.

Embodiment 105. The conjugate of embodiment 104, or a pharmaceutically acceptable salt thereof, wherein P¹ is a peptide selected from Phe-Arg-Arg-Gly, Glu-Val-Cit, Val-Cit, Cit-Val, Gly-Gly-Phe, Val-Ala, and Ala-Val.

Embodiment 106. The conjugate of embodiment 104, or a pharmaceutically acceptable salt thereof, wherein P¹ is:

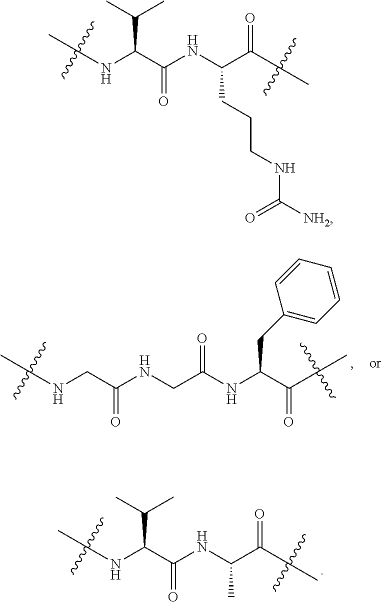

Embodiment 107. The conjugate of any one of embodiments 104-106, or a pharmaceutically acceptable salt thereof, wherein L¹ comprises a hydrophilic moiety.

Embodiment 108. The conjugate of embodiment 107, or a pharmaceutically acceptable salt thereof, wherein L¹ comprises a PEG moiety.

Embodiment 109. The conjugate of embodiment 107, or a pharmaceutically acceptable salt thereof, wherein L¹ is *—CH₂—CH₂—(OCH₂CH₂)ₙ—C(═O)—**; n is an integer from 0 to 30; *- represents a bond to the Linker group; **- represents a bond to P¹.

Embodiment 110. The conjugate of embodiment 109, or a pharmaceutically acceptable salt thereof, wherein n is an integer from 2 to 16, from 2 to 10, from 2 to 8, or from 3 to 5; or n is 2, 3, 4, 5, 6, 7 or 8.

Embodiment 111. The conjugate of embodiment 107, or a pharmaceutically acceptable salt thereof, wherein L¹ is *—CH₂—CH₂—(OCH₂CH₂)₃—C(═O)—**.

Embodiment 112. The conjugate of any one of embodiments 104-111 or a pharmaceutically acceptable salt thereof, wherein L² is —NH—C₁₋₆alkyl-**, wherein ** is the site connected to Z.

Embodiment 113. The conjugate of embodiment 112, or a pharmaceutically acceptable salt thereof, wherein L² is —NH—CH₂CH₂—**, wherein ** is the site connected to Z.

Embodiment 114. The conjugate of embodiment 69, or a pharmaceutically acceptable salt thereof, wherein -L-PS is represented by the following formula:

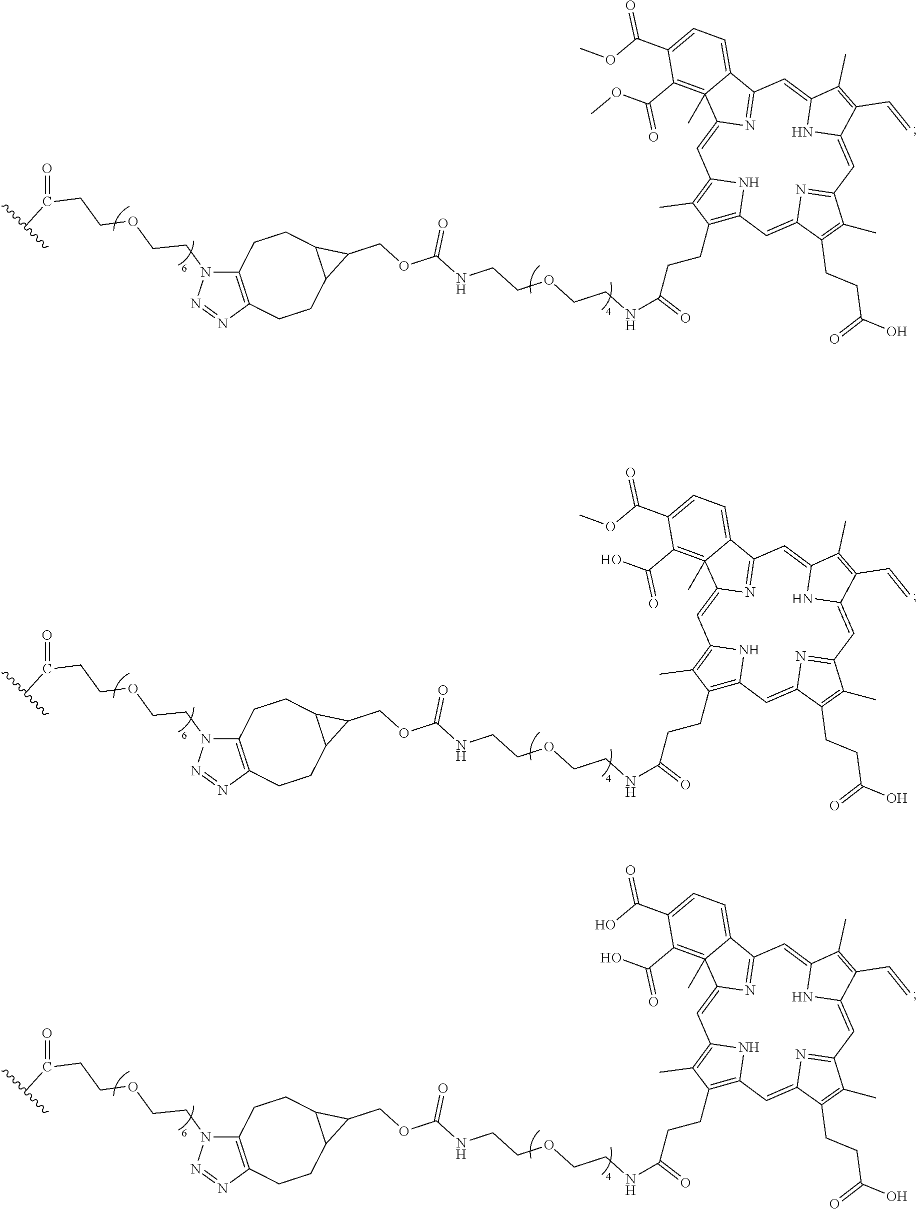

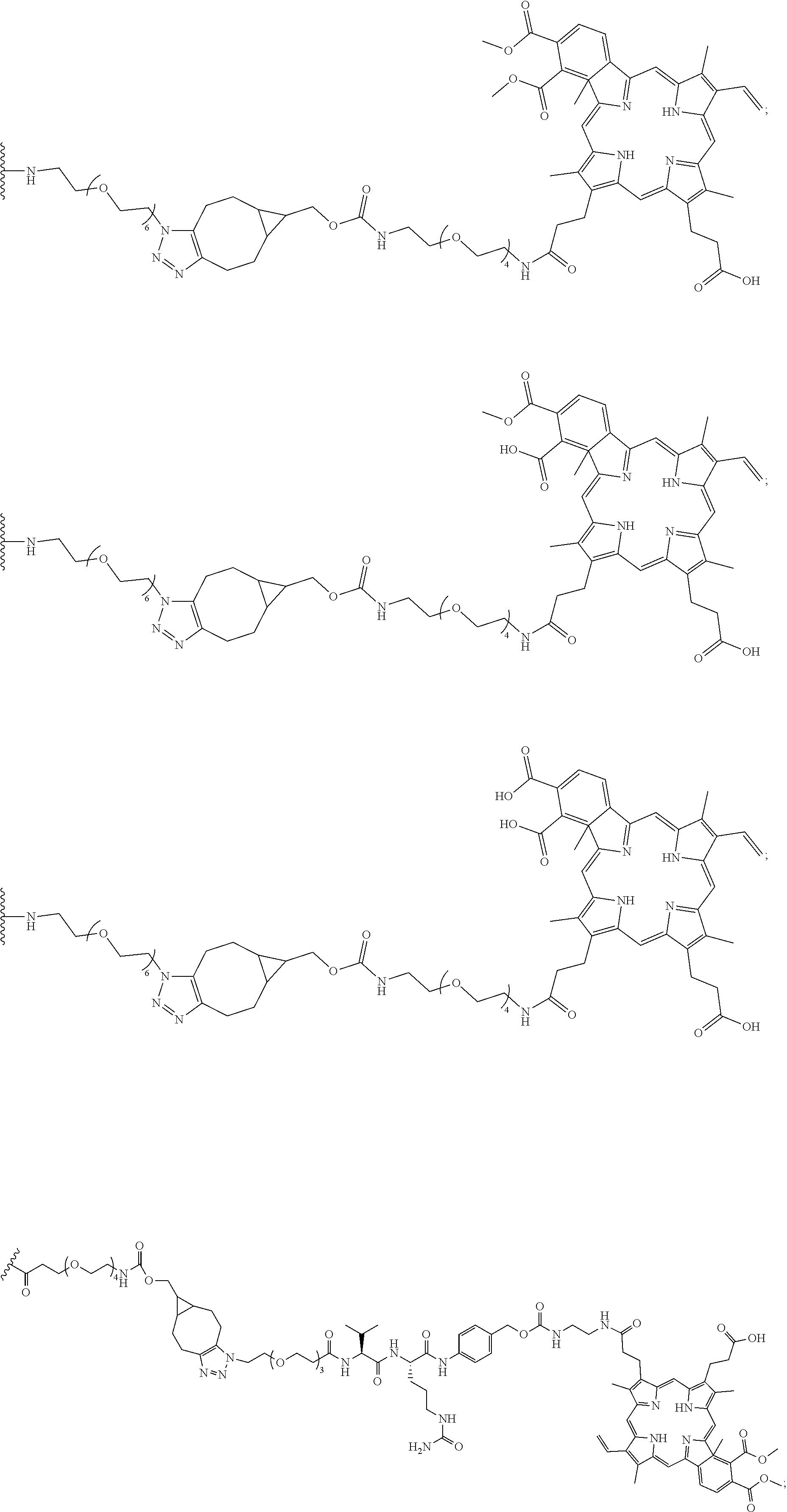

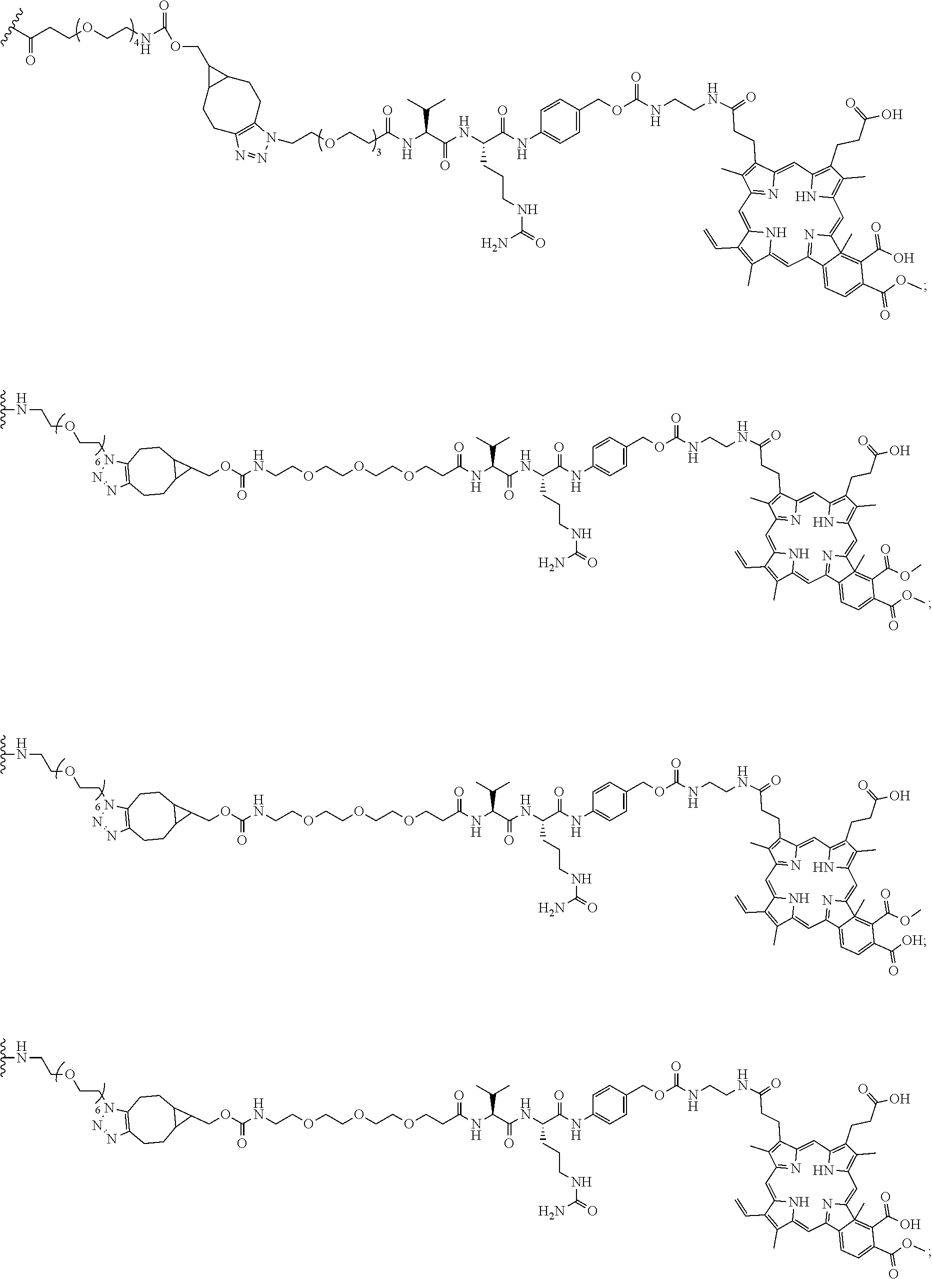

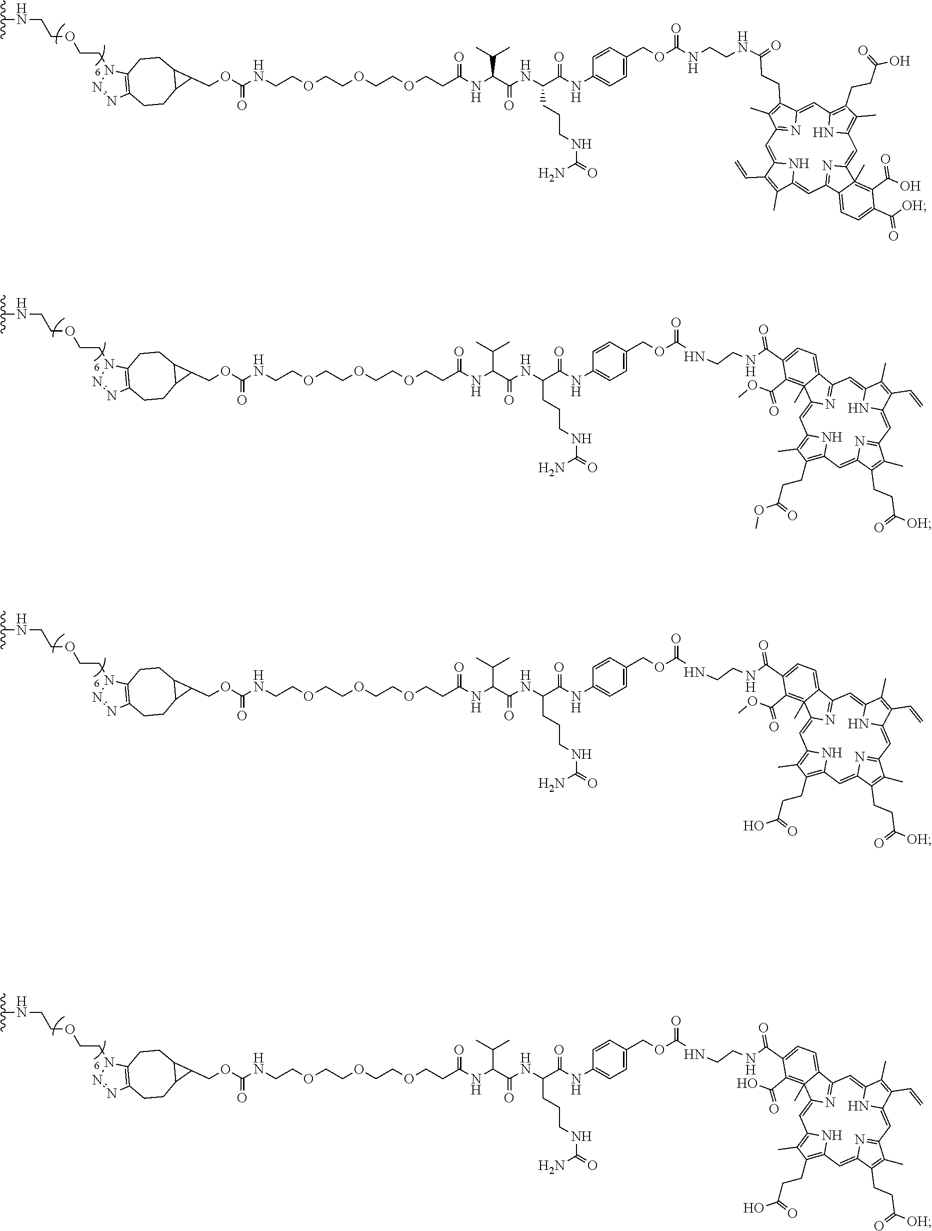

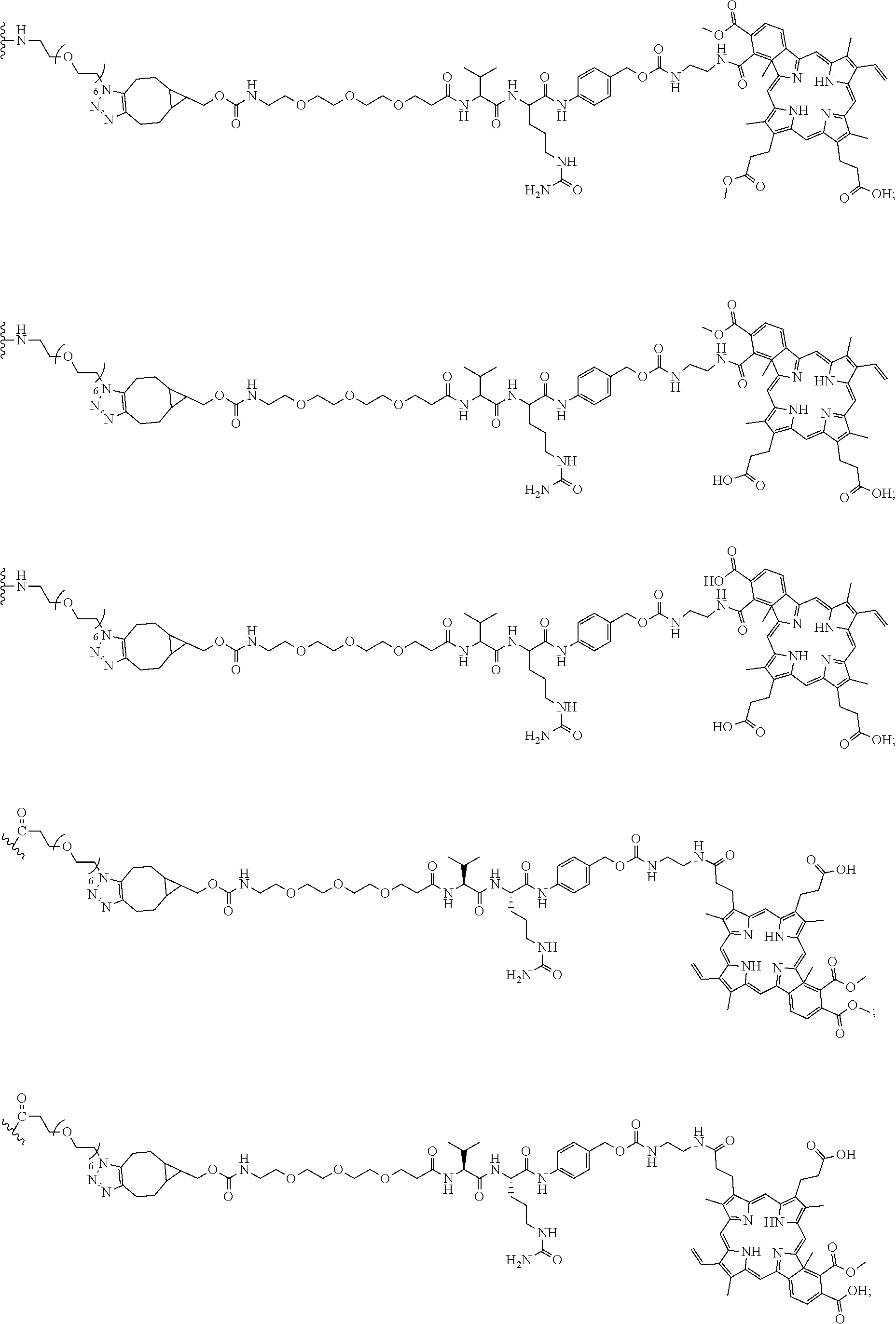

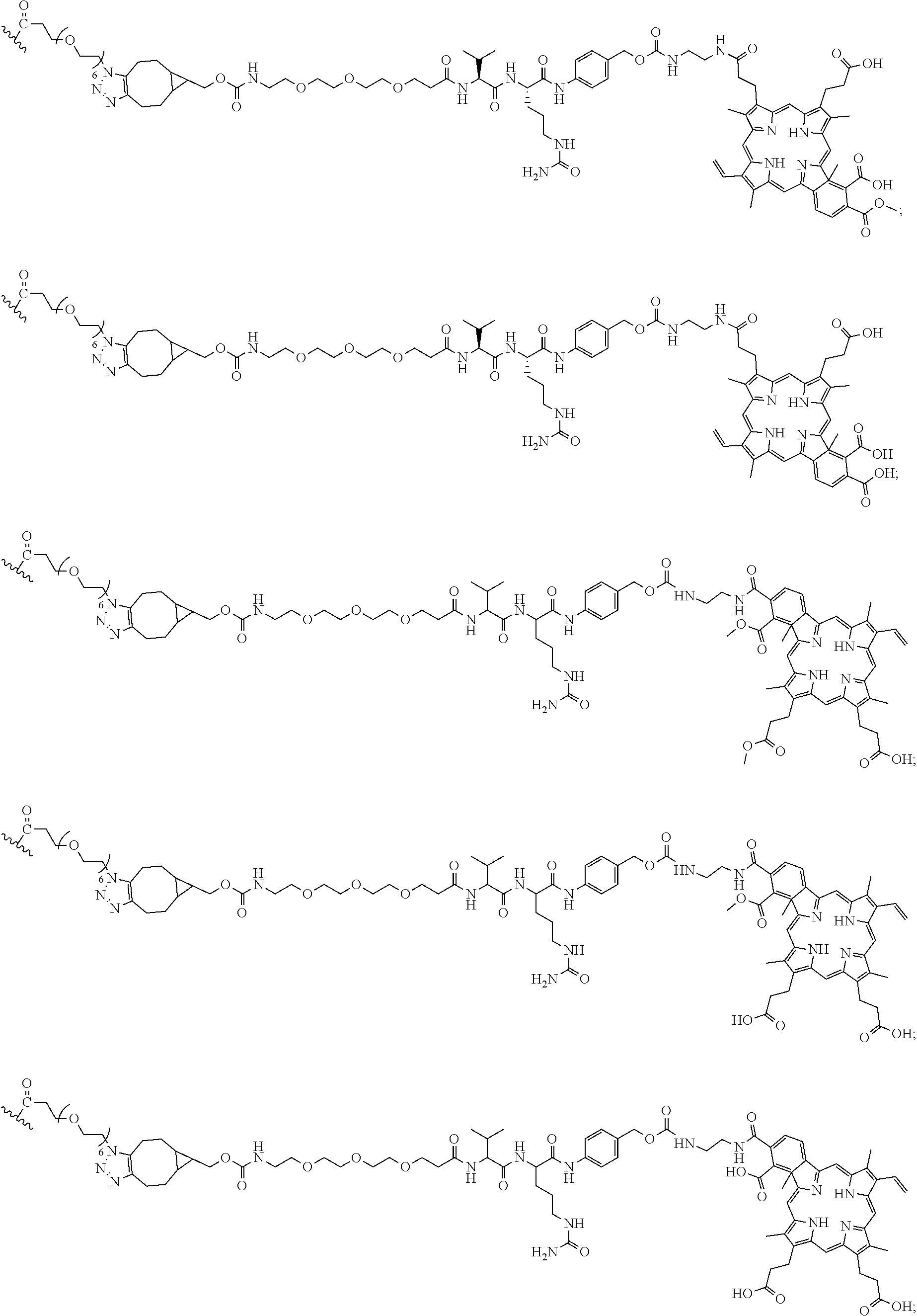

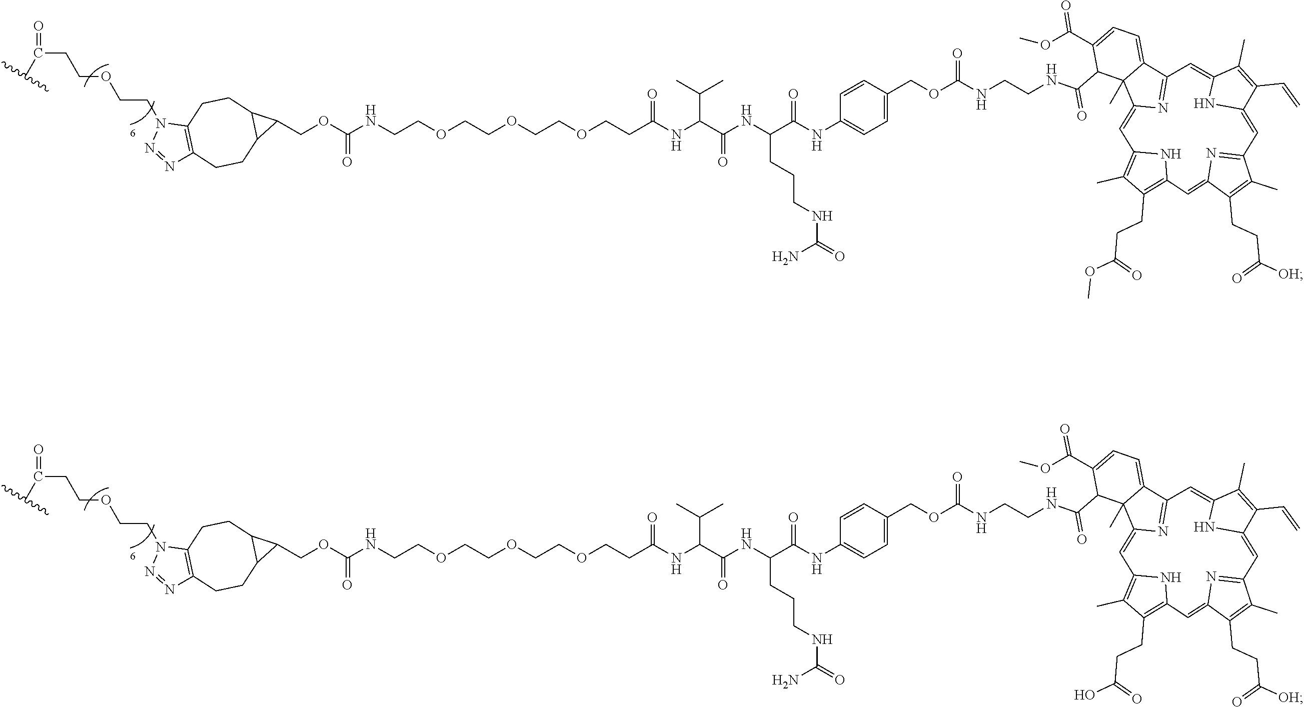

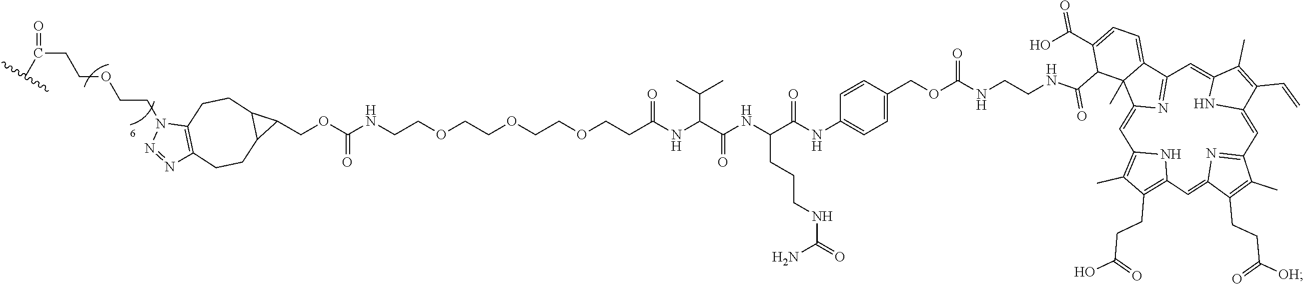

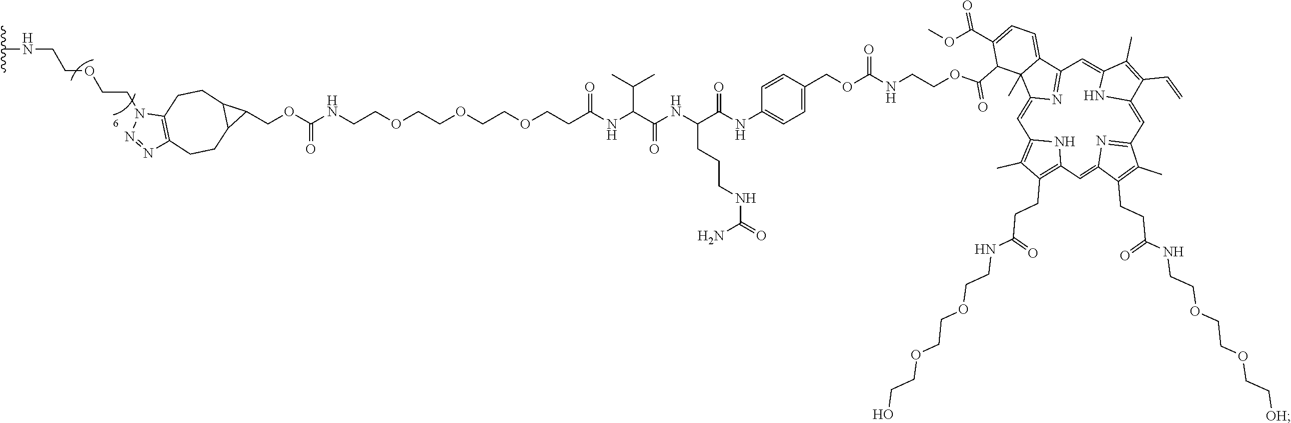

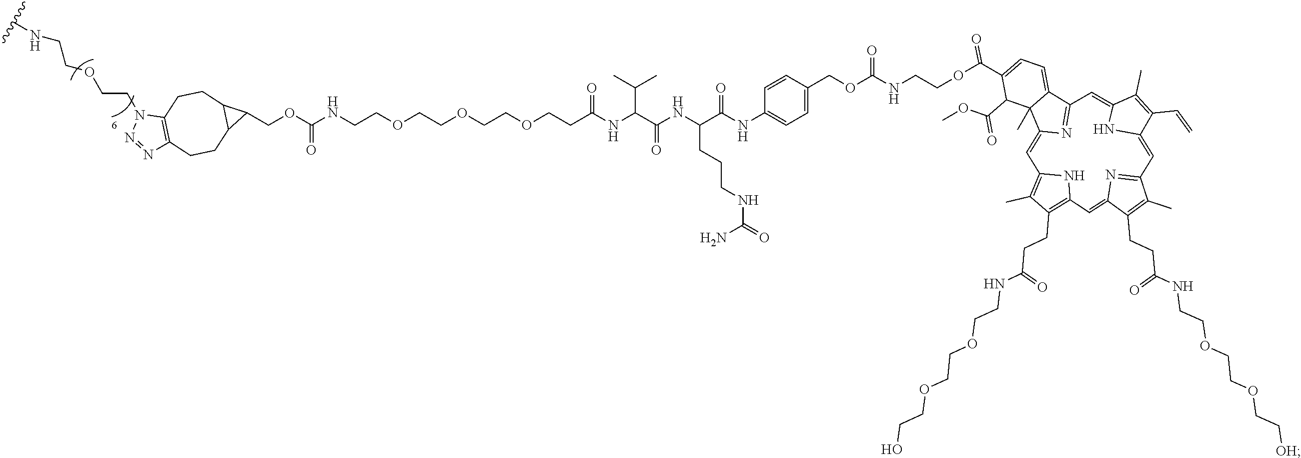

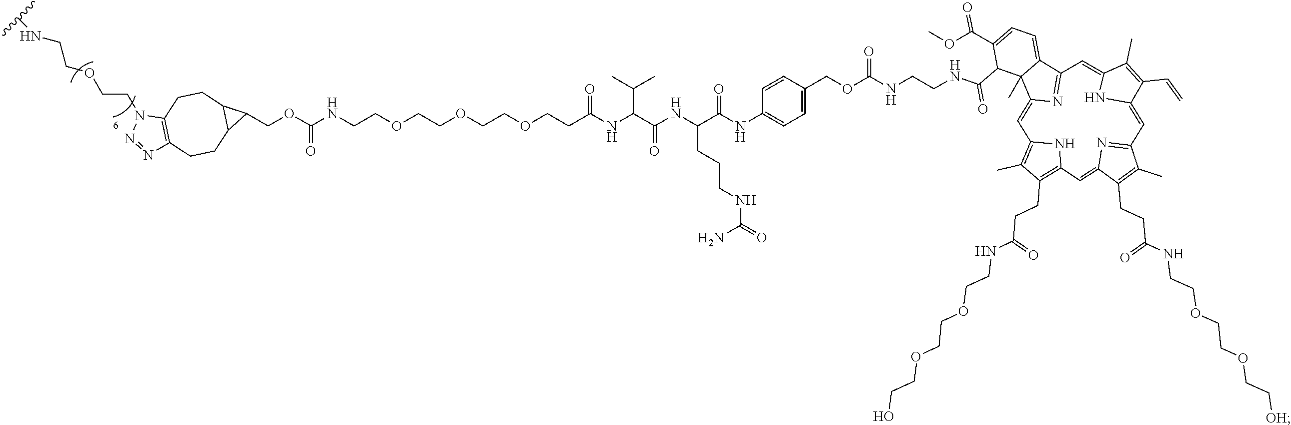

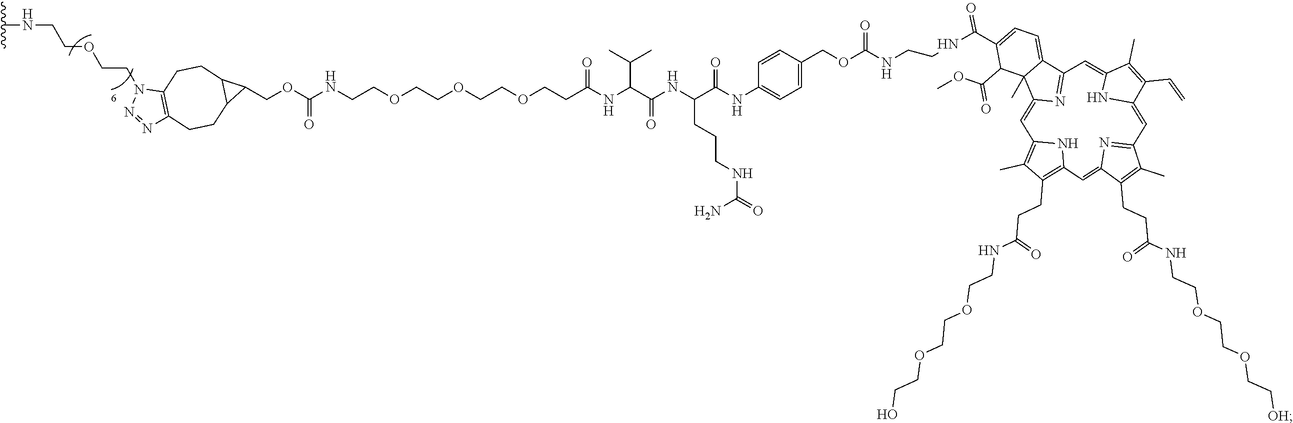

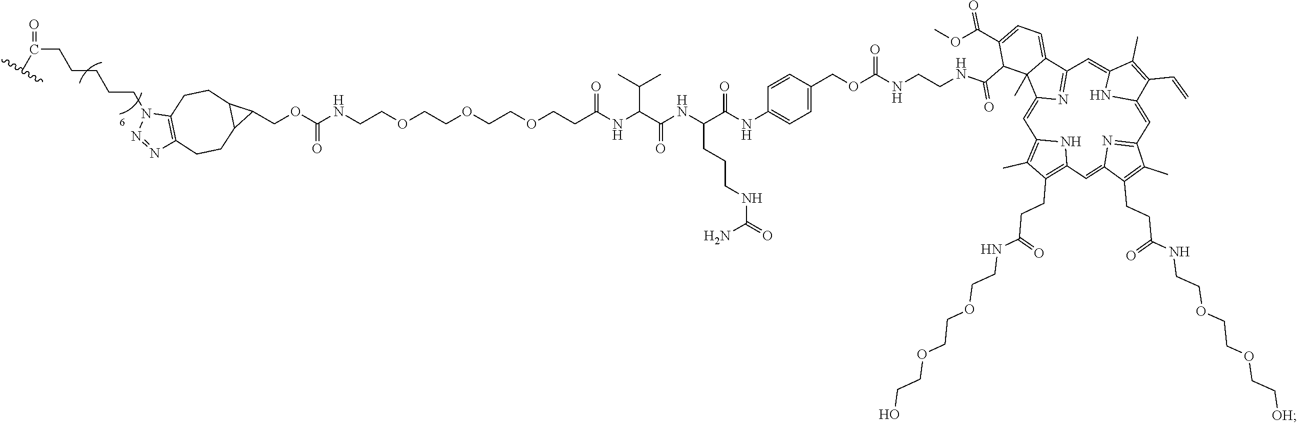

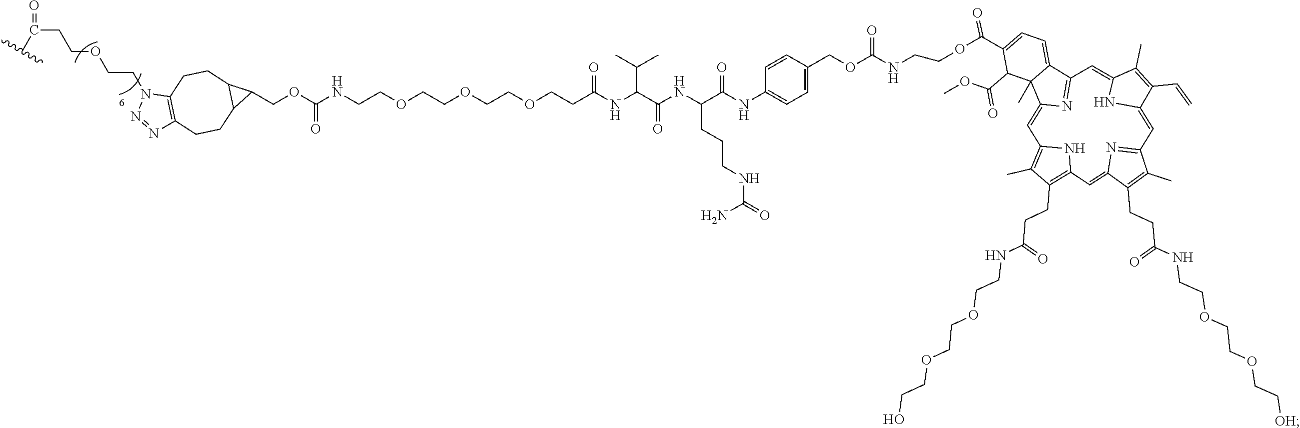

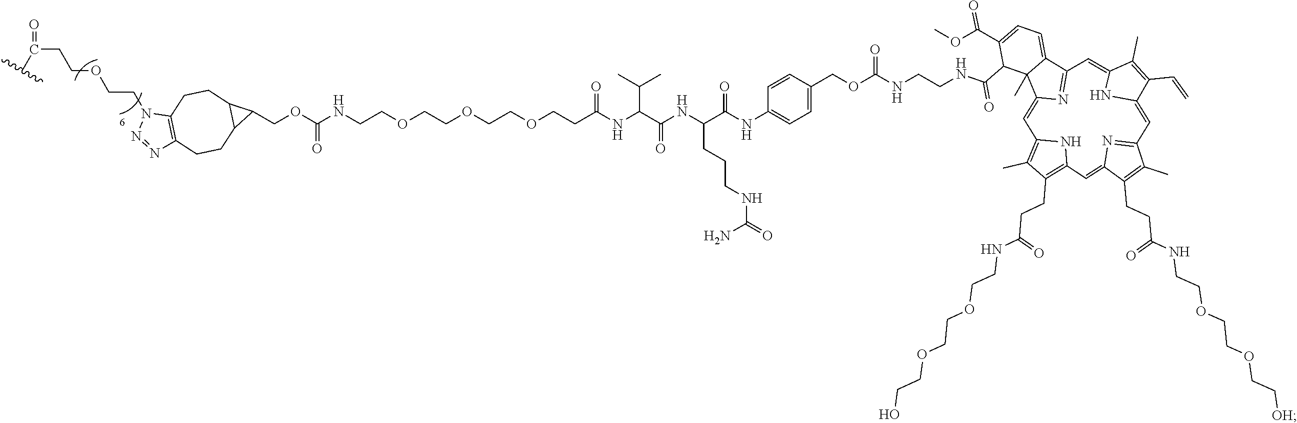

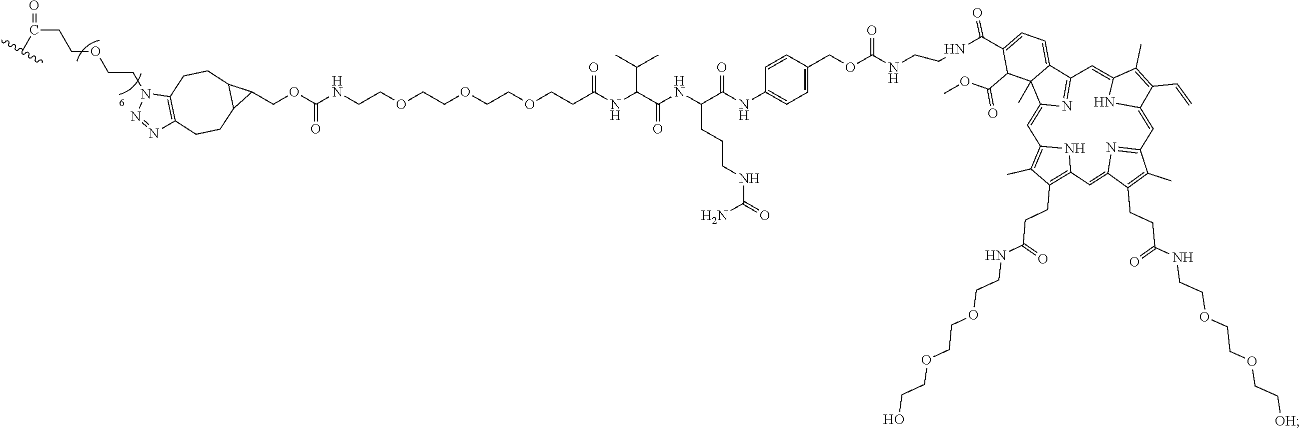

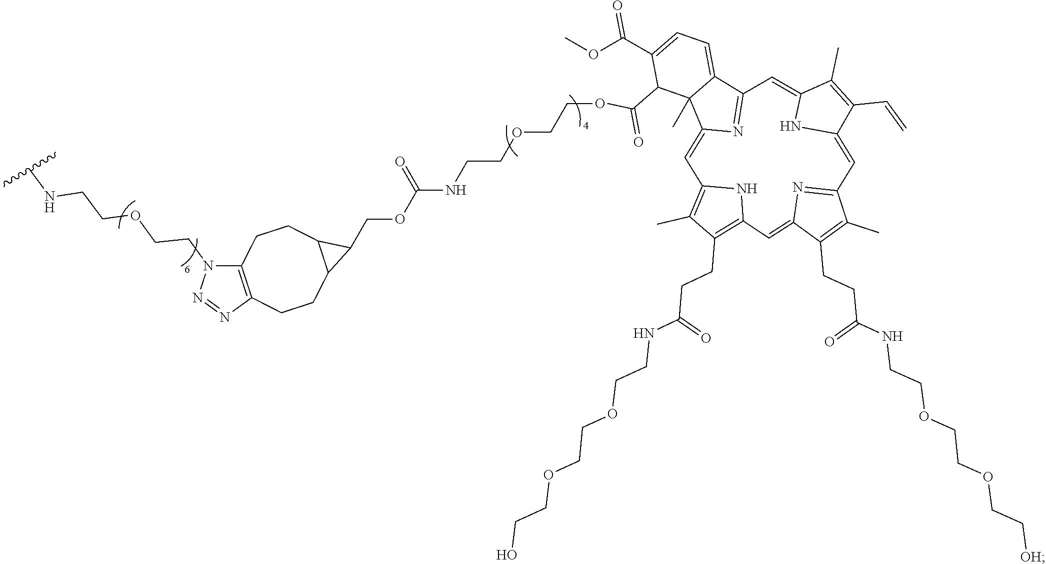

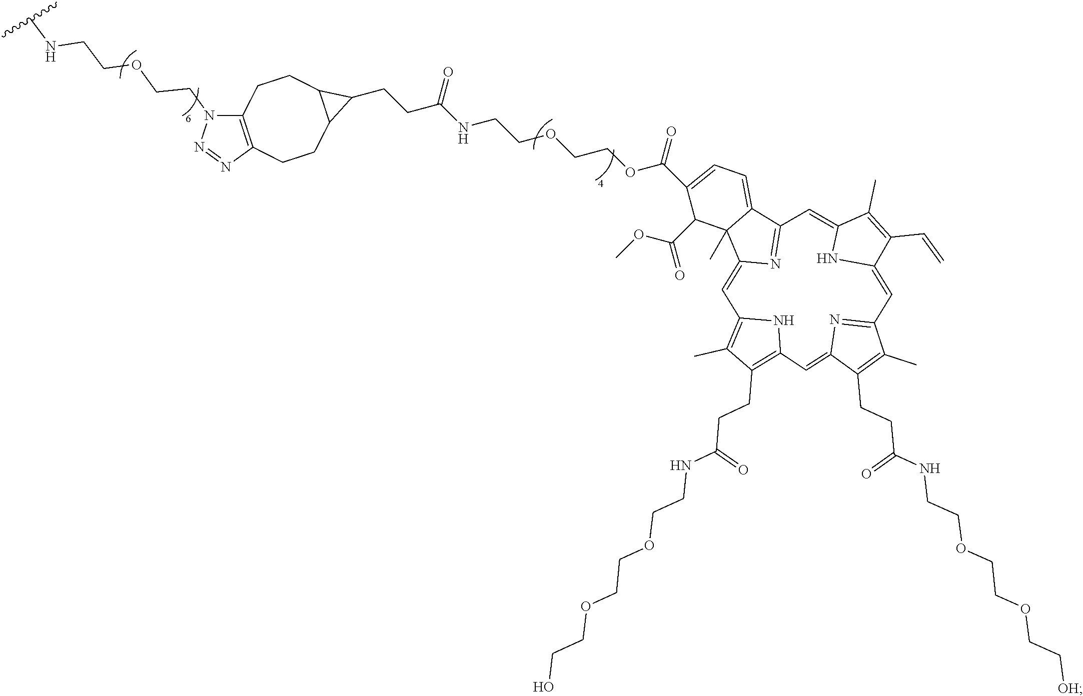

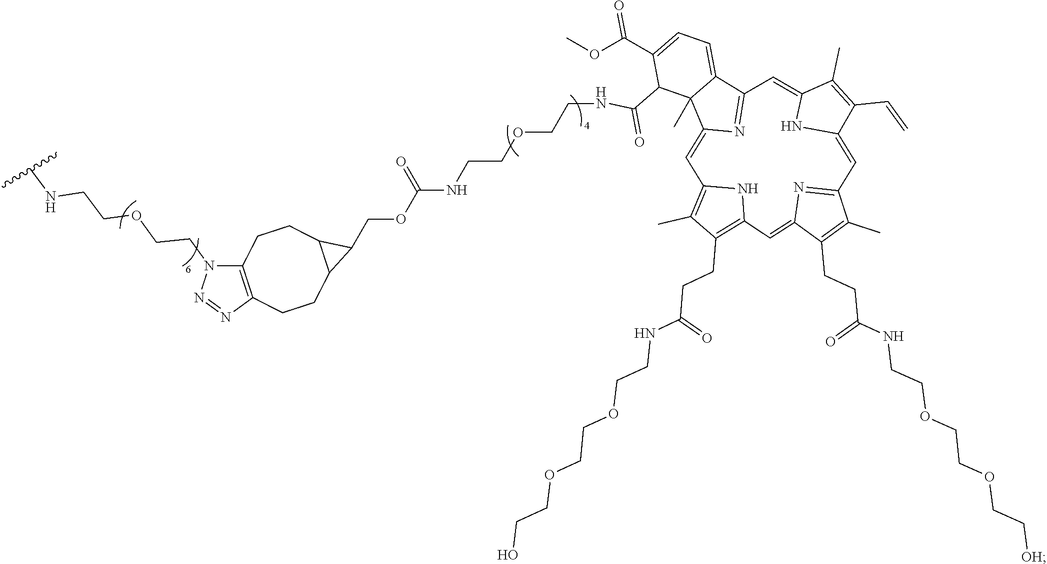

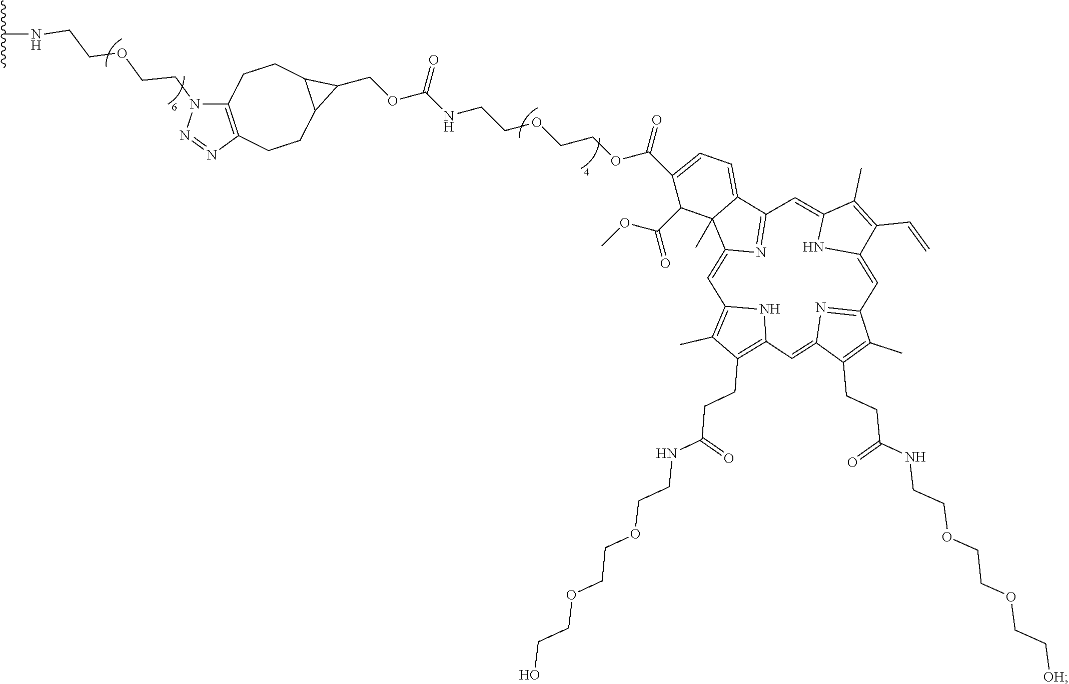

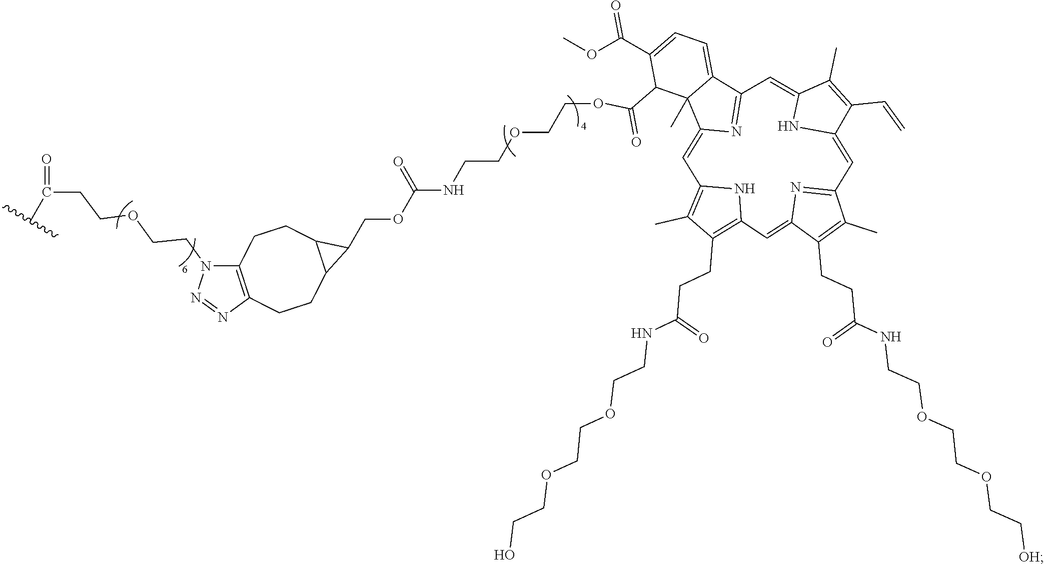

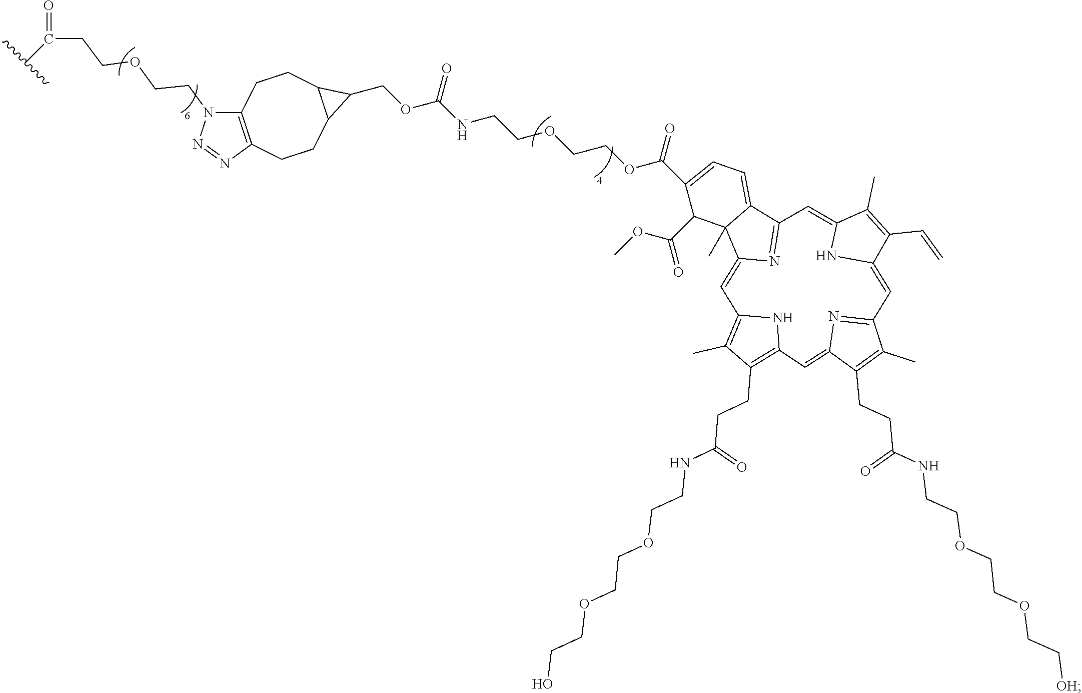

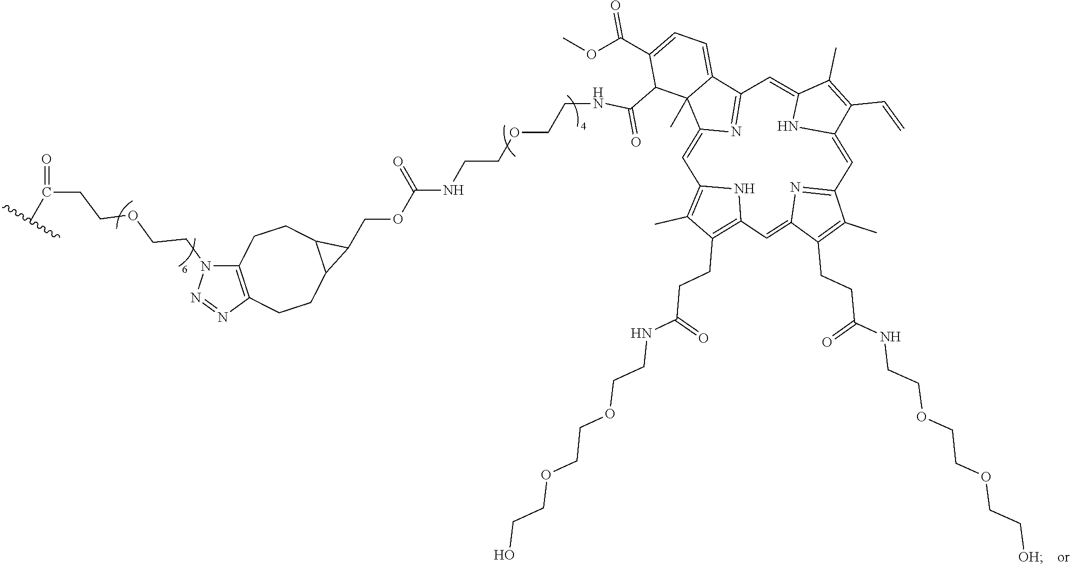

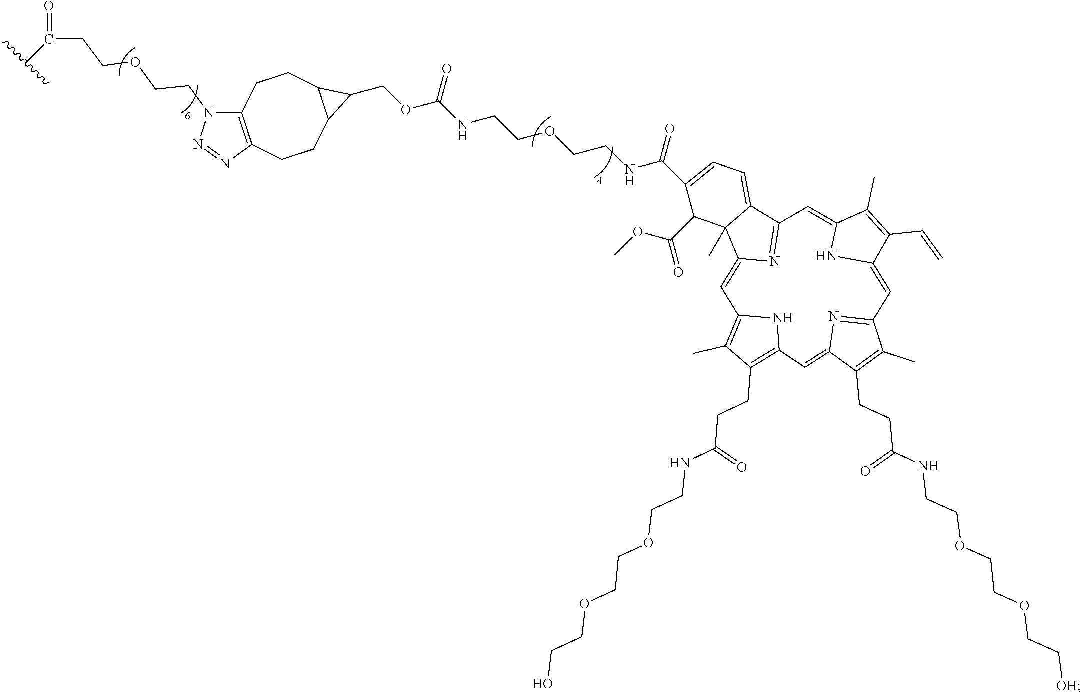

or a pharmaceutically acceptable salt thereof, wherein

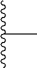

indicates a bond to the targeting agent.

Embodiment 115. The conjugate of embodiment 67, or a pharmaceutically acceptable salt thereof, wherein -L-PS is represented by the following formula:

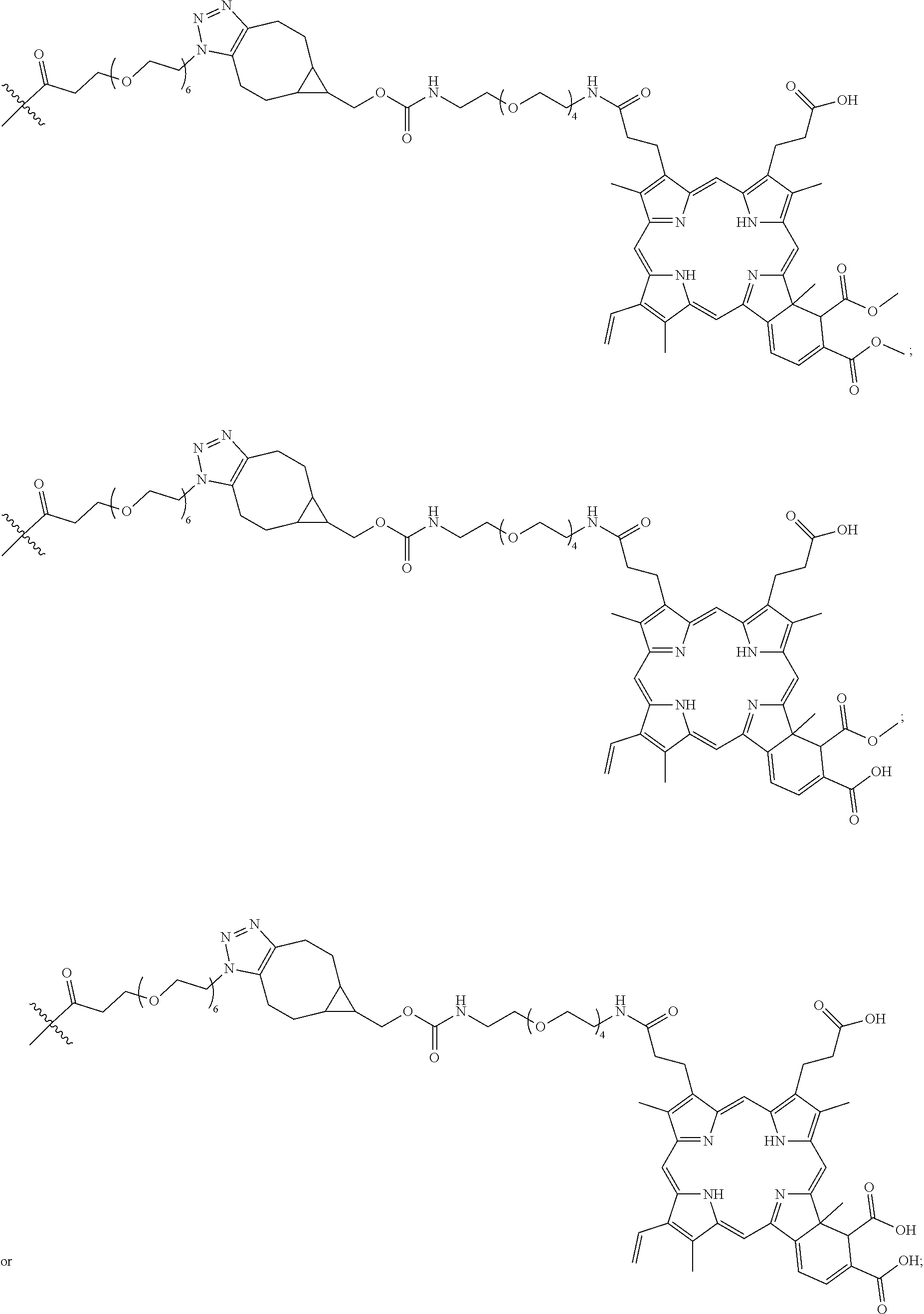

or a pharmaceutically acceptable salt thereof, wherein

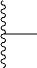

indicates a bond to the targeting agent.

Embodiment 116. A pharmaceutical composition comprising a conjugate of any one of embodiments 69-115, or a pharmaceutically acceptable salt thereof, and a pharmaceutically acceptable excipient.

Embodiment 117. A method of treating a subject having a disease or condition comprising:
a) administering to the subject a therapeutically effective amount of the conjugate of any one of embodiments 69-115, or a pharmaceutically acceptable salt thereof, or a pharmaceutical composition of embodiment 116; and
b) after administering the conjugate or a pharmaceutically acceptable salt thereof or a pharmaceutical comprising the conjugate or a pharmaceutically acceptable salt thereof, illuminating a target region of the subject with a near infrared (NIR) light sufficient to activate the photosensitizer of the conjugate.

Embodiment 118. The method of embodiment 117, wherein the NIR light is at a wavelength between 600 nm to 850 nm.

Embodiment 119. The method of embodiment 117, wherein the NIR light is at a wavelength between 660 nm to 740 nm.

Embodiment 120. The method of any one of embodiments 117-119, wherein the illumination is performed at least 5 minutes after administration of the conjugate.

Embodiment 121. The method of embodiment 116, wherein the illumination is performed between 1 hour and 48 hours, between 4 hours and 30 hours or between 8 hours and 24 hour after administration of the conjugate.

Embodiment 122. The method of any one of embodiments 117-121, wherein the target region is a tumor cell, a mass of tumor cells, a solid tumor, in the vicinity of a solid tumor, a metastasis, a metastasized tumor cell, in the vicinity of a metastasis or a pre-cancerous lesion.

Embodiment 123. The method of any one of embodiments 117-122, wherein the disease or condition is a cancer.

Embodiment 124. The method of embodiment 123, wherein the cancer is selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, esophogeal, gastrointestinal cancer, stomach (gastric) cancer, cancer of the small intestine, colon cancer, spindle cell neoplasm, hepatic carcinoma, liver cancer, cholangiocarcinoma, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma.

Embodiment 125. The method of any one of embodiments 117-121, wherein the disease or condition is an inflammatory or autoimmune condition/disease.

Embodiment 126. The method of any one of embodiments 117-121, wherein the target region is skin lesions.

Embodiment 127. The method of embodiment 126, wherein the skin lesions is skin lesions associated with psoriasis, atopic dermatitis, lupus, vitiligo, graft-versus-host disease, cutaneous T-cell lymphoma, contact dermatitis, cutaneous hypersensitivity response, lichen planus, lichen planopilaris, rejection of vascularized composite allografts, alopecia areata, scarring alopecia or sarcoid.

Embodiment 128. The method of any one of embodiments 117-121, wherein the target region is kidney of the subject.

Embodiment 129. The method of any one of embodiments 117-121 or 128, wherein the disease or condition is T cell mediated kidney or renal pathology/disease associated with lupus nephritis, autoimmune nephritis or kidney graft rejection.

Embodiment 130. The method of any one of embodiments 117-121, wherein the target region is gut or bowel of the subject.

Embodiment 131. The method of any one of embodiments 117-121 or 130, wherein the disease or condition is gastrointestinal inflammation as a result of an autoimmune and/or an inflammatory condition, e.g., inflammatory bowel disease (Crohn's disease, ulcerative colitis).

Embodiment 132. The method of any one of embodiments 117-121, wherein the target region is inflamed joints of the subject.

Embodiment 133. The method of any one of embodiments 117-121 or 132, wherein the disease or condition is joint inflammation from rheumatoid arthritis or spondyloarthritides.

Embodiment 134. A method of imaging a cell or tissue having a target molecule in a subject, the method comprising:
a) administering to a subject the conjugate of any one of embodiments 69-115, or a pharmaceutically acceptable salt thereof, or a pharmaceutical composition of embodiment 116; and
b) after administering the conjugate or a pharmaceutically acceptable salt thereof or a pharmaceutical comprising the conjugate or a pharmaceutically acceptable salt thereof, illuminating a target region of the subject with a near infrared (NIR) light sufficient to activate the photosensitizer of the conjugate, thereby providing an image of the presence of the target molecule associated with the cell or tissue.

Embodiment 135. A compound represented by the following formula:

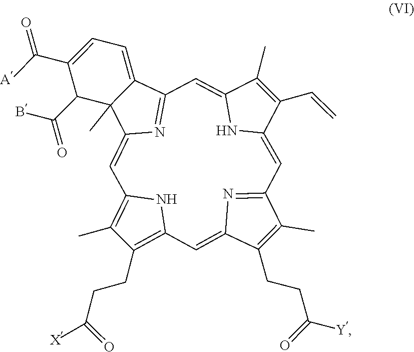

or a pharmaceutically acceptable salt thereof, wherein:
A′, B′, X′ and Y′ are each independently —OH, —OC₁₋₄alkyl, —N(R¹⁰⁰)(R¹⁰¹), or —Z—C₁₋₆allkylene-NH₂, provided one of A′, B′, X′ and Y′ is —Z—C₁₋₆allkylene-NH₂;
R¹⁰⁰ is H or C₁₋₃alkyl;
R¹⁰¹ is H, C₁₋₃alkyl or —(CH₂—CH₂—O)ₙᵣR¹⁰²;
R¹⁰² is H or Me;
nr is an integer from 1 to 16;
Z is —O— or —NR¹—; and
R¹ is H or C₁₋₃alkyl.

Embodiment 136. The compound of embodiment 135, or a pharmaceutically acceptable salt thereof, wherein:
a) A′ and B′ are —OCH₃; and one of X′ and Y′ is —OCH₃ and the other one of X′ and Y′ is —Z—C₁₋₆allkylene-NH₂;
b) A′ and B′ are —OCH₃; and one of X′ and Y′ is —OH and the other one of X′ and Y′ is —Z—C₁₋₆allkylene-NH₂
c) one of A′ and B′ is —OCH₃, and the other one of A′ and B′ is —OH; and one of X′ and Y′ is —OH and the other one of X′ and Y′ is —Z—C₁₋₆allkylene-NH₂;
d) A′ and B′ are —OH; and one of X′ and Y′ is —OH and the other one of X′ and Y′ is —Z—C₁₋₆allkylene-NH₂; or
e) one of A′ and B′ is —OCH₃ and the other one of A′ and B′ is —Z—C₁₋₆allkylene-NH₂; and X′ and Y′ are both —NH(CH₂—CH₂—O)ₙᵣR¹⁰², wherein R¹⁰² is H or Me; and nr is an integer from 2 to 8.

Embodiment 137. The compound of embodiment 135, or a pharmaceutically acceptable salt thereof, wherein the compound is represented by the following formula:

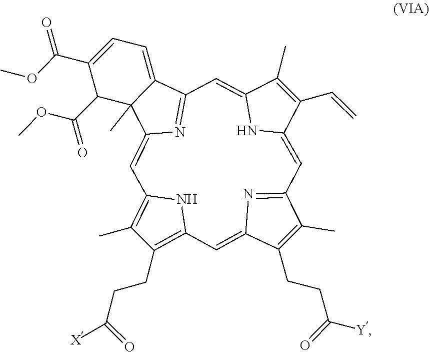

or a pharmaceutically acceptable salt thereof, wherein one of X′ and Y′ is —Z—C₁₋₆allkylene-NH₂ and the other is —OH.

Embodiment 138. The compound of embodiment 135, or a pharmaceutically acceptable salt thereof, wherein the compound is represented by the following formula:

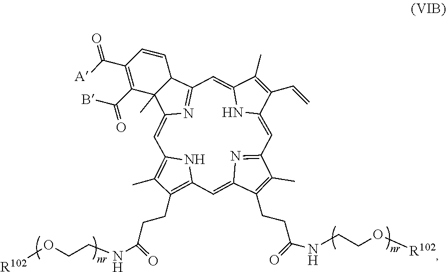

or a pharmaceutically acceptable salt thereof, wherein one of A′ and B′ is —Z—C₁₋₆allkylene-NH₂ and the other is —OH or —OCH₃.

Embodiment 139. The compound of any one of embodiments 135-138, or a pharmaceutically acceptable salt thereof, wherein Z is —NH—.

Embodiment 140. The compound of any one of embodiments 1335-138, or a pharmaceutically acceptable salt thereof, wherein Z is —O—.

Embodiment 141. The compound of embodiment 135, or a pharmaceutically acceptable salt thereof, wherein the compound is:

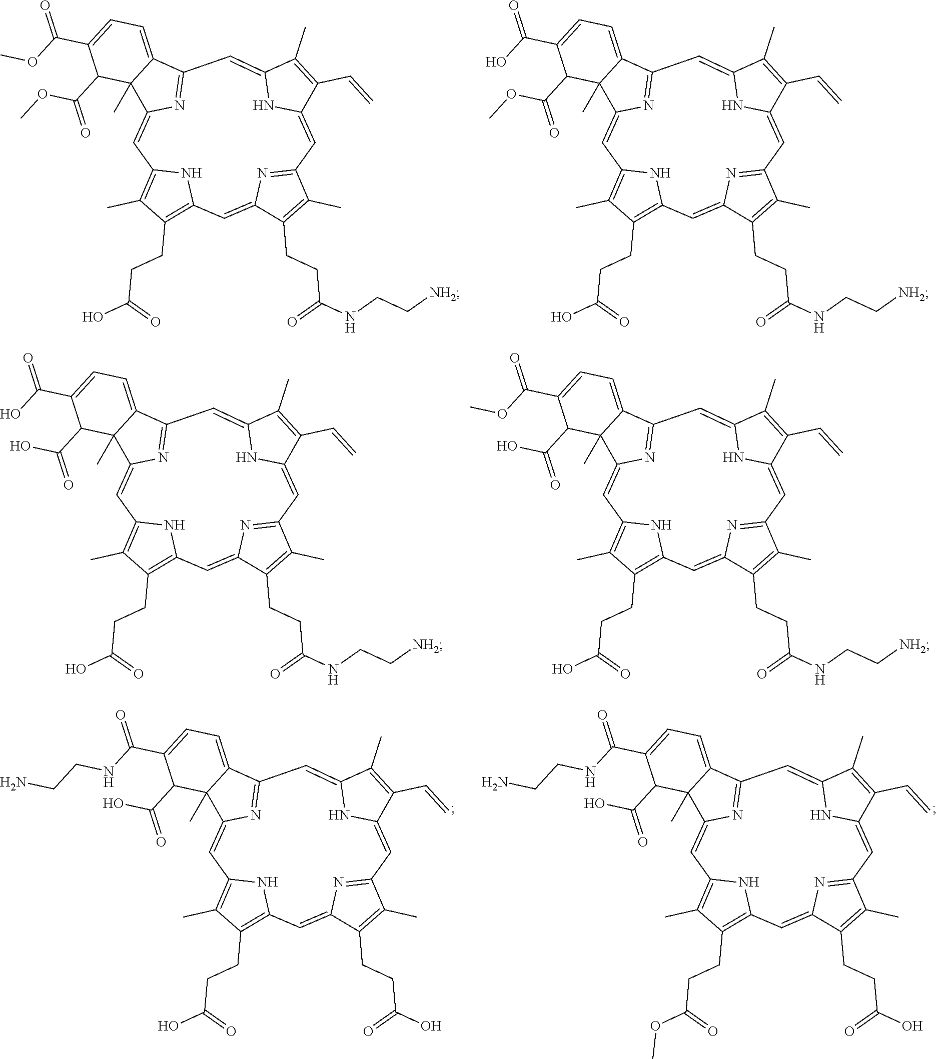

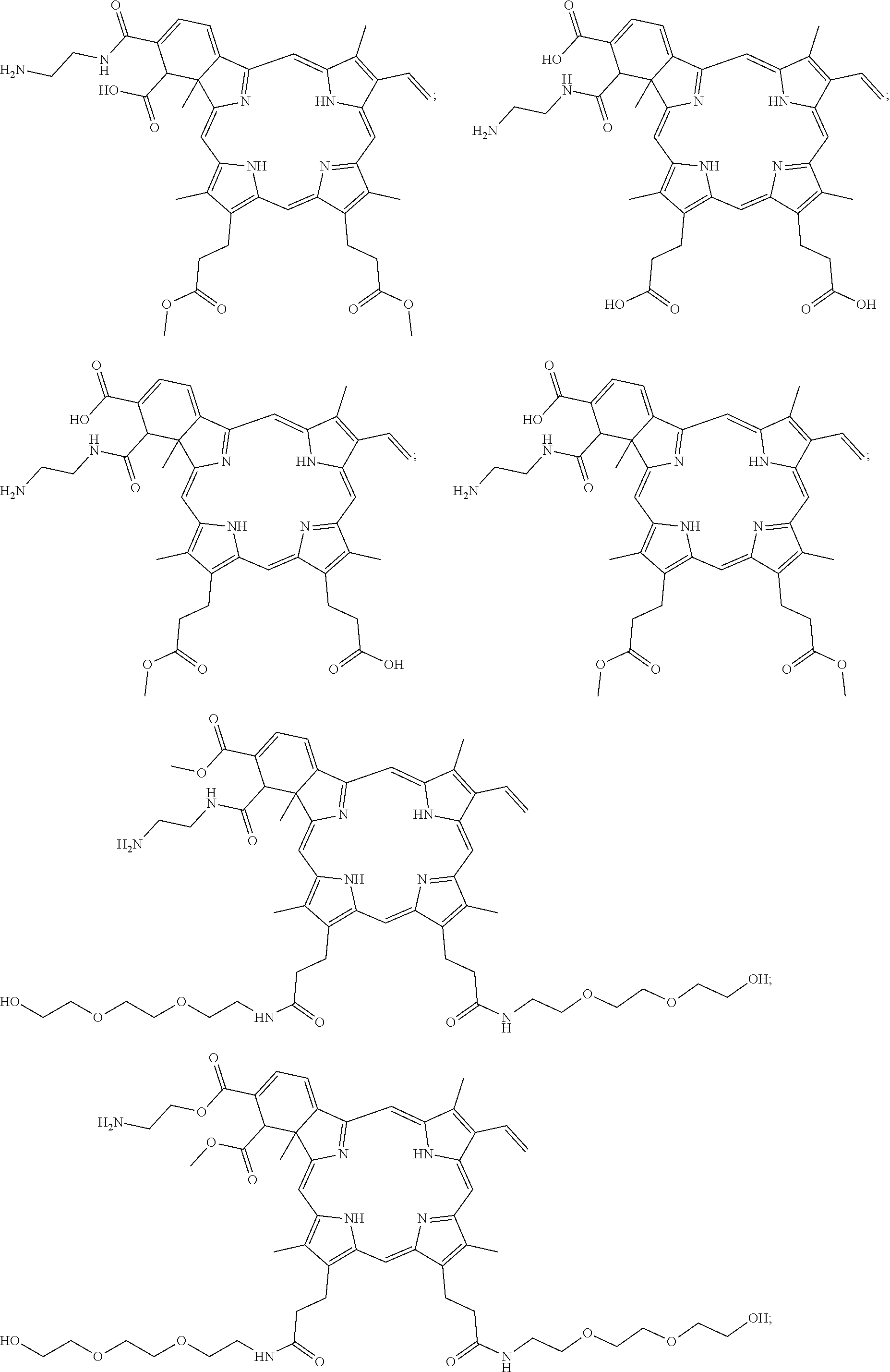

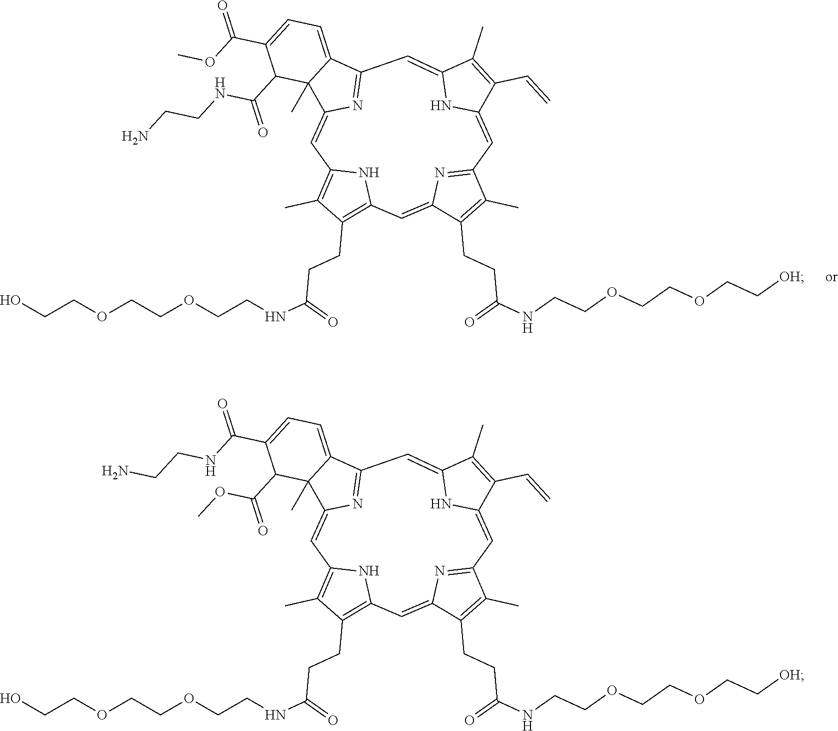 [SMILES: C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)OC)C1C(=O)NCCN ; C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)NCCN)C1C(=O)OC]

or a pharmaceutically acceptable salt thereof, a regioisomer, or a pharmaceutically acceptable salt of the regioisomer.

Embodiment 142. The compound of embodiment 135, or a pharmaceutically acceptable salt thereof, wherein the compound is:

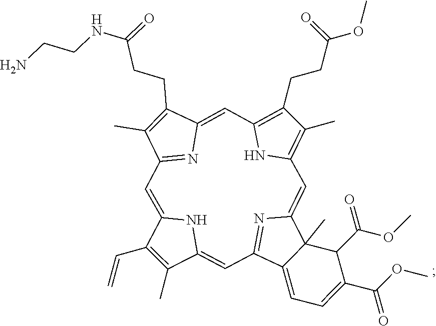 [SMILES: C=Cc1c(C)c2cc3nc(cc4[nH]c(cc5nc(cc1[nH]2)C(C)=C5CCC(=O)NCCN)c(CCC(=O)OC)c4C)C1(C)C3=CC=C(C(=O)OC)C1C(=O)OC]

or a pharmaceutically acceptable salt thereof, a regioisomer, or a pharmaceutically acceptable salt of the regioisomer.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The examples provided do not in any way limit the disclosure.

### Example 1. Synthesis of Novel Benzoporphyrin Analogs

Only a single regioisomer is shown for all the structures below. The specific regioisomers shown below are arbitrarily assigned.

### Example 1a

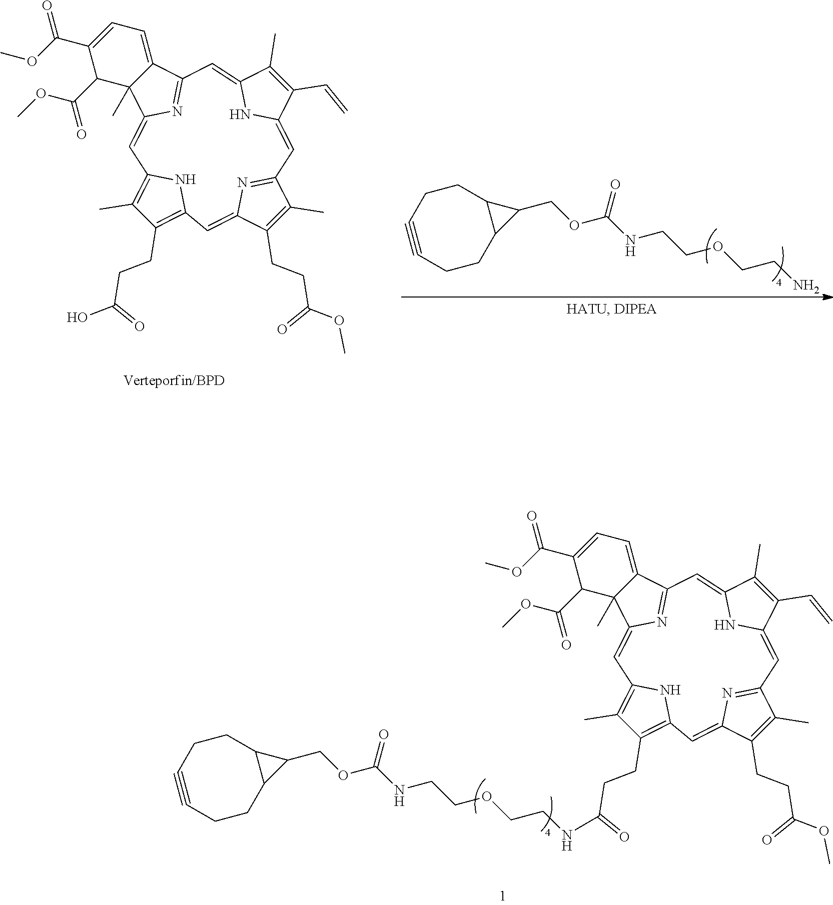

To a solution of verteporfin (230 mg, 0.32 mmol; 1711461, USP) in 10 ml DMF was added HATU (134 mg, 0.35 mmol) and DIPEA (167 ul, 0.96 mmol) at r.t. The reaction mixture was stirred for 30 min and endo-BCN-PEG₄-NH₂ (132 mg, 0.32 mmol; BroadPharm, BP-24459) was added. The mixture was stirred for 2 h and diluted with EtOAc. The mixture was washed with water, brine, dried over Na₂SO₄ and concentrated. The residue was purified by reverse phase chromatography to give the desired compound 1 (PS-0001; 255 mg, 0.23 mmol).

¹H NMR (500 MHz, DMSO-d6): δ 9.88 (d, 1H, J=5), 9.82 (d, 1H, J=10), 9.67 (d, 1H, J=5), 9.31 (d, 1H, J=5), 8.38 (dd, 1H, J=15, J=10), 7.77 (m, 3H), 7.04 (m, 1H), 6.47 (d, 1H, J=20), 6.20 (d, 1H, J=10), 5.25 (s, 1H), 4.40-4.04 (m, 6H), 3.91 (s, 3H), 3.61 (s, 3H), 3.57 (s, 3H), 3.48 (s, 3H), 3.40-3.15 (m, 21H), 3.07 (m, 2H), 3.00-2.85 (m, 5H), 2.66-2.50 (m, 2H), 2.20-2.06 (m, 6H), 1.77 (s, 3H), 1.45 (m, 2H), 1.25 (m, 1H), 0.78 (m, 2H), −2.50 (m, 2H); MS calcd for C₆₂H₇₆N₆O₁₃ ½[M+Na]=567.8, found 568.0.

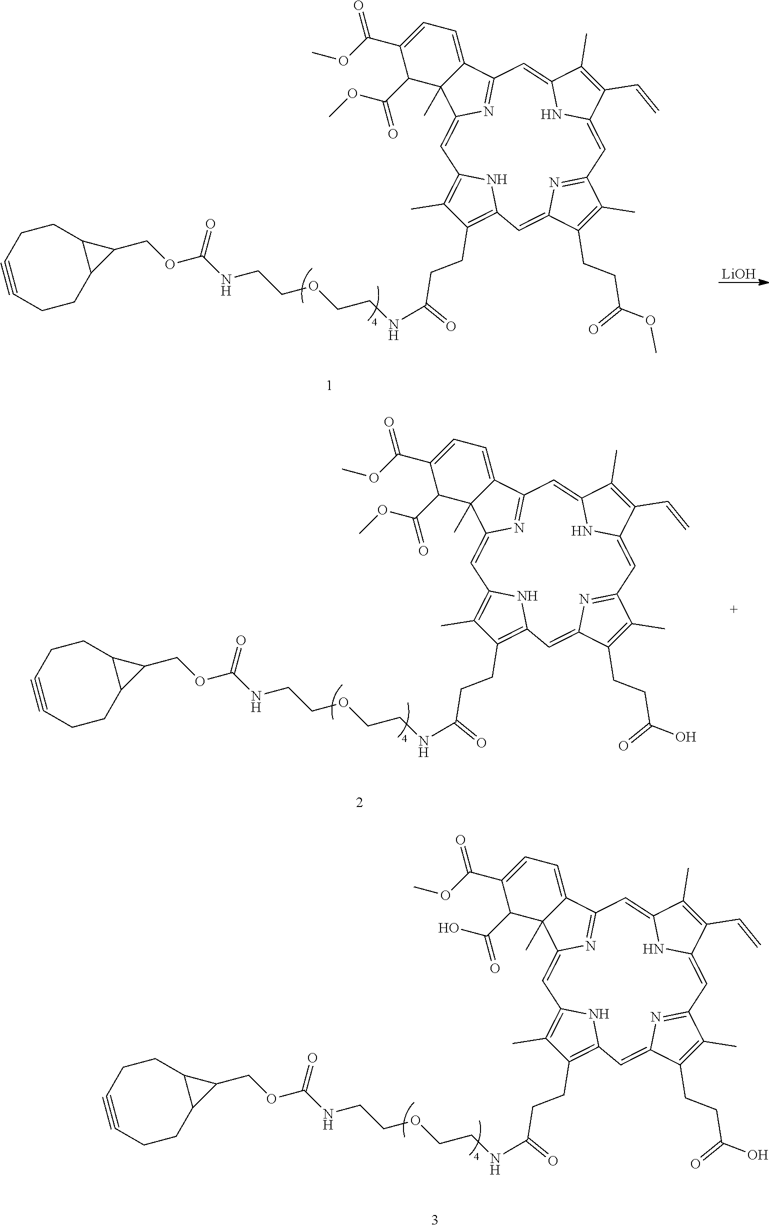

To a solution of compound 1 (36 mg, 0.032 mmol) in 10 mL THF was added LiGH (2N, 2 ml) at 0° C. The reaction mixture was stirred for 6 h at RT. THE was removed and the residue was purified by reverse phase chromatography to give compound 2 (PS-0002; 15 mg, 0.014 mmol) and compound 3 (PS-0003; 8 mg, 0.007 mmol). This purification used Biotage 30 g Cartridge, KP—C₁₈ with MeCN/H₂O as mobile phase running a gradient from 5-95% with 25 mL/min. Compound 3 was eluted out first followed by compound 2.

Compound 2: ¹H NMR (500 MHz, DMSO-d6): δ 10.63 (m, 1H), 10.41 (s, 1H), 10.36 (s, 1H), 9.85 (s, 1H), 9.63 (s, 1H), 9.25 (d, 1H, J=10), 8.38 (dd, 1H, J=15, J=10), 7.76 (s, 1H), 7.14 (m, 1H), 6.45 (d, 1H, J=15), 6.18 (d, 1H, J=10), 5.22 (s, 1H), 4.19-3.94 (m, 6H), 3.90 (s, 3H), 3.61 (s, 3H), 3.47-3.21 (m, 24H), 3.07 (m, 2H), 3.00-2.85 (m, 5H), 2.66-2.50 (m, 2H), 2.20-2.06 (m, 6H), 1.76 (s, 3H), 1.42 (m, 2H), 1.23 (m, 1H), 0.76 (m, 2H), −2.25 (m, 2H); MS calcd for C61H74N6O13 [M+H]=1099.5, found 1099.4.

Compound 3: ¹H NMR (500 MHz, DMSO-d6): δ 10.28 (s, 1H), 10.26 (s, 1H), 9.77 (s, 1H), 9.48 (s, 1H), 9.04 (d, 1H, J=10), 8.35 (dd, 1H, J=15, J=10), 7.61 (d, 1H, J=5), 7.24 (d, 1H, J=5), 7.12 (m, 1H), 6.44 (d, 1H, J=15), 6.17 (d, 1H, J=10), 5.09 (s, 1H), 4.22 (m, 2H), 3.96 (m, 4H), 3.58 (s, 3H), 3.43-3.21 (m, 24H), 3.17 (m, 1H), 3.08 (m, 2H), 2.95 (m, 1H), 2.86 (s, 3H), 2.63 (m, 2H), 2.27-2.06 (m, 6H), 1.67 (s, 3H), 1.45 (m, 2H), 1.17 (m, 1H), 0.76 (m, 2H), −2.25 (m, 2H); MS calcd for C60H72N6O13 [M+H]=1085.5, found 1085.4.

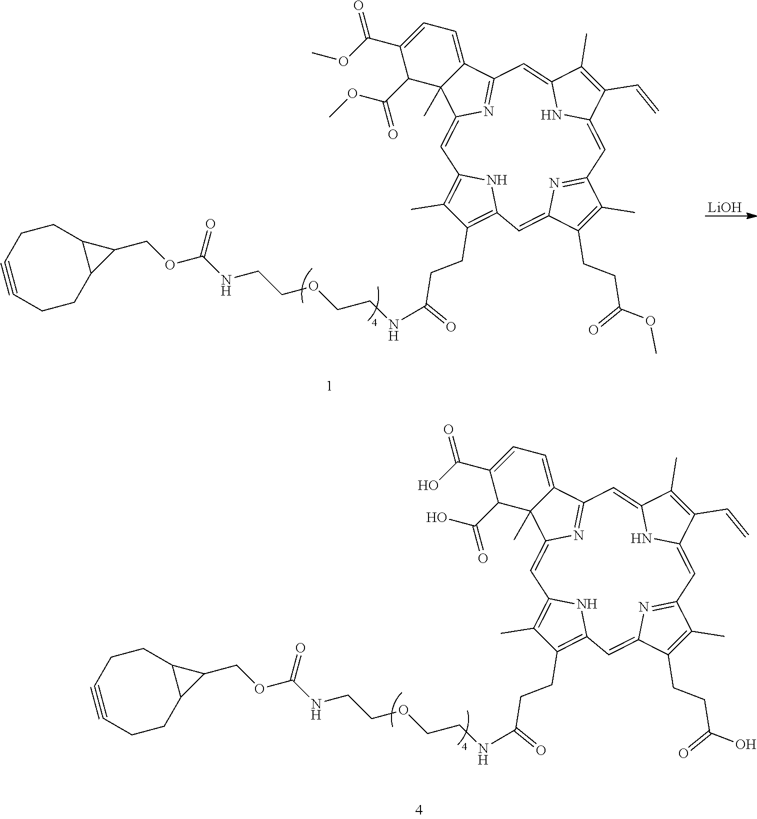

To a solution of compound 1 (33 mg, 0.03 mmol) in 10 mL THE was added LiOH (2N, 2 ml) at 0° C. The reaction mixture was stirred for 10 days at r.t. THE was removed and the residue was purified by reverse phase chromatography to give compound 4 (PS-0004; 18 mg, 0.017 mmol). ¹H NMR (500 MHz, DMSO-d6): δ 10.11 (s, 1H), 10.04 (s, 1H), 9.68 (s, 1H), 9.48 (s, 1H), 8.80 (d, 1H, J=10), 8.35 (dd, 1H, J=15, J=10), 7.32 (d, 1H, J=5), 7.12 (d, 1H, J=5), 7.12 (m, 1H), 6.44 (d, 1H, J=15), 6.15 (d, 1H, J=10), 5.09 (s, 1H), 4.15-3.96 (m, 6H), 3.43-3.21 (m, 24H), 3.17 (m, 1H), 3.08 (m, 2H), 2.95 (m, 1H), 2.86 (s, 3H), 2.63 (m, 2H), 2.27-2.06 (m, 6H), 1.67 (s, 3H), 1.45 (m, 2H), 1.18 (m, 1H), 0.76 (m, 2H), −2.20 (m, 2H); MS calcd for C59H70N6O13 [M+H]=1071.5, found 1071.4.

### Example 1b

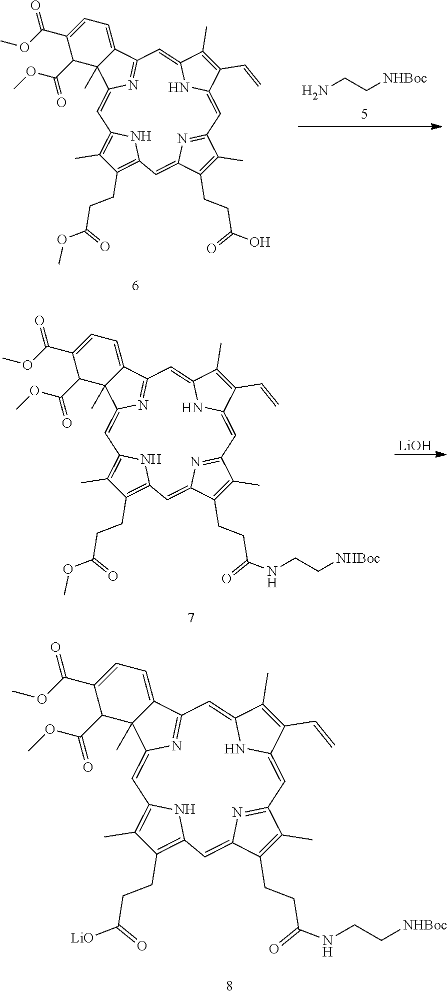

To a solution of verteporfin (compound 6; 270 mg, 0.376 mmol) in 10 ml DMF was added HATU (170 mg, 0.45 mmol) and DIPEA (200 ul, 1.15 mmol) at RT. The reaction mixture was stirred for 30 min and amine (compound 5; 72 mg, 0.45 mmol) was added. The mixture was stirred for 2 h and diluted with EtOAc. The mixture was washed with water, brine, dried over Na₂SO₄ and concentrated. The residue was purified by reverse phase chromatography to give the desired (compound 7; 250 mg, 0.29 mmol). MS calcd for C48H56N6O9, [M+H]=861.4, found 861.4.

To a solution of compound 7 (25 mg, 0.03 mmol) in 3 mL THF was added LiGH (2N, 200 ul) at 0° C. The reaction mixture was stirred for 2 h at RT. THE was removed and the residue was purified by reverse phase chromatography to give the compound 8 (12 mg, 0.014 mmol). MS calcd for C47H54N6O9, [M+H]=847.4, found 847.3.

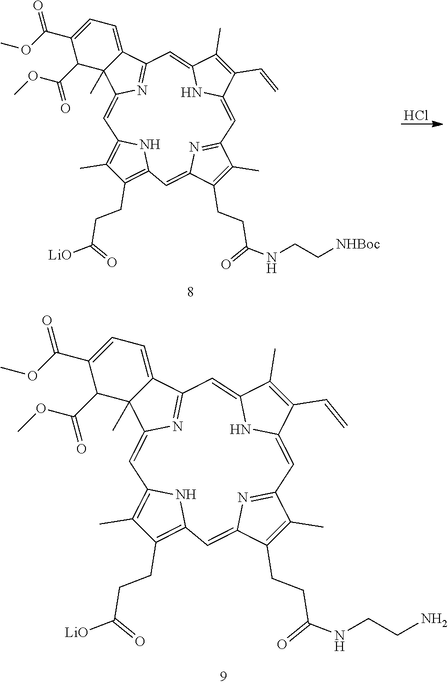

To a solution of compound 8 (12 mg, 0.014 mmol) in 3 mL DCM was added HCl (4N in Dioxane, 1 ml) at 0° C. The reaction mixture was stirred for 1 h at RT. The reaction mixture was concentrated to give the compound 9 (PS-0013; 12 mg, 0.014 mmol). MS calcd for C42H46N6O7, [M+H]=747.3, found 747.3.

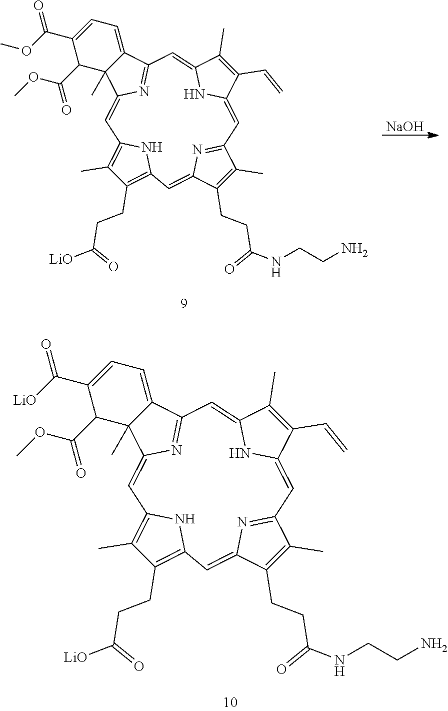

To a solution of compound 9 (25 mg, 0.03 mmol) in 3 mL THF was added LiGH (2N, 400 ul) at 0° C. The reaction mixture was stirred for overnight at RT. THE was removed and the residue was purified by reverse phase chromatography to give compound 10 (20 mg, 0.024 mmol). MS calcd for C46H52N6O9, [M+H]=833.4, found 833.3.

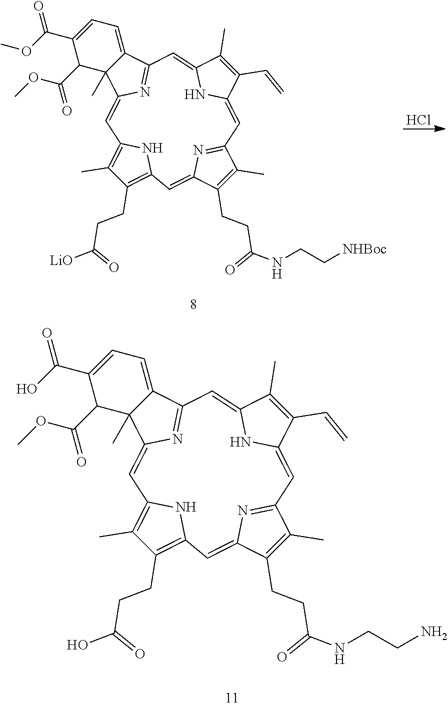

To a solution of compound 8 (20 mg, 0.024 mmol) in 3 mL THE was added HCl (4N in Dioxane, 2 ml) at 0° C. The reaction mixture was stirred for 1 h at RT. The reaction mixture was concentrated to give the compound 11 (PS-0014; 20 mg, 0.024 mmol). MS calcd for C42H46N6O7, [M+H]=733.3, found 733.3.

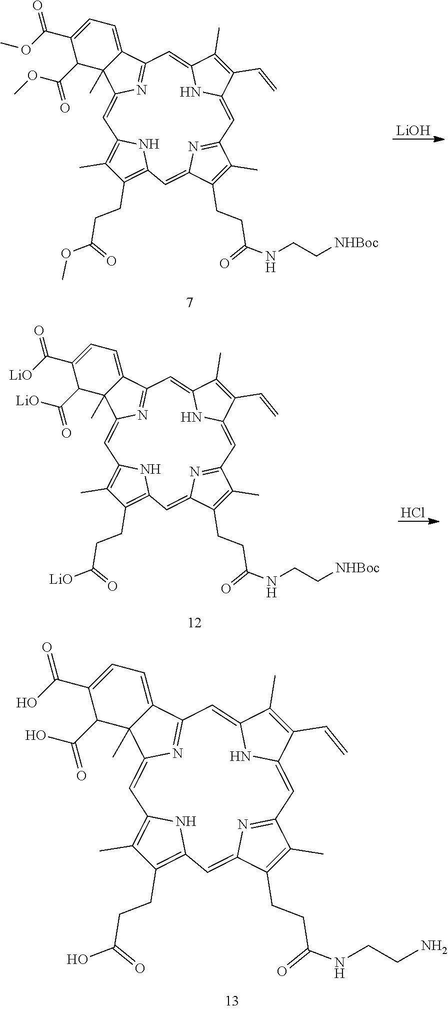

To a solution of compound 7 (25 mg, 0.03 mmol) in 3 mL THF was added LiGH (1N, 800 ul) at 0° C. The reaction mixture was stirred for 10 days at RT. THF was removed and the residue was purified by reverse phase chromatography to give the compound 12 (10 mg, 0.012 mmol). MS calcd for C45H50N6O9, [M+H]=819.4, found 819.4.

To compound 12 (10 mg, 0.012 mmol) was added HCl (4N in Dioxane, 2 ml) at 0° C. The reaction mixture was stirred for 2 hr at RT. The reaction mixture was concentrated to give the compound 13 (PS-0015; 10 mg, 0.012 mmol). MS calcd for C42H46N6O7, [M+H]=719.3, found 719.4.

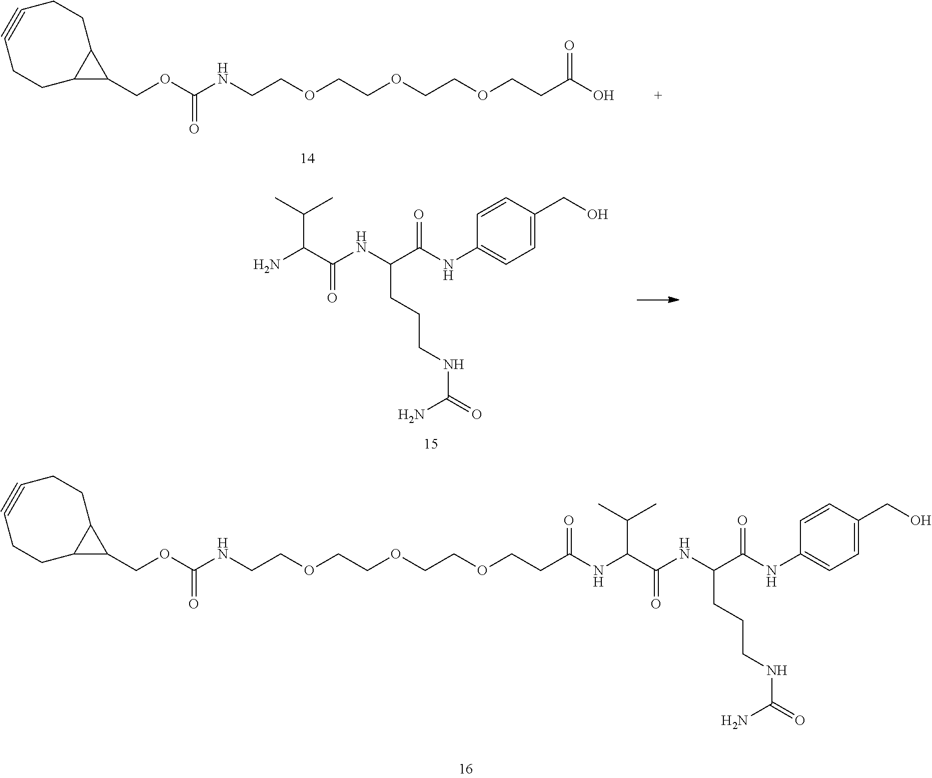 [SMILES: O=C(O)CCOCCOCCOCCNC(=O)OCC1C2CCC#CCCC21 ; CC(C)C(N)C(=O)NC(CCCNC(N)=O)C(=O)Nc1ccc(CO)cc1 ; CC(C)C(NC(=O)CCOCCOCCOCCNC(=O)OCC1C2CCC#CCCC21)C(=O)NC(CCCNC(N)=O)C(=O)Nc1ccc(CO)cc1]

To a solution of Endo-BCN-PEG3-acid (compound 14; 132 mg, 0.332 mmol) in 5 ml DMF was added HATU (130 mg, 0.34 mmol) and DIPEA (173 ul, 1.0 mmol) at RT. The reaction mixture was stirred for 10 min and Val-Cit-PAB-OH (compound 15; 126 mg, 0.332 mmol) was added. The mixture was stirred for 2 h. The mixture was purified by reverse phase chromatography to give compound 16 (170 mg, 0.22 mmol). MS calcd for C38H58N6O10, [M+H]=759.4, found 759.4.

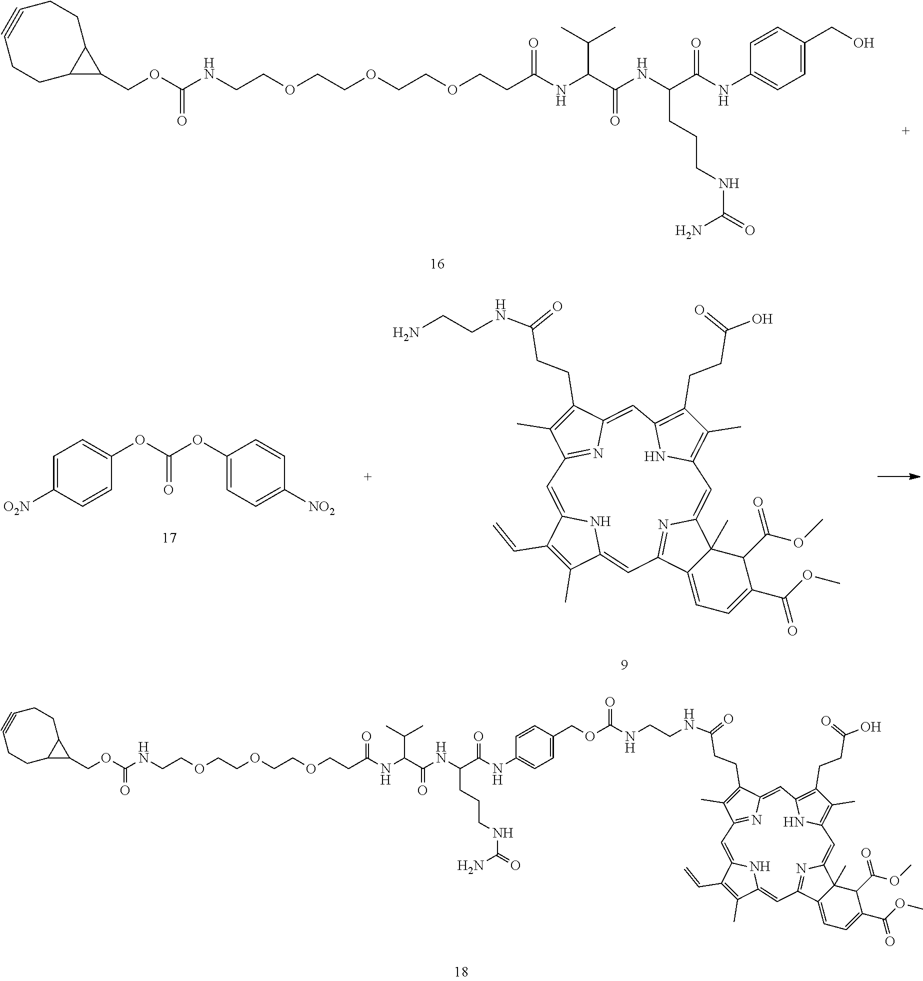 [SMILES: CC(C)C(NC(=O)CCOCCOCCOCCNC(=O)OCC1C2CCC#CCCC21)C(=O)NC(CCCNC(N)=O)C(=O)Nc1ccc(CO)cc1 ; O=C(Oc1ccc([N+](=O)[O-])cc1)Oc1ccc([N+](=O)[O-])cc1 ; C=Cc1c(C)c2cc3nc(cc4[nH]c(cc5nc(cc1[nH]2)C(C)=C5CCC(=O)NCCN)c(CCC(=O)O)c4C)C1(C)C3=CC=C(C(=O)OC)C1C(=O)OC ; C=Cc1c(C)c2cc3nc(cc4[nH]c(cc5nc(cc1[nH]2)C(C)=C5CCC(=O)NCCNC(=O)OCc1ccc(NC(=O)C(CCCNC(N)=O)NC(=O)C(NC(=O)CCOCCOCCOCCNC(=O)OCC2C5CCC#CCCC52)C(C)C)cc1)c(CCC(=O)O)c4C)C1(C)C3=CC=C(C(=O)OC)C1C(=O)OC]

To a solution of compound 16 (78 mg, 0.10 mmol) in 5 mL DMF was added compound 17 (35 mg, 0.11 mmol) and DIPEA (300 ul) at RT. The reaction mixture was stirred for overnight. The reaction mixture was added slowly to HCl salt (compound 9; 90 mg) with DIPEA (300 ul) in DCM (20 ml). After stirring for 1 h, DCM was removed under reduced pressure and purified by reverse phase chromatography and lyophilized to give the final compound 18 (PS-0011; 120 mg, 0.078 mmol). MS calcd for C81H102N12O18, [½M+H]=766.4, found 766.4. 1H NMR (500 MHz, DMSO-d6): δ 9.97 (s, 2H), 9.88 (s, 1H), 9.66 (s, 1H), 9.30 (s, 1H), 8.38 (dd, 1H, J=15, J=15), 8.11 (m, 1H), 7.86 (d, 1H, J=10), 7.77 (m, 2H), 7.56 (d, 2H, J=10), 7.21 (m, 2H), 7.12 (m, 1H), 6.48 (m, 1H), 6.21 (d, 1H, J=15), 5.96 (m, 1H), 5.38 (s, 2H), 5.24 (s, 1H), 4.85 (d, 1H, J=10), 4.35 (m, 1H), 4.21 (m, 3H), 4.01 (m, 4H), 3.90 (s, 3H), 3.61-3.30 (m, 24H), 3.05-2.80 (m, 15H), 2.44-2.37 (m, 2H), 2.19-2.09 (m, 4H), 1.96-1.90 (m, 1H), 1.77 (s, 3H), 1.67 (m, 1H), 1.60-1.30 (m, 3H), 1.22 (m, 2H), 0.98-0.96 (m, 4H), 0.84-0.80 (m, 8H), −2.51 (m, 2H).

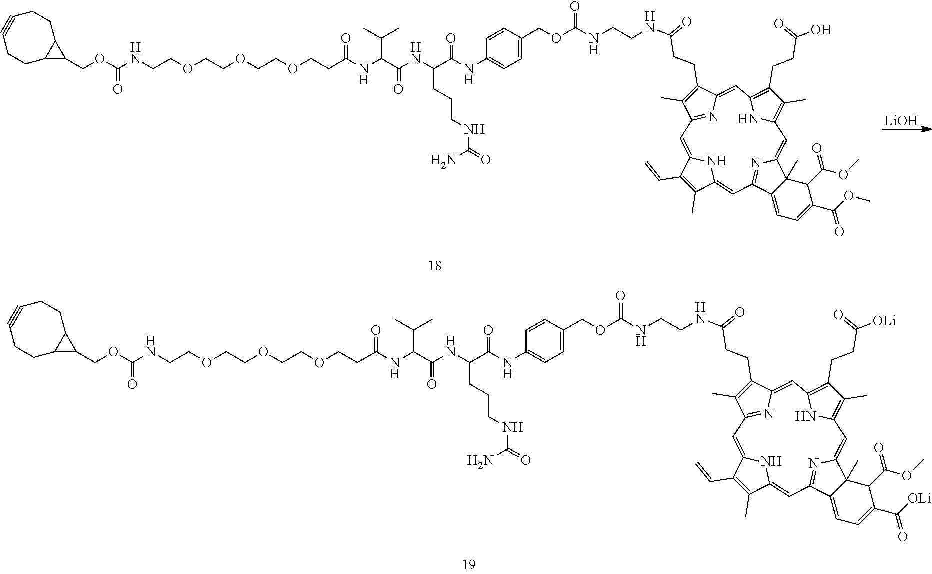

To a solution of compound 18 (10 mg, 0.0065 mmol) in 3 mL THE was added LiGH (2N, 1 ml) at 0° C. The reaction mixture was stirred for 4 h at RT. THE was removed and the residue was purified by reverse phase chromatography to give the compound 19 (PS-0012) (8 mg, 0.005 mmol). MS calcd for C₈₀H100N12O18, [½M+H]=759.4, found 759.4.

### Example 1c

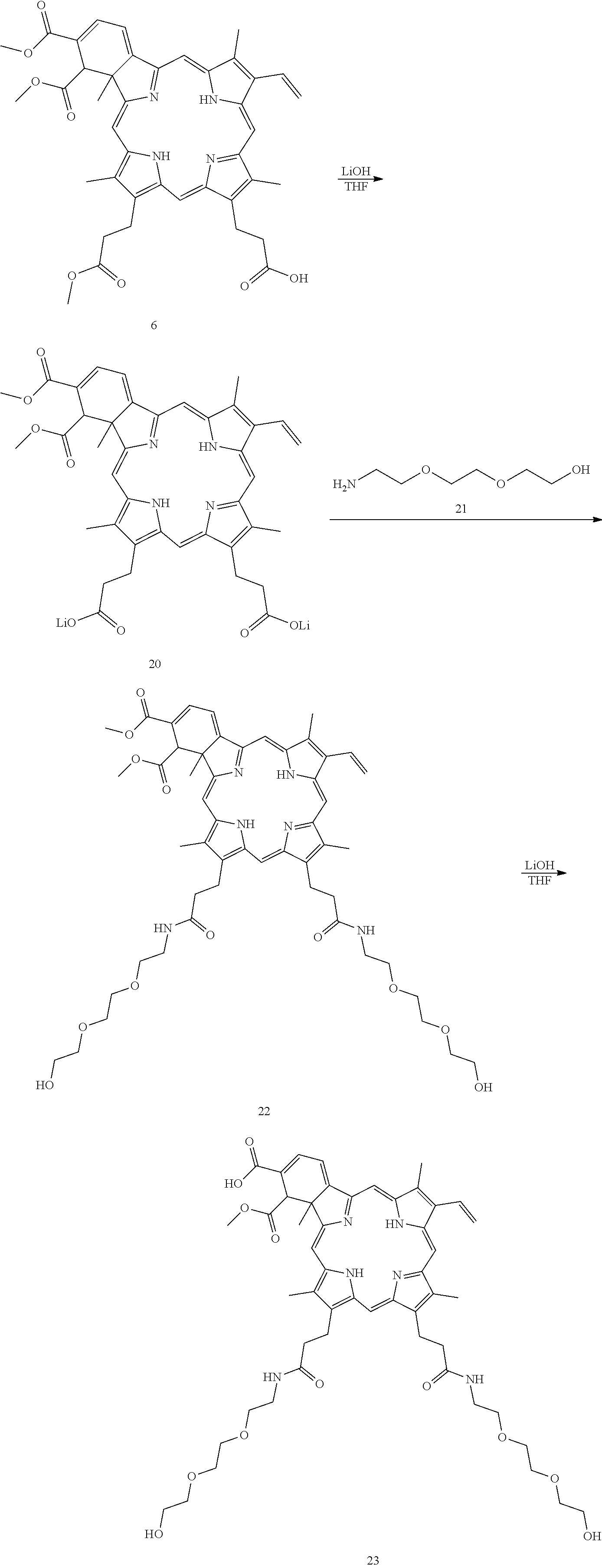

To a solution of verteporfin (compound 6, 72 mg, 0.1 mmol) in 5 mL THF was added LiGH (2N, 2.0 ml) at RT. The reaction mixture was stirred for 1 h at RT. The mixture was purified by reverse phase chromatography to give the compound 20 (60 mg, 0.085 mmol). MS calcd for C40H40N4O8, [M+H]=705.3, found 705.4.

To a solution of compound 20 (60 mg, 0.085 mmol) in 8 ml DMF was added HATU (81 mg, 0.21 mmol) and DIPEA (74 ul, 0.42 mmol) at RT. The reaction mixture was stirred for 30 min and PEG3-amine (compound 21 (32 mg, 0.21 mmol) was added. The mixture was stirred for 2 h. The reaction mixture was purified by reverse phase chromatography to give compound 22 (60 mg, 0.06 mmol). MS calcd for C52H66N6O12, [M+H]=967.5, found 967.4.

To a solution of compound 22 (45 mg, 0.047 mmol) in 2 mL THF was added LiGH (2N, 2.0 ml) at RT. The reaction mixture was stirred for 2 h at RT. The mixture was purified by reverse phase chromatography to give the compound 23 (PS-0016) (28 mg, 0.029 mmol). MS calcd for C51H64N6O12, [M+H]=953.3, found 953.4.

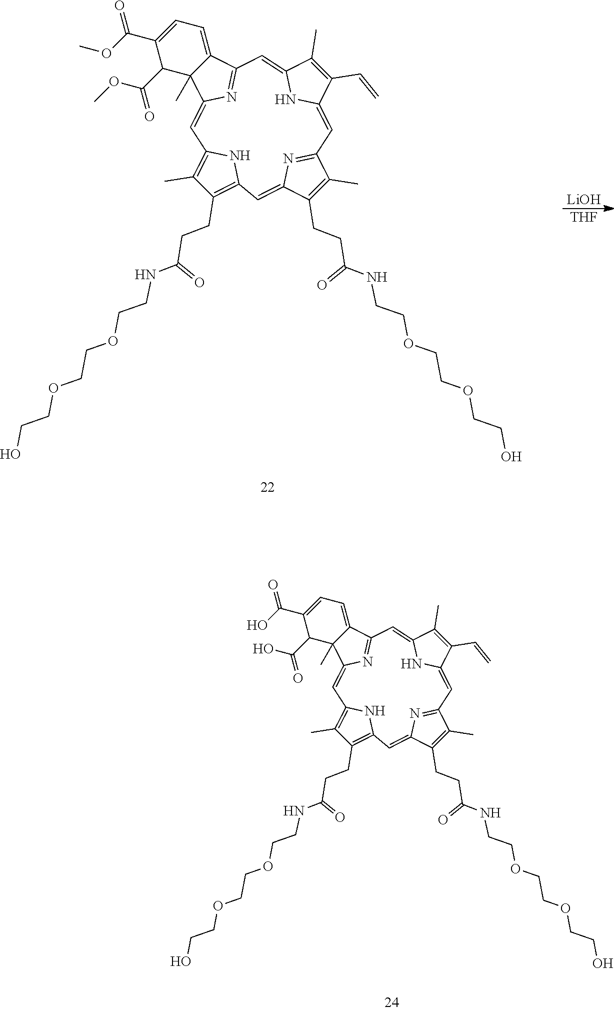 [SMILES: C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)OC)C1C(=O)OC ; "LiOH" ; "THF" ; C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)O)C1C(=O)O]

To a solution of compound 22 (10 mg, 0.01 mmol) in 2 mL THE was added UGH (2N, 2.0 ml) at RT. The reaction mixture was stirred for 4 days at RT. The mixture was purified by reverse phase chromatography to give the compound 24 (PS-0017; 8 mg, 0.0084 mmol). MS calcd for C50H62N6O12, [M+H]=939.4, found 939.4.

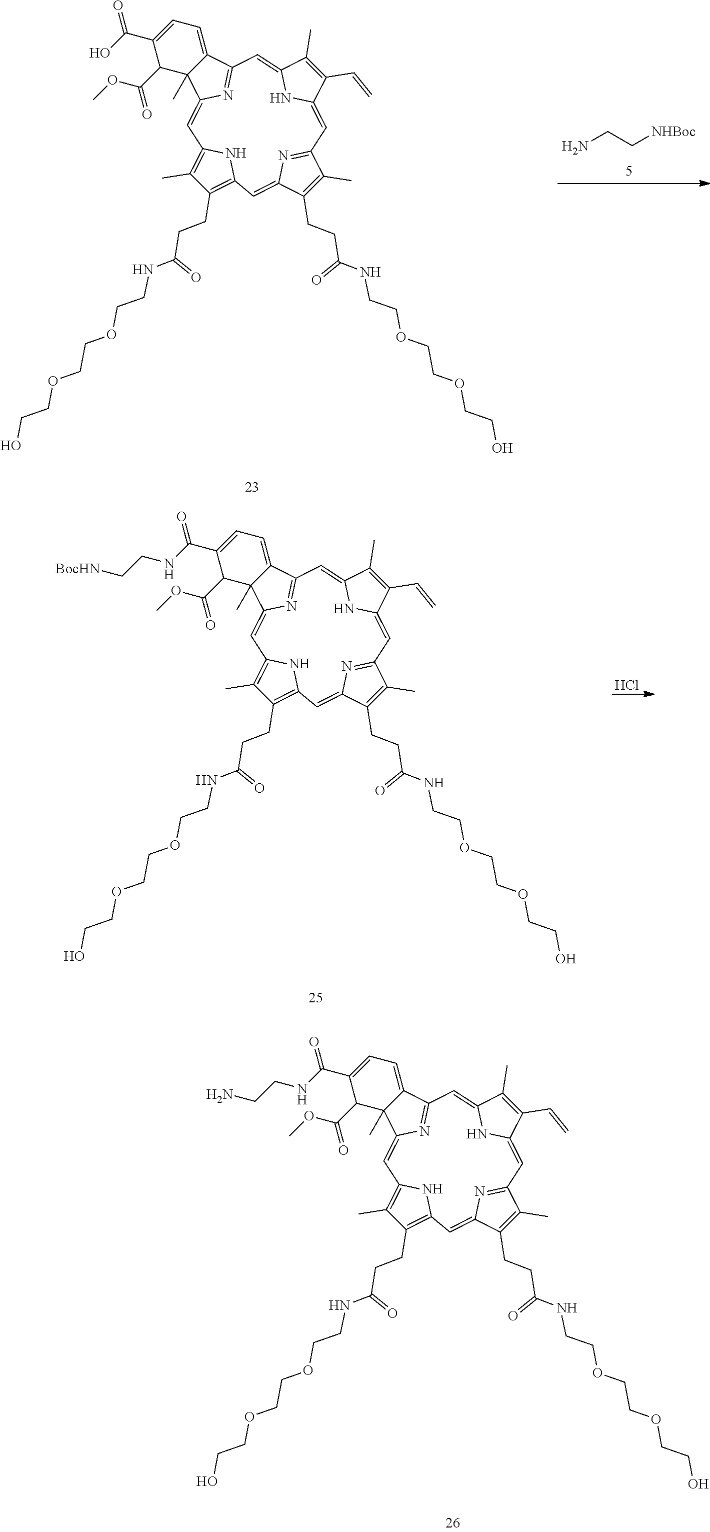 [SMILES: C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)O)C1C(=O)OC ; CC(C)(C)OC(=O)NCCN ; C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)NCCNC(=O)OC(C)(C)C)C1C(=O)OC ; Cl ; C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)NCCN)C1C(=O)OC]

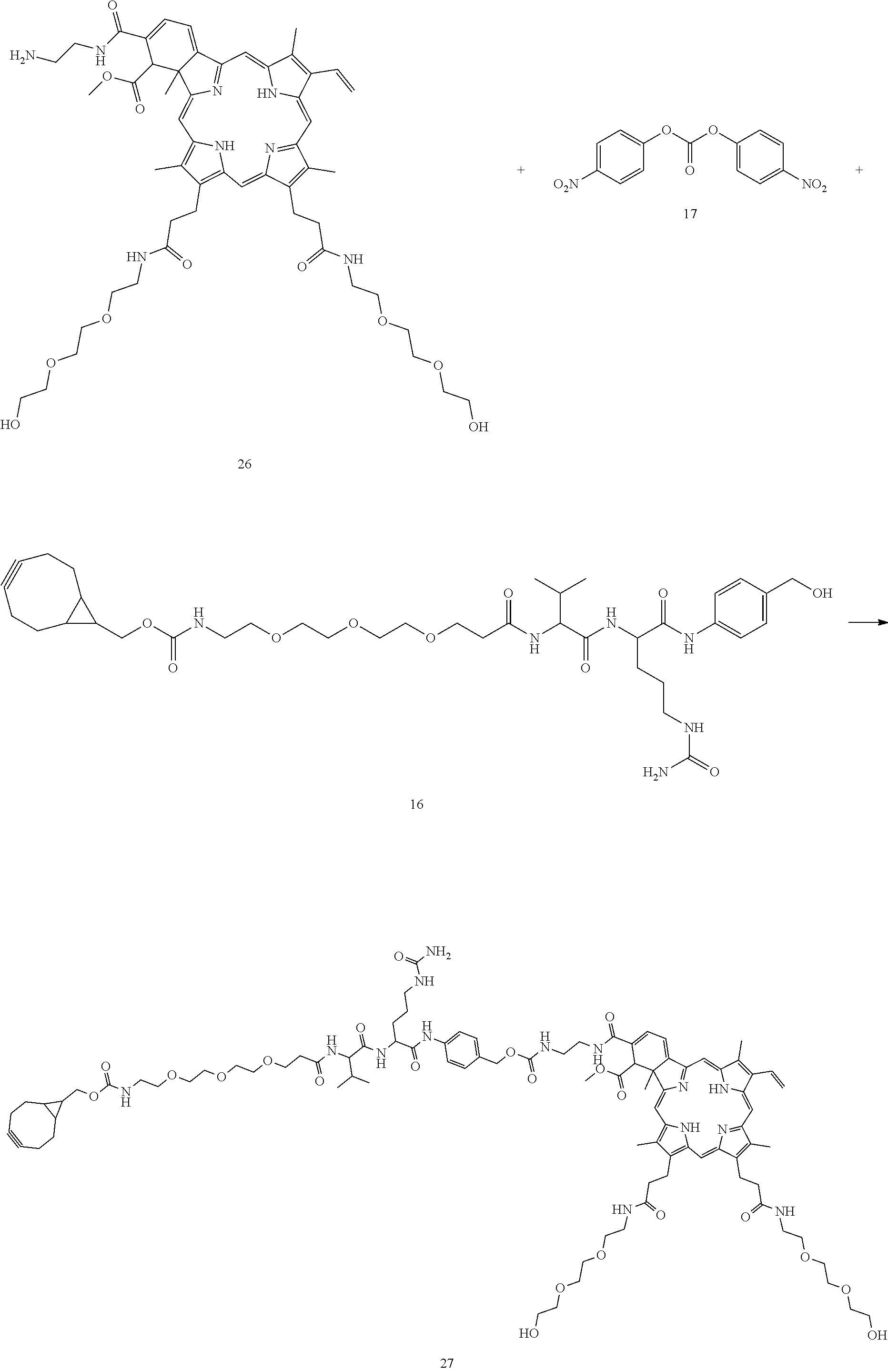 [SMILES: C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)NCCN)C1C(=O)OC ; O=C(Oc1ccc([N+](=O)[O-])cc1)Oc1ccc([N+](=O)[O-])cc1 ; CC(C)C(NC(=O)CCOCCOCCOCCNC(=O)OCC1C2CCC#CCCC21)C(=O)NC(CCCNC(N)=O)C(=O)Nc1ccc(CO)cc1 ; C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)NCCNC(=O)OCc2ccc(NC(=O)C(CCCNC(N)=O)NC(=O)C(NC(=O)CCOCCOCCOCCNC(=O)OCC3C4CCC#CCCC43)C(C)C)cc2)C1C(=O)OC]

To a solution of compound 23 (21 mg, 0.022 mmol) in 4 ml DMF was added HATU (10 mg, 0.026 mmol) and DIPEA (8 ul, 0.046 mmol) at RT. The reaction mixture was stirred for 30 min and amine (compound 5; 4.2 mg, 0.026 mmol) was added. The mixture was stirred for 2 h. The reaction mixture was purified by reverse phase chromatography to give compound 25 (20 mg, 0.018 mmol). MS calcd for C58H78N8O13, [M+H]=1095.6, found 1095.6.

To compound 25 (20 mg, 0.018 mmol) was added HCl (4N in Dioxane, 2 ml) at 0° C. The reaction mixture was stirred for 2 h at RT. The reaction mixture was concentrated to give the compound 26 (PS-0018; 18 mg, 0.018 mmol). MS calcd for C53H70N8O11, [M+H]=995.5, found 995.4.

To a solution of compound 16 (3.2 mg, 0.004 mmol) in 1 mL DMF was added compound 17 (1.9 mg, 0.006 mmol) and DIPEA (30 ul) at RT. The reaction mixture was stirred for 2 days. The reaction mixture was added slowly to HCl salt compound 26 (5 mg) with DIPEA (30 ul) in DMF (1 ml). After stirring for 5 h, the reaction mixture was purified by reverse phase chromatography and lyophilized to give the final compound 27 (PS-0024; 2 mg, 0.001 mmol). MS calcd for C92H126N14O22, [½M+H]=890.4, found 890.6.

### Example 1d

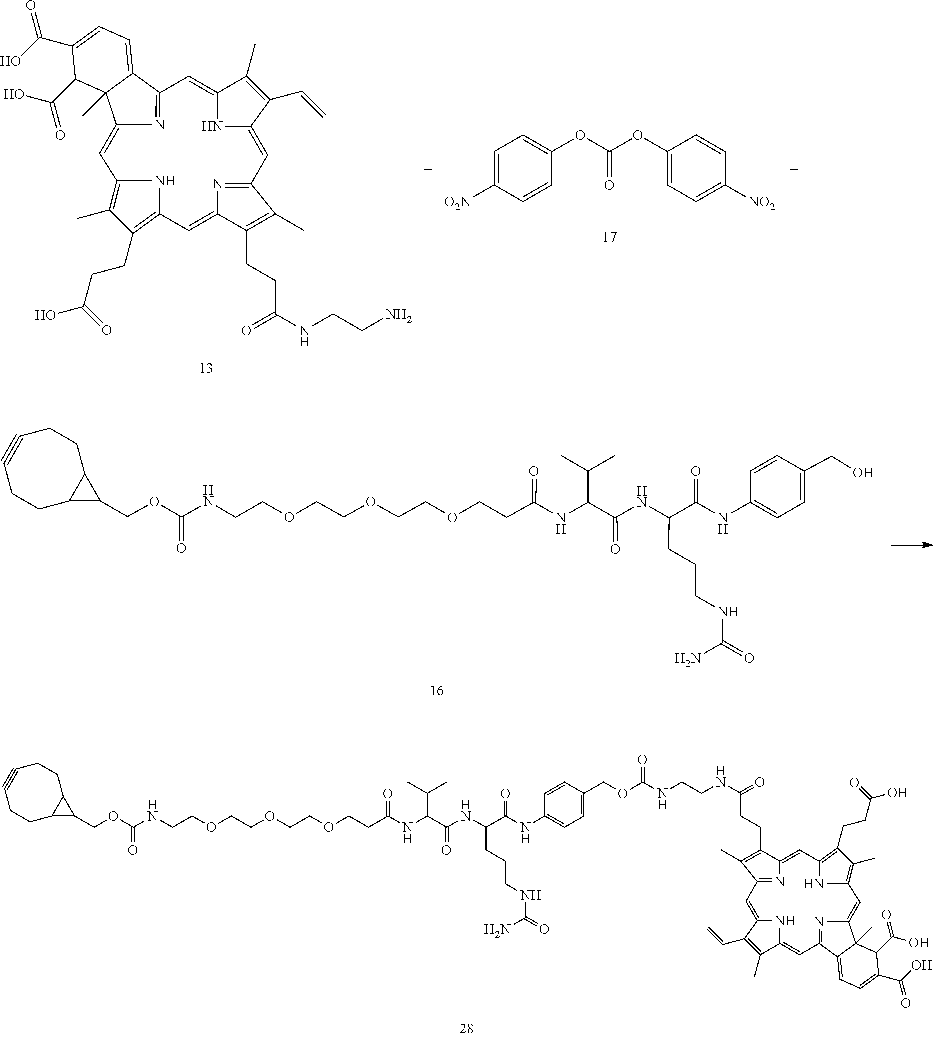 [SMILES: C=Cc1c(C)c2cc3nc(cc4[nH]c(cc5nc(cc1[nH]2)C(C)=C5CCC(=O)NCCN)c(CCC(=O)O)c4C)C1(C)C3=CC=C(C(=O)O)C1C(=O)O ; O=C(Oc1ccc([N+](=O)[O-])cc1)Oc1ccc([N+](=O)[O-])cc1 ; CC(C)C(NC(=O)CCOCCOCCOCCNC(=O)OCC1C2CCC#CCCC21)C(=O)NC(CCCNC(N)=O)C(=O)Nc1ccc(CO)cc1 ; C=Cc1c(C)c2cc3nc(cc4[nH]c(cc5nc(cc1[nH]2)C(C)=C5CCC(=O)NCCNC(=O)OCc1ccc(NC(=O)C(CCCNC(N)=O)NC(=O)C(NC(=O)CCOCCOCCOCCNC(=O)OCC2C5CCC#CCCC52)C(C)C)cc1)c(CCC(=O)O)c4C)C1(C)C3=CC=C(C(=O)O)C1C(=O)O]

To a solution of compound 16 (13 mg, 0.017 mmol) in 1.5 mL DMF was added compound 17 (6.5 mg, 0.021 mmol) and DIPEA (20 ul) at RT. The reaction mixture was stirred for 2 days. The reaction mixture was added slowly to HCl salt compound 13 (20 mg) with DIPEA (200 ul) in DMF (3 ml). After stirring for 5 h, the reaction mixture was purified by reverse phase chromatography and lyophilized to give the final compound 28 (PS-0019; 5 mg, 0.0033 mmol). MS calcd for C79H98N12O18, [½M+H]=752.4, found 752.4.

### Example 1e

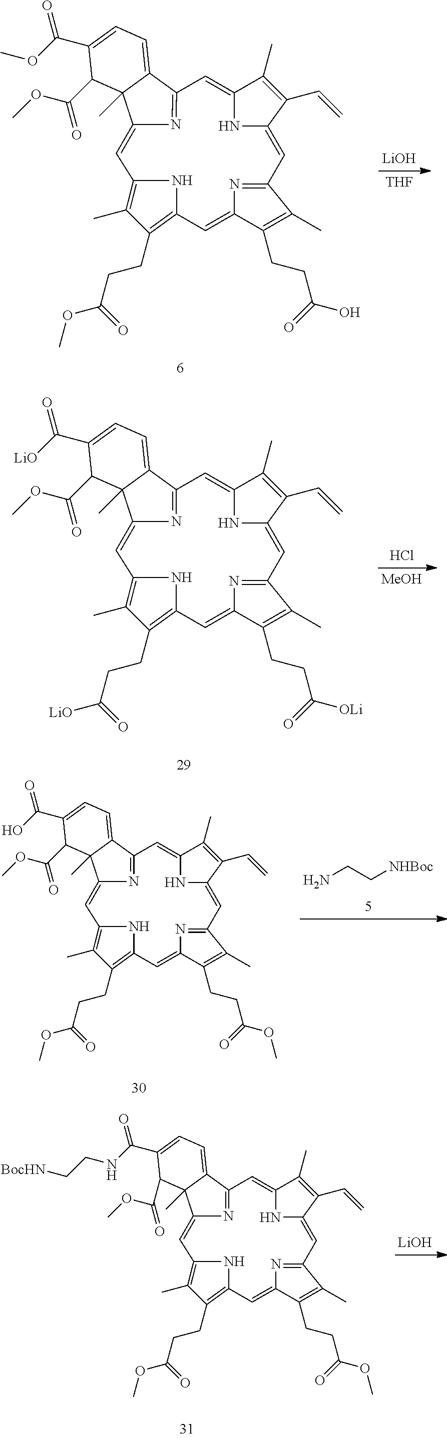

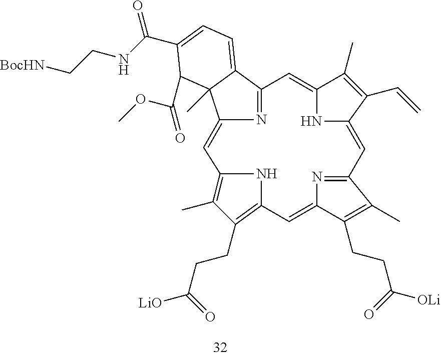

To a solution of verteporfin (compound 6; 70 mg, 0.1 mmol) in 20 mL MeOH was added LiGH (2N, 2.5 ml) at RT. The reaction mixture was stirred for 2 days at RT. The mixture was concentrated and purified by reverse phase chromatography to give the compound 29 (50 mg, 0.072 mmol). MS calcd for C39H38N4O₈, [M+H]=691.3, found 691.4.

To a solution of compound 29 (50 mg, 0.072 mmol) in 10 mL MeOH was added HCl (4N in Dioxane, 1 ml) at 0° C. The reaction mixture was stirred for 1 h at RT. The reaction mixture was concentrated to give the compound 30 (52 mg, 0.072 mmol). MS calcd for C41H42N4O8, [M+H]=719.3, found 719.3.

To a solution of compound 30 (48 mg, 0.07 mmol) in 4 ml DMF was added HATU (38 mg, 0.1 mmol) and DIPEA (35 ul, 0.20 mmol) at RT. The reaction mixture was stirred for 30 min and amine (compound 31; 12.8 mg, 0.08 mmol) was added. The mixture was stirred for 2 h. The reaction mixture was purified by reverse phase chromatography to give compound 32 (40 mg, 0.046 mmol). MS calcd for C48H56N6O9, [M+H]=861.4, found 861.4.

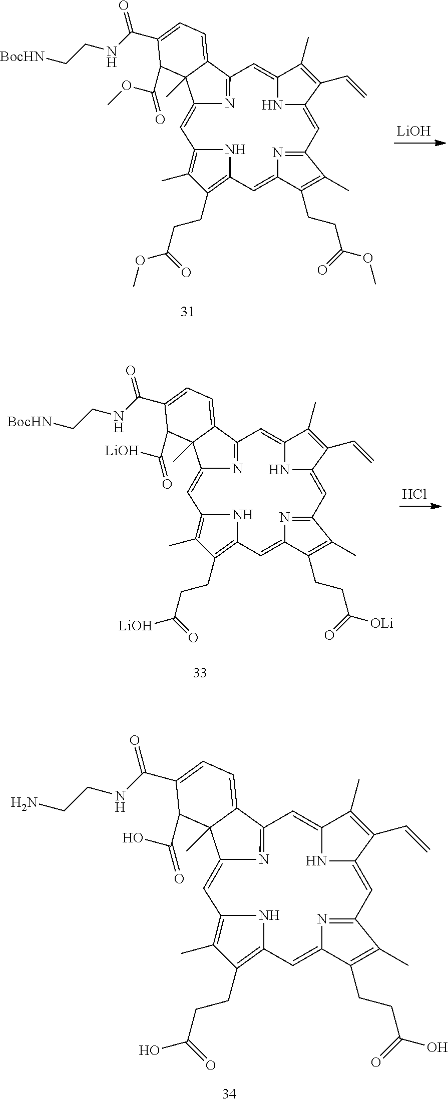

To a solution of compound 31 (45 mg, 0.052 mmol) in 5 mL THF was added LiGH (2N, 1.5 ml) at RT. The reaction mixture was stirred for 3 days at RT. THE was removed and the residue was purified by reverse phase chromatography to give the compound 33 (21 mg, 0.026 mmol). MS calcd for C45H50N6O9, [M+H]=819.4, found 819.4.

To the compound 33 (21 mg, 0.026 mmol) was added HCl (4N in Dioxane, 2 ml) at 0° C. The reaction mixture was stirred for 5 h at RT. The reaction mixture was concentrated to give the compound 34 (PS-0022; 20 mg, 0.026 mmol). MS calcd for C40H42N6O7, [M+H]=719.3, found 719.3.

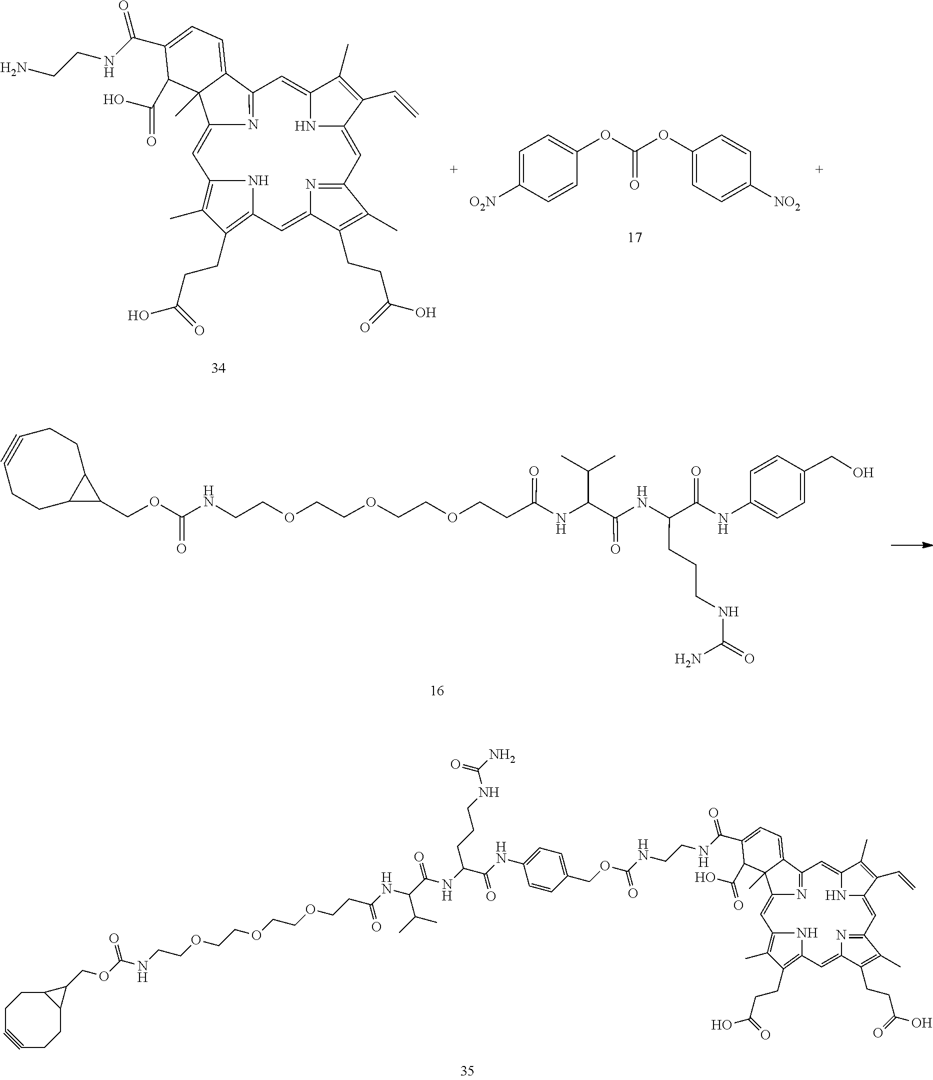 [SMILES: C=Cc1c(C)c2cc3nc(cc4[nH]c(cc5nc(cc1[nH]2)C(C)=C5CCC(=O)O)c(CCC(=O)O)c4C)C1(C)C3=CC=C(C(=O)NCCN)C1C(=O)O ; O=C(Oc1ccc([N+](=O)[O-])cc1)Oc1ccc([N+](=O)[O-])cc1 ; CC(C)C(NC(=O)CCOCCOCCOCCNC(=O)OCC1C2CCC#CCCC21)C(=O)NC(CCCNC(N)=O)C(=O)Nc1ccc(CO)cc1 ; C=Cc1c(C)c2cc3nc(cc4[nH]c(cc5nc(cc1[nH]2)C(C)=C5CCC(=O)O)c(CCC(=O)O)c4C)C1(C)C3=CC=C(C(=O)NCCNC(=O)OCc2ccc(NC(=O)C(CCCNC(N)=O)NC(=O)C(NC(=O)CCOCCOCCOCCNC(=O)OCC3C4CCC#CCCC43)C(C)C)cc2)C1C(=O)O]

To a solution of compound 16 (16 mg, 0.021 mmol) in 1.5 mL DMF was added compound 17 (7.7 mg, 0.025 mmol) and Et3N (20 ul) at RT. The reaction mixture was stirred for 24 h. The reaction mixture was added slowly to HCl salt compound 34 (20 mg) with Et3N (100 ul) in DMF (3 ml). After stirring for 5 h, the reaction mixture was purified by reverse phase chromatography and lyophilized to give the final compound 35 (PS-0023; 7 mg, 0.0046 mmol). MS calcd for C79H98N12O18, [½M+H]=752.4, found 752.4.

### Example 1f

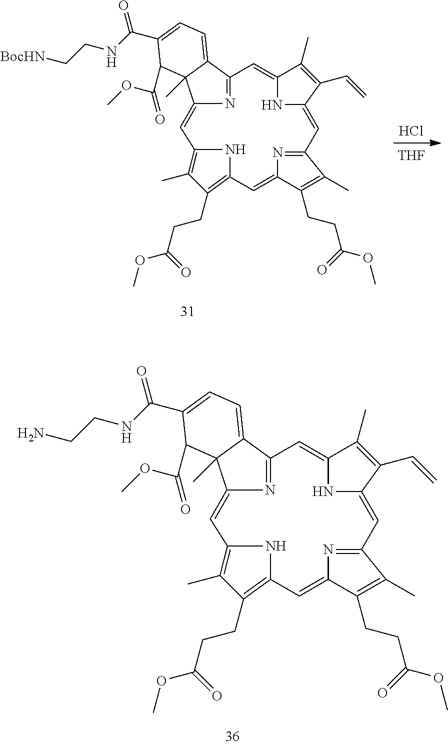 [SMILES: C=Cc1c(C)c2cc3nc(cc4[nH]c(cc5nc(cc1[nH]2)C(C)=C5CCC(=O)OC)c(CCC(=O)OC)c4C)C1(C)C3=CC=C(C(=O)NCCNC(=O)OC(C)(C)C)C1C(=O)OC ; Cl ; C=Cc1c(C)c2cc3nc(cc4[nH]c(cc5nc(cc1[nH]2)C(C)=C5CCC(=O)OC)c(CCC(=O)OC)c4C)C1(C)C3=CC=C(C(=O)NCCN)C1C(=O)OC ; "THF"]

To a solution of compound 31 (15 mg, 0.017 mmol) in 3 mL THF was added HCl (4N in Dioxane, 1 ml) at 0° C. The reaction mixture was stirred for 5 h at RT. The reaction mixture was concentrated to give the compound 36 (PS-0021; 15 mg, 0.017 mmol). MS calcd for C43H48N6O7, [M+H]=760.4.

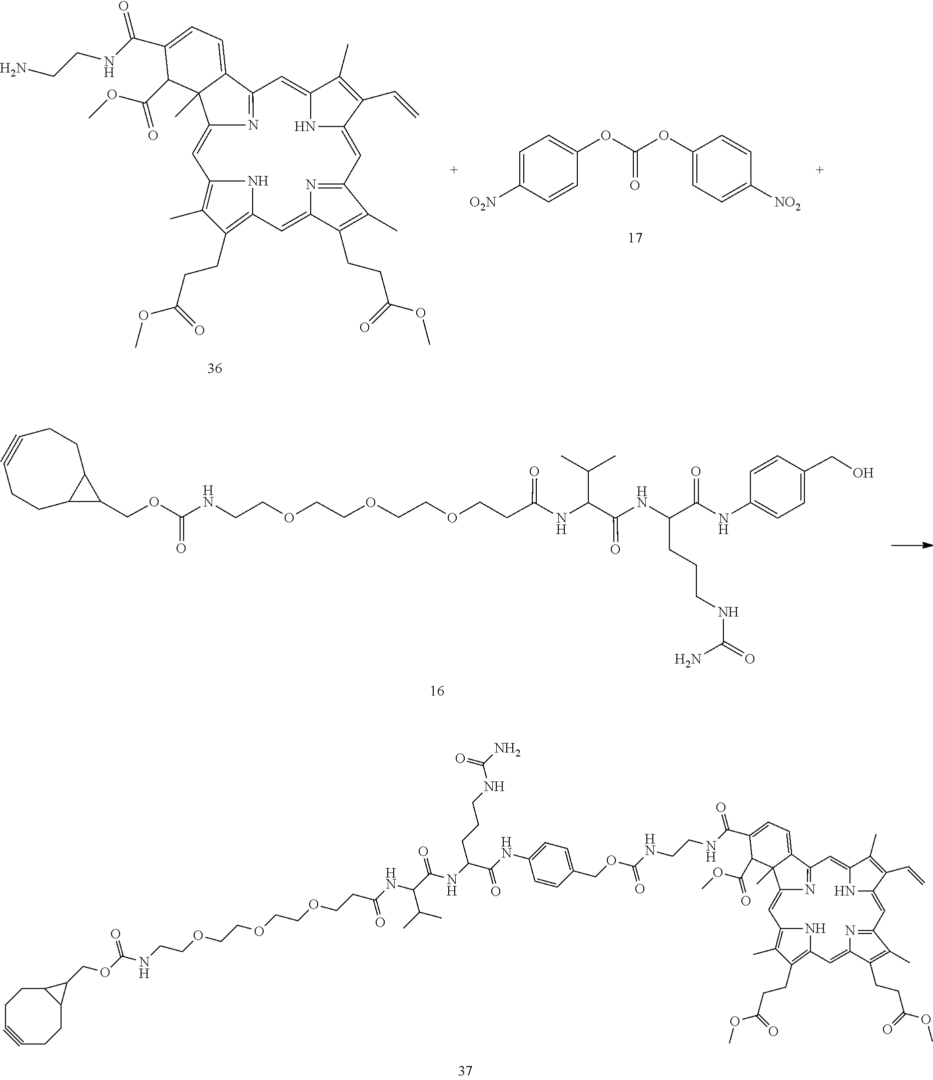 [SMILES: C=Cc1c(C)c2cc3nc(cc4[nH]c(cc5nc(cc1[nH]2)C(C)=C5CCC(=O)OC)c(CCC(=O)OC)c4C)C1(C)C3=CC=C(C(=O)NCCN)C1C(=O)OC ; O=C(Oc1ccc([N+](=O)[O-])cc1)Oc1ccc([N+](=O)[O-])cc1 ; CC(C)C(NC(=O)CCOCCOCCOCCNC(=O)OCC1C2CCC#CCCC21)C(=O)NC(CCCNC(N)=O)C(=O)Nc1ccc(CO)cc1 ; C=Cc1c(C)c2cc3nc(cc4[nH]c(cc5nc(cc1[nH]2)C(C)=C5CCC(=O)OC)c(CCC(=O)OC)c4C)C1(C)C3=CC=C(C(=O)NCCNC(=O)OCc2ccc(NC(=O)C(CCCNC(N)=O)NC(=O)C(NC(=O)CCOCCOCCOCCNC(=O)OCC3C4CCC#CCCC43)C(C)C)cc2)C1C(=O)OC]

To a solution of compound 16 (13 mg, 0.017 mmol) in 1 mL DMF was added compound 17 (6.5 mg, 0.021 mmol) and Et3N (20 ul) at RT. The reaction mixture was stirred for 24 h. The reaction mixture was added slowly to HCl salt compound 36 (15 mg) with Et3N (100 ul) in DMF (2 ml). After stirring for 5 h, the reaction mixture was purified by reverse phase chromatography and lyophilized to give the compound 37 (10 mg, 0.0065 mmol). MS calcd for C82H104N12O18, [½M+H]=773.4, found 773.4.

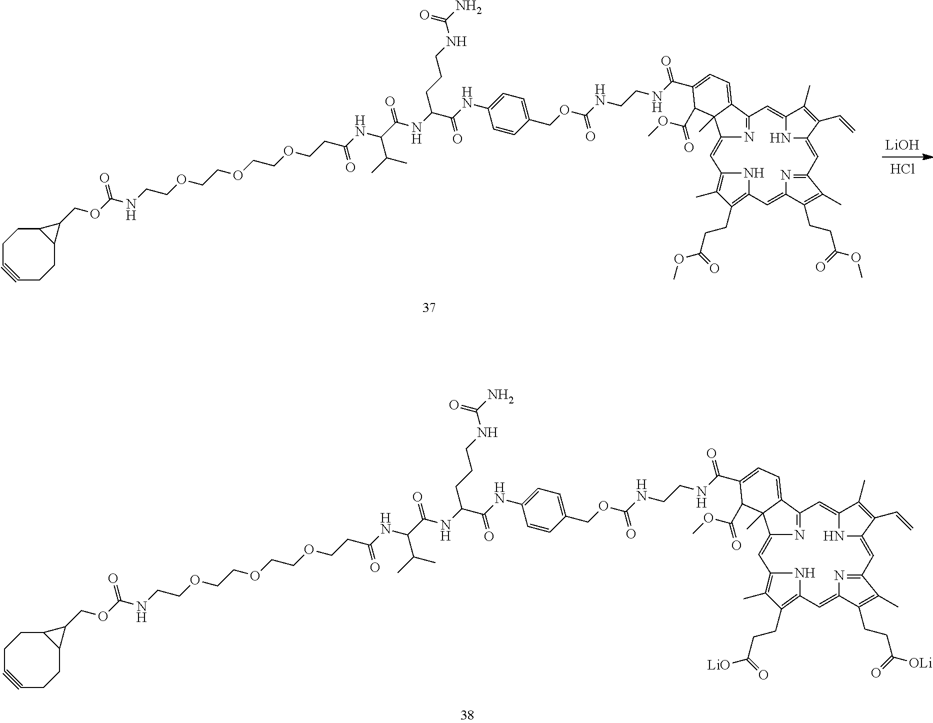

To a solution of compound 37 (10 mg, 0.0065 mmol) in 3 mL TH was added LiH (2N, 200 ul) at 0° C. The reaction mixture was stirred for 2 h at RT. The reaction mixture was purified by reverse phase chromatography to give the compound 38 (PS-0020; 6 mg, 0.005 mmol). MS calcd for C80H100N12O18, [½M+H]=759.4, found 759.4.

### Example 1g

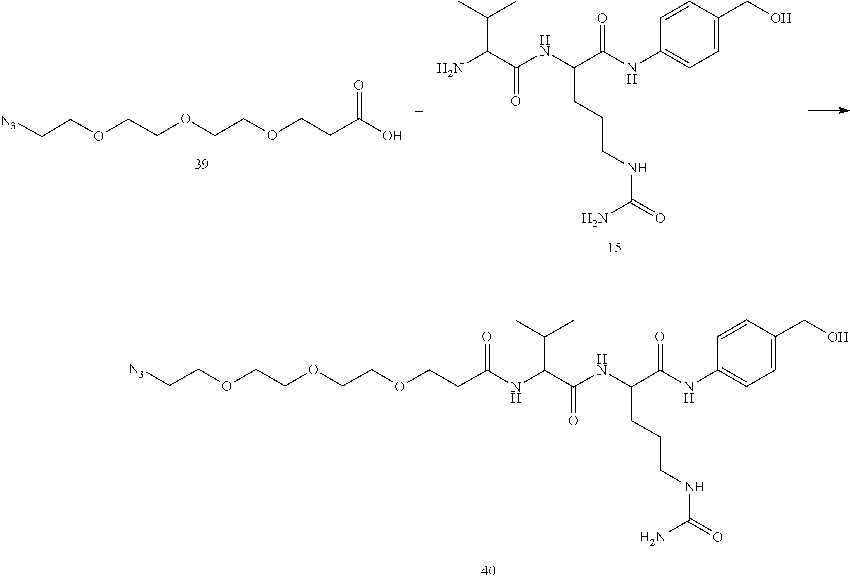 [SMILES: [N-]=[N+]=NCCOCCOCCOCCC(=O)O ; CC(C)C(N)C(=O)NC(CCCNC(N)=O)C(=O)Nc1ccc(CO)cc1 ; CC(C)C(NC(=O)CCOCCOCCOCCN=[N+]=[N-])C(=O)NC(CCCNC(N)=O)C(=O)Nc1ccc(CO)cc1]

To a solution of compound 39 (49.5 mg, 0.2 mmol) in 4 ml DMF was added HATU (80 mg, 0.21 mmol) and DIPEA (105 ul, 0.6 mmol) at RT. The reaction mixture was stirred for 10 min and Val-Cit-PAB-OH 15 (76 mg, 0.2 mmol) was added. The mixture was stirred for 2 h. The mixture was purified by reverse phase chromatography to give the compound 40 (70 mg, 0.11 mmol). MS cald for C27H44N8O8, [M+H]=609.3, found 609.4.

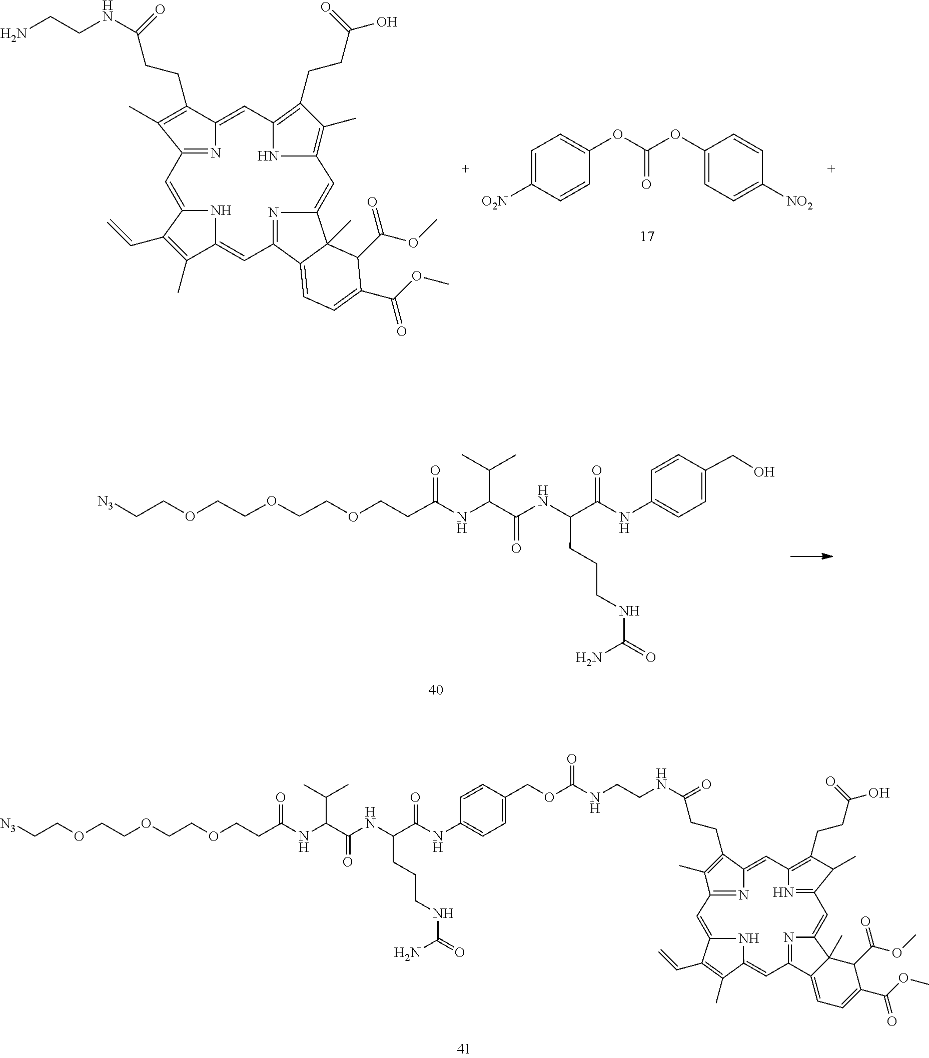 [SMILES: C=Cc1c(C)c2cc3nc(cc4[nH]c(cc5nc(cc1[nH]2)C(C)=C5CCC(=O)NCCN)c(CCC(=O)O)c4C)C1(C)C3=CC=C(C(=O)OC)C1C(=O)OC ; O=C(Oc1ccc([N+](=O)[O-])cc1)Oc1ccc([N+](=O)[O-])cc1 ; CC(C)C(NC(=O)CCOCCOCCOCCN=[N+]=[N-])C(=O)NC(CCCNC(N)=O)C(=O)Nc1ccc(CO)cc1 ; C=Cc1c(C)/c2[nH]/c1=C\C1=NC(=C\C3=C(CCC(=O)O)C(C)C(=N3)/C=C3N=C(\C=2)C2=CC=C(C(=O)OC)C(C(=O)OC)C2\3C)/C(CCC(=O)NCCNC(=O)OCc2ccc(NC(=O)C(CCCNC(N)=O)NC(=O)C(NC(=O)CCOCCOCCOCCN=[N+]=[N-])C(C)C)cc2)=C1C]

To a solution of compound 40 (78 mg, 0.13 mmol) in 5 mL DMF was added compound 17 (44 mg, 0.14 mmol) at RT. The reaction mixture was stirred for overnight. The reaction mixture was added slowly to HCl salt 9 (120 mg) with DIPEA (300 ul) in DCM (20 ml). After stirring for 1 hr, DCM was removed under reduced pressure and purified by reverse phase chromatography and lyophilized to give the desired compound 41 (PS-0025; 130 mg, 0.094 mmol). MS calcd for C70H88N14016, [M+H]=1381.6, found 1381.6. 1H NMR (500 MHz, DMSO-d6): δ 10.44 (d, 1H, J=15), 10.10 (d, 1H, J=15), 9.86 (m, 1H), 9.64 (s, 1H), 9.27 (d, 1H, J=15), 8.42 (dd, 1H, J=15, J=10), 8.29 (m, 1H), 7.89 (m, 1H), 7.76 (s, 3H), 7.63 (m, 2H), 7.31 (m, 2H), 6.48 (d, 1H, J=15), 6.20 (m, 1H), 6.01 (m, 1H), 5.38 (s, 2H), 5.23 (s, 1H), 4.96 (s, 1H), 4.42-4.36 (m, 2H), 4.24-4.05 (m, 4H), 4.03-3.93 (m, 2H), 3.90 (s, 3H), 3.61-3.20 (m, 24H), 3.05-2.85 (m, 7H), 2.80-2.65 (m, 3H), 2.45-2.30 (m, 2H), 2.00-1.90 (m, 1H), 1.76 (s, 3H), 1.70-1.65 (m, 1H), 1.64-1.52 (m, 1H), 1.45-1.30 (m, 3H), 0.84-0.80 (m, 8H), −2.54 (m, 2H).

### Example 1h

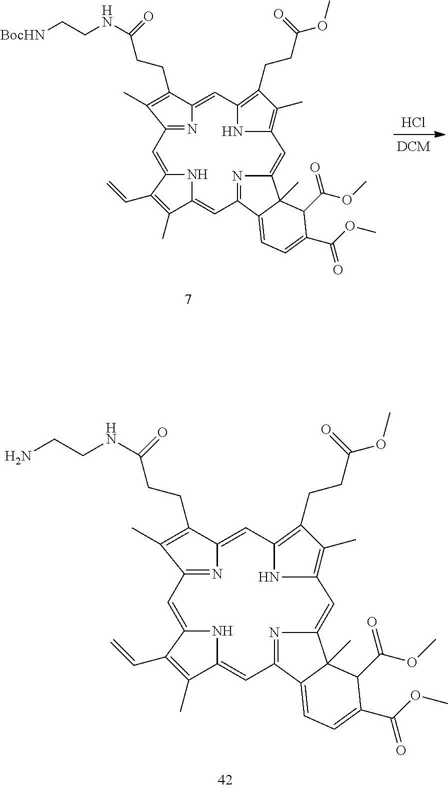 [SMILES: C=Cc1c(C)c2cc3nc(cc4[nH]c(cc5nc(cc1[nH]2)C(C)=C5CCC(=O)NCCNC(=O)OC(C)(C)C)c(CCC(=O)OC)c4C)C1(C)C3=CC=C(C(=O)OC)C1C(=O)OC ; Cl ; C=Cc1c(C)c2cc3nc(cc4[nH]c(cc5nc(cc1[nH]2)C(C)=C5CCC(=O)NCCN)c(CCC(=O)OC)c4C)C1(C)C3=CC=C(C(=O)OC)C1C(=O)OC]

To a solution of compound 7 (12 mg) in 3 mL DCM was added HCl (4N in Dioxane, 1 ml) at 0° C. The reaction mixture was stirred for 1 h at RT. The reaction mixture was concentrated to give the compound 42 (PS-0030). MS calcd for C43H48N6O7, [M+H]=761.4, found 761.4.

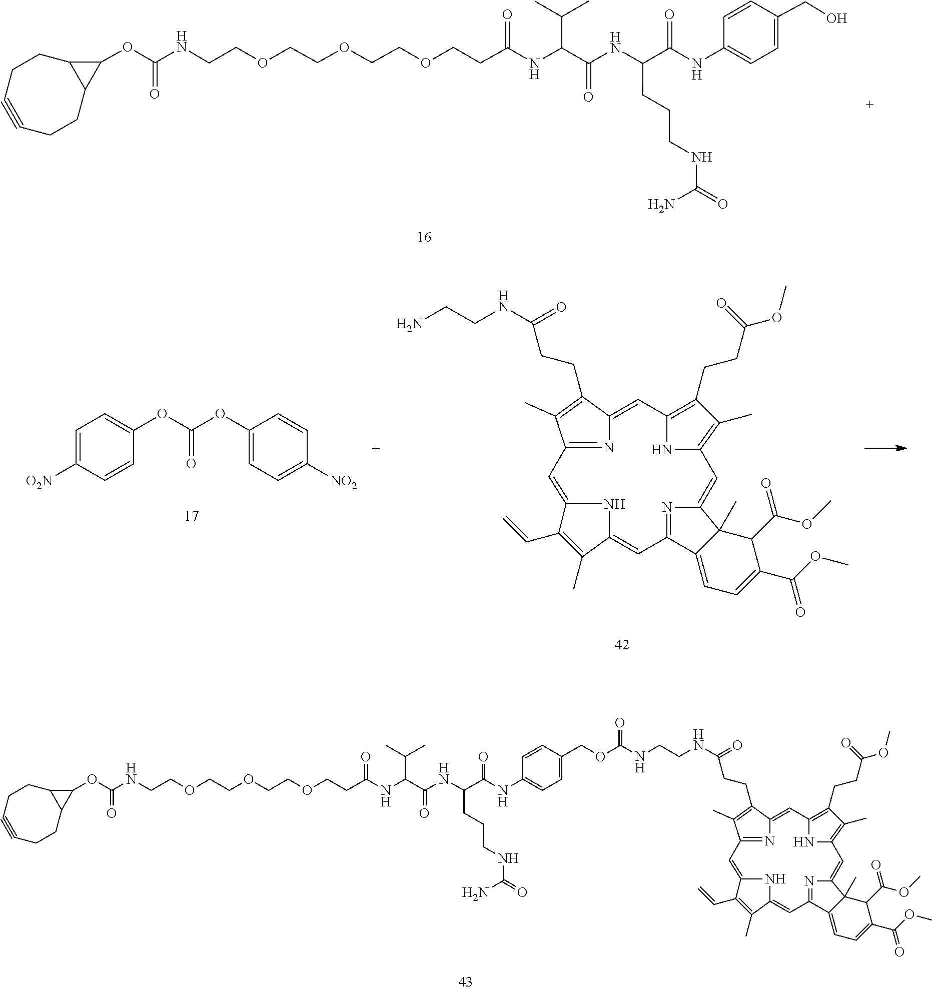 [SMILES: CC(C)C(NC(=O)CCOCCOCCOCCNC(=O)OC1C2CCC#CCCC21)C(=O)NC(CCCNC(N)=O)C(=O)Nc1ccc(CO)cc1 ; O=C(Oc1ccc([N+](=O)[O-])cc1)Oc1ccc([N+](=O)[O-])cc1 ; C=Cc1c(C)c2cc3nc(cc4[nH]c(cc5nc(cc1[nH]2)C(C)=C5CCC(=O)NCCN)c(CCC(=O)OC)c4C)C1(C)C3=CC=C(C(=O)OC)C1C(=O)OC ; C=Cc1c(C)c2cc3nc(cc4[nH]c(cc5nc(cc1[nH]2)C(C)=C5CCC(=O)NCCNC(=O)OCc1ccc(NC(=O)C(CCCNC(N)=O)NC(=O)C(NC(=O)CCOCCOCCOCCNC(=O)OC2C5CCC#CCCC52)C(C)C)cc1)c(CCC(=O)OC)c4C)C1(C)C3=CC=C(C(=O)OC)C1C(=O)OC]

To a solution of compound 16 (11 mg) in 1 mL DMF was added compound 17 (6.5 mg) and DIPEA (5 ul) at RT. The reaction mixture was stirred for overnight. The reaction mixture was added slowly to HCl salt 42 (10 mg) with DIPEA (30 ul) in DCM (2 ml). After stirring for 1 hr., DCM was removed under reduced pressure and purified by reverse phase chromatography twice to give the final compound 43 (PS-0026, 2 mg). MS calcd for C82H104N12O18, [½M+H]=773.4, found 773.4.

### Example 1i

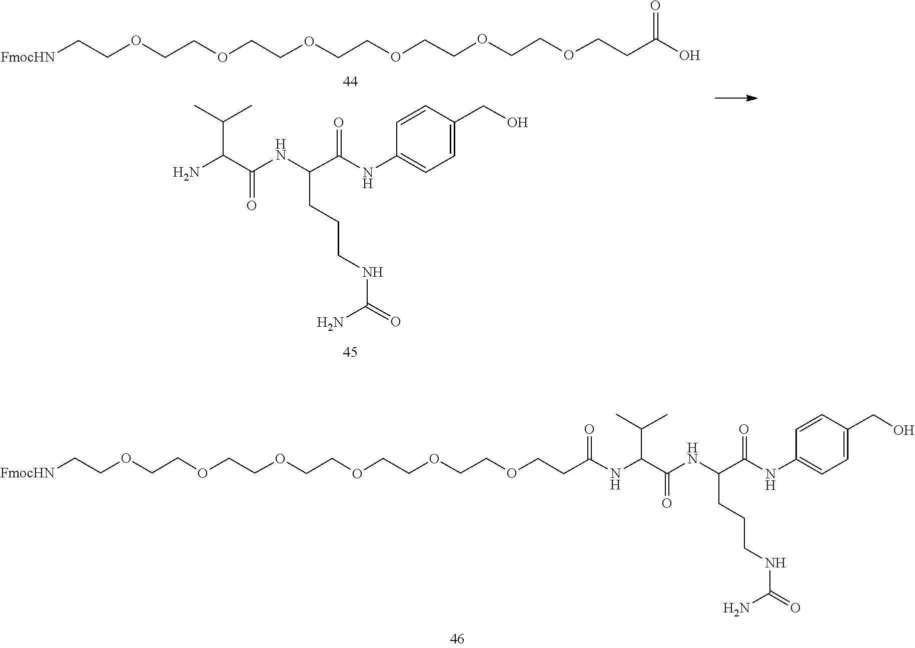

To a solution of Fmoc-PEG6-acid 44 (155.0 mg, 0.27 mmol) in 5 ml DMF was added HATU (110 mg, 0.29 mmol) and DIPEA (94 ul, 0.54 mmol) at RT. The reaction mixture was stirred for 10 min and Val-Cit-PAB-OH 45 (102 mg, 0.27 mmol) was added. The mixture was stirred for 2 h. The mixture was purified by reverse phase chromatography to give the compound 46 (170 mg, 0.18 mmol). MS calcd for C48H68N6O13, [M+H]=937.4, found 937.4.

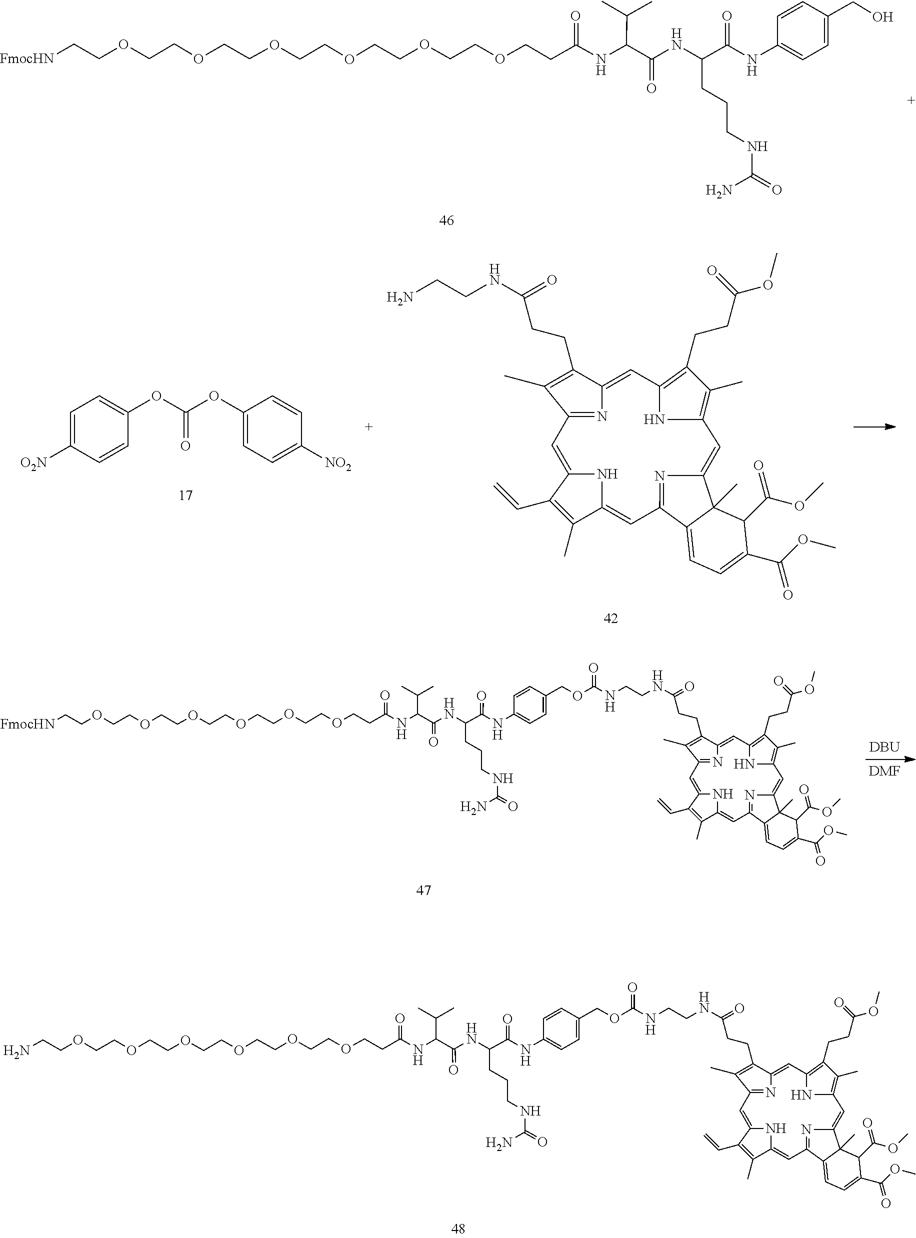

To a solution of compound 46 (30 mg, 0.032 mmol) in 5 mL DMF was added compound 17 (10 mg, 0.033 mmol) and DIPEA (6.5 ul, 0.037 mmol) at RT. The reaction mixture was stirred for overnight. The reaction mixture was added slowly to HCl salt 42 (30 mg) with DIPEA (30 ul) in DMF (4.0 ml). After stirring for 5 h, DCM was removed under reduced pressure and purified by reverse phase chromatography and lyophilized to give the desired compound 47 (28 mg, 0.016 mmol). MS calcd for C92H114N12O21, ½[M+H]=862.4, found ½[M+H]=862.4.

To a solution of compound 47 (28 mg, 0.016 mmol) in 2 mL DMF was added DBU (50 ul) at RT. The reaction mixture was stirred for 1 h. The reaction mixture was purified by reverse phase chromatography to give compound 48 (PS-0028, 10 mg, 0.007 mmol). MS calcd for C77H104N12O19, ½[M+H]=751.4, found ½[M+H]=751.4.

### Example 1j

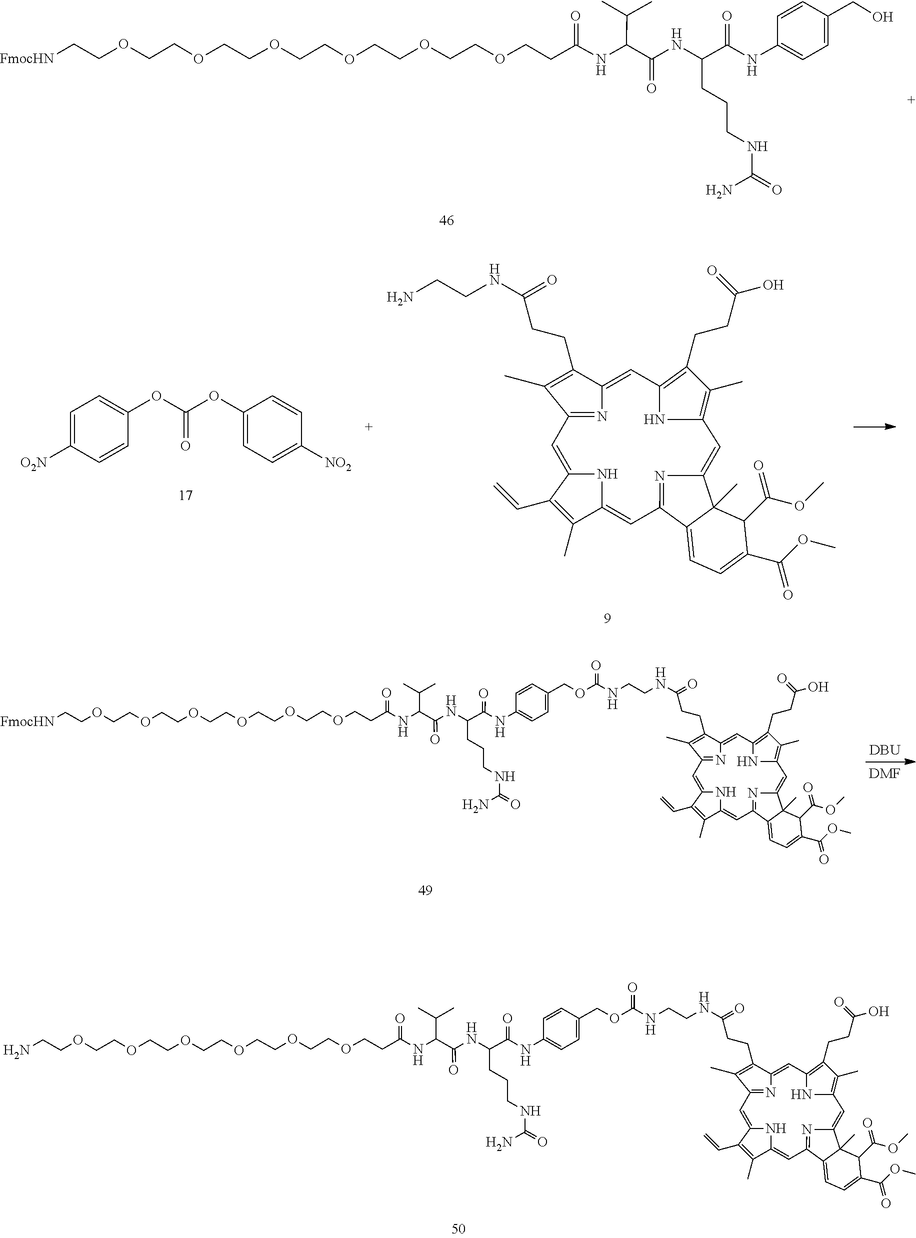

To a solution of compound 46 (30 mg, 0.032 mmol) in 5 mL DMF was added compound 17 (10 mg, 0.033 mmol) and DIPEA (6.5 ul, 0.037 mmol) at RT. The reaction mixture was stirred for overnight. The reaction mixture was added slowly to HCl salt 9 (30 mg) with DIPEA (30 ul) in DCM (4.0 ml). After stirring for 5 h, DCM was removed under reduced pressure and purified by reverse phase chromatography to give compound 49 (26 mg, 0.015 mmol). MS calcd for C₉₁H112N12O21, ½[M+H]=855.4, found ½[M+H]=855.4.

To a solution of compound 49 (26 mg, 0.015 mmol) in 2 mL DMF was added DBU (50 ul) at RT. The reaction mixture was stirred for 1 h. The reaction mixture was purified by reverse phase chromatography to give compound 50 (PS-0027, 14 mg, 0.01 mmol). MS calcd for C76H102N12O19, ½[M+H]=744.4, found ½[M+H]=744.3.

### Example 1k

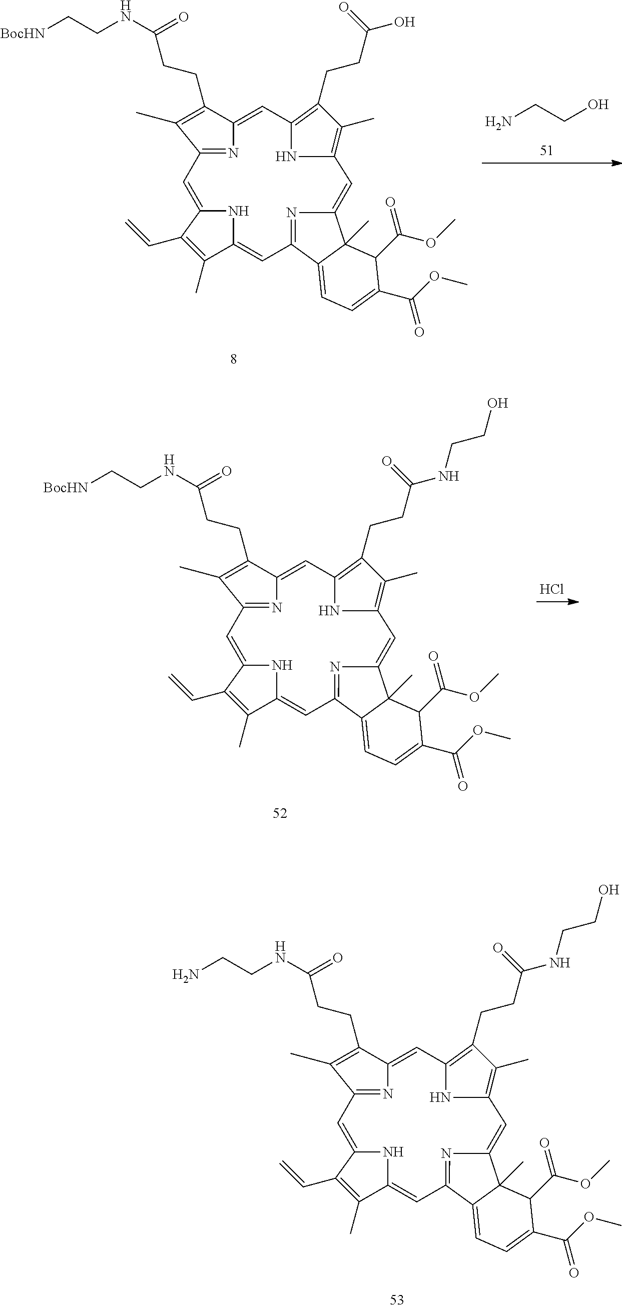

To a solution of compound 8 (35.0 mg, 0.041 mmol) in 2.0 ml DMF was added HATU (17 mg, 0.045 mmol) and DIPEA (14 ul, 0.082 mmol) at RT. The reaction mixture was stirred for 10 min and compound 51 (4 ul, 0.06 mmol) was added. The mixture was stirred for 3 h. The mixture was purified by reverse phase chromatography to give the compound 52 (25 mg, 0.028 mmol). MS calcd for C49H59N7O9, [M+H]=890.4, found 890.4.

To a solution of compound 52 (25 mg, 0.028 mmol) in 3.0 mL DCM was added HCl (4N in Dioxane, 0.5 ml) at 0° C. The reaction mixture was stirred for 3 h at RT. The reaction mixture was concentrated to give the compound 53 (PS-0029, 20 mg, 0.028 mmol). MS calcd for C44H51N7O7, [M+H]=790.4, found 790.4.

### Example 2. Assembly of Photosensitizer Antibody Conjugates (PACs)

The concentrations of the peptides and proteins were determined using UV absorption at 280 nm and extinction coefficients based on amino acid sequences. All aqueous solutions were prepared using Milli-Q water. All cell culture methods were performed with aseptic technique in a biosafety cabinet.

### Non-Specific Conjugation: Amine Reactive Acylation Reaction to Construct Antibody Conjugates

The reaction contained 100 mM phosphate (pH 7.4), a bioconjugation handle (1.67 mM NHS-PEG₆-azide for Panitumumab or 2 mM NHS-PEG₄-BCN for anti-CD2) and antibody (6.7 μM panitumumab or 6.7 μM anti-CD2) and incubated at 22.5° C. for 16-18 h. The reaction was then quenched by 2 mM Tris (pH 8). To remove excess unreacted reagents, each reaction mixture was desalted using 50 kDa Amicon MWCO centrifugal filters into 100 mM Tris (pH 8) prior to the cycloaddition reaction.

### Strain Promoted Cycloaddition Click Reaction to Introduce the Photosensitizers

The reaction contained 100 mM Tris (pH 8), photosensitizer (64 μM of PS-0002 for panitumumab or 42 μM of PS-0025 for anti-CD2), antibody (6.4 μM panitumumab or 4.2 μM anti-CD2; acylated as aforementioned), 30% dimethylacetamide (DMA) and incubated at 22.5° C. for 16-18 h. To remove excess unreacted reagents, each reaction mixture was buffer exchanged into 100 mM Tris (pH 8) and 30% DMA using size exclusion chromatography resins (Zeba™ Spin Desalting Columns, ThermoFisher). Then, this mixture was desalted using 30 kDa Amicon MWCO centrifugal filters into 100 mM Tris (pH 8) and 5% DMSO prior to analysis.

### Characterization of the Conjugates by SDS-PAGE

Sodium dodecyl sulfate-polyacrylamide gel electrophoresis (SDS-PAGE) was performed using a mini-gel system (Thermo Fisher, A25977). First, the reaction mixture was incubated with 6×SDS Sample Buffer (Thermo Fisher, J61337AD) at 80° C. for 5 min.

Second, the samples were loaded into 12% tris-glycine precast protein gels (Thermo Fisher, XP00120BOX). PageRuler™ Prestained Protein Ladder (Thermo Fisher, 26617) were used for mass calibration. Electrophoresis was then performed at 200 V for 30 min. The gel was stained by Coomassie dye (Thermo Fisher, GelCode™ Blue Safe Protein Stain, 24594) and then destained using Milli-Q water. The gels were imaged using an iBright FL1000 Imaging system (Thermo Fisher Scientific).

### Strain Promoted Cycloaddition Click Reaction to Introduce the Photosensitizers

Panitumumab PACs for structure-activity relationship (SAR) studies. The reaction contained 100 mM Tris (pH 8), photosensitizer (34.7 mM of either PS-0011, PS-0020, PS-0019, PS-0002, PS-0003 or PS-0004), antibody (3.47 mM panitumumab; acylated as aforementioned), 30% dimethylacetamide (DMA), 10 mM sodium decanoate (NaDec) and incubated at 22.5° C. for 16-18 h. For cleavable VertDA PAC conjugates only, the reaction contained 100 mM Tris (pH 8), 66.7 uM of PS-0020, 6.7 uM panitumumab (acylated as aforementioned), 30% dimethylacetamide (DMA), 5 mM sodium decanoate (NaDec) and incubated at 22.5° C. for 16-18 h. To remove excess unreacted reagents, each reaction mixture was buffer exchanged into 100 mM Tris (pH 8), 30% DMA and 10 mM NaDec using size exclusion chromatography resins (Zeba™ Spin Desalting Columns, ThermoFisher). Then, this mixture was desalted using 30 kDa Amicon MWCO centrifugal filters into 100 mM Tris (pH 8), 5% DMSO and 5 mM NaDec prior to analysis.

### Example 3. Measurement of Reactive Oxygen Species (ROS) Generation

Singlet Oxygen Sensor Green (SOSG, Invitrogen, S36002) was reconstituted according to manufacturer instructions and was further diluted to 2 μM in fresh PBS. Photosensitizers (PSs) were also freshly prepared by diluting to 2 μM in fresh PBS. 50 μL of SOSG and 50 μL of PS were combined in black-walled 96-well plates. Samples were then irradiated with 690 nm light from an LED source for 0, 10, or 50 J/cm² total light dose. The fluorescence of SOSG (Ex/Em 500/540 nm) in each sample was then measured using a fluorometer (SpectraMax iD5). The total singlet oxygen generated is proportional to the fold-change in SOSG fluorescence, ΔF, calculated as the ratio of the irradiated (10 or 50 J/cm²) to non-irradiated (0 J/cm²) samples.

### Example 4. Cell Viability Assessment

A-431 cells were plated in 96-well plates at 2000 cells per well and incubated at 37° C. for 72 h. Cell media was then replaced with media containing the Photosensitizer-Antibody Conjugates (PAC) at various concentrations and incubated for 24 h at 37° C., after which the media was replaced with fresh media. Plates were immediately irradiated with 690 nm light from an LED source, applying various light doses (0, 3, 10, 20, 30, 40, or 50 J/cm²). The cells were incubated further for 72 h and viability was assessed using CellTiterGlo 2.0 viability detection kit (Promega, G9241).

Jurkat cells were plated in 96-well plates at 20000 cells per well with media containing PAC at various concentrations and incubated for 48 hours at 37° C. Plates were then irradiated with 690 nm light from an LED source, applying various light doses (0, 3, 10, 20, 30, 40, or 50 J/cm²). The cells were incubated further 72 hours and viability was assessed using CellTiterGlo 2.0 viability kit.

HH cells were plated in 96-well plates at 20000 cells per well with media containing PAC at various concentrations and incubated for 24 hours at 37° C. Plates were then irradiated with 100 J/cm² of 690 nm light from an LED source. The cells were incubated a further 24 hours and viability was assessed using CellTiterGlo 2.0 viability kit.

What is claimed is:

1. A compound represented by the following formula: 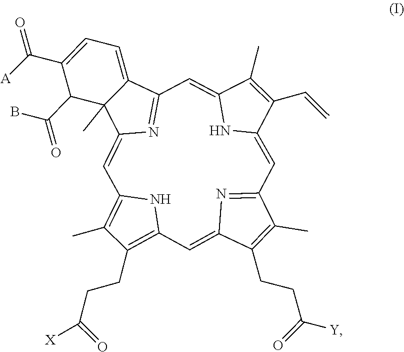
or a pharmaceutically acceptable salt thereof, wherein:
A, B, X and Y are each independently —OH, —OC₁₋₄alkyl, —N(R¹⁰⁰)(R¹⁰¹), or —Z-L^{P}-ker, provided at least one of A, B, X and Y is —N(R¹⁰⁰)(R¹⁰¹), —Z-L^{P}-NH₂, or —Z-L^{P}-ker, and provided at most one of A, B, X and Y is —Z-L^{P}-NH₂ or —Z-L^{P}-ker;
R¹⁰⁰ is H or C₁₋₃alkyl;
R¹⁰¹ is H, C₁₋₃alkyl or —(CH₂—CH₂—O)ₙᵣR¹⁰²;
R¹⁰² is H or Me;
nr is an integer from 1 to 16;
Z is —O— or —NR¹—;
R¹ is H or C₁₋₃alkyl;
L^{P} is a spacer;
ker is a reactive group.

2. The compound of claim 1, or a pharmaceutically acceptable salt thereof, wherein:
a) A and B are —OCH₃; and one of X and Y is —OCH₃ and the other one of X and Y is —Z-L^{P}-ker;
b) A and B are —OCH₃; and one of X and Y is —OH and the other one of X and Y is —Z-L^{P}-ker;
c) one of A and B is —OCH₃, and the other is —OH; and one of X and Y is —OH and the other one of X and Y is —Z-L^{P}-ker;
d) A and B are —OH; and one of X and Y is —OH and the other one of X and Y is —Z-L^{P}-ker;
e) one of A and B is —OH and the other one of A and B is —OH or —OCH₃; and X and Y are both —NH(CH₂—CH₂—O)ₙᵣR¹⁰², wherein R¹⁰² is H or Me; and nr is an integer from 2 to 8; or
f) one of A and B is —Z-L^{P}-ker and the other one of A and B is —OH or —OCH₃; and X and Y are both —NH(CH₂—CH₂—O)ₙᵣR¹⁰², wherein R¹⁰² is H or Me; and nr is an integer from 2 to 8.

3. The compound of claim 1, or a pharmaceutically acceptable salt thereof, wherein the compound is
(i) represented by the following formula: 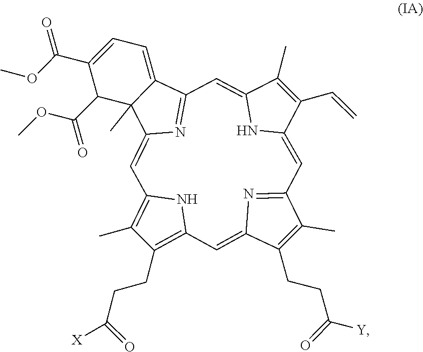
or a pharmaceutically acceptable salt thereof, wherein one of X and Y is —Z-L^{P}-ker and other is —OH; or
(ii) represented by the following formula: 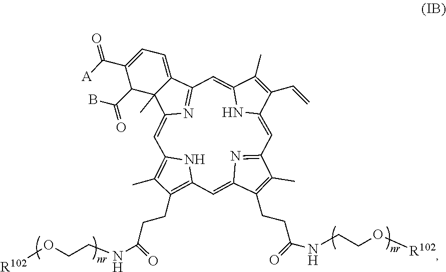
or a pharmaceutically acceptable salt thereof, wherein one of A and B is —OH or —Z-L^{P}-ker and the other is —OH or —OCH₃; R¹⁰² is H or Me; and nr is an integer from 2 to 8.

4. The compound of any one of claims 1-3, or a pharmaceutically acceptable salt thereof, wherein:
(i) ker is a reactive group selected from: a maleimide group, a thiol group, a cyclooctyne group, an azido group, a hydrazide group, a tetrazine group, cyclooctene group, a ketone group, and an aldehyde group; or
(ii) ker is 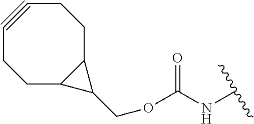 [SMILES: *NC(=O)OCC1C2CCC#CCCC21]
or —N₃.

5. The compound of any one of claims 1-4, or a pharmaceutically acceptable salt thereof, wherein:
(i) Z is —NH—; or
(ii) Z is —O—.

6. The compound of any one of claims 1-5, or a pharmaceutically acceptable salt thereof, wherein:
(i) L^{P} is a non-cleavable spacer, optionally, wherein:
(a) L^{P} comprises a hydrophilic moiety;
(b) L^{P} comprises a PEG moiety;
(c) L^{P} is *—CH₂CH₂—(OCH₂CH₂)ₘ—, wherein * is the site connected to ker, and m is an integer from 0 to 30, optionally, wherein m is an integer from 2 to 16, from 2 to 10, from 2 to 8, or from 3 to 5; or m is 2, 3, 4, 5, 6, 7 or 8; or
(d) L^{P} is *—CH₂CH₂—(OCH₂CH₂)₄—;
(ii) L^{P} is a cleavable spacer, optionally, wherein:
(a) the cleavable spacer comprises an enzyme labile group, a pH labile group, a disulfide group, a ROS cleavable group, a photocleavable group, optionally, wherein:
(1) the enzyme labile group is a group cleavable by an esterase, a peptidase, an aminopeptidase, a β-galactosidase, a β-glucuronidase, a carboxylesterase, a caspase, a diaphorase, a histone deacetylase, a legumain, or matrix metalloproteinase; or
(2) the enzyme labile group is a group cleavable by cathepsin B;
(ii) the spacer represented by L^{P} comprises a peptide and a self-immolative group; or
(iii) the spacer L^{P} is presented by the following formula: 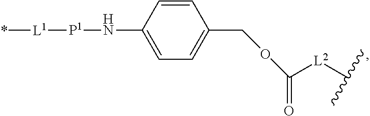
wherein:
*- represents a bond to the reactive group represented by ker; 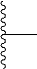
represents a bond to the group Z;
L¹ and L² are each independently a connecting spacer;
P¹ is a peptide comprising 2 to 5 amino acid residues, optionally, wherein:
(a1) P¹ is a peptide selected from Phe-Arg-Arg-Gly, Glu-Val-Cit, Val-Cit, Cit-Val, Gly-Gly-Phe, Val-Ala, and Ala-Val; or
P¹ is: 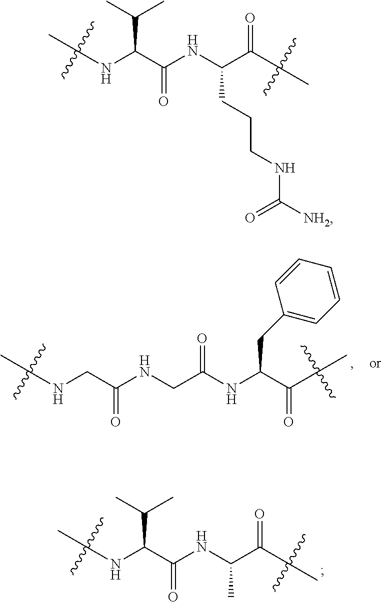
and/or
(a2) L¹ comprises a hydrophilic moiety;
L¹ comprises a PEG moiety;
L¹ is *—CH₂—CH₂—(OCH₂CH₂)ₙ—C(═O)—**; n is an integer from 0 to 30; *-represents a bond to the reactive group represented by ker; and **- represents a bond to P¹, optionally, wherein n is an integer from 2 to 16, from 2 to 10, from 2 to 8, or from 3 to 5; or n is 2, 3, 4, 5, 6, 7 or 8; or
L¹ is *—CH₂—CH₂—(OCH₂CH₂)₃—C(═O)—**; and/or
(a3) L² is —NH—C₁₋₆alkyl-**, wherein ** is the site connected to Z; or
L² is —NH—CH₂CH₂—**; wherein ** is the site connected to Z.

7. The compound of claim 1, or a pharmaceutically acceptable salt thereof, wherein the compound is: 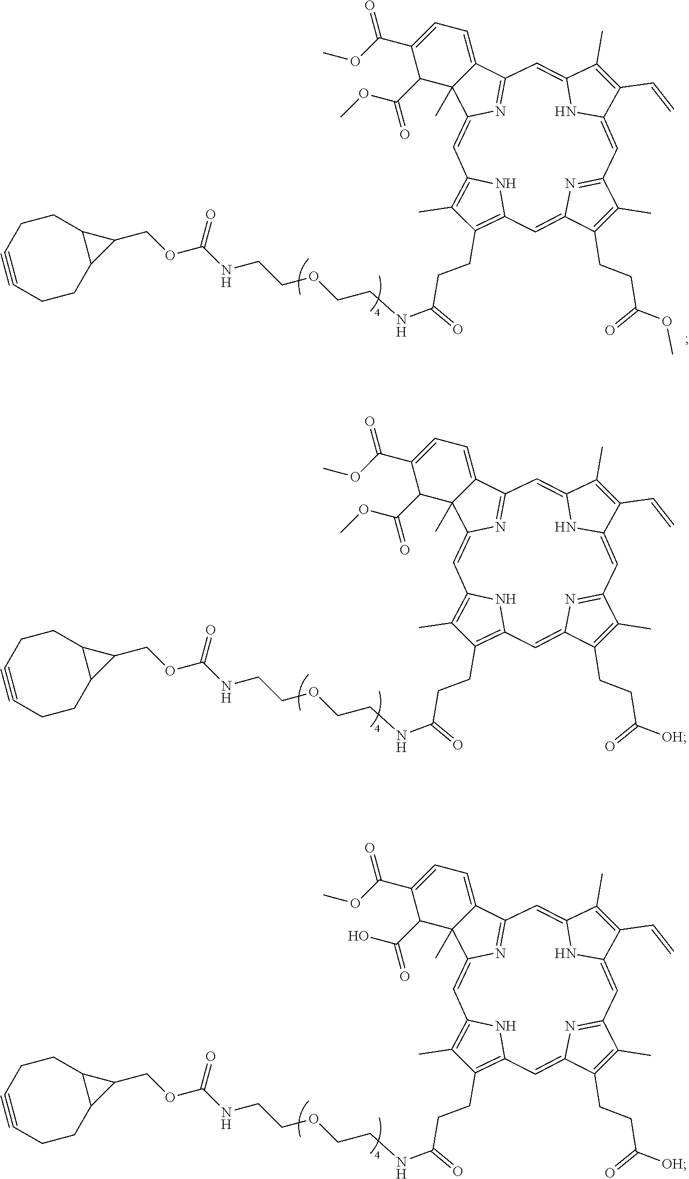 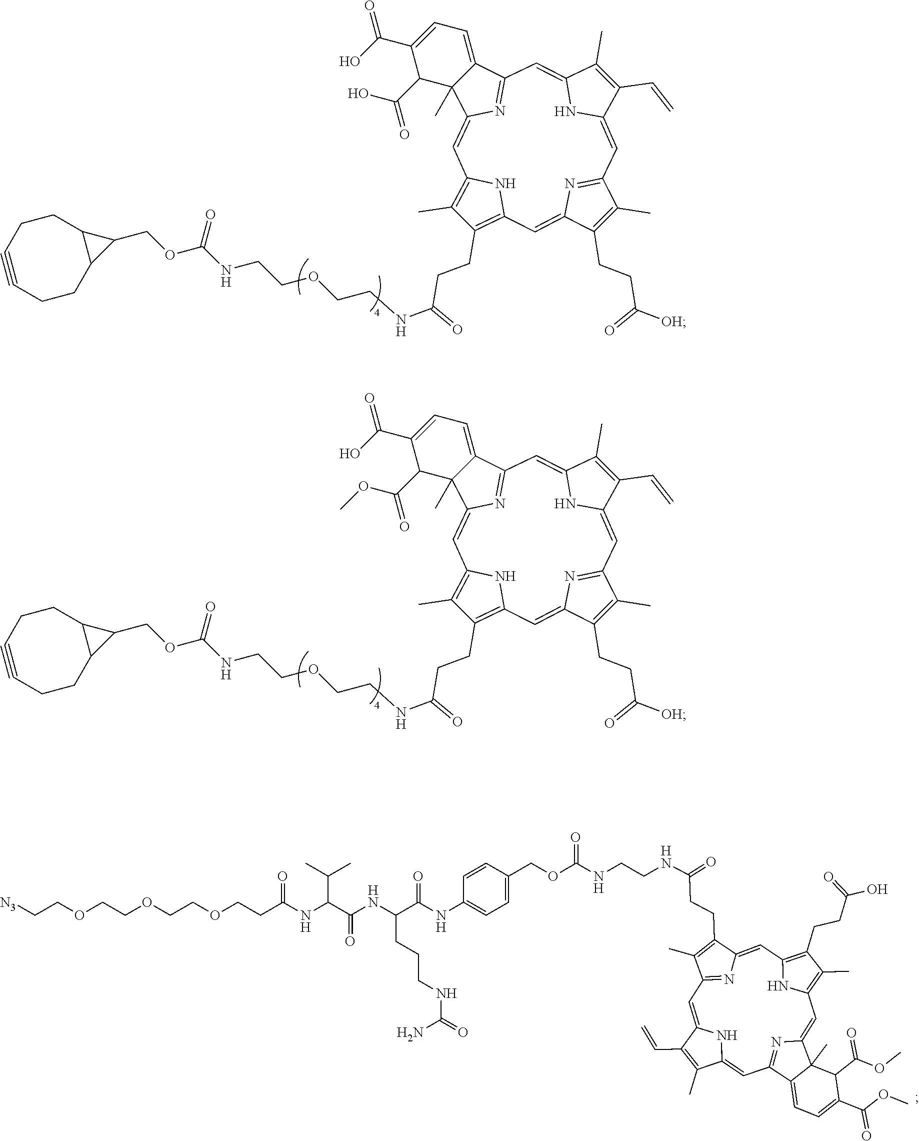 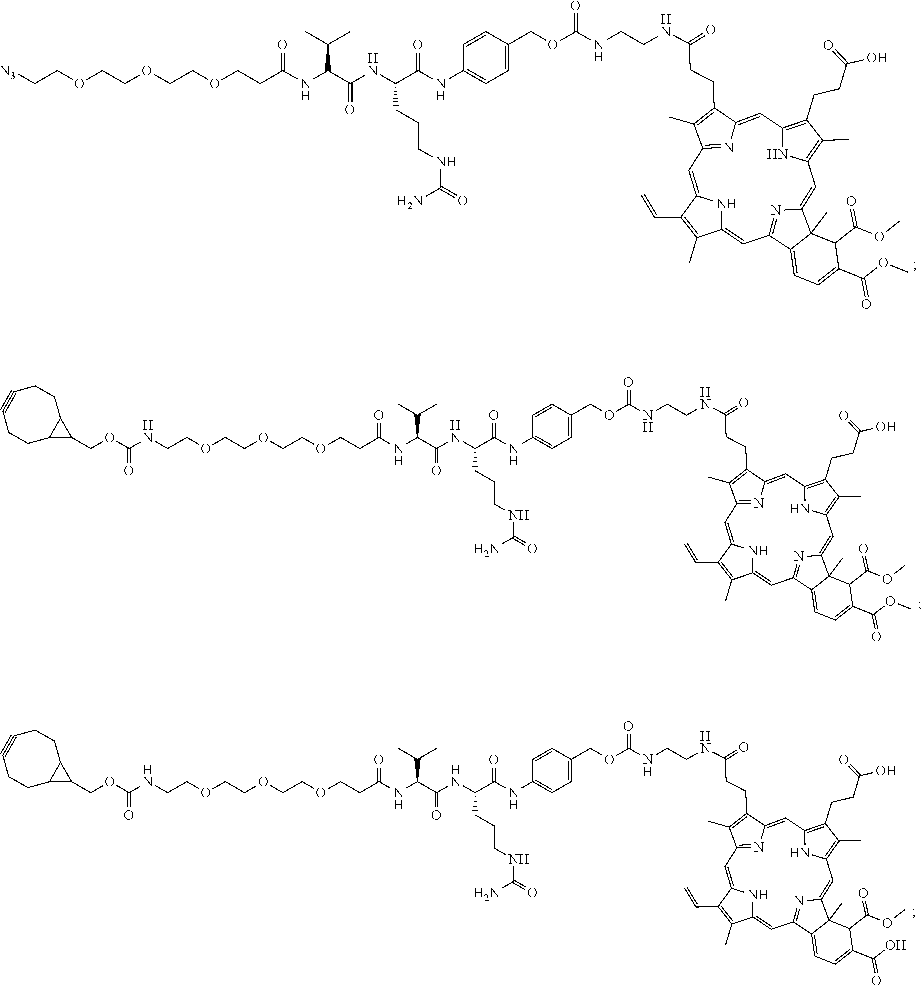 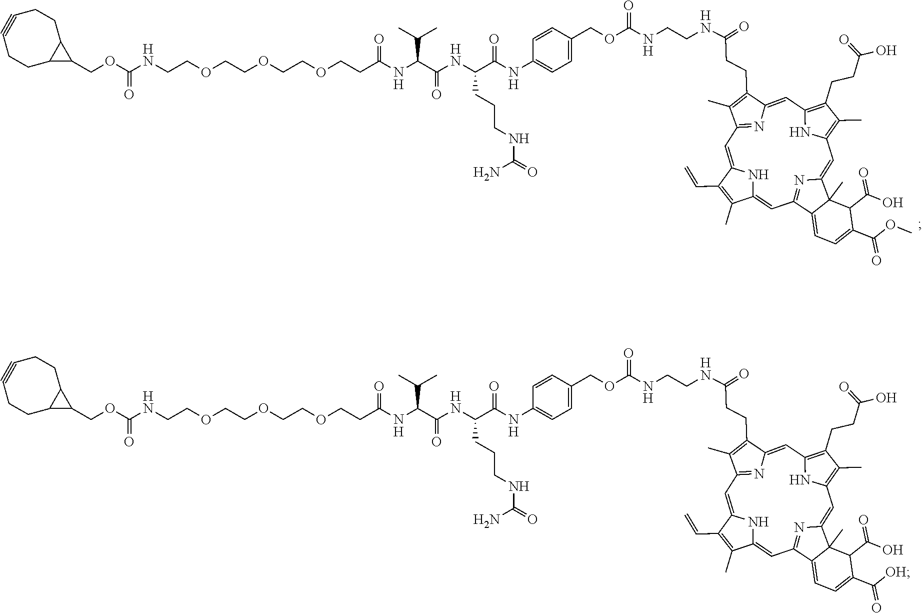 [SMILES: C=Cc1c(C)c2cc3nc(cc4[nH]c(cc5nc(cc1[nH]2)C(C)=C5CCC(=O)NCCNC(=O)OCc1ccc(NC(=O)[C@H](CCCNC(N)=O)NC(=O)[C@@H](NC(=O)CCOCCOCCOCCNC(=O)OCC2C5CCC#CCCC52)C(C)C)cc1)c(CCC(=O)O)c4C)C1(C)C3=CC=C(C(=O)OC)C1C(=O)O ; C=Cc1c(C)c2cc3nc(cc4[nH]c(cc5nc(cc1[nH]2)C(C)=C5CCC(=O)NCCNC(=O)OCc1ccc(NC(=O)[C@H](CCCNC(N)=O)NC(=O)[C@@H](NC(=O)CCOCCOCCOCCNC(=O)OCC2C5CCC#CCCC52)C(C)C)cc1)c(CCC(=O)O)c4C)C1(C)C3=CC=C(C(=O)O)C1C(=O)O] 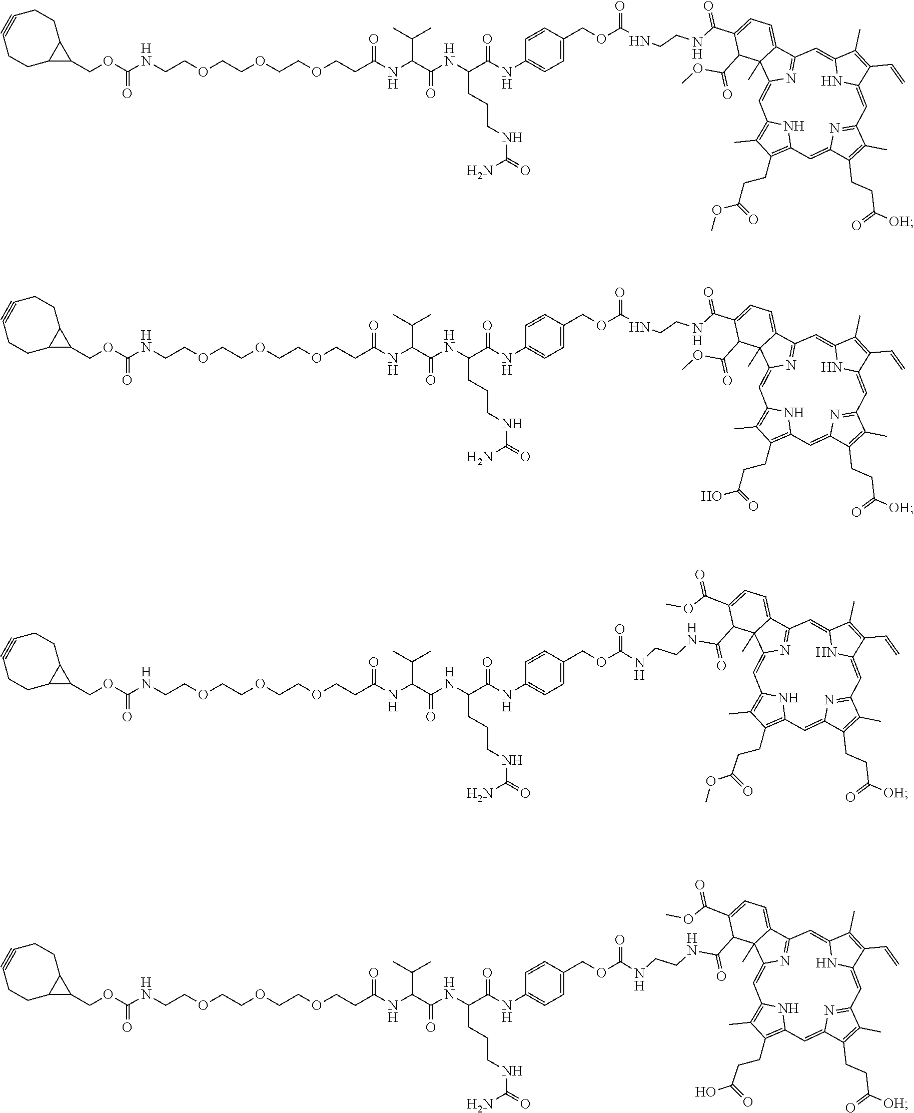 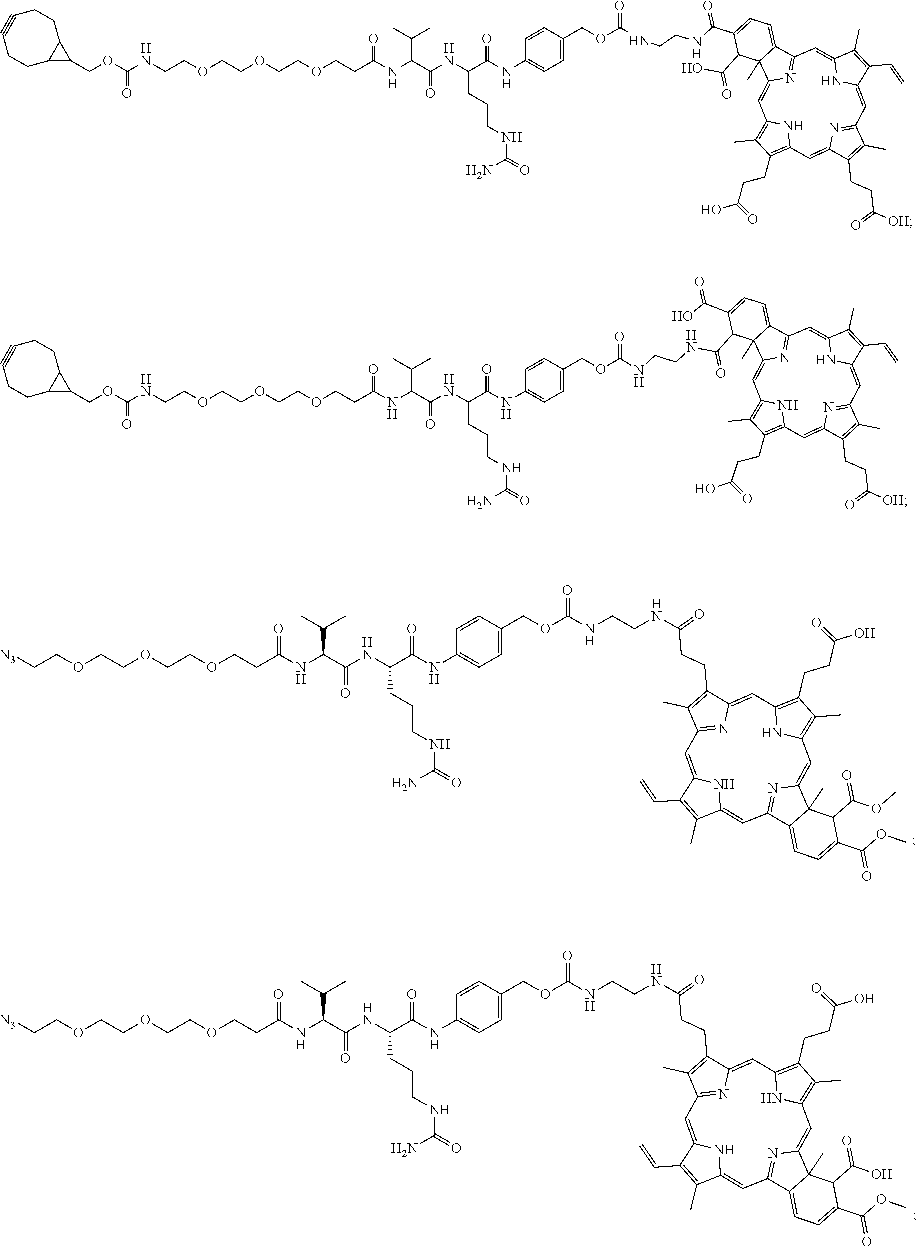 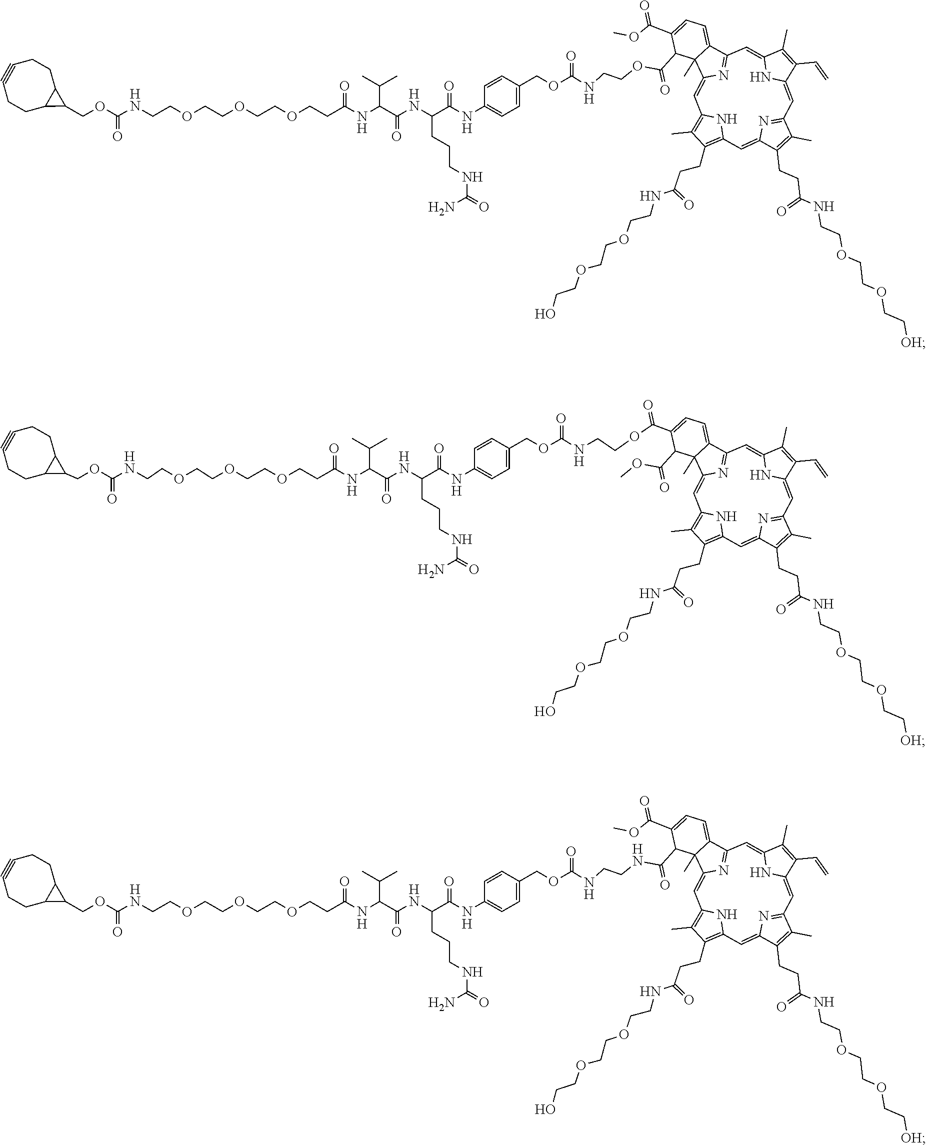 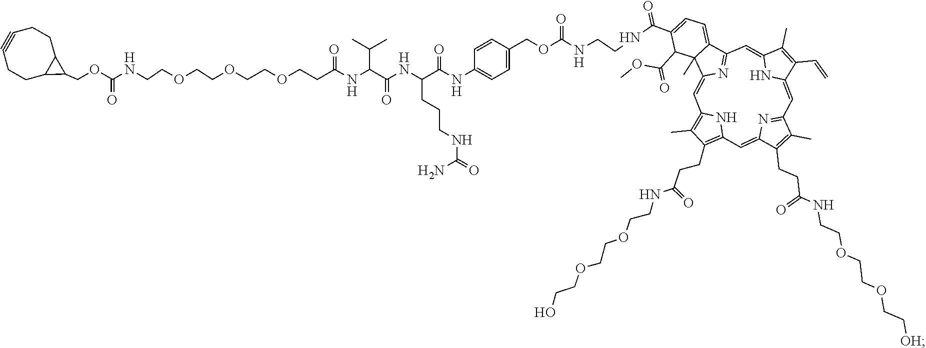 [SMILES: C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)NCCNC(=O)OCc2ccc(NC(=O)C(CCCNC(N)=O)NC(=O)C(NC(=O)CCOCCOCCOCCNC(=O)OCC3C4CCC#CCCC43)C(C)C)cc2)C1C(=O)OC] 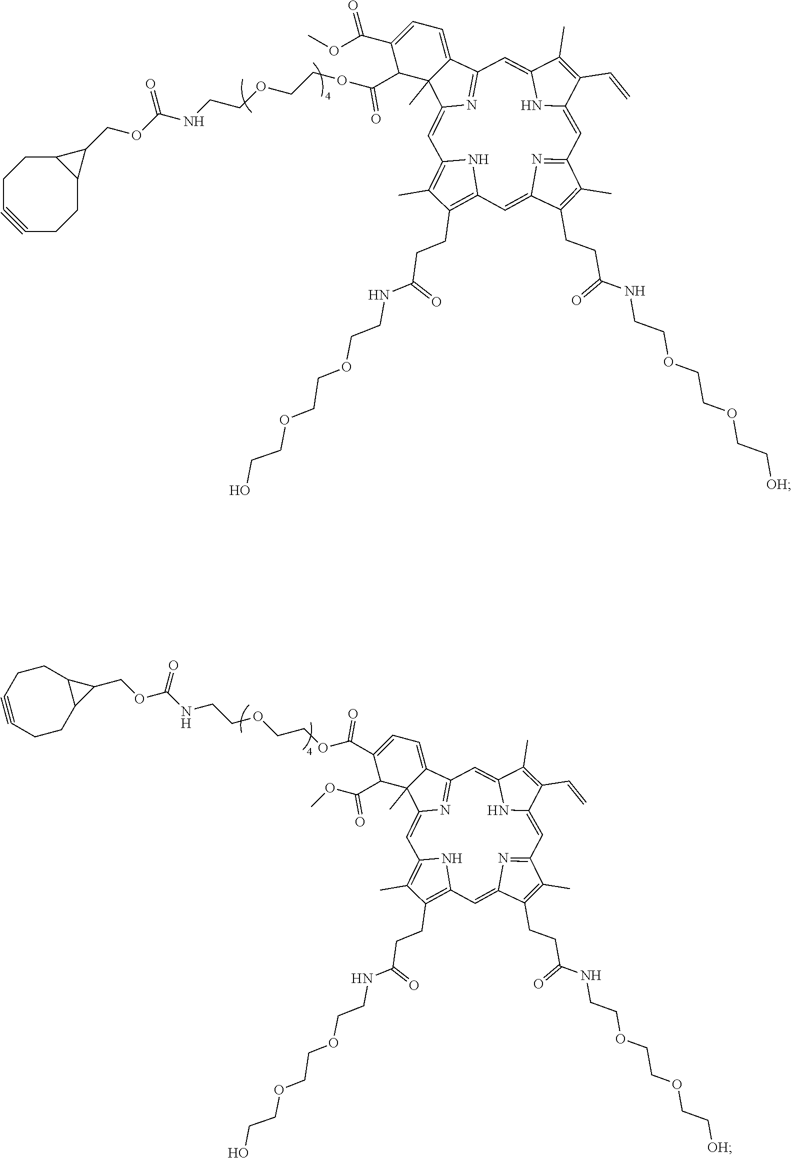 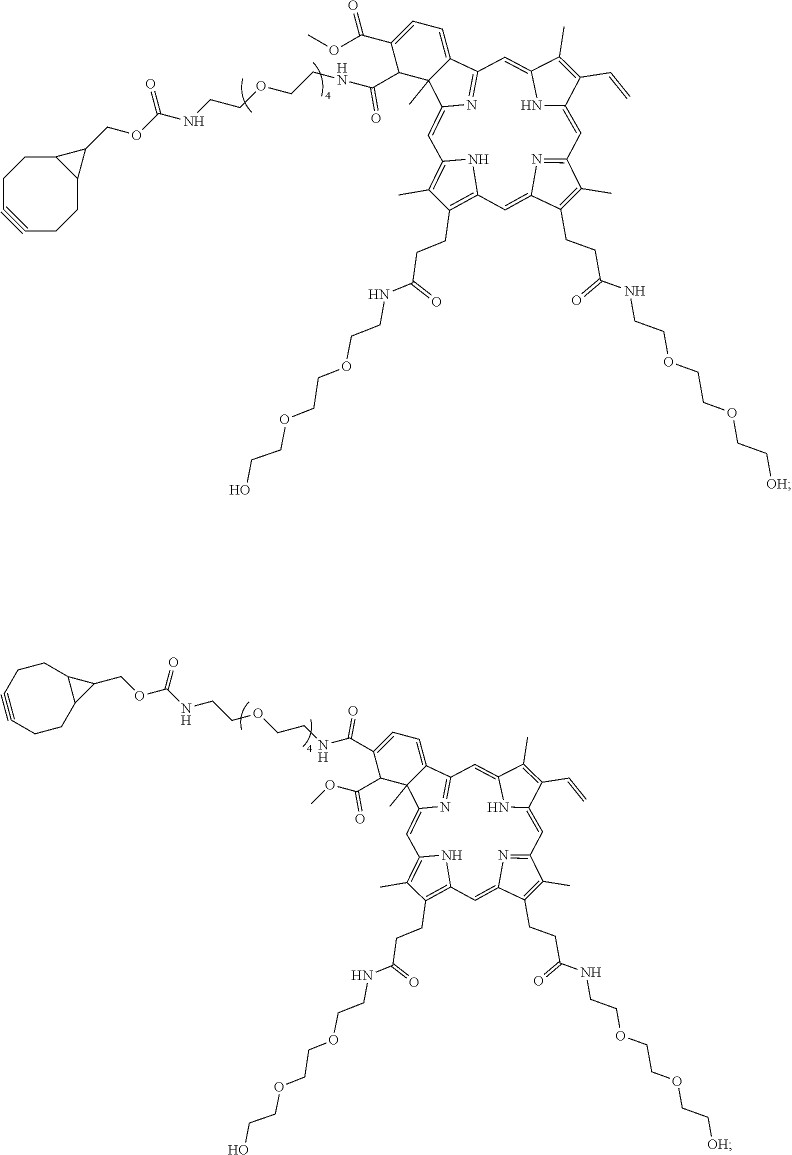 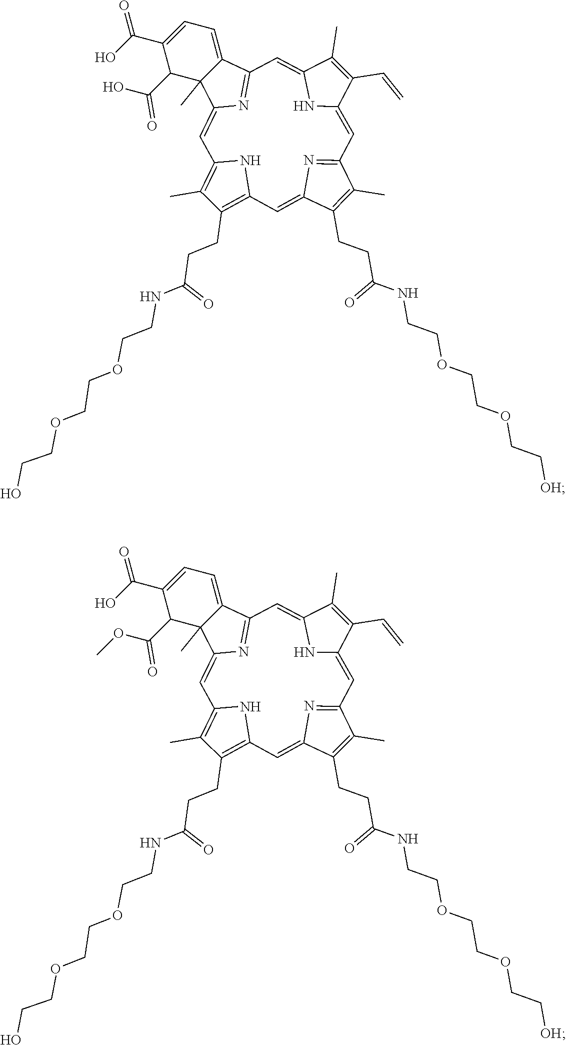 [SMILES: C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)O)C1C(=O)O ; C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)O)C1C(=O)OC] 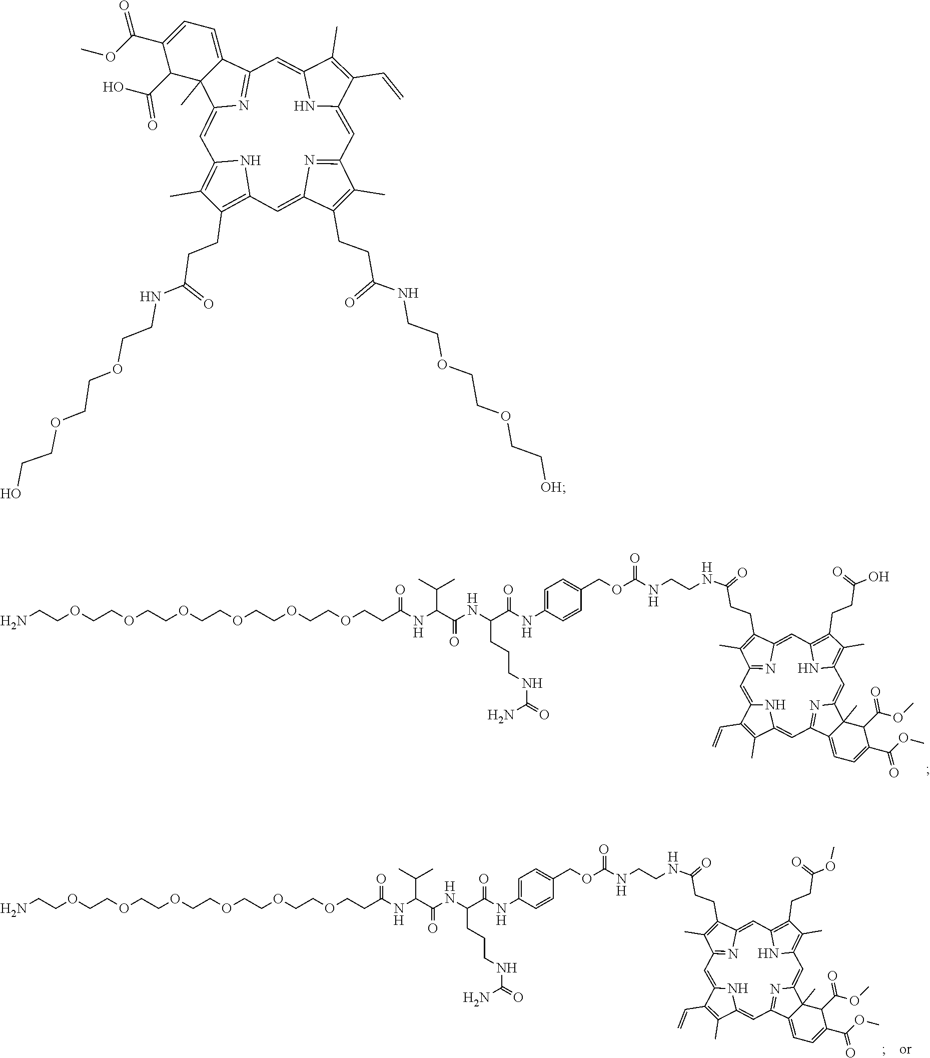 [SMILES: C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)OC)C1C(=O)O ; C=Cc1c(C)c2cc3nc(cc4[nH]c(cc5nc(cc1[nH]2)C(C)=C5CCC(=O)NCCNC(=O)OCc1ccc(NC(=O)C(CCCNC(N)=O)NC(=O)C(NC(=O)CCOCCOCCOCCOCCOCCOCCN)C(C)C)cc1)c(CCC(=O)O)c4C)C1(C)C3=CC=C(C(=O)OC)C1C(=O)OC ; C=Cc1c(C)c2cc3nc(cc4[nH]c(cc5nc(cc1[nH]2)C(C)=C5CCC(=O)NCCNC(=O)OCc1ccc(NC(=O)C(CCCNC(N)=O)NC(=O)C(NC(=O)CCOCCOCCOCCOCCOCCOCCN)C(C)C)cc1)c(CCC(=O)OC)c4C)C1(C)C3=CC=C(C(=O)OC)C1C(=O)OC] 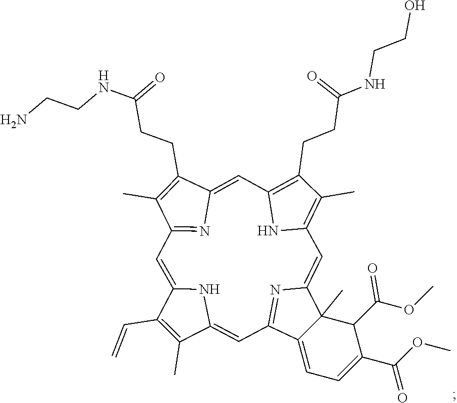 [SMILES: C=Cc1c(C)c2cc3nc(cc4[nH]c(cc5nc(cc1[nH]2)C(C)=C5CCC(=O)NCCN)c(CCC(=O)NCCO)c4C)C1(C)C3=CC=C(C(=O)OC)C1C(=O)OC]
or a pharmaceutically acceptable salt thereof, a regioisomer, or a pharmaceutically acceptable salt of the regioisomer.

8. A linker-photosensitizer compound of formula (II): 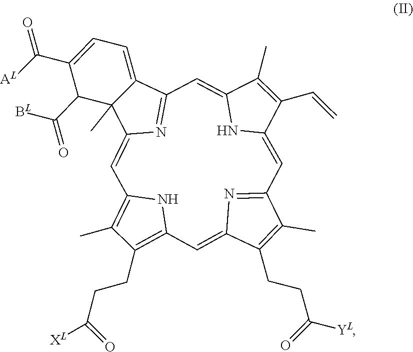
or a pharmaceutically acceptable salt thereof, wherein:
A^{L}, B^{L}, X^{L} and Y^{L} are each independently —OH, —OC₁₋₄alkyl, —N(R¹⁰⁰)(R¹⁰¹) or —Z-L^{P}-linker-L^{A}-R^{A}, provided one of A^{L}, B^{L}, X^{L} and Y^{L} is —Z-L^{P}-linker-L^{A}-R^{A};
R¹⁰⁰ is H or C₁₋₃alkyl;
R¹⁰¹ is H, C₁₋₃alkyl or —(CH₂—CH₂—O)ₙᵣR¹⁰²;
R¹⁰² is H or Me;
nr is an integer from 1 to 16;
Z is —O— or —NR¹—;
R¹ is H or C₁₋₃alkyl;
L^{P} and L^{A} are each independently a spacer;
Linker is connection moiety formed by a first reactive group attached to L^{P} and a second reactive group attached to L^{A}; and
R^{A} is a reactive group that can form a covalent bond with a targeting agent.

9. The compound of claim 8, or a pharmaceutically acceptable salt thereof, wherein R^{A} is a reactive ester, —NH₂, or a maleimide group, optionally, wherein R^{A} is 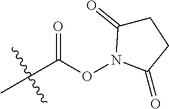 [SMILES: *C(=O)ON1C(=O)CCC1=O]
or —NH₂.

10. The compound of claim 8 or 9, or a pharmaceutically acceptable salt thereof, wherein:
(i) L^{A} is a spacer comprising a hydrophilic moiety;
(ii) L^{A} is a spacer comprising a PEG moiety; or
(iii) L^{A} is *—CH₂CH₂(OCH₂CH₂)ₚ—, wherein * is the site connected to R^{A}; and p is an integer from 0 to 30, optionally, wherein p is an integer from 2 to 16, from 2 to 10, from 2 to 8, or from 3 to 5; or p is 2, 3, 4, 5, 6, 7 or 8.

11. The compound of any one of claims 8-10, wherein:
(i) the first reactive group and the second reactive group are each selected from a maleimide group, a thiol group, a cyclooctyne group, and an azido group; or
(ii) Linker is represented by the following formula: 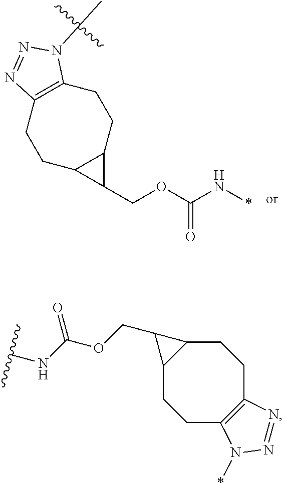 [SMILES: *NC(=O)OCC1C2CCc3nnn(*)c3CCC21 ; *NC(=O)OCC1C2CCc3nnn(*)c3CCC21]
wherein *- represents a bond to L^{P} and 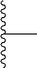
represents a bond to L^{A}.

12. The compound of any one of claims 8-11, or a pharmaceutically acceptable salt thereof, wherein:
a) A^{L} and B^{L} are —OCH₃; and one of X^{L} and Y^{L} is —OCH₃ and the other one of X^{L} and Y^{L} is —Z-L^{P}-linker-L^{A}-R^{A};
b) A^{L} and B^{L} are —OCH₃; and one of X^{L} and Y^{L} is —OH and the other one of X^{L} and Y^{L} is —Z-L^{P}-linker-L^{A}-R^{A};
c) one of A^{L} and B^{L} is —OCH₃, and the other one of A^{L} and B^{L} is —OH; and one of X^{L} and Y^{L} is —OH and the other one of X^{L} and Y^{L} is —Z-L^{P}-linker-L^{A}-R^{A};
d) A^{L} and B^{L} are —OH; and one of X^{L} and Y^{L} is —OH and the other one of X^{L} and Y^{L} is —Z-L^{P}-linker-L^{A}-R^{A}; or
e) one of A^{L} and B^{L} is —Z-L^{P}-linker-L^{A}-R^{A} and the other one of A^{L} and B^{L} is —OH or —OCH₃; and X^{L} and Y^{L} are both —NH(CH₂—CH₂—O)ₙᵣR¹⁰², wherein R¹⁰² is H or Me; and nr is an integer from 2 to 8.

13. The compound of claim 12, or a pharmaceutically acceptable salt thereof, wherein:
(i) the compound is represented by the following formula: 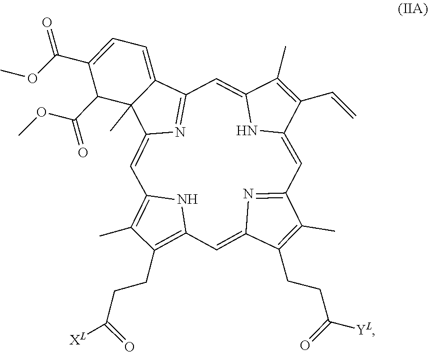
or a pharmaceutically acceptable salt thereof, wherein one of X^{L} and Y^{L} is —Z-L^{P}-linker-L^{A}-R^{A} and the other is —OH; or
(ii) the compound is represented by the following formula: 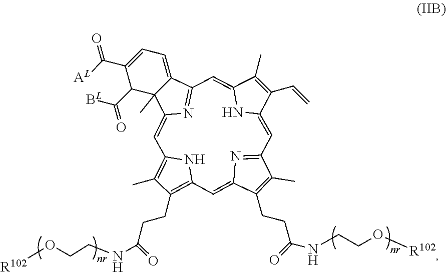
or a pharmaceutically acceptable salt thereof, wherein one of A^{L} and B^{L} is —Z-L^{P}-linker-L^{A}-R^{A} and the other is —OH or —OCH₃; R¹⁰² is H or Me; and nr is an integer from 2 to 8.

14. The compound of any one of claims 8-13, or a pharmaceutically acceptable salt thereof, wherein:
(i) Z is —NH—; or
(ii) Z is —O—.

15. The compound of any one of claims 8-14, or a pharmaceutically acceptable salt thereof, wherein:
(i) L^{P} is a non-cleavable spacer, optionally, wherein:
(a) L^{P} comprises a hydrophilic moiety;
(b) L^{P} comprises a PEG moiety;
(c) L^{P} is *—CH₂CH₂—(OCH₂CH₂)ₘ—, wherein * is the site connected to ker, and m is an integer from 0 to 30, optionally, wherein m is an integer from 2 to 16, from 2 to 10, from 2 to 8, or from 3 to 5; or m is 2, 3, 4, 5, 6, 7 or 8; or
(d) L^{P} is *—CH₂CH₂—(OCH₂CH₂)₄—;
(ii) L^{P} is a cleavable spacer, optionally, wherein:
(a) the cleavable spacer comprises an enzyme labile group, a pH labile group, a disulfide group, a ROS cleavable group, a photocleavable group, optionally, wherein:
(1) the enzyme labile group is a group cleavable by an esterase, a peptidase, an aminopeptidase, a β-galactosidase, a β-glucuronidase, a carboxylesterase, a caspase, a diaphorase, a histone deacetylase, a legumain, or matrix metalloproteinase; or
(2) the enzyme labile group is a group cleavable by cathepsin B;
(ii) the spacer represented by L^{P} comprises a peptide and a self-immolative group;
(iii) the spacer L^{P} is presented by the following formula: 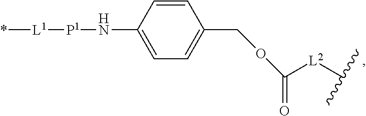
wherein:
*- represents a bond to the reactive group represented by ker; 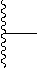
represents a bond to the group Z;
L¹ and L² are each independently a connecting spacer;
P¹ is a peptide comprising 2 to 5 amino acid residues, optionally, wherein:
(a1) P¹ is a peptide selected from Phe-Arg-Arg-Gly, Glu-Val-Cit, Val-Cit, Cit-Val, Gly-Gly-Phe, Val-Ala, and Ala-Val; or
P¹ is: 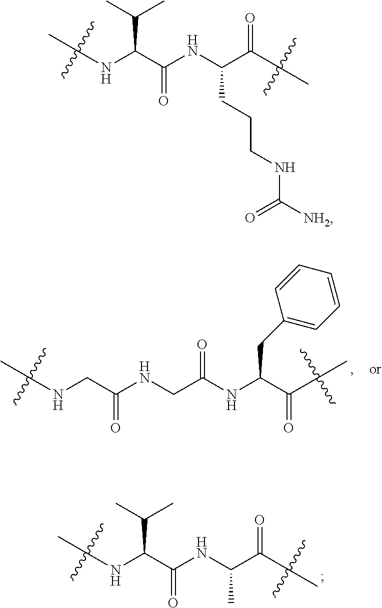 [SMILES: *N[C@H](C(=O)N[C@@H](CCCNC(N)=O)C(*)=O)C(C)C ; *NCC(=O)NCC(=O)N[C@@H](Cc1ccccc1)C(*)=O ; *N[C@H](C(=O)N[C@@H](C)C(*)=O)C(C)C]
(a2) L¹ comprises a hydrophilic moiety;
L¹ comprises a PEG moiety;
L¹ is *—CH₂—CH₂—(OCH₂CH₂)ₙ—C(═O)—**; n is an integer from 0 to 30; *-represents a bond to the reactive group represented by ker; and **- represents a bond to P¹, optionally, wherein n is an integer from 2 to 16, from 2 to 10, from 2 to 8, or from 3 to 5; or n is 2, 3, 4, 5, 6, 7 or 8; or
L¹ is *—CH₂—CH₂—(OCH₂CH₂)₃—C(═O)—**; and/or
(a3) L² is —NH—C₁₋₆alkyl-**, wherein ** is the site connected to Z; or
L² is —NH—CH₂CH₂—**; wherein ** is the site connected to Z.

16. The compound of claim 8, wherein the compound is represented by the following formula: 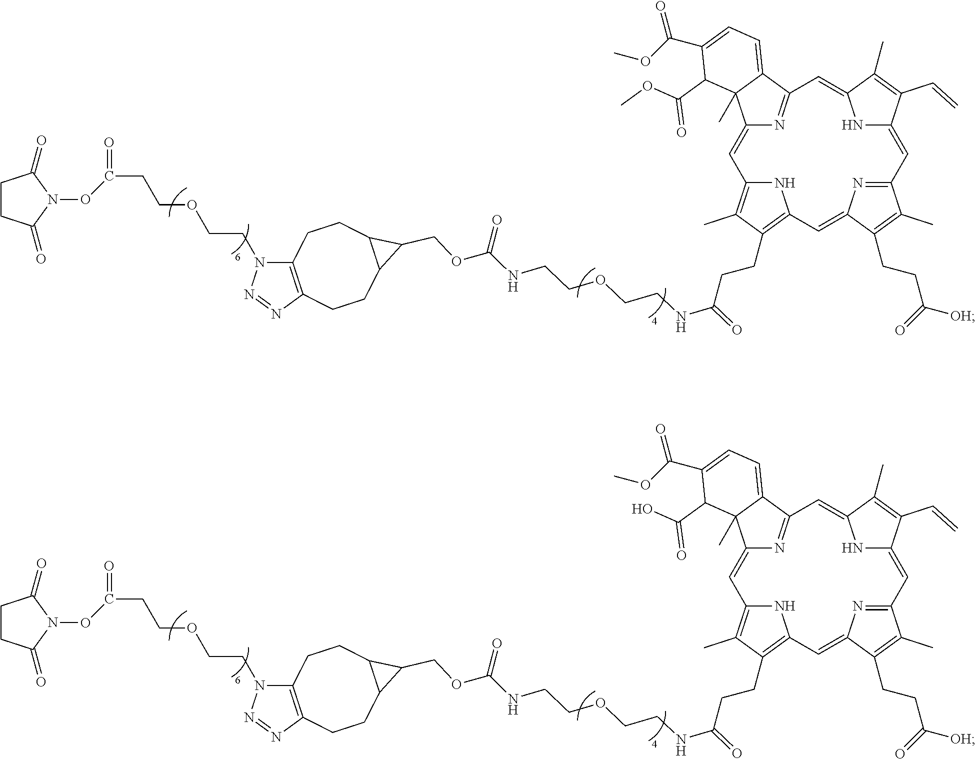 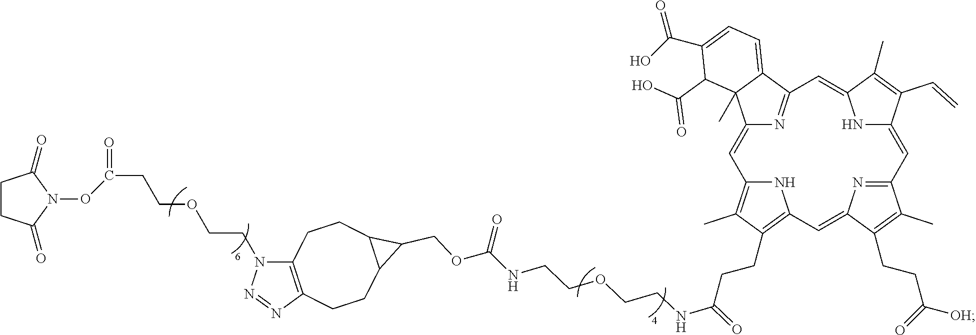 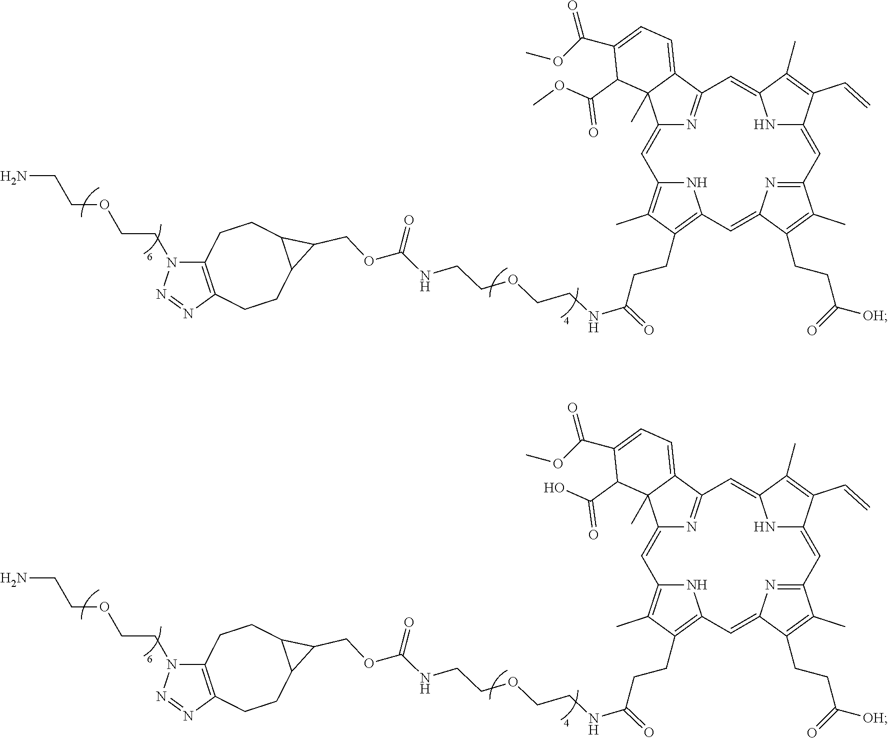 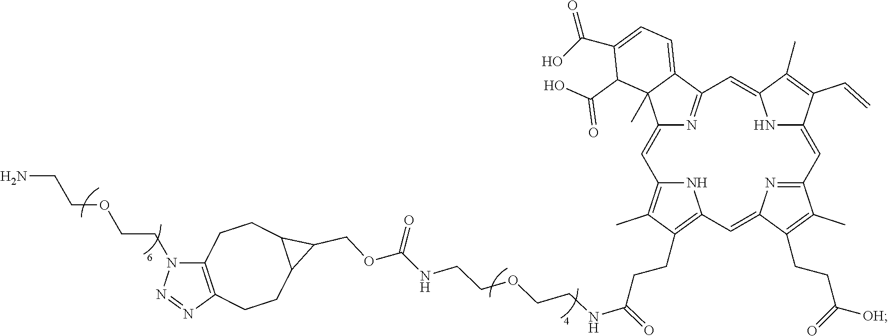 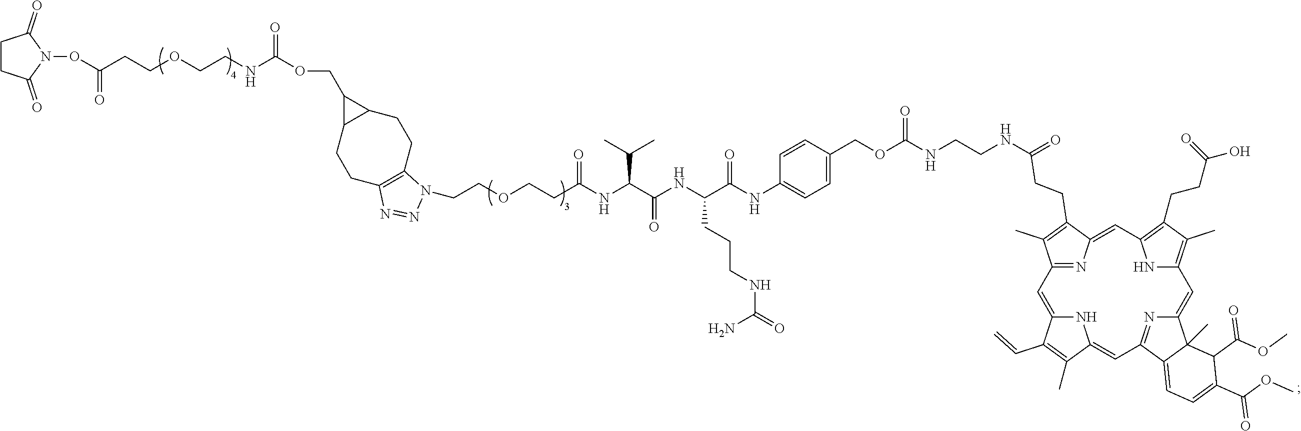 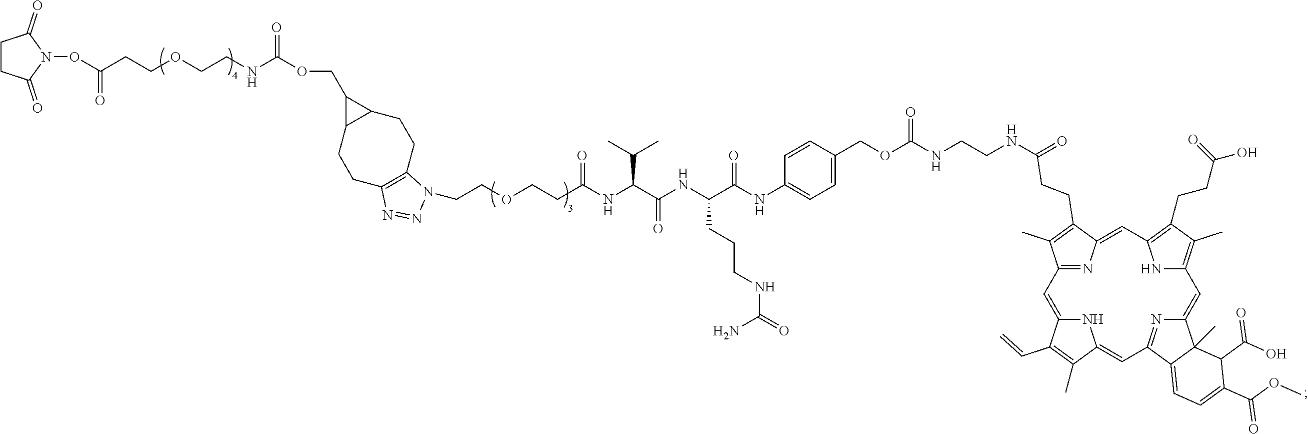 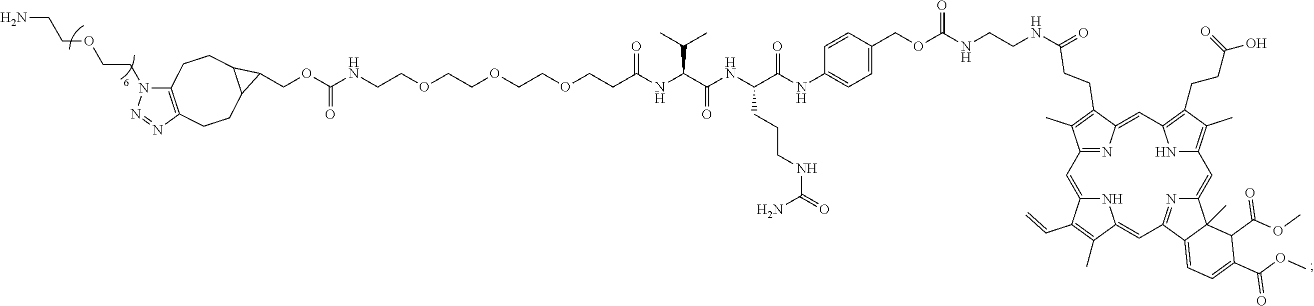 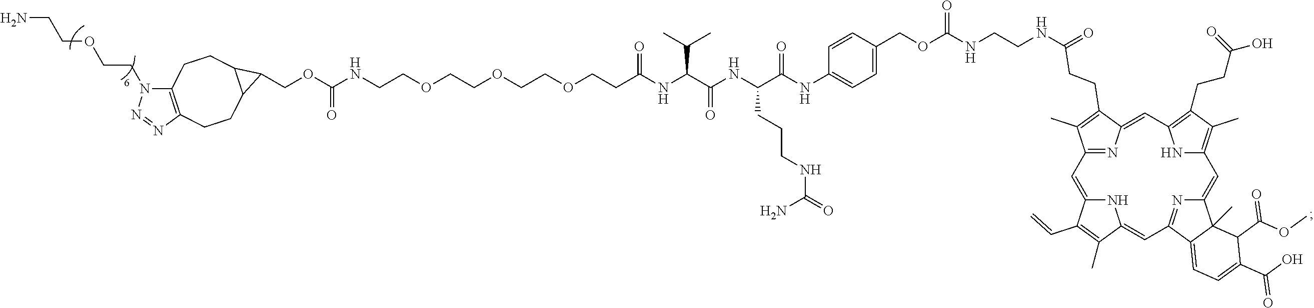 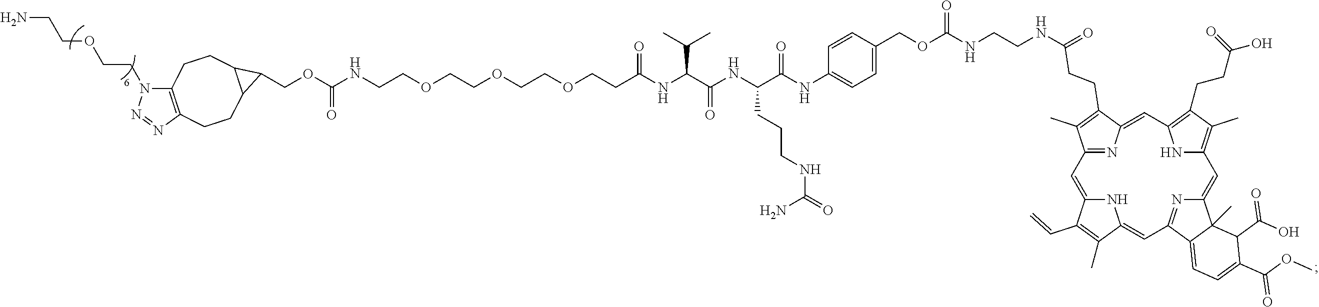 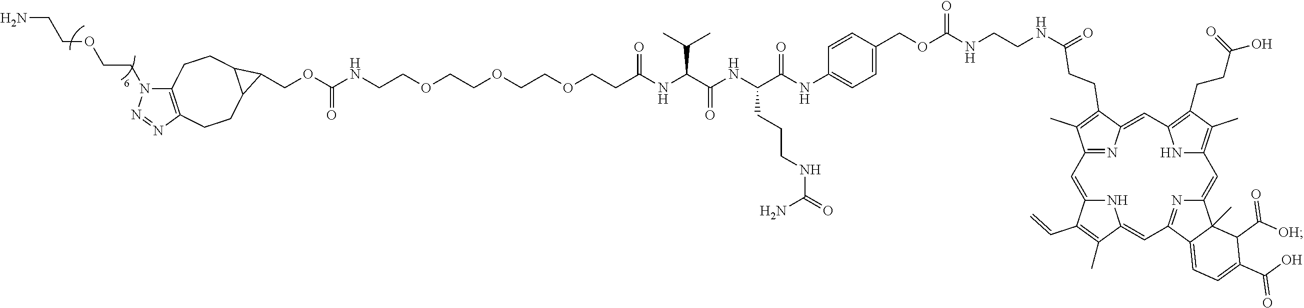 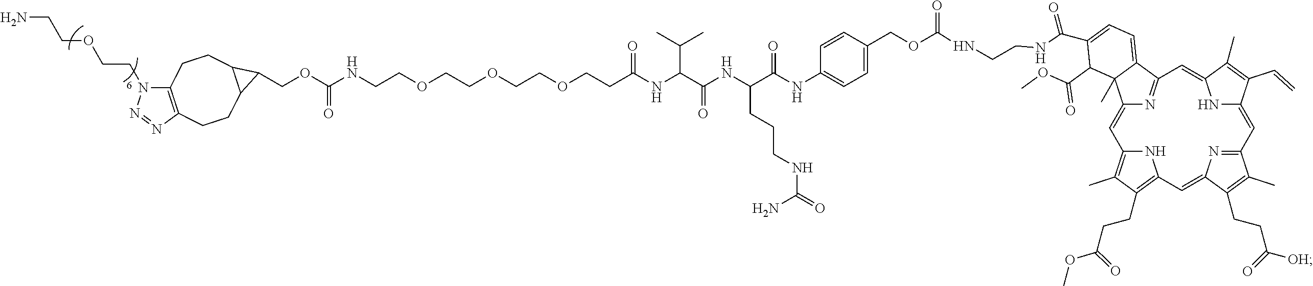 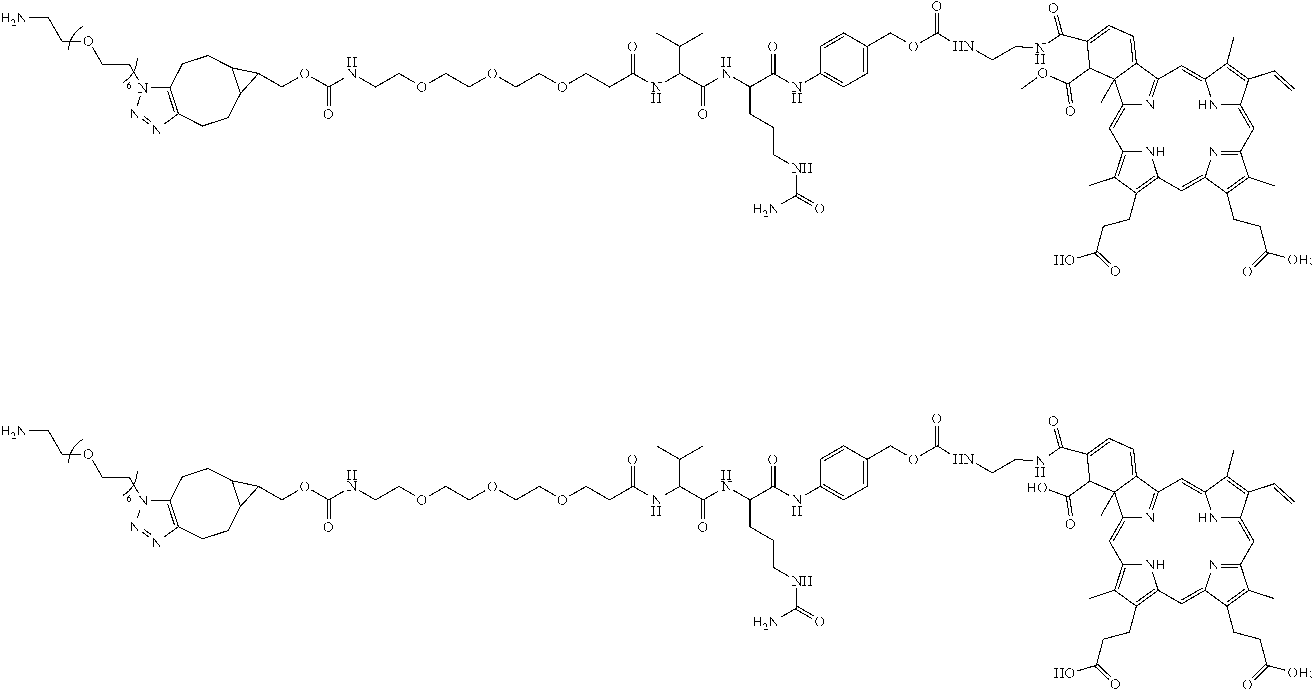 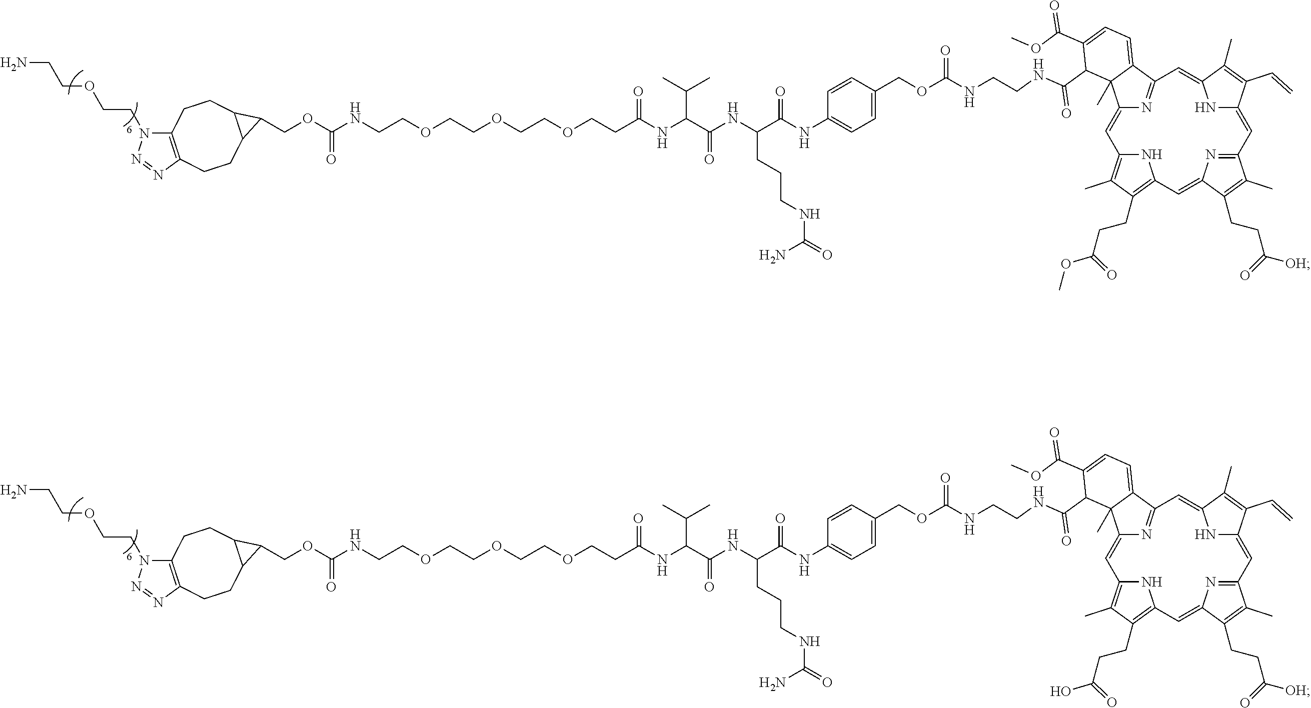 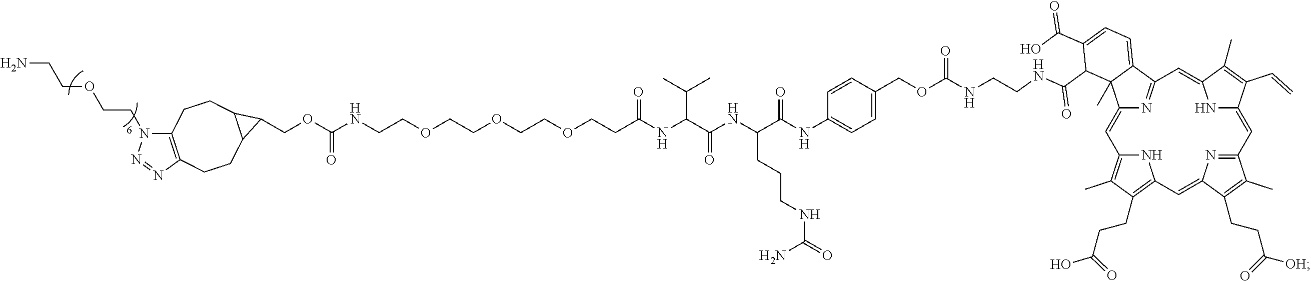 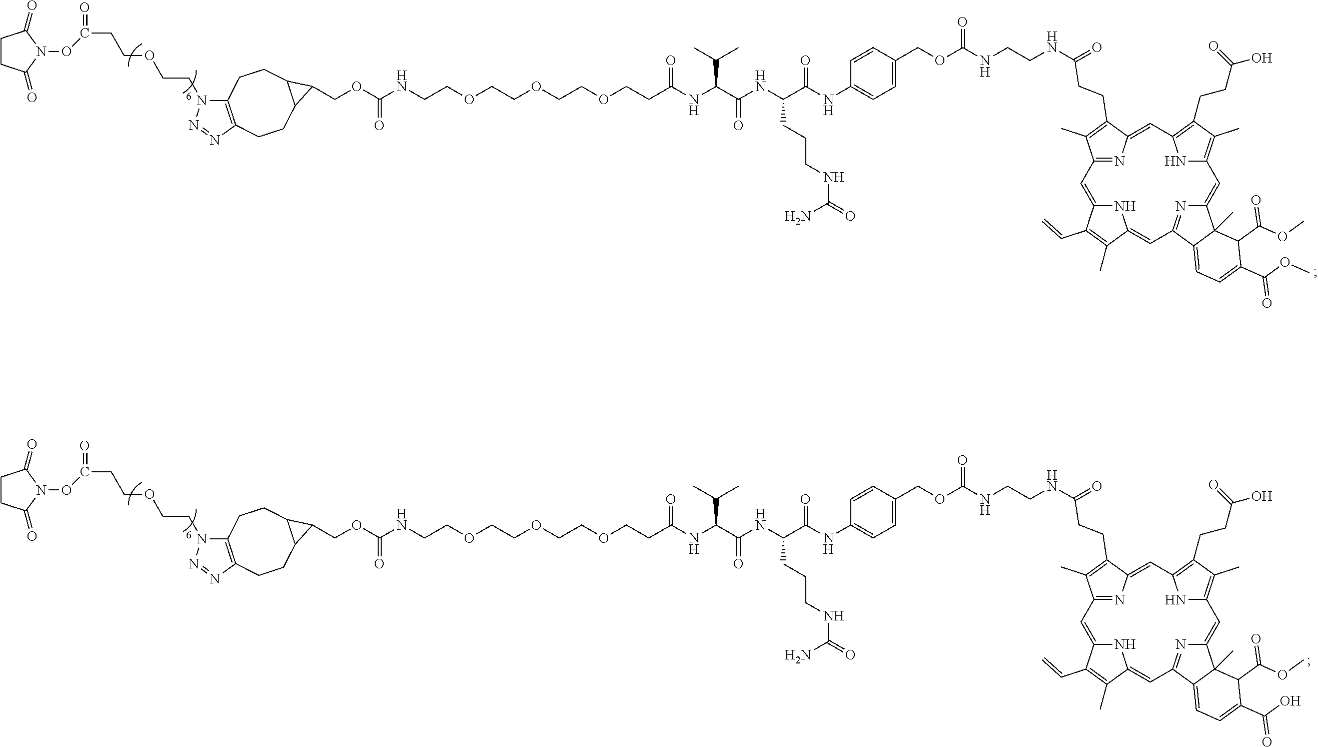 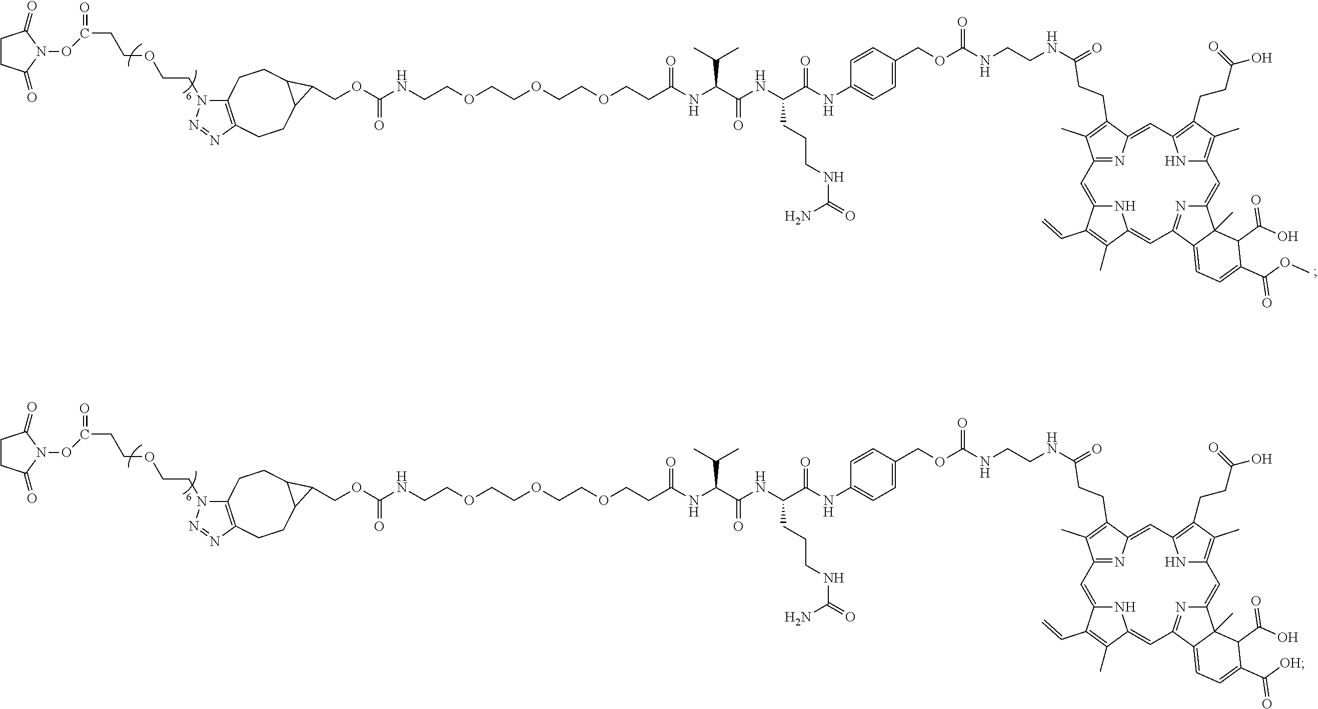 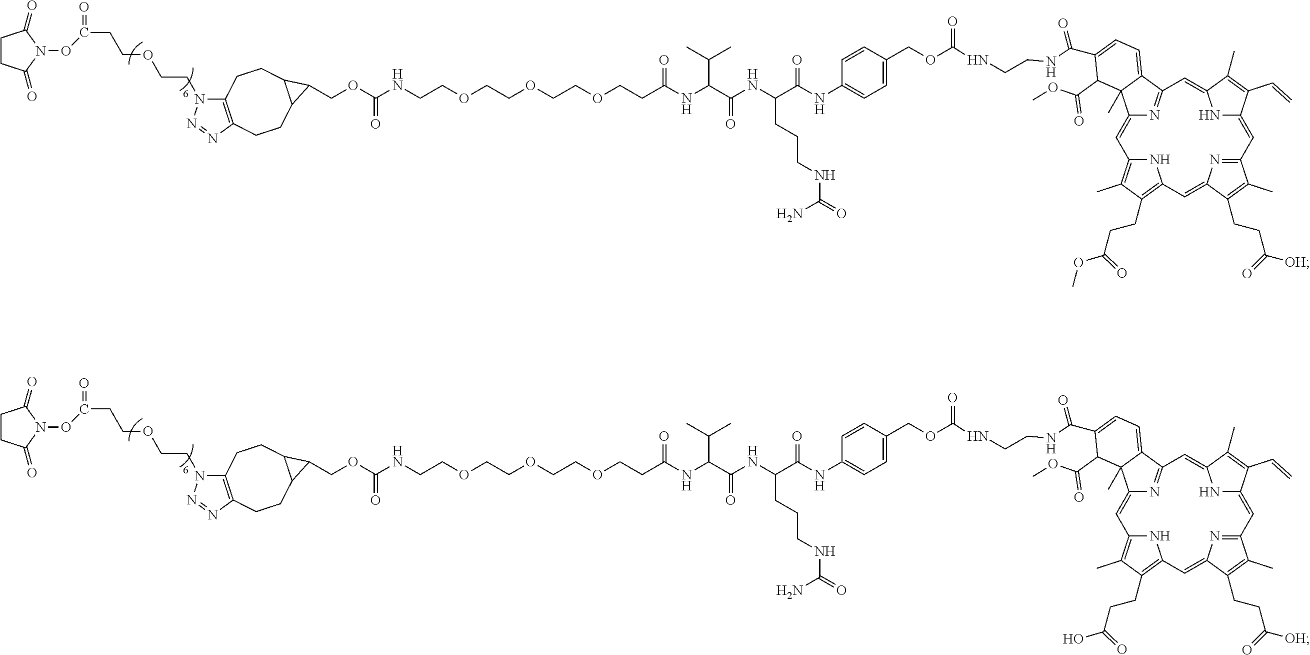 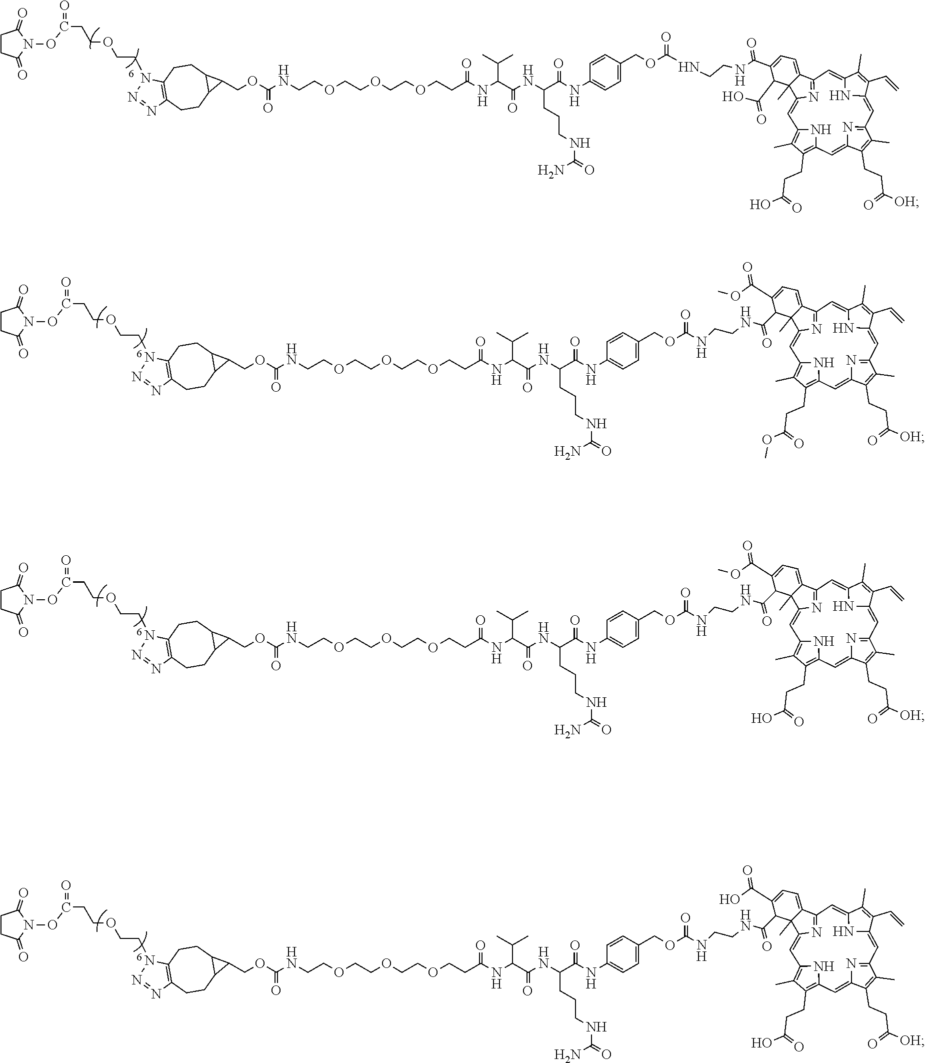 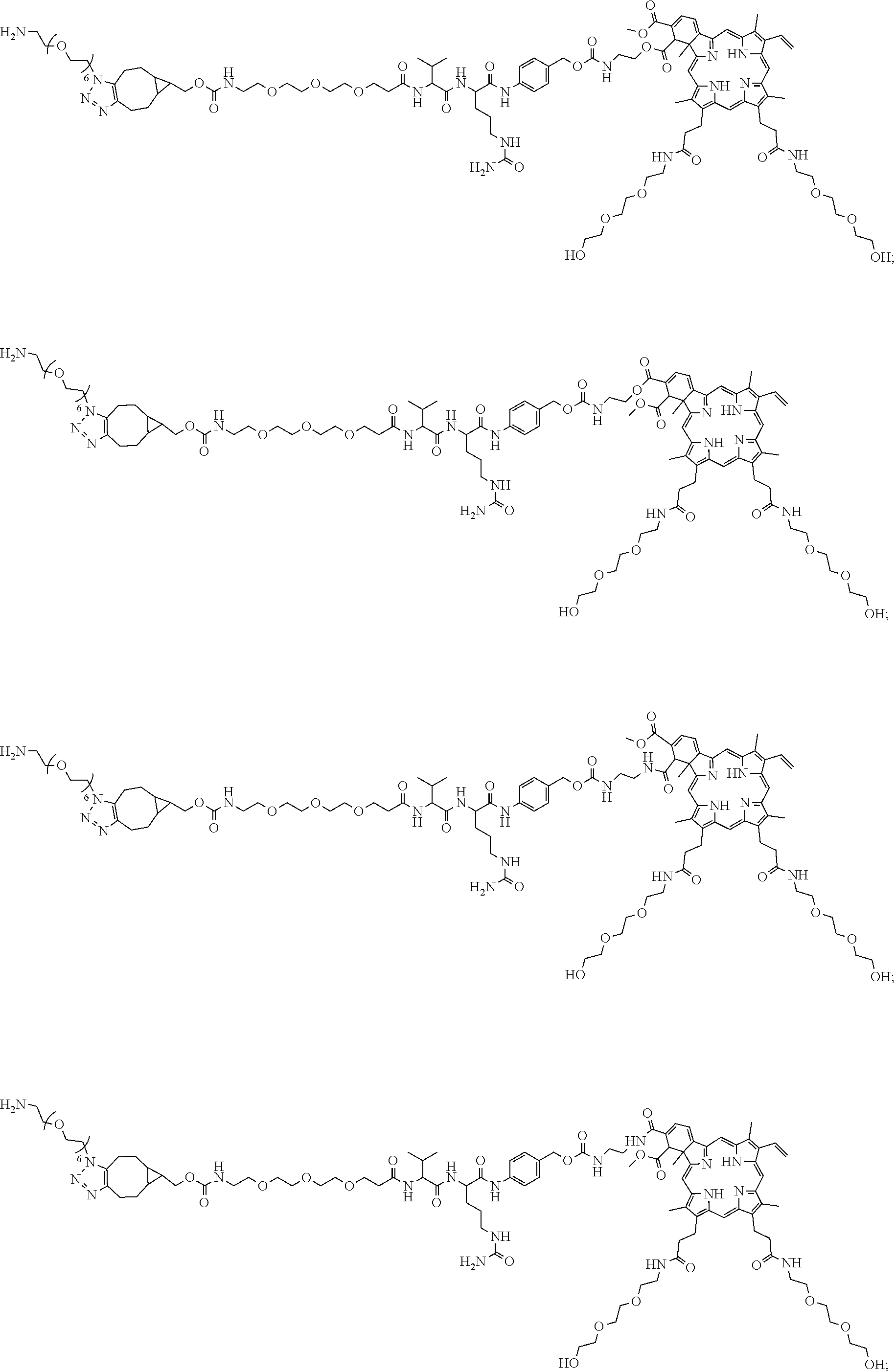 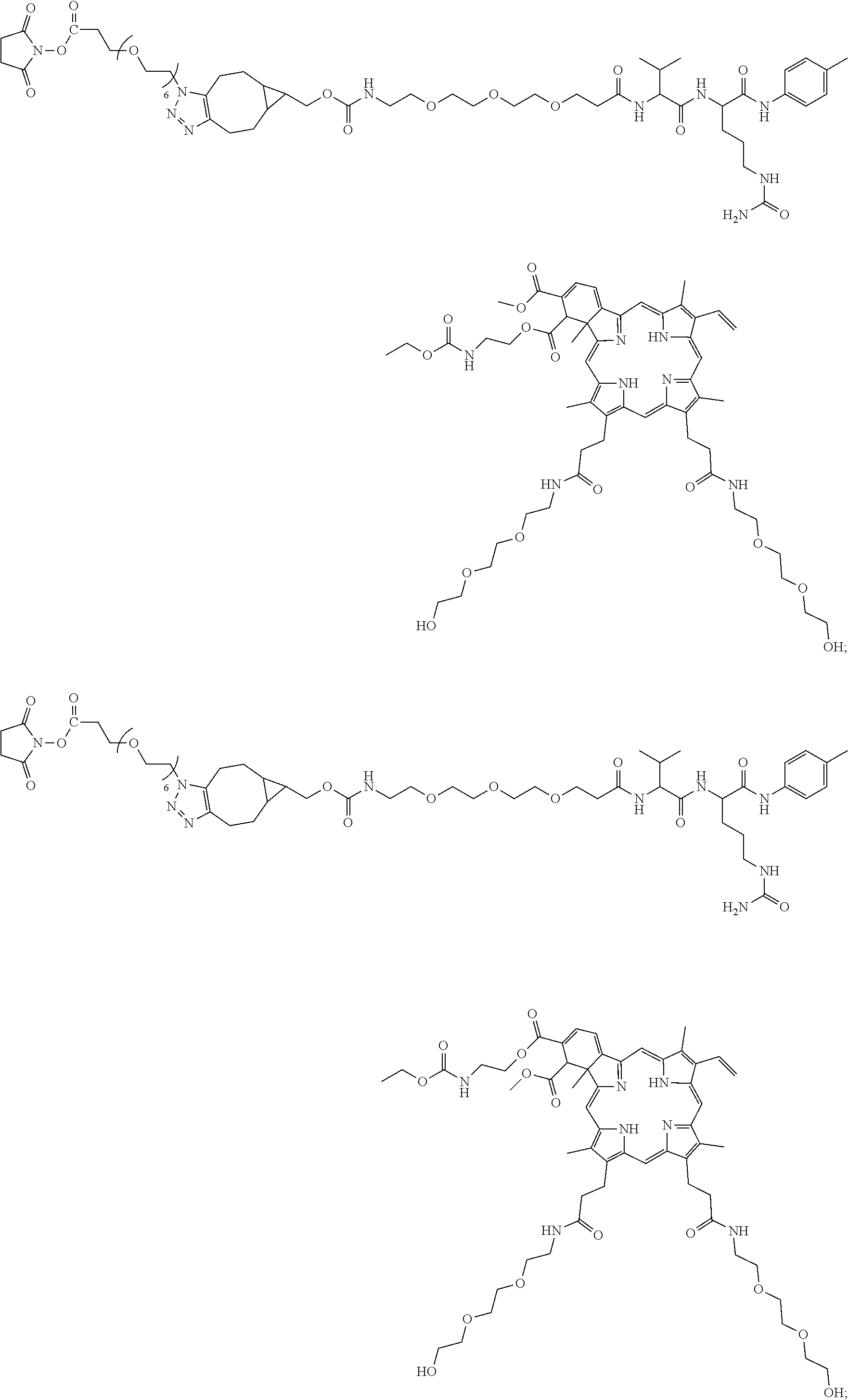 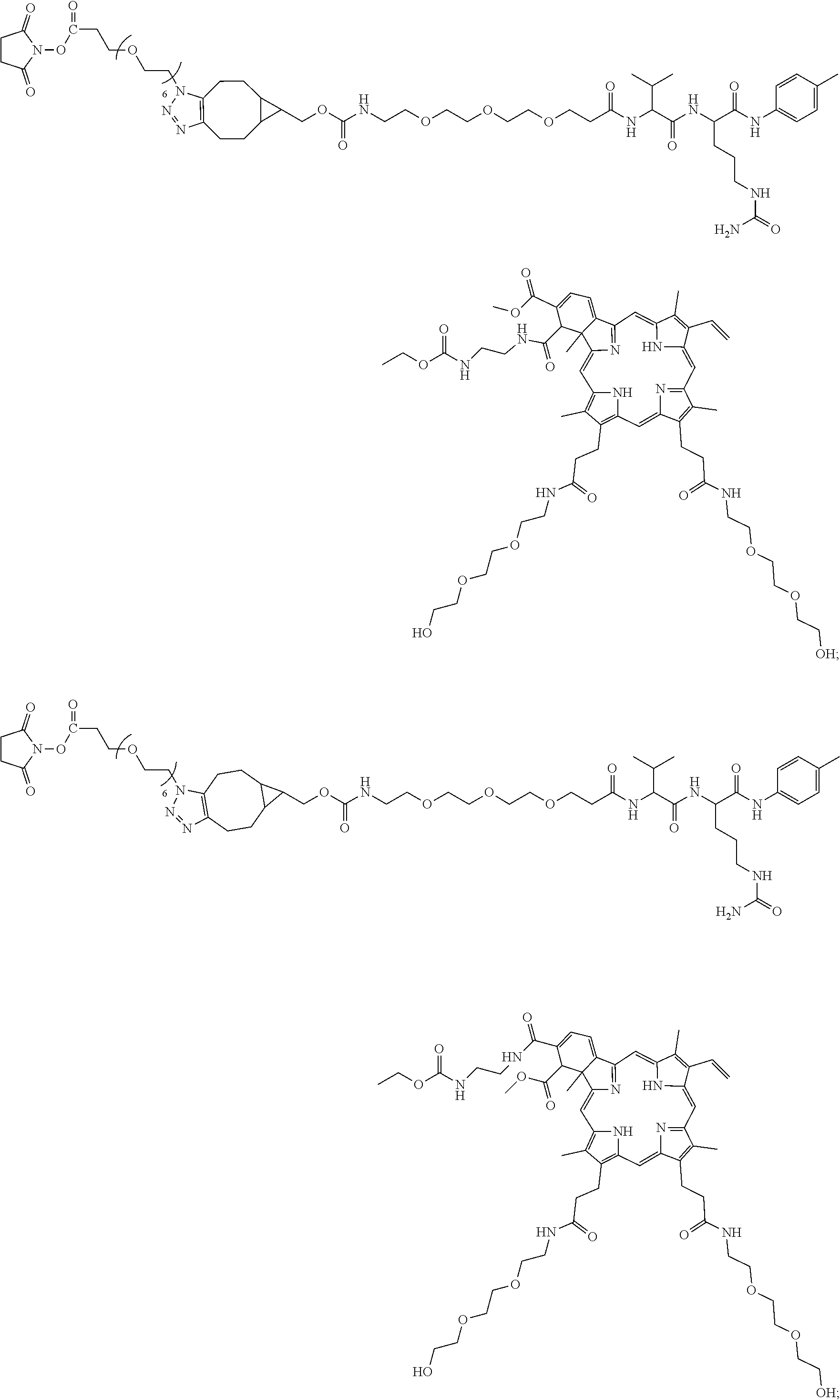 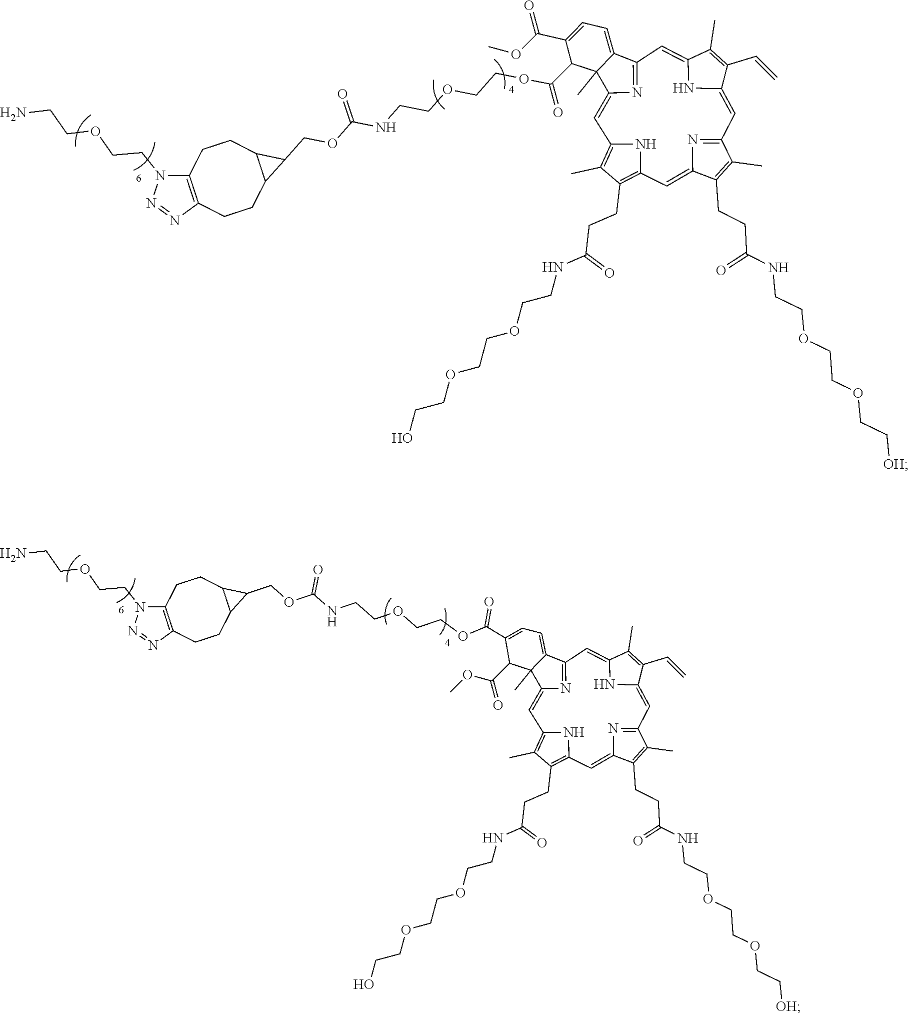 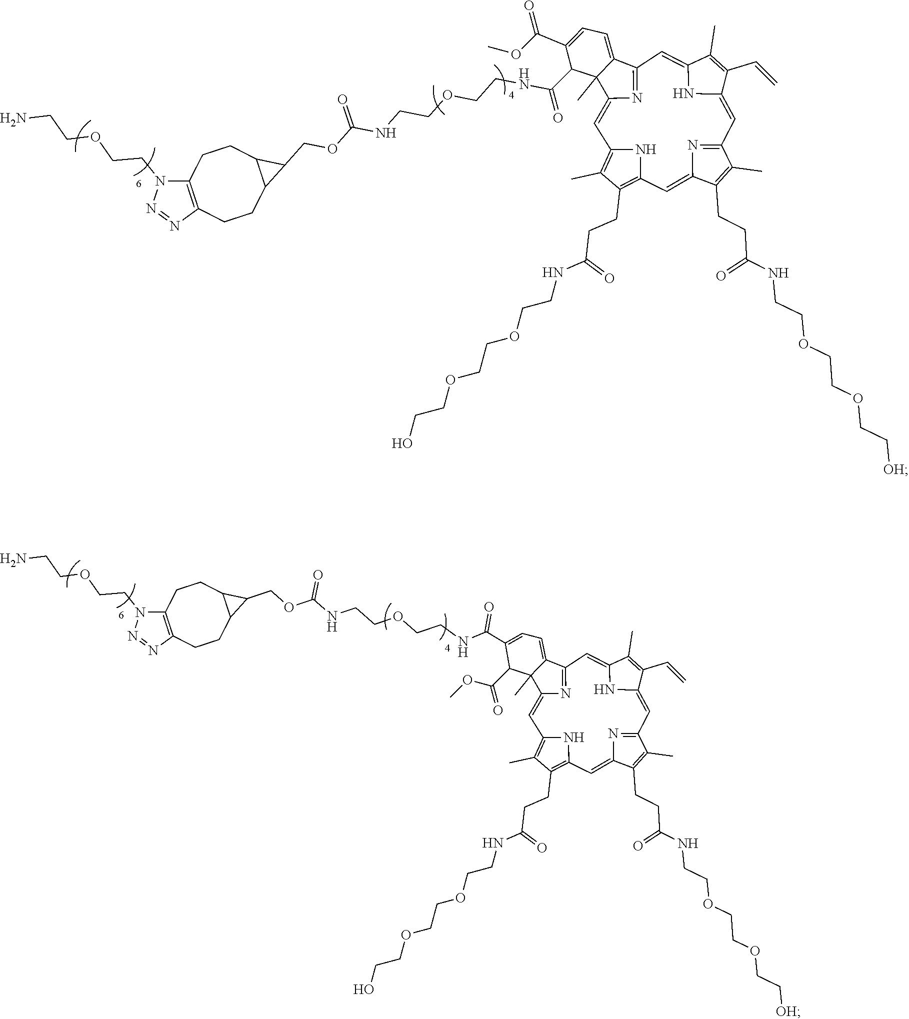 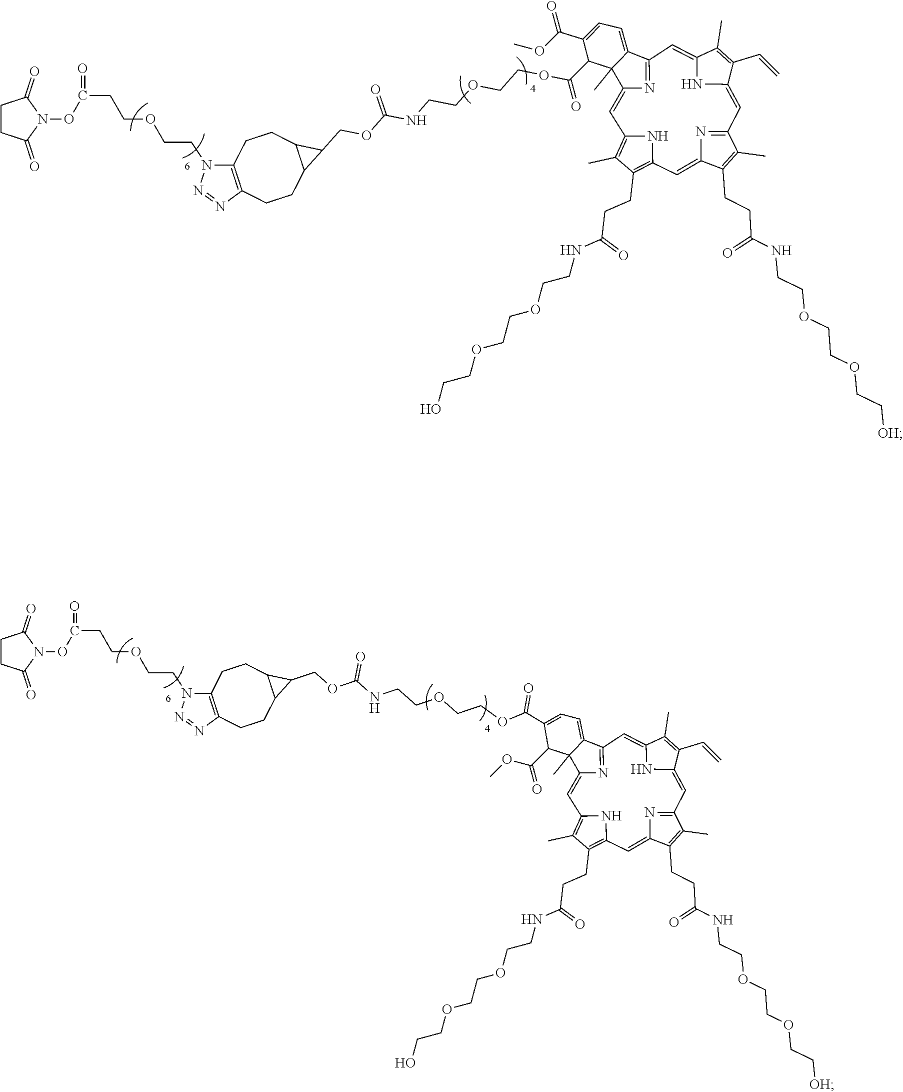 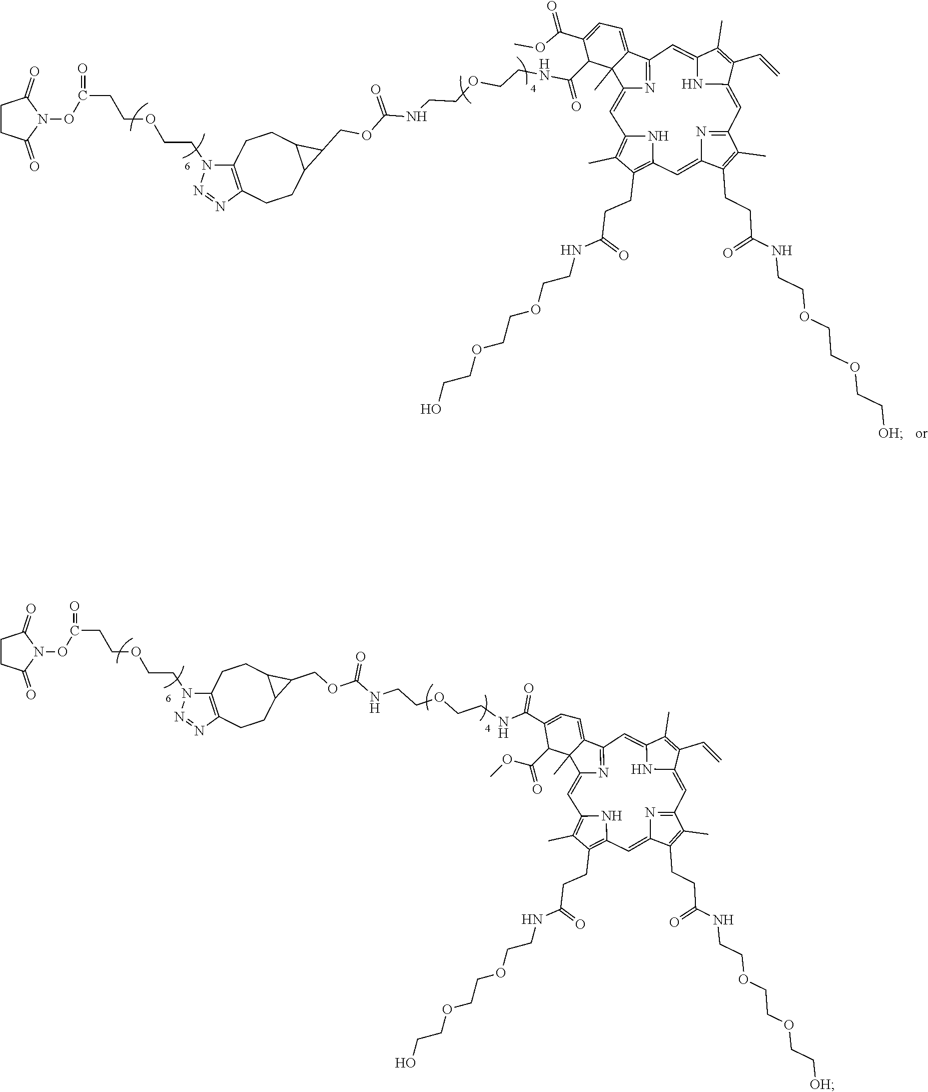
or a pharmaceutically acceptable salt thereof, a regioisomer, or a pharmaceutically acceptable salt of the regioisomer.

17. A conjugate comprising a benzoporphyrin analog covalently linked to a targeting agent, wherein the conjugate is represented by the following formula: 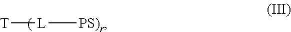
or a pharmaceutically acceptable salt thereof, wherein:
T is targeting agent;
L is a spacer connecting the targeting agent and PS;
r is an integer from 1 to 20;
PS is a photosensitizer that is a benzoporphyrin analog represented by the following formula: 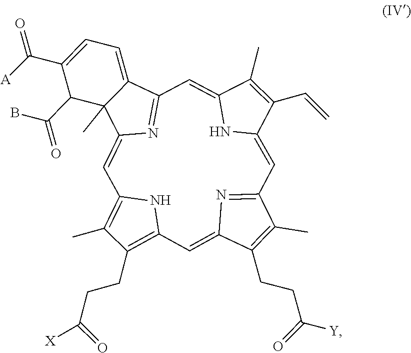
or a pharmaceutically acceptable salt thereof, wherein:
A, B, X and Y are each independently —OH, —OC₁₋₄alkyl, —N(R¹⁰⁰)(R¹⁰¹) or a bond covalently linked to L, provided one of A, B, X and Y is a bond covalently linked to L;
R¹⁰⁰ is H or C₁₋₃alkyl;
R¹⁰¹ is H, C₁₋₃alkyl or —(CH₂—CH₂—O)ₙᵣR¹⁰²;
R¹⁰² is H or Me;
nr is an integer from 1 to 16.

18. The conjugate of claim 17, or a pharmaceutically acceptable salt thereof, wherein -L-PS is represented by the following formula: 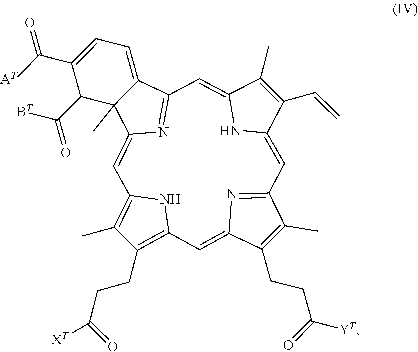
or a pharmaceutically acceptable salt thereof, wherein:
A^{T}, B^{T}, X^{T} and Y^{T} are each independently —OH, —OC₁₋₄alkyl, —N(R¹⁰⁰)(R¹⁰¹) or —Z-L^{P}-linker-L^{A}-R^{T}, provided one of A^{T}, B^{T}, X^{T} and Y^{T} is —Z-L^{P}-linker-L^{A}-R^{T};
Z is —O— or —NR¹—;
R¹ is H or C₁₋₃alkyl;
L^{P} and L^{A} are each independently a spacer;
Linker is connection moiety formed by a first reactive group attached to L and a second reactive group attached to L^{A}; and
R^{T} is a reactive group covalently linked to the targeting agent.

19. The conjugate of claim 17 or 18, or a pharmaceutically acceptable salt thereof, wherein:
(i) the targeting agent comprises a polypeptide that binds to the surface of a target cell;
(ii) the targeting agent is an antibody or an antigen-binding fragment thereof, optionally, wherein:
(a) the antibody or antigen-binding fragment thereof binds to an adipose cell, blood cell, cancer cell, endothelial cell, epithelial cell, immune cell, neuron, skin cell, stem cell, tumor cell, myeloid derived suppressor cell;
(b) the antibody or antigen-binding fragment thereof binds to a cancer-associated fibroblast, a tumor-associated macrophage, a T cell, or a regulatory T cell;
(c) the antibody or antigen-binding fragment thereof binds to EGFR/HER1, HER2, HER3, HER4, VEGF, VEGFR, VEGFR2, EpCAM, E-Cad, Folate Receptor alpha, Fibroblast activation protein (FAP), CD1c, CD2, CD3, CD4, CD5, CD6, CD7, CD8, CD11a, CD11b, CD11c, CD14, CD16, CD18, CD19, CD20, CD21, CD25, CD26, CD27, CD28, CD30, CD31, CD32, CD33, CD44, CD45, CD52, CD56, CD62L, CD64, CD66b, CD69, CD80, CD86, CD90, CD103, CD122, CD123, CD127, CD163, CD206, CD235a, CXCR6, MHC-II, CCR4, CCR5, CCR7, CLA, PD-1, PD-L1, CTLA-4, CEA, MUC-1, PSMA, Cancer antigen 125 (CA125), Alpha-fetoprotein (AFP), Lewis Y antigen, TAG-72, IL-13R, melanoma-associated antigen (MAGE)₁, MAGE2, MAGE3, MAGE4, tumor-associated glycoprotein 72 (TAG-72), gp100, p97 melanoma antigen, human milk fat globule (HMFG), melanoma antigen recognized by T cells 1 (MART1), B melanoma antigen (BAGE) 1, BAGE2, G antigen (GAGE) 1, GAGE2, GAGE3, GAGE4, GAGE5, GAGE6, breast cancer-associated DF3 antigen, New York esophageal squamous cell carcinoma 1 (NY-ESO-1), mesothelin, GITR, OX40, FR4, CXCR4, CCL4, Gr-1, IL-4Ra, IL-1Ra, CXCR2, or LAG-3; or
(d) the antibody or antigen-binding fragment thereof binds to CD2 or EGFR;
(iii) the targeting agent comprises a reactive group that can form a covalent bond with R^{T}, optionally, wherein;
(a1) the targeting agent comprises a reactive group selected from an amine group, a thiol group or an amide group; or
the targeting agent comprises the following group: 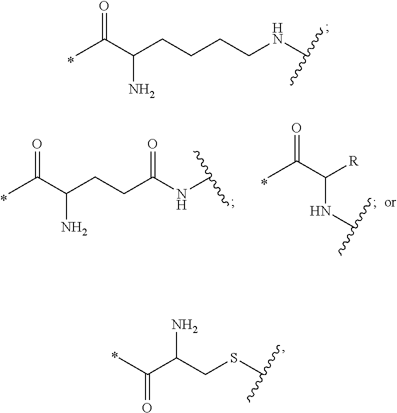
wherein R is side chain of an amino acid residue; *- represents a bond to the remaining targeting agent and 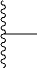
represents a bond to R^{T}; and/or
(a2) R^{T} is —C(═O)—, —NH—, or 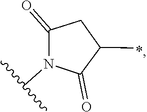 [SMILES: *N1C(=O)CC(C)C1=O]
wherein *- represents a bond to the targeting agent and 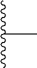
represents a bond to L^{A}; or
R^{T} is —C(═O)— or —NH—.

20. The conjugate of claim 18 or 19, or a pharmaceutically acceptable salt thereof, wherein:
(i) L^{A} is a spacer comprising a hydrophilic moiety;
(ii) L^{A} is a spacer comprising a PEG moiety:
(iii) L^{A} is *—CH₂CH₂(OCH₂CH₂)ₚ—, wherein * is the site connected to R^{T}; and p is an integer from 0 to 30, optionally, wherein, p is an integer from 2 to 16, from 2 to 10, from 2 to 8, or from 3 to 5; or p is 2, 3, 4, 5, 6, 7 or 8.

21. The conjugate of any one of claims 18-20, or a pharmaceutically acceptable salt thereof, wherein:
(i) the first reactive group and the second reactive group are each selected from a maleimide group, a thiol group, a cyclooctyne group, and an azido group; or
(ii) the Linker is represented by the following formula: 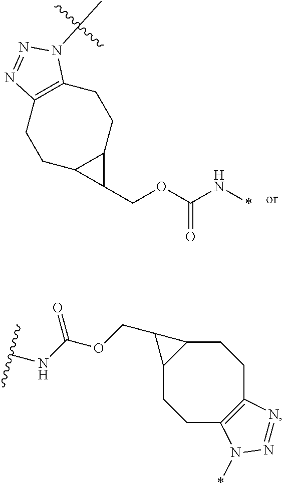 [SMILES: *NC(=O)OCC1C2CCc3nnn(*)c3CCC21 ; *NC(=O)OCC1C2CCc3nnn(*)c3CCC21]
wherein *- represents a bond to L^{P} and 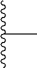
represents a bond to L^{A}.

22. The conjugate of any one of claims 18-21, or a pharmaceutically acceptable salt thereof, wherein:
a) A^{T} and B^{T} are —OCH₃; and one of X^{T} and Y^{T} is —OCH₃ and the other one of X^{T} and YT is —Z-L^{P}-linker-L^{A}-R^{T};
b) A^{T} and B^{T} are —OCH₃; and one of X^{T} and Y^{T} is —OH and the other one of X^{T} and YT is —Z-L^{P}-linker-L^{A}-R^{T};
c) one of A^{T} and B^{T} is —OCH₃, and the other one of A^{T} and B^{T} is —OH; and one of X^{T} and Y^{T} is —OH and the other one of X^{T} and Y^{T} is —Z-L^{P}-linker-L^{A}-R^{T};
d) A^{T} and B^{T} are —OH; and one of X and Y is —OH and the other one of X^{T} and Y^{T} is —Z-L^{P}-linker-L^{A}-R^{T}; or
e) one of A^{T} and B^{T} is —Z-L^{P}-linker-L^{A}-R^{T} and the other one of A^{T} and B^{T} is —OH or —OCH₃; and X^{T} and Y^{T} are both —NH(CH₂—CH₂—O)ₙᵣR¹⁰², wherein R¹⁰² is H or Me; and nr is an integer from 2 to 8.

23. The conjugate of claim 22, or a pharmaceutically acceptable salt thereof, wherein:
(i) -L-PS is represented by the following formula: 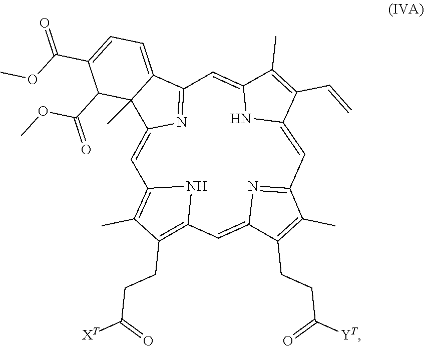
or a pharmaceutically acceptable salt thereof, wherein one of X^{T} and Y^{T} is —Z-L^{P}-linker-L^{A}-R^{T} and the other is —OH; or
(ii) -L-PS is represented by the following formula: 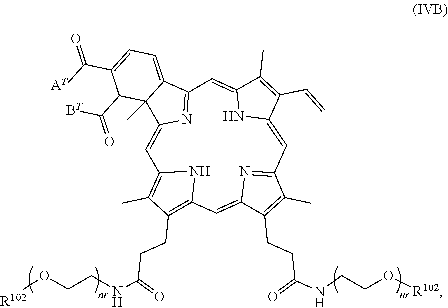
or a pharmaceutically acceptable salt thereof, wherein one of A^{T} and B^{T} is —Z-L^{P}-linker-L^{A}-R^{T} and the other is —OH or —OCH₃; R¹⁰² is H or Me; and nr is an integer from 2 to 8.

24. The conjugate of any one of claims 18-23, or a pharmaceutically acceptable salt thereof, wherein:
(i) Z is —NH—;
(ii) Z is —O—.

25. The compound of any one of claims 18-24, or a pharmaceutically acceptable salt thereof, wherein:
(i) L^{P} is a non-cleavable spacer, optionally, wherein:
(a) L^{P} comprises a hydrophilic moiety;
(b) L^{P} comprises a PEG moiety;
(c) L^{P} is *—CH₂CH₂—(OCH₂CH₂)ₘ—, wherein * is the site connected to ker, and m is an integer from 0 to 30, optionally, wherein m is an integer from 2 to 16, from 2 to 10, from 2 to 8, or from 3 to 5; or m is 2, 3, 4, 5, 6, 7 or 8; or
(d) L^{P} is *—CH₂CH₂—(OCH₂CH₂)₄—;
(ii) L^{P} is a cleavable spacer, optionally, wherein:
(a) the cleavable spacer comprises an enzyme labile group, a pH labile group, a disulfide group, a ROS cleavable group, a photocleavable group, optionally, wherein:
(1) the enzyme labile group is a group cleavable by an esterase, a peptidase, an aminopeptidase, a β-galactosidase, a β-glucuronidase, a carboxylesterase, a caspase, a diaphorase, a histone deacetylase, a legumain, or matrix metalloproteinase; or
(2) the enzyme labile group is a group cleavable by cathepsin B;
(ii) the spacer represented by L^{P} comprises a peptide and a self-immolative group;
(iii) the spacer L^{P} is presented by the following formula: 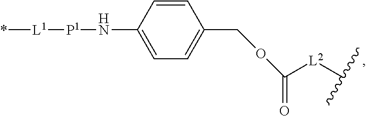
wherein:
*- represents a bond to the reactive group represented by ker; 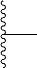
represents a bond to the group Z;
L¹ and L² are each independently a connecting spacer;
P¹ is a peptide comprising 2 to 5 amino acid residues, optionally, wherein:
(a1) P¹ is a peptide selected from Phe-Arg-Arg-Gly, Glu-Val-Cit, Val-Cit, Cit-Val, Gly-Gly-Phe, Val-Ala, and Ala-Val; or
P¹ is: 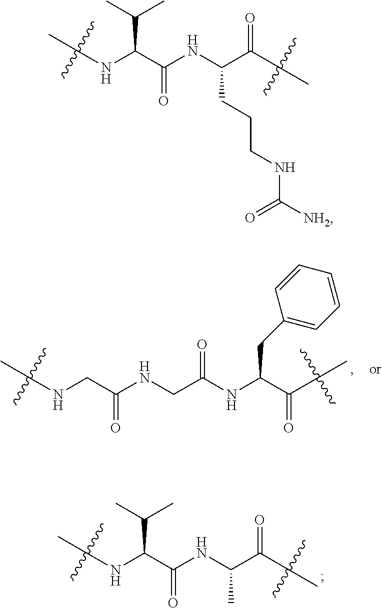 [SMILES: *N[C@H](C(=O)N[C@@H](CCCNC(N)=O)C(*)=O)C(C)C ; *NCC(=O)NCC(=O)N[C@@H](Cc1ccccc1)C(*)=O ; *N[C@H](C(=O)N[C@@H](C)C(*)=O)C(C)C]
and/or
(a2)^{L} comprises a hydrophilic moiety;
L¹ comprises a PEG moiety;
L¹ is *—CH₂—CH₂—(OCH₂CH₂)—C(═O)—**; n is an integer from 0 to 30; *- represents a bond to the reactive group represented by ker; and **- represents a bond to P¹, optionally, wherein n is an integer from 2 to 16, from 2 to 10, from 2 to 8, or from 3 to 5; or n is 2, 3, 4, 5, 6, 7 or 8; or
L¹ is *—CH₂—CH₂—(OCH₂CH₂)₃—C(═O)—**; and/or
(a3) L² is —NH—C₁₋₆alkyl-**, wherein ** is the site connected to Z; or
L² is —NH—CH₂CH₂—**; wherein ** is the site connected to Z.

26. The conjugate of claim 17, or a pharmaceutically acceptable salt thereof, wherein -L-PS is represented by the following formula: 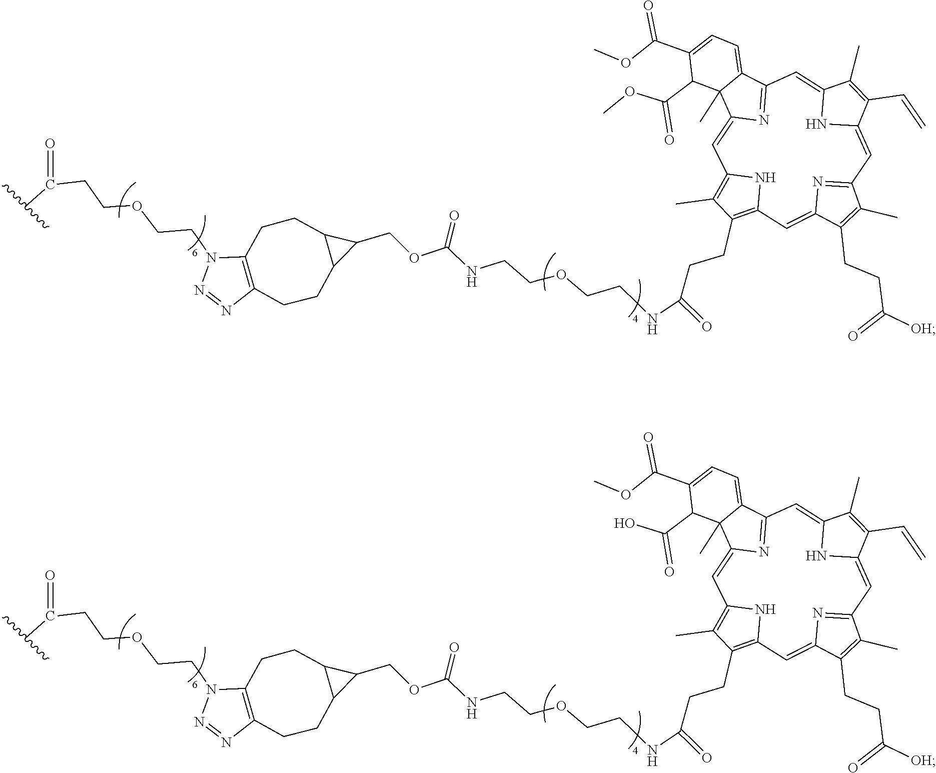 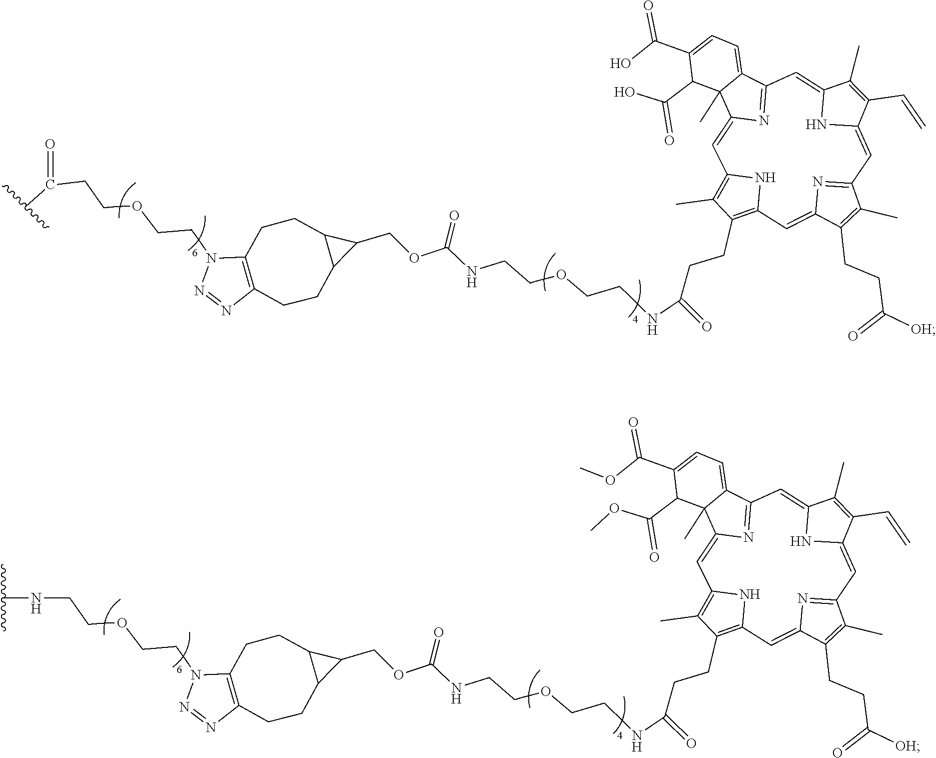 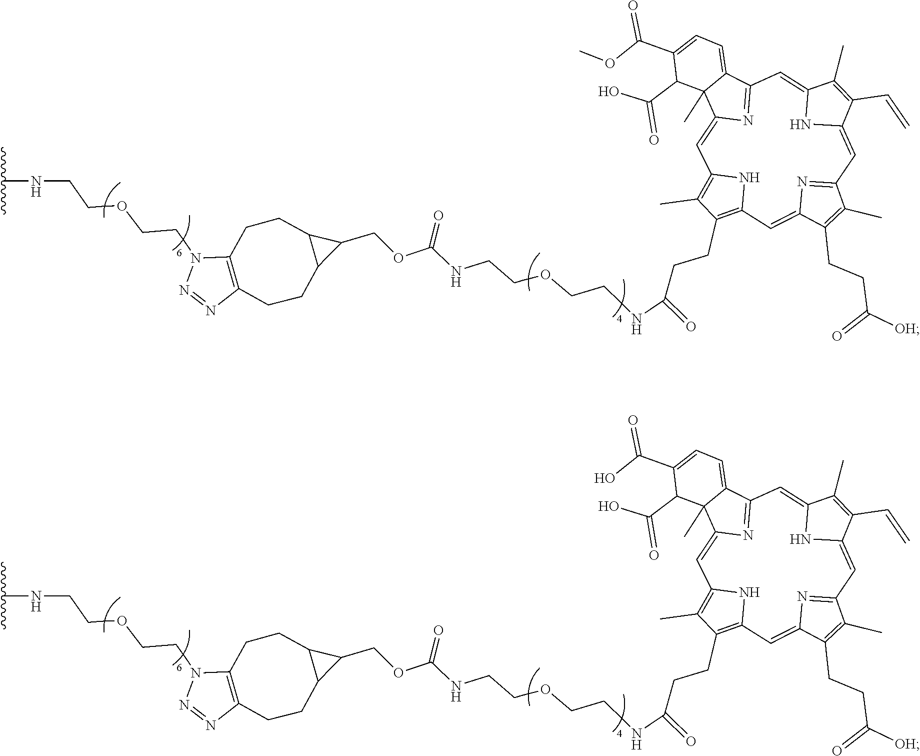 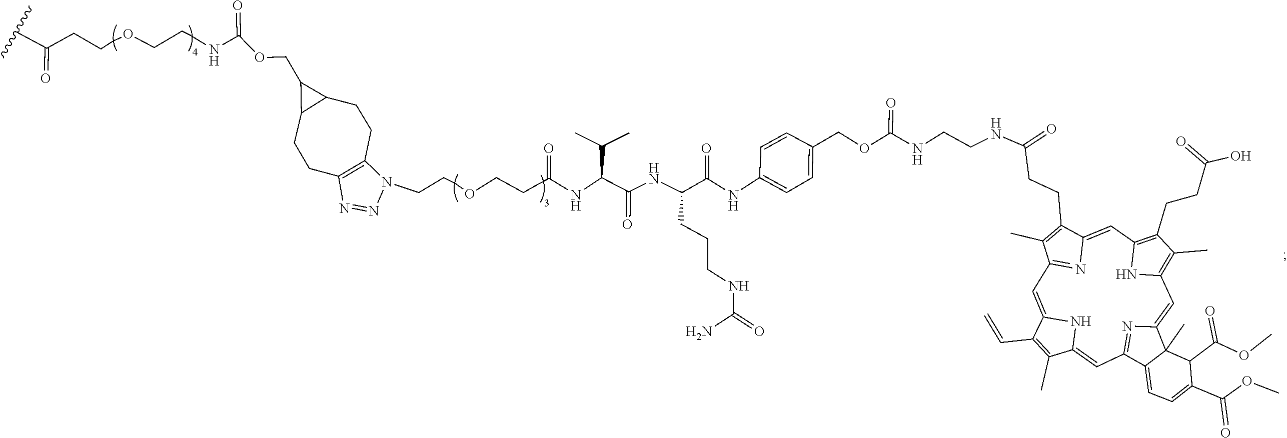 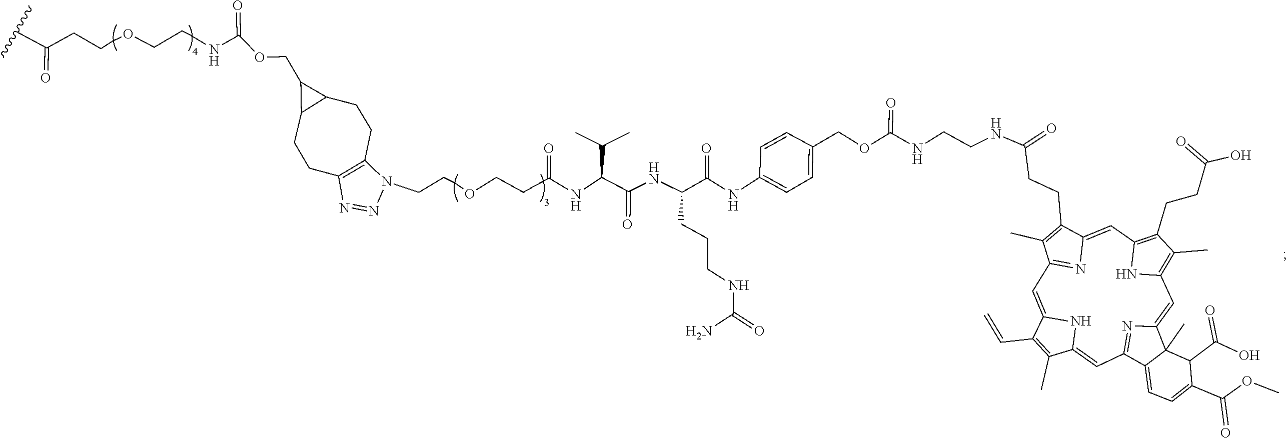 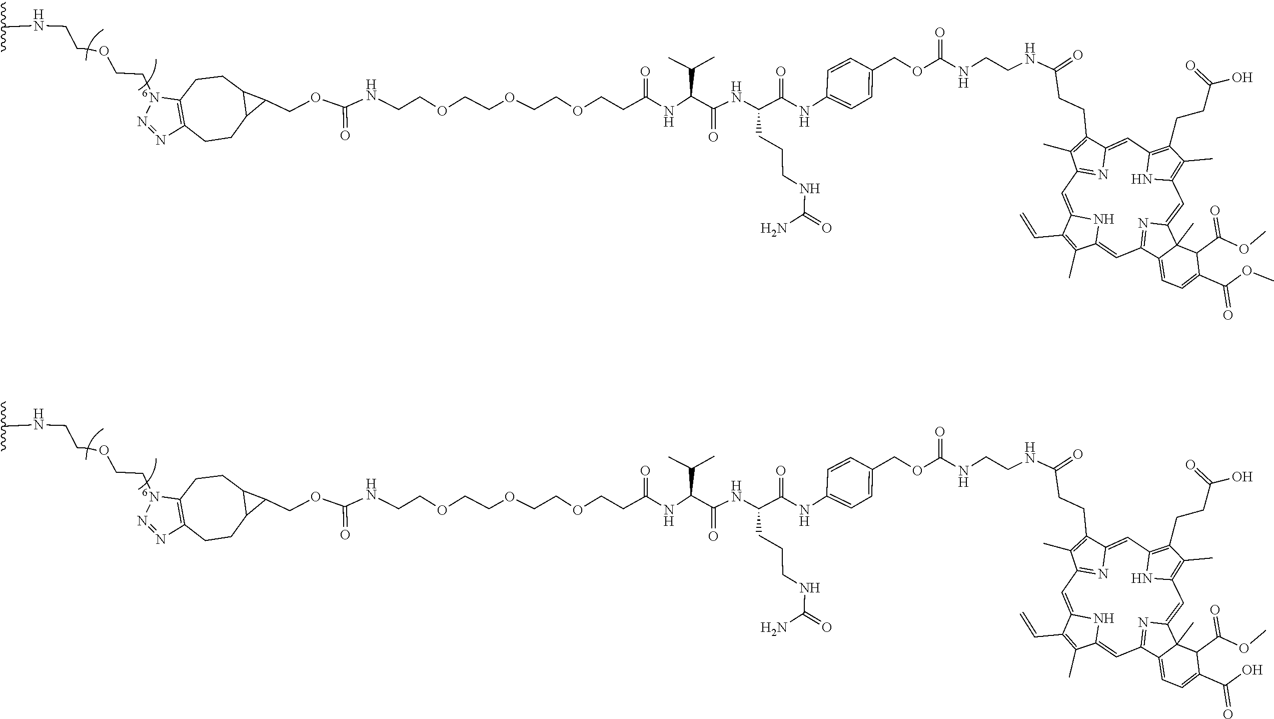 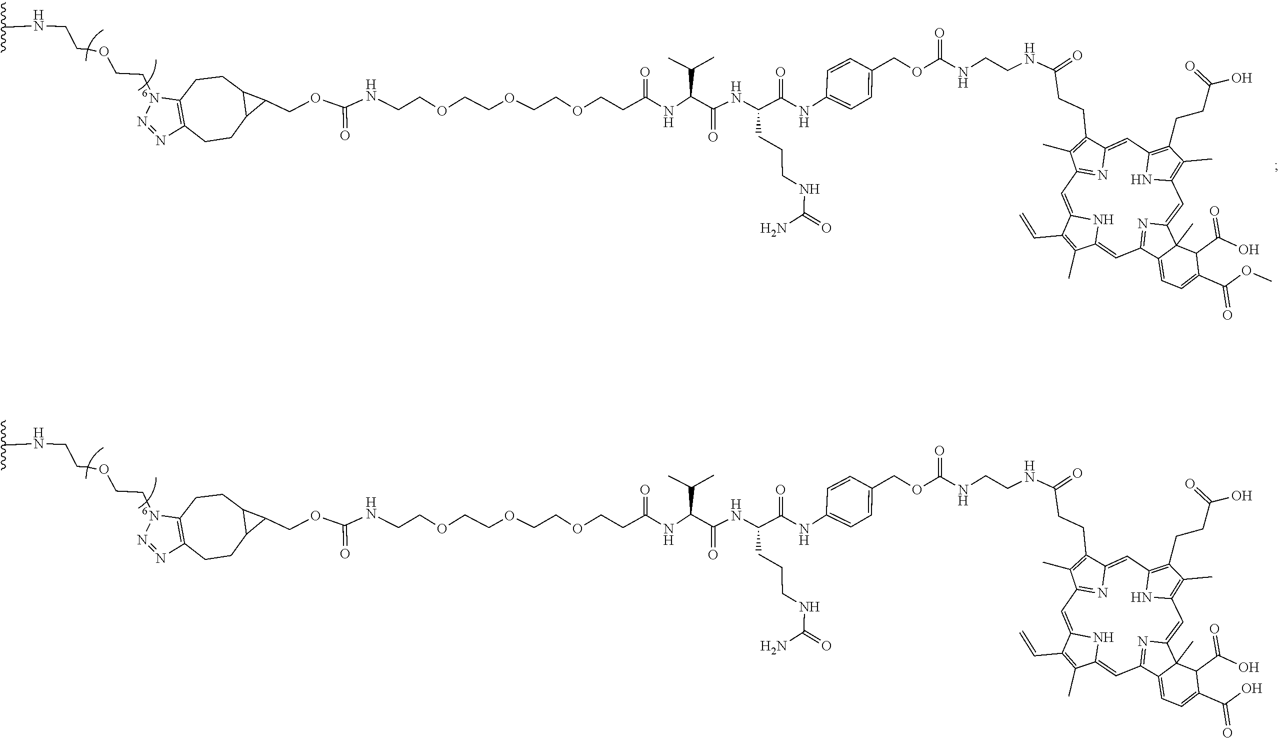 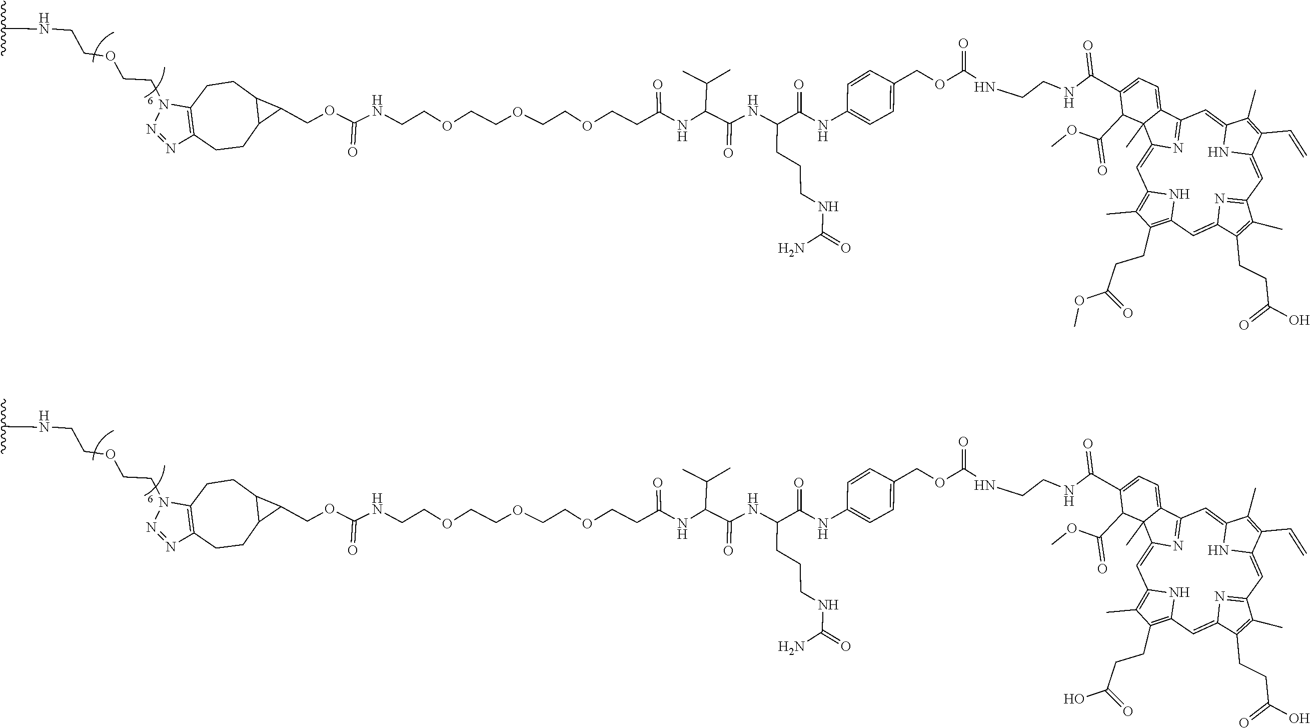 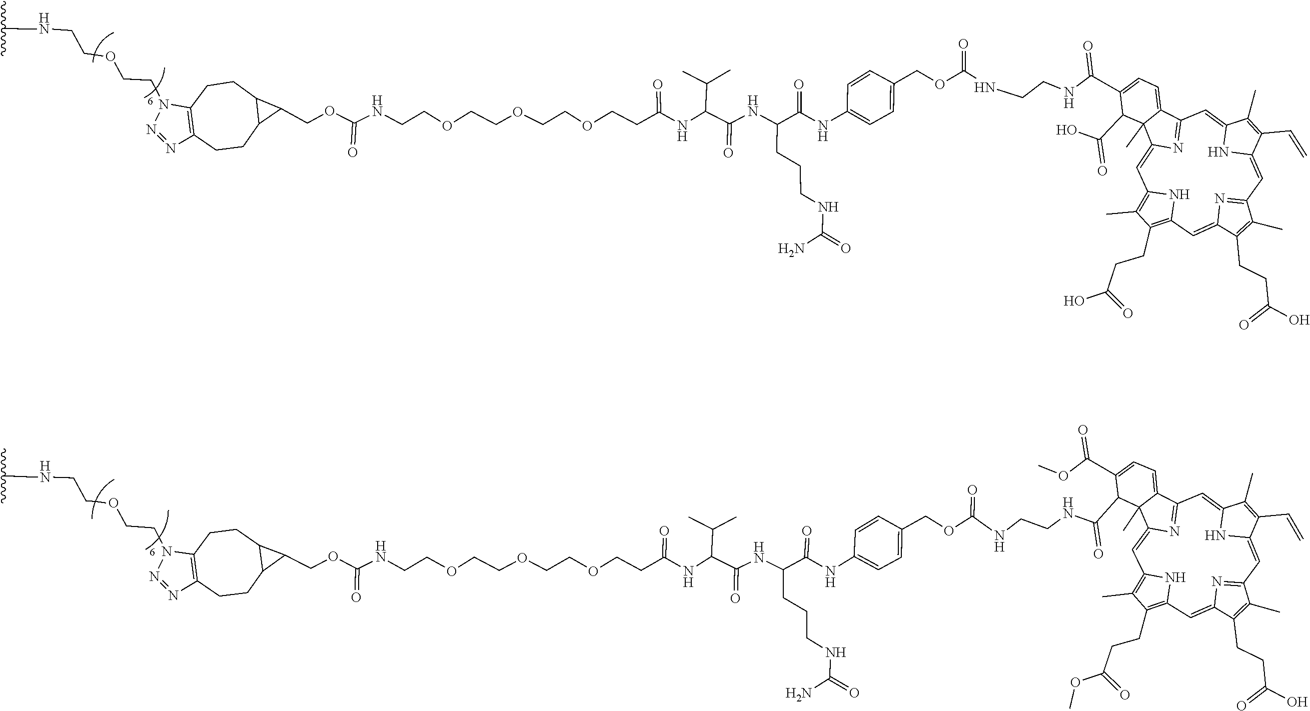 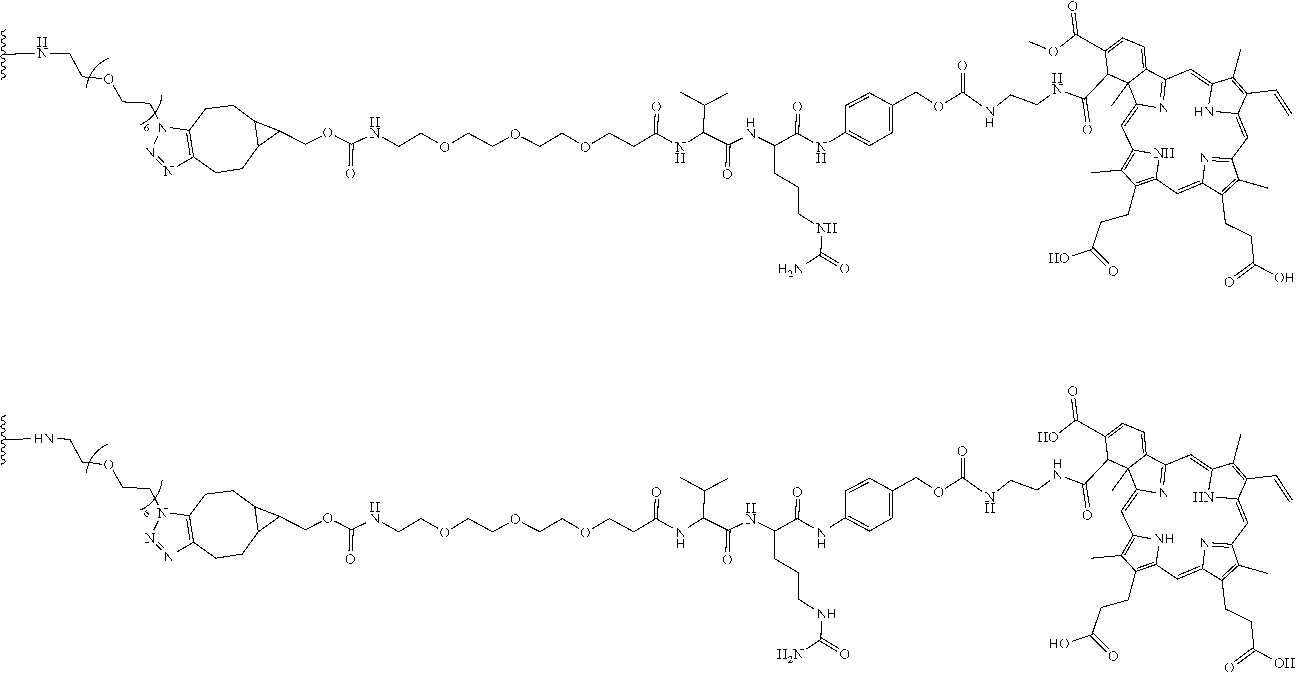 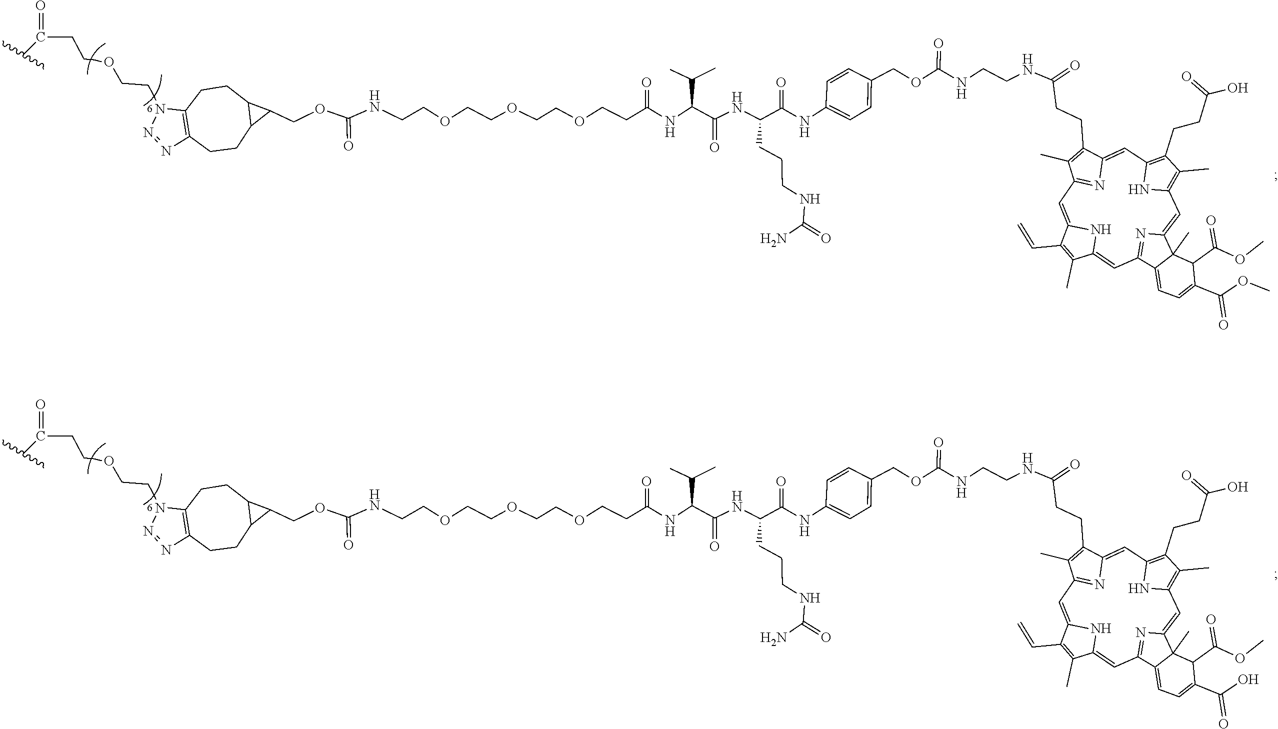 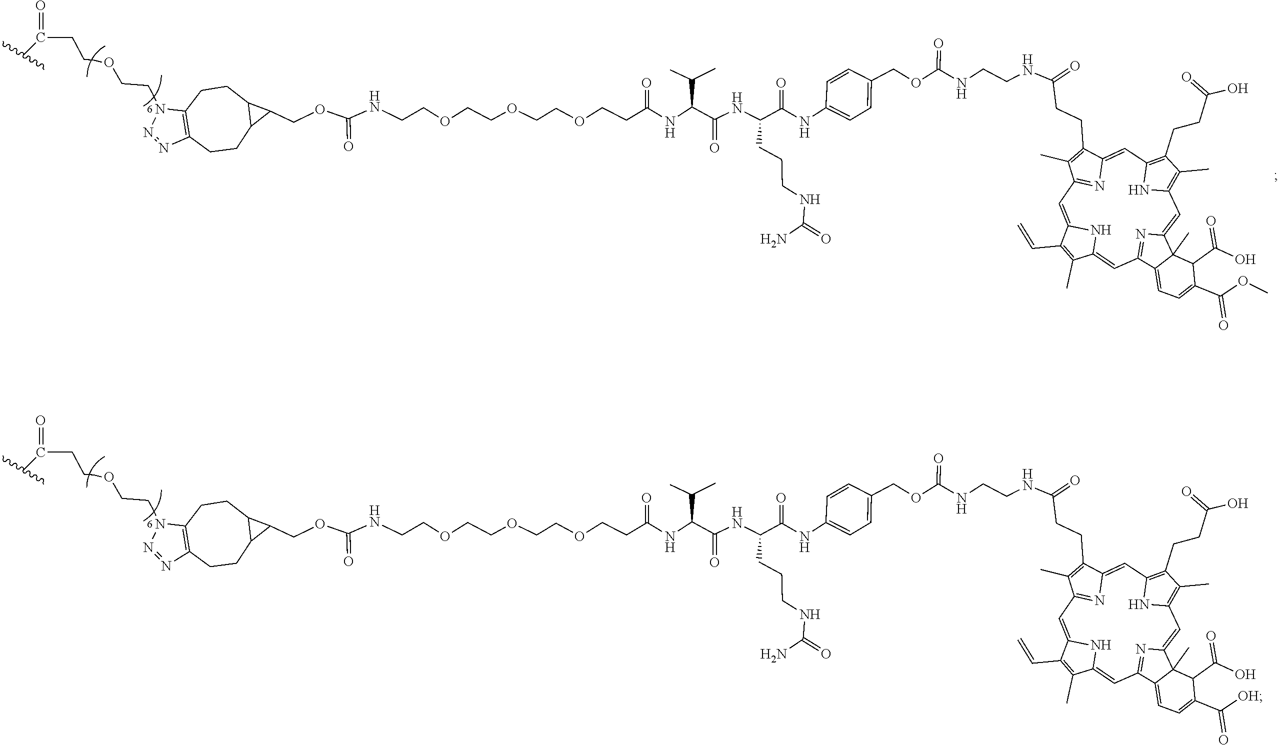 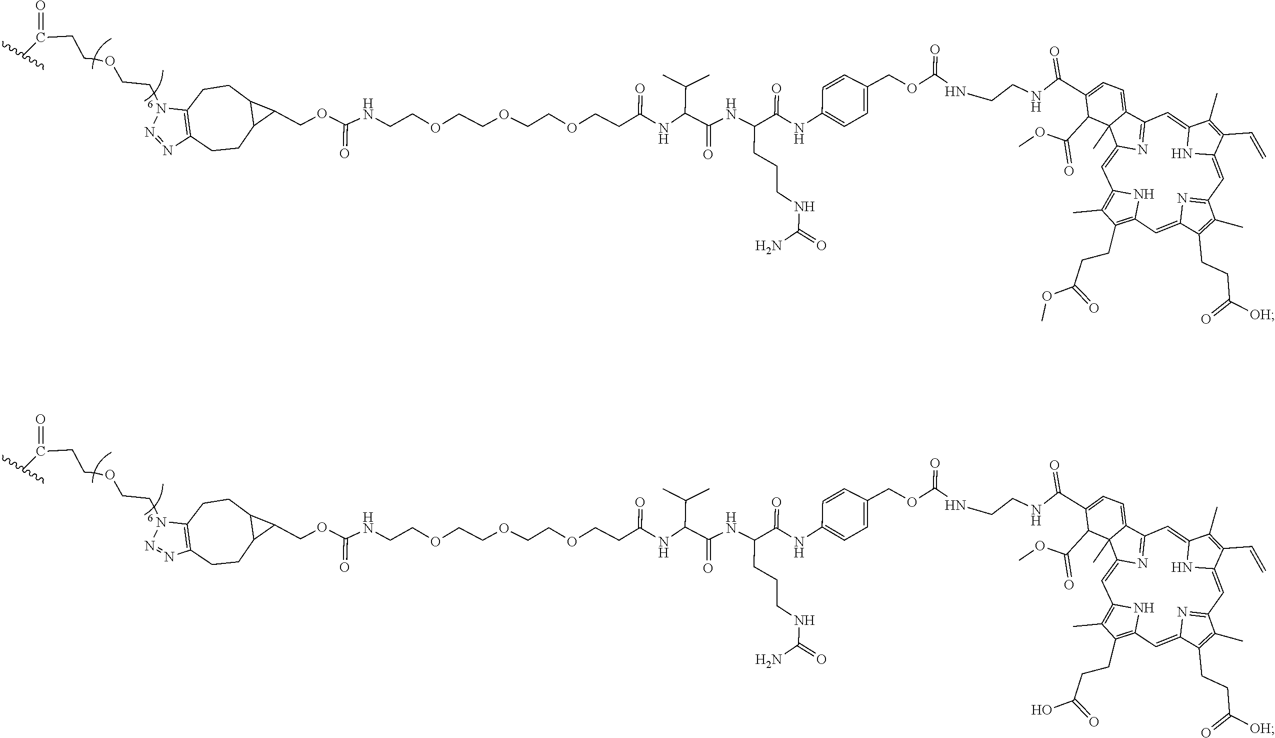 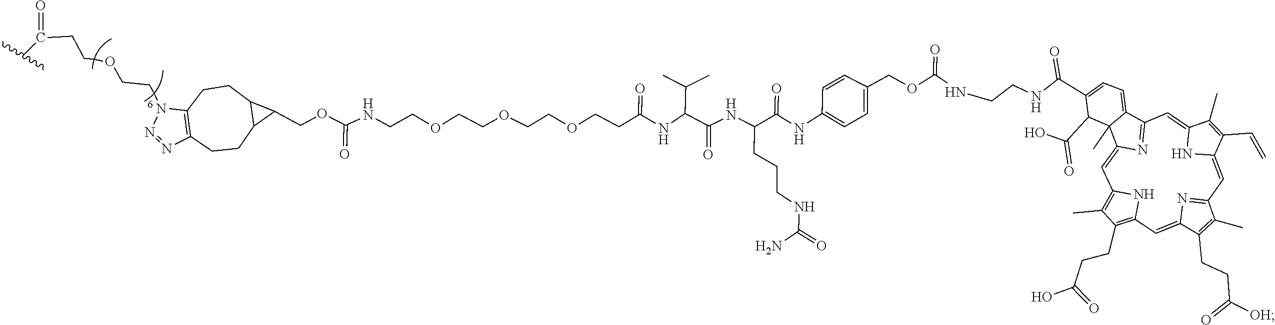 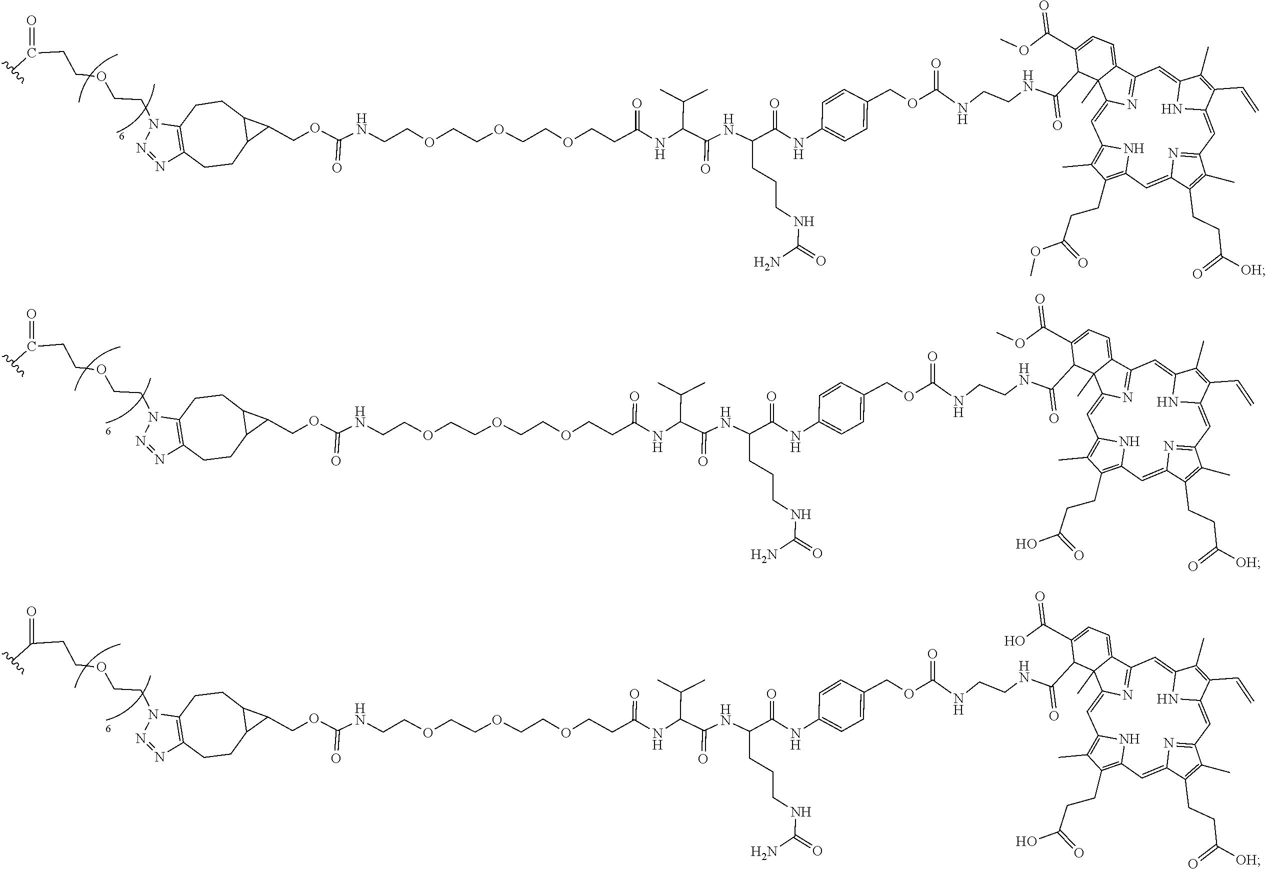 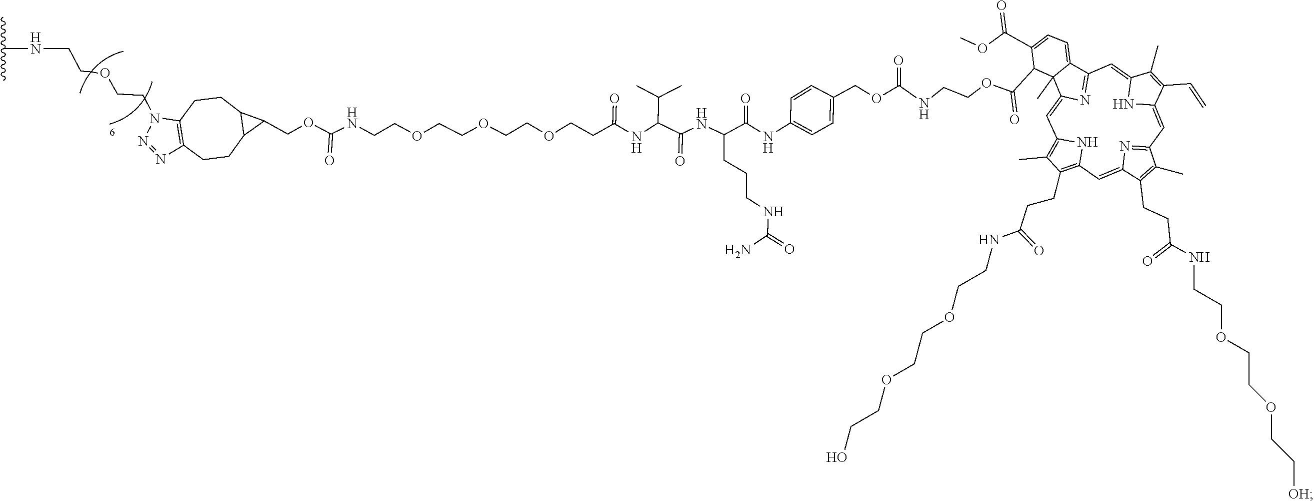 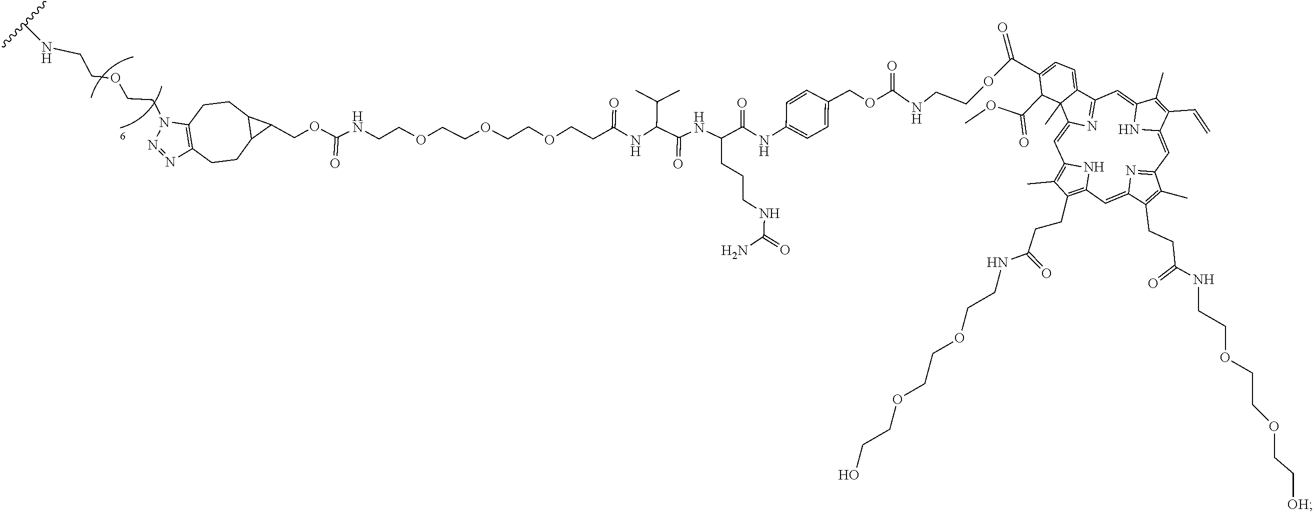 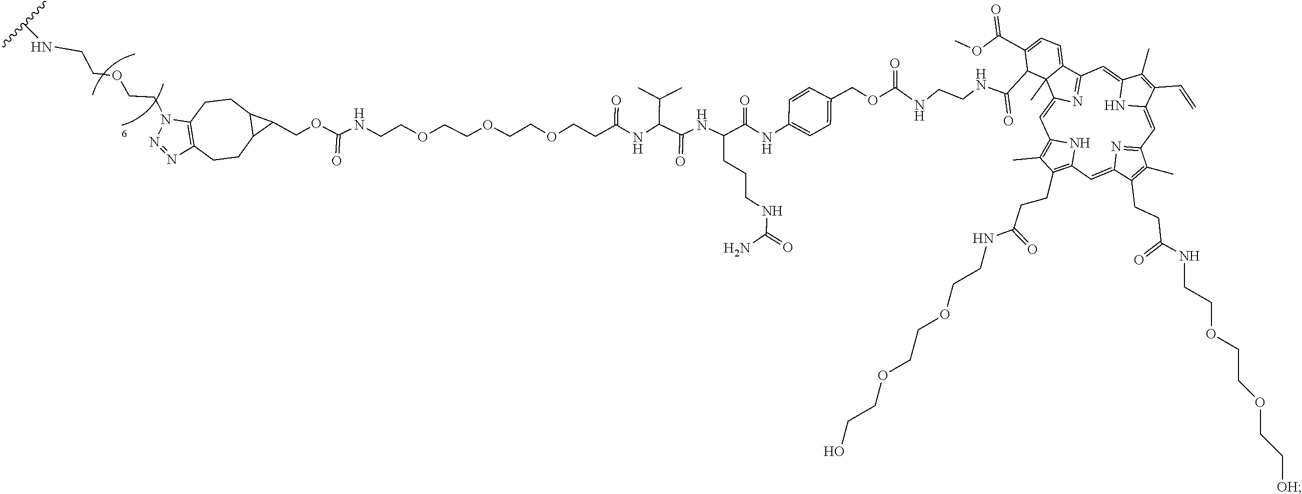 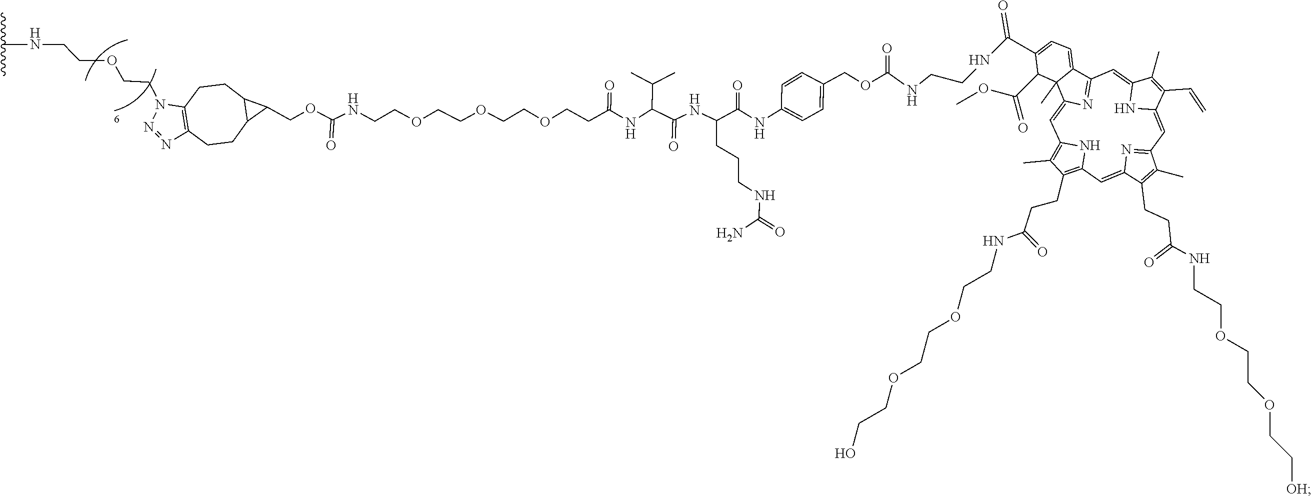 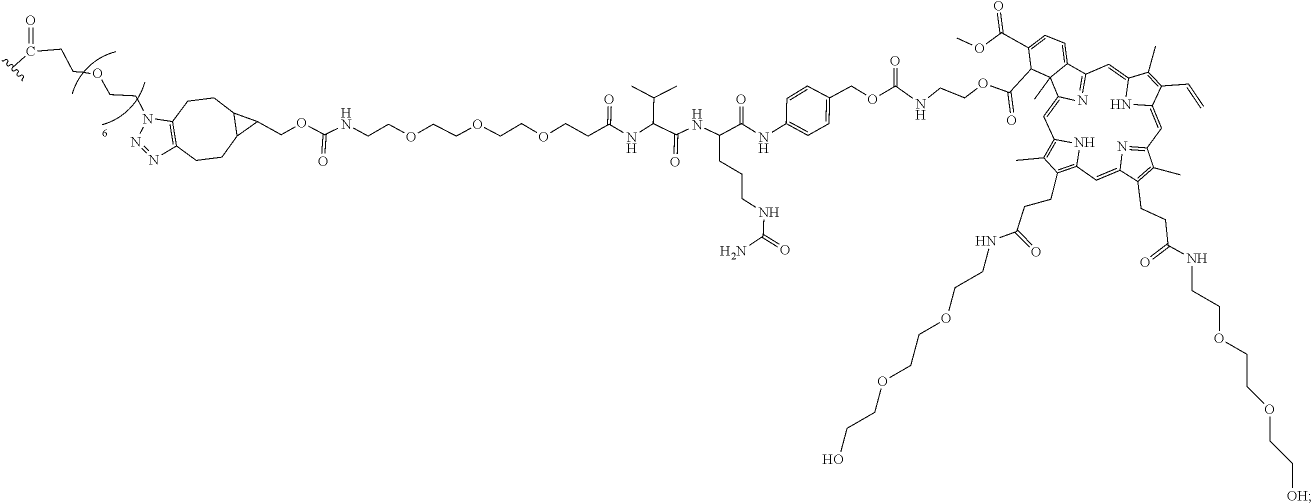 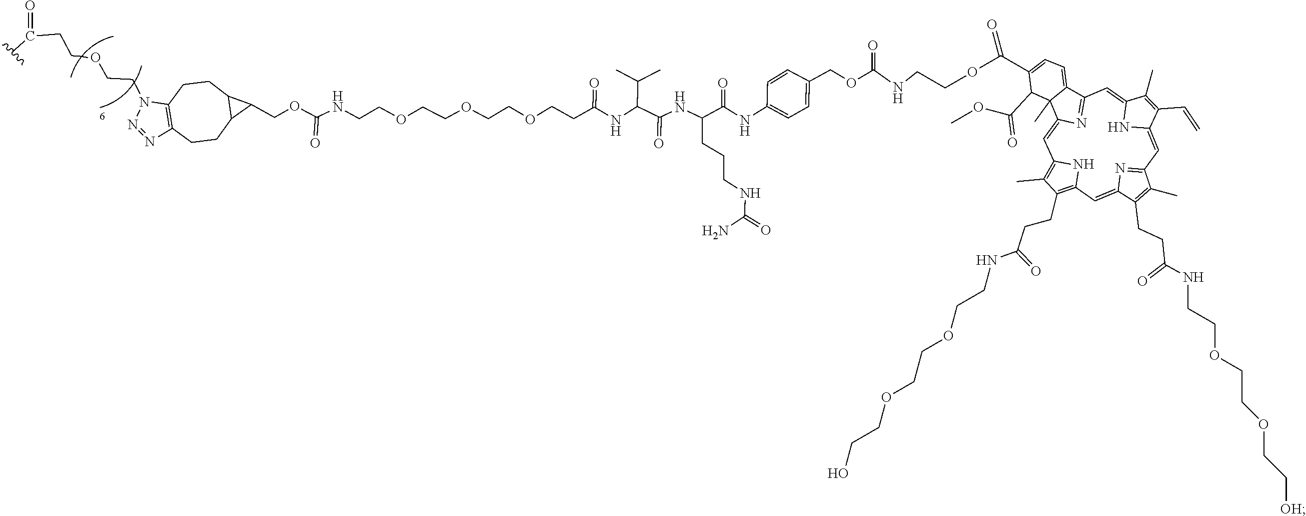 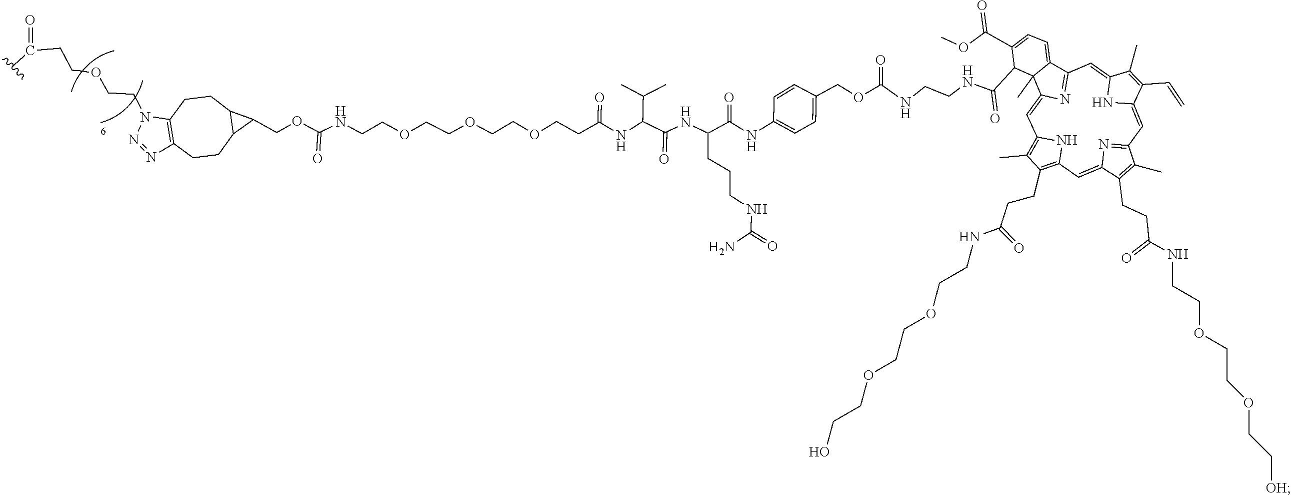 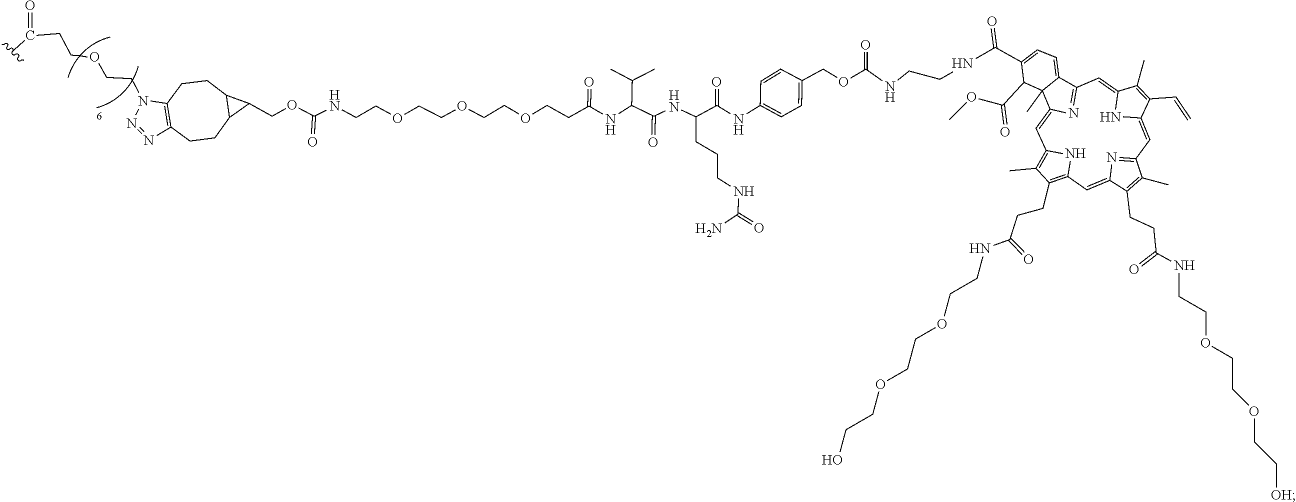 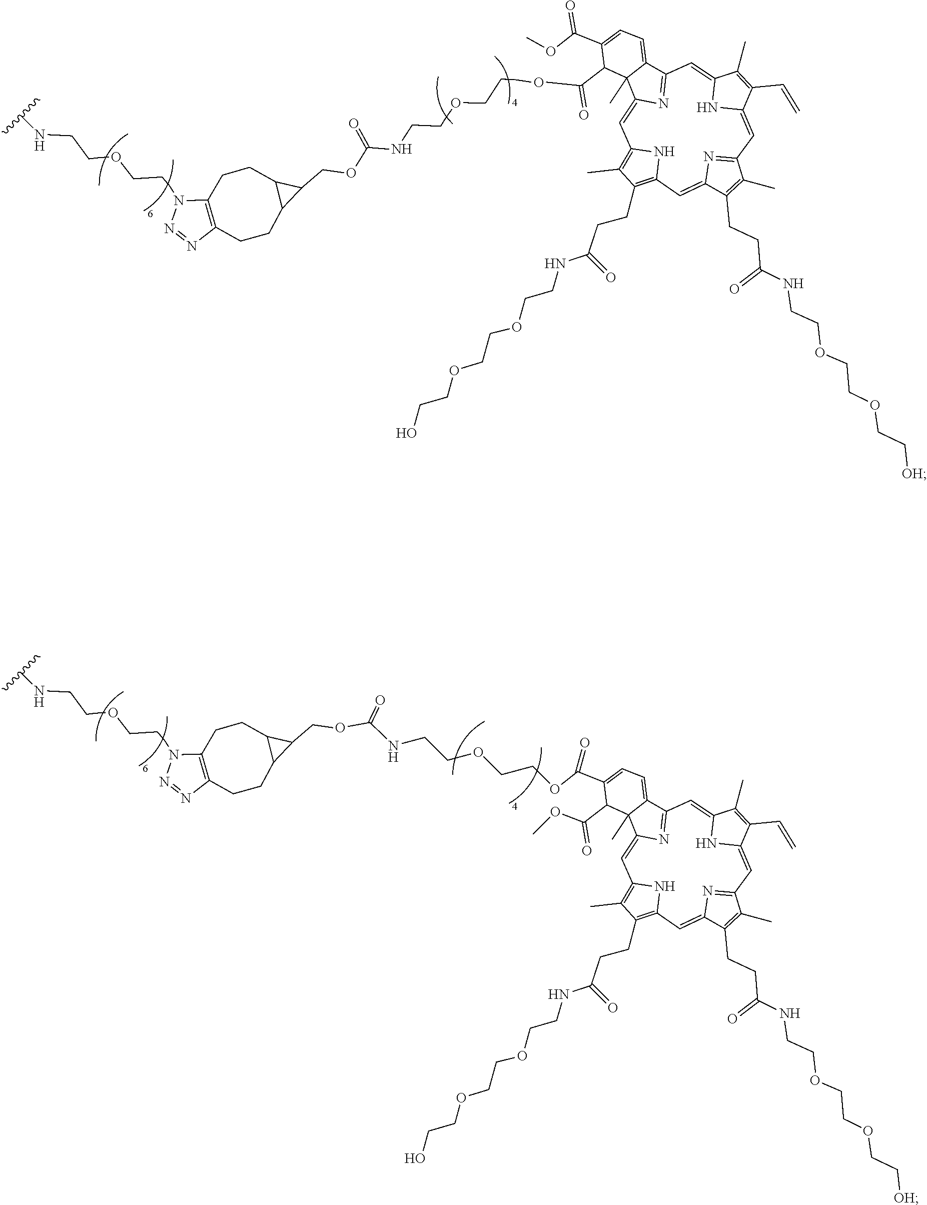 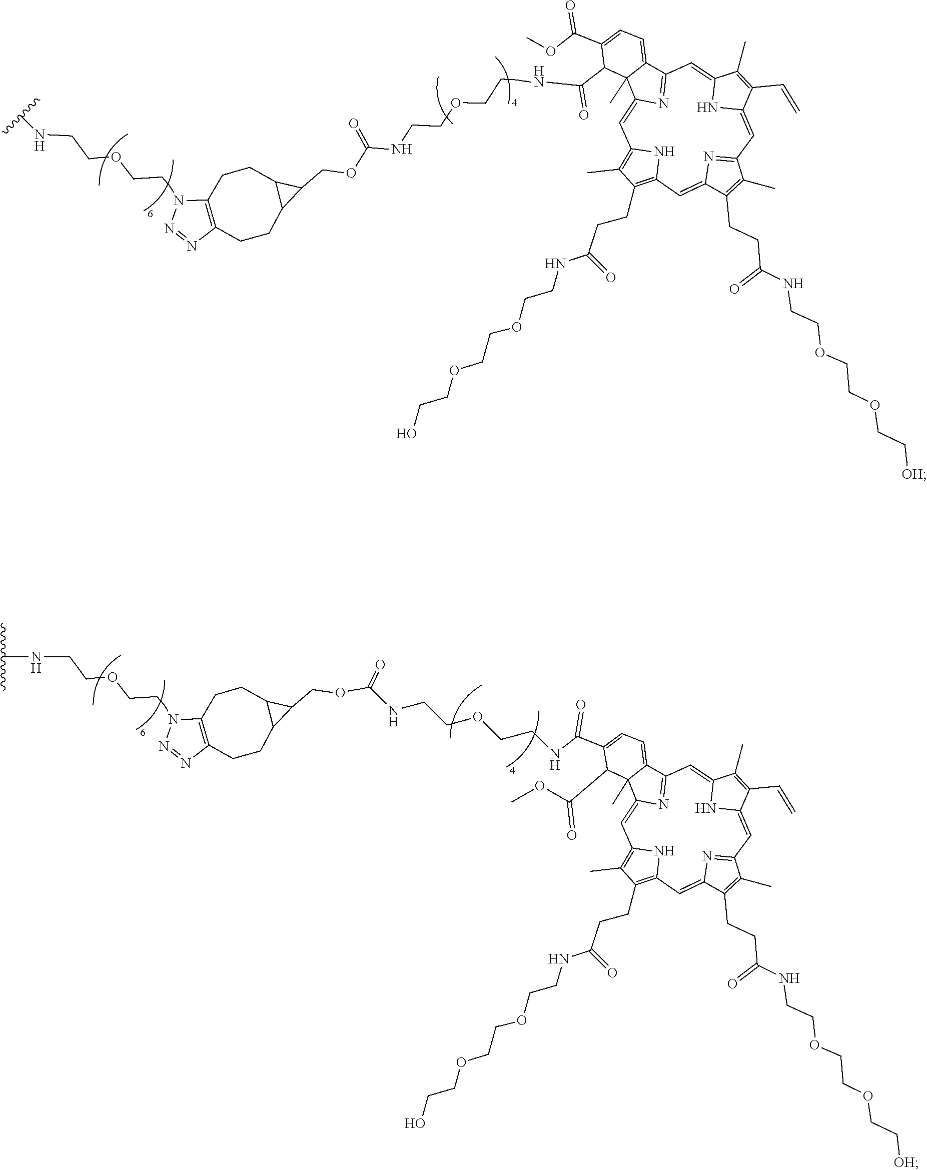 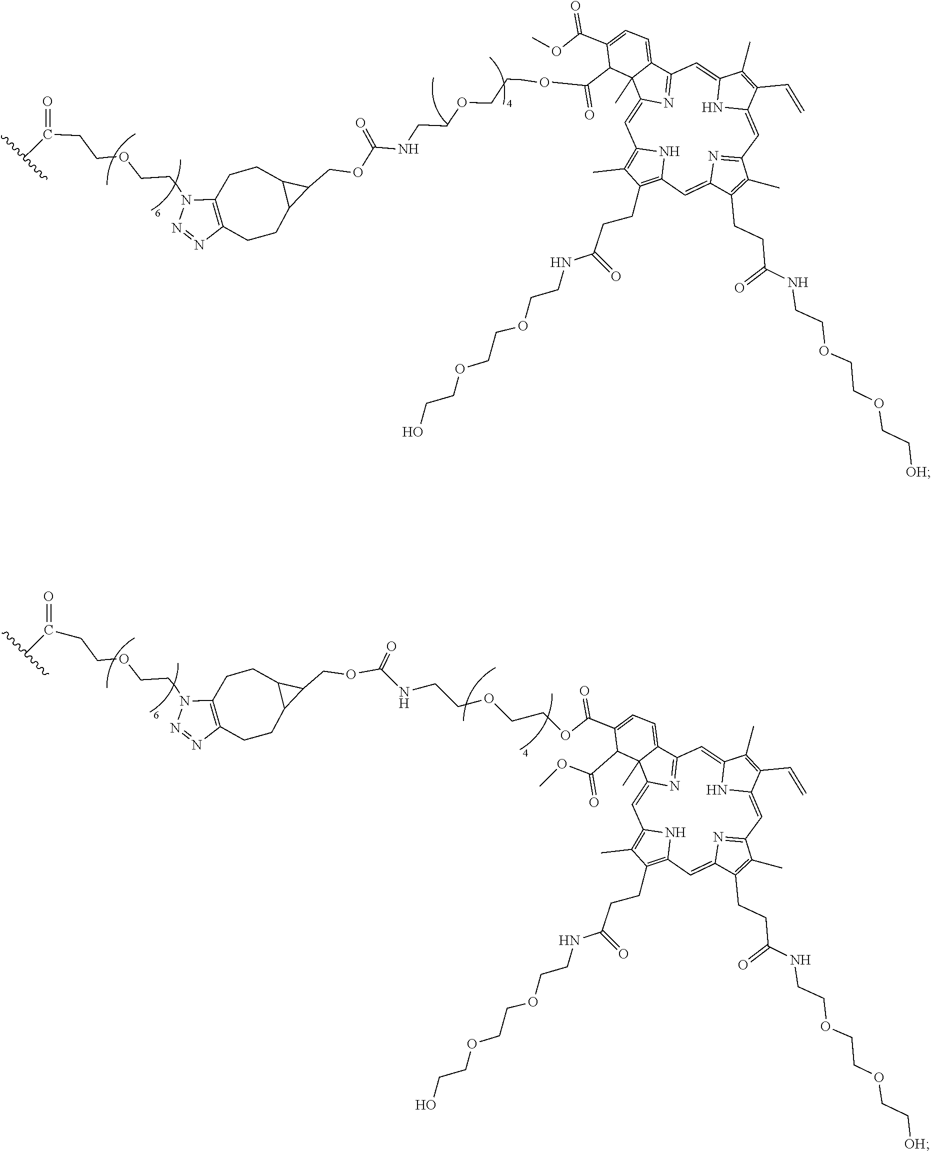 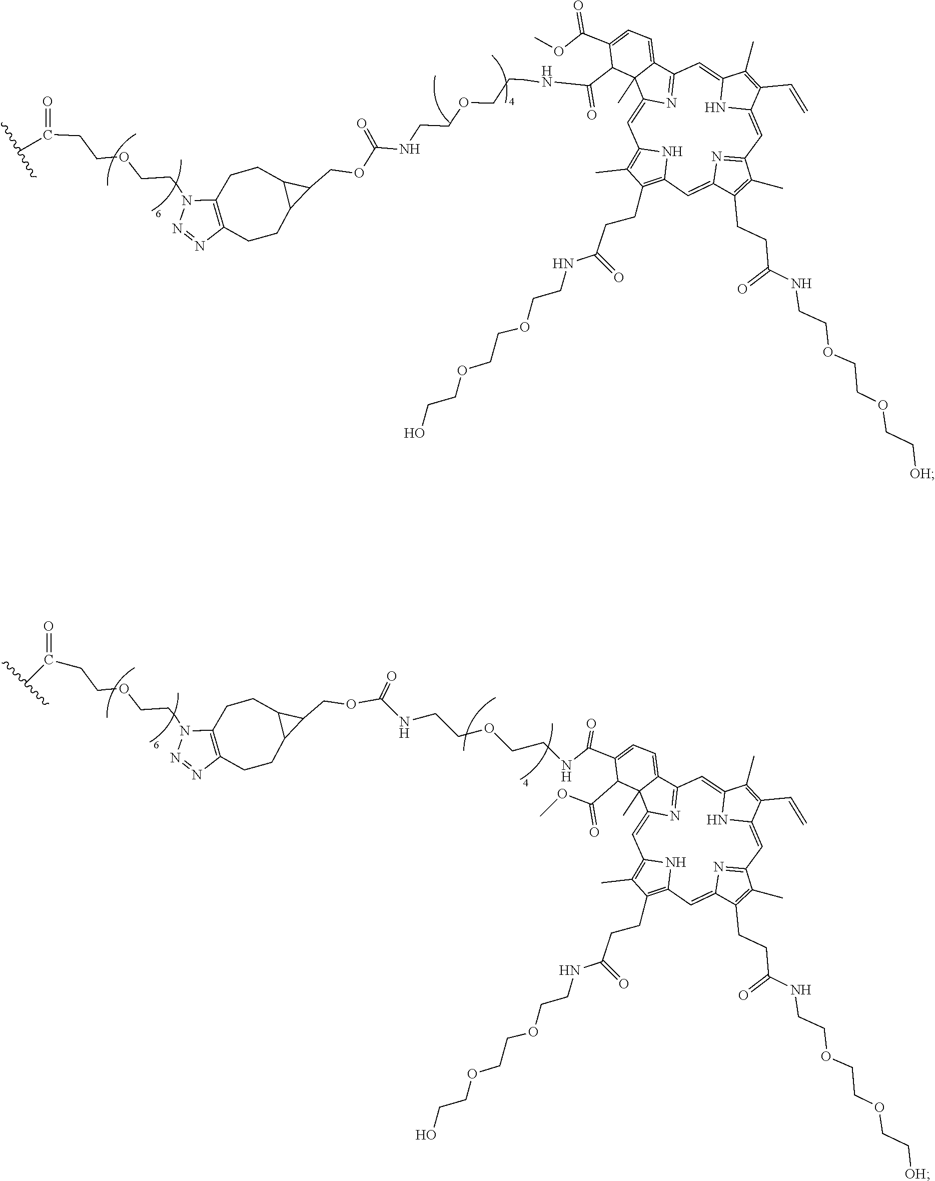 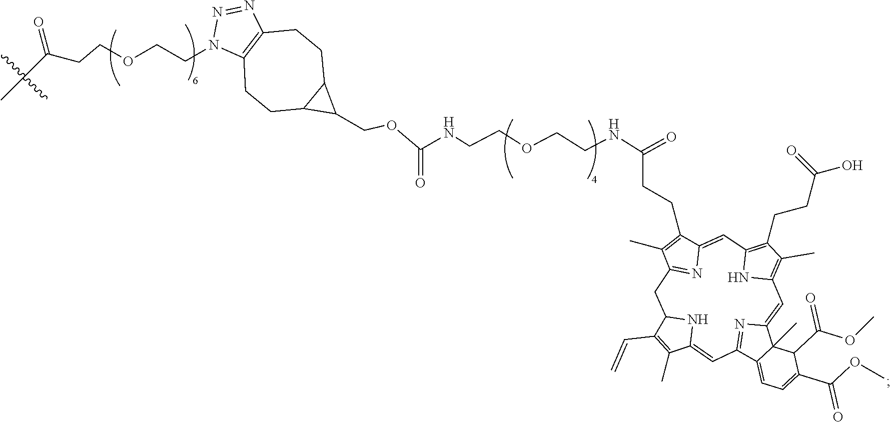 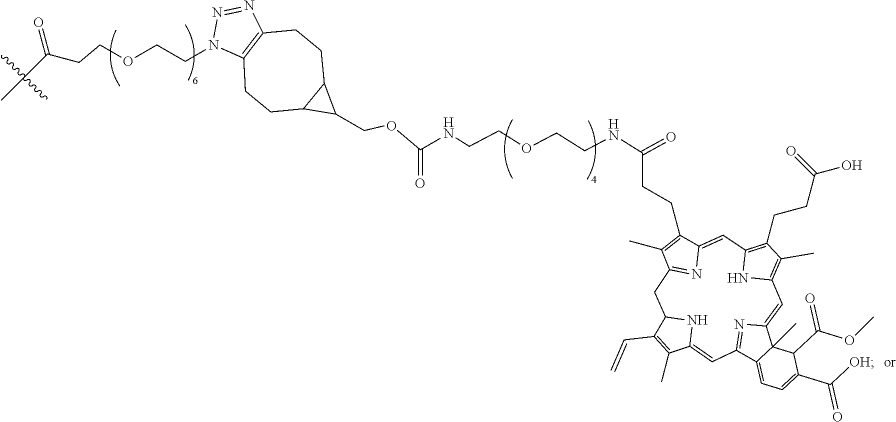 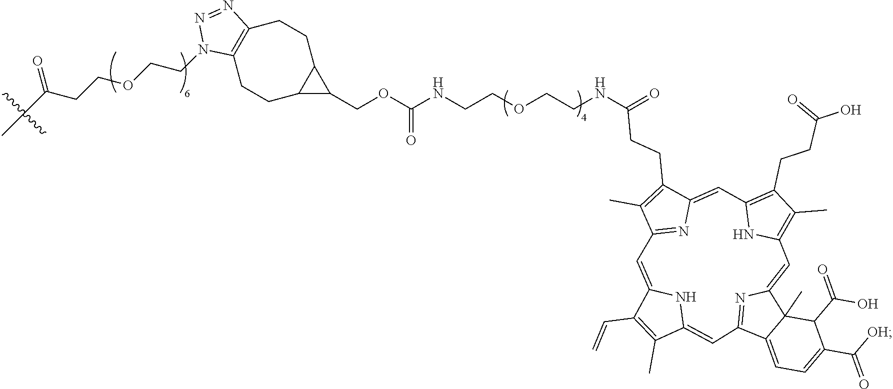
or a pharmaceutically acceptable salt thereof, wherein 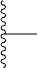
indicates a bond to the targeting agent.

27. A pharmaceutical composition comprising a conjugate of any one of claims 17-26, or a pharmaceutically acceptable salt thereof, and a pharmaceutically acceptable excipient.

28. A method of treating a subject having a disease or condition comprising:
a) administering to the subject a therapeutically effective amount of the conjugate of any one of claims 17-26, or a pharmaceutically acceptable salt thereof, or a pharmaceutical composition of claim 27; and
b) after administering the conjugate or a pharmaceutically acceptable salt thereof or a pharmaceutical comprising the conjugate or a pharmaceutically acceptable salt thereof, illuminating a target region of the subject with a near infrared (NIR) light sufficient to activate the photosensitizer of the conjugate, optionally, wherein:
(ia) the NIR light is at a wavelength between 600 nm to 850 nm; or
the NIR light is at a wavelength between 660 nm to 740 nm; and/or
(ib) the illumination is performed at least 5 minutes after administration of the conjugate; or
the illumination is performed between 1 hour and 48 hours, between 4 hours and 30 hours or between 8 hours and 24 hour after administration of the conjugate.

29. The method of claim 28, wherein:
(i) the target region is a tumor cell, a mass of tumor cells, a solid tumor, in the vicinity of a solid tumor, a metastasis, a metastasized tumor cell, in the vicinity of a metastasis or a pre-cancerous lesion;
(ii) the target region is skin lesions;
(iii) the target region is kidney of the subject;
(iv) the target region is gut or bowel of the subject; or
(v) the target region is inflamed joints of the subject.

30. The method of claim 28 or 29, wherein:
(i) the disease or condition is a cancer, optionally, wherein the cancer is selected from the group consisting of colon cancer, colorectal cancer, pancreatic cancer, breast cancer, skin cancer, lung cancer, non-small cell lung carcinoma, renal cell carcinoma, thyroid cancer, prostate cancer, head and neck cancer, esophogeal, gastrointestinal cancer, stomach (gastric) cancer, cancer of the small intestine, colon cancer, spindle cell neoplasm, hepatic carcinoma, liver cancer, cholangiocarcinoma, cancer of peripheral nerve, brain cancer, cancer of skeletal muscle, cancer of smooth muscle, bone cancer, cancer of adipose tissue, cervical cancer, uterine cancer, cancer of genitals, lymphoma, and multiple myeloma;
(ii) the disease or condition is an inflammatory or autoimmune condition/disease;
(iii) the disease or condition is skin lesions associated with psoriasis, atopic dermatitis, lupus, vitiligo, graft-versus-host disease, cutaneous T-cell lymphoma, contact dermatitis, cutaneous hypersensitivity response, lichen planus, lichen planopilaris, rejection of vascularized composite allografts, alopecia areata, scarring alopecia or sarcoid;
(iv) the disease or condition is T cell mediated kidney or renal pathology/disease associated with lupus nephritis, autoimmune nephritis or kidney graft rejection;
(v) the disease or condition is gastrointestinal inflammation as a result of an autoimmune and/or an inflammatory condition, e.g., inflammatory bowel disease (Crohn's disease, ulcerative colitis);
(vi) the disease or condition is joint inflammation from rheumatoid arthritis or spondyloarthritides.

31. A method of imaging a cell or tissue having a target molecule in a subject, the method comprising:
a) administering to a subject the conjugate of any one of claims 17-26, or a pharmaceutically acceptable salt thereof, or a pharmaceutical composition of claim 27; and
b) after administering the conjugate or a pharmaceutically acceptable salt thereof or a pharmaceutical comprising the conjugate or a pharmaceutically acceptable salt thereof, illuminating a target region of the subject with a near infrared (NIR) light sufficient to activate the photosensitizer of the conjugate, thereby providing an image of the presence of the target molecule associated with the cell or tissue.

32. A compound represented by the following formula: 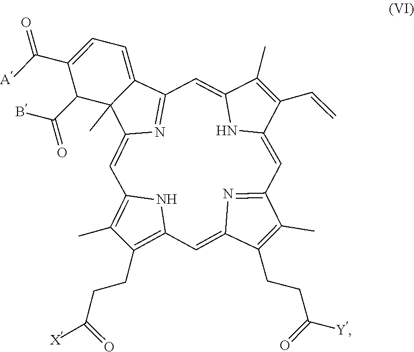
or a pharmaceutically acceptable salt thereof, wherein:
A′, B′, X′ and Y′ are each independently —OH, —OC₁₋₄alkyl, —N(R¹⁰⁰)(R¹⁰¹), or —Z—C₁₋₆allkylene-NH₂, provided one of A′, B′, X′ and Y′ is —Z—C₁₋₆allkylene-NH₂;
R¹⁰⁰ is H or C₁₋₃alkyl;
R¹⁰¹ is H, C₁₋₃alkyl or —(CH₂—CH₂—O)ₙᵣR¹⁰²;
R¹⁰² is H or Me;
nr is an integer from 1 to 16;
Z is —O— or —NR¹—; and
R¹ is H or C₁₋₃alkyl.

33. The compound of claim 32, or a pharmaceutically acceptable salt thereof, wherein:
a) A′ and B′ are —OCH₃; and one of X′ and Y′ is —OCH₃ and the other one of X′ and Y′ is —Z—C₁₋₆allkylene-NH₂;
b) A′ and B′ are —OCH₃; and one of X′ and Y′ is —OH and the other one of X′ and Y′ is —Z—C₁₋₆allkylene-NH₂
c) one of A′ and B′ is —OCH₃, and the other one of A′ and B′ is —OH; and one of X′ and Y′ is —OH and the other one of X′ and Y′ is —Z—C₁₋₆allkylene-NH₂;
d) A′ and B′ are —OH; and one of X′ and Y′ is —OH and the other one of X′ and Y′ is —Z—C₁₋₆allkylene-NH₂; or
e) one of A′ and B′ is —OCH₃ and the other one of A′ and B′ is —Z—C₁₋₆allkylene-NH₂; and X′ and Y′ are both —NH(CH₂—CH₂—O)ₙᵣR¹⁰², wherein R¹⁰² is H or Me; and nr is an integer from 2 to 8.

34. The compound of claim 32, or a pharmaceutically acceptable salt thereof, wherein:
(i) the compound is represented by the following formula: 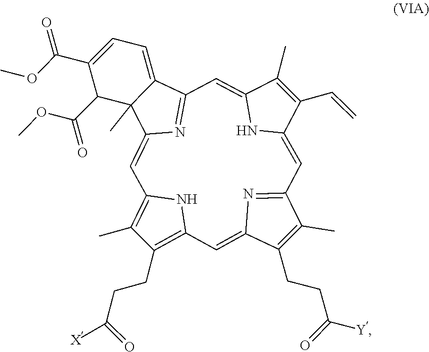
or a pharmaceutically acceptable salt thereof, wherein one of X′ and Y′ is —Z—C₁₋₆allkylene-NH₂ and the other is —OH; or
(ii) the compound is represented by the following formula: 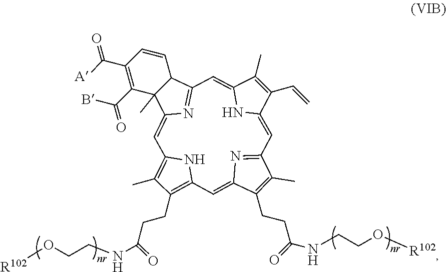
or a pharmaceutically acceptable salt thereof, wherein one of A′ and B′ is —Z—C₁₋₆allkylene-NH₂ and the other is —OH or —OCH₃.

35. The compound of any one of claims 32-34, or a pharmaceutically acceptable salt thereof, wherein:
(i) Z is —NH—; or
(ii) Z is —O—.

36. The compound of claim 32, or a pharmaceutically acceptable salt thereof, wherein the compound is: 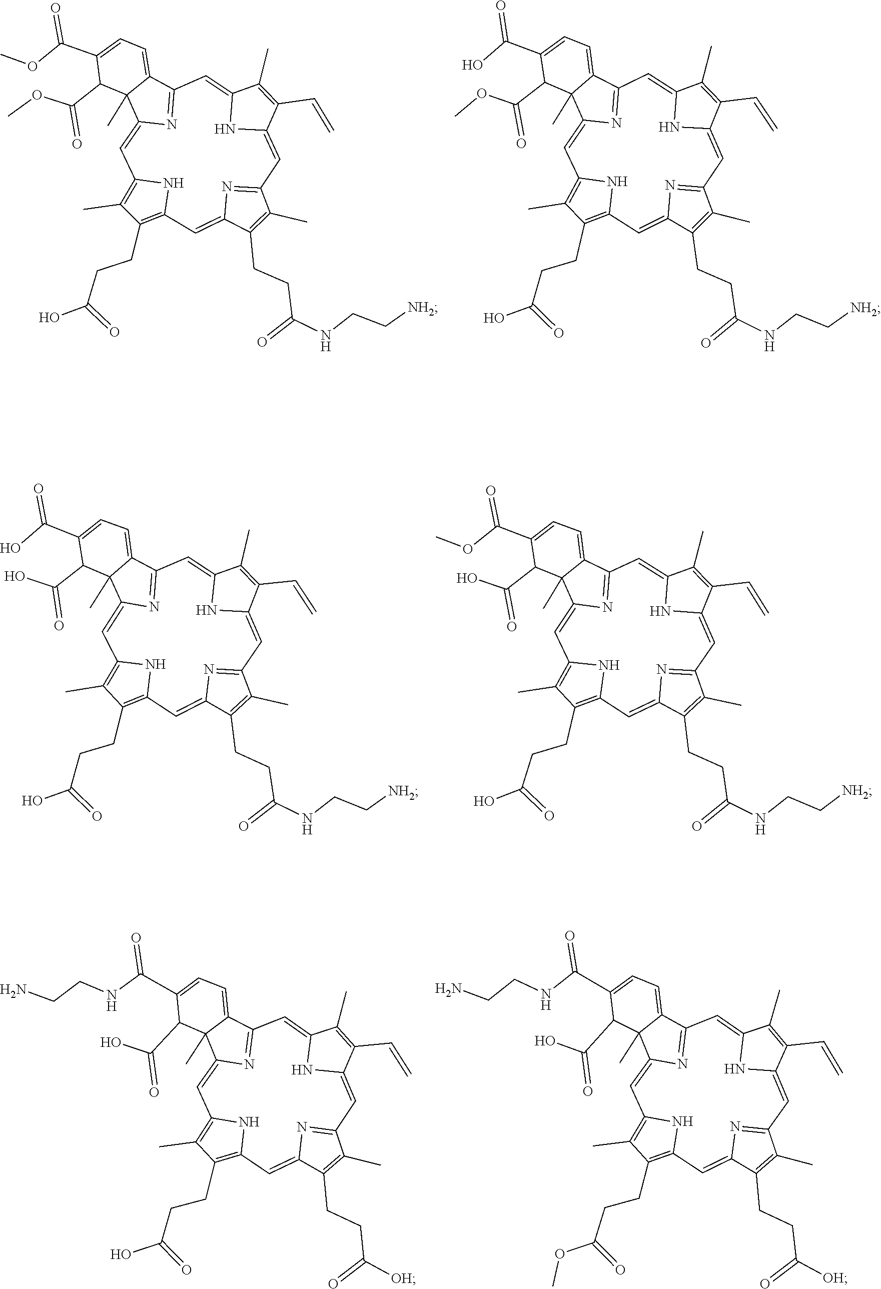 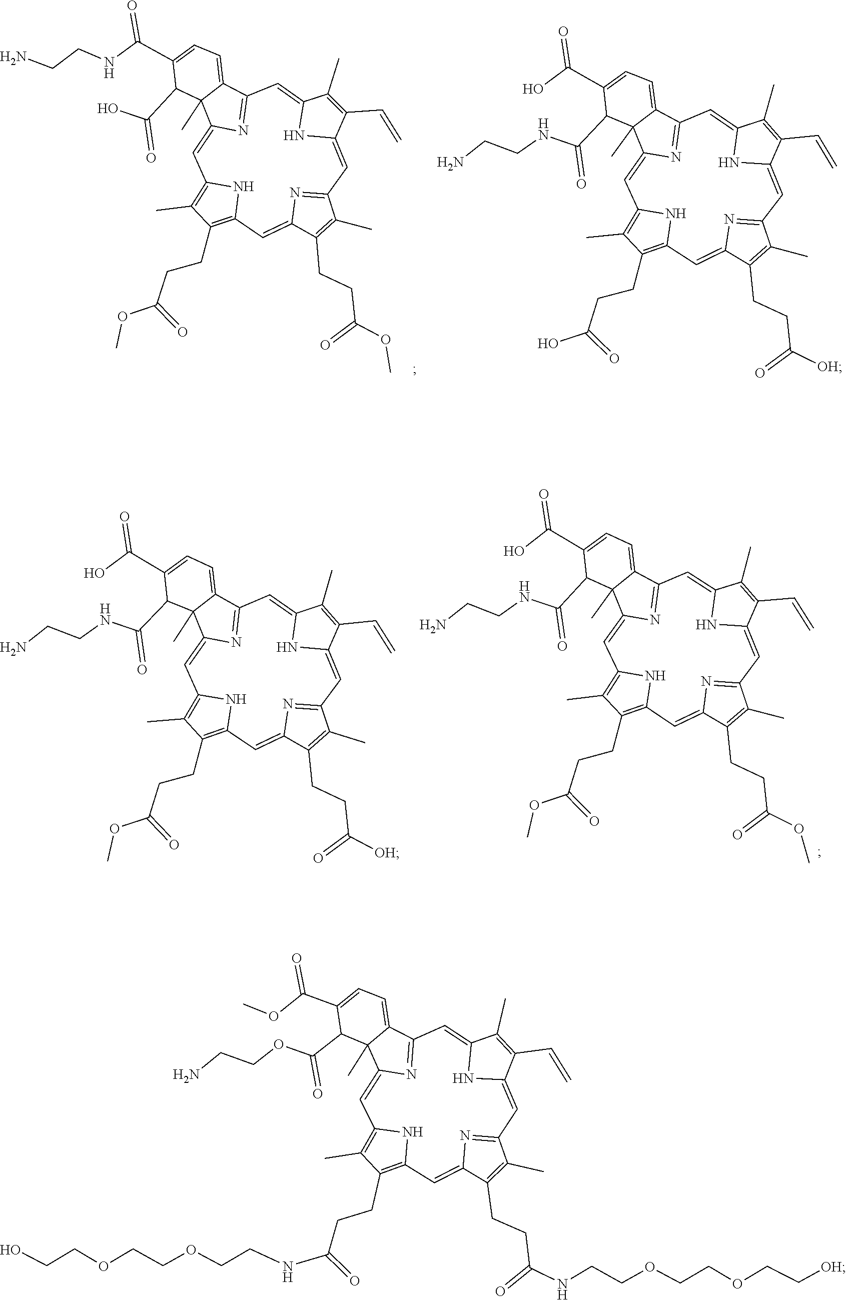 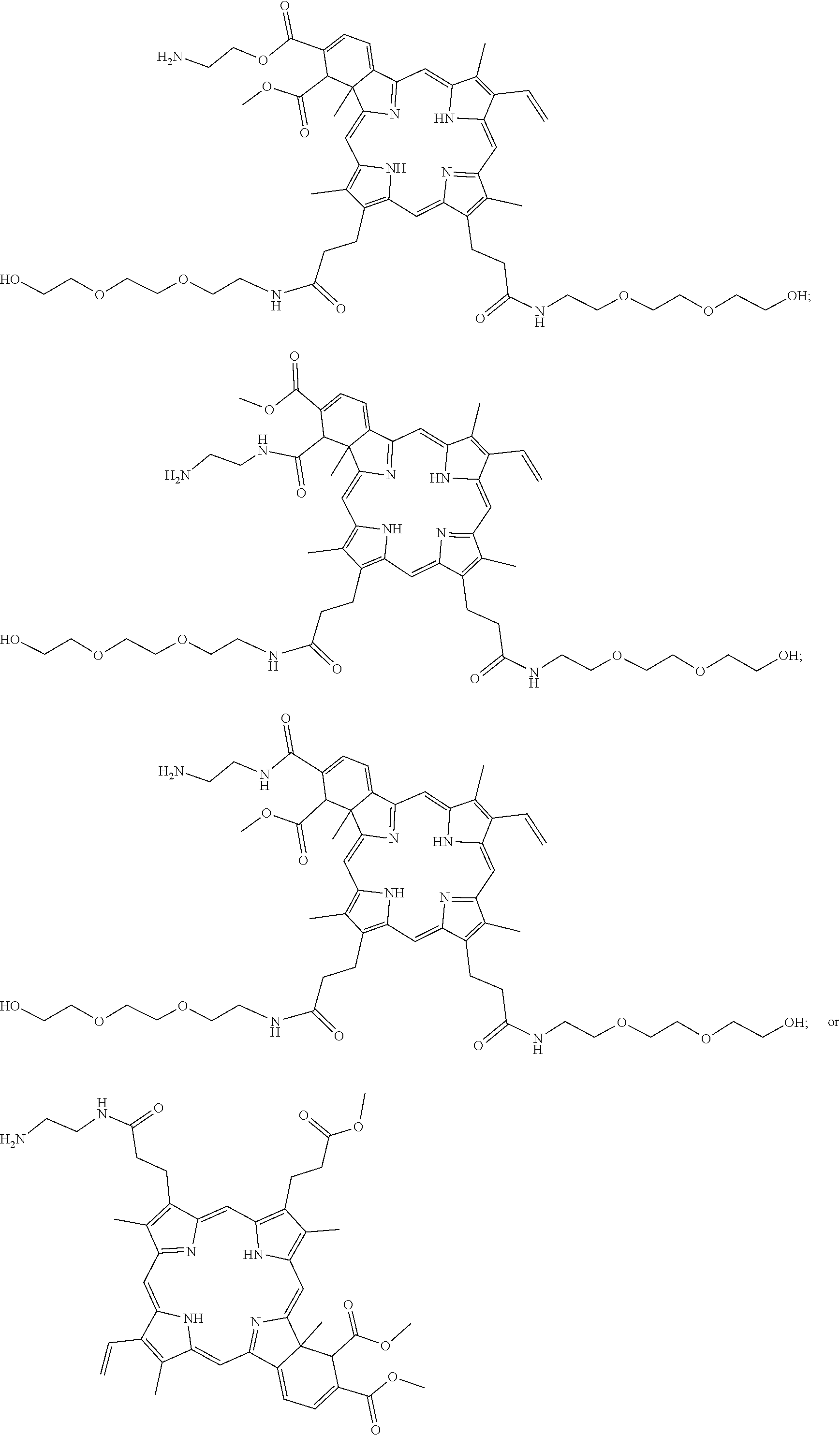 [SMILES: C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)OCCN)C1C(=O)OC ; C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)OC)C1C(=O)NCCN ; C=Cc1c(C)c2cc3nc(cc4[nH]c(cc5nc(cc1[nH]2)C(C)=C5CCC(=O)NCCOCCOCCO)c(CCC(=O)NCCOCCOCCO)c4C)C1(C)C3=CC=C(C(=O)NCCN)C1C(=O)OC ; C=Cc1c(C)c2cc3nc(cc4[nH]c(cc5nc(cc1[nH]2)C(C)=C5CCC(=O)NCCN)c(CCC(=O)OC)c4C)C1(C)C3=CC=C(C(=O)OC)C1C(=O)OC]
or a pharmaceutically acceptable salt thereof, a regioisomer, or a pharmaceutically acceptable salt of the regioisomer.
